(19) United States
(12) Patent Application Publication
(10) Pub. No.: US 2026/0285872 A1
(43) Pub. Date: Sep. 24, 2026

(54) COMPOUNDS AND METHODS FOR DEGRADING GSPT1

(71) Applicant: PIN THERAPEUTICS, INC., Seongnam-si, Gyeonggi-do (KR)

(72) Inventors: Firoz Ali JAIPURI, Fremont, CA (US); James Thomas METZ, Gurnee, IL (US); Mark Rex SPYVEE, Hampstead, NH (US); Heekyu LEE, Seongnam-si (KR); Jinuk KONG, Seongnam-si (KR); Purum KIM, Seongnam-si (KR); Yuna PARK, Seongnam-si (KR); Cheongbi KIM, Seongnam-si (KR)

(73) Assignee: PIN THERAPEUTICS, INC., Seongnam-si, Gyeonggi-do (KR)

(21) Appl. No.: 19/168,395

(22) PCT Filed: Mar. 22, 2024
(86) PCT No.: PCT/IB2024/052779

§ 371 Date: Sep. 24, 2025

Related U.S. Application Data
(60) Provisional application No. 63/454,470, filed on Mar. 24, 2023.

(51) Int. Cl. C07D 487/10; A61K 31/454; A61K 31/4545; A61K 31/4709; A61K 31/4725; A61K 31/496; A61K 31/4995; A61K 31/513; A61K 31/5377; A61K 31/538; A61K 31/5386; A61K 31/55; A61P 35/00; A61P 35/02; C07D 239/22; C07D 401/04; C07D 401/10; C07D 401/14; C07D 403/10; C07D 405/14; C07D 409/14; C07D 413/04; C07D 413/14; C07D 417/14; C07D 471/04; C07D 471/10; C07D 487/08; C07D 491/107; C07D 495/04; C07D 498/08

(52) U.S. Cl. CPC C07D 487/10; A61K 31/454; A61K 31/4545; A61K 31/4709; A61K 31/4725; A61K 31/496; A61K 31/4995; A61K 31/513; A61K 31/5377; A61K 31/538; A61K 31/5386; A61K 31/55; A61P 35/00; A61P 35/02; C07D 239/22; C07D 401/04; C07D 401/10; C07D 401/14; C07D 403/10; C07D 405/14; C07D 409/14; C07D 413/04; C07D 413/14; C07D 417/14; C07D 471/04; C07D 471/10; C07D 487/08; C07D 491/107; C07D 495/04; C07D 498/08

(57) ABSTRACT

Provided are novel compounds which degrade GSPT1 (G1 To S Phase Transition 1), and methods of degrading GSTP1, or preventing or treating GSPT1-related disease by administering the compound to a subject in need thereof.

### BACKGROUND OF THE INVENTION

GSPT1 (G1 To S Phase Transition 1) is an essential gene for cell survival, and its protein product interacts with multiple factors involved in key cellular processes including regulation of cell cycle progression. Accordingly, GSPT1 may impair control of cell proliferation and facilitate cell migration and scar formation. Indeed, GSPT1 has been implicated as an oncogenic driver of several different cancer types, including breast cancer, hepatocellular carcinoma, gastric cancer, and prostate cancer. See, e.g., Brito, et al., Carcinogenesis, Vol. 26, No. 12, pp. 2046-49 (2005); Brito, et al., Canc. Geneti. Cytogen., Vol. 195, pp. 132-42 (2009); Tavassoli, et al., Med. Oncol., Vol. 29, pp. 1581-85 (2011); Wright and Lange, Rev. Urol., Vol. 9, No. 4, pp. 207-213 (2007); Hoshino, et al., Apoptosis, Vol. 17, pp. 1287-99 (2012); Liu, et. al., PLOS One, Vol. 9, No. 1, e86371 (2014); and Jean-Jean, et al., Mol. Cell. Bio., Vol. 27, No. 16, pp. 5619-29 (2007). GSPT1 also contributes to glial scar formation and astrogliosis after a central nervous system (CNS) injury. See, e.g., Ishii et al., J. Biol. Chem., Vol. 292, No. 4, pp. 1240-50 (2017).

In this regard, GSPT1 have been known in the art as targets for preventing or treating various types of cancers. Therefore, there has been a demand for developing GSPT1 inhibitors which may be used for preventing or treating various types of cancers.

Recently, research on targeted protein degradation (TPD) techniques for challenging drug targets has been actively pursued. Proteolytic degradation of cellular proteins aims to treat diseases by removing disease-related proteins using the protein degradation systems present in the body. The irreversibility of proteolysis makes them very suitable as regulatory switches for controlling unidirectional processes.

Accordingly, beyond GSPT1 inhibitor, the development of novel GSPT1 degrader compounds is beneficial for the treatment of GSPT1-related diseases. Especially, since molecular glues utilize small molecule compounds to induce the formation of specific protein complexes facilitating the interaction between the target protein and E3 ligases involved in protein degradation, they offer advantages in developing therapeutics with superior pharmacokinetic properties.

### SUMMARY OF THE INVENTION

The present disclosure provides novel compounds which are represented by Formula (I) as follows, or a pharmaceutically acceptable salt, enantiomer, stereoisomer or prodrug thereof.

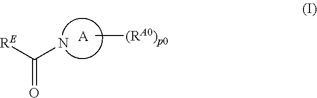

In Formula (I), in some embodiment, R^{E} is selected from a group consisting of

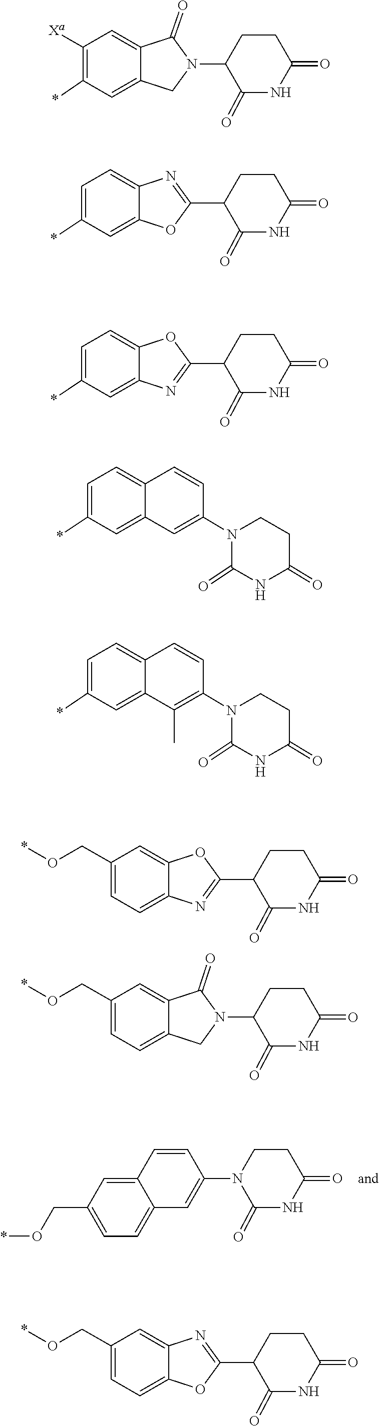

In some embodiment, X^{a} is H or F.

In some embodiment, Ring A is a monocyclic or bicyclic ring (e.g., fused, spiro, bridged) selected from 4-12 membered saturated, partially unsaturated heterocyclic ring, or heteroaryl ring having 0-4 heteroatoms, in addition to the nitrogen already depicted in Ring A, independently selected from nitrogen, oxygen, and sulfur.

In some embodiment, 0-4 methylene moieties (—CH₂—) contained in the Ring A may be independently replaced with oxo (—C═O), —NH—, —N—C₁₋₃ alkyl-, or —O.

In some embodiment, R^{A0} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, N(R^{b})SO₂R^{c}, or a monocyclic or bicyclic ring (e.g., fused, spiro, bridged) optionally substituted with one or more R^{d}.

In some embodiment, the monocyclic or bicyclic ring is selected from a 3-7 membered saturated or partially unsaturated monocyclic carbocyclic ring, a 7-11 membered saturated or partially unsaturated bicyclic carbocyclic ring, a 4-7 membered saturated or partially unsaturated monocyclic heterocyclic ring having 1-4 heteroatoms independently selected from nitrogen, oxygen, and sulfur, a 7-11 membered saturated or partially unsaturated bicyclic heterocyclic ring having 1-5 heteroatoms independently selected from nitrogen, oxygen, and sulfur, a benzene (i.e., phenyl) ring, a 8-10 membered bicyclic aryl ring, a 5-6 membered monocyclic heteroaryl ring having 1-4 heteroatoms independently selected from nitrogen, oxygen, and sulfur, and a 8-10 membered bicyclic heteroaryl ring having 1-5 heteroatoms independently selected from nitrogen, oxygen, and sulfur.

In some embodiment, two of R^{A0} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript p0 is 0, 1, 2, 3, 4, 5, 6, 7, 8, 9, or 10.

In some embodiment, each stereocenter in the compound of Formula above is independently the R-enantiomer, the S-enantiomer or a mixture of R- and S-enantiomers.

In some embodiment, each double bond in the compound of Formula above is independently cis or trans.

In some embodiment, the compound may comprise additional substituent of an optionally substituted ring of Ring A_{1B} as depicted in the Formula (I-a) below:

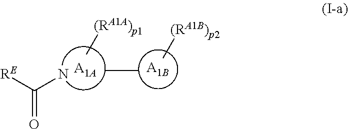

In some embodiment, Ring A_{1A} is a monocyclic or bicyclic ring (e.g., fused, spiro, bridged) selected from 4-12 membered saturated, partially unsaturated heterocyclic ring, or heteroaryl ring having 0-4 heteroatoms, in addition to the nitrogen already depicted in Ring A_{1A}, independently selected from nitrogen, oxygen, and sulfur.

In some embodiment, 0-4 methylene moieties (—CH₂—) contained in the Ring Aia may be independently replaced with oxo (—C═O), —NH—, —N—C₁₋₃ alkyl-, or —O—.

In some embodiment, Ring A_{1B} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur.

In some embodiment, 0-3 methylene moieties (—CH₂—) contained in the Ring A_{1B} may be replaced with oxo (—C═O), —NH—, —N—C₁₋₃ alkyl-, —O—.

In some embodiment, each R^{A1A} and R^{A1B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A1A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{A1B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript p1 and p2 is independently 0, 1, 2, 3, 4, 5, 6, 7, 8, 9, or 10.

In some embodiment, the compound has the structure of Formula (I-a-1):

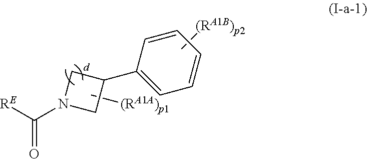

In some embodiment, each R^{A1A} and R^{A1B} is independently hydrogen, deuterium, halogen, C₁-C₆ haloalkyl, or C₁-C₆ haloalkoxy.

In some embodiment, a subscript d is 1 or 2.

In some embodiment, each subscript p1 and p2 is independently 0, 1, 2, or 3.

In some embodiment, R^{E} is

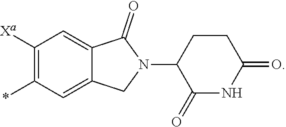

In some embodiment, the compound has the structure of Formula (I-b):

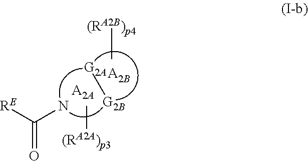

In some embodiment, Ring A_{2A} is a monocyclic heterocyclic ring selected from 3-7 membered saturated, partially unsaturated heterocyclic ring, or 5-6 membered heteroaryl ring having 0-2 heteroatoms, in addition to the nitrogen already depicted in Ring A_{2A}, independently selected from nitrogen, oxygen, and sulfur.

In some embodiment, 0-2 methylene moieties (—CH₂—) contained in the Ring A_{2A} may be independently replaced with oxo (—C═O), —NH—, —N—C₁₋₃ alkyl-, or —O—.

In some embodiment, Ring A_{2B} is a monocyclic ring selected from 3-7 membered saturated or partially unsaturated carbocyclic ring, a 4-7 membered saturated or partially unsaturated monocyclic heterocyclic ring having 1-4 heteroatoms independently selected from nitrogen, oxygen and sulfur, a benzene (i.e., phenyl) ring, or 5-6 membered heteroaryl ring having 1-4 heteroatoms independently selected from nitrogen, oxygen and sulfur.

In some embodiment, 0-3 methylene moieties (—CH₂—) contained in the Ring A_{2B} may be replaced with oxo (—C═O), —NH—, —N—C₁₋₃ alkyl-, or —O—.

In some embodiment, Ring A_{2A} and Ring A_{2B} taken together form a fused-heterocyclic with two shared/border atoms between Ring A_{2A} and Ring A_{2B}, including G_{2A} and G_{2B}.

In some embodiment, each shared/border atoms can be carbon or heteroatom independently selected from nitrogen, oxygen, and sulfur.

In some embodiment, each R^{A2A} and R^{A2B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A2A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{A2B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript p3 and p4 is independently 0, 1, 2, 3, 4, 5, 6, 7, 8, 9, or 10.

In some embodiment, the compound has the structure of selected from Formula (I-b-1) to Formula (I-b-11):

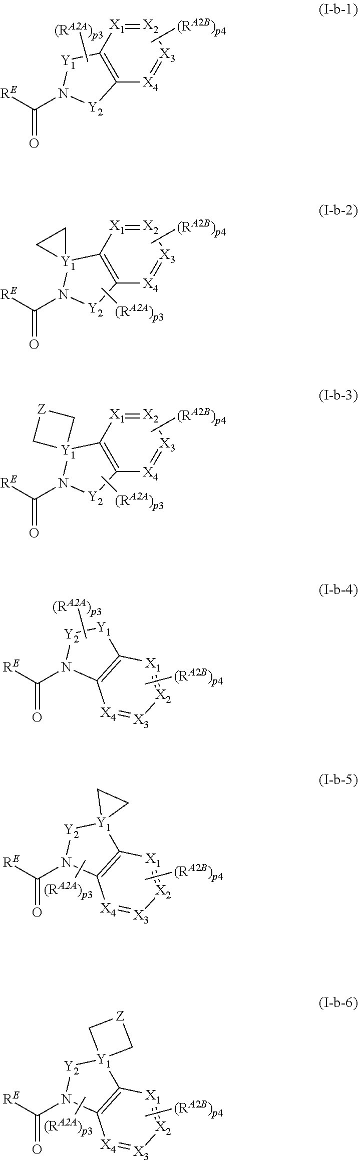

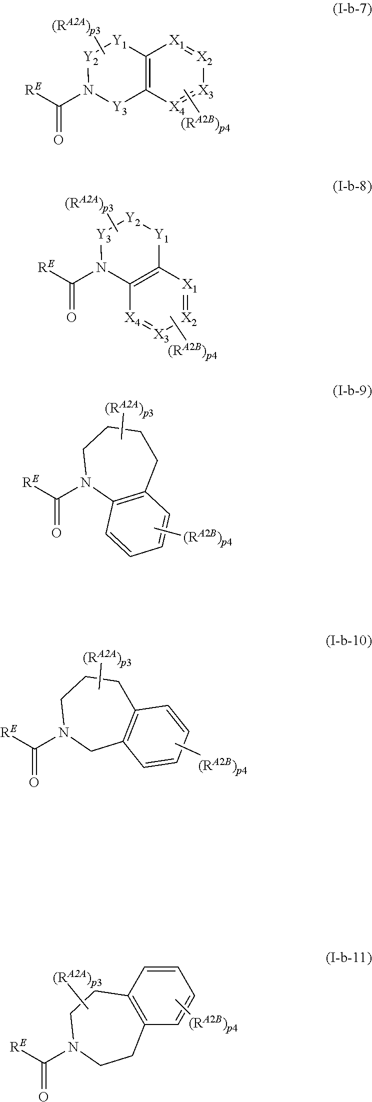

In some embodiment, each of X₁, X₂, X₃ and X₄ is independently N or CH.

In some embodiment, each of Y₁, Y₂ and Y₃ is independently O, NH, N—C₁₋₃ alkyl or CH₂.

In some embodiment, each of Z is independently O, NH, N—C₁₋₃ alkyl or CH₂.

In some embodiment, each R^{A2A} and R^{A2B} is independently hydrogen, deuterium, halogen, C₁-C₄ alkyl (i.e., straight or branched), C₁-C₃ alkoxy, C₁-C₃ haloalkyl, C₁-C₃ haloalkoxy, cyano, or —(CH₂)₁₋₂—NH—C(O)—(C₁₋₃ alkyl).

In some embodiment, the compound has the structure of Formula (I-c):

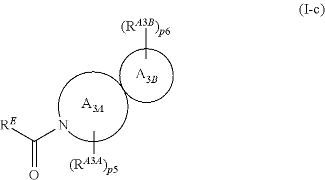

In some embodiment, each Ring A_{3A} and Ring A_{3B} is monocyclic ring selected from 4-7 membered saturated or partially unsaturated ring having 0-4 heteroatoms, in addition to the nitrogen already depicted in Ring A_{3A}, independently selected from nitrogen, oxygen, and sulfur.

In some embodiment, 0-4 methylene moieties (—CH₂—) contained in the Ring A_{3A} or Ring A_{3B} may be independently replaced with oxo (—C═O), —NH—, —N—C₁₋₃ alkyl-, or —O—.

In some embodiment, Ring A_{3A} and Ring A_{3B} taken together form a spiro-heterocyclic with one shared/border carbon atom between Ring A_{3A} and Ring A_{3B}.

In some embodiment, each R^{A3A} and R^{A3B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A3A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{A3B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript p5 and p6 is independently 0, 1, 2, 3, 4, 5, 6, 7, 8, 9, or 10.

In some embodiment, the compound may comprise additional substituent of an optionally substituted ring of Ring A_{3C} as depicted in the Formula (I-c-1) below:

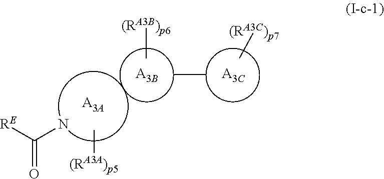

In some embodiment, Ring A_{3C} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur.

In some embodiment, 0-3 methylene moieties (—CH₂—) contained in the Ring A_{3C} may be independently replaced with oxo (—C═O), —NH—, —N—C₁₋₃ alkyl-, or —O—.

In some embodiment, R^{A3C} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A3C} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₅-C₁₃ spiroalkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript p7 is 0, 1, 2, 3, 4, 5, 6, 7, 8, 9, or 10.

In some embodiment, the compound has the structure of Formula (I-c-2) or (I-c-3):

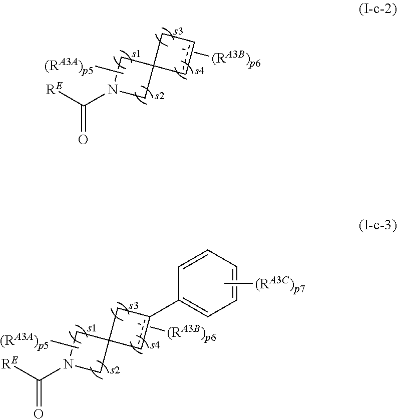

In some embodiment, each subscript s1, s2, s3 and s4 is independently 1 or 2.

In some embodiment, a spiro-heterocyclic ring containing the nitrogen already depicted in Formula (I-c-2) or (I-c-3) above, optionally comprises one double bond therein depicted as dotted line, and 0-2 methylene moieties (—CH₂—) contained in the ring may be replaced with oxo (—C═O), —NH—, —N—C₁₋₃ alkyl-, —O—.

In some embodiment, each R^{A3A}, R^{A3B} and R^{A3C} is independently hydrogen, deuterium, halogen, C₁-C₄ alkyl (i.e., straight or branched), C₁-C₃ alkoxy, C₁-C₃ haloalkyl, C₁-C₃ haloalkoxy, or cyano.

In some embodiment, the compound has the structure selected from Formula (I-c-4) to Formula (I-b-14):

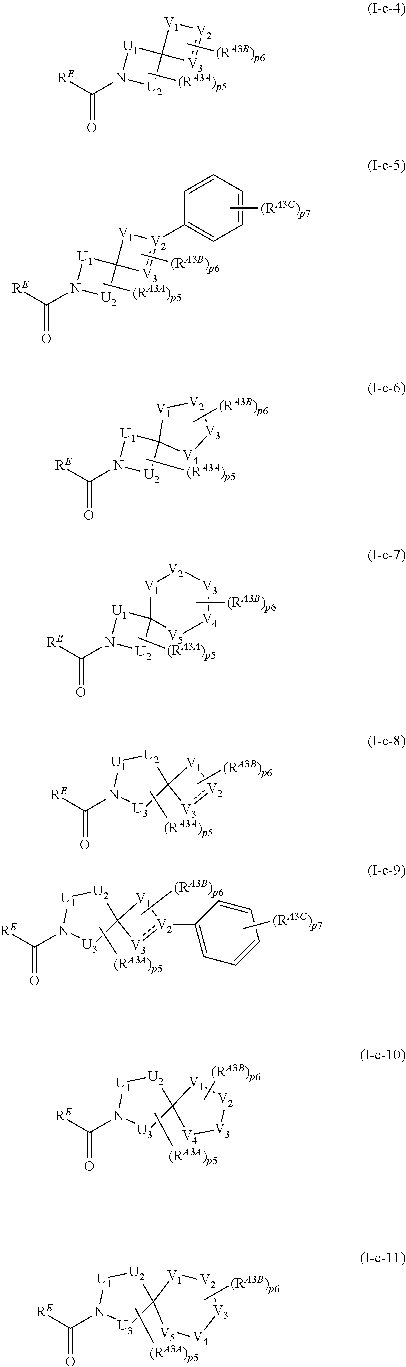

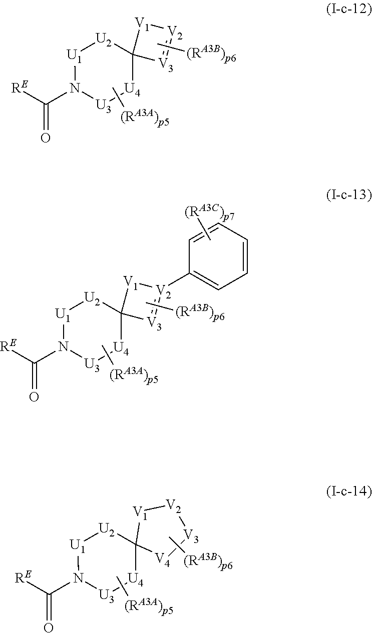

In some embodiment, each of U₁, U₂, U₃ and U₄ is independently C═O, O, NH, N—C₁₋₃ alkyl or CH₂.

In some embodiment, each of V₁, V₂, V₃, V₄ and V₅ is independently C═O, O, NH, N—C₁₋₃ alkyl or CH₂.

In some embodiment, a spiro-heterocyclic ring containing the nitrogen already depicted in Formula (I-c-4) to (I-c-14) above, optionally comprises one double bond therein depicted as dotted line.

In some embodiment, the compound has the structure of Formula (I-d):

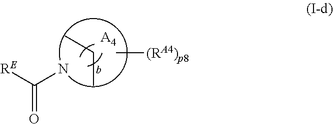

In some embodiment, Ring A₄ is fully saturated 5-10 membered bridged-heterocyclic ring having 0-3 heteroatoms, in addition to the nitrogen already depicted in Ring A₄, independently selected from nitrogen, oxygen, and sulfur.

In some embodiment, 0-3 methylene moieties (—CH₂—) contained in the Ring A₄ may be independently replaced with oxo (—C═O), —NH—, —N—C₁₋₃ alkyl-, or —O—.

In some embodiment, a subscript b is 1, 2 or 3.

In some embodiment, R^{A4} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A4} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript p8 is 0, 1, 2, 3, 4, 5, 6, 7, 8, 9, or 10.

In some embodiment, the compound has the structure of Formula (I-d-1) or (I-d 2):

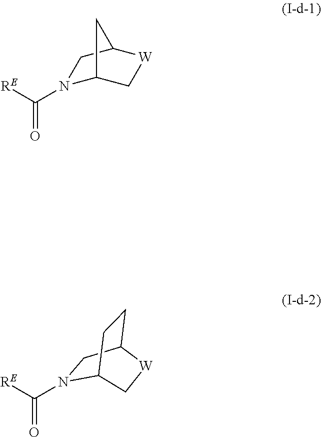

In some embodiment, each W is independently O, NH, N—C₁₋₃ alkyl or CH₂.

The present disclosure provides novel compounds which are represented by Formula (II) as follows, or a pharmaceutically acceptable salt, enantiomer, stereoisomer or prodrug thereof.

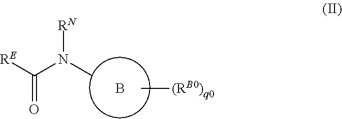

In some embodiment, R^{E} is selected from groups consisting of

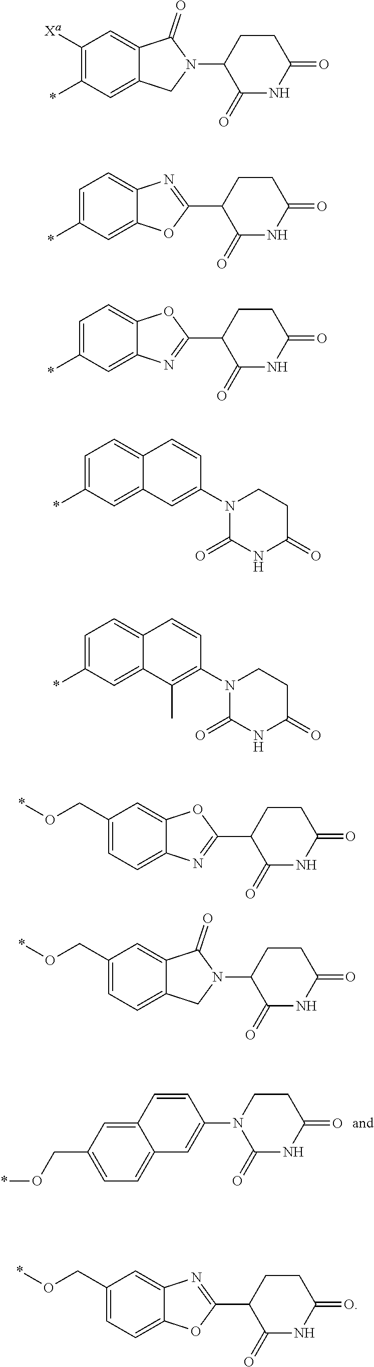

In some embodiment, X^{a} is H or F.

In some embodiment, R^{N} is hydrogen or C₁-C₃ alkyl.

In some embodiment, Ring B is a monocyclic or bicyclic ring (e.g., fused, spiro, bridged) selected from 4-12 membered saturated, partially unsaturated ring having 0-4 heteroatoms independently selected from nitrogen, oxygen, and sulfur.

In some embodiment, 0-4 methylene moieties (—CH₂—) contained in the Ring B may be replaced with oxo (—C═O), —NH—, —N—C₁₋₃ alkyl-, —O—.

In some embodiment, R^{B0} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, N(R^{b})SO₂R^{c}, or a monocyclic or bicyclic ring (e.g., fused, spiro, bridged) optionally substituted with one or more R^{d}.

In some embodiment, the monocyclic or bicyclic ring is selected from a 3-7 membered saturated or partially unsaturated monocyclic carbocyclic ring, a 7-11 membered saturated or partially unsaturated bicyclic carbocyclic ring, a 4-7 membered saturated or partially unsaturated monocyclic heterocyclic ring having 1-4 heteroatoms independently selected from nitrogen, oxygen, and sulfur, a 7-11 membered saturated or partially unsaturated bicyclic heterocyclic ring having 1-5 heteroatoms independently selected from nitrogen, oxygen, and sulfur, a benzene (i.e., phenyl) ring, a 8-10 membered bicyclic aryl ring, a 5-6 membered monocyclic heteroaryl ring having 1-4 heteroatoms independently selected from nitrogen, oxygen, and sulfur, and a 8-10 membered bicyclic heteroaryl ring having 1-5 heteroatoms independently selected from nitrogen, oxygen, and sulfur.

In some embodiment, two of R^{B0} groups on the same atom or adjacent atoms, taken together with their intervening atoms to which they are attached, may optionally form a Spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript q0 is 0, 1, 2, 3, 4, 5, 6, 7, 8, 9, or 10.

In some embodiment, each stereocenter in the compound of Formula above is independently the R-enantiomer, the S-enantiomer or a mixture of R- and S-enantiomers.

In some embodiment, each double bond in the compound of Formula above is independently cis or trans.

In some embodiment, the compound has the structure of Formula (II-b):

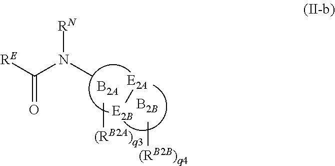

In some embodiment, Ring B_{2A} is a monocyclic heterocyclic ring selected from 3-7 membered saturated, partially unsaturated heterocyclic ring, or 5-6 membered heteroaryl ring having 0-2 heteroatoms independently selected from nitrogen, oxygen, and sulfur.

In some embodiment, 0-2 methylene moieties (—CH₂—) contained in the Ring B_{2A} may be independently replaced with oxo (—C═O), —NH—, —N—C₁₋₃ alkyl-, or —O—.

In some embodiment, Ring B_{2B} is a monocyclic ring selected from 3-7 membered saturated or partially unsaturated carbocyclic ring, a 4-7 membered saturated or partially unsaturated monocyclic heterocyclic ring having 1-4 heteroatoms independently selected from nitrogen, oxygen and sulfur, a benzene (i.e., phenyl) ring, or 5-6 membered heteroaryl ring having 1-4 heteroatoms independently selected from nitrogen, oxygen and sulfur.

In some embodiment, 0-3 methylene moieties (—CH₂—) contained in the Ring B_{2B} may be replaced with oxo (—C═O), —NH—, —N—C₁₋₃ alkyl-, or —O—.

In some embodiment, Ring B_{2A} and Ring B_{2B} taken together form a fused-heterocyclic with two shared/border atoms between Ring B_{2A} and Ring B_{2B}, including E_{2A} and E_{2B}.

In some embodiment, each shared/border atoms can be carbon or heteroatom independently selected from nitrogen, oxygen, and sulfur.

In some embodiment, each R^{B2A} and R^{B2B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B2A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{B2B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R

In some embodiment, each R is independently hydrogen or C₁-C₆ alkyl.

In some embodiment, each subscript q3 and q4 is independently 0, 1, 2, 3, 4, 5, 6, 7, 8, 9, or 10.

In some embodiment, the compound has the structure of Formula (II-c-1):

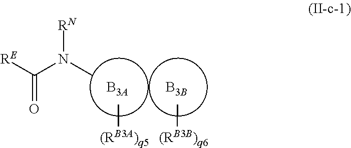

In some embodiment, each Ring B_{3A} and Ring B_{3B} is monocyclic ring selected from 4-7 membered saturated or partially unsaturated ring having 0-4 heteroatoms independently selected from nitrogen, oxygen, and sulfur.

In some embodiment, 0-4 methylene moieties (—CH₂—) contained in the Ring B_{3A} or Ring B_{3B} may be independently replaced with oxo (—C═O), —NH—, —N—C₁₋₃ alkyl-, or —O—.

In some embodiment, Ring B_{3A} and Ring B_{3B} taken together form a spirocyclic or spiro-heterocyclic ring with one shared/border carbon atom between Ring B_{3A} and Ring B_{3B}.

In some embodiment, each R^{B3A} and R^{B3B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R′, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B3A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{B3B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript q5 and q6 is independently 0, 1, 2, 3, 4, 5, 6, 7, 8, 9, or 10.

In some embodiment, the compound comprises additional substituent of an optionally substituted ring of Ring B_{3C} as depicted in the Formula (II-c-2) below:

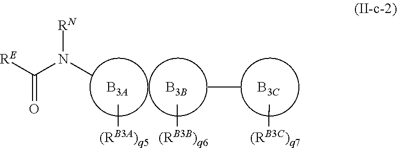

In some embodiment, Ring B_{3C} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur.

In some embodiment, 0-3 methylene moieties (—CH₂—) contained in the Ring B_{3C} may be independently replaced with oxo (—C═O), —NH—, —N—C₁₋₃ alkyl-, or —O—.

In some embodiment, R^{B3C} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B3C} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₅-C₁₃ spiroalkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript q7 is 0, 1, 2, 3, 4, 5, 6, 7, 8, 9, or 10.

In some embodiment, the compound has the structure of Formula (II-c-3) or (I-c-4):

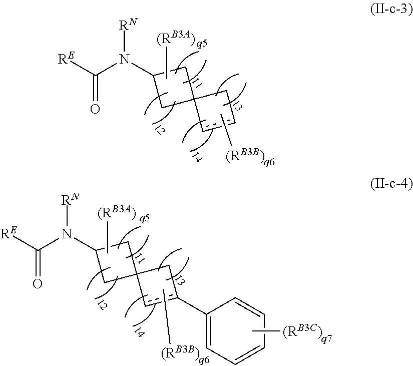

In some embodiment, each subscript t1, t2, t3 and t4 is independently 1 or 2.

In some embodiment, spirocyclic or spiro-heterocyclic ring depicted in Formula (II-c-3) or (II-c-4) above, optionally comprises one double bond therein depicted as dotted line, and 0-2 methylene moieties (—CH₂—) contained in the ring may be replaced with oxo (—C═O), —NH—, —N—C₁₋₃ alkyl-, —O—.

In some embodiment, each R^{B3A}, R^{B3B} and R^{B3C} is independently hydrogen, deuterium, halogen, C₁-C₄ alkyl (i.e., straight or branched), C₁-C₃ alkoxy, C₁-C₃ haloalkyl, C₁-C₃ haloalkoxy, or cyano.

In some embodiment, the compound has the structure of Formula (II-d):

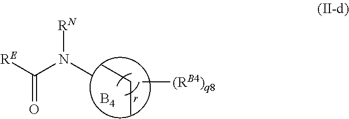

In some embodiment, Ring B₄ is fully saturated 5-10 membered bridged-cyclic ring or bridged-heterocyclic ring having 0-3 heteroatoms independently selected from nitrogen, oxygen, and sulfur.

In some embodiment, 0-3 methylene moieties (—CH₂—) contained in the Ring B₄ may be independently replaced with oxo (—C═O), —NH—, —N—C₁₋₃ alkyl-, or —O—.

In some embodiment, a subscript r is 1, 2 or 3.

In some embodiment, R^{B4} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, N^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B4} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript q8 is 0, 1, 2, 3, 4, 5, 6, 7, 8, 9, or 10.

In some embodiment, the compound has the structure of Formula (II-d-1) or (II-d-2):

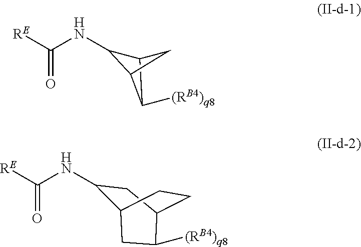

In some embodiment, each R^{B4} is independently hydrogen, deuterium, halogen.

Such a compound above or a pharmaceutically acceptable salt, enantiomer, stereoisomer, or prodrug thereof is useful for degrading GSPT1 (G1 To S Phase Transition 1) protein or reducing protein levels thereof.

In some embodiment, the compound of the present disclosure is for use in degrading GSPT1 protein and/or reducing level thereof.

In some embodiment, the compound of the present disclosure is for use in inhibiting GSPT1 activity.

In some embodiment, the present disclosure provides a pharmaceutical composition comprising: the compound of the present disclosure (represented by Formula (I) or (II)), or the pharmaceutically acceptable salt, enantiomer, stereoisomer or prodrug thereof, and optionally a pharmaceutically acceptable excipient or carrier.

In some embodiment, the pharmaceutical composition is for use in degrading GSPT1 protein and/or reducing level thereof.

In some embodiment, the pharmaceutical composition is for use in inhibiting GSPT1 activity.

The present disclosure also provides a method of degrading GSPT1 protein and/or reducing level thereof in a subject in need thereof, the method comprising administering to the subject an effective amount of the pharmaceutical composition of the present disclosure.

The present disclosure also provides a method of inhibiting GSPT1 activity in a subject in need thereof, the method comprising administering to the subject an effective amount of the pharmaceutical composition of the present disclosure.

The present disclosure also provides a method of treating GSPT1-related disease in a subject in need thereof, the method comprising administering to the subject an effective amount of the compound of the present disclosure (represented by Formula (I) or (II)), or the pharmaceutically acceptable salt, enantiomer, stereoisomer or prodrug thereof, or the pharmaceutical composition of the present disclosure.

In some embodiment, the GSPT1-related disease is cancer, proliferative disorder, inflammation, autoimmune disease, viral infections, or immunological disease.

In some embodiment, the GSPT1-related disease is cancer.

In some embodiment, the cancer is selected from the group consisting of gastric adenocarcinoma, colorectal cancer, li-fraumeni syndrome, esophageal cancer, hepatocellular carcinoma, adenocarcinoma, small intestine cancer, pancreatic cancer, lung cancer, brain cancer, ovarian cancer, cervical cancer, prostate cancer, colon cancer, head and neck cancer, stomach cancer, hepatobiliary cancer, rectal cancer, kidney cancer, bladder cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, liver cancer, skin cancer, urothelial carcinoma, renal cancer, renal cell carcinoma, oropharyngeal cancer, carcinoma, melanoma and breast cancer.

In some embodiment, the cancer is selected from the group consisting of breast cancer, colorectal cancer, lung cancer, e.g. SCLC, NSCLC, liver cancer, neuroendocrine cancer, e.g., neuroendocrine prostate cancer (for example, NEPC (castration-resistant neuroendocrine prostate cancer)) and lung neuroendocrine tumors (Lu-NETs), stomach cancer, pancreatic cancer, gastric cancer, esophageal cancer, bladder cancer, skin cancer, and head and neck cancer.

### DETAILED DESCRIPTION OF THE INVENTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

The present disclosure describes novel compounds which are represented by Formula (I) as follows, or a pharmaceutically acceptable salt, enantiomer, stereoisomer or prodrug thereof. In addition, the present disclosure describes an analog or a radical form of compounds represented by Formula (I).

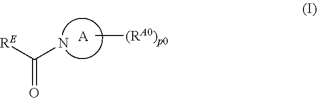

In Formula (I), in some embodiment, R^{E} is selected from a group consisting of

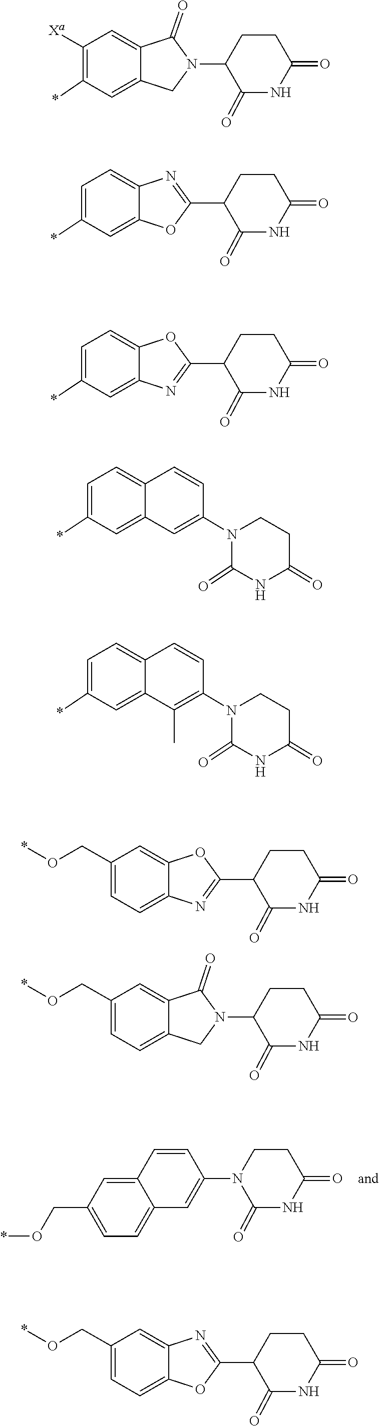

In some embodiment, X^{a} is H or F.

In some embodiment, Ring A is a monocyclic or bicyclic ring (e.g., fused, spiro, bridged) selected from 4-12 membered saturated, partially unsaturated heterocyclic ring, or heteroaryl ring having 0-4 heteroatoms, in addition to the nitrogen already depicted in Ring A, independently selected from nitrogen, oxygen, and sulfur.

In some embodiment, 0-4 methylene moieties (—CH₂—) contained in the Ring A may be independently replaced with oxo (—C═O), —NH—, —N—C₁₋₃ alkyl-, or —O.

In some embodiment, R^{A0} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, N^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, N(R^{b})SO₂R^{c}, or a monocyclic or bicyclic ring (e.g., fused, spiro, bridged) optionally substituted with one or more R^{d}.

In some embodiment, the monocyclic or bicyclic ring is selected from a 3-7 membered saturated or partially unsaturated monocyclic carbocyclic ring, a 7-11 membered saturated or partially unsaturated bicyclic carbocyclic ring, a 4-7 membered saturated or partially unsaturated monocyclic heterocyclic ring having 1-4 heteroatoms independently selected from nitrogen, oxygen, and sulfur, a 7-11 membered saturated or partially unsaturated bicyclic heterocyclic ring having 1-5 heteroatoms independently selected from nitrogen, oxygen, and sulfur, a benzene (i.e., phenyl) ring, a 8-10 membered bicyclic aryl ring, a 5-6 membered monocyclic heteroaryl ring having 1-4 heteroatoms independently selected from nitrogen, oxygen, and sulfur, and a 8-10 membered bicyclic heteroaryl ring having 1-5 heteroatoms independently selected from nitrogen, oxygen, and sulfur.

In some embodiment, two of R^{A0} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript p0 is 0, 1, 2, 3, 4, 5, 6, 7, 8, 9, or 10.

In some embodiment, each stereocenter in the compound of Formula above is independently the R-enantiomer, the S-enantiomer or a mixture of R- and S-enantiomers.

In some embodiment, each double bond in the compound of Formula above is independently cis or trans.

In some embodiment, the compound of the present disclosure is selected from the following [Table A-1].

TABLE A-1

| Example Number | Structure |
|---|---|
| 10 | 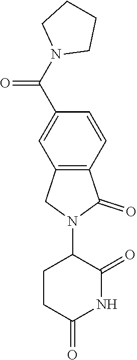 [SMILES: O=C1CCC(N2Cc3cc(C(=O)N4CCCC4)ccc3C2=O)C(=O)N1] |
| 13 | 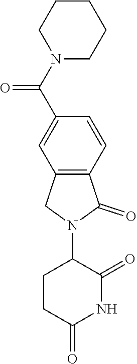 [SMILES: O=C1CCC(N2Cc3cc(C(=O)N4CCCCC4)ccc3C2=O)C(=O)N1] |
| 14 | 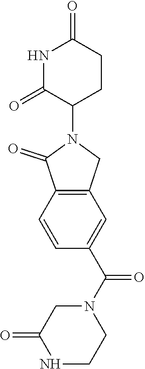 [SMILES: O=C1CN(C(=O)c2ccc3c(c2)CN(C2CCC(=O)NC2=O)C3=O)CCN1] |
| 15 | 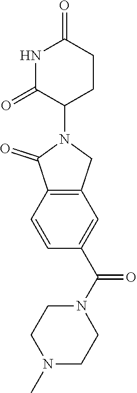 [SMILES: CN1CCN(C(=O)c2ccc3c(c2)CN(C2CCC(=O)NC2=O)C3=O)CC1] |
| 16 | 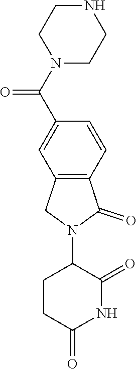 [SMILES: O=C1CCC(N2Cc3cc(C(=O)N4CCNCC4)ccc3C2=O)C(=O)N1] |
| 18 | 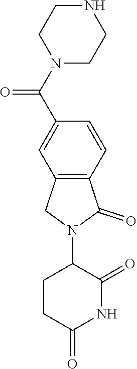 [SMILES: O=C1CCC(N2Cc3cc(C(=O)N4CCNCC4)ccc3C2=O)C(=O)N1] |
| 19 | 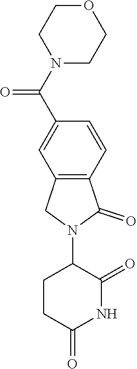 [SMILES: O=C1CCC(N2Cc3cc(C(=O)N4CCOCC4)ccc3C2=O)C(=O)N1] |
| 25 | 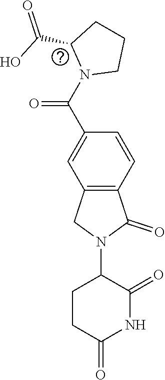 [SMILES: O=C1CCC(N2Cc3cc(C(=O)N4CCC[C@H]4C(=O)O)ccc3C2=O)C(=O)N1] |
| 26 | 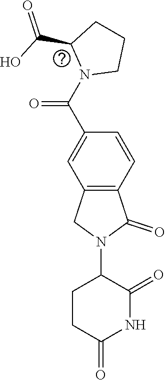 [SMILES: O=C1CCC(N2Cc3cc(C(=O)N4CCC[C@@H]4C(=O)O)ccc3C2=O)C(=O)N1] |
| 27 | 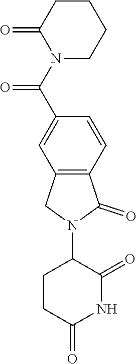 [SMILES: O=C1CCC(N2Cc3cc(C(=O)N4CCCCC4=O)ccc3C2=O)C(=O)N1] |
| 37 | 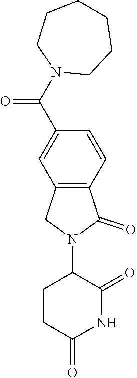 [SMILES: O=C1CCC(N2Cc3cc(C(=O)N4CCCCCC4)ccc3C2=O)C(=O)N1] |

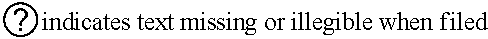

In some embodiment, the compound may comprise additional substituent of an optionally substituted ring of Ring A_{1B} as depicted in the Formula (I-a) below:

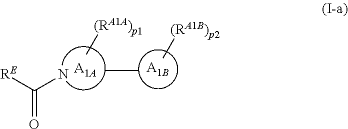

In some embodiment, Ring A_{1A} is a monocyclic or bicyclic ring (e.g., fused, spiro, bridged) selected from 4-12 membered saturated, partially unsaturated heterocyclic ring, or heteroaryl ring having 0-4 heteroatoms, in addition to the nitrogen already depicted in Ring A_{1A}, independently selected from nitrogen, oxygen, and sulfur.

In some embodiment, 0-4 methylene moieties (—CH₂—) contained in the Ring A_{1A} may be independently replaced with oxo (—C═O), —NH—, —N—C₁₋₃ alkyl-, or —O—.

In some embodiment, Ring A_{1B} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur.

In some embodiment, 0-3 methylene moieties (—CH₂—) contained in the Ring A_{1B} may be replaced with oxo (—C═O), —NH—, —N—C₁₋₃ alkyl-, —O—.

In some embodiment, each R^{A1A} and R^{A1B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A1A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{A1B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript p1 and p2 is independently 0, 1, 2, 3, 4, 5, 6, 7, 8, 9, or 10.

In some embodiment, the compound has the structure of Formula (I-a-1):

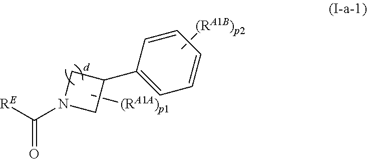

In some embodiment, each R^{A1A} and R^{A1B} is independently hydrogen, deuterium, halogen, C₁-C₆ haloalkyl, or C₁-C₆ haloalkoxy.

In some embodiment, a subscript d is 1 or 2.

In some embodiment, each subscript p1 and p2 is independently 0, 1, 2, or 3.

In some embodiment, R^{E} is

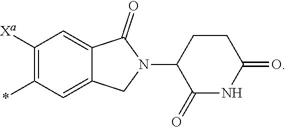

In some embodiment, the compound of the present disclosure is selected from the following [Table A-2].

TABLE A-2

| Example Number | Structure |
|---|---|
| 48 | 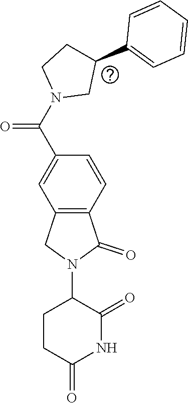 [SMILES: O=C1CCC(N2Cc3cc(C(=O)N4CC[C@@H](c5ccccc5)C4)ccc3C2=O)C(=O)N1] |
| 49 | 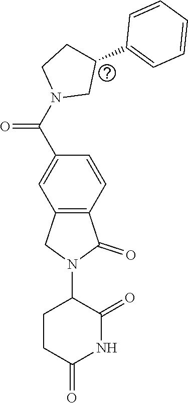 [SMILES: O=C1CCC(N2Cc3cc(C(=O)N4CC[C@H](c5ccccc5)C4)ccc3C2=O)C(=O)N1] |
| 50 | 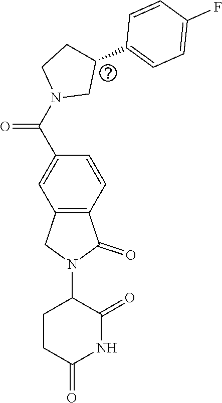 [SMILES: O=C1CCC(N2Cc3cc(C(=O)N4CC[C@H](c5ccc(F)cc5)C4)ccc3C2=O)C(=O)N1] |
| 51 | 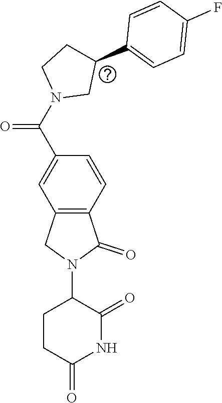 [SMILES: O=C1CCC(N2Cc3cc(C(=O)N4CC[C@@H](c5ccc(F)cc5)C4)ccc3C2=O)C(=O)N1] |
| 52 | 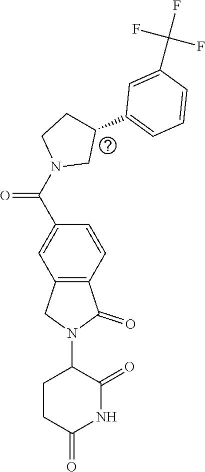 [SMILES: O=C1CCC(N2Cc3cc(C(=O)N4CC[C@H](c5cccc(C(F)(F)F)c5)C4)ccc3C2=O)C(=O)N1] |
| 53 | 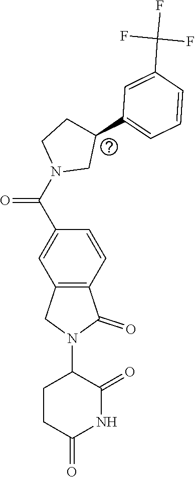 [SMILES: O=C1CCC(N2Cc3cc(C(=O)N4CC[C@@H](c5cccc(C(F)(F)F)c5)C4)ccc3C2=O)C(=O)N1] |
| 54 | 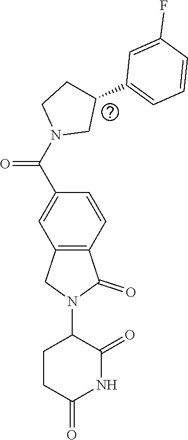 [SMILES: O=C1CCC(N2Cc3cc(C(=O)N4CC[C@H](c5cccc(F)c5)C4)ccc3C2=O)C(=O)N1] |
| 55 | 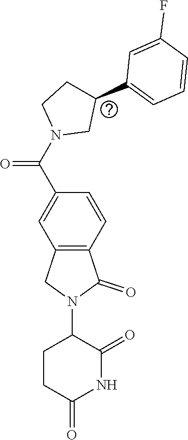 [SMILES: O=C1CCC(N2Cc3cc(C(=O)N4CC[C@@H](c5cccc(F)c5)C4)ccc3C2=O)C(=O)N1] |
| 170 | 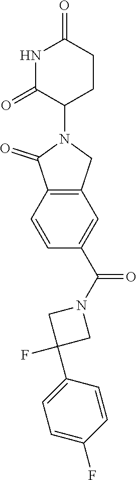 [SMILES: O=C1CCC(N2Cc3cc(C(=O)N4CC(F)(c5ccc(F)cc5)C4)ccc3C2=O)C(=O)N1] |
| 191 | 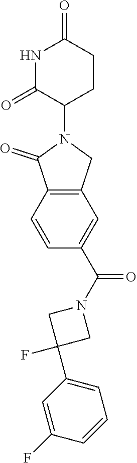 [SMILES: O=C1CCC(N2Cc3cc(C(=O)N4CC(F)(c5cccc(F)c5)C4)ccc3C2=O)C(=O)N1] |
| 192 | 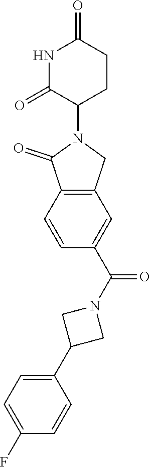 [SMILES: O=C1CCC(N2Cc3cc(C(=O)N4CC(c5ccc(F)cc5)C4)ccc3C2=O)C(=O)N1] |
| 193 | 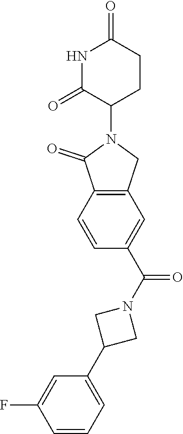 [SMILES: O=C1CCC(N2Cc3cc(C(=O)N4CC(c5cccc(F)c5)C4)ccc3C2=O)C(=O)N1] |
| 194 | 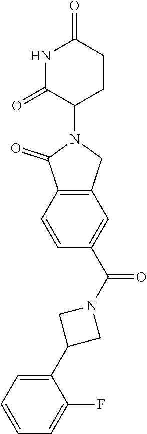 [SMILES: O=C1CCC(N2Cc3cc(C(=O)N4CC(c5ccccc5F)C4)ccc3C2=O)C(=O)N1] |
| 202 | 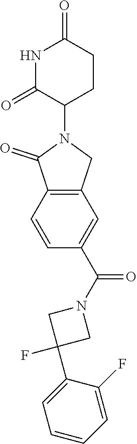 [SMILES: O=C1CCC(N2Cc3cc(C(=O)N4CC(F)(c5ccccc5F)C4)ccc3C2=O)C(=O)N1] |

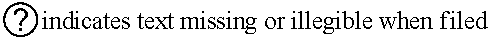

In some embodiment, the compound has the structure of Formula (I-b):

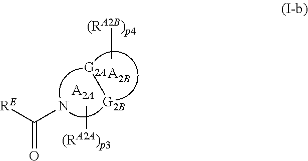

In some embodiment, Ring A_{2A} is a monocyclic heterocyclic ring selected from 3-7 membered saturated, partially unsaturated heterocyclic ring, or 5-6 membered heteroaryl ring having 0-2 heteroatoms, in addition to the nitrogen already depicted in Ring A_{2A}, independently selected from nitrogen, oxygen, and sulfur.

In some embodiment, 0-2 methylene moieties (—CH₂—) contained in the Ring A_{2A} may be independently replaced with oxo (—C═O), —NH—, —N—C₁₋₃ alkyl-, or —O—.

In some embodiment, Ring A_{2B} is a monocyclic ring selected from 3-7 membered saturated or partially unsaturated carbocyclic ring, a 4-7 membered saturated or partially unsaturated monocyclic heterocyclic ring having 1-4 heteroatoms independently selected from nitrogen, oxygen and sulfur, a benzene (i.e., phenyl) ring, or 5-6 membered heteroaryl ring having 1-4 heteroatoms independently selected from nitrogen, oxygen and sulfur.

In some embodiment, 0-3 methylene moieties (—CH₂—) contained in the Ring A_{2B} may be replaced with oxo (—C═O), —NH—, —N—C₁₋₃ alkyl-, or —O—.

In some embodiment, Ring A_{2A} and Ring A_{2B} taken together form a fused-heterocyclic with two shared/border atoms between Ring A_{2A} and Ring A_{2B}, including G_{2A} and G_{2B}.

In some embodiment, each shared/border atoms can be carbon or heteroatom independently selected from nitrogen, oxygen, and sulfur.

In some embodiment, each R^{A2A} and R^{A2B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R′, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A2A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{A2B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript p3 and p4 is independently 0, 1, 2, 3, 4, 5, 6, 7, 8, 9, or 10.

In some embodiment, the compound has the structure of selected from Formula (I-b-1) to Formula (I-b-11):

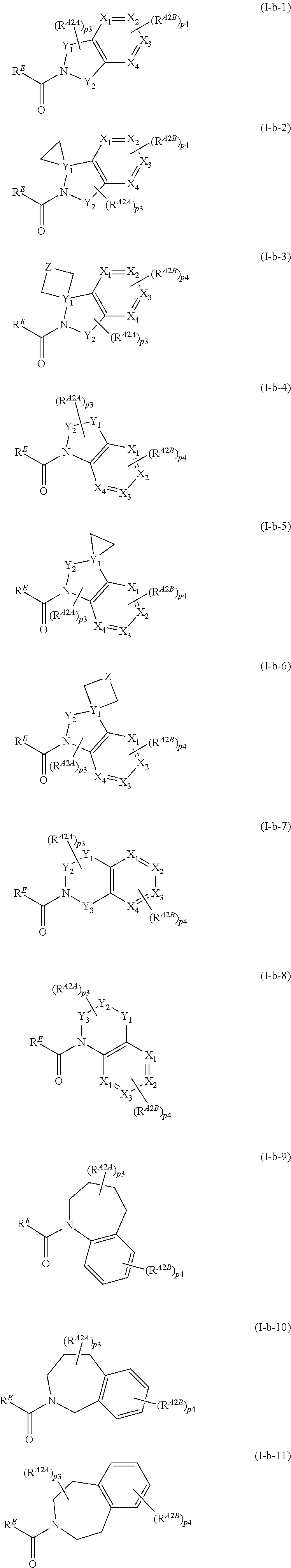

In some embodiment, each of X₁, X₂, X₃ and X₄ is independently N or CH.

In some embodiment, each of Y₁, Y₂ and Y₃ is independently O, NH, N—C₁₋₃ alkyl or CH₂.

In some embodiment, each of Z is independently O, NH, N—C₁₋₃ alkyl or CH₂.

In some embodiment, each R^{A2A} and R^{A2B} is independently hydrogen, deuterium, halogen, C₁-C₄ alkyl (i.e., straight or branched), C₁-C₃ alkoxy, C₁-C₃ haloalkyl, C₁-C₃ haloalkoxy, cyano, or —(CH₂)₁₋₂—NH—C(O)—(C₁₋₃ alkyl).

In some embodiment, the compound of the present disclosure is selected from the following [Table A-3].

TABLE A-3

| Example Number | Structure |
|---|---|
| 11 | 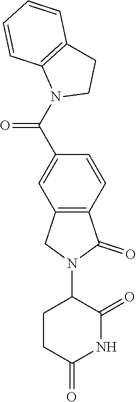 [SMILES: O=C1CCC(N2Cc3cc(C(=O)N4CCc5ccccc54)ccc3C2=O)C(=O)N1] |
| 12 | 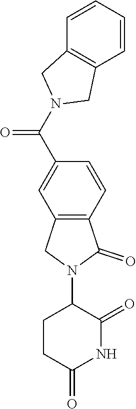 [SMILES: O=C1CCC(N2Cc3cc(C(=O)N4Cc5ccccc5C4)ccc3C2=O)C(=O)N1] |
| 17 | 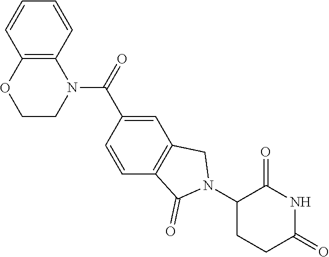 [SMILES: O=C1CCC(N2Cc3cc(C(=O)N4CCOc5ccccc54)ccc3C2=O)C(=O)N1] |
| 20 | 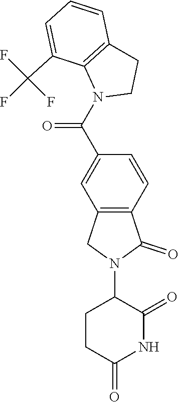 [SMILES: O=C1CCC(N2Cc3cc(C(=O)N4CCc5cccc(C(F)(F)F)c54)ccc3C2=O)C(=O)N1] |
| 21 | 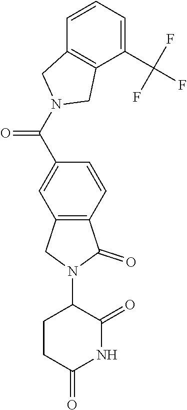 [SMILES: O=C1CCC(N2Cc3cc(C(=O)N4Cc5cccc(C(F)(F)F)c5C4)ccc3C2=O)C(=O)N1] |
| 22 | 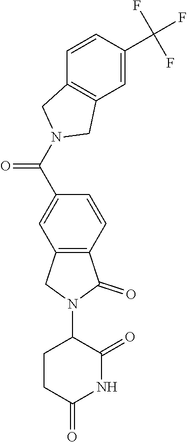 [SMILES: O=C1CCC(N2Cc3cc(C(=O)N4Cc5ccc(C(F)(F)F)cc5C4)ccc3C2=O)C(=O)N1] |
| 23 | 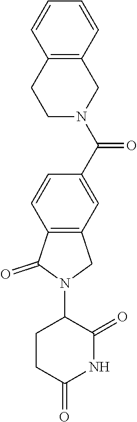 [SMILES: O=C1CCC(N2Cc3cc(C(=O)N4CCc5ccccc5C4)ccc3C2=O)C(=O)N1] |
| 24 | 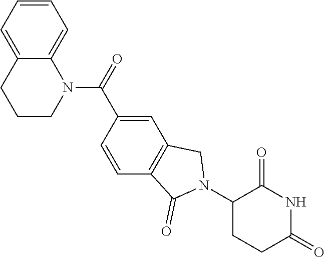 [SMILES: O=C1CCC(N2Cc3cc(C(=O)N4CCCc5ccccc54)ccc3C2=O)C(=O)N1] |
| 28 | 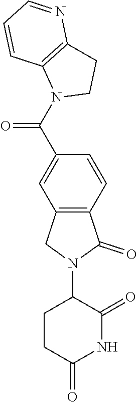 [SMILES: O=C1CCC(N2Cc3cc(C(=O)N4CCc5ncccc54)ccc3C2=O)C(=O)N1] |
| 29 | 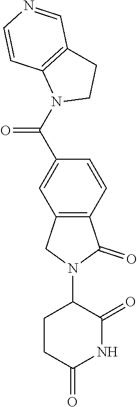 [SMILES: O=C1CCC(N2Cc3cc(C(=O)N4CCc5cnccc54)ccc3C2=O)C(=O)N1] |
| 30 | 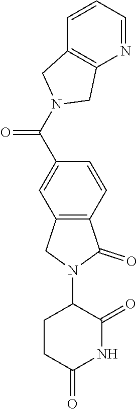 [SMILES: O=C1CCC(N2Cc3cc(C(=O)N4Cc5cccnc5C4)ccc3C2=O)C(=O)N1] |
| 31 | 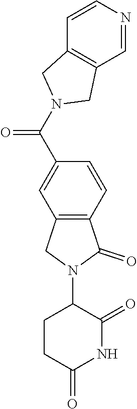 [SMILES: O=C1CCC(N2Cc3cc(C(=O)N4Cc5ccncc5C4)ccc3C2=O)C(=O)N1] |
| 32 | 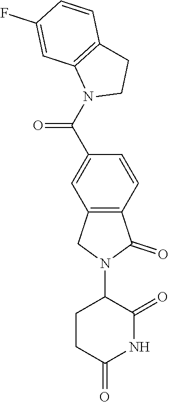 [SMILES: O=C1CCC(N2Cc3cc(C(=O)N4CCc5ccc(F)cc54)ccc3C2=O)C(=O)N1] |
| 33 | 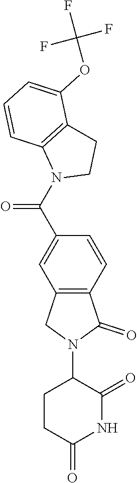 [SMILES: O=C1CCC(N2Cc3cc(C(=O)N4CCc5c(OC(F)(F)F)cccc54)ccc3C2=O)C(=O)N1] |
| 34 | 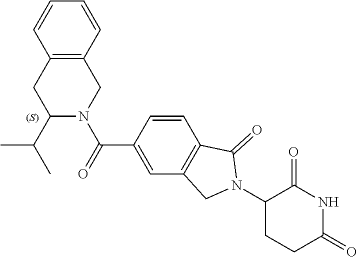 |
| 35 | 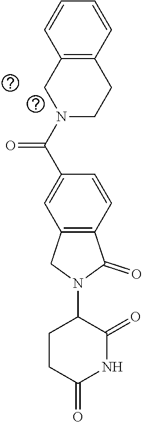 [SMILES: O=C1CCC(N2Cc3cc(C(=O)N4CCc5ccccc5C4)ccc3C2=O)C(=O)N1] |
| 36 | 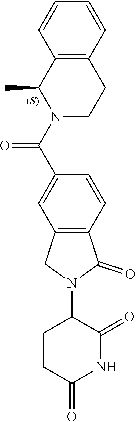 |
| 38 | 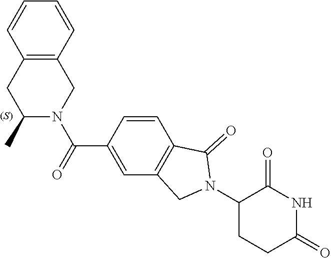 |
| 39 | 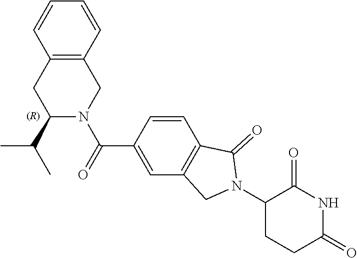 |
| 40 | 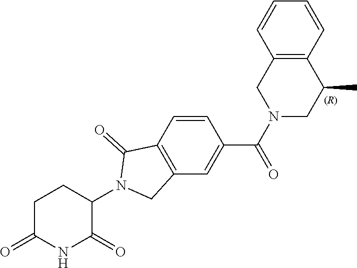 |
| 41 | 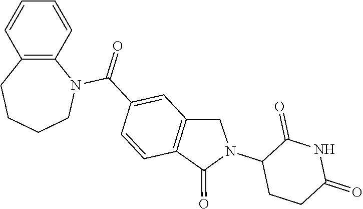 [SMILES: O=C1CCC(N2Cc3cc(C(=O)N4CCCCc5ccccc54)ccc3C2=O)C(=O)N1] |
| 42 | 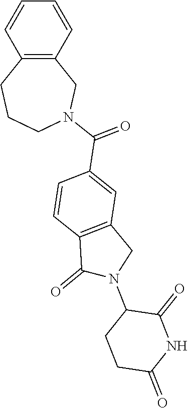 [SMILES: O=C1CCC(N2Cc3cc(C(=O)N4CCCc5ccccc5C4)ccc3C2=O)C(=O)N1] |
| 43 | 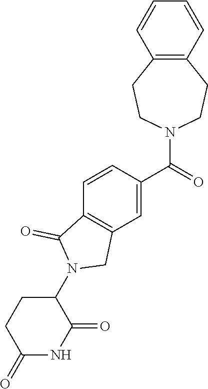 [SMILES: O=C1CCC(N2Cc3cc(C(=O)N4CCc5ccccc5CC4)ccc3C2=O)C(=O)N1] |
| 44 | 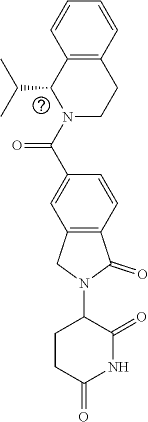 [SMILES: CC(C)[C@@H]1c2ccccc2CCN1C(=O)c1ccc2c(c1)CN(C1CCC(=O)NC1=O)C2=O] |
| 45 | 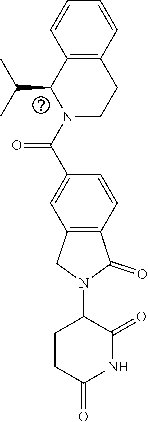 [SMILES: CC(C)[C@H]1c2ccccc2CCN1C(=O)c1ccc2c(c1)CN(C1CCC(=O)NC1=O)C2=O] |
| 46 | 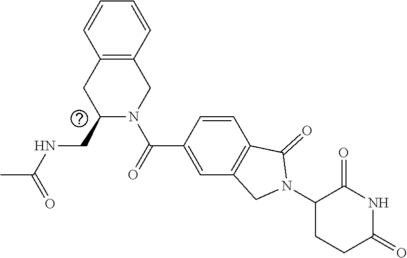 [SMILES: CC(=O)NC[C@H]1Cc2ccccc2CN1C(=O)c1ccc2c(c1)CN(C1CCC(=O)NC1=O)C2=O] |
| 47 | 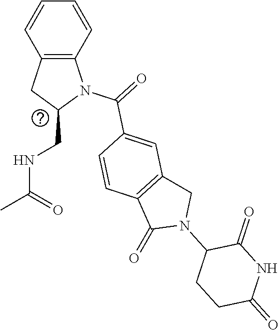 [SMILES: CC(=O)NC[C@H]1Cc2ccccc2N1C(=O)c1ccc2c(c1)CN(C1CCC(=O)NC1=O)C2=O] |
| 56 | 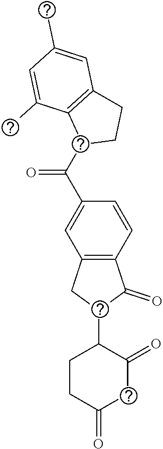 [SMILES: Cc1cc(C)c2c(c1)CCN2C(=O)c1ccc2c(c1)CN(C1CCC(=O)CC1=O)C2=O] |
| 57 | 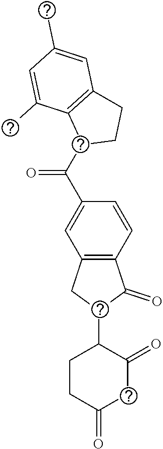 [SMILES: Cc1cc(C)c2c(c1)CCN2C(=O)c1ccc2c(c1)CN(C1CCC(=O)CC1=O)C2=O] |
| 63 | 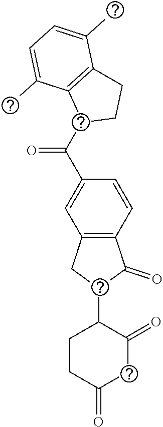 [SMILES: Cc1ccc(C)c2c1CCN2C(=O)c1ccc2c(c1)CN(C1CCC(=O)CC1=O)C2=O] |
| 64 | 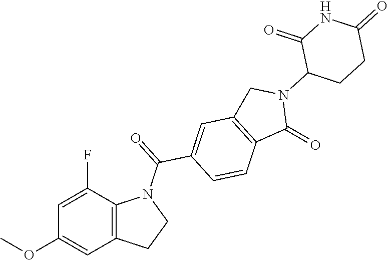 [SMILES: COc1cc(F)c2c(c1)CCN2C(=O)c1ccc2c(c1)CN(C1CCC(=O)NC1=O)C2=O] |
| 65 | 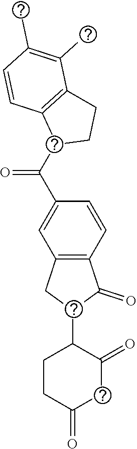 [SMILES: Cc1ccc2c(c1C)CCN2C(=O)c1ccc2c(c1)CN(C1CCC(=O)CC1=O)C2=O] |
| 66 | 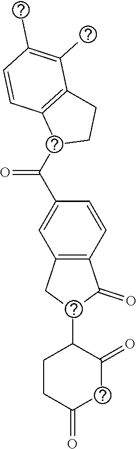 [SMILES: Cc1ccc2c(c1C)CCN2C(=O)c1ccc2c(c1)CN(C1CCC(=O)CC1=O)C2=O] |
| 73 | 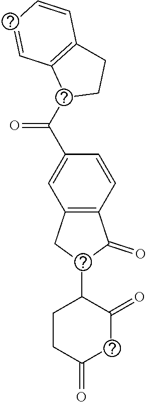 [SMILES: O=C1CCC(N2Cc3cc(C(=O)N4CCc5ccccc54)ccc3C2=O)C(=O)C1] |
| 74 | 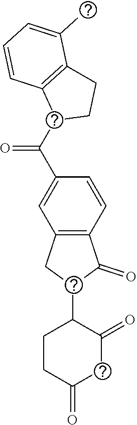 [SMILES: Cc1cccc2c1CCN2C(=O)c1ccc2c(c1)CN(C1CCC(=O)CC1=O)C2=O] |
| 75 | 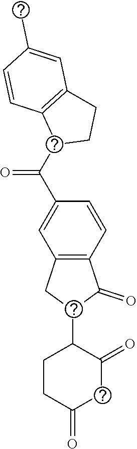 [SMILES: Cc1ccc2c(c1)CCN2C(=O)c1ccc2c(c1)CN(C1CCC(=O)CC1=O)C2=O] |
| 76 | 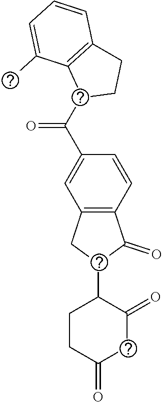 [SMILES: Cc1cccc2c1N(C(=O)c1ccc3c(c1)CN(C1CCC(=O)CC1=O)C3=O)CC2] |
| 77 | 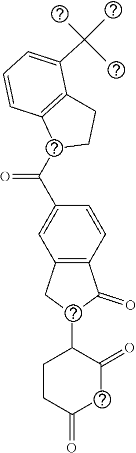 [SMILES: CC(C)(C)c1cccc2c1CCN2C(=O)c1ccc2c(c1)CN(C1CCC(=O)CC1=O)C2=O] |
| 78 | 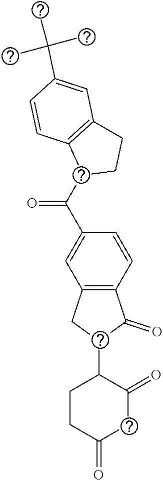 [SMILES: CC(C)(C)c1ccc2c(c1)CCN2C(=O)c1ccc2c(c1)CN(C1CCC(=O)CC1=O)C2=O] |
| 79 | 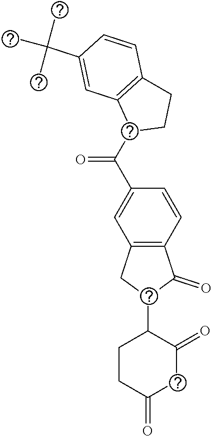 [SMILES: CC(C)(C)c1ccc2c(c1)N(C(=O)c1ccc3c(c1)CN(C1CCC(=O)CC1=O)C3=O)CC2] |
| 80 | 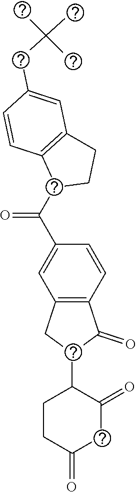 [SMILES: CC(C)(C)Cc1ccc2c(c1)CCN2C(=O)c1ccc2c(c1)CN(C1CCC(=O)CC1=O)C2=O] |
| 81 | 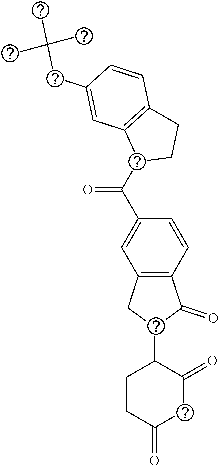 [SMILES: CC(C)(C)Cc1ccc2c(c1)N(C(=O)c1ccc3c(c1)CN(C1CCC(=O)CC1=O)C3=O)CC2] |
| 82 | 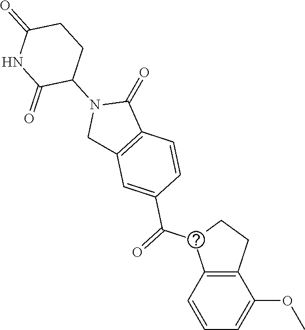 [SMILES: COc1cccc2c1CCC2C(=O)c1ccc2c(c1)CN(C1CCC(=O)NC1=O)C2=O] |
| 83 | 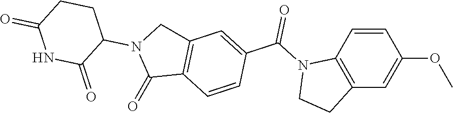 [SMILES: COc1ccc2c(c1)CCN2C(=O)c1ccc2c(c1)CN(C1CCC(=O)NC1=O)C2=O] |
| 84 | 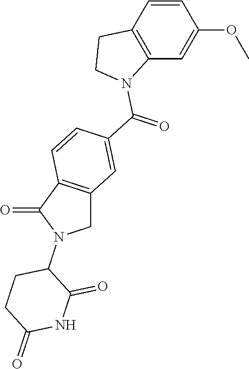 [SMILES: COc1ccc2c(c1)N(C(=O)c1ccc3c(c1)CN(C1CCC(=O)NC1=O)C3=O)CC2] |
| 85 | 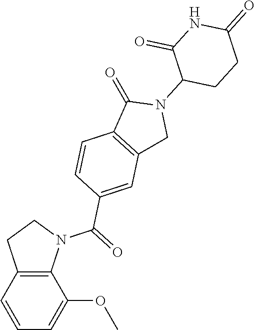 [SMILES: COc1cccc2c1N(C(=O)c1ccc3c(c1)CN(C1CCC(=O)NC1=O)C3=O)CC2] |
| 86 | 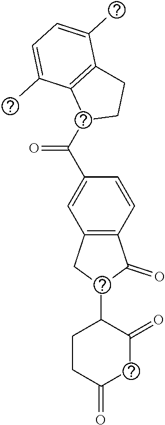 [SMILES: Cc1ccc(C)c2c1CCN2C(=O)c1ccc2c(c1)CN(C1CCC(=O)CC1=O)C2=O] |
| 87 | 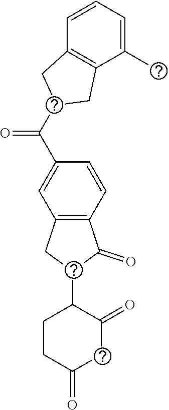 [SMILES: Cc1cccc2c1CN(C(=O)c1ccc3c(c1)CN(C1CCC(=O)CC1=O)C3=O)C2] |
| 88 | 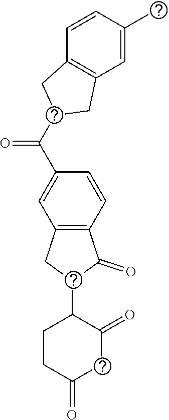 [SMILES: Cc1ccc2c(c1)CN(C(=O)c1ccc3c(c1)CN(C1CCC(=O)CC1=O)C3=O)C2] |
| 92 | 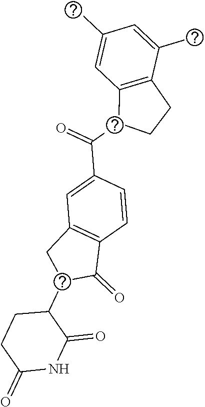 [SMILES: Cc1cc(C)c2c(c1)C(C(=O)c1ccc3c(c1)CC(C1CCC(=O)NC1=O)C3=O)CC2] |
| 101 | 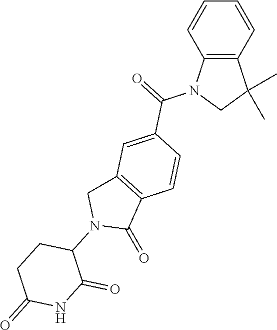 [SMILES: CC1(C)CN(C(=O)c2ccc3c(c2)CN(C2CCC(=O)NC2=O)C3=O)c2ccccc21] |
| 102 | 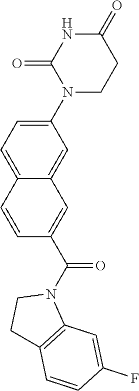 [SMILES: O=C1CCN(c2ccc3ccc(C(=O)N4CCc5ccc(F)cc54)cc3c2)C(=O)N1] |
| 103 | 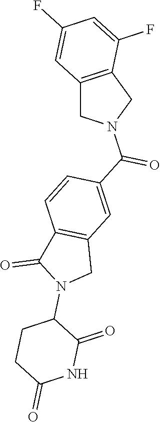 [SMILES: O=C1CCC(N2Cc3cc(C(=O)N4Cc5cc(F)cc(F)c5C4)ccc3C2=O)C(=O)N1] |
| 104 | 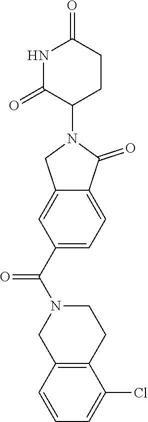 [SMILES: O=C1CCC(N2Cc3cc(C(=O)N4CCc5c(Cl)cccc5C4)ccc3C2=O)C(=O)N1] |
| 105 | 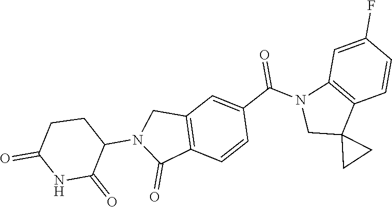 [SMILES: O=C1CCC(N2Cc3cc(C(=O)N4CC5(CC5)c5ccc(F)cc54)ccc3C2=O)C(=O)N1] |
| 106 | 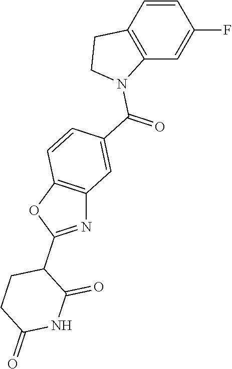 [SMILES: O=C1CCC(c2nc3cc(C(=O)N4CCc5ccc(F)cc54)ccc3o2)C(=O)N1] |
| 107 | 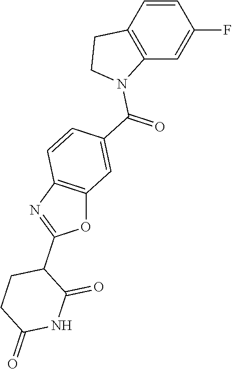 [SMILES: O=C1CCC(c2nc3ccc(C(=O)N4CCc5ccc(F)cc54)cc3o2)C(=O)N1] |
| 108 | 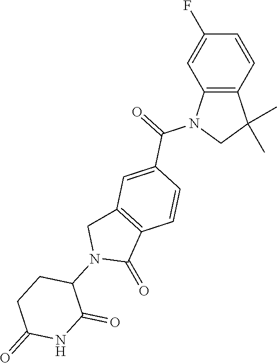 [SMILES: CC1(C)CN(C(=O)c2ccc3c(c2)CN(C2CCC(=O)NC2=O)C3=O)c2cc(F)ccc21] |
| 109 | 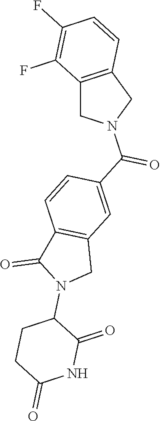 [SMILES: O=C1CCC(N2Cc3cc(C(=O)N4Cc5ccc(F)c(F)c5C4)ccc3C2=O)C(=O)N1] |
| 110 | 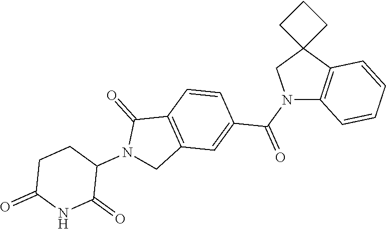 [SMILES: O=C1CCC(N2Cc3cc(C(=O)N4CC5(CCC5)c5ccccc54)ccc3C2=O)C(=O)N1] |
| 111 | 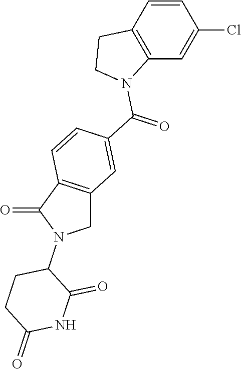 [SMILES: O=C1CCC(N2Cc3cc(C(=O)N4CCc5ccc(Cl)cc54)ccc3C2=O)C(=O)N1] |
| 112 | 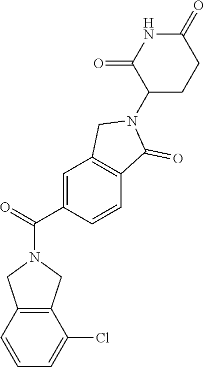 [SMILES: O=C1CCC(N2Cc3cc(C(=O)N4Cc5cccc(Cl)c5C4)ccc3C2=O)C(=O)N1] |
| 115 | 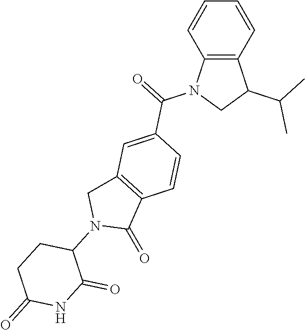 [SMILES: CC(C)C1CN(C(=O)c2ccc3c(c2)CN(C2CCC(=O)NC2=O)C3=O)c2ccccc21] |
| 116 | 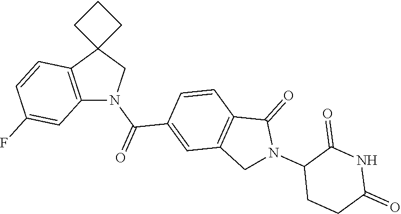 [SMILES: O=C1CCC(N2Cc3cc(C(=O)N4CC5(CCC5)c5ccc(F)cc54)ccc3C2=O)C(=O)N1] |
| 118 | 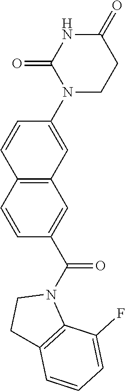 [SMILES: O=C1CCN(c2ccc3ccc(C(=O)N4CCc5cccc(F)c54)cc3c2)C(=O)N1] |
| 119 | 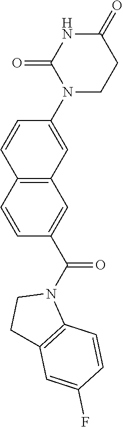 [SMILES: O=C1CCN(c2ccc3ccc(C(=O)N4CCc5cc(F)ccc54)cc3c2)C(=O)N1] |
| 120 | 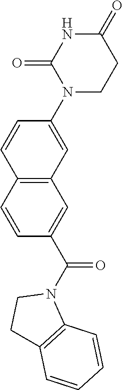 [SMILES: O=C1CCN(c2ccc3ccc(C(=O)N4CCc5ccccc54)cc3c2)C(=O)N1] |
| 123 | 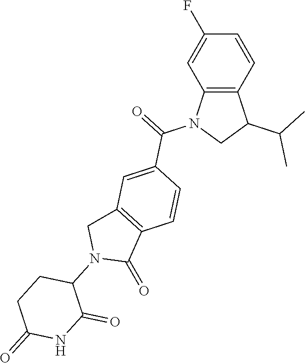 [SMILES: CC(C)C1CN(C(=O)c2ccc3c(c2)CN(C2CCC(=O)NC2=O)C3=O)c2cc(F)ccc21] |
| 124 | 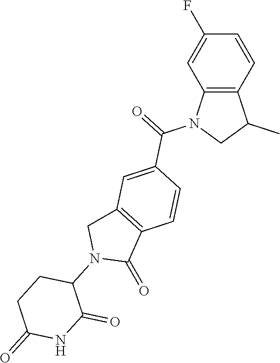 [SMILES: CC1CN(C(=O)c2ccc3c(c2)CN(C2CCC(=O)NC2=O)C3=O)c2cc(F)ccc21] |
| 125 | 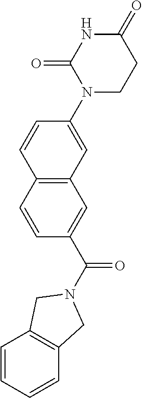 [SMILES: O=C1CCN(c2ccc3ccc(C(=O)N4Cc5ccccc5C4)cc3c2)C(=O)N1] |
| 126 | 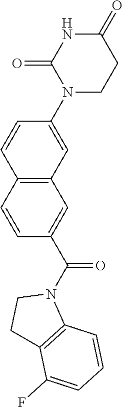 [SMILES: O=C1CCN(c2ccc3ccc(C(=O)N4CCc5c(F)cccc54)cc3c2)C(=O)N1] |
| 127 | 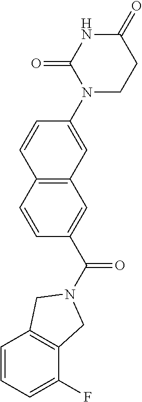 [SMILES: O=C1CCN(c2ccc3ccc(C(=O)N4Cc5cccc(F)c5C4)cc3c2)C(=O)N1] |
| 128 | 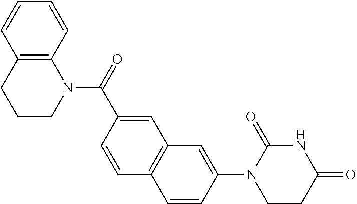 [SMILES: O=C1CCN(c2ccc3ccc(C(=O)N4CCCc5ccccc54)cc3c2)C(=O)N1] |
| 129 | 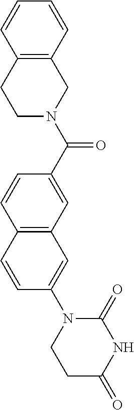 [SMILES: O=C1CCN(c2ccc3ccc(C(=O)N4CCc5ccccc5C4)cc3c2)C(=O)N1] |
| 130 | 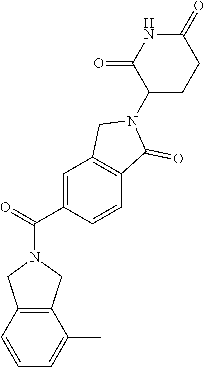 [SMILES: Cc1cccc2c1CN(C(=O)c1ccc3c(c1)CN(C1CCC(=O)NC1=O)C3=O)C2] |
| 131 | 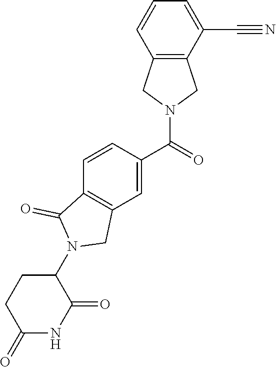 [SMILES: N#Cc1cccc2c1CN(C(=O)c1ccc3c(c1)CN(C1CCC(=O)NC1=O)C3=O)C2] |
| 132 | 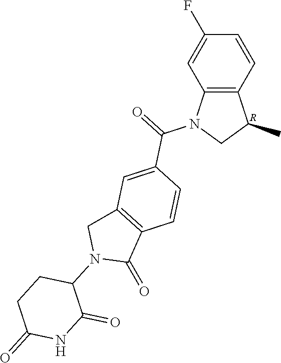 |
| 133 | 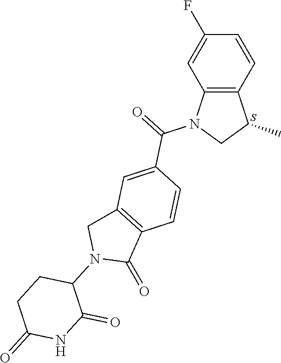 |
| 134 | 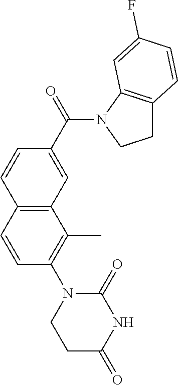 [SMILES: Cc1c(N2CCC(=O)NC2=O)ccc2ccc(C(=O)N3CCc4ccc(F)cc43)cc12] |
| 135 | 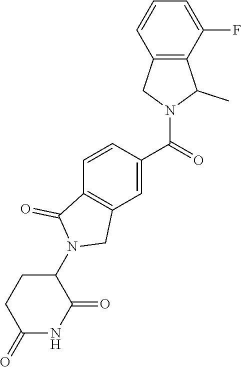 [SMILES: CC1c2c(F)cccc2CN1C(=O)c1ccc2c(c1)CN(C1CCC(=O)NC1=O)C2=O] |
| 136 | 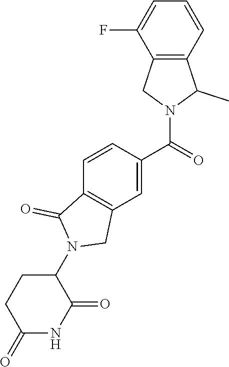 [SMILES: CC1c2cccc(F)c2CN1C(=O)c1ccc2c(c1)CN(C1CCC(=O)NC1=O)C2=O] |
| 137 | 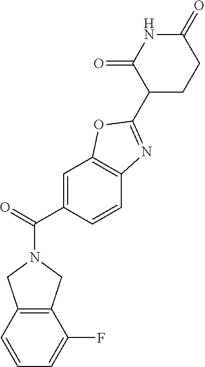 [SMILES: O=C1CCC(c2nc3ccc(C(=O)N4Cc5cccc(F)c5C4)cc3o2)C(=O)N1] |
| 139 | 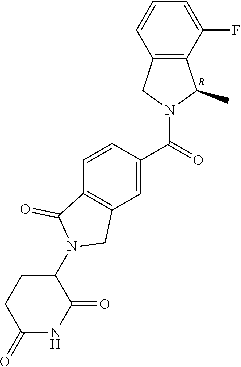 |
| 140 | 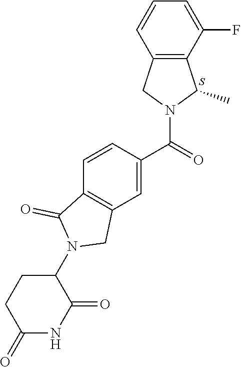 |
| 141 | 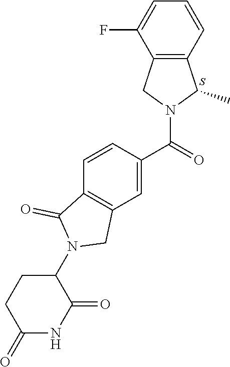 |
| 142 | 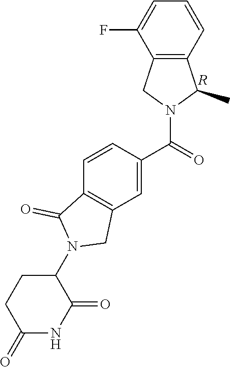 |
| 144 | 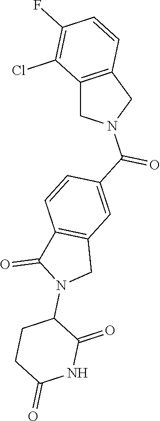 [SMILES: O=C1CCC(N2Cc3cc(C(=O)N4Cc5ccc(F)c(Cl)c5C4)ccc3C2=O)C(=O)N1] |
| 146 | 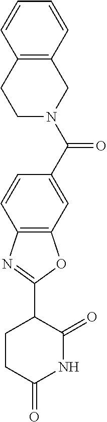 [SMILES: O=C1CCC(c2nc3ccc(C(=O)N4CCc5ccccc5C4)cc3o2)C(=O)N1] |
| 147 | 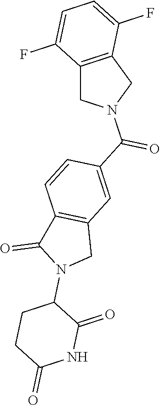 [SMILES: O=C1CCC(N2Cc3cc(C(=O)N4Cc5c(F)ccc(F)c5C4)ccc3C2=O)C(=O)N1] |
| 148 | 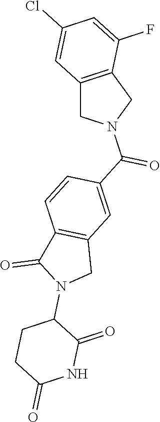 [SMILES: O=C1CCC(N2Cc3cc(C(=O)N4Cc5cc(Cl)cc(F)c5C4)ccc3C2=O)C(=O)N1] |
| 149 | 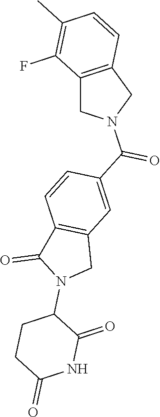 [SMILES: Cc1ccc2c(c1F)CN(C(=O)c1ccc3c(c1)CN(C1CCC(=O)NC1=O)C3=O)C2] |
| 150 | 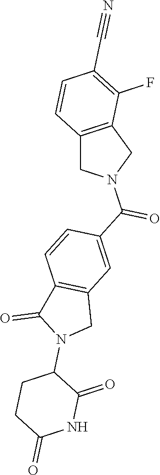 [SMILES: N#Cc1ccc2c(c1F)CN(C(=O)c1ccc3c(c1)CN(C1CCC(=O)NC1=O)C3=O)C2] |
| 151 | 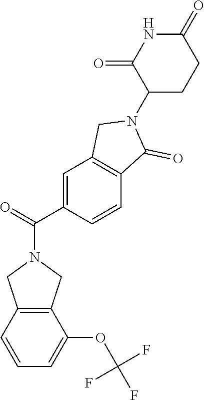 [SMILES: O=C1CCC(N2Cc3cc(C(=O)N4Cc5cccc(OC(F)(F)F)c5C4)ccc3C2=O)C(=O)N1] |
| 152 | 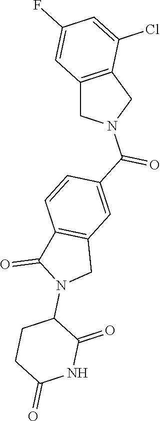 [SMILES: O=C1CCC(N2Cc3cc(C(=O)N4Cc5cc(F)cc(Cl)c5C4)ccc3C2=O)C(=O)N1] |
| 153 | 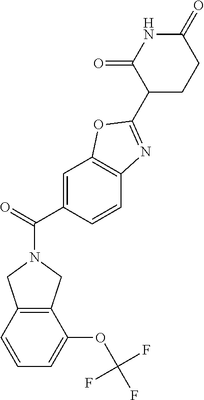 [SMILES: O=C1CCC(c2nc3ccc(C(=O)N4Cc5cccc(OC(F)(F)F)c5C4)cc3o2)C(=O)N1] |
| 154 | 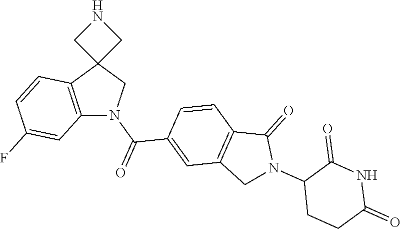 [SMILES: O=C1CCC(N2Cc3cc(C(=O)N4CC5(CNC5)c5ccc(F)cc54)ccc3C2=O)C(=O)N1] |
| 155 | 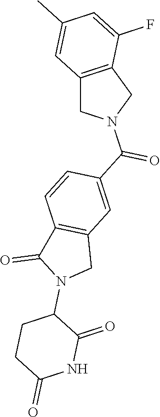 [SMILES: Cc1cc(F)c2c(c1)CN(C(=O)c1ccc3c(c1)CN(C1CCC(=O)NC1=O)C3=O)C2] |
| 156 | 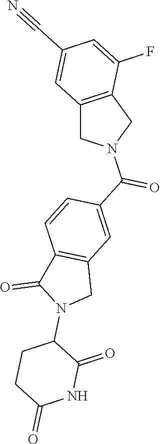 [SMILES: N#Cc1cc(F)c2c(c1)CN(C(=O)c1ccc3c(c1)CN(C1CCC(=O)NC1=O)C3=O)C2] |
| 158 | 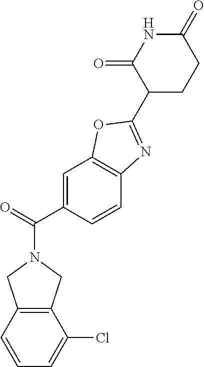 [SMILES: O=C1CCC(c2nc3ccc(C(=O)N4Cc5cccc(Cl)c5C4)cc3o2)C(=O)N1] |
| 159 | 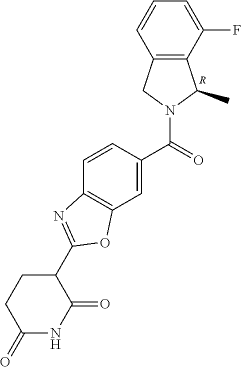 |
| 160 | 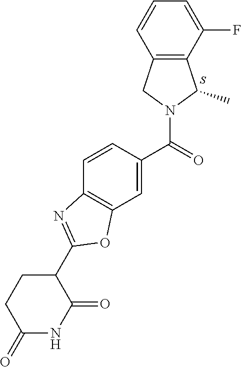 |
| 161 | 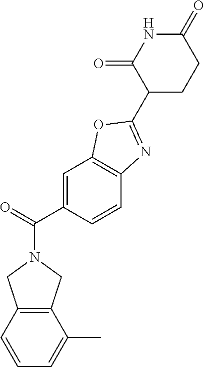 [SMILES: Cc1cccc2c1CN(C(=O)c1ccc3nc(C4CCC(=O)NC4=O)oc3c1)C2] |
| 162 | 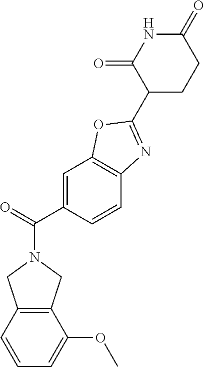 [SMILES: COc1cccc2c1CN(C(=O)c1ccc3nc(C4CCC(=O)NC4=O)oc3c1)C2] |
| 166 | 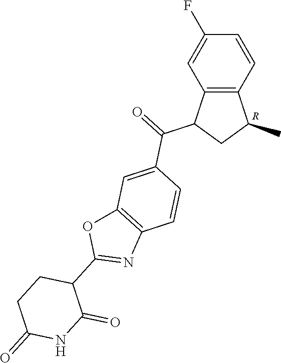 |
| 167 | 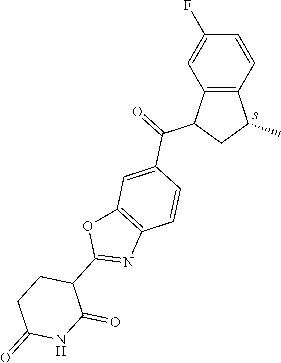 |
| 171 | 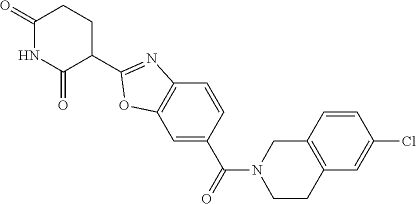 [SMILES: O=C1CCC(c2nc3ccc(C(=O)N4CCc5cc(Cl)ccc5C4)cc3o2)C(=O)N1] |
| 172 | 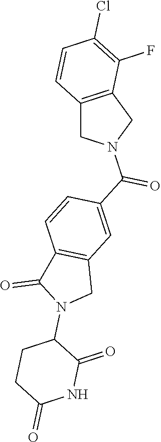 [SMILES: O=C1CCC(N2Cc3cc(C(=O)N4Cc5ccc(Cl)c(F)c5C4)ccc3C2=O)C(=O)N1] |
| 173 | 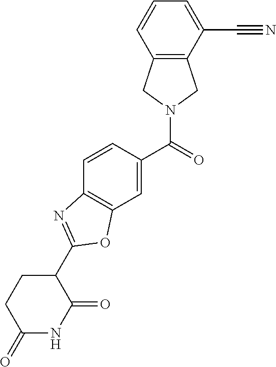 [SMILES: N#Cc1cccc2c1CN(C(=O)c1ccc3nc(C4CCC(=O)NC4=O)oc3c1)C2] |
| 174 | 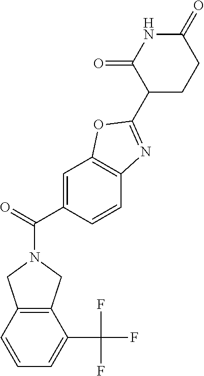 [SMILES: O=C1CCC(c2nc3ccc(C(=O)N4Cc5cccc(C(F)(F)F)c5C4)cc3o2)C(=O)N1] |
| 175 | 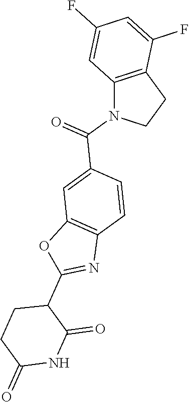 [SMILES: O=C1CCC(c2nc3ccc(C(=O)N4CCc5c(F)cc(F)cc54)cc3o2)C(=O)N1] |
| 176 | 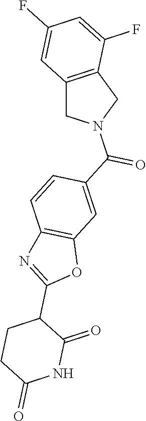 [SMILES: O=C1CCC(c2nc3ccc(C(=O)N4Cc5cc(F)cc(F)c5C4)cc3o2)C(=O)N1] |
| 177 | 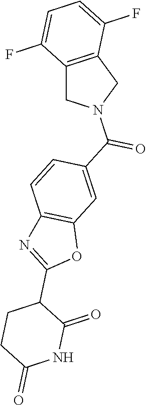 [SMILES: O=C1CCC(c2nc3ccc(C(=O)N4Cc5c(F)ccc(F)c5C4)cc3o2)C(=O)N1] |
| 178 | 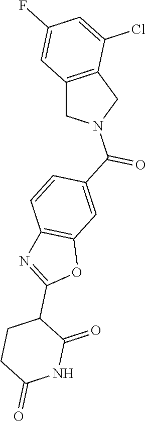 [SMILES: O=C1CCC(c2nc3ccc(C(=O)N4Cc5cc(F)cc(Cl)c5C4)cc3o2)C(=O)N1] |
| 179 | 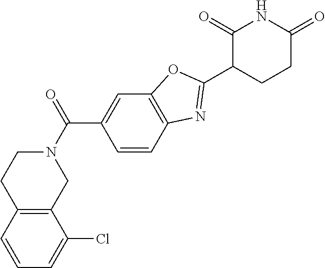 [SMILES: O=C1CCC(c2nc3ccc(C(=O)N4CCc5cccc(Cl)c5C4)cc3o2)C(=O)N1] |
| 180 | 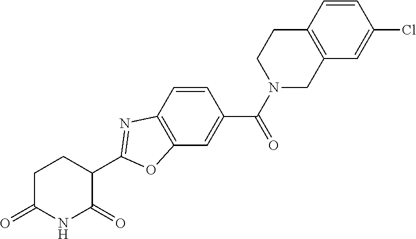 [SMILES: O=C1CCC(c2nc3ccc(C(=O)N4CCc5ccc(Cl)cc5C4)cc3o2)C(=O)N1] |
| 181 | 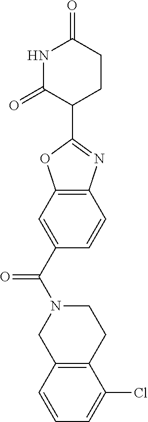 [SMILES: O=C1CCC(c2nc3ccc(C(=O)N4CCc5c(Cl)cccc5C4)cc3o2)C(=O)N1] |
| 182 | 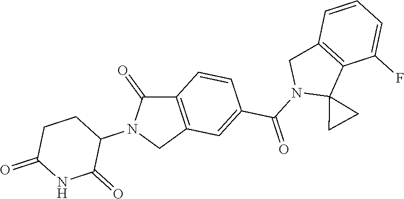 [SMILES: O=C1CCC(N2Cc3cc(C(=O)N4Cc5cccc(F)c5C45CC5)ccc3C2=O)C(=O)N1] |
| 185 | 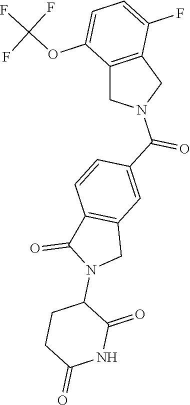 [SMILES: O=C1CCC(N2Cc3cc(C(=O)N4Cc5c(F)ccc(OC(F)(F)F)c5C4)ccc3C2=O)C(=O)N1] |
| 197 | 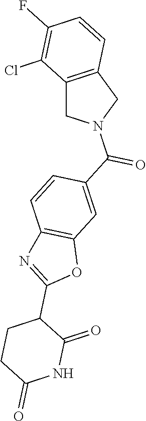 [SMILES: O=C1CCC(c2nc3ccc(C(=O)N4Cc5ccc(F)c(Cl)c5C4)cc3o2)C(=O)N1] |
| 199 | 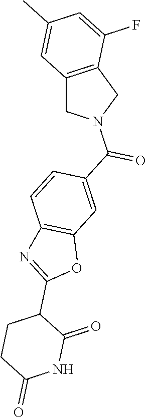 [SMILES: Cc1cc(F)c2c(c1)CN(C(=O)c1ccc3nc(C4CCC(=O)NC4=O)oc3c1)C2] |
| 200 | 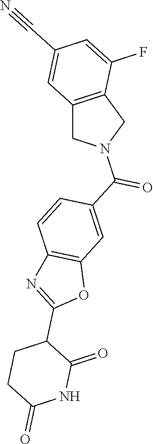 [SMILES: N#Cc1cc(F)c2c(c1)CN(C(=O)c1ccc3nc(C4CCC(=O)NC4=O)oc3c1)C2] |
| 210 | 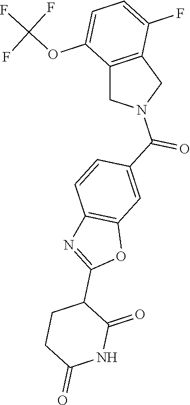 [SMILES: O=C1CCC(c2nc3ccc(C(=O)N4Cc5c(F)ccc(OC(F)(F)F)c5C4)cc3o2)C(=O)N1] |
| 211 | 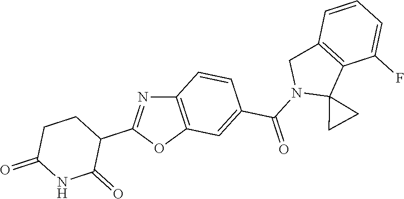 [SMILES: O=C1CCC(c2nc3ccc(C(=O)N4Cc5cccc(F)c5C45CC5)cc3o2)C(=O)N1] |
| 261 | 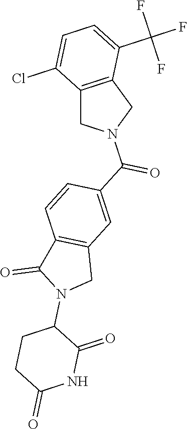 [SMILES: O=C1CCC(N2Cc3cc(C(=O)N4Cc5c(Cl)ccc(C(F)(F)F)c5C4)ccc3C2=O)C(=O)N1] |
| 263 | 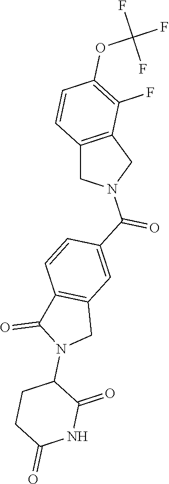 [SMILES: O=C1CCC(N2Cc3cc(C(=O)N4Cc5ccc(OC(F)(F)F)c(F)c5C4)ccc3C2=O)C(=O)N1] |
| 264 | 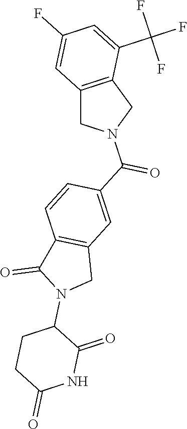 [SMILES: O=C1CCC(N2Cc3cc(C(=O)N4Cc5cc(F)cc(C(F)(F)F)c5C4)ccc3C2=O)C(=O)N1] [symbol] |

[symbol]

indicates data missing or illegible when filed

In some embodiment, the has the structure of Formula (I-c):

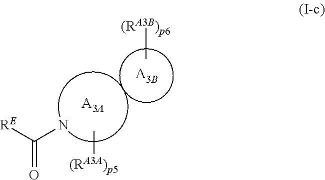

In some embodiment, each Ring A_{3A} and Ring A_{3B} is monocyclic ring selected from 4-7 membered saturated or partially unsaturated ring having 0-4 heteroatoms, in addition to the nitrogen already depicted in Ring A_{3A}, independently selected from nitrogen, oxygen, and sulfur.

In some embodiment, 0-4 methylene moieties (—CH₂—) contained in the Ring A_{3A} or Ring A_{3B} may be independently replaced with oxo (—C═O), —NH—, —N—C₁₋₃ alkyl-, or —O—.

In some embodiment, Ring A_{3A} and Ring A_{3B} taken together form a spiro-heterocyclic with one shared/border carbon atom between Ring A_{3A} and Ring A_{3B}.

In some embodiment, each R^{A3A} and R^{A3B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A3A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{A3B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript p5 and p6 is independently 0, 1, 2, 3, 4, 5, 6, 7, 8, 9, or 10.

In some embodiment, the compound may comprise additional substituent of an optionally substituted ring of Ring A_{3C} as depicted in the Formula (I-c-1) below:

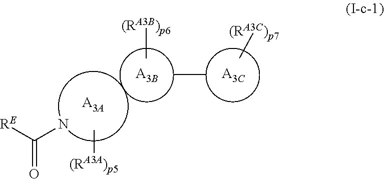

In some embodiment, Ring A_{3C} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur.

In some embodiment, 0-3 methylene moieties (—CH₂—) contained in the Ring A_{3C} may be independently replaced with oxo (—C═O), —NH—, —N—C₁₋₃ alkyl-, or —O—.

In some embodiment, R^{A3C} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R′)(R^{b}), -alkyl-OC(O)N(R^{a})(R^{b}), -alkyl-N(R^{a})C(O)R^{a}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A3C} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₅-C₁₃ spiroalkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript p7 is 0, 1, 2, 3, 4, 5, 6, 7, 8, 9, or 10.

In some embodiment, the compound has the structure of Formula (I-c-2) or (I-c-3):

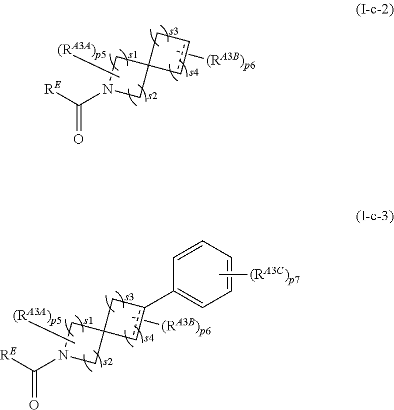

In some embodiment, each subscript s1, s2, s3 and s4 is independently 1 or 2.

In some embodiment, a spiro-heterocyclic ring containing the nitrogen already depicted in Formula (I-c-2) or (I-c-3) above, optionally comprises one double bond therein depicted as dotted line, and 0-2 methylene moieties (—CH₂—) contained in the ring may be replaced with oxo (—C═O), —NH—, —N—C₁₋₃ alkyl-, —O—.

In some embodiment, each R^{A3A}, R^{A3B} and R^{A3C} is independently hydrogen, deuterium, halogen, C₁-C₄ alkyl (i.e., straight or branched), C₁-C₃ alkoxy, C₁-C₃ haloalkyl, C₁-C₃ haloalkoxy, or cyano.

In some embodiment, the compound has the structure selected from Formula (I-c-4) to Formula (I-b-14):

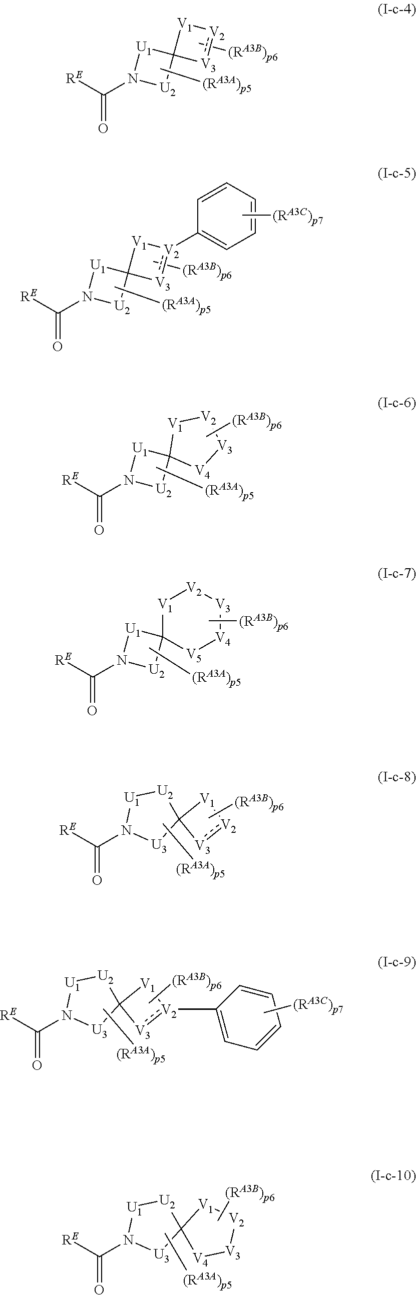

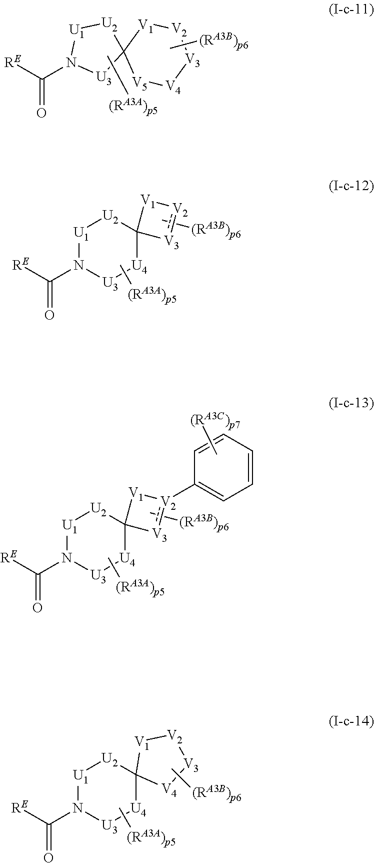

In some embodiment, each of U₁, U₂, U₃ and U₄ is independently C═O, O, NH, N—C₁₋₃ alkyl or CH₂.

In some embodiment, each of V₁, V₂, V₃, V₄ and V₅ is independently C═O, O, NH, N—C₁₋₃ alkyl or CH₂.

In some embodiment, a spiro-heterocyclic ring containing the nitrogen already depicted in Formula (I-c-4) to (I-c-14) above, optionally comprises one double bond therein depicted as dotted line.

In some embodiment, the compound of the present disclosure is selected from the following [Table A-4].

TABLE A-4

| Example Number | Structure |
|---|---|
| 113 | 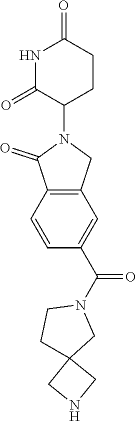 [SMILES: O=C1CCC(N2Cc3cc(C(=O)N4CCC5(CNC5)C4)ccc3C2=O)C(=O)N1] |
| 114 | 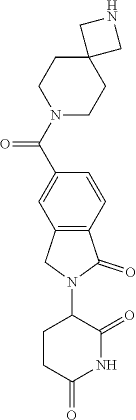 [SMILES: O=C1CCC(N2Cc3cc(C(=O)N4CCC5(CC4)CNC5)ccc3C2=O)C(=O)N1] |
| 117 | 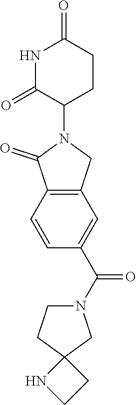 [SMILES: O=C1CCC(N2Cc3cc(C(=O)N4CCC5(CCN5)C4)ccc3C2=O)C(=O)N1] |
| 121 | 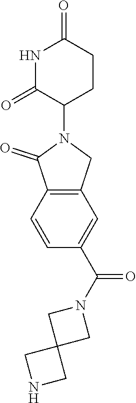 [SMILES: O=C1CCC(N2Cc3cc(C(=O)N4CC5(CNC5)C4)ccc3C2=O)C(=O)N1] |
| 122 | 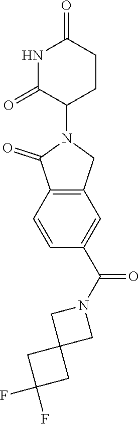 [SMILES: O=C1CCC(N2Cc3cc(C(=O)N4CC5(C4)CC(F)(F)C5)ccc3C2=O)C(=O)N1] |
| 138 | 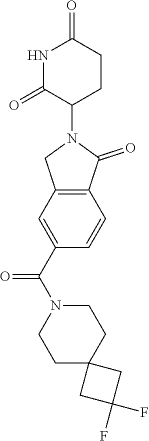 [SMILES: O=C1CCC(N2Cc3cc(C(=O)N4CCC5(CC4)CC(F)(F)C5)ccc3C2=O)C(=O)N1] |
| 145 | 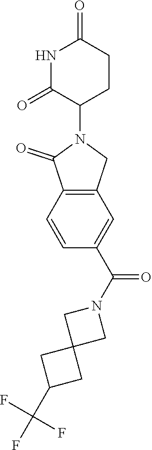 [SMILES: O=C1CCC(N2Cc3cc(C(=O)N4CC5(CC(C(F)(F)F)C5)C4)ccc3C2=O)C(=O)N1] |
| 157 | 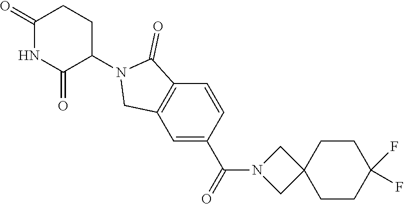 [SMILES: O=C1CCC(N2Cc3cc(C(=O)N4CC5(CCC(F)(F)CC5)C4)ccc3C2=O)C(=O)N1] |
| 163 | 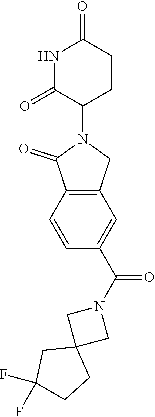 [SMILES: O=C1CCC(N2Cc3cc(C(=O)N4CC5(CCC(F)(F)C5)C4)ccc3C2=O)C(=O)N1] |
| 165 | 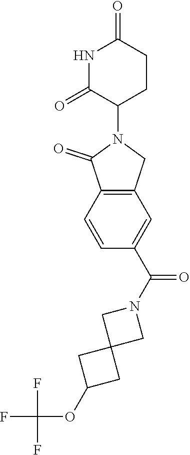 [SMILES: O=C1CCC(N2Cc3cc(C(=O)N4CC5(CC(OC(F)(F)F)C5)C4)ccc3C2=O)C(=O)N1] |
| 168 | 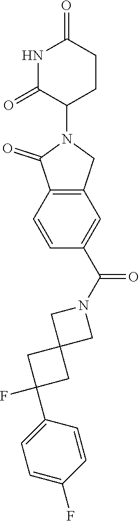 [SMILES: O=C1CCC(N2Cc3cc(C(=O)N4CC5(C4)CC(F)(c4ccc(F)cc4)C5)ccc3C2=O)C(=O)N1] |
| 169 | 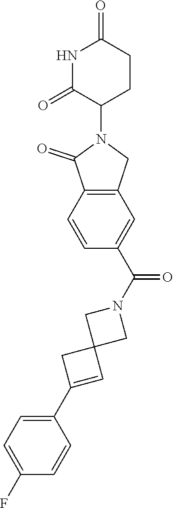 [SMILES: O=C1CCC(N2Cc3cc(C(=O)N4CC5(C=C(c6ccc(F)cc6)C5)C4)ccc3C2=O)C(=O)N1] |
| 183 | 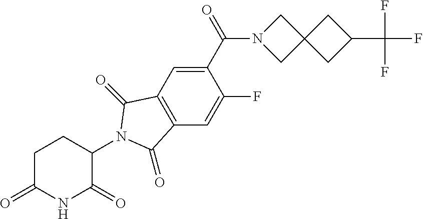 [SMILES: O=C1CCC(N2C(=O)c3cc(F)c(C(=O)N4CC5(CC(C(F)(F)F)C5)C4)cc3C2=O)C(=O)N1] |
| 184 | 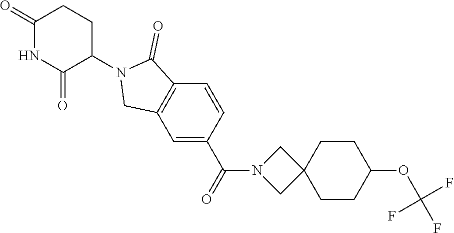 [SMILES: O=C1CCC(N2Cc3cc(C(=O)N4CC5(CCC(OC(F)(F)F)CC5)C4)ccc3C2=O)C(=O)N1] |
| 186 | 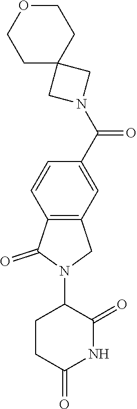 [SMILES: O=C1CCC(N2Cc3cc(C(=O)N4CC5(CCOCC5)C4)ccc3C2=O)C(=O)N1] |
| 187 | 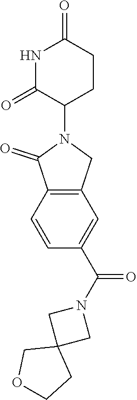 [SMILES: O=C1CCC(N2Cc3cc(C(=O)N4CC5(CCOC5)C4)ccc3C2=O)C(=O)N1] |
| 188 | 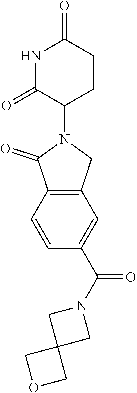 [SMILES: O=C1CCC(N2Cc3cc(C(=O)N4CC5(COC5)C4)ccc3C2=O)C(=O)N1] |
| 189 | 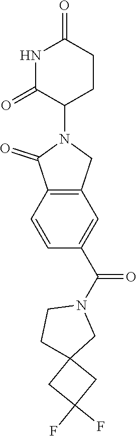 [SMILES: O=C1CCC(N2Cc3cc(C(=O)N4CCC5(C4)CC(F)(F)C5)ccc3C2=O)C(=O)N1] |
| 190 | 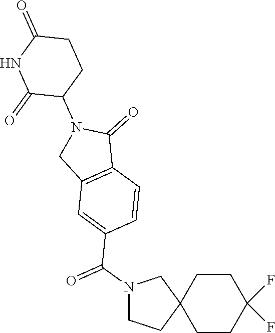 [SMILES: O=C1CCC(N2Cc3cc(C(=O)N4CCC5(CCC(F)(F)CC5)C4)ccc3C2=O)C(=O)N1] |
| 195 | 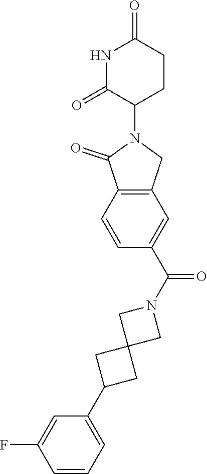 [SMILES: O=C1CCC(N2Cc3cc(C(=O)N4CC5(CC(c6cccc(F)c6)C5)C4)ccc3C2=O)C(=O)N1] |
| 196 | 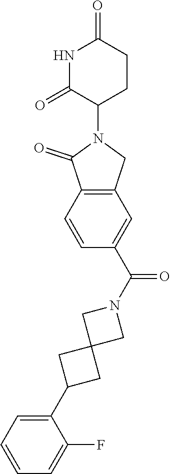 [SMILES: O=C1CCC(N2Cc3cc(C(=O)N4CC5(CC(c6ccccc6F)C5)C4)ccc3C2=O)C(=O)N1] |
| 198 | 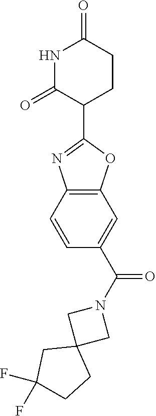 [SMILES: O=C1CCC(c2nc3ccc(C(=O)N4CC5(CCC(F)(F)C5)C4)cc3o2)C(=O)N1] |
| 201 | 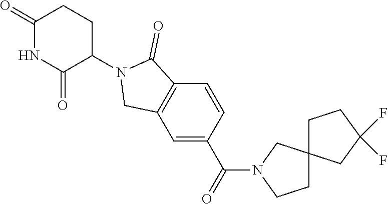 [SMILES: O=C1CCC(N2Cc3cc(C(=O)N4CCC5(CCC(F)(F)C5)C4)ccc3C2=O)C(=O)N1] |
| 203 | 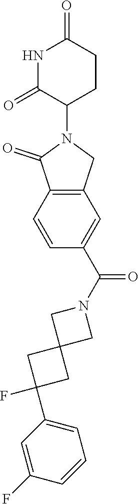 [SMILES: O=C1CCC(N2Cc3cc(C(=O)N4CC5(C4)CC(F)(c4cccc(F)c4)C5)ccc3C2=O)C(=O)N1] |
| 204 | 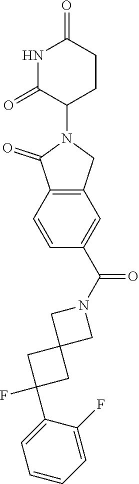 [SMILES: O=C1CCC(N2Cc3cc(C(=O)N4CC5(C4)CC(F)(c4ccccc4F)C5)ccc3C2=O)C(=O)N1] |
| 205 | 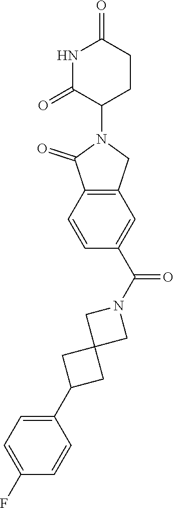 [SMILES: O=C1CCC(N2Cc3cc(C(=O)N4CC5(CC(c6ccc(F)cc6)C5)C4)ccc3C2=O)C(=O)N1] |
| 206 | 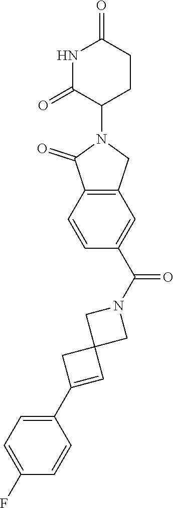 [SMILES: O=C1CCC(N2Cc3cc(C(=O)N4CC5(C=C(c6ccc(F)cc6)C5)C4)ccc3C2=O)C(=O)N1] |
| 207 | 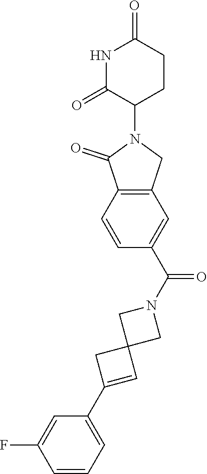 [SMILES: O=C1CCC(N2Cc3cc(C(=O)N4CC5(C=C(c6cccc(F)c6)C5)C4)ccc3C2=O)C(=O)N1] |
| 208 | 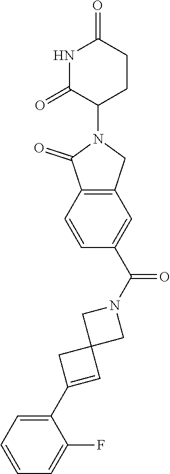 [SMILES: O=C1CCC(N2Cc3cc(C(=O)N4CC5(C=C(c6ccccc6F)C5)C4)ccc3C2=O)C(=O)N1] |
| 209 | 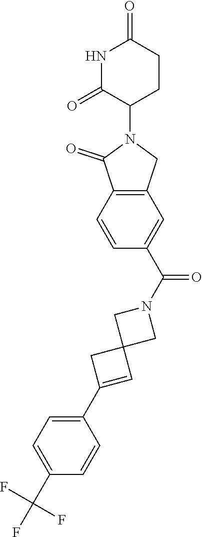 [SMILES: O=C1CCC(N2Cc3cc(C(=O)N4CC5(C=C(c6ccc(C(F)(F)F)cc6)C5)C4)ccc3C2=O)C(=O)N1] |
| 220 | 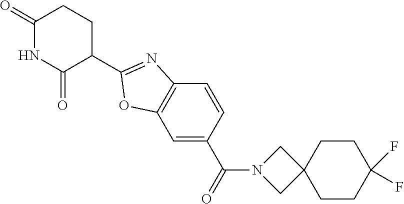 [SMILES: O=C1CCC(c2nc3ccc(C(=O)N4CC5(CCC(F)(F)CC5)C4)cc3o2)C(=O)N1] |
| 221 | 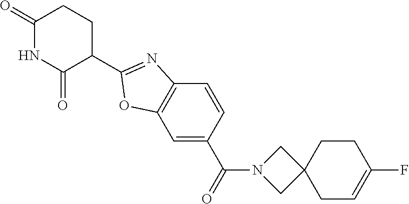 [SMILES: O=C1CCC(c2nc3ccc(C(=O)N4CC5(CC=C(F)CC5)C4)cc3o2)C(=O)N1] |
| 222 | 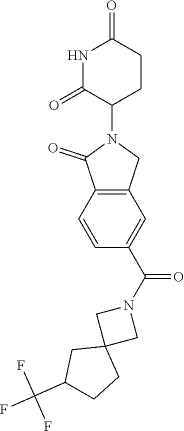 [SMILES: O=C1CCC(N2Cc3cc(C(=O)N4CC5(CCC(C(F)(F)F)C5)C4)ccc3C2=O)C(=O)N1] |
| 228 | 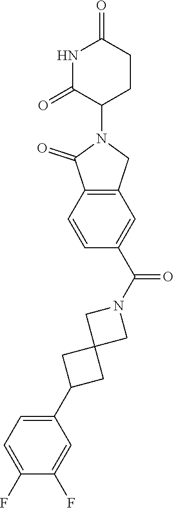 [SMILES: O=C1CCC(N2Cc3cc(C(=O)N4CC5(CC(c6ccc(F)c(F)c6)C5)C4)ccc3C2=O)C(=O)N1] |
| 229 | 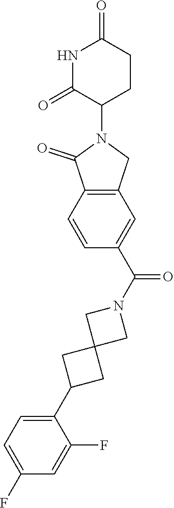 [SMILES: O=C1CCC(N2Cc3cc(C(=O)N4CC5(CC(c6ccc(F)cc6F)C5)C4)ccc3C2=O)C(=O)N1] |
| 230 | 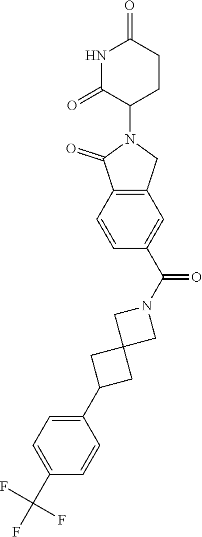 [SMILES: O=C1CCC(N2Cc3cc(C(=O)N4CC5(CC(c6ccc(C(F)(F)F)cc6)C5)C4)ccc3C2=O)C(=O)N1] |
| 231 | 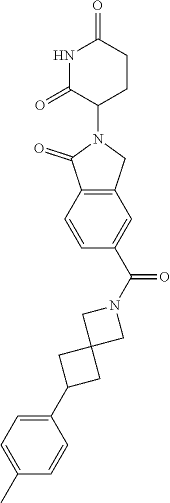 [SMILES: Cc1ccc(C2CC3(C2)CN(C(=O)c2ccc4c(c2)CN(C2CCC(=O)NC2=O)C4=O)C3)cc1] |
| 232 | 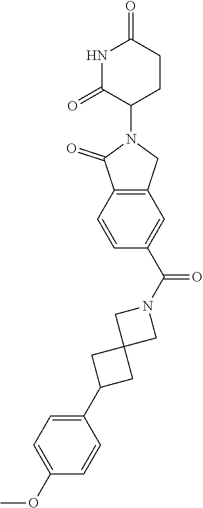 [SMILES: COc1ccc(C2CC3(C2)CN(C(=O)c2ccc4c(c2)CN(C2CCC(=O)NC2=O)C4=O)C3)cc1] |
| 233 | 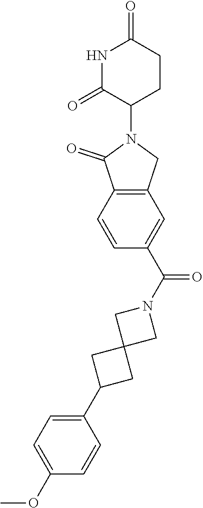 [SMILES: COc1ccc(C2CC3(C2)CN(C(=O)c2ccc4c(c2)CN(C2CCC(=O)NC2=O)C4=O)C3)cc1] |
| 234 | 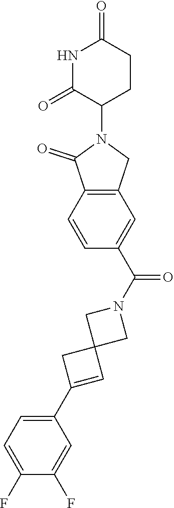 [SMILES: O=C1CCC(N2Cc3cc(C(=O)N4CC5(C=C(c6ccc(F)c(F)c6)C5)C4)ccc3C2=O)C(=O)N1] |
| 235 | 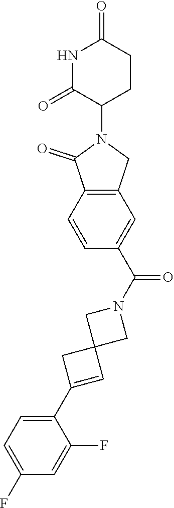 [SMILES: O=C1CCC(N2Cc3cc(C(=O)N4CC5(C=C(c6ccc(F)cc6F)C5)C4)ccc3C2=O)C(=O)N1] |
| 236 | 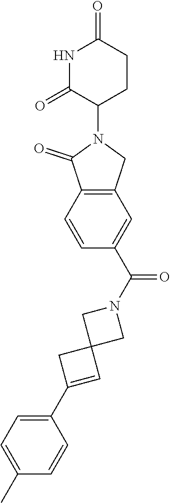 [SMILES: Cc1ccc(C2=CC3(C2)CN(C(=O)c2ccc4c(c2)CN(C2CCC(=O)NC2=O)C4=O)C3)cc1] |
| 237 | 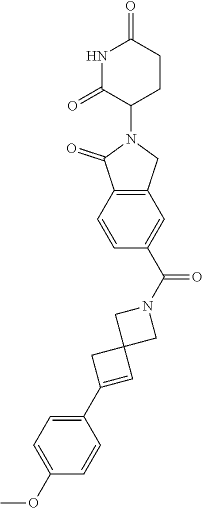 [SMILES: COc1ccc(C2=CC3(C2)CN(C(=O)c2ccc4c(c2)CN(C2CCC(=O)NC2=O)C4=O)C3)cc1] |
| 238 | 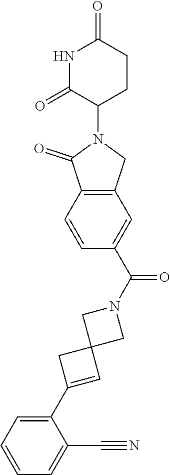 [SMILES: N#Cc1ccccc1C1=CC2(C1)CN(C(=O)c1ccc3c(c1)CN(C1CCC(=O)NC1=O)C3=O)C2] |
| 239 | 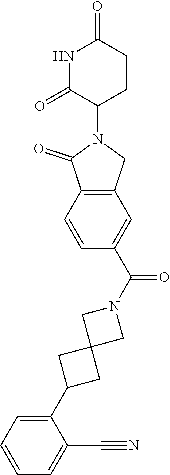 [SMILES: N#Cc1ccccc1C1CC2(C1)CN(C(=O)c1ccc3c(c1)CN(C1CCC(=O)NC1=O)C3=O)C2] |
| 240 | 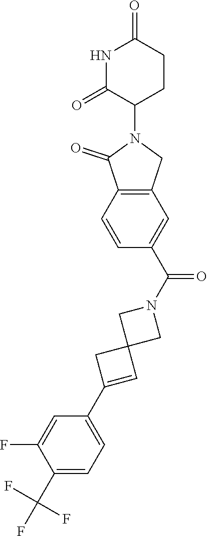 [SMILES: O=C1CCC(N2Cc3cc(C(=O)N4CC5(C=C(c6ccc(C(F)(F)F)c(F)c6)C5)C4)ccc3C2=O)C(=O)N1] |
| 241 | 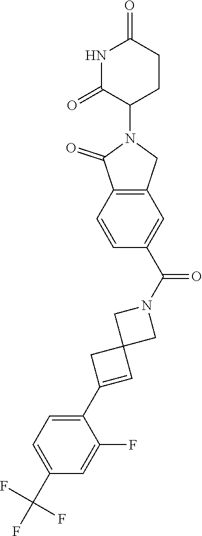 [SMILES: O=C1CCC(N2Cc3cc(C(=O)N4CC5(C=C(c6ccc(C(F)(F)F)cc6F)C5)C4)ccc3C2=O)C(=O)N1] |
| 242 | 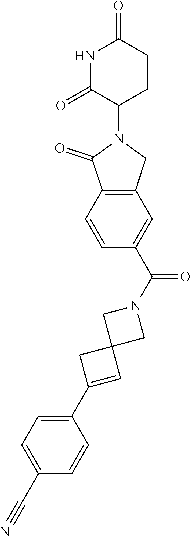 [SMILES: N#Cc1ccc(C2=CC3(C2)CN(C(=O)c2ccc4c(c2)CN(C2CCC(=O)NC2=O)C4=O)C3)cc1] |
| 243 | 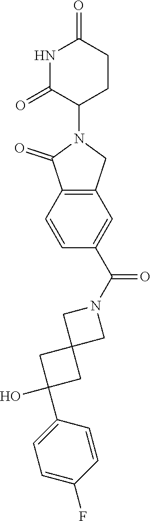 [SMILES: O=C1CCC(N2Cc3cc(C(=O)N4CC5(C4)CC(O)(c4ccc(F)cc4)C5)ccc3C2=O)C(=O)N1] |
| 244 | 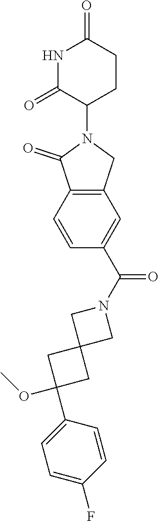 [SMILES: COC1(c2ccc(F)cc2)CC2(CN(C(=O)c3ccc4c(c3)CN(C3CCC(=O)NC3=O)C4=O)C2)C1] |
| 245 | 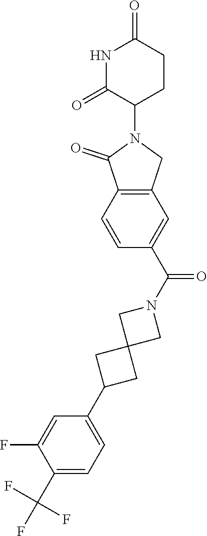 [SMILES: O=C1CCC(N2Cc3cc(C(=O)N4CC5(CC(c6ccc(C(F)(F)F)c(F)c6)C5)C4)ccc3C2=O)C(=O)N1] |
| 246 | 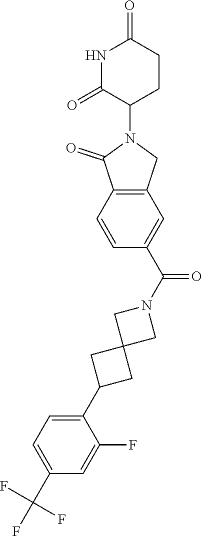 [SMILES: O=C1CCC(N2Cc3cc(C(=O)N4CC5(CC(c6ccc(C(F)(F)F)cc6F)C5)C4)ccc3C2=O)C(=O)N1] |
| 247 | 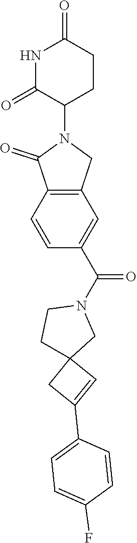 [SMILES: O=C1CCC(N2Cc3cc(C(=O)N4CCC5(C=C(c6ccc(F)cc6)C5)C4)ccc3C2=O)C(=O)N1] |
| 248 | 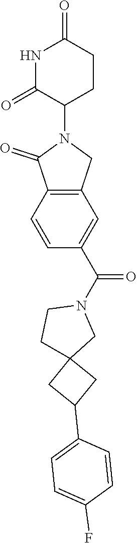 [SMILES: O=C1CCC(N2Cc3cc(C(=O)N4CCC5(CC(c6ccc(F)cc6)C5)C4)ccc3C2=O)C(=O)N1] |
| 249 | 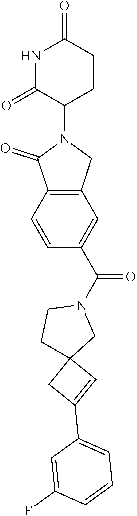 [SMILES: O=C1CCC(N2Cc3cc(C(=O)N4CCC5(C=C(c6cccc(F)c6)C5)C4)ccc3C2=O)C(=O)N1] |
| 250 | 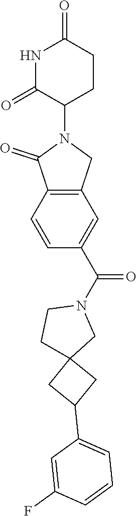 [SMILES: O=C1CCC(N2Cc3cc(C(=O)N4CCC5(CC(c6cccc(F)c6)C5)C4)ccc3C2=O)C(=O)N1] |
| 251 | 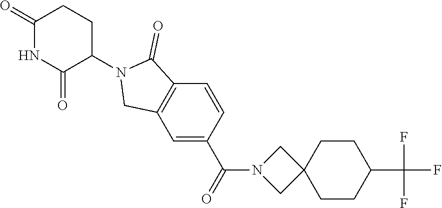 [SMILES: O=C1CCC(N2Cc3cc(C(=O)N4CC5(CCC(C(F)(F)F)CC5)C4)ccc3C2=O)C(=O)N1] |
| 252 | 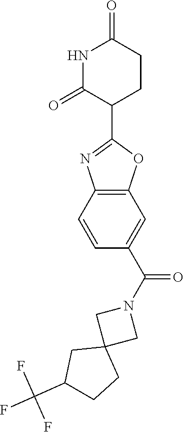 [SMILES: O=C1CCC(c2nc3ccc(C(=O)N4CC5(CCC(C(F)(F)F)C5)C4)cc3o2)C(=O)N1] |
| 259 | 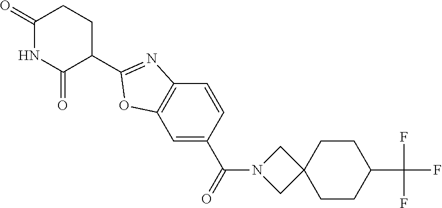 [SMILES: O=C1CCC(c2nc3ccc(C(=O)N4CC5(CCC(C(F)(F)F)CC5)C4)cc3o2)C(=O)N1] |
| 262 | 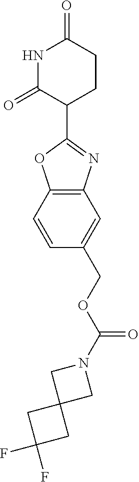 [SMILES: O=C1CCC(c2nc3cc(COC(=O)N4CC5(C4)CC(F)(F)C5)ccc3o2)C(=O)N1] |

In some embodiment, the compound has the structure of Formula (I-d):

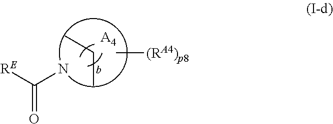

In some embodiment, Ring A₄ is fully saturated 5-10 membered bridged-heterocyclic ring having 0-3 heteroatoms, in addition to the nitrogen already depicted in Ring A₄, independently selected from nitrogen, oxygen, and sulfur.

In some embodiment, 0-3 methylene moieties (—CH₂—) contained in the Ring A₄ may be independently replaced with oxo (—C═O), —NH—, —N—C₁₋₃ alkyl-, or —O—.

In some embodiment, a subscript b is 1, 2 or 3.

In some embodiment, R^{A4} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R′)(R^{b}), -alkyl-OC(O)N(R^{a})(R^{b}), -alkyl-N(R^{a})C(O)R^{a}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{A4} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript p8 is 0, 1, 2, 3, 4, 5, 6, 7, 8, 9, or 10.

In some embodiment, the compound has the structure of Formula (I-d-1) or (I-d-2):

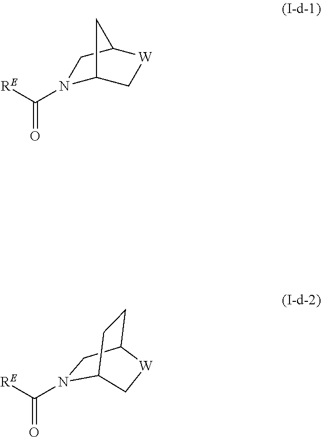

In some embodiment, the compound of the present disclosure is selected from the following [Table A-5].

TABLE A-5

| Example Number | Structure |
|---|---|
| 98 | 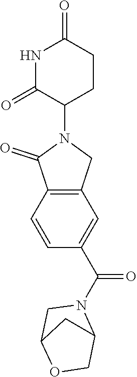 [SMILES: O=C1CCC(N2Cc3cc(C(=O)N4CC5CC4CO5)ccc3C2=O)C(=O)N1] |
| 99 | 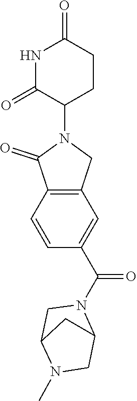 [SMILES: CN1CC2CC1CN2C(=O)c1ccc2c(c1)CN(C1CCC(=O)NC1=O)C2=O] |

The present disclosure provides novel compounds which are represented by Formula (II) as follows, or a pharmaceutically acceptable salt, enantiomer, stereoisomer or prodrug thereof.

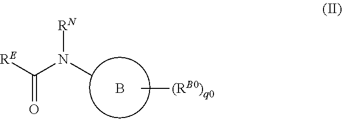

In some embodiment, R^{E} is selected from groups consisting of

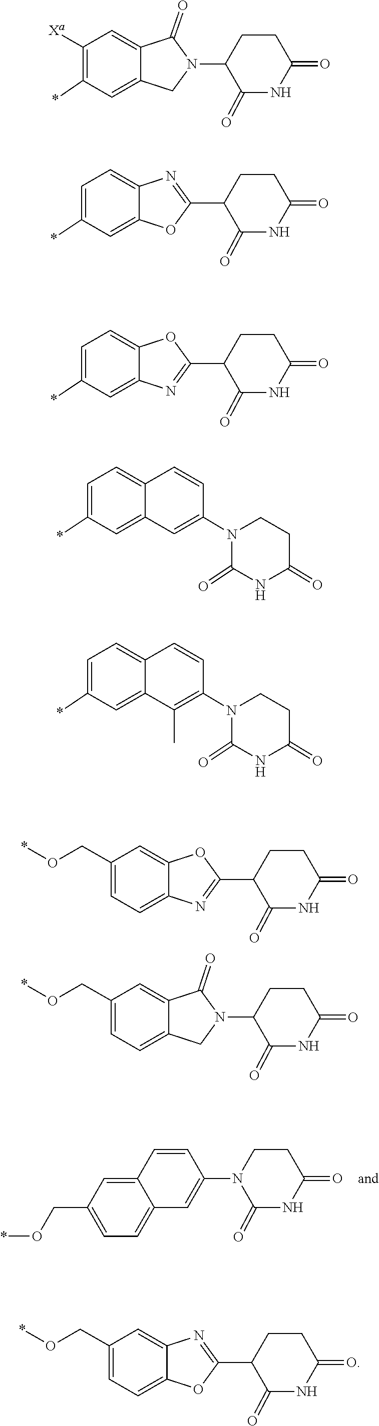

In some embodiment, X^{a} is H or F.

In some embodiment, R^{N} is hydrogen or C₁-C₃ alkyl.

In some embodiment, Ring B is a monocyclic or bicyclic ring (e.g., fused, spiro, bridged) selected from 4-12 membered saturated, partially unsaturated ring having 0-4 heteroatoms independently selected from nitrogen, oxygen, and sulfur.

In some embodiment, 0-4 methylene moieties (—CH₂—) contained in the Ring B may be replaced with oxo (—C═O), —NH—, —N—C₁₋₃ alkyl-, —O—.

In some embodiment, R^{B0} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, N(R^{b})SO₂R^{c}, or a monocyclic or bicyclic ring (e.g., fused, spiro, bridged) optionally substituted with one or more R^{d}.

In some embodiment, the monocyclic or bicyclic ring is selected from a 3-7 membered saturated or partially unsaturated monocyclic carbocyclic ring, a 7-11 membered saturated or partially unsaturated bicyclic carbocyclic ring, a 4-7 membered saturated or partially unsaturated monocyclic heterocyclic ring having 1-4 heteroatoms independently selected from nitrogen, oxygen, and sulfur, a 7-11 membered saturated or partially unsaturated bicyclic heterocyclic ring having 1-5 heteroatoms independently selected from nitrogen, oxygen, and sulfur, a benzene (i.e., phenyl) ring, a 8-10 membered bicyclic aryl ring, a 5-6 membered monocyclic heteroaryl ring having 1-4 heteroatoms independently selected from nitrogen, oxygen, and sulfur, and a 8-10 membered bicyclic heteroaryl ring having 1-5 heteroatoms independently selected from nitrogen, oxygen, and sulfur.

In some embodiment, two of R^{B0} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript q0 is 0, 1, 2, 3, 4, 5, 6, 7, 8, 9, or 10.

In some embodiment, each stereocenter in the compound of Formula above is independently the R-enantiomer, the S-enantiomer or a mixture of R- and S-enantiomers.

In some embodiment, each double bond in the compound of Formula above is independently cis or trans.

In some embodiment, the compound of the present disclosure is selected from the following [Table B-1].

TABLE B-1

| Example Number | Structure |
|---|---|
| 58 | 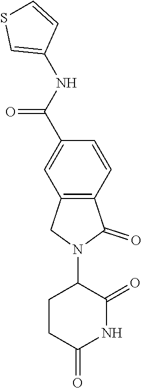 [SMILES: O=C1CCC(N2Cc3cc(C(=O)Nc4ccsc4)ccc3C2=O)C(=O)N1] |
| 90 | 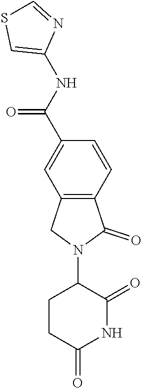 [SMILES: O=C1CCC(N2Cc3cc(C(=O)Nc4cscn4)ccc3C2=O)C(=O)N1] |
| 93 | 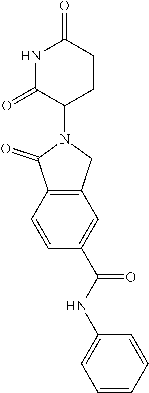 [SMILES: O=C1CCC(N2Cc3cc(C(=O)Nc4ccccc4)ccc3C2=O)C(=O)N1] |
| 94 | 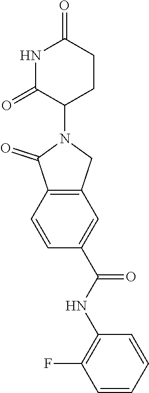 [SMILES: O=C1CCC(N2Cc3cc(C(=O)Nc4ccccc4F)ccc3C2=O)C(=O)N1] |
| 96 | 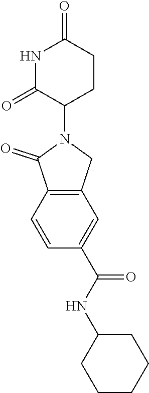 [SMILES: O=C1CCC(N2Cc3cc(C(=O)NC4CCCCC4)ccc3C2=O)C(=O)N1] |
| 97 | 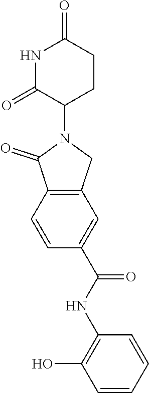 [SMILES: O=C1CCC(N2Cc3cc(C(=O)Nc4ccccc4O)ccc3C2=O)C(=O)N1] |
| 143 | 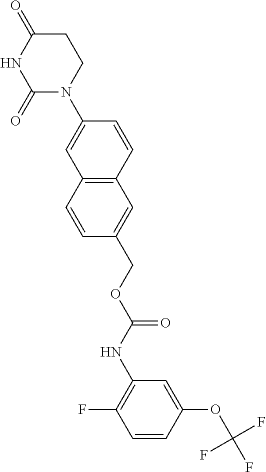 [SMILES: O=C1CCN(c2ccc3cc(COC(=O)Nc4cc(OC(F)(F)F)ccc4F)ccc3c2)C(=O)N1] |
| 164 | 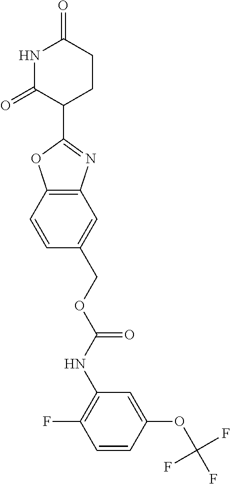 [SMILES: O=C1CCC(c2nc3cc(COC(=O)Nc4cc(OC(F)(F)F)ccc4F)ccc3o2)C(=O)N1] |
| 212 | 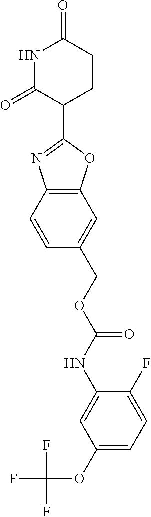 [SMILES: O=C1CCC(c2nc3ccc(COC(=O)Nc4cc(OC(F)(F)F)ccc4F)cc3o2)C(=O)N1] |
| 223 | 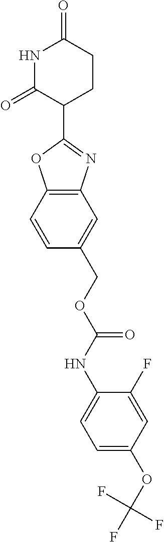 [SMILES: O=C1CCC(c2nc3cc(COC(=O)Nc4ccc(OC(F)(F)F)cc4F)ccc3o2)C(=O)N1] |
| 224 | 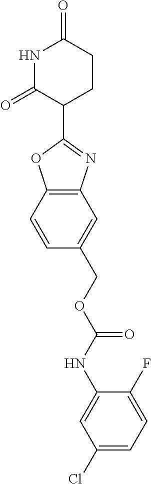 [SMILES: O=C1CCC(c2nc3cc(COC(=O)Nc4cc(Cl)ccc4F)ccc3o2)C(=O)N1] |

In some embodiment, the compound has the structure of Formula (II-b):

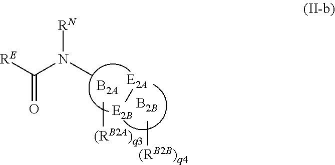

In some embodiment, Ring B_{2A} is a monocyclic heterocyclic ring selected from 3-7 membered saturated, partially unsaturated heterocyclic ring, or 5-6 membered heteroaryl ring having 0-2 heteroatoms independently selected from nitrogen, oxygen, and sulfur.

In some embodiment, 0-2 methylene moieties (—CH₂—) contained in the Ring B_{2A} may be independently replaced with oxo (—C═O), —NH—, —N—C₁₋₃ alkyl-, or —O—.

In some embodiment, Ring B_{2B} is a monocyclic ring selected from 3-7 membered saturated or partially unsaturated carbocyclic ring, a 4-7 membered saturated or partially unsaturated monocyclic heterocyclic ring having 1-4 heteroatoms independently selected from nitrogen, oxygen and sulfur, a benzene (i.e., phenyl) ring, or 5-6 membered heteroaryl ring having 1-4 heteroatoms independently selected from nitrogen, oxygen and sulfur.

In some embodiment, 0-3 methylene moieties (—CH₂—) contained in the Ring B_{2B} may be replaced with oxo (—C═O), —NH—, —N—C₁₋₃ alkyl-, or —O—.

In some embodiment, Ring B_{2A} and Ring B_{2B} taken together form a fused-heterocyclic with two shared/border atoms between Ring B_{2A} and Ring B_{2B}, including E_{2A} and E_{2B}.

In some embodiment, each shared/border atoms can be carbon or heteroatom independently selected from nitrogen, oxygen, and sulfur.

In some embodiment, each R^{B2A} and R^{B2B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R′, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B2A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{B2B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R

In some embodiment, each R is independently hydrogen or C₁-C₆ alkyl.

In some embodiment, each subscript q3 and q4 is independently 0, 1, 2, 3, 4, 5, 6, 7, 8, 9, or 10.

In some embodiment, the compound of the present disclosure is selected from the following [Table B-2].

TABLE B-2

| Example Number | Structure |
|---|---|
| 1 | 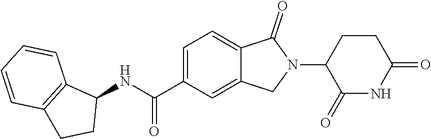 [SMILES: O=C1CCC(N2Cc3cc(C(=O)N[C@H]4CCc5ccccc54)ccc3C2=O)C(=O)N1] |
| 2 | 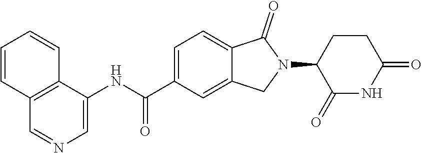 [SMILES: O=C1CC[C@H](N2Cc3cc(C(=O)Nc4cncc5ccccc45)ccc3C2=O)C(=O)N1] |
| 3 | 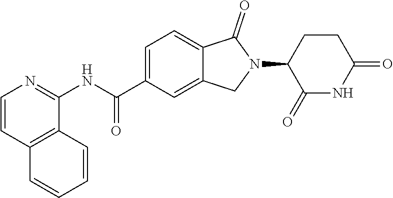 [SMILES: O=C1CC[C@H](N2Cc3cc(C(=O)Nc4nccc5ccccc45)ccc3C2=O)C(=O)N1] |
| 4 | 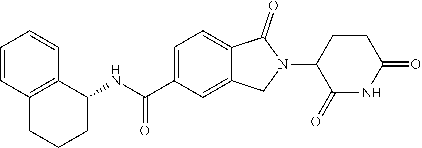 [SMILES: O=C1CCC(N2Cc3cc(C(=O)N[C@@H]4CCCc5ccccc54)ccc3C2=O)C(=O)N1] |
| 5 | 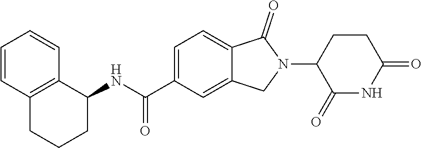 [SMILES: O=C1CCC(N2Cc3cc(C(=O)N[C@H]4CCCc5ccccc54)ccc3C2=O)C(=O)N1] |
| 6 | 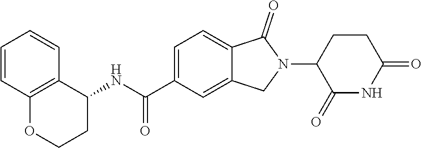 [SMILES: O=C1CCC(N2Cc3cc(C(=O)N[C@@H]4CCOc5ccccc54)ccc3C2=O)C(=O)N1] |
| 7 | 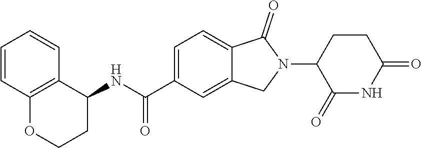 [SMILES: O=C1CCC(N2Cc3cc(C(=O)N[C@H]4CCOc5ccccc54)ccc3C2=O)C(=O)N1] |
| 8 | 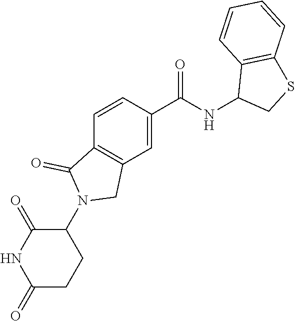 [SMILES: O=C1CCC(N2Cc3cc(C(=O)NC4CSc5ccccc54)ccc3C2=O)C(=O)N1] |
| 9 | 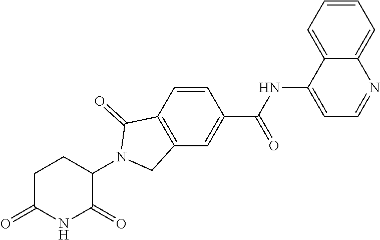 [SMILES: O=C1CCC(N2Cc3cc(C(=O)Nc4ccnc5ccccc45)ccc3C2=O)C(=O)N1] |
| 59 | 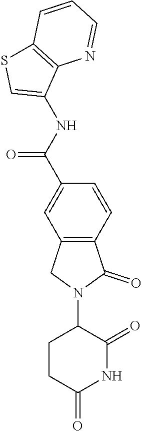 [SMILES: O=C1CCC(N2Cc3cc(C(=O)Nc4csc5cccnc45)ccc3C2=O)C(=O)N1] |
| 60 | 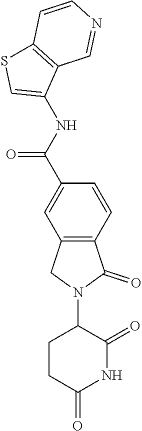 [SMILES: O=C1CCC(N2Cc3cc(C(=O)Nc4csc5ccncc45)ccc3C2=O)C(=O)N1] |
| 61 | 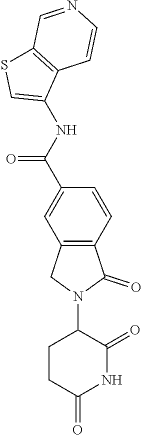 [SMILES: O=C1CCC(N2Cc3cc(C(=O)Nc4csc5cnccc45)ccc3C2=O)C(=O)N1] |
| 62 | 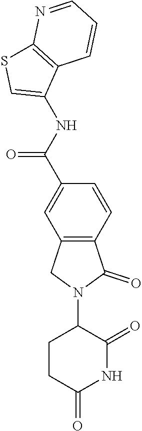 [SMILES: O=C1CCC(N2Cc3cc(C(=O)Nc4csc5ncccc45)ccc3C2=O)C(=O)N1] |
| 67 | 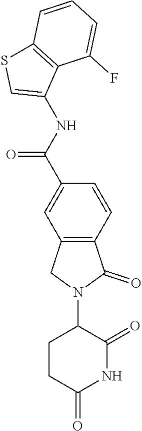 [SMILES: O=C1CCC(N2Cc3cc(C(=O)Nc4csc5cccc(F)c45)ccc3C2=O)C(=O)N1] |
| 68 | 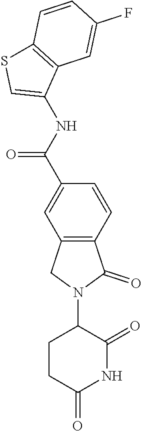 [SMILES: O=C1CCC(N2Cc3cc(C(=O)Nc4csc5ccc(F)cc45)ccc3C2=O)C(=O)N1] |
| 69 | 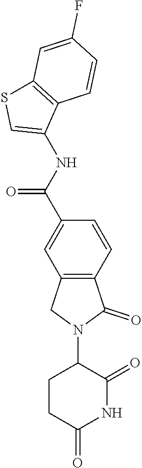 [SMILES: O=C1CCC(N2Cc3cc(C(=O)Nc4csc5cc(F)ccc45)ccc3C2=O)C(=O)N1] |
| 70 | 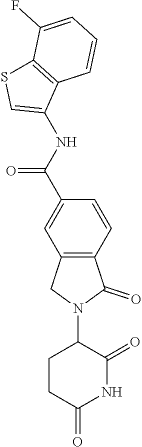 [SMILES: O=C1CCC(N2Cc3cc(C(=O)Nc4csc5c(F)cccc45)ccc3C2=O)C(=O)N1] |
| 89 | 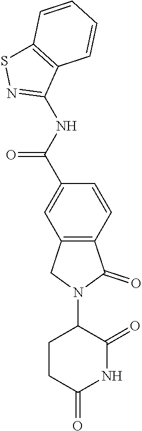 [SMILES: O=C1CCC(N2Cc3cc(C(=O)Nc4nsc5ccccc45)ccc3C2=O)C(=O)N1] |
| 91 | 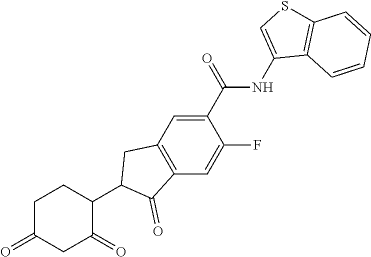 [SMILES: O=C1CCC(C2Cc3cc(C(=O)Nc4csc5ccccc45)c(F)cc3C2=O)C(=O)C1] |
| 95 | 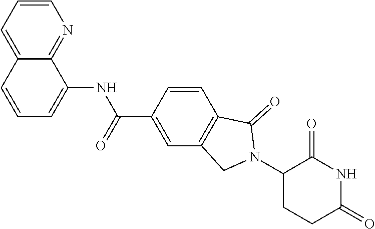 [SMILES: O=C1CCC(N2Cc3cc(C(=O)Nc4cccc5cccnc45)ccc3C2=O)C(=O)N1] |

In some embodiment, the compound has the structure of Formula (II-c-1):

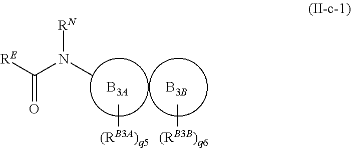

In some embodiment, each Ring B_{3A} and Ring B_{3B} is monocyclic ring selected from 4-7 membered saturated or partially unsaturated ring having 0-4 heteroatoms independently selected from nitrogen, oxygen, and sulfur.

In some embodiment, 0-4 methylene moieties (—CH₂—) contained in the Ring B_{3A} or Ring B_{3B} may be independently replaced with oxo (—C═O), —NH—, —N—C₁₋₃ alkyl-, or —O—.

In some embodiment, Ring B_{3A} and Ring B_{3B} taken together form a spirocyclic or spiro-heterocyclic ring with one shared/border carbon atom between Ring B_{3A} and Ring B_{3B}.

In some embodiment, each R^{B3A} and R^{B3B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B3A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, two of R^{B3B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, each subscript q5 and q6 is independently 0, 1, 2, 3, 4, 5, 6, 7, 8, 9, or 10.

In some embodiment, the compound comprises additional substituent of an optionally substituted ring of Ring B_{3C} as depicted in the Formula (II-c-2) below:

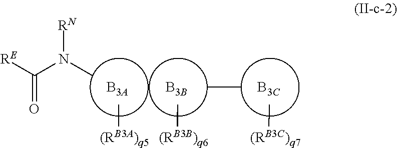

In some embodiment, Ring B_{3C} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur.

In some embodiment, 0-3 methylene moieties (—CH₂—) contained in the Ring B_{3C} may be independently replaced with oxo (—C═O), —NH—, —N—C₁₋₃ alkyl-, or —O—.

In some embodiment, R^{B3C} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R′)(R^{b}), -alkyl-OC(O)N(R^{a})(R^{b}), -alkyl-N(R^{a})C(O)R^{a}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B3C} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₅-C₁₃ spiroalkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript q7 is 0, 1, 2, 3, 4, 5, 6, 7, 8, 9, or 10.

In some embodiment, the compound has the structure of Formula (II-c-3) or (I-c-4):

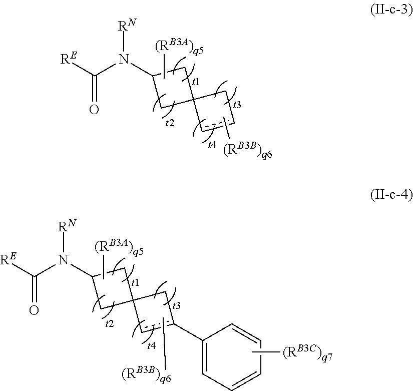

In some embodiment, each subscript t1, t2, t3 and t4 is independently 1 or 2.

In some embodiment, spirocyclic or spiro-heterocyclic ring depicted in Formula (II-c-3) or (II-c-4) above, optionally comprises one double bond therein depicted as dotted line, and 0-2 methylene moieties (—CH₂—) contained in the ring may be replaced with oxo (—C═O), —NH—, —N—C₁₋₃ alkyl-, —O—.

In some embodiment, each R^{B3A}, R^{B3B} and R^{B3C} is independently hydrogen, deuterium, halogen, C₁-C₄ alkyl (i.e., straight or branched), C₁-C₃ alkoxy, C₁-C₃ haloalkyl, C₁-C₃ haloalkoxy, or cyano.

In some embodiment, the compound of the present disclosure is selected from the following [Table B-3].

TABLE B-3

| Example Number | Structure |
|---|---|
| 100 | 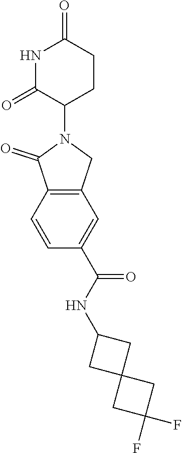 [SMILES: O=C1CCC(N2Cc3cc(C(=O)NC4CC5(C4)CC(F)(F)C5)ccc3C2=O)C(=O)N1] |
| 213 | 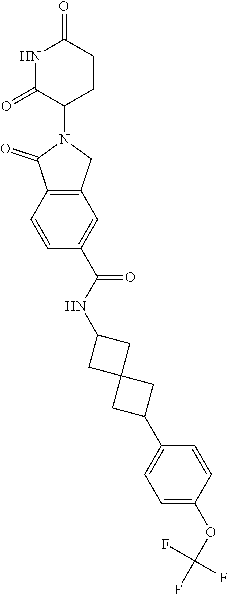 [SMILES: O=C1CCC(N2Cc3cc(C(=O)NC4CC5(C4)CC(c4ccc(OC(F)(F)F)cc4)C5)ccc3C2=O)C(=O)N1] |
| 214 | 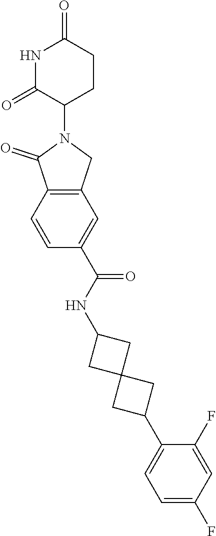 [SMILES: O=C1CCC(N2Cc3cc(C(=O)NC4CC5(C4)CC(c4ccc(F)cc4F)C5)ccc3C2=O)C(=O)N1] |
| 215 | 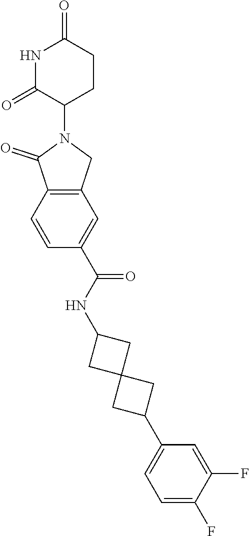 [SMILES: O=C1CCC(N2Cc3cc(C(=O)NC4CC5(C4)CC(c4ccc(F)c(F)c4)C5)ccc3C2=O)C(=O)N1] |
| 216 | 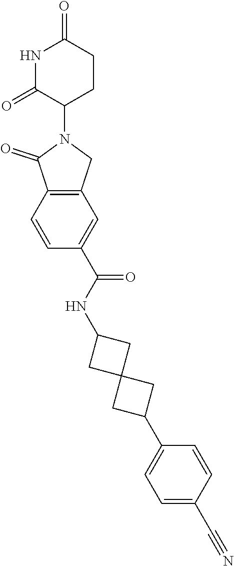 [SMILES: N#Cc1ccc(C2CC3(CC(NC(=O)c4ccc5c(c4)CN(C4CCC(=O)NC4=O)C5=O)C3)C2)cc1] |
| 217 | 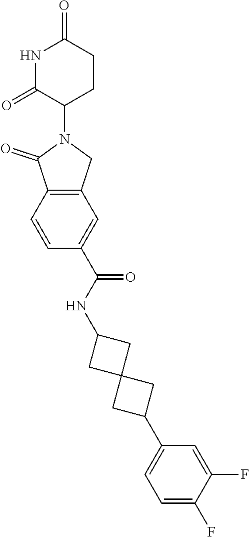 [SMILES: O=C1CCC(N2Cc3cc(C(=O)NC4CC5(C4)CC(c4ccc(F)c(F)c4)C5)ccc3C2=O)C(=O)N1] |
| 218 | 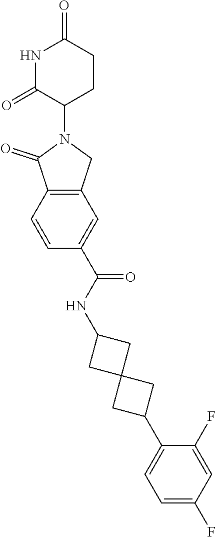 [SMILES: O=C1CCC(N2Cc3cc(C(=O)NC4CC5(C4)CC(c4ccc(F)cc4F)C5)ccc3C2=O)C(=O)N1] |
| 219 | 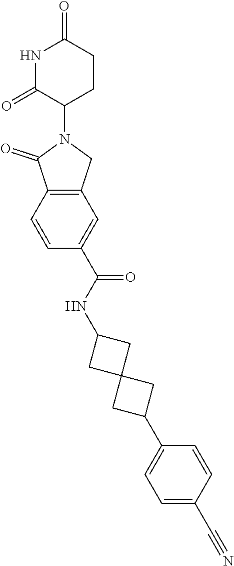 [SMILES: N#Cc1ccc(C2CC3(CC(NC(=O)c4ccc5c(c4)CN(C4CCC(=O)NC4=O)C5=O)C3)C2)cc1] |
| 225 | 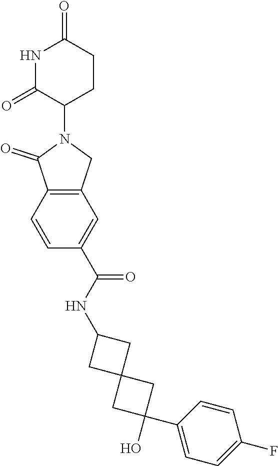 [SMILES: O=C1CCC(N2Cc3cc(C(=O)NC4CC5(C4)CC(O)(c4ccc(F)cc4)C5)ccc3C2=O)C(=O)N1] |
| 226 | 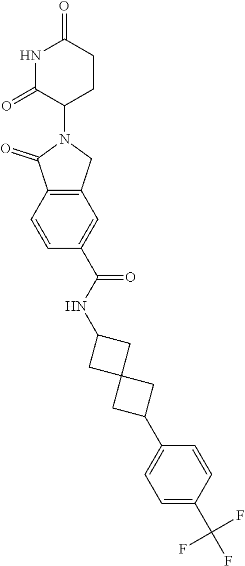 [SMILES: O=C1CCC(N2Cc3cc(C(=O)NC4CC5(C4)CC(c4ccc(C(F)(F)F)cc4)C5)ccc3C2=O)C(=O)N1] |
| 227 | 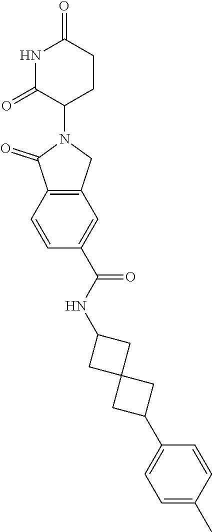 [SMILES: Cc1ccc(C2CC3(CC(NC(=O)c4ccc5c(c4)CN(C4CCC(=O)NC4=O)C5=O)C3)C2)cc1] |
| 253 | 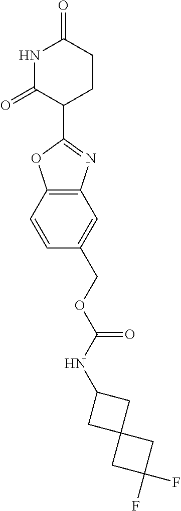 [SMILES: O=C1CCC(c2nc3cc(COC(=O)NC4CC5(C4)CC(F)(F)C5)ccc3o2)C(=O)N1] |
| 254 | 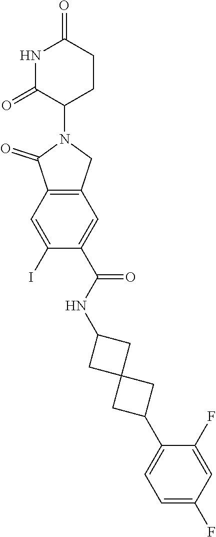 [SMILES: O=C1CCC(N2Cc3cc(C(=O)NC4CC5(C4)CC(c4ccc(F)cc4F)C5)c(I)cc3C2=O)C(=O)N1] |
| 255 | 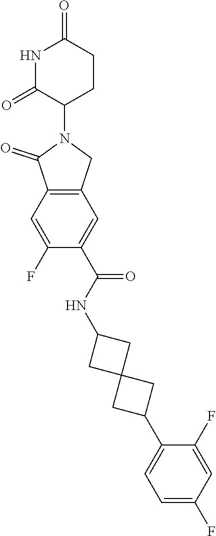 [SMILES: O=C1CCC(N2Cc3cc(C(=O)NC4CC5(C4)CC(c4ccc(F)cc4F)C5)c(F)cc3C2=O)C(=O)N1] |
| 256 | 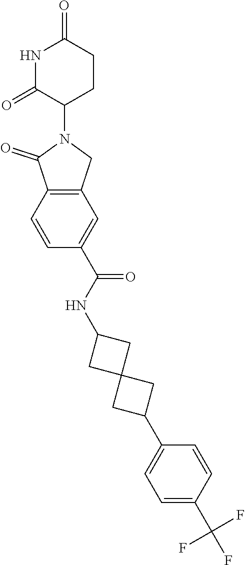 [SMILES: O=C1CCC(N2Cc3cc(C(=O)NC4CC5(C4)CC(c4ccc(C(F)(F)F)cc4)C5)ccc3C2=O)C(=O)N1] |
| 257 | 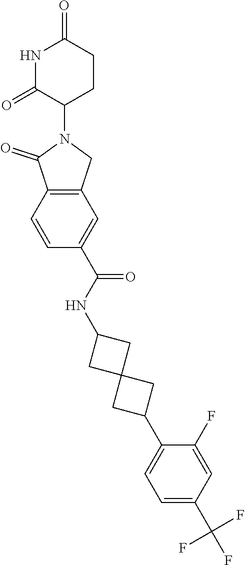 [SMILES: O=C1CCC(N2Cc3cc(C(=O)NC4CC5(C4)CC(c4ccc(C(F)(F)F)cc4F)C5)ccc3C2=O)C(=O)N1] |
| 258 | 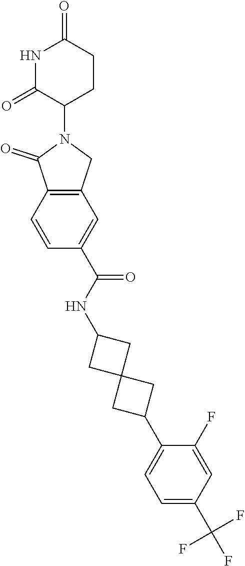 [SMILES: O=C1CCC(N2Cc3cc(C(=O)NC4CC5(C4)CC(c4ccc(C(F)(F)F)cc4F)C5)ccc3C2=O)C(=O)N1] |
| 260 | 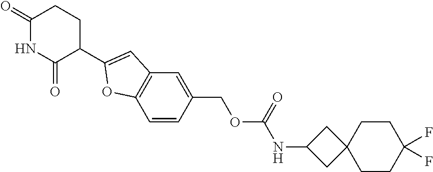 [SMILES: O=C1CCC(c2cc3cc(COC(=O)NC4CC5(CCC(F)(F)CC5)C4)ccc3o2)C(=O)N1] |

In some embodiment, the compound has the structure of Formula (II-d):

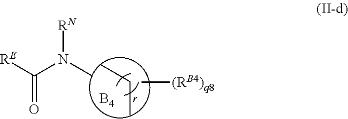

In some embodiment, Ring B₄ is fully saturated 5-10 membered bridged-cyclic ring or bridged-heterocyclic ring having 0-3 heteroatoms independently selected from nitrogen, oxygen, and sulfur.

In some embodiment, 0-3 methylene moieties (—CH₂—) contained in the Ring B₄ may be independently replaced with oxo (—C═O), —NH—, —N—C₁₋₃ alkyl-, or —O—.

In some embodiment, a subscript r is 1, 2 or 3.

In some embodiment, R^{B4} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R′)(R^{b}), -alkyl-OC(O)N(R^{a})(R^{b}), -alkyl-N(R^{a})C(O)R^{a}, —S(O)(═N(R^{b}))R^{c}, —N═S(O)R^{b}R^{c}, ═NR^{b}, SO₂N(R^{b})R^{c}, or N(R^{b})SO₂R^{c}.

In some embodiment, two of R^{B4} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d}.

In some embodiment, each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R.

In some embodiment, each R is independently hydrogen or C₁-C₆ alkyl.

In some embodiment, a subscript q8 is 0, 1, 2, 3, 4, 5, 6, 7, 8, 9, or 10.

In some embodiment, the compound has the structure of Formula (II-d-1) or (II-d 2):

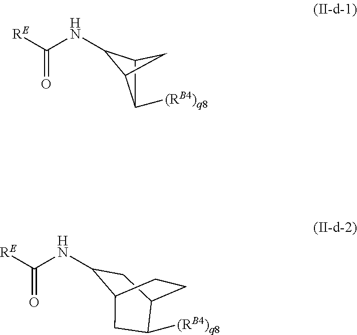

In some embodiment, each R^{B4} is independently hydrogen, deuterium, halogen.

In some embodiment, the compound of the present disclosure is selected from the following [Table B-4].

TABLE B-4

| Example Number | Structure |
|---|---|
| 71 | 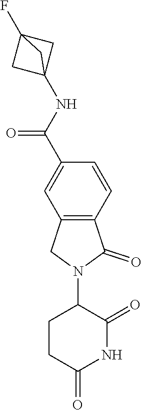 [SMILES: O=C1CCC(N2Cc3cc(C(=O)NC45CC(F)(C4)C5)ccc3C2=O)C(=O)N1] |
| 72 | 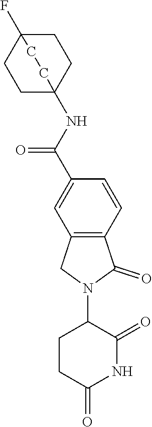 [SMILES: O=C1CCC(N2Cc3cc(C(=O)NC45CCC(F)(CC4)CC5)ccc3C2=O)C(=O)N1] |

As used herein, the term “compound” unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition, it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns. Likewise, it is understood that, in any compound described, all tautomeric and conformeric forms are also intended to be included. A conformer is a structure that is a conformational isomer. Conformational isomerism is the phenomenon of molecules with the same structural formula but different conformations (conformers) of atoms about a rotating bond.

As used herein, the term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, any “R” group(s) represent substituents that can be attached to the indicated atom. An R group may be substituted or unsubstituted. Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents. If no substituents are indicated, it is meant that the indicated “optionally substituted” or “substituted” group may be individually and independently substituted with one or more group(s) individually and independently selected from alkyl, cycloalkyl, aryl, heteroaryl, heterocyclyl, aralkyl, heteroaralkyl, heterocyclyl(alkyl), hydroxy, alkoxy, cycloalkoxy, aryloxy, acyl, mercapto, alkylthio, arylthio, cyano, halogen, C-amido, N-amido, C-carboxy, O-carboxy, isocyanato, thiocyanato, isothiocyanato, nitro, haloalkyl, haloalkoxy, amino (including mono-substituted amino and di-substituted amino), and alkylamino. When a group is not described as “optionally substituted,” “unsubstituted” or “substituted,” such group is unsubstituted unless the definition of such group states otherwise.

As used herein, “Cₐ to C_{b}” or “C_{a-b}” in which “a” and “b” are integers refer to the number of carbon atoms in an alkyl, alkenyl or alkynyl group, or the number of carbon atoms in the ring of a cycloalkyl, aryl, heteroaryl, heterocycloalkyl or heterocyclyl group. That is, the alkyl, alkenyl, alkynyl, ring of the cycloalkyl, ring of the aryl, ring of the heterocycloalkyl, ring of the heteroaryl or ring of the heterocyclyl can contain from “a” to “b”, inclusive, carbon atoms.

As used herein, the term “unsaturated” or “partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups). Likewise, “saturated” refers to a group that does not contain a double or triple bond, i.e., contains all single bonds.

As used herein, the term “Optional” or “optionally” means that a subsequently described event or circumstance may or may not occur and that the description includes instances when the event or circumstance occurs and instances in which it does not. For example, “optionally substituted alkyl” means that the alkyl radical may or may not be substituted and that the description includes both substituted alkyl radicals and alkyl radicals having no substitution.

As used herein, the term “optionally substituted” or “substituted” means that the referenced group is optionally substituted with one or more additional group(s).

As used herein, the term “aliphatic” includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the disclosure contain 1-20 aliphatic carbon atoms. In certain other embodiments, the alkyl, alkenyl, and alkynyl groups employed in the disclosure contain 1-10 aliphatic carbon atoms. In yet other embodiments, the alkyl, alkenyl, and alkynyl groups employed in the disclosure contain 1-8 aliphatic carbon atoms. In still other embodiments, the alkyl, alkenyl, and alkynyl groups employed in the disclosure contain 1-6 aliphatic carbon atoms. In yet other embodiments, the alkyl, alkenyl, and alkynyl groups employed in the disclosure contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

As used herein, the term “alkyl” refers to a radical of a straight-chain or branched saturated hydrocarbon group having from 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

As used herein, the term “haloalkyl” refers to an alkyl group in which one or more of the hydrogen atoms has been replaced by one or more halogen atom(s). The term “Cₙ₋ₘ haloalkyl” or “Cₙ-Cₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

As used herein, the term “aminoalkyl” refers to an alkyl group as defined above, wherein one of the alkyl group's hydrogen atoms has been replaced with —NH₂, —NH(C₁-C₆ alkyl), or —N(C₁-C₆ alkyl)₂. In one embodiment, an aminoalkyl group has from 1 to 6 carbon atoms. Non-limiting examples of aminoalkyl groups include —CH₂NH₂, —CH₂N(CH₃)₂, —CH₂NH₂, and —CH₂NH(CH)₃. The term “C₁-C₆ aminoalkyl” refers to an aminoalkyl group having from 1 to 6 carbon atoms.

As used herein, the term “alkoxy” refers to a group containing an alkyl radical attached through an oxygen linking atom, wherein alkyl is as defined above. An alkoxy group can have a specified number of carbon atoms. For example, the terms “(C₁-C₆)alkoxy” and “(C₁₋₆)alkoxy” refer to an alkyl radical, having at least 1 and up to 6 carbon atoms attached through an oxygen linking atom. Likewise, the terms “(C₁-C₄)alkoxy” and “(C₁₋₄)alkoxy” refer to an alkyl radical having at least 1 and up to 4 carbon atoms attached through an oxygen linking atom. Exemplary alkoxy groups include, but are not limited to, methoxy, ethoxy, n-propoxy, isopropoxy, n-butoxy, s-butoxy, and t-butoxy.

As used herein, the term “haloalkoxy” refers to an alkoxy group in which the alkyl moiety is substituted with one or more halogens, wherein halogen is independently selected from fluoro, chloro, bromo, and iodo. A haloalkoxy group can have a specified number of carbon atoms. For example, the term “(C₁-C₆)haloalkoxy refers to a haloalkyl radical, having at 1 to 6 carbon atoms attached through an oxygen linking atom. Representative haloalkoxy groups include, but are not limited to difluoromethoxy (—OCHCF₂), trifluoromethoxy (—OCF₃), tetrafluoroethoxy (—OCF₂CHF₂) and the like.

As used herein, the term “alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃) may be an (E)- or (Z)-double bond.

As used herein, the term “alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

As used herein, the term “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The term “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, or silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered, non-aromatic ring system having ring carbon atoms and 1-4 ring heteroatoms, wherein each heteroatom is independently selected from nitrogen, oxygen, sulfur, boron, phosphorus, or silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, or sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, or sulfur (“5-6 membered heterocyclyl”). In some embodiments, the 5-6 membered heterocyclyl has 1-3 ring heteroatoms selected from nitrogen, oxygen, or sulfur. In some embodiments, the 5-6 membered heterocyclyl has 1-2 ring heteroatoms selected from nitrogen, oxygen, or sulfur. In some embodiments, the 5-6 membered heterocyclyl has one ring heteroatom selected from nitrogen, oxygen, or sulfur.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

As used herein, the term “cycloalkyl” refers to a monovalent chain of carbon atoms, a portion of which forms a ring. It is understood that in embodiments that include cycloalkyl, illustrative variations of those embodiments include lower cycloalkyl, such as C₃-C₈, C₃-C₇, C₃-C₆, C₃-C₅, C₄-C₈, C₄-C₇, C₄-C₆ cycloalkyl, cyclopropyl, cyclohexyl, 3-ethylcyclopentyl, and the like.

As used herein, the term “bicycloalkyl” refers to two cycloalkyl groups, defined hereinabove, connecting with each other to form a bridged, fused or spiro bicyclic compound.

As used herein, the term “fused bicycloalkyl” refers to two cycloalkyl groups which share two adjacent atoms. In other words, the rings share one covalent bond, i.e. the so-called bridgehead atoms are directly connected.

As used herein, the term “bridged bicycloalkyl” refers to two cycloalkyl groups of which moieties have more than two atoms in common. That is, two rings do not share adjacent carbon atoms, but share three or more atoms, separating the two bridgehead atoms by a bridge containing at least one atom. Examples include, but are not limited to, bicyclo[3.2.1]heptyl (“norbornyl”), bicyclo[2.2.2]octyl, and the like.

As used herein, the term “spirobicycloalkyl” refers to bicyclic groups in which the two rings are attached at a single carbon atom that is a member of each of the two rings. The term includes both spirobicycloalkyls, in which the two rings are cycloalkyl rings attached at a single carbon atom that is a member of each of the two rings, and spirobicycloheteroalkyls, in which one ring is a heterocyclyl ring and the other ring is a cycloalkyl ring attached at a single carbon atom that is a member of each of the two rings, or in which both rings are heterocyclyl rings attached at a single carbon atom that is a member of each of the two rings. Examples of spirobicyclyl groups include spiro[3.3]heptenyl, spiro[3.4]octanyl, azaspiro[3.3]heptanyl, oxaazaspiro[3.3]heptanyl, oxa-azaspiro[3.3]heptanyl, and azaspiro[3.4]octanyl.

As used herein, “heterocycloalkyl” refers to a non-aromatic ring, which has at least one heteroatom ring member independently selected from nitrogen, sulfur, and oxygen. Heterocycloalkyl groups can include mono- or polycyclic (e.g., fused, bridged, or spiro) ring systems. Heterocycloalkyl also includes one or more aromatic rings fused to the non-aromatic heterocycloalkyl ring. The ring-forming carbon atoms of a heterocycloalkyl group can be optionally substituted by oxo. The ring-forming heteroatoms of the heterocycloalkyl group can be oxidized to form an N-oxide or a sulfonyl group. It is understood that in embodiments that include heterocycloalkyl, illustrative variations of those embodiments include lower cycloalkyl, such as C₃-C₈, C₃-C₇, C₃-C₆, C₃-C₅, C₄-C₈, C₄-C₇, C₄-C₆ heterocycloalkyl and the like. Examples of heterocycloalkyl group include morpholine ring, pyrrolidine ring, piperazine ring, piperidine ring, tetrahydropyran ring, tetrahyropyridine, azetidine ring, tetrahydrofuran, etc.

As used herein, “heterobicycloalkyl” refers to a bicycloalkyl structure, which is unsubstituted or substituted, in which at least one carbon atom is replaced with a heteroatom independently selected from oxygen, nitrogen, and sulfur.

As used herein, the term “aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups, wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

As used herein, the term “aralkyl” refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

As used herein, the term “heteroaryl” refers to a radical of a 5-10 membered, monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen or sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, or sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, or sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, or sulfur (“5-6 membered heteroaryl”). In some embodiments, the 5-6 membered heteroaryl has 1-3 ring heteroatoms selected from nitrogen, oxygen, or sulfur. In some embodiments, the 5-6 membered heteroaryl has 1-2 ring heteroatoms selected from nitrogen, oxygen, or sulfur. In some embodiments, the 5-6 membered heteroaryl has 1 ring heteroatom selected from nitrogen, oxygen, or sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “heteroaralkyl” is a subset of alkyl and heteroaryl and refers to an optionally substituted alkyl group substituted by an optionally substituted heteroaryl group.

In some embodiment, the compound of the present disclosure is for use in degrading GSPT1 (G1 To S Phase Transition 1) protein and/or reducing level thereof.

In some embodiment, the compound of the present disclosure is for use in inhibiting GSPT1 activity.

In some embodiment, the present disclosure provides a pharmaceutical composition comprising: the compound of the present disclosure (represented by Formula (I) or (II)), or the pharmaceutically acceptable salt, enantiomer, stereoisomer or prodrug thereof, and optionally a pharmaceutically acceptable excipient or carrier.

As used herein, the term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to a carrier that is useful in preparing a pharmaceutical composition that is generally safe, non-toxic and neither biologically nor otherwise-undesirable, and is acceptable for veterinary use as well as human pharmaceutical use. A “pharmaceutically acceptable carrier” as used in the specification and claims can include both one and more than one such carrier. By “pharmaceutically acceptable” it is meant the carrier must be compatible with the other ingredients of the formulation and not deleterious to the recipient thereof.

As used herein, the term “pharmaceutically acceptable salts” refers to salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The compounds represented by Formula (I) or (II), or a pharmaceutically acceptable salt, enantiomer, stereoisomer or prodrug thereof degrade GSPT1 (G1 To S Phase Transition 1) protein.

In some embodiment, the pharmaceutical composition is for use in degrading GSPT1 protein and/or reducing level thereof.

In some embodiment, the pharmaceutical composition is for use in inhibiting GSPT1 activity.

The present disclosure also provides a method of degrading GSPT1 protein and/or reducing level thereof in a subject in need thereof, the method comprising administering to the subject an effective amount of the pharmaceutical composition of the present disclosure.

The present disclosure also provides a method of inhibiting GSPT1 activity in a subject in need thereof, the method comprising administering to the subject an effective amount of the pharmaceutical composition of the present disclosure.

The present disclosure also provides a method of preventing or treating GSPT1-related disease in a subject in need thereof, the method comprising administering to the subject an effective amount of the pharmaceutical composition of the present disclosure.

In some embodiments, the compounds of the present disclosure may bind to E3 ligase, for example, cereblon (CRBN), altering the specificity of the complex to induce the ubiquitination and degradation of GSPT1.

In some embodiments, the present disclosure provides compounds and compositions comprising an E3 ligase binding moiety that can bind to an E3 ligase (e.g., CRBN) and target protein binding moiety that can bind to a target protein, which results in the ubiquitination of a target protein and leads to degradation of the target protein by the proteasome.

As used herein, the term “degrade” or “degradation” refers to the degradation of a target protein mediated by an E3 ligase, for example, CRBN, resulting in a reduction of the protein levels. In one embodiment, the target protein can be GSPT1 protein. In certain embodiments, the target protein preferably can be GSPT1.

As used herein, the term “GSPT1” refers to G1 to S phase transition protein 1 homolog and is involved in translation termination in response to the termination codons UAA, UAG, and UGA, and is also involved in regulation of mammalian cell growth. GSPT1 stimulates the activity of eRF1 and is a component of the transient SURF complex, which recruits UPF1 to stalled ribosomes in the context of nonsense-mediated decay (NMD) of mRNAs.

As used herein, the term “effective amount” refers to that amount of an active agent being administered sufficient to degrade GSPT1 and/or inhibit GSPT1 activity. In certain embodiments, the term “effective amount” refers to that amount of an active agent being administered sufficient to treat a GSPT1-related disease, preferably GSPT1-mediated cancer.

As used herein, the term “GSPT1-related disease”, “GSPT1-associated disease” and/or “GSTP1-mediated disease” is used to refer to a disease state or condition that result from hyper- or hypo-activation, dysfunction or malfunction of GSPT1 protein in various signaling pathways thereof.

In some embodiments, the “GSPT1-related disease”, “GSPT1-associated disease” and/or “GSTP1-mediated disease” is cancer, proliferative disorder, inflammation, autoimmune disease, viral infections, or immunological disease.

The cancer may include, but are not limited to, gastric adenocarcinoma, colorectal cancer, li-fraumeni syndrome, esophageal cancer, hepatocellular carcinoma, adenocarcinoma, small intestine cancer, pancreatic cancer, lung cancer, brain cancer, ovarian cancer, cervical cancer, prostate cancer, colon cancer, head and neck cancer, stomach cancer, hepatobiliary cancer, rectal cancer, kidney cancer, bladder cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, liver cancer, skin cancer, urothelial carcinoma, renal cancer, renal cell carcinoma, oropharyngeal cancer, carcinoma, melanoma, and/or breast cancer.

The proliferative disorder may include, but are not limited to, multiple myeloma, acute myeloid leukemia, T-cell acute myeloid leukemia, B-cell acute myeloid leukemia, diffuse large B-cell lymphoma (DLBCL), active B-cell diffuse large B-cell lymphoma (ABC DLBCL), myelodysplastic syndrome, Myeloproliferative neoplasms, Myelodysplastic-myeloproliferative neoplasms, malignant lymphomas, head and neck tumors, brain tumors, brain metastases, non-small cell tumors, small cell lung tumors, gastrointestinal tumors, endocrine tumors, mammary tumors, gynecological tumors, urological tumors, renal tumors, bladder tumors, prostate tumors, skin tumors, and sarcomas, cervical tumor, lung tumor, colon tumor, colorectal carcinoma, lymphoma psoriasis, keloids, benign prostate hyperplasia (BPH), and/or metastases thereof.

In addition, the proliferative disorder may include diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses, haematological tumors, solid tumors, and/or metastases thereof, e.g. leukaemias and myelodysplastic syndrome, malignant lymphomas, head and neck tumors including brain tumors and brain metastases, tumors of the thorax including non-small cell and small cell lung tumors, gastrointestinal tumors, endocrine tumors, mammary and other gynaecological tumors, urological tumors including renal, bladder and prostate tumors, skin tumors, and sarcomas, cervical tumor, a lung tumor (such as lung carcinoma), a colon tumor (such as colorectal carcinoma), a lymphoma (such as diffuse large B-cell lymphoma) and/or metastases thereof.

In another embodiment, the proliferative disorder may also include, but are not limited to, hyper-proliferative disorder or a disorder responsive to induction of cell death e.g. apoptosis. Hyper-proliferative disorders include but are not limited, e.g., psoriasis, keloids, and other hyperplasias affecting the skin, benign prostate hyperplasia (BPH), solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to small-cell and non-small-cell lung carcinoma, as well as bronchial adenoma and pleuropulmonary blastoma. Examples of brain cancers include, but are not limited to brain stem and hypothalamic glioma, cerebellar and cerebral astrocytoma, medulloblastoma, ependymoma, as well as neuroectodermal and pineal tumor. Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, oesophageal, gallbladder, gastric, pancreatic, rectal, small-intestine, and salivary gland cancers. Tumors of the urinary tract include, but are not limited to bladder, penile, kidney, renal pelvis, ureter, urethral and human papillary renal cancers. Eye cancers include, but are not limited to intraocular melanoma and retinoblastoma. Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system. Sarcomas include, but are not limited to sarcoma of the soft tissue, osteosarcoma, malignant fibrous histiocytoma, lymphosarcoma, and rhabdomyosarcoma. Leukemias include, but are not limited to acute myeloid leukemia (AML), acute lymphoblastic leukemia (ALL), chronic lymphocytic leukemia (CLL), chronic myelogenous leukemia (CML), and hairy cell leukemia.

Furthermore, the proliferative disorder may also include, but are not limited to, benign and malignant neoplasia, more specifically haematological tumors, solid tumors, and/or metastases thereof, e.g. leukaemias and myelodysplastic syndrome, malignant lymphomas, head and neck tumors including brain tumors and brain metastases, tumors of the thorax including non-small cell and small cell lung tumors, gastrointestinal tumors, endocrine tumors, mammary and other gynaecological tumors, urological tumors including renal, bladder and prostate tumors, skin tumors, and sarcomas, and/or metastases thereof, especially haematological tumors, solid tumors, and/or metastases of breast, bladder, bone, brain, central and peripheral nervous system, cervix, colon, endocrine glands (e.g. thyroid and adrenal cortex), endocrine tumors, endometrium, esophagus, gastrointestinal tumors, germ cells, kidney, liver, lung, larynx and hypopharynx, mesothelioma, ovary, pancreas, prostate, rectum, renal, small intestine, soft tissue, stomach, skin, testis, ureter, vagina and vulva as well as malignant neoplasias including primary tumors in said organs and corresponding secondary tumors in distant organs (tumor metastases). Haematological tumors can e.g be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

In some embodiments, the (GSPT1-mediated) cancer is selected from the group consisting of gastric adenocarcinoma, colorectal cancer, li-fraumeni syndrome, esophageal cancer, hepatocellular carcinoma, adenocarcinoma, small intestine cancer, pancreatic cancer, lung cancer, brain cancer, ovarian cancer, cervical cancer, prostate cancer, colon cancer, head and neck cancer, stomach cancer, hepatobiliary cancer, rectal cancer, kidney cancer, bladder cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, liver cancer, skin cancer, urothelial carcinoma, renal cancer, renal cell carcinoma, oropharyngeal cancer, carcinoma, melanoma and breast cancer.

In some embodiments, the (GSPT1-mediated) cancer is selected from the group consisting of breast cancer, colorectal cancer, lung cancer, e.g. SCLC, NSCLC, liver cancer, neuroendocrine cancer, e.g., neuroendocrine prostate cancer (for example, NEPC (castration-resistant neuroendocrine prostate cancer)) and lung neuroendocrine tumors (Lu-NETs), stomach cancer, pancreatic cancer, gastric cancer, esophageal cancer, bladder cancer, skin cancer, and head and neck cancer.

As used herein, the term “treat,” “treatment,” or “treating,” refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, the term “preventing” refers to a slowing of the disease or of the onset of the disease or the symptoms thereof. Preventing a disease or disorder can include stopping the onset of the disease or symptoms thereof.

The pharmaceutical compositions described herein can be administered to a human patient per se, or in pharmaceutical compositions where they are mixed with other active ingredients, as in combination therapy, or excipients, or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound described herein, including a compound of Formula (I) or a pharmaceutically acceptable salt, enantiomer, stereoisomer or prodrug thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical excipient may also be prepared, placed in an appropriate container, and labeled for treatment of an indicated condition.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example, syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively, dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between about 0.01 mg and 3,000 mg of each active ingredient, preferably between about 1 mg and 700 mg, e.g., about 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or Id₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for about 10 to 90% of the time, preferably between about 30 to 90% and most preferably between about 50 to 90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

### I. Synthesis of Compounds

### Example 1

### Synthesis of N—((S)-2,3-dihydro-1H-inden-1-yl)-2-(2,6-dioxopiperidin-3-yl)-1-oxoisoindoline-5-carboxamide

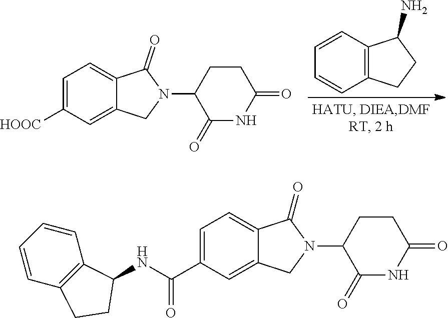

Step 1. Preparation of N—((S)-2,3-dihydro-1H-inden-1-yl)-2-(2,6-dioxopiperidin-3-yl)-1-oxoisoindoline-5-carboxamide. To a stirred solution of 2-(2,6-dioxopiperidin-3-yl)-1-oxo-3H-isoindole-5-carboxylic acid (100 mg, 0.347 mmol, 1 equiv) and (1S)-2,3-dihydro-1H-inden-1-amine (69.3 mg, 0.520 mmol, 1.5 equiv) in DMF (1 ml) were added DIEA (134.5 mg, 1.041 mmol, 3 equiv) and HATU (197.9 mg, 0.520 mmol, 1.5 equiv) in portions at room temperature. The resulting mixture was stirred for 2 h at room temperature. The resulting mixture was diluted with MeCN (1 mL). The residue was purified by reverse-phase flash chromatography to afford N—((S)-2,3-dihydro-1H-inden-1-yl)-2-(2,6-dioxopiperidin-3-yl)-1-oxoisoindoline-5-carboxamide (36 mg, 25%) as an off-white solid.

LC-MS (ES, m/z)=404 [M+1]⁺. ¹H NMR (400 MHz, DMSO-d₆) δ 11.00 (s, 1H), 8.96 (d, J=8.3 Hz, 1H), 8.12 (s, 1H), 8.04 (d, J=8.0 Hz, 1H), 7.81 (d, J=7.9 Hz, 1H), 7.32-7.15 (m, 4H), 5.59 (q, J=8.0 Hz, 1H), 5.14 (dd, J=13.2, 5.1 Hz, 1H), 4.52 (d, J=17.5 Hz, 1H), 4.39 (d, J=17.6 Hz, 1H), 3.06-2.89 (m, 2H), 2.86 (dd, J=16.5, 8.2 Hz, 1H), 2.61 (d, J=18.2 Hz, 2H), 2.49-2.38 (m, 4H), 2.01 (dd, J=12.5, 8.3 Hz, 2H), 1.37 (s, 1H).

### Example 2

### Synthesis of 2-(2,6-dioxopiperidin-3-yl)-N-(isoquinolin-4-yl)-1-oxoisoindoline-5-carboxamide

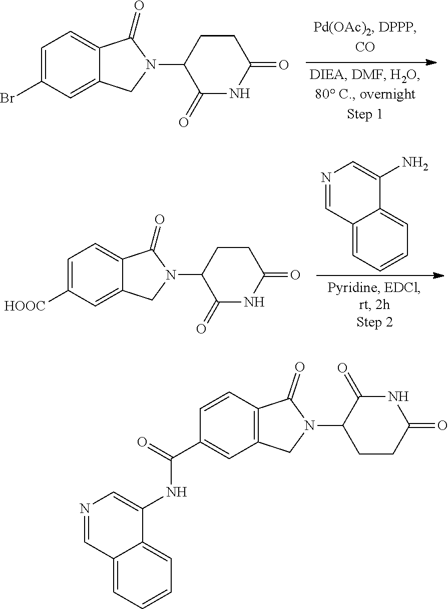 [SMILES: O=C1CCC(N2Cc3cc(Br)ccc3C2=O)C(=O)N1 ; O=C1CCC(N2Cc3cc(C(=O)O)ccc3C2=O)C(=O)N1 ; Nc1cncc2ccccc12 ; O=C1CCC(N2Cc3cc(C(=O)Nc4cncc5ccccc45)ccc3C2=O)C(=O)N1]

Step 1. Preparation of 2-(2,6-dioxopiperidin-3-yl)-1-oxoisoindoline-5-carboxylic acid. To a stirred mixture of 3-(5-bromo-1-oxo-3H-isoindol-2-yl)piperidine-2,6-dione (1 g, 3.095 mmol, 1 equiv) and DIEA (1.20 g, 9.285 mmol, 3 equiv) in DMF (10 ml) and H₂O (1 ml) were added Pd(OAc)₂ (0.07 g, 0.310 mmol, 0.1 equiv) and DPPP (0.26 g, 0.619 mmol, 0.2 equiv) at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. The resulting mixture was stirred for overnight at 80° C. under CO atmosphere. The residue was purified by silica gel column chromatography, eluted with PE/EA (5:1) to afford 2-(2,6-dioxopiperidin-3-yl)-1-oxoisoindoline-5-carboxylic acid (500 mg, 56%) as a brown solid.

Step 2. Preparation of 2-(2,6-dioxopiperidin-3-yl)-N-(isoquinolin-4-yl)-1-oxoisoindoline-5-carboxamide. To a stirred mixture of 2-(2,6-dioxopiperidin-3-yl)-1-oxoisoindoline-5-carboxylic acid (100 mg, 0.347 mmol, 1 equiv) and isoquinolin-4-amine (60.0 mg, 0.416 mmol, 1.2 equiv) in Pyridine (5 mL) were added EDCI (99.7 mg, 0.520 mmol, 1.5 equiv) at room temperature. The resulting mixture was stirred for 2 h at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse flash chromatography to afford 2-(2,6-dioxopiperidin-3-yl)-N-(isoquinolin-4-yl)-1-oxoisoindoline-5-carboxamide (36 mg, 25%) as a white solid.

LC-MS (ES, m/z)=415.10 [M+1]⁺. ¹H NMR (400 MHz, DMSO-d₆) δ 10.72 (s, 2H), 9.28 (s, 1H), 8.65 (s, 1H), 8.34-8.29 (m, 1H), 8.27-8.18 (m, 2H), 8.04 (d, J=8.4 Hz, 1H), 7.93 (d, J 7.9 Hz, 1H), 7.84 (ddd, J=8.4, 6.9, 1.3 Hz, 1H), 7.75 (ddd, J=8.1, 6.8, 1.2 Hz, 1H), 5.19 (dd, J=13.3, 5.1 Hz, 1H), 4.60 (d, J=17.6 Hz, 1H), 4.47 (d, J=17.6 Hz, 1H), 2.94 (ddd, J=17.8, 13.6, 5.4 Hz, 1H), 2.68-2.59 (m, 1H), 2.48-2.39 (m, 1H), 2.07 (s, 1H).

### Example 3

### Synthesis of 2-(2,6-dioxopiperidin-3-yl)-N-(isoquinolin-1-yl)-1-oxoisoindoline-5-carboxamide

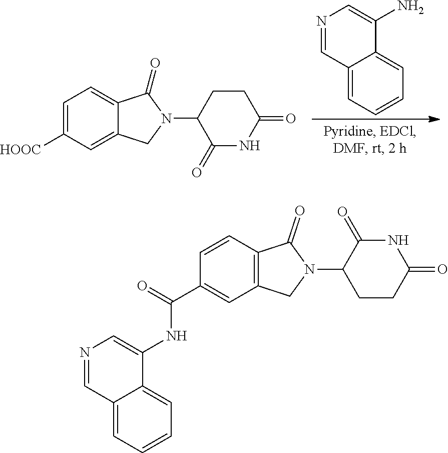 [SMILES: O=C1CCC(N2Cc3cc(C(=O)O)ccc3C2=O)C(=O)N1 ; Nc1cncc2ccccc12 ; O=C1CCC(N2Cc3cc(C(=O)Nc4cncc5ccccc45)ccc3C2=O)C(=O)N1]

Step 1. Preparation of 2-(2,6-dioxopiperidin-3-yl)-N-(isoquinolin-1-yl)-1-oxoisoindoline-5-carboxamide. To a stirred mixture of 2-(2,6-dioxopiperidin-3-yl)-1-oxoisoindoline-5-carboxylic acid (100 mg, 0.347 mmol, 1 equiv) and 1-isoquinolinamine (60.0 mg, 0.416 mmol, 1.2 equiv) in Pyridine (5 mL) were added EDCI (99.8 mg, 0.520 mmol, 1.5 equiv) at room temperature. The resulting mixture was stirred for 2 h at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse-phase flash chromatography to afford 2-(2,6-dioxopiperidin-3-yl)-N-(isoquinolin-1-yl)-1-oxoisoindoline-5-carboxamide (30 mg, 20%) as a white solid. LC-MS (ES, m/z)=415.05 [M+1]⁺. ¹H NMR (400 MHz, DMSO-d₆) δ 11.03 (s, 1H), 8.06-7.57 (m, 6H), 5.18 (dd, J=13.3, 5.2 Hz, 1H), 4.59 (d, J=17.5 Hz, 1H), 4.46 (d, J=17.5 Hz, 1H), 2.94 (ddd, J=18.0, 13.7, 5.5 Hz, 1H), 2.63 (d, J=17.6 Hz, 1H), 2.49-2.38 (m, 1H), 2.06 (d, J=8.5 Hz, 1H).

### Example 4

### Synthesis of 2-(2,6-dioxopiperidin-3-yl)-1-oxo-N—((R)-1,2,3,4-tetrahydronaphthalen-1-yl)isoindoline-5-carboxamide

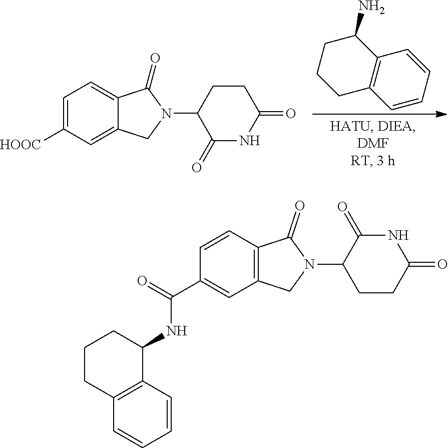 [SMILES: O=C1CCC(N2Cc3cc(C(=O)O)ccc3C2=O)C(=O)N1 ; N[C@@H]1CCCc2ccccc21 ; O=C1CCC(N2Cc3cc(C(=O)N[C@@H]4CCCc5ccccc54)ccc3C2=O)C(=O)N1]

Step 1. Preparation of 2-(2,6-dioxopiperidin-3-yl)-1-oxo-N—((R)-1,2,3,4-tetrahydronaphthalen-1-yl)isoindoline-5-carboxamide. To a stirred solution of 2-(2,6-dioxopiperidin-3-yl)-1-oxoisoindoline-5-carboxylic acid (150 mg, 0.520 mmol, 1 equiv) and (1R)-1,2,3,4-tetrahydronaphthalen-1-amine (84.3 mg, 0.572 mmol, 1.1 equiv) in DMF (2 ml) were added DIEA (201.8 mg, 1.560 mmol, 3 equiv) and HATU (296.8 mg, 0.780 mmol, 1.5 equiv) at room temperature under air atmosphere. The resulting mixture was stirred for 3 h at room temperature under air atmosphere. The resulting mixture was extracted with EtOAc (3×10 mL). The combined organic layers were washed with NaCl(aq) (3×3 mL), dried over anhydrous Na₂SO₄. After filtration, the filtrate was concentrated under reduced pressure. The resulting mixture was concentrated under reduced pressure. The residue was purified by reverse-phase flash chromatography to afford 2-(2,6-dioxopiperidin-3-yl)-1-oxo-N—((R)-1,2,3,4-tetrahydronaphthalen-1-yl)isoindoline-5-carboxamide (58 mg, 26%) as an off-white solid. LC-MS (ES, m/z)=418.10 [M+1]⁺. ¹H NMR (400 MHz, DMSO-d₆) δ 10.99 (s, 1H), 8.96 (d, J=8.5 Hz, 1H), 8.12 (s, 2H), 8.03 (dd, J=7.9, 1.4 Hz, 1H), 7.80 (d, J=8.0 Hz, 4H), 5.26 (td, J=8.4, 4.7 Hz, 1H), 5.14 (dd, J=13.3, 5.1 Hz, 1H), 4.51 (dd, J=17.7, 2.2 Hz, 1H), 4.39 (dd, J=17.6, 2.1 Hz, 1H), 2.92 (ddd, J=17.3, 13.6, 5.4 Hz, 3H), 2.78 (q, J=7.3, 5.8 Hz, 1H), 2.66-2.56 (m, 1H), 2.42 (qd, J=13.4, 4.8 Hz, 3H), 2.10-1.93 (m, 2H).

### Example 5

### Synthesis of 2-(2,6-dioxopiperidin-3-yl)-1-oxo-N—((S)-1,2,3,4-tetrahydronaphthalen-1-yl)isoindoline-5-carboxamide

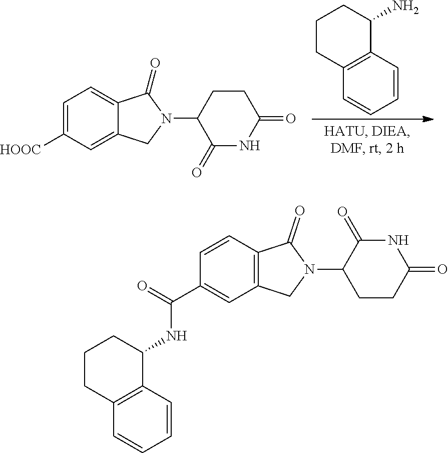 [SMILES: O=C1CCC(N2Cc3cc(C(=O)O)ccc3C2=O)C(=O)N1 ; N[C@H]1CCCc2ccccc21 ; O=C1CCC(N2Cc3cc(C(=O)N[C@H]4CCCc5ccccc54)ccc3C2=O)C(=O)N1]

Step 1. Preparation of 2-(2,6-dioxopiperidin-3-yl)-1-oxo-N—((S)-1,2,3,4-tetrahydronaphthalen-1-yl)isoindoline-5-carboxamide. To a stirred mixture of 2-(2,6-dioxopiperidin-3-yl)-1-oxoisoindole-5-carboxylic acid (100 mg, 0.347 mmol, 1 equiv) and (1S)-1,2,3,4-tetrahydronaphthalen-1-amine (102.1 mg, 0.694 mmol, 2 equiv) in DMF (1 mL) were added HATU (197.8 mg, 0.520 mmol, 1.5 equiv) and DIEA (134.5 mg, 1.041 mmol, 3 equiv) at room temperature. The resulting mixture was stirred for 2 h at room temperature. The resulting mixture was extracted with EtOAc (3×10 mL). The combined organic layers were washed with brine (2×5 mL), dried over anhydrous Na₂SO₄. After filtration, the filtrate was concentrated under reduced pressure. The residue was purified by reverse-phase flash chromatography to afford 2-(2,6-dioxopiperidin-3-yl)-1-oxo-N—[(S)-1,2,3,4-tetrahydronaphthalen-1-yl]isoindole-5-carboxamide (42 mg, 29%) as a white solid.

LC-MS (ES, m/z)=418.15 [M+1]⁺. ¹H NMR (400 MHz, DMSO-d₆) δ 11.00 (s, 1H), 8.96 (d, J=8.6 Hz, 1H), 8.12 (s, 1H), 8.03 (d, J=8.0 Hz, 1H), 7.80 (d, J=8.0 Hz, 1H), 7.24-7.18 (m, 1H), 7.16 (dd, J=8.4, 6.4 Hz, 3H), 5.26 (d, J=5.0 Hz, 1H), 5.14 (dd, J=13.3, 5.1 Hz, 1H), 4.51 (d, J=17.4 Hz, 1H), 4.39 (d, J=17.5 Hz, 1H), 2.96-2.86 (m, 1H), 2.78 (d, J=6.2 Hz, 1H), 2.61 (d, J=17.1 Hz, 1H), 2.47-2.36 (m, 1H), 2.10-1.95 (m, 3H), 1.91-1.73 (m, 2H).

### Example 6

### Synthesis of N—((R)-chroman-4-yl)-2-(2,6-dioxopiperidin-3-yl)-1-oxoisoindoline-5-carboxamide

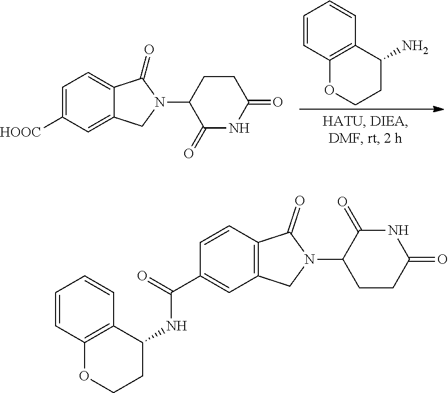 [SMILES: O=C1CCC(N2Cc3cc(C(=O)O)ccc3C2=O)C(=O)N1 ; N[C@@H]1CCOc2ccccc21 ; O=C1CCC(N2Cc3cc(C(=O)N[C@@H]4CCOc5ccccc54)ccc3C2=O)C(=O)N1]

Step 1. Preparation of N—((R)-chroman-4-yl)-2-(2,6-dioxopiperidin-3-yl)-1-oxoisoindoline-5-carboxamide. To a stirred mixture of 2-(2,6-dioxopiperidin-3-yl)-1-oxoisoindole-5-carboxylic acid (90 mg, 0.312 mmol, 1 equiv) and (4R)-3,4-dihydro-2H-1-benzopyran-4-amine (93.1 mg, 0.624 mmol, 2 equiv) in DMF (1 mL) were added DIEA (121.0 mg, 0.936 mmol, 3 equiv) and HATU (178.0 mg, 0.468 mmol, 1.5 equiv) at room temperature. The resulting mixture was stirred for 2 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford N—((R)-chroman-4-yl)-2-(2,6-dioxopiperidin-3-yl)-1-oxoisoindoline-5-carboxamide (57 mg, 43%) as a white solid.

LC-MS (ES, m/z)=420.05 [M+1]⁺. ¹H NMR (400 MHz, DMSO-d₆) δ 11.00 (s, 1H), 9.06 (d, J=8.1 Hz, 1H), 8.13 (s, 1H), 8.07-8.00 (m, 1H), 7.81 (d, J=8.0 Hz, 1H), 7.23-7.12 (m, 2H), 6.88 (td, J=7.5, 1.3 Hz, 1H), 6.81 (dd, J=8.2, 1.2 Hz, 1H), 5.30 (q, J=6.8 Hz, 1H), 5.14 (dd, J=13.3, 5.1 Hz, 1H), 4.52 (dd, J=17.8, 2.6 Hz, 1H), 4.44-4.35 (m, 1H), 4.28 (ddt, J=15.0, 11.4, 7.7 Hz, 2H), 2.92 (ddd, J=18.2, 13.7, 5.6 Hz, 1H), 2.61 (d, J=17.1 Hz, 1H), 2.47-2.36 (m, 1H), 2.15-2.11 (m, 1H), 2.11-1.99 (m, 2H).

### Example 7

### Synthesis of N—((S)-chroman-4-yl)-2-(2,6-dioxopiperidin-3-yl)-1-oxoisoindoline-5-carboxamide

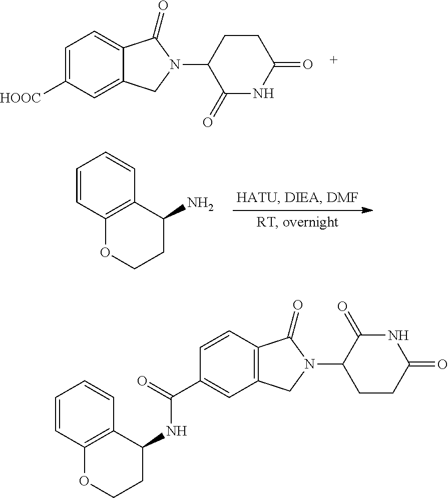 [SMILES: O=C1CCC(N2Cc3cc(C(=O)O)ccc3C2=O)C(=O)N1 ; N[C@H]1CCOc2ccccc21 ; O=C1CCC(N2Cc3cc(C(=O)N[C@H]4CCOc5ccccc54)ccc3C2=O)C(=O)N1]

Step 1. Preparation of N—((S)-chroman-4-yl)-2-(2,6-dioxopiperidin-3-yl)-1-oxoisoindoline-5-carboxamide. A solution of 2-(2,6-dioxopiperidin-3-yl)-1-oxoisoindole-5-carboxylic acid (50 mg, 0.173 mmol, 1 equiv) and (4S)-3,4-dihydro-2H-1-benzopyran-4-amine (80.6 mg, 0.540 mmol, 3.11 equiv), DIEA (90 mg, 0.696 mmol, 4.01 equiv) in dimethylformamide (1 mL) was stirred for 1 min at room temperature. To the above mixture was added HATU (99 mg, 0.260 mmol, 1.50 equiv) in portions over 2 min at room temperature. The resulting mixture was stirred for additional overnight at room temperature. The resulting mixture was concentrated under reduced pressure. The residue was purified by reverse-phase flash chromatography to afford N—((S)-chroman-4-yl)-2-(2,6-dioxopiperidin-3-yl)-1-oxoisoindoline-5-carboxamide (18 mg, 25%) as an off-white solid.

LC-MS (ES, m/z)=418.05 [M−1]⁻. ¹H NMR (400 MHz, DMSO-d₆) δ 11.01 (s, 1H), 9.07 (d, J=8.1 Hz, 1H), 8.15-7.88 (m, 2H), 7.81 (d, J=7.9 Hz, 1H), 7.24-7.11 (m, 2H), 6.93-6.74 (m, 2H), 5.31 (q, J=6.7 Hz, 1H), 5.14 (dd, J=13.3, 5.1 Hz, 1H), 4.59-4.17 (m, 4H), 2.92 (ddd, J=18.1, 13.5, 5.4 Hz, 1H), 2.61 (dt, J=15.7, 3.2 Hz, 1H), 2.42 (tt, J=13.2, 6.6 Hz, 1H), 2.29-1.80 (m, 3H).

### Example 8

### Synthesis of N-(benzo[b]thiophen-3-yl)-2-(2,6-dioxopiperidin-3-yl)-1-oxoisoindoline-5-carboxamide

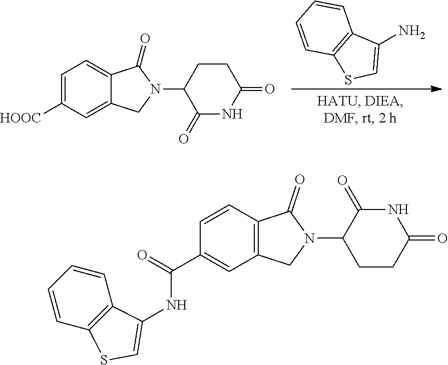 [SMILES: O=C1CCC(N2Cc3cc(C(=O)O)ccc3C2=O)C(=O)N1 ; Nc1csc2ccccc12 ; O=C1CCC(N2Cc3cc(C(=O)Nc4csc5ccccc45)ccc3C2=O)C(=O)N1]

Step 1. Preparation of N-(benzo[b]thiophen-3-yl)-2-(2,6-dioxopiperidin-3-yl)-1-oxoisoindoline-5-carboxamide. To a stirred mixture of 1-benzothiophen-3-amine (154.8 mg, 1.037 mmol, 1 equiv) and 2-(2,6-dioxopiperidin-3-yl)-1-oxoisoindole-5-carboxylic acid (299.0 mg, 1.037 mmol, 1 equiv) in DMF (3 mL) were added DIEA (402.2 mg, 3.111 mmol, 3 equiv) and HATU (591.7 mg, 1.555 mmol, 1.5 equiv) at room temperature. The resulting mixture was stirred for 2 h at room temperature. The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by reverse-phase flash chromatography to afford N-(benzo[b]thiophen-3-yl)-2-(2,6-dioxopiperidin-3-yl)-1-oxoisoindoline-5-carboxamide (50 mg, 12%) as an off-white solid.

LC-MS (ES, m/z)=418.05 [M−1]⁻. ¹H NMR (400 MHz, DMSO-d₆) δ 11.03 (s, 1H), 10.62 (s, 1H), 8.25 (s, 1H), 8.14 (t, J=7.5 Hz, 2H), 8.07-7.97 (m, 2H), 7.90 (d, J=7.9 Hz, 1H), 7.49-7.39 (m, 2H), 5.18 (dd, J=13.2, 5.2 Hz, 1H), 4.58 (d, J=17.5 Hz, 1H), 4.46 (d, J=17.5 Hz, 1H), 3.00-2.88 (m, 1H), 2.63 (d, J=17.8 Hz, 1H), 2.46 (d, J=8.6 Hz, 1H), 2.10-2.02 (m, 1H).

### Example 9

### Synthesis of 2-(2,6-dioxopiperidin-3-yl)-1-oxo-N-(quinolin-4-yl)isoindoline-5-carboxamide

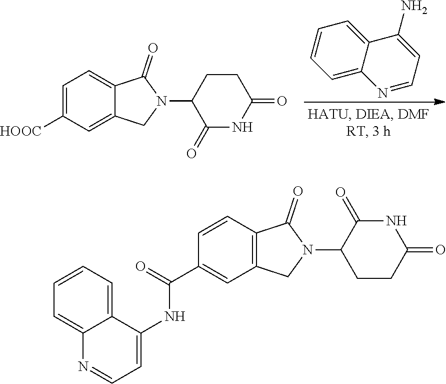 [SMILES: O=C1CCC(N2Cc3cc(C(=O)O)ccc3C2=O)C(=O)N1 ; Nc1ccnc2ccccc12 ; O=C1CCC(N2Cc3cc(C(=O)Nc4ccnc5ccccc45)ccc3C2=O)C(=O)N1]

Step 1. Preparation of 2-(2,6-dioxopiperidin-3-yl)-1-oxo-N-(quinolin-4-yl)isoindoline-5-carboxamide. To a stirred solution of 2-(2,6-dioxopiperidin-3-yl)-1-oxoisoindole-5-carboxylic acid (150 mg, 0.520 mmol, 1 equiv) and 4-aminoquinoline (82.5 mg, 0.572 mmol, 1.1 equiv) in DMF (2 ml) were added DIEA (201.8 mg, 1.560 mmol, 3 equiv) and HATU (296.8 mg, 0.780 mmol, 1.5 equiv) at room temperature. The resulting mixture was stirred for 3 h at room temperature. The resulting mixture was extracted with EtOAc (3×10 mL). The combined organic layers were washed with NaCl(aq) (3×3 mL), dried over anhydrous Na₂SO₄. After filtration, the filtrate was concentrated under reduced pressure. The resulting mixture was concentrated under reduced pressure. The crude product was purified by Prep-HPLC to afford 2-(2,6-dioxopiperidin-3-yl)-1-oxo-N-(quinolin-4-yl)isoindoline-5-carboxamide (7.3 mg, 3%) as an off-white solid.

LC-MS (ES, m/z)=415.20 [M+1]⁺. ¹H NMR (400 MHz, DMSO-d₆) δ 11.02 (d, J=12.9 Hz, 1H), 10.86 (s, 1H), 8.91 (d, J=4.9 Hz, 1H), 8.30 (d, J=9.2 Hz, 2H), 8.21 (d, J=8.0 Hz, 1H), 8.13-8.00 (m, 1H), 7.99-7.89 (m, 2H), 7.85-7.71 (m, 1H), 7.65 (t, J=7.6 Hz, 1H), 5.17 (td, J=15.5, 14.4, 6.7 Hz, 1H), 4.60 (d, J=17.6 Hz, 3H), 3.04-2.85 (m, 2H), 2.71-2.56 (m, 2H), 2.53-2.36 (m, 1H), 2.12-1.98 (m, 1H).

### Example 10

### Synthesis of 3-(1-oxo-5-(pyrrolidine-1-carbonyl)isoindolin-2-yl)piperidine-2,6-dione

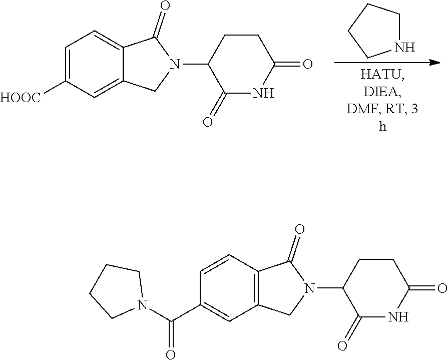 [SMILES: O=C1CCC(N2Cc3cc(C(=O)O)ccc3C2=O)C(=O)N1 ; C1CCNC1 ; O=C1CCC(N2Cc3cc(C(=O)N4CCCC4)ccc3C2=O)C(=O)N1]

Step 1. Preparation of 3-(1-oxo-5-(pyrrolidine-1-carbonyl)isoindolin-2-yl)piperidine-2,6-dione. To a stirred mixture of 2-(2,6-dioxopiperidin-3-yl)-1-oxoisoindole-5-carboxylic acid (150 mg, 0.520 mmol, 1 equiv) and pyrrolidine (40.7 mg, 0.572 mmol, 1.1 equiv) in DMF (2 ml) were added DIEA (201.8 mg, 1.560 mmol, 3 equiv) and HATU (296.79 mg, 0.780 mmol, 1.5 equiv) at room temperature under nitrogen atmosphere. The resulting mixture was stirred for additional 3 h at room temperature. The resulting mixture was extracted with EtOAc (3×10 mL). The combined organic layers were washed with NaCl(aq) (3×3 mL), dried over anhydrous Na₂SO₄. After filtration, the filtrate was concentrated under reduced pressure. The residue was purified by reverse-phase flash chromatography to afford 3-(1-oxo-5-(pyrrolidine-1-carbonyl)isoindolin-2-yl)piperidine-2,6-dione (100 mg crude) as an off-white solid. The residue was dissolved in DMF (2 mL). The crude product was further purified by Prep-HPLC to afford 3-(1-oxo-5-(pyrrolidine-1-carbonyl)isoindolin-2-yl)piperidine-2,6-dione (39 mg, 22%) as an off-white solid. LC-MS (ES, m/z)=342.15 [M+1]⁺. ¹H NMR (400 MHz, DMSO-d₆) δ 11.01 (s, 1H), 7.80-7.71 (m, 1H), 7.62 (dd, J=7.8, 1.4 Hz, 1H), 7.55 (d, j=7.9, 1.5 Hz, 1H), 5.14 (dd, J=13.3, 5.1 Hz, 1H), 4.50 (d, J=17.5 Hz, 1H), 4.37 (d, J=17.5 Hz, 1H), 3.49 (t, J=6.8 Hz, 2H), 3.36 (d, J=12.8 Hz, 2H), 2.92 (ddd, J=17.3, 13.6, 5.4 Hz, 1H), 2.61 (ddd, J=17.3, 4.4, 2.3 Hz, 1H), 2.41 (qd, J=13.2, 4.4 Hz, 1H), 2.08-1.97 (m, 1H), 1.86 (dp, J=20.1, 6.8 Hz, 4H).

### Example 11

### Synthesis of 3-(5-(indoline-1-carbonyl)-1-oxoisoindolin-2-yl)piperidine-2,6-dione

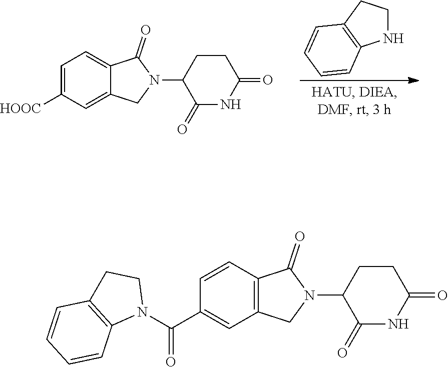

Step 1. Preparation of 3-(5-(indoline-1-carbonyl)-1-oxoisoindolin-2-yl)piperidine-2,6-dione. To a stirred mixture of 2-(2,6-dioxopiperidin-3-yl)-1-oxoisoindole-5-carboxylic acid (150 mg, 0.520 mmol, 1 equiv) and indoline (93.0 mg, 0.780 mmol, 1.5 equiv) in DMF (2 mL, 25.843 mmol, 49.66 equiv) was added DIEA (201.8 mg, 1.560 mmol, 3 equiv) and HATU (296.8 mg, 0.780 mmol, 1.5 equiv) at room temperature. The resulting mixture was stirred for 3 h at room temperature. The resulting mixture was extracted with EtOAc (3×50 mL). The combined organic layers were washed with brine (2×30 mL), dried over anhydrous Na₂SO₄. After filtration, the filtrate was concentrated under reduced pressure. The resulting mixture was concentrated under reduced pressure. The residue was purified by reverse-phase flash chromatography to afford 3-(5-(indoline-1-carbonyl)-1-oxoisoindolin-2-yl)piperidine-2,6-dione (23 mg, 11%) as a white solid.

LC-MS (ES, m/z)=390 [M+1]⁺. ¹H NMR (400 MHz, DMSO-d₆) δ 11.02 (s, 1H), 7.87-7.80 (m, 2H), 7.71 (d, J=8.0 Hz, 1H), 7.29 (d, J=7.3 Hz, 1H), 7.20 (s, 1H), 7.07 (s, 1H), 5.16 (dd, J=13.3, 5.0 Hz, 1H), 4.54 (d, J=17.5 Hz, 1H), 4.41 (d, J=17.5 Hz, 1H), 4.00 (s, 1H), 3.10 (t, J=8.3 Hz, 2H), 2.93 (ddd, J=17.8, 13.6, 5.5 Hz, 1H), 2.61 (d, J=17.1 Hz, 1H), 2.43 (dd, J=13.2, 4.6 Hz, 1H), 2.08-2.00 (m, 1H).

### Example 12

### Synthesis of 3-(5-(isoindoline-2-carbonyl)-1-oxoisoindolin-2-yl)piperidine-2,6-dione

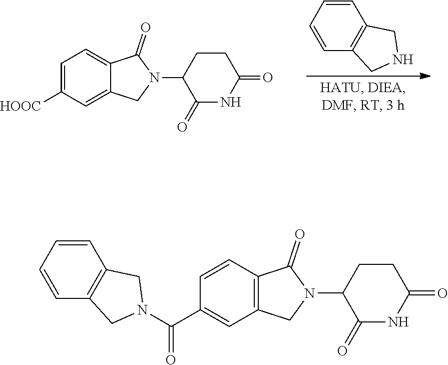 [SMILES: O=C1CCC(N2Cc3cc(C(=O)O)ccc3C2=O)C(=O)N1 ; c1ccc2c(c1)CNC2 ; O=C1CCC(N2Cc3cc(C(=O)N4Cc5ccccc5C4)ccc3C2=O)C(=O)N1]

Step 1. Preparation of 3-(5-(isoindoline-2-carbonyl)-1-oxoisoindolin-2-yl)piperidine-2,6-dione. To a stirred mixture of 2-(2,6-dioxopiperidin-3-yl)-1-oxoisoindole-5-carboxylic acid (200 mg, 0.694 mmol, 1 equiv) and isoindoline (90.95 mg, 0.763 mmol, 1.1 equiv) in DMF (3 mL) were added DIEA (0.60 mL, 3.470 mmol, 5 equiv) and HATU (395.7 mg, 1.041 mmol, 1.5 equiv) at room temperature under nitrogen atmosphere. The resulting mixture was stirred for additional 3 h at room temperature. The resulting mixture was extracted with EtOAc (3×10 mL). The combined organic layers were washed with NaCl (aq) (3×3 mL), dried over anhydrous Na₂SO₄. After filtration, the filtrate was concentrated under reduced pressure. The resulting mixture was concentrated under reduced pressure. The residue was purified by reverse-phase flash chromatography to afford 3-(5-(isoindoline-2-carbonyl)-1-oxoisoindolin-2-yl)piperidine-2,6-dione (51 mg, 19%) as an off-white solid.

LC-MS (ES, m/z)=390.15 [M+1]⁺. ¹H NMR (400 MHz, DMSO-d₆) δ 11.03 (s, 1H), 7.87-7.80 (m, 2H), 7.72 (dd, J=7.8, 1.3 Hz, 1H), 7.41 (d, J=7.2 Hz, 1H), 7.36-7.22 (m, 3H), 5.16 (dd, J=13.3, 5.1 Hz, 1H), 4.90 (s, 2H), 4.76 (s, 2H), 4.53 (d, J=17.6 Hz, 1H), 4.40 (d, J=17.5 Hz, 1H), 3.00-2.86 (m, 1H), 2.62 (ddd, J=17.2, 4.5, 2.2 Hz, 1H), 2.43 (qd, J=13.2, 4.4 Hz, 1H), 2.10-1.98 (m, 1H).

### Example 13

### Synthesis of 3-(1-oxo-5-(piperidine-1-carbonyl)isoindolin-2-yl)piperidine-2,6-dione

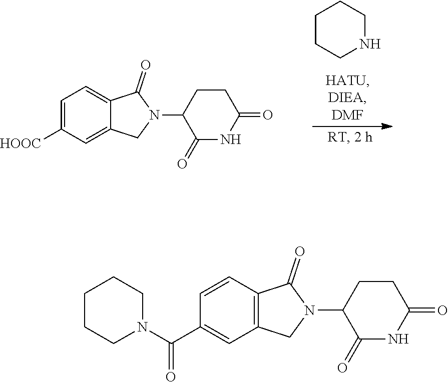 [SMILES: C1CCNCC1 ; O=C1CCC(N2Cc3cc(C(=O)O)ccc3C2=O)C(=O)N1 ; O=C1CCC(N2Cc3cc(C(=O)N4CCCCC4)ccc3C2=O)C(=O)N1]

Step 1. Preparation of 3-(1-oxo-5-(piperidine-1-carbonyl)isoindolin-2-yl)piperidine-2,6-dione. To a stirred solution of 2-(2,6-dioxopiperidin-3-yl)-1-oxoisoindole-5-carboxylic acid (100 mg, 0.347 mmol, 1 equiv) and piperidine (35.5 mg, 0.416 mmol, 1.2 equiv) in DMF (2 mL, 25.843 mmol, 74.50 equiv) was added DIEA (134.5 mg, 1.041 mmol, 3 equiv) at room temperature. The resulting mixture was stirred for 10 min at room temperature. To the above mixture was added HATU (197.9 mg, 0.520 mmol, 1.5 equiv) at room temperature. The resulting mixture was stirred for additional 2 h at room temperature. The residue was purified by reversed-phase flash chromatography with the following conditions: column, C18 silica gel; mobile phase, MeCN in Water (0.1% FA), 0% to 100% gradient in 10 min; detector, UV 254 nm. The crude product was purified by Prep-HPLC to afford 3-(1-oxo-5-(piperidine-1-carbonyl)isoindolin-2-yl)piperidine-2,6-dione (36 mg, 29%) as a white solid.

LC-MS (ES, m/z)=356 [M+1]⁺. ¹H NMR (400 MHz, DMSO-d₆) δ 11.01 (s, 1H), 7.78 (d, J 7.8 Hz, 1H), 7.61 (s, 1H), 7.48 (dd, J=7.8, 1.4 Hz, 1H), 5.14 (dd, J=13.3, 5.1 Hz, 1H), 4.50 (d, J=17.5 Hz, 1H), 4.37 (d, J=17.5 Hz, 1H), 3.61 (s, 2H), 3.25 (s, 2H), 2.92 (ddd, J=17.3, 13.6, 5.4 Hz, 1H), 2.60 (dt, J=17.1, 3.2 Hz, 1H), 2.41 (qd, J=13.2, 4.5 Hz, 1H), 2.10-1.96 (m, 1H), 1.70-1.39 (m, 6H).

### Example 14

### Synthesis of 3-(1-oxo-5-(3-oxopiperazine-1-carbonyl)isoindolin-2-yl)piperidine-2,6-dione

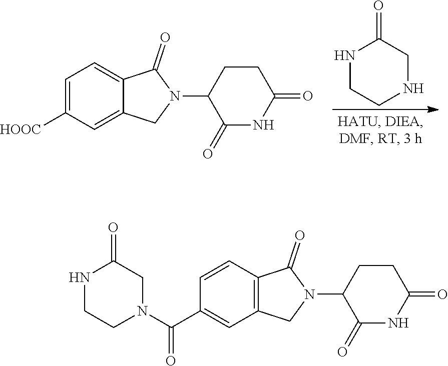

Step 1. Preparation of 3-(1-oxo-5-(3-oxopiperazine-1-carbonyl)isoindolin-2-yl)piperidine-2,6-dione. To a stirred mixture of 2-(2,6-dioxopiperidin-3-yl)-1-oxoisoindole-5-carboxylic acid (300 mg, 1.041 mmol, 1 equiv) and piperazin-2-one (156.3 mg, 1.561 mmol, 1.5 equiv) in DMF (3 mL, 38.765 mmol, 37.25 equiv) were added DIEA (403.5 mg, 3.123 mmol, 3 equiv) and HATU (593.6 mg, 1.561 mmol, 1.5 equiv) at room temperature. The resulting mixture was stirred for 3 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The resulting mixture was concentrated under reduced pressure. The residue was purified by reverse-phase flash chromatography to afford 3-(1-oxo-5-(3-oxopiperazine-1-carbonyl)isoindolin-2-yl)piperidine-2,6-dione (83 mg, 21%) as a white solid.

LC-MS (ES, m/z)=371.05 [M+1]⁺. ¹H NMR (400 MHz, DMSO-d₆) δ 11.01 (s, 1H), 8.15 (d, J=2.6 Hz, 1H), 7.80 (d, J=7.7 Hz, 1H), 7.69 (s, 1H), 7.57 (dd, J=7.8, 1.4 Hz, 1H), 5.14 (dd, J=13.3, 5.1 Hz, 1H), 4.52 (d, J=17.5 Hz, 1H), 4.39 (d, J=17.5 Hz, 1H), 4.12 (s, 1H), 3.89 (s, 1H), 3.80 (s, 1H), 3.48 (s, 1H), 3.22 (s, 2H), 2.92 (ddd, J=17.2, 13.6, 5.4 Hz, 1H), 2.61 (ddd, J=17.3, 4.4, 2.3 Hz, 1H), 2.41 (qd, J=13.2, 4.5 Hz, 1H), 2.10-1.96 (m, 1H).

### Example 15

### Synthesis of 3-(5-(4-methylpiperazine-1-carbonyl)-1-oxoisoindolin-2-yl)piperidine-2,6-dione

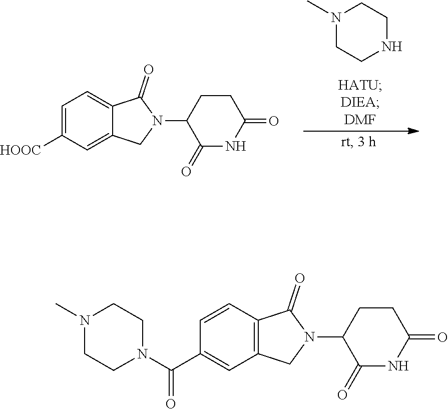

Step 1. Preparation of 3-(5-(4-methylpiperazine-1-carbonyl)-1-oxoisoindolin-2-yl)piperidine-2,6-dione. To a stirred mixture of 2-(2,6-dioxopiperidin-3-yl)-1-oxoisoindole-5-carboxylic acid (200 mg, 0.694 mmol, 1 equiv) and piperazine, 1-methyl-(104.2 mg, 1.041 mmol, 1.5 equiv) in DMF (2 mL, 25.843 mmol, 37.25 equiv) were added DIEA (269.0 mg, 2.082 mmol, 3 equiv) and HATU (395.7 mg, 1.041 mmol, 1.5 equiv) at room temperature. The resulting mixture was stirred for 3 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The resulting mixture was concentrated under reduced pressure. The residue was purified by reverse-phase flash chromatography to afford 3-(5-(4-methylpiperazine-1-carbonyl)-1-oxoisoindolin-2-yl)piperidine-2,6-dione (66 mg, 25%) as a white oil.

LC-MS (ES, m/z)=371.25 [M+1]⁺. ¹H NMR (400 MHz, DMSO-d₆) δ 7.83 (d, J=7.8 Hz, 1H), 7.70 (s, 1H), 7.59 (dd, J=7.8, 1.4 Hz, 1H), 5.15 (dd, J=13.3, 5.1 Hz, 1H), 4.54 (s, 1H), 4.50 (s, 1H), 4.40 (d, J=17.6 Hz, 1H), 3.13 (d, J=13.1 Hz, 3H), 2.93 (ddd, J=17.3, 13.6, 5.4 Hz, 1H), 2.84 (s, 3H), 2.62 (ddd, J=17.2, 4.5, 2.3 Hz, 1H), 2.42 (qd, J=13.1, 4.4 Hz, 3H), 2.10-1.97 (m, 1H).

### Example 16

### Synthesis of 3-(5-(4-methyl-3-oxopiperazine-1-carbonyl)-1-oxoisoindolin-2-yl)piperidine-2,6-dione

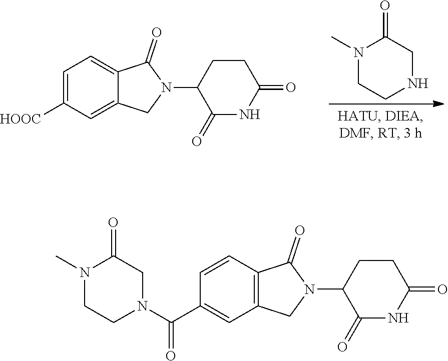

Step 1. Preparation of 3-(5-(4-methyl-3-oxopiperazine-1-carbonyl)-1-oxoisoindolin-2-yl)piperidine-2,6-dione. To a stirred solution of 2-(2,6-dioxopiperidin-3-yl)-1-oxoisoindole-5-carboxylic acid (150 mg, 0.520 mmol, 1 equiv) and 1-methylpiperazin-2-one (65.3 mg, 0.572 mmol, 1.1 equiv) in DMF (2 ml) were added DIEA (201.8 mg, 1.560 mmol, 3 equiv) and HATU (296.8 mg, 0.780 mmol, 1.5 equiv) at room temperature under nitrogen atmosphere. The resulting mixture was stirred for additional 3 h at room temperature. The resulting mixture was extracted with EtOAc (3×10 mL). The combined organic layers were washed with NaCl (aq) (3×3 mL), dried over anhydrous MgSO₄. After filtration, the filtrate was concentrated under reduced pressure. The resulting mixture was concentrated under reduced pressure. The residue was purified by reverse-phase flash chromatography to afford 3-(5-(4-methyl-3-oxopiperazine-1-carbonyl)-1-oxoisoindolin-2-yl)piperidine-2,6-dione (65 mg, 31%) as an off-white solid.

LC-MS (ES, m/z)=385.10 [M+1]⁺. ¹H NMR (400 MHz, DMSO-d₆) δ 11.01 (s, 1H), 7.81 (d, J=7.8 Hz, 1H), 7.69 (s, 1H), 7.57 (dd, J=7.7, 1.3 Hz, 1H), 5.14 (dd, J=13.3, 5.1 Hz, 1H), 4.52 (d, J=17.6 Hz, 1H), 4.39 (d, J=17.4 Hz, 1H), 4.17 (s, 1H), 3.91 (s, 2H), 3.7 (S, 1H), 3.57 (S, 1H), 3.4 (S, 1H), 2.99-2.87 (m, 3H), 2.61 (ddd, J=17.3, 4.6, 2.3 Hz, 1H), 2.41 (qd, J=13.2, 4.4 Hz, 1H), 2.02 (dtd, J=12.8, 5.4, 2.3 Hz, 1H).

### Example 17

### Synthesis of 3-(5-(3,4-dihydro-2H-benzo[b][1,4]oxazine-4-carbonyl)-1-oxoisoindolin-2-yl)piperidine-2,6-dione

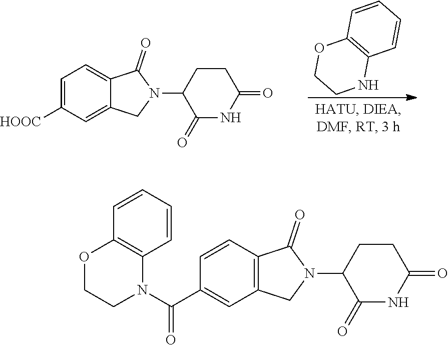 [SMILES: O=C1CCC(N2Cc3cc(C(=O)O)ccc3C2=O)C(=O)N1 ; c1ccc2c(c1)NCCO2 ; O=C1CCC(N2Cc3cc(C(=O)N4CCOc5ccccc54)ccc3C2=O)C(=O)N1]

Step 1. Preparation of 3-(5-(3,4-dihydro-2H-benzo[b][1,4]oxazine-4-carbonyl)-1-oxoisoindolin-2-yl)piperidine-2,6-dione. To a stirred solution of 2-(2,6-dioxopiperidin-3-yl)-1-oxoisoindole-5-carboxylic acid (150 mg, 0.520 mmol, 1 equiv) and 3,4-dihydro-2H-1,4-benzoxazine (77.4 mg, 0.572 mmol, 1.1 equiv) in DMF (2 ml) were added DIEA (201.8 mg, 1.560 mmol, 3 equiv) and HATU (296.8 mg, 0.780 mmol, 1.5 equiv) at room temperature under nitrogen atmosphere. The resulting mixture was stirred for additional 3 h at room temperature. The resulting mixture was extracted with EtOAc (3×10 mL). The combined organic layers were washed with NaCl (3×3 mL), dried over anhydrous MgSO₄. After filtration, the filtrate was concentrated under reduced pressure. The resulting mixture was concentrated under reduced pressure. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 23% B to 38% B in 8 min; Wave Length: 254/220 nm; RT1 (min): 7.30) to afford 3-(5-(3,4-dihydro-2H-benzo[b][1,4]oxazine-4-carbonyl)-1-oxoisoindolin-2-yl)piperidine-2,6-dione (28 mg, 13%) as an off-white solid.

LC-MS (ES, m/z)=406.15 [M+1]⁺. ¹H NMR (400 MHz, DMSO-d₆) δ 11.02 (s, 1H), 7.83-7.74 (m, 1H), 7.63 (d, J=7.8 Hz, 1H), 7.26 (s, 1H), 7.02 (td, J=7.7, 7.2, 1.5 Hz, 1H), 6.92 (dd, J=8.2, 1.5 Hz, 1H), 6.75 (t, J=7.7 Hz, 1H), 5.14 (dd, J=13.3, 5.1 Hz, 1H), 4.50 (d, J=17.6 Hz, 1H), 4.38 (d, J=17.6 Hz, 1H), 4.33 (d, J=4.7 Hz, 3H), 3.87 (d, J=4.3 Hz, 2H), 2.92 (ddd, J=17.3, 13.6, 5.4 Hz, 1H), 2.61 (ddd, J=17.2, 4.6, 2.3 Hz, 1H), 2.41 (qd, J=13.1, 4.4 Hz, 1H), 2.10-1.97 (m, 1H).

### Example 18

### Synthesis of 3-(1-oxo-5-(piperazine-1-carbonyl)isoindolin-2-yl)piperidine-2,6-dione

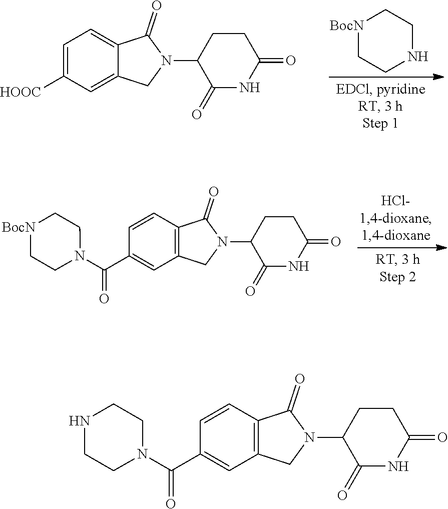 [SMILES: O=C1CCC(N2Cc3cc(C(=O)O)ccc3C2=O)C(=O)N1 ; CC(C)(C)OC(=O)N1CCNCC1 ; CC(C)(C)OC(=O)N1CCN(C(=O)c2ccc3c(c2)CN(C2CCC(=O)NC2=O)C3=O)CC1 ; O=C1CCC(N2Cc3cc(C(=O)N4CCNCC4)ccc3C2=O)C(=O)N1]

Step 1. Preparation of tert-butyl 4-(2-(2,6-dioxopiperidin-3-yl)-1-oxoisoindoline-5-carbonyl)piperazine-1-carboxylate. To a stirred mixture of 2-(2,6-dioxopiperidin-3-yl)-1-oxoisoindole-5-carboxylic acid (1.5 g, 5.204 mmol, 1 equiv) and tert-butyl piperazine-1-carboxylate (1.34 g, 15.612 mmol, 3 equiv) in pyridine (20 ml) was added EDCI (1.50 g, 7.806 mmol, 1.5 equiv) in portions at room temperature under nitrogen atmosphere. The resulting mixture was stirred for additional 3 h at room temperature. The resulting mixture was extracted with CHCl₃ (3×10 mL). The combined organic layers were washed with NaCl (aq) (3×10 mL), dried over anhydrous Na₂SO₄. After filtration, the filtrate was concentrated under reduced pressure. The resulting mixture was concentrated under vacuum. The residue was purified by reverse-phase flash chromatography to afford tert-butyl 4-(2-(2,6-dioxopiperidin-3-yl)-1-oxoisoindoline-5-carbonyl)piperazine-1-carboxylate (1.2 g, 65%) as an off-white solid.

Step 2. Preparation of 3-(1-oxo-5-(piperazine-1-carbonyl)isoindolin-2-yl)piperidine-2,6-dione. To a stirred solution of tert-butyl 4-(2-(2,6-dioxopiperidin-3-yl)-1-oxoisoindoline-5-carbonyl)piperazine-1-carboxylate (1.2 g, 2.629 mmol, 1 equiv) in 1,4-dioxane (10 mL) was added HCl in 1,4-dioxane (15 mL, 15.0 mmol, 5.71 equiv) in portions at 0° C. under nitrogen atmosphere. The resulting mixture was stirred for additional 3 h at room temperature. The resulting mixture was concentrated under vacuum. The residue was purified by reverse-phase flash chromatography to afford 3-(1-oxo-5-(piperazine-1-carbonyl)isoindolin-2-yl)piperidine-2,6-dione (62 mg, 6%) as an off-white solid.

LC-MS (ES, m/z)=357.15 [M+1]⁺. ¹H NMR (400 MHz, DMSO-d₆) δ 11.02 (s, 1H), 9.07 (s, 2H), 7.82 (d, J=7.8 Hz, 1H), 7.71 (s, 1H), 7.59 (d, J=7.8 Hz, 1H), 5.15 (dd, J=13.3, 5.1 Hz, 1H), 4.73 (s, 1H), 4.52 (d, J=17.6 Hz, 1H), 3.83 (s, 2H), 3.56 (S, 2H), 3.18 (s, 4H), 2.99-2.86 (m, 1H), 2.67-2.57 (m, 1H), 2.42 (qd, J=13.2, 4.4 Hz, 1H), 2.10-1.97 (m, 1H).

### Example 19

### Synthesis of 3-(5-(morpholine-4-carbonyl)-1-oxoisoindolin-2-yl)piperidine-2,6-dione

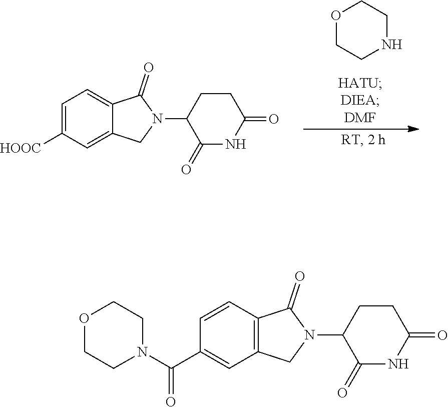 [SMILES: C1COCCN1 ; O=C1CCC(N2Cc3cc(C(=O)O)ccc3C2=O)C(=O)N1 ; O=C1CCC(N2Cc3cc(C(=O)N4CCOCC4)ccc3C2=O)C(=O)N1]

Step 1. Preparation of 3-(5-(morpholine-4-carbonyl)-1-oxoisoindolin-2-yl)piperidine-2,6-dione. To a stirred solution of 2-(2,6-dioxopiperidin-3-yl)-1-oxoisoindole-5-carboxylic acid (100 mg, 0.347 mmol, 1 equiv) and morpholine (36.3 mg, 0.416 mmol, 1.2 equiv) in DMF (2 mL, 25.843 mmol, 74.50 equiv) was added DIEA (134.5 mg, 1.041 mmol, 3 equiv) at room temperature. The resulting mixture was stirred for 10 min at room temperature. To the above mixture was added HATU (197.9 mg, 0.520 mmol, 1.5 equiv) at room temperature. The resulting mixture was stirred for additional 2 h at room temperature. The residue was purified by reverse-phase flash chromatography to afford 3-(5-(morpholine-4-carbonyl)-1-oxoisoindolin-2-yl)piperidine-2,6-dione (53 mg, 43%) as a white solid.

LC-MS (ES, m/z)=358 [M+1]⁺. ¹H NMR (400 MHz, DMSO-d₆) δ 10.99 (s, 1H), 7.79 (d, J=7.7 Hz, 1H), 7.66 (s, 1H), 7.53 (dd, J=7.7, 1.3 Hz, 1H), 5.14 (dd, J=13.3, 5.1 Hz, 1H), 4.51 (d, J=17.5 Hz, 1H), 4.38 (d, J=17.5 Hz, 1H), 3.65 (s, 7H), 2.92 (ddd, J=17.3, 13.6, 5.4 Hz, 1H), 2.61 (ddd, J=17.3, 4.4, 2.3 Hz, 1H), 2.41 (qd, J=13.2, 4.4 Hz, 1H), 2.01 (dtd, J=12.7, 5.4, 2.3 Hz, 1H).

### Example 20

### Synthesis of 3-(1-oxo-5-(7-(trifluoromethyl)indoline-1-carbonyl)isoindolin-2-yl)piperidine-2,6-dione

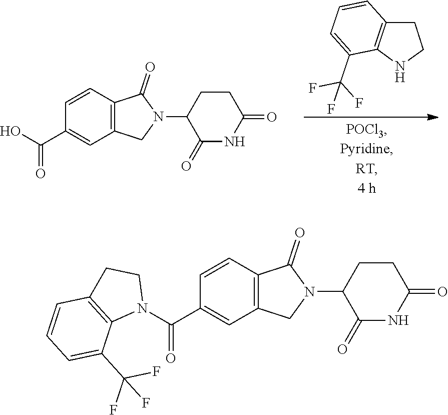 [SMILES: O=C1CCC(N2Cc3cc(C(=O)O)ccc3C2=O)C(=O)N1 ; FC(F)(F)c1cccc2c1NCC2 ; O=C1CCC(N2Cc3cc(C(=O)N4CCc5cccc(C(F)(F)F)c54)ccc3C2=O)C(=O)N1]

Step 1. Preparation of 3-(1-oxo-5-(7-(trifluoromethyl)indoline-1-carbonyl)isoindolin-2-yl)piperidine-2,6-dione. To a stirred mixture of 2-(2,6-dioxopiperidin-3-yl)-1-oxoisoindole-5-carboxylic acid (50 mg, 0.173 mmol, 1 equiv) and 7-(trifluoromethyl)-2,3-dihydro-1H-indole (32.5 mg, 0.173 mmol, 1 equiv) in pyridine (1 mL) was added POCl₃ (79.78 mg, 0.519 mmol, 3 equiv) at 0° C. under nitrogen atmosphere. The resulting mixture was stirred for 4 h at room temperature. The reaction was quenched by the addition of sat. NH₄Cl (aq.) (1 mL) at 0° C. The resulting mixture was extracted with EtOAc (3×2 mL). The combined organic layers were washed with brine (2×1 mL), dried over anhydrous Na₂SO₄. After filtration, the filtrate was concentrated under reduced pressure. The residue was purified by reverse-phase flash chromatography to afford 3-(1-oxo-5-(7-(trifluoromethyl)indoline-1-carbonyl)isoindolin-2-yl)piperidine-2,6-dione (5.4 mg, 7%) as a white solid.

LC-MS (ES, m/z)=458.15 [M+1]⁺. ¹H NMR (400 MHz, DMSO-d₆) δ 11.03 (s, 1H), 7.98 (s, 1H), 7.91-7.82 (m, 2H), 7.62 (d, J=7.5 Hz, 1H), 7.55 (d, J=8.0 Hz, 1H), 7.35 (t, J=7.6 Hz, 1H), 5.17 (dd, J=13.3, 5.1 Hz, 1H), 4.56 (d, J=17.7 Hz, 1H), 4.43 (d, J=17.7 Hz, 1H), 4.10 (t, J=7.6 Hz, 2H), 3.07 (t, J=7.6 Hz, 2H), 2.97-2.87 (m, 1H), 2.62 (d, J=17.1 Hz, 1H), 2.43 (dd, J=13.2, 4.5 Hz, 1H), 2.05 (dd, J=11.8, 5.7 Hz, 1H), 1.24 (s, 1H).

### Example 21

### Synthesis of 3-(1-oxo-5-(4-(trifluoromethyl)isoindoline-2-carbonyl)isoindolin-2-yl)piperidine-2,6-dione

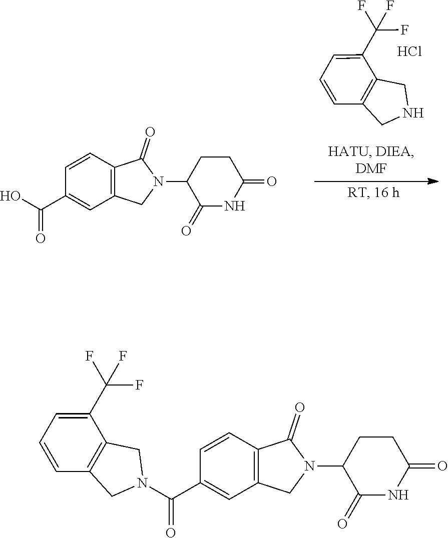 [SMILES: FC(F)(F)c1cccc2c1CNC2 ; Cl ; O=C1CCC(N2Cc3cc(C(=O)O)ccc3C2=O)C(=O)N1 ; O=C1CCC(N2Cc3cc(C(=O)N4Cc5cccc(C(F)(F)F)c5C4)ccc3C2=O)C(=O)N1]

Step 1. Preparation of 3-(1-oxo-5-(4-(trifluoromethyl)isoindoline-2-carbonyl)isoindolin-2-yl)piperidine-2,6 dione. To a stirred mixture of 2-(2,6-dioxopiperidin-3-yl)-1-oxoisoindole-5-carboxylic acid (150 mg, 0.520 mmol, 1.00 equiv), 2-(2,6-dioxopiperidin-3-yl)-1-oxo-3H-isoindole-5-carboxylic acid (150 mg, 0.520 mmol, 1.00 equiv) and DIEA (201.8 mg, 1.560 mmol, 3 equiv) in DMF (3 mL) was added HATU (296.8 mg, 0.780 mmol, 1.5 equiv) in portions at room temperature under nitrogen atmosphere. The resulting mixture was stirred for overnight at room temperature under nitrogen atmosphere. The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product was purified by Prep-HPLC to afford 3-(1-oxo-5-(4-(trifluoromethyl)isoindoline-2-carbonyl)isoindolin-2-yl)piperidine-2,6-dione (91 mg, 37%) as an off-white solid. LC-MS (ES, m/z)=458.05 [M+1]⁺. ¹H NMR (400 MHz, DMSO-d₆) δ 11.02 (s, 1H), 7.89-7.81 (m, 2H), 7.75 (td, J=8.6, 7.7, 1.4 Hz, 1H), 7.67 (dd, J=11.2, 7.6 Hz, 1H), 7.56 (dt, J=15.8, 7.6 Hz, 2H), 5.25-5.12 (m, 1H), 5.03 (s, 1H), 4.97 (s, 1H), 4.92 (s, 1H), 4.84 (s, 1H), 4.55 (dd, J=17.5, 3.6 Hz, 1H), 4.41 (d, J=17.5 Hz, 1H), 2.93 (ddd, J=17.2, 13.6, 5.4 Hz, 1H), 2.62 (ddd, J=17.3, 4.5, 2.3 Hz, 1H), 2.48-2.33 (m, 1H), 2.04 (dtd, J=10.2, 5.1, 2.7 Hz, 1H).

### Example 22

### Synthesis of 3-(1-oxo-5-(5-(trifluoromethyl)isoindoline-2-carbonyl)isoindolin-2-yl)piperidine-2,6-dione

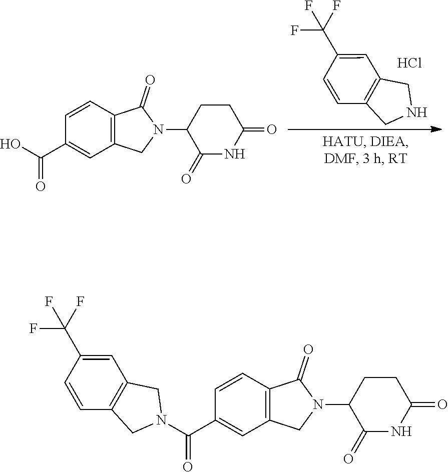 [SMILES: O=C1CCC(N2Cc3cc(C(=O)O)ccc3C2=O)C(=O)N1 ; FC(F)(F)c1ccc2c(c1)CNC2 ; Cl ; O=C1CCC(N2Cc3cc(C(=O)N4Cc5ccc(C(F)(F)F)cc5C4)ccc3C2=O)C(=O)N1]

Step 1. Preparation of 3-(1-oxo-5-(5-(trifluoromethyl)isoindoline-2-carbonyl)isoindolin-2-yl)piperidine-2,6-dione. A solution of 5-(trifluoromethyl)-2,3-dihydro-1H-isoindole (97.4 mg, 0.520 mmol, 1.5 equiv), 2-(2,6-dioxopiperidin-3-yl)-1-oxoisoindole-5-carboxylic acid (100 mg, 0.347 mmol, 1.00 equiv) and DIEA (134.5 mg, 1.041 mmol, 3 equiv) in DMF (3 mL) was stirred for 5 min at room temperature. To the above mixture was added HATU (197.9 mg, 0.520 mmol, 1.5 equiv) dropwise over 5 min at room temperature. The resulting mixture was stirred for additional 3 h at room temperature. The reaction was quenched by the addition of Water (1 mL) at 0° C. The resulting mixture was concentrated under reduced pressure. The residue was purified by reverse-phase flash chromatography to afford 3-(1-oxo-5-(5-(trifluoromethyl)isoindoline-2-carbonyl)isoindolin-2-yl)piperidine-2,6-dione (54 mg, 34%) as an off-white solid.

LC-MS (ES, m/z)=456 [M+1]⁺. ¹H NMR (400 MHz, DMSO-d₆) δ 11.02 (s, 1H), 7.85 (d, J=4.7 Hz, 1H), 7.82 (p, J=2.4 Hz, 1H), 7.73 (dt, J=7.8, 1.9 Hz, 1H), 7.69-7.62 (m, 2H), 7.49 (d, J=8.0 Hz, 1H), 5.16 (dd, J=13.3, 5.0 Hz, 1H), 4.97 (s, 1H), 4.88-4.69 (m, 3H), 4.58-4.48 (m, 1H), 4.40 (d, J=17.6 Hz, 1H), 2.93 (ddd, J=17.4, 13.6, 5.4 Hz, 1H), 2.66-2.57 (m, 1H), 2.42 (td, J=13.2, 4.4 Hz, 1H), 2.08-2.00 (m, 1H), 1.24 (s, 1H).

### Example 23

### Synthesis of 3-(1-oxo-5-(1,2,3,4-tetrahydroisoquinoline-2-carbonyl)isoindolin-2-yl)piperidine-2,6-dione

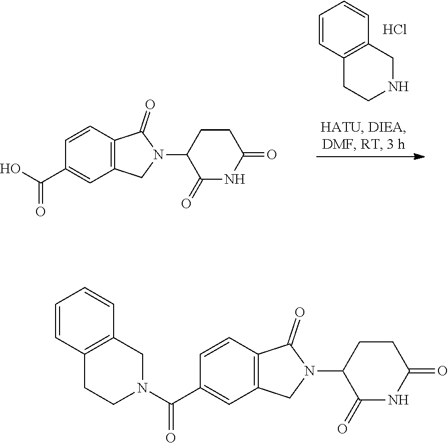 [SMILES: c1ccc2c(c1)CCNC2 ; Cl ; O=C1CCC(N2Cc3cc(C(=O)O)ccc3C2=O)C(=O)N1 ; O=C1CCC(N2Cc3cc(C(=O)N4CCc5ccccc5C4)ccc3C2=O)C(=O)N1]

Step 1. Preparation of 3-(1-oxo-5-(1,2,3,4-tetrahydroisoquinoline-2-carbonyl)isoindolin-2-yl)piperidine-2,6-dione. To a stirred mixture of tetrahydroisoquinoline hydrochloride (200 mg, 1.179 mmol, 1 equiv), 2-(2,6-dioxopiperidin-3-yl)-1-oxoisoindole-5-carboxylic acid (373.81 mg, 1.297 mmol, 1.1 equiv) and DIEA (761.84 mg, 5.895 mmol, 5 equiv) in DMF (2 mL) was added HATU (672.39 mg, 1.768 mmol, 1.5 equiv) in portions at room temperature under nitrogen atmosphere. The resulting mixture was stirred for 3 h at room temperature. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(1-oxo-5-(1,2,3,4-tetrahydroisoquinoline-2-carbonyl)isoindolin-2-yl)piperidine-2,6-dione (153 mg, 32%) as an off-white solid.

LC-MS (ES, m/z)=404.05 [M+1]⁺. ¹H NMR (400 MHz, DMSO-d₆) δ 11.03 (s, 1H), 7.81 (d, J=7.8 Hz, 1H), 7.73-7.52 (m, 2H), 7.23 (d, J=33.1 Hz, 4H), 5.15 (dd, J=13.3, 5.1 Hz, 1H), 4.80 (s, 1H), 4.45 (dd, J=52.4, 17.7 Hz, 3H), 3.88 (s, 1H), 3.55 (s, 1H), 2.90 (dt, J=20.7, 14.4 Hz, 3H), 2.65-2.56 (m, 1H), 2.42 (qd, J=13.2, 4.4 Hz, 1H), 2.06-1.99 (m, 1H).

### Example 24

### Synthesis of 3-(1-oxo-5-(1,2,3,4-tetrahydroquinoline-1-carbonyl)isoindolin-2-yl)piperidine-2,6-dione

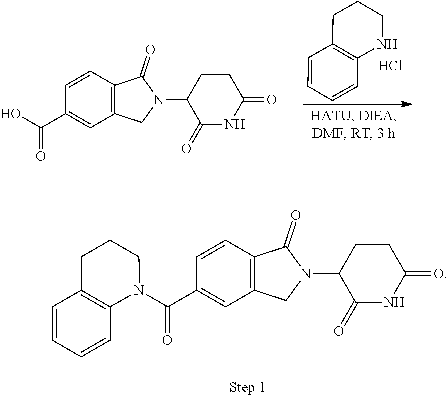

Step 1. Preparation of 3-(1-oxo-5-(1,2,3,4-tetrahydroquinoline-1-carbonyl)isoindolin-2-yl)piperidine-2,6-dione. To a stirred mixture of tetrahydroquinoline hydrochloride (200 mg, 1.179 mmol, 1 equiv), 2-(2,6-dioxopiperidin-3-yl)-1-oxoisoindole-5-carboxylic acid (373.8 mg, 1.297 mmol, 1.1 equiv) and DIEA (761.8 mg, 5.895 mmol, 5 equiv) in DMF (2 mL) was added HATU (672.4 mg, 1.768 mmol, 1.5 equiv) in portions at room temperature under nitrogen atmosphere. The resulting mixture was stirred for 3 h at room temperature. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The crude product was purified by Prep-HPLC to afford 3-(1-oxo-5-(1,2,3,4-tetrahydroquinoline-1-carbonyl)isoindolin-2-yl)piperidine-2,6-dione (24 mg, 5%) as an off-white solid.

LC-MS (ES, m/z)=404 [M+1]⁺. ¹H NMR (400 MHz, DMSO-d₆) δ 11.00 (s, 1H), 7.70-7.62 (m, 2H), 7.38 (d, J=7.8 Hz, 1H), 7.21 (dd, J=7.5, 1.5 Hz, 1H), 7.01 (td, J=7.4, 1.3 Hz, 1H), 6.92 (t, J=7.6 Hz, 1H), 6.83 (s, 1H), 5.11 (dd, J=13.3, 5.1 Hz, 1H), 4.45 (d, J=17.6 Hz, 1H), 4.32 (d, J=17.6 Hz, 1H), 3.77 (td, J=6.3, 2.8 Hz, 2H), 3.00-2.78 (m, 3H), 2.60 (ddd, J=17.2, 4.4, 2.2 Hz, 1H), 2.39 (qd, J=13.2, 4.4 Hz, 1H), 1.98 (dp, J=13.1, 7.1, 6.6 Hz, 3H).

### Example 25

### Synthesis of (2-(2,6-dioxopiperidin-3-yl)-1-oxoisoindoline-5-carbonyl)-L-proline

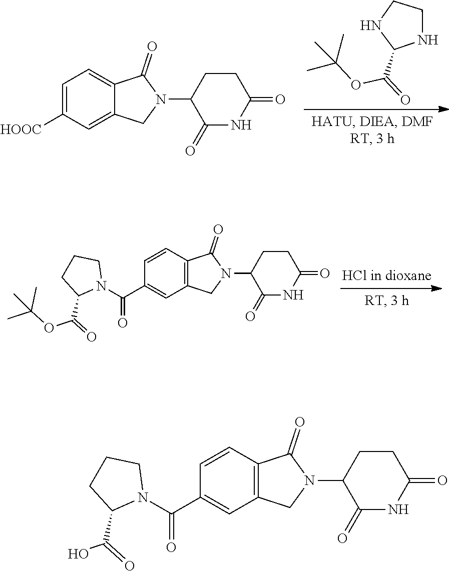

Step 1. Preparation of tert-butyl (2-(2,6-dioxopiperidin-3-yl)-1-oxoisoindoline-5-carbonyl)-L-prolinate. To a stirred mixture of 2-(2,6-dioxopiperidin-3-yl)-1-oxoisoindole-5-carboxylic acid (300 mg, 1.041 mmol, 1 equiv) and tert-butyl (2R)-pyrrolidine-2-carboxylate hydrochloride (36.0 mg, 0.173 mmol, 1 equiv) in DMF (6 ml) were added DIEA (0.54 mL, 3.123 mmol, 3 equiv) and HATU (593.6 mg, 1.561 mmol, 1.5 equiv) at room temperature under nitrogen atmosphere. The resulting mixture was stirred for additional 3 h at room temperature. The resulting mixture was extracted with EtOAc (3×10 mL). The combined organic layers were washed with NaCl(aq) (3×3 mL), dried over anhydrous Na₂SO₄. After filtration, the filtrate was concentrated under reduced pressure. The resulting mixture was concentrated under reduced pressure. The residue was purified by reverse-phase flash chromatography to afford tert-butyl (2-(2,6-dioxopiperidin-3-yl)-1-oxoisoindoline-5-carbonyl)-L-prolinate (280 mg, 61%) as a yellow solid.

Step 2. Preparation of (2-(2,6-dioxopiperidin-3-yl)-1-oxoisoindoline-5-carbonyl)-L-proline. To a stirred solution of tert-butyl (2-(2,6-dioxopiperidin-3-yl)-1-oxoisoindoline-5-carbonyl)-L-prolinate (220 mg, 0.498 mmol, 1 equiv) in DCM (5 mL) was added TFA (1 mL) in portions at 0° C. The resulting mixture was stirred for additional 3 h at room temperature. The resulting mixture was concentrated under vacuum. The crude product was purified by Prep-HPLC to afford (2-(2,6-dioxopiperidin-3-yl)-1-oxoisoindoline-5-carbonyl)-L-proline (89 mg, 46%) as an off-white solid.

LC-MS (ES, m/z)=386.15 [M+1]⁺. ¹H NMR (400 MHz, DMSO-d₆) δ 12.59 (s, 1H), 11.02 (s, 1H), 7.80 (d, J=7.8 Hz, 1H), 7.74 (d, J=7.1 Hz, 2H), 5.14 (dd, J=13.3, 5.0 Hz, 1H), 4.50 (td, J=16.6, 15.7, 6.7 Hz, 3H), 3.60 (t, J=7.0 Hz, 2H), 3.53-3.42 (m, 1H), 2.99-2.85 (m, 1H), 2.65-2.56 (m, 1H), 2.41 (qd, J=13.1, 4.3 Hz, 1H), 2.33-2.22 (m, 1H), 1.88 (dtd, J=20.8, 11.9, 11.3, 6.0 Hz, 3H).

### Example 26

### Synthesis of (2-(2,6-dioxopiperidin-3-yl)-1-oxoisoindoline-5-carbonyl)-D-proline

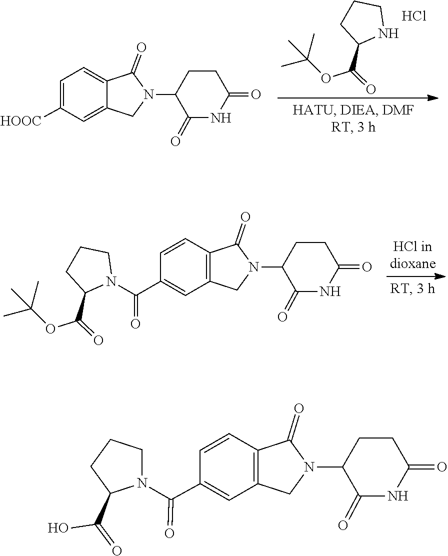

Step 1. Preparation of tert-butyl (2-(2,6-dioxopiperidin-3-yl)-1-oxoisoindoline-5-carbonyl)-D-prolinate. To a stirred mixture of 2-(2,6-dioxopiperidin-3-yl)-1-oxoisoindole-5-carboxylic acid (300 mg, 1.041 mmol, 1 equiv) and tert-butyl (2R)-pyrrolidine-2-carboxylate hydrochloride (39.6 mg, 0.190 mmol, 1.1 equiv) in DMF (6 ml) were added DIEA (0.73 mL, 4.164 mmol, 4 equiv) and HATU (594 mg, 1.561 mmol, 1.5 equiv) in portions at room temperature under nitrogen atmosphere. The resulting mixture was stirred for additional 3 h at room temperature. The resulting mixture was extracted with EtOAc (3×10 mL). The combined organic layers were washed with NaCl(aq) (3×3 mL), dried over anhydrous Na₂SO₄. After filtration, the filtrate was concentrated under reduced pressure. The resulting mixture was concentrated under reduced pressure. The residue was purified by reverse-phase flash chromatography to afford tert-butyl (2-(2,6-dioxopiperidin-3-yl)-1-oxoisoindoline-5-carbonyl)-D-prolinate (105 mg, 23%) as an off-white solid.

Step 2. Preparation of (2-(2,6-dioxopiperidin-3-yl)-1-oxoisoindoline-5-carbonyl)-D-proline. To a stirred solution of tert-butyl (2-(2,6-dioxopiperidin-3-yl)-1-oxoisoindoline-5-carbonyl)-D-prolinate (220 mg, 0.498 mmol, 1 equiv) in DCM (5 mL) was added TFA (1 mL) in portions at 0° C. under air atmosphere. The resulting mixture was stirred for additional 3 h at room temperature. The resulting mixture was concentrated under vacuum. The crude product was purified by Prep-HPLC to afford (2-(2,6-dioxopiperidin-3-yl)-1-oxoisoindoline-5-carbonyl)-D-proline (89 mg, 46%) as an off-white solid.

LC-MS (ES, m/z)=386.15 [M+1]⁺. ¹H NMR (400 MHz, DMSO-d₆) δ 12.59 (s, 1H), 11.02 (s, 1H), 7.80 (d, J=7.8 Hz, 1H), 7.62 (dd, J=6.4, 2.4 Hz, 2H), 5.14 (dd, J=13.3, 5.0 Hz, 1H), 4.50 (td, J=16.6, 15.7, 6.7 Hz, 3H), 3.60 (t, J=7.0 Hz, 2H), 2.99-2.85 (m, 1H), 2.65-2.56 (m, 1H), 2.41 (qd, J=13.1, 4.3 Hz, 1H), 2.33-2.22 (m, 1H), 2.03 (dtd, J=12.5, 5.2, 2.3 Hz, 1H), 1.88 (dtd, J=20.8, 11.9, 11.3, 6.0 Hz, 3H).

### Example 27

### Synthesis of 3-(1-oxo-5-(2-oxopiperidine-1-carbonyl)isoindolin-2-yl)piperidine-2,6-dione

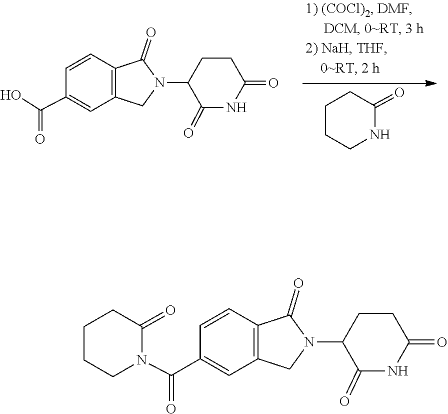 [SMILES: O=C1CCC(N2Cc3cc(C(=O)O)ccc3C2=O)C(=O)N1 ; O=C1CCCCN1 ; O=C1CCC(N2Cc3cc(C(=O)N4CCCCC4=O)ccc3C2=O)C(=O)N1]

Step 1. Preparation of 3-(1-oxo-5-(2-oxopiperidine-1-carbonyl)isoindolin-2-yl)piperidine-2,6-dione. A solution of 2-(2,6-dioxopiperidin-3-yl)-1-oxo-3H-isoindole-5-carboxylic acid (200 mg, 0.694 mmol, 1 equiv) in DCM (3 mL) was treated with DMF (0.1 mL) for 0.5 h at 0° C. under nitrogen atmosphere followed by the addition of (COCl)₂ (440.30 mg, 3.470 mmol, 5 equiv) dropwise at 0° C. The resulting mixture was stirred for 3 h at room temperature under nitrogen atmosphere to obtain solution A. A solution of 2-piperidinone (343.90 mg, 3.470 mmol, 5 equiv) in THF (4 mL) was treated with NaH (66.60 mg, 2.776 mmol, 4 equiv) for 0.5 h at 0° C. under nitrogen atmosphere to obtain solution B. Solution A and solution B were mixed and stirred for 2 h at room temperature under nitrogen atmosphere. The reaction was quenched by the addition of sat. NH₄Cl (aq.) (5 mL) at 0° C. The resulting mixture was concentrated under reduced pressure. The crude product was purified by Prep-HPLC to afford 3-(1-oxo-5-(2-oxopiperidine-1-carbonyl)isoindolin-2-yl)piperidine-2,6-dione (3 mg, 1.2%) as an off-white solid.

LC-MS (ES, m/z)=370 [M+1]⁺. ¹H NMR (400 MHz, DMSO-d₆) δ 10.71 (s, 1H), 7.77-7.70 (m, 2H), 7.66-7.59 (m, 1H), 5.14 (dd, J=13.3, 5.2 Hz, 1H), 4.50 (d, J=17.5 Hz, 1H), 4.37 (d, J=17.5 Hz, 1H), 3.73 (t, J=5.7 Hz, 2H), 2.92 (ddd, J=17.1, 13.5, 5.3 Hz, 1H), 2.66-2.52 (m, 2H), 2.41 (qd, J=13.1, 4.5 Hz, 2H), 2.10-1.98 (m, 2H), 1.98-1.81 (m, 4H), 1.24 (s, 1H).

### Example 28

### Synthesis of 3-(5-(2,3-dihydro-1H-pyrrolo[3,2-b]pyridine-1-carbonyl)-1-oxoisoindolin-2-yl)piperidine-2,6-dione

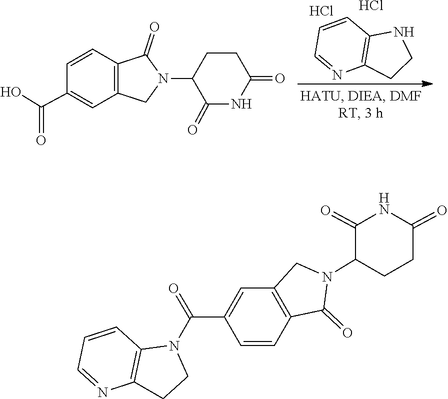

Step 1. Preparation of 3-(5-(2,3-dihydro-1H-pyrrolo[3,2-b]pyridine-1-carbonyl)-1-oxoisoindolin-2-yl)piperidine-2,6-dione. A solution of 1H,2H, 3H-pyrrolo[3,2-b]pyridine dihydrochloride (200 mg, 1.036 mmol, 1.00 equiv) and DIEA (201 mg, 1.554 mmol, 1.5 equiv) in DMF (2.5 mL) was stirred for 5 min at room temperature under air atmosphere. To the above mixture was added 2-(2,6-dioxopiperidin-3-yl)-1-oxoisoindole-5-carboxylic acid (358.3 mg, 1.243 mmol, 1.2 equiv) and HATU (590.8 mg, 1.554 mmol, 1.5 equiv) in portions 5 min at room temperature. The resulting mixture was filtered. The resulting mixture was filtered, the filter cake was washed with acetonitrile (3×7 mL). The filtrate was concentrated under reduced pressure. The resulting mixture was concentrated under reduced pressure. The filtrate (5 mL) was purified by Prep-HPLC to afford 3-(5-(2,3-dihydro-1H-pyrrolo[3,2-b]pyridine-1-carbonyl)-1-oxoisoindolin-2-yl)piperidine-2,6-dione (9.8 mg, 2.11%) as an off-white solid.

LC-MS (ES, m/z)=391 [M+1]⁺. ¹H NMR (400 MHz, DMSO-d₆) δ 11.02 (s, 1H), 8.31 (s, 1H), 8.18 (d, J=4.6 Hz, 1H), 7.88-7.82 (m, 2H), 7.74 (d, J=7.9 Hz, 1H), 7.22 (s, 1H), 5.16 (dd, J=13.3, 5.1 Hz, 1H), 4.54 (d, J=17.6 Hz, 1H), 4.41 (d, J=17.6 Hz, 1H), 4.04 (d, J=8.7 Hz, 3H), 3.19 (t, J=8.4 Hz, 2H), 2.93 (ddd, J=17.2, 13.6, 5.4 Hz, 1H), 2.70-2.54 (m, 2H), 2.41 (td, J=13.2, 4.5 Hz, 1H), 2.03 (ddd, J=10.5, 5.3, 2.8 Hz, 1H).

### Example 29

### Synthesis of 3-(5-(2,3-dihydro-1H-pyrrolo[3,2-c]pyridine-1-carbonyl)-1-oxoisoindolin-2-yl)piperidine-2,6-dione

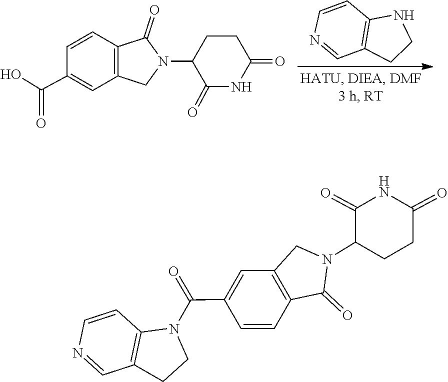

Step 1. Preparation of 3-(5-(2,3-dihydro-1H-pyrrolo[3,2-c]pyridine-1-carbonyl)-1-oxoisoindolin-2-yl)piperidine-2,6-dione. A solution of 1H,2H,3H-pyrrolo[3,2-c]pyridine (100 mg, 0.832 mmol, 1 equiv), DIEA (161.4 mg, 1.248 mmol, 1.5 equiv) and 2-(2,6-dioxopiperidin-3-yl)-1-oxo-3H-isoindole-5-carboxylic acid (287.9 mg, 0.998 mmol, 1.2 equiv) in DMF (3 mL) was stirred for 5 min at room temperature. To the above mixture was added HATU (474.7 mg, 1.248 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The crude product was purified by Prep-HPLC to afford 3-(5-(2,3-dihydro-1H-pyrrolo[3,2-c]pyridine-1-carbonyl)-1-oxoisoindolin-2-yl)piperidine-2,6-dione (10 mg, 3.1%) as an off-white solid.

LC-MS (ES, m/z)=391 [M+1]⁺. ¹H NMR (400 MHz, DMSO-d₆) δ 11.04 (s, 1H), 8.67 (s, 1H), 8.62 (d, J=6.5 Hz, 1H), 7.95 (d, J=8.1 Hz, 2H), 7.90 (d, J=7.8 Hz, 1H), 7.81 (dd, J=7.8, 1.4 Hz, 1H), 5.17 (dd, J=13.3, 5.1 Hz, 1H), 4.56 (d, J=17.7 Hz, 1H), 4.44 (d, J=17.7 Hz, 1H), 4.21 (t, J=8.6 Hz, 2H), 3.28 (t, J=8.6 Hz, 2H), 2.93 (ddd, J=17.3, 13.6, 5.4 Hz, 1H), 2.66-2.58 (m, 1H), 2.50-2.36 (m, 1H), 2.04 (dtd, J=10.8, 5.8, 5.4, 2.9 Hz, 1H).

### Example 30

### Synthesis of 3-(5-(6,7-dihydro-5H-pyrrolo[3,4-b]pyridine-6-carbonyl)-1-oxoisoindolin-2-yl)piperidine-2,6-dione

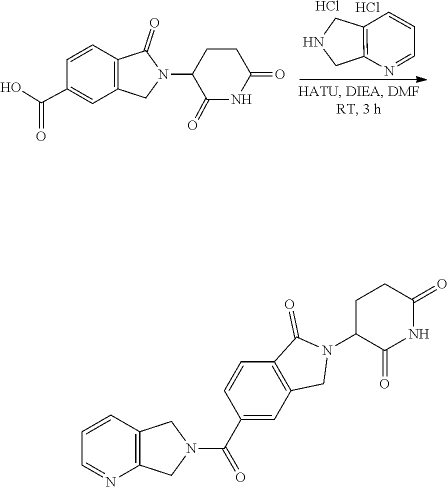 [SMILES: Cl ; Cl ; O=C1CCC(N2Cc3cc(C(=O)O)ccc3C2=O)C(=O)N1 ; c1cnc2c(c1)CNC2 ; O=C1CCC(N2Cc3cc(C(=O)N4Cc5cccnc5C4)ccc3C2=O)C(=O)N1]

Step 1. Preparation of 3-(5-(6,7-dihydro-5H-pyrrolo[3,4-b]pyridine-6-carbonyl)-1-oxoisoindolin-2-yl)piperidine-2,6-dione. A solution of 5H,6H,7H-pyrrolo[3,4-b]pyridine dihydrochloride (200 mg, 1.036 mmol, 1.00 equiv) and DIEA (200.8 mg, 1.554 mmol, 1.5 equiv) in DMF (5 mL) was stirred for 5 min at room temperature under air atmosphere. To the above mixture was added 2-(2,6-dioxopiperidin-3-yl)-1-oxoisoindole-5-carboxylic acid (358 mg, 1.243 mmol, 1.2 equiv), HATU (591 mg, 1.554 mmol, 1.5 equiv) in portions 5 min at room temperature. The resulting mixture was stirred for additional 3 h at room temperature. The resulting mixture was filtered, the filter cake was washed with acetonitrile (3×7 mL). The filtrate was concentrated under reduced pressure. The resulting mixture was concentrated under reduced pressure. The filtrate (5 mL) was purified by Prep-HPLC to afford 3-(5-(6,7-dihydro-5H-pyrrolo[3,4-b]pyridine-6-carbonyl)-1-oxoisoindolin-2-yl)piperidine-2,6-dione (24 mg, 6%) as an off-white solid.

LC-MS (ES, m/z)=391 [M+1]⁺. ¹H NMR (400 MHz, DMSO-d₆) δ 11.01 (s, 1H), 8.50-8.42 (m, 1H), 7.89-7.80 (m, 2H), 7.78-7.69 (m, 1H), 7.30 (s, 1H), 5.16 (dd, J=13.0, 4.9 Hz, 1H), 4.93 (s, 1H), 4.86 (s, 1H), 4.79 (d, J=6.9 Hz, 2H), 4.54 (d, J=17.4 Hz, 1H), 4.46-4.36 (m, 1H), 2.91 (d, J=12.3 Hz, 2H), 2.63 (s, 2H), 2.58 (s, 1H), 2.03 (s, 1H), −0.13 (s, 2H).

### Example 31

### Synthesis of 3-(5-(2,3-dihydro-1H-pyrrolo[3,4-c]pyridine-2-carbonyl)-1-oxoisoindolin-2-yl)piperidine-2,6-dione

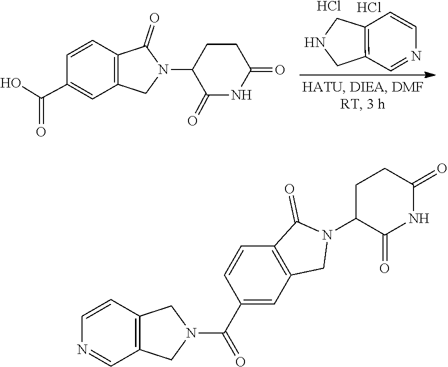 [SMILES: Cl ; Cl ; O=C1CCC(N2Cc3cc(C(=O)O)ccc3C2=O)C(=O)N1 ; c1cc2c(cn1)CNC2 ; O=C1CCC(N2Cc3cc(C(=O)N4Cc5ccncc5C4)ccc3C2=O)C(=O)N1]

Step 1. Preparation of 3-(5-(2,3-dihydro-1H-pyrrolo[3,4-c]pyridine-2-carbonyl)-1-oxoisoindolin-2-yl)piperidine-2,6-dione. A solution of 1H,2H, 3H-pyrrolo[3,4-c]pyridine dihydrochloride (200 mg, 1.665 mmol, 1 equiv) and DIEA (322.7 mg, 2.498 mmol, 1.5 equiv) in DMF (3.5 mL) was stirred for 5 min at room temperature. To the above mixture was added 2-(2,6-dioxopiperidin-3-yl)-1-oxoisoindole-5-carboxylic acid (576 mg, 1.998 mmol, 1.2 equiv) and HATU (591 mg, 1.554 mmol, 1.5 equiv) in portions 5 min at room temperature. The resulting mixture was stirred for additional 3 h at room temperature. The resulting mixture was filtered, the filter cake was washed with acetonitrile (3×7 mL). The filtrate was concentrated under reduced pressure. The resulting mixture was concentrated under reduced pressure. The filtrate (5 mL) was purified by Prep-HPLC to afford 3-(5-(2,3-dihydro-1H-pyrrolo[3,4-c]pyridine-2-carbonyl)-1-oxoisoindolin-2-yl)piperidine-2,6-dione (8.1 mg, 2%) as an off-white solid.

LC-MS (ES, m/z)=391 [M+1]⁺. ¹H NMR (400 MHz, DMSO-d₆) δ 11.03 (s, 1H), 8.90-8.62 (m, 2H), 7.89-7.79 (m, 3H), 7.74 (d, J=7.9 Hz, 1H), 7.67 (d, J=5.5 Hz, 1H), 5.17 (dd, J=13.3, 5.1 Hz, 1H), 5.04 (d, J=8.2 Hz, 2H), 4.96-4.87 (m, 2H), 4.56 (s, 1H), 4.52 (s, 1H), 4.41 (d, J=17.6 Hz, 2H), 2.94 (ddd, J=17.2, 13.6, 5.4 Hz, 1H), 2.62 (dt, J=17.3, 3.6 Hz, 1H), 2.43 (tt, J=13.2, 6.5 Hz, 1H), 2.05 (qd, J=6.7, 6.0, 3.4 Hz, 1H).

### Example 32

### Synthesis of 3-(5-(6-fluoroindoline-1-carbonyl)-1-oxoisoindolin-2-yl)piperidine-2,6-dione

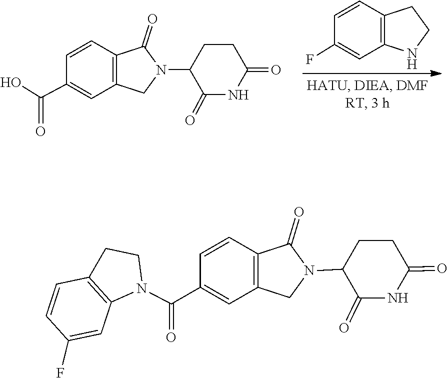

Step 1. Preparation of 3-(5-(6-fluoroindoline-1-carbonyl)-1-oxoisoindolin-2-yl)piperidine-2,6-dione. To a stirred solution of 2-(2,6-dioxopiperidin-3-yl)-1-oxoisoindole-5-carboxylic acid (200 mg, 0.694 mmol, 1 equiv), 6-fluoro-2,3-dihydro-1H-indole (95.2 mg, 0.694 mmol, 1 equiv) and DIEA (448.4 mg, 3.470 mmol, 5 equiv) in DMF (2 ml) were added HATU (395.7 mg, 1.041 mmol, 1.5 equiv) in portions at room temperature under nitrogen atmosphere. The resulting mixture was stirred for 3 h at room temperature. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The crude product was purified by Prep-HPLC to afford 3-(5-(6-fluoroindoline-1-carbonyl)-1-oxoisoindolin-2-yl)piperidine-2,6-dione (27 mg, 9%) as an off-white solid.

LC-MS (ES, m/z)=408 [M+1]⁺. ¹H NMR (400 MHz, DMSO-d₆) δ 11.03 (s, 1H), 7.84 (d, J=7.7 Hz, 2H), 7.73 (d, J=7.9 Hz, 1H), 7.34-7.26 (m, 1H), 6.90 (s, 1H), 5.16 (dd, J=13.3, 5.1 Hz, 1H), 4.54 (d, J=17.6 Hz, 1H), 4.41 (d, J=17.6 Hz, 1H), 4.05 (t, J=8.2 Hz, 2H), 3.07 (t, J=8.4 Hz, 2H), 2.98-2.86 (m, 1H), 2.66-2.57 (m, 1H), 2.47-2.36 (m, 1H), 2.08-2.00 (m, 1H).

### Example 33

### Synthesis of 3-(1-oxo-5-(4-(trifluoromethoxy)indoline-1-carbonyl)isoindolin-2-yl)piperidine-2,6-dione

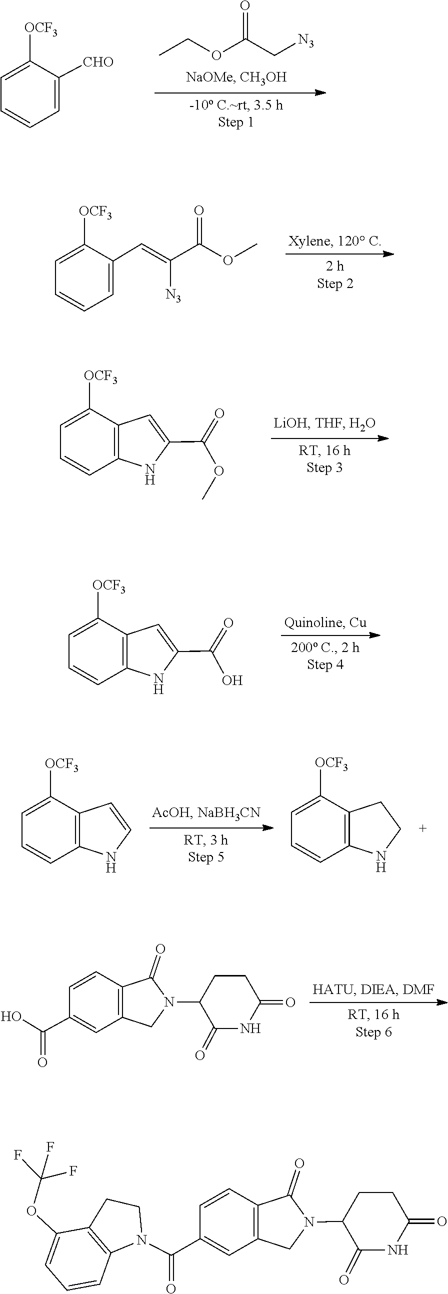

Step 1. Preparation of methyl (Z)-2-azido-3-(2-(trifluoromethoxy)phenyl)acrylate. To a stirred solution of NaOMe (1.14 g, 21.040 mmol, 4 equiv) in MeOH (10 ml) were added 2-(trifluoromethoxy)benzaldehyde (1 g, 5.260 mmol, 1 equiv) and ethyl 2-azidoacetate (2.72 g, 21.040 mmol, 4 equiv) in MeOH (10 ml) dropwise at −10° C. under nitrogen atmosphere. The resulting mixture was stirred for 1.5 h at −10° C. and additional 2 h at room temperature under nitrogen atmosphere. The reaction was quenched with Water/Ice at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (10:1) to afford methyl (Z)-2-azido-3-(2-(trifluoromethoxy)phenyl)acrylate (590 mg, 39%) as a yellow solid.

Step 2. Preparation of methyl 4-(trifluoromethoxy)-1H-indole-2-carboxylate. A solution of methyl (Z)-2-azido-3-(2-(trifluoromethoxy)phenyl)acrylate (590 mg, 2.054 mmol, 1 equiv) in Xylene (12 mL, 97.204 mmol, 47.32 equiv) was stirred for 2 h at 120° C. under nitrogen atmosphere. The mixture was allowed to cool down to room temperature. The resulting mixture was concentrated under vacuum. The residue was purified by silica gel column chromatography, eluted with PE/EA (10:1) to afford methyl 4-(trifluoromethoxy)-1H-indole-2-carboxylate (500 mg, 94%) as a yellow solid.

Step 3. Preparation of 4-(trifluoromethoxy)-1H-indole-2-carboxylic acid. To a stirred solution of methyl 4-(trifluoromethoxy)-1H-indole-2-carboxylate (500 mg, 1.929 mmol, 1 equiv) in THF (8 mL) was added LiOH (231.0 mg, 9.645 mmol, 5 equiv) in H₂O (2 mL, 111.019 mmol, 57.55 equiv) dropwise at room temperature under nitrogen atmosphere. The resulting mixture was stirred for overnight at room temperature under nitrogen atmosphere. The resulting mixture was diluted with water (10 mL) and THF was concentrated under vacuum. The mixture was acidified to pH 4 with 1N HCl (aq.). The precipitated solids were collected by filtration and washed with water (3×5 mL) to afford 4-(trifluoromethoxy)-1H-indole-2-carboxylic acid (400 mg, 85%) as a yellow solid.

Step 4. Preparation of 4-(trifluoromethoxy)-1H-indole. A solution of 4-(trifluoromethoxy)-1H-indole-2-carboxylic acid (300 mg, 1.224 mmol, 1 equiv) and Cu (54.4 mg, 0.857 mmol, 0.7 equiv) in Quinoline (6 mL) was stirred for 2 h at 200° C. under nitrogen atmosphere. The mixture was allowed to cool down to room temperature. The resulting mixture was diluted with water (30 mL) and extracted with CH₂Cl₂ (3×20 mL). The combined organic layers were washed with brine (2×20 mL), dried over anhydrous Na₂SO₄. After filtration, the filtrate was concentrated under reduced pressure. The residue was purified by reverse-phase flash chromatography to afford 4-(trifluoromethoxy)-1H-indole (164 mg, 67%) as a yellow oil.

Step 5. Preparation of 4-(trifluoromethoxy)indoline. To a stirred solution of 4-(trifluoromethoxy)-1H-indole (164 mg, 0.815 mmol, 1 equiv) in AcOH (2 mL) was added NaBH₃CN (256.17 mg, 4.075 mmol, 5 equiv) in portions at room temperature under nitrogen atmosphere. The resulting mixture was stirred for 3 h at room temperature under nitrogen atmosphere. The reaction was quenched with water (30 mL) at 0° C. and extracted with EtOAc (3×20 mL). The combined organic layers were washed with brine (2×20 mL), dried over anhydrous Na₂SO₄. After filtration, the filtrate was concentrated under reduced pressure to afford 4-(trifluoromethoxy)indoline (106 mg crude) as a yellow oil.

Step 6. Preparation of 3-(1-oxo-5-(4-(trifluoromethoxy)indoline-1-carbonyl)isoindolin-2-yl)piperidine-2,6-dione. To a stirred mixture of 2-(2,6-dioxopiperidin-3-yl)-1-oxoisoindole-5-carboxylic acid (100 mg, 0.347 mmol, 1.00 equiv), 4-(trifluoromethoxy)indoline (106 mg, 0.520 mmol, 1.5 equiv) and DIEA (179.4 mg, 1.388 mmol, 4 equiv) in DMF (2 mL) was added HATU (197.8 mg, 0.520 mmol, 1.5 equiv) in portions at room temperature under nitrogen atmosphere. The resulting mixture was stirred for overnight at room temperature under nitrogen atmosphere. The resulting mixture was diluted with water (10 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(1-oxo-5-(4-(trifluoromethoxy)indoline-1-carbonyl)isoindolin-2-yl)piperidine-2,6-dione (26 mg, 16%) as an off-white solid.

LC-MS (ES, m/z)=473.9 [M+1]⁺. ¹H NMR (400 MHz, DMSO-d₆) δ 11.02 (s, 1H), 8.10 (d, J 31.0 Hz, 1H), 7.87-7.82 (m, 2H), 7.73 (d, J=7.9 Hz, 1H), 7.36 (s, 1H), 7.08 (d, J=8.2 Hz, 1H), 5.16 (dd, J=13.3, 5.1 Hz, 1H), 4.59-4.37 (m, 2H), 4.08 (t, J=8.3 Hz, 2H), 3.14 (t, J=8.3 Hz, 2H), 2.93 (ddd, J=17.9, 13.6, 5.4 Hz, 1H), 2.61 (d, J=17.2 Hz, 1H), 2.48-2.32 (m, 1H), 2.09-1.99 (m, 1H).

### Example 34

### Synthesis of 3-(5-((S)-3-isopropyl-1,2,3,4-tetrahydroisoquinoline-2-carbonyl)-1-oxoisoindolin-2-yl)piperidine-2,6-dione

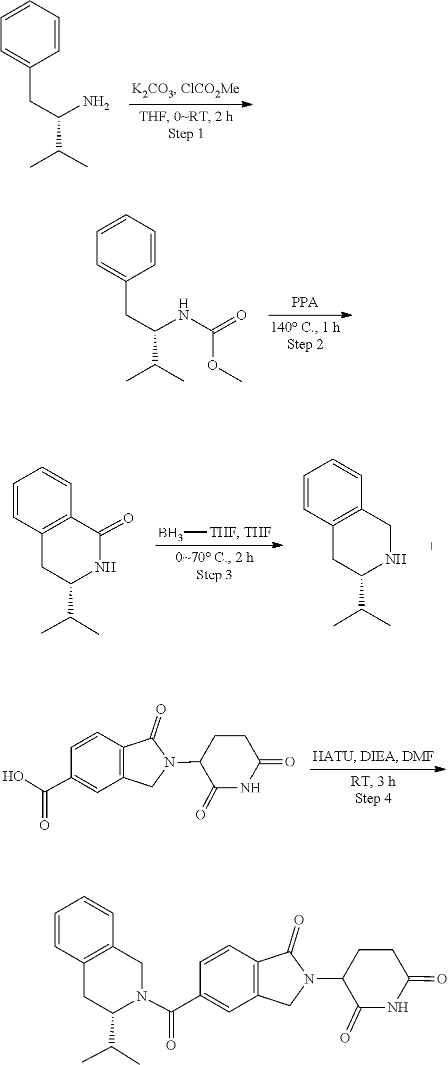

Step 1. Preparation of methyl (S)-(3-methyl-1-phenylbutan-2-yl)carbamate. To a stirred solution of (2S)-3-methyl-1-phenylbutan-2-amine (200 mg, 1.225 mmol, 1 equiv) and K₂CO₃ (254 mg, 1.838 mmol, 1.5 equiv) in THF (3 mL) was added methyl chloroformate (347 mg, 3.675 mmol, 3 equiv) dropwise at 0° C. under nitrogen atmosphere. The resulting mixture was stirred for 2 h at room temperature under nitrogen atmosphere. The resulting mixture was filtered, the filter cake was washed with THF (3×10 mL). The filtrate was concentrated under reduced pressure to afford methyl (S)-(3-methyl-1-phenylbutan-2-yl)carbamate (240 mg, 89%) as a brown oil.

Step 2. Preparation of (S)-3-isopropyl-3,4-dihydroisoquinolin-1(2H)-one. Into a 50 mL round-bottom flask were added methyl (S)-(3-methyl-1-phenylbutan-2-yl)carbamate (200 mg, 0.904 mmol, 1 equiv) and PPA (2 g, 17.388 mmol, 19.24 equiv) at room temperature. The resulting mixture was stirred for 1 h at 140° C. under nitrogen atmosphere. The mixture was allowed to cool down to room temperature. The reaction was quenched by the addition of Water/Ice (20 mL) at 0° C. The resulting mixture was diluted with water (10 mL). The resulting mixture was extracted with EtOAc (3×20 mL). The combined organic layers were washed with brine (2×10 mL), dried over anhydrous Na₂SO₄. After filtration, the filtrate was concentrated under reduced pressure to afford (S)-3-isopropyl-3,4-dihydroisoquinolin-1 (2H)-one (150 mg, 88%) as a brown oil.

Step 3. Preparation of (S)-3-isopropyl-1,2,3,4-tetrahydroisoquinoline. To a stirred solution of (S)-3-isopropyl-3,4-dihydroisoquinolin-1(2H)-one (200 mg, 1.057 mmol, 1 equiv) in THF (2 mL) was added BH₃-THF (1 mL, 0.95 equiv) dropwise at 0° C. under nitrogen atmosphere. The resulting mixture was stirred for overnight at 70° C. under nitrogen atmosphere. The reaction was quenched by the addition of MeOH (20 mL) at 0° C. The resulting mixture was concentrated under reduced pressure. The mixture was acidified to pH 3 with conc. HCl. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse-phase flash chromatography to afford (S)-3-isopropyl-1,2,3,4-tetrahydroisoquinoline (80 mg, 43%) as a yellow oil.

Step 4. Preparation of 3-(5-((S)-3-isopropyl-1,2,3,4-tetrahydroisoquinoline-2-carbonyl)-1-oxoisoindolin-2-yl)piperidine-2,6-dione. To a stirred solution of (S)-3-isopropyl-1,2,3,4-tetrahydroisoquinoline (30 mg, 0.171 mmol, 1 equiv), 2-(2,6-dioxopiperidin-3-yl)-1-oxoisoindole-5-carboxylic acid (49 mg, 0.171 mmol, 1 equiv) and DIEA (66 mg, 0.513 mmol, 3 equiv) in DMF (1 mL) was added HATU (97.6 mg, 0.257 mmol, 1.5 equiv) in portions at room temperature under nitrogen atmosphere. The resulting mixture was stirred for 3 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-((S)-3-isopropyl-1,2,3,4-tetrahydroisoquinoline-2-carbonyl)-1-oxoisoindolin-2-yl)piperidine-2,6-dione (10 mg, 13%) as an off-white solid.

LC-MS (ES, m/z)=446.05 [M+1]⁺. ¹H NMR (400 MHz, DMSO-d₆) δ 11.02 (s, 1H), 7.82 (dd, J=7.8, 3.1 Hz, 1H), 7.72-7.63 (m, 1H), 7.59-7.48 (m, 1H), 7.28-7.07 (m, 4H), 5.25 (d, J=18.1 Hz, 1H), 5.15 (dd, J=13.1, 4.7 Hz, 1H), 4.62-4.49 (m, 3H), 4.49-4.34 (m, 1H), 3.51 (s, 1H), 3.20 (d, J=16.3 Hz, 1H), 2.99-2.84 (m, 2H), 2.62 (d, J=17.2 Hz, 1H), 2.47-2.35 (m, 1H), 2.04 (s, 1H), 1.75 (s, 1H), 0.99 (ddd, J=19.0, 6.6, 3.2 Hz, 3H), 0.82-0.65 (m, 3H).

### Example 35

### Synthesis of 3-(5-((R)-1-methyl-1,2,3,4-tetrahydroisoquinoline-2-carbonyl)-1-oxoisoindolin-2-yl)piperidine-2,6-dione

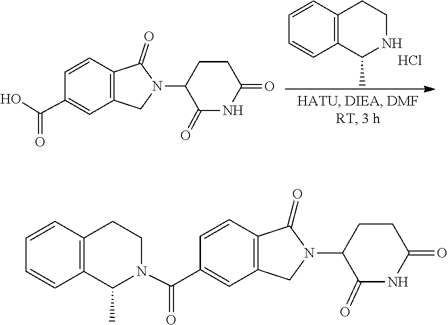 [SMILES: O=C1CCC(N2Cc3cc(C(=O)O)ccc3C2=O)C(=O)N1 ; C[C@H]1NCCc2ccccc21 ; Cl ; C[C@@H]1c2ccccc2CCN1C(=O)c1ccc2c(c1)CN(C1CCC(=O)NC1=O)C2=O]

Step 1. Preparation of 3-(5-((R)-1-methyl-1,2,3,4-tetrahydroisoquinoline-2-carbonyl)-1-oxoisoindolin-2-yl)piperidine-2,6-dione. A solution of (1R)-1-methyl-1,2,3,4-tetrahydroisoquinoline hydrochloride (95.6 mg, 0.520 mmol, 1.5 equiv), 2-(2,6-dioxopiperidin-3-yl)-1-oxoisoindole-5-carboxylic acid (100 mg, 0.347 mmol, 1.00 equiv) and DIEA (134.5 mg, 1.041 mmol, 3 equiv) in DMF (3 mL) was stirred for 5 min at room temperature under air atmosphere. To the above mixture was added HATU (197.9 mg, 0.520 mmol, 1.5 equiv) in portions over 5 min at room temperature. The resulting mixture was stirred for additional 3 h at room temperature. The reaction was quenched by the addition of Water (1 mL) at 0° C. The resulting mixture was concentrated under reduced pressure. The residue was purified by reverse-phase flash chromatography to afford 3-(5-((R)-1-methyl-1,2,3,4-tetrahydroisoquinoline-2-carbonyl)-1-oxoisoindolin-2-yl)piperidine-2,6-dione (62 mg, 43%) as an off-white solid.

LC-MS (ES, m/z)=418 [M+1]⁺. ¹H NMR (400 MHz, DMSO-d₆) δ 11.01 (s, 1H), 7.81 (d, J=7.7 Hz, 1H), 7.66 (d, J=20.0 Hz, 1H), 7.58-7.48 (m, 1H), 7.34-7.03 (m, 4H), 5.61 (d, J=6.9 Hz, 1H), 5.15 (dd, J=13.2, 5.1 Hz, 1H), 4.71-4.33 (m, 3H), 3.59 (d, J=12.8 Hz, 1H), 3.45 (t, J=12.5 Hz, 1H), 2.98-2.82 (m, 2H), 2.73-2.57 (m, 2H), 2.42 (d, J=13.3 Hz, 1H), 2.06-1.99 (m, 1H), 1.55-1.44 (m, 3H).

### Example 36

### Synthesis of 3-(5-((S)-1-methyl-1,2,3,4-tetrahydroisoquinoline-2-carbonyl)-1-oxoisoindolin-2-yl)piperidine-2,6-dione

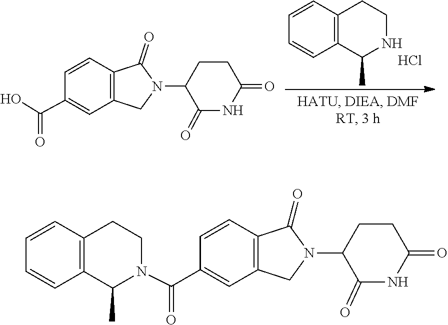 [SMILES: O=C1CCC(N2Cc3cc(C(=O)O)ccc3C2=O)C(=O)N1 ; C[C@@H]1NCCc2ccccc21 ; Cl ; C[C@H]1c2ccccc2CCN1C(=O)c1ccc2c(c1)CN(C1CCC(=O)NC1=O)C2=O]

Step 1. Preparation of 3-(5-((S)-1-methyl-1,2,3,4-tetrahydroisoquinoline-2-carbonyl)-1-oxoisoindolin-2-yl)piperidine-2,6-dione. To a stirred solution of (1S)-1-methyl-1,2,3,4-tetrahydroisoquinoline hydrochloride (50 mg, 0.272 mmol, 1 equiv), 2-(2,6-dioxopiperidin-3-yl)-1-oxoisoindole-5-carboxylic acid (79 mg, 0.272 mmol, 1 equiv) and DIEA (175.9 mg, 1.360 mmol, 5 equiv) in DMF (1 mL) was added HATU (155.3 mg, 0.408 mmol, 1.5 equiv) in portions at room temperature under nitrogen atmosphere. The resulting mixture was stirred for 3 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-((S)-1-methyl-1,2,3,4-tetrahydroisoquinoline-2-carbonyl)-1-oxoisoindolin-2-yl)piperidine-2,6-dione (78 mg, 68%) as an off-white solid.

LC-MS (ES, m/z)=418.05 [M+1]⁺. ¹H NMR (400 MHz, DMSO-d₆) δ 11.02 (s, 1H), 7.81 (d, J=7.8 Hz, 1H), 7.71-7.46 (m, 1H), 7.32 (d, J=7.5 Hz, 1H), 7.19 (d, J=27.4 Hz, 4H), 5.61 (d, J=7.2 Hz, 1H), 5.15 (dd, J=13.2, 5.1 Hz, 1H), 4.56-4.46 (m, 2H), 4.39 (dd, J=17.6, 9.2 Hz, 1H), 3.59 (d, J=13.0 Hz, 1H), 3.46 (d, J=12.1 Hz, 1H), 3.00-2.82 (m, 2H), 2.79-2.53 (m, 2H), 2.51-2.32 (m, 1H), 2.12-1.94 (m, 1H), 1.61-1.28 (m, 3H).

### Example 37

### Synthesis of 3-(5-(azepane-1-carbonyl)-1-oxoisoindolin-2-yl)piperidine-2,6-dione

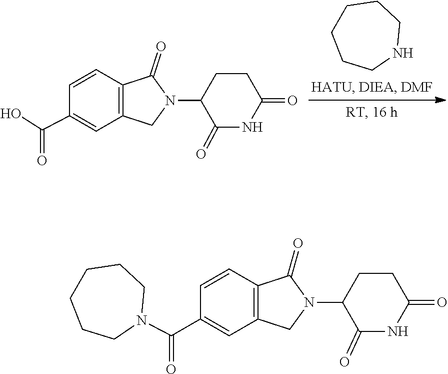 [SMILES: O=C1CCC(N2Cc3cc(C(=O)O)ccc3C2=O)C(=O)N1 ; C1CCCNCC1 ; O=C1CCC(N2Cc3cc(C(=O)N4CCCCCC4)ccc3C2=O)C(=O)N1]

Step 1. Preparation of 3-(5-(azepane-1-carbonyl)-1-oxoisoindolin-2-yl)piperidine-2,6-dione. To a stirred mixture of 2-(2,6-dioxopiperidin-3-yl)-1-oxoisoindole-5-carboxylic acid (60 mg, 0.208 mmol, 1.00 equiv), hexamethyleneimine (31.0 mg, 0.312 mmol, 1.5 equiv) and DIEA (80.7 mg, 0.624 mmol, 3 equiv) in DMF (1.2 mL) was added HATU (118.7 mg, 0.312 mmol, 1.5 equiv) in portions at room temperature under nitrogen atmosphere. The resulting mixture was stirred for overnight at room temperature under nitrogen atmosphere. The resulting mixture was diluted with water (10 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-(azepane-1-carbonyl)-1-oxoisoindolin-2-yl)piperidine-2,6-dione (37 mg, 49%) as an off-white solid.

LC-MS (ES, m/z)=370 [M+1]⁺. ¹H NMR (400 MHz, DMSO-d₆) δ 11.01 (s, 1H), 7.77 (d, J=7.8 Hz, 1H), 7.60 (s, 1H), 7.47 (dd, J=7.7, 1.5 Hz, 1H), 5.13 (dd, J=13.3, 5.0 Hz, 1H), 4.55-4.32 (m, 2H), 3.58 (td, J=5.6, 1.9 Hz, 2H), 3.29 (s, 2H), 2.99-2.84 (m, 1H), 2.61 (d, J=17.5 Hz, 1H), 2.41 (qd, J=13.1, 4.3 Hz, 1H), 2.02 (ddq, J=11.0, 5.9, 3.6, 3.0 Hz, 1H), 1.73 (q, J 6.0 Hz, 2H), 1.63-1.47 (m, 6H).

### Example 38

### Synthesis of 3-(5-((S)-3-methyl-1,2,3,4-tetrahydroisoquinoline-2-carbonyl)-1-oxoisoindolin-2-yl)piperidine-2,6-dione

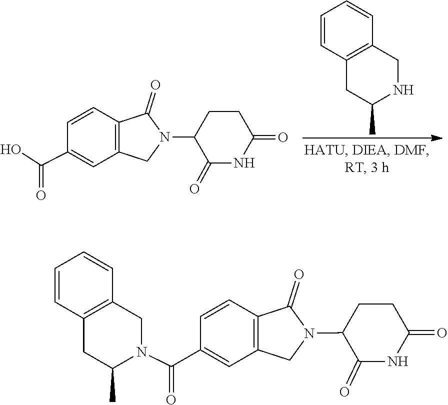 [SMILES: O=C1CCC(N2Cc3cc(C(=O)O)ccc3C2=O)C(=O)N1 ; C[C@H]1Cc2ccccc2CN1 ; C[C@H]1Cc2ccccc2CN1C(=O)c1ccc2c(c1)CN(C1CCC(=O)NC1=O)C2=O]

Step 1. Preparation of 3-(5-((S)-3-methyl-1,2,3,4-tetrahydroisoquinoline-2-carbonyl)-1-oxoisoindolin-2-yl)piperidine-2,6-dione. To a stirred solution of (3 S)-3-methyl-1,2,3,4-tetrahydroisoquinoline (50 mg, 0.340 mmol, 1 equiv), 2-(2,6-dioxopiperidin-3-yl)-1-oxoisoindole-5-carboxylic acid (98 mg, 0.340 mmol, 1 equiv) and DIEA (132 mg, 1.020 mmol, 3 equiv) in DMF (1 mL) was added HATU (193.7 mg, 0.510 mmol, 1.5 equiv) in portions at room temperature under nitrogen atmosphere. The resulting mixture was stirred for 3 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-((S)-3-methyl-1,2,3,4-tetrahydroisoquinoline-2-carbonyl)-1-oxoisoindolin-2-yl)piperidine-2,6-dione (44 mg, 31%) as an off-white solid.

LC-MS (ES, m/z)=418 [M+1]⁺. ¹H NMR (400 MHz, DMSO-d₆) δ 11.02 (s, 1H), 7.82 (d, J=7.7 Hz, 1H), 7.68 (s, 1H), 7.55 (d, J=7.8 Hz, 1H), 7.19 (s, 4H), 5.15 (dd, J=13.2, 5.1 Hz, 2H), 4.53 (dd, J=17.6, 4.3 Hz, 3H), 4.40 (dd, J=17.6, 4.9 Hz, 1H), 3.12 (dd, J=15.9, 5.7 Hz, 1H), 2.93 (ddd, J=18.1, 13.5, 5.4 Hz, 1H), 2.61 (d, J=16.9 Hz, 2H), 2.47-2.36 (m, 1H), 2.07-1.99 (m, 1H), 1.07 (s, 3H).

### Example 39

### Synthesis of 3-(5-((R)-3-isopropyl-1,2,3,4-tetrahydroisoquinoline-2-carbonyl)-1-oxoisoindolin-2-yl)piperidine-2,6-dione

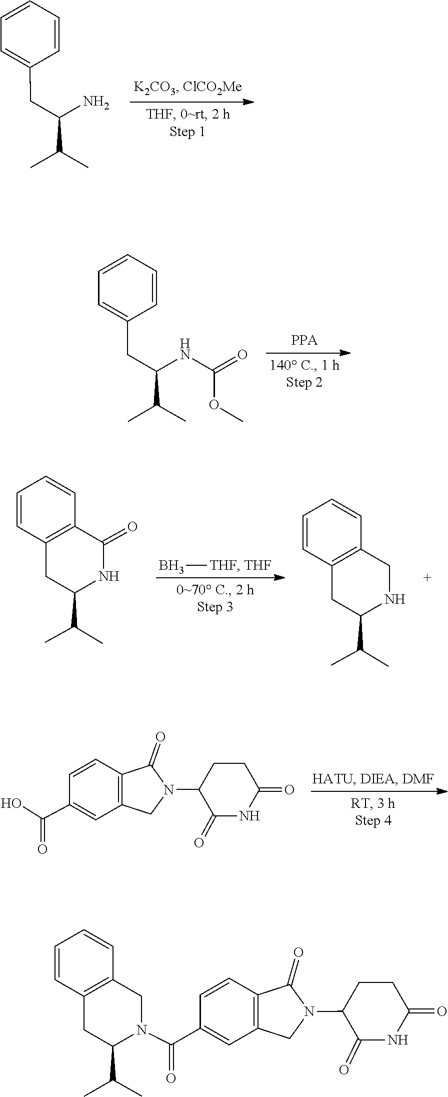

Step 1. Preparation of methyl (R)-(3-methyl-1-phenylbutan-2-yl)carbamate. To a stirred solution of (2R)-3-methyl-1-phenylbutan-2-amine (400 mg, 2.450 mmol, 1 equiv) and K₂CO₃ (1 g, 7.350 mmol, 3 equiv) in THF (4 mL) was added methyl carbonochloridate (463.0 mg, 4.900 mmol, 2 equiv) dropwise at 0° C. under nitrogen atmosphere. The resulting mixture was stirred for overnight at room temperature under nitrogen atmosphere. The resulting mixture was filtered, the filter cake was washed with THF (3×10 mL). The filtrate was concentrated under reduced pressure to afford methyl (R)-(3-methyl-1-phenylbutan-2-yl)carbamate (200 mg, 37%) as a yellow oil.

Step 2. Preparation of (R)-3-isopropyl-3,4-dihydroisoquinolin-1(2H)-one. Into a 50 mL round-bottom flask were added methyl (R)-(3-methyl-1-phenylbutan-2-yl)carbamate (200 mg, 0.904 mmol, 1 equiv) and PPA (1 g, 8.694 mmol, 9.62 equiv) at room temperature. The resulting mixture was stirred for 1 h at 140° C. under nitrogen atmosphere. The mixture was allowed to cool down to room temperature. The reaction was quenched with Water/Ice at 0° C. The resulting mixture was diluted with water (20 mL). The resulting mixture was extracted with EtOAc (3×15 mL). The combined organic layers were washed with brine (2×10 mL), dried over anhydrous Na₂SO₄. After filtration, the filtrate was concentrated under reduced pressure to afford (R)-3-isopropyl-3,4-dihydroisoquinolin-1(2H)-one (100 mg, 58%) as a brown oil.

Step 3. Preparation of (R)-3-isopropyl-1,2,3,4-tetrahydroisoquinoline. To a stirred solution of (R)-3-isopropyl-3,4-dihydroisoquinolin-1(2H)-one (200 mg, 1.057 mmol, 1 equiv) in THF (2 mL) was added BH₃-THF (5.28 mL, 5 equiv) dropwise at 0° C. under nitrogen atmosphere. The resulting mixture was stirred for overnight at 70° C. under nitrogen atmosphere. The reaction was quenched by the addition of MeOH (20 mL) at 0° C. The resulting mixture was concentrated under reduced pressure. The mixture was acidified to pH 3 with conc. HCl. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford (R)-3-isopropyl-1,2,3,4-tetrahydroisoquinoline (80 mg, 43%) as a yellow oil.

Step 4. Preparation of 3-(5-((R)-3-isopropyl-1,2,3,4-tetrahydroisoquinoline-2-carbonyl)-1-oxoisoindolin-2-yl)piperidine-2,6-dione. To a stirred solution of (R)-3-isopropyl-1,2,3,4-tetrahydroisoquinoline (40 mg, 0.228 mmol, 1 equiv), 2-(2,6-dioxopiperidin-3-yl)-1-oxoisoindole-5-carboxylic acid (66 mg, 0.228 mmol, 1 equiv) and DIEA (88.4 mg, 0.684 mmol, 3 equiv) in DMF (1 mL) was added HATU (130.1 mg, 0.342 mmol, 1.5 equiv) in portions at room temperature under nitrogen atmosphere. The resulting mixture was stirred for 3 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-((R)-3-isopropyl-1,2,3,4-tetrahydroisoquinoline-2-carbonyl)-1-oxoisoindolin-2-yl)piperidine-2,6-dione (3.4 mg, 3.3%) as a colorless oil.

LC-MS (ES, m/z)=446.10 [M+1]⁺. ¹H NMR (400 MHz, DMSO-d₆) δ 11.02 (s, 1H), 7.82 (d, J=7.7 Hz, 1H), 7.72-7.63 (m, 1H), 7.59-7.48 (m, 1H), 7.30-6.91 (m, 4H), 5.30-5.10 (m, 2H), 4.63-4.34 (m, 3H), 4.21 (d, J=18.2 Hz, 1H), 3.51 (dd, J=10.2, 4.9 Hz, 1H), 3.19 (dd, J=16.3, 5.3 Hz, 1H), 3.00-2.84 (m, 3H), 2.66-2.56 (m, 1H), 2.49-2.34 (m, 1H), 2.07-2.00 (m, 1H), 1.70 (ddt, J=30.3, 10.4, 7.0 Hz, 1H), 0.99 (dd, J=19.1, 6.6 Hz, 3H), 0.69 (d, J=6.5 Hz, 3H).

### Example 40

### Synthesis of 3-(5-((R)-4-methyl-1,2,3,4-tetrahydroisoquinoline-2-carbonyl)-1-oxoisoindolin-2-yl)piperidine-2,6-dione

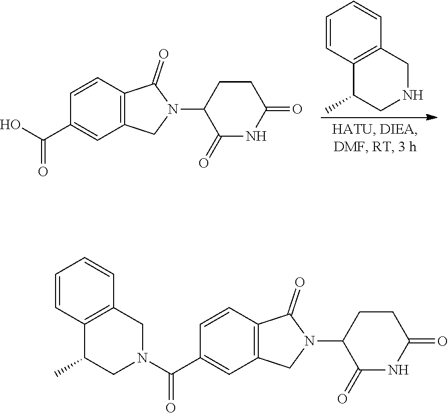

Step 1. Preparation of 3-(5-((R)-4-methyl-1,2,3,4-tetrahydroisoquinoline-2-carbonyl)-1-oxoisoindolin-2-yl)piperidine-2,6-dione. To a stirred solution of (4R)-4-methyl-1,2,3,4-tetrahydroisoquinoline (50 mg, 0.340 mmol, 1 equiv), 2-(2,6-dioxopiperidin-3-yl)-1-oxoisoindole-5-carboxylic acid (98 mg, 0.340 mmol, 1 equiv) and DIEA (131.7 mg, 1.020 mmol, 3 equiv) in DMF (1 mL) was added HATU (194 mg, 0.510 mmol, 1.5 equiv) in portions at room temperature under nitrogen atmosphere. The resulting mixture was stirred for 3 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-((R)-4-methyl-1,2,3,4-tetrahydroisoquinoline-2-carbonyl)-1-oxoisoindolin-2-yl)piperidine-2,6-dione (40 mg, 28%) as an off-white solid.

LC-MS (ES, m/z)=418 [M+1]⁺. ¹H NMR (400 MHz, DMSO-d₆) δ 11.02 (s, 1H), 7.82 (d, J=7.8 Hz, 1H), 7.68 (s, 1H), 7.55 (d, J=7.7 Hz, 1H), 7.24 (d, J=16.5 Hz, 3H), 7.07 (d, J=43.2 Hz, 1H), 5.15 (dd, J=13.3, 5.1 Hz, 1H), 5.00-4.66 (m, 1H), 4.52 (d, J=17.8 Hz, 2H), 4.39 (d, J=17.5 Hz, 1H), 3.81 (d, J=64.2 Hz, 1H), 3.60 (d, J=13.1 Hz, 1H), 2.93 (ddd, J=18.2, 14.0, 5.8 Hz, 2H), 2.66-2.57 (m, 1H), 2.46-2.35 (m, 1H), 2.06-1.99 (m, 1H), 1.29 (s, 1H), 1.11 (d, J=6.7 Hz, 2H).

### Example 41

### Synthesis of 3-(1-oxo-5-(2,3,4,5-tetrahydro-1H-benzo[b]azepine-1-carbonyl)isoindolin-2-yl)piperidine-2,6-dione

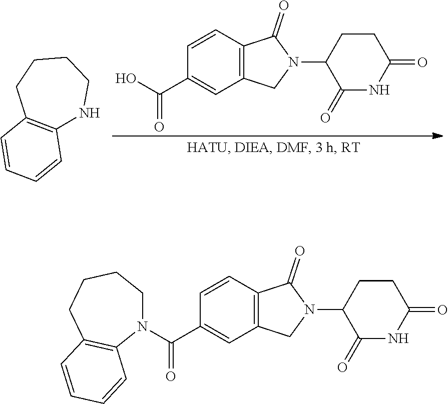

Step 1. Preparation of 3-(1-oxo-5-(2,3,4,5-tetrahydro-1H-benzo[b]azepine-1-carbonyl)isoindolin-2-yl)piperidine-2,6-dione. A solution of 2,3,4,5-tetrahydro-1H-1-benzazepine (23 mg, 0.156 mmol, 1.5 equiv), DIEA (40 mg, 0.312 mmol, 3 equiv) and 2-(2,6-dioxopiperidin-3-yl)-1-oxoisoindole-5-carboxylic acid (30 mg, 0.104 mmol, 1.00 equiv) in DMF (1 mL) was stirred for 5 min at room temperature under air atmosphere. To the above mixture was added HATU (59.4 mg, 0.156 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The residue was purified by reverse-phase flash chromatography to afford 3-(1-oxo-5-(2,3,4,5-tetrahydro-1H-benzo[b]azepine-1-carbonyl)isoindolin-2-yl)piperidine-2,6-dione (23 mg, 53%) as an off-white solid.

LC-MS (ES, m/z)=418 [M+1]⁺. ¹H NMR (400 MHz, DMSO-d₆) δ 10.97 (d, J=4.4 Hz, 1H), 7.53-7.44 (m, 2H), 7.29 (dd, J=7.5, 1.5 Hz, 1H), 7.16 (dd, J=15.6, 7.9 Hz, 1H), 7.08 (td, J=7.5, 1.3 Hz, 1H), 6.93 (q, J=6.8 Hz, 1H), 6.75 (t, J=6.6 Hz, 1H), 5.06 (dt, J=11.2, 5.2 Hz, 1H), 4.84 (d, J=13.5 Hz, 1H), 4.45-4.14 (m, 2H), 3.03 (t, J=13.0 Hz, 1H), 2.88 (d, J=7.4 Hz, 2H), 2.71 (d, J=9.9 Hz, 1H), 2.57 (d, J=16.8 Hz, 1H), 2.41-2.33 (m, 1H), 2.02 (s, 2H), 1.89 (t, J=4.6 Hz, 2H), 1.41 (d, J=12.6 Hz, 1H).

### Example 42

### Synthesis of 3-(1-oxo-5-(2,3,4,5-tetrahydro-1H-benzo[c]azepine-2-carbonyl)isoindolin-2-yl)piperidine-2,6-dione

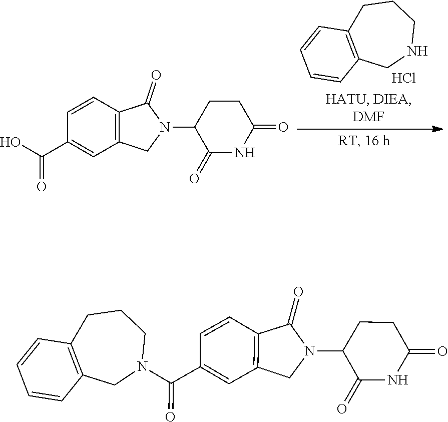 [SMILES: O=C1CCC(N2Cc3cc(C(=O)O)ccc3C2=O)C(=O)N1 ; c1ccc2c(c1)CCCNC2 ; Cl ; O=C1CCC(N2Cc3cc(C(=O)N4CCCc5ccccc5C4)ccc3C2=O)C(=O)N1]

Step 1. Preparation of 3-(1-oxo-5-(2,3,4,5-tetrahydro-1H-benzo[c]azepine-2-carbonyl)isoindolin-2-yl)piperidine-2,6-dione. To a stirred mixture of 2,3,4,5-tetrahydro-1H-2-benzazepine hydrochloride (50 mg, 0.272 mmol, 1.1 equiv), 2-(2,6-dioxopiperidin-3-yl)-1-oxoisoindole-5-carboxylic acid (71 mg, 0.247 mmol, 1 equiv) and DIEA (128 mg, 0.989 mmol, 4 equiv) in DMF (1 mL) was added HATU (141 mg, 0.371 mmol, 1.5 equiv) in portions at room temperature under nitrogen atmosphere. The resulting mixture was stirred for overnight at room temperature under nitrogen atmosphere. The resulting mixture was diluted with water (10 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(1-oxo-5-(2,3,4,5-tetrahydro-1H-benzo[c]azepine-2-carbonyl)isoindolin-2-yl)piperidine-2,6-dione (19 mg, 18%) as an off-white solid.

LC-MS (ES, m/z)=418.05 [M+1]⁺. ¹H NMR (400 MHz, DMSO-d₆) δ 11.01 (d, J=8.6 Hz, 1H), 7.74 (d, J=7.5 Hz, 1H), 7.52 (s, 1H), 7.42-7.31 (m, 2H), 7.28-7.10 (m, 3H), 5.14 (td, J=14.8, 13.4, 5.0 Hz, 1H), 4.71 (s, 1H), 4.46 (d, J=17.7 Hz, 2H), 4.35 (dd, J=17.6, 9.7 Hz, 1H), 3.94 (s, 1H), 3.59 (t, J=5.4 Hz, 1H), 3.05-2.83 (m, 3H), 2.60 (d, J=17.4 Hz, 1H), 2.46-2.32 (m, 1H), 2.10-1.94 (m, 1H), 1.82 (s, 1H), 1.66 (s, 1H).

### Example 43

### Synthesis of 3-(1-oxo-5-(2,3,4,5-tetrahydro-1H-benzo[d]azepine-3-carbonyl)isoindolin-2-yl)piperidine-2,6-dione

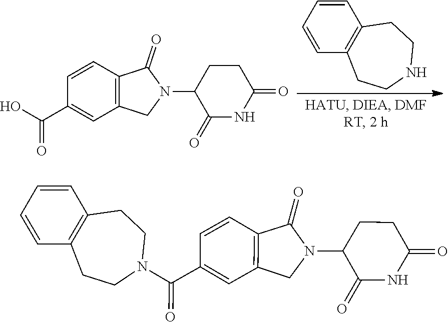

Step 1. Preparation of 3-(1-oxo-5-(2,3,4,5-tetrahydro-1H-benzo[d]azepine-3-carbonyl)isoindolin-2-yl)piperidine-2,6-dione. To a stirred mixture of 2,3,4,5-tetrahydro-1H-3-benzazepine (25.5 mg, 0.173 mmol, 1 equiv) and 2-(2,6-dioxopiperidin-3-yl)-1-oxoisoindole-5-carboxylic acid (49.9 mg, 0.173 mmol, 1 equiv) in DMF (1 mL) were added DIEA (67.2 mg, 0.519 mmol, 3 equiv) and HATU (98.8 mg, 0.259 mmol, 1.5 equiv) at room temperature. The resulting mixture was stirred for 2 h at room temperature. The resulting mixture was extracted with EtOAc (3×10 mL). The combined organic layers were washed with brine (2×5 mL), dried over anhydrous Na₂SO₄. After filtration, the filtrate was concentrated under reduced pressure. The residue was purified by reverse-phase flash chromatography to afford 3-(1-oxo-5-(2,3,4,5-tetrahydro-1H-benzo[d]azepine-3-carbonyl)isoindolin-2-yl)piperidine-2,6-dione (38 mg, 51%) as a white solid.

LC-MS (ES, m/z)=418 [M+1]⁺. ¹H NMR (400 MHz, DMSO-d₆) δ 11.01 (s, 1H), 7.80 (dd, J=7.8, 0.8 Hz, 1H), 7.61 (s, 1H), 7.49 (dd, J=7.7, 1.4 Hz, 1H), 7.26-7.09 (m, 4H), 5.15 (dd, J=13.3, 5.1 Hz, 1H), 4.52 (d, J=17.5 Hz, 1H), 4.38 (d, J=17.5 Hz, 1H), 3.76 (s, 2H), 3.01 (s, 2H), 2.92 (ddd, J=18.0, 13.5, 5.4 Hz, 3H), 2.61 (d, J=17.2 Hz, 1H), 2.40 (td, J=13.2, 4.5 Hz, 1H), 2.02 (d, J=5.7 Hz, 1H), 1.24 (s, 1H).

### Example 44

### Synthesis of 3-(5-((R)-1-isopropyl-1,2,3,4-tetrahydroisoquinoline-2-carbonyl)-1-oxoisoindolin-2-yl)piperidine-2,6-dione

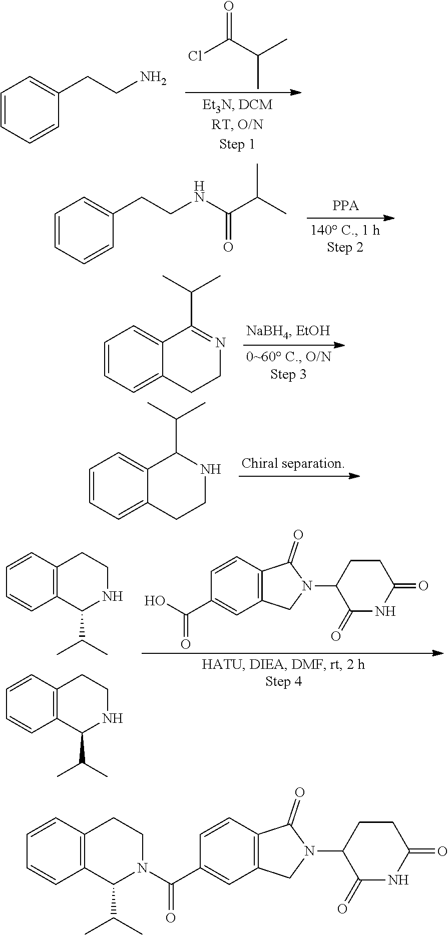

Step 1. Preparation of N-phenethylisobutyramide. To a stirred mixture of 2-phenylethylamine (1 g, 8.252 mmol, 1 equiv) and triethylamine (2.51 g, 24.756 mmol, 3 equiv) in DCM (15 mL) were added propanoyl chloride, 2-methyl-(1.14 g, 10.728 mmol, 1.3 equiv) at 0° C. The resulting mixture was stirred for overnight at room temperature. The resulting mixture was concentrated under reduced pressure. The residue was purified by silica gel column chromatography, eluted with PE/EA (5:1) to afford N-phenethylisobutyramide (1.5 g, 95%) as a yellow oil.

Step 2. Preparation of 1-isopropyl-3,4-dihydroisoquinoline. To a stirred mixture of N-phenethylisobutyramide (1.5 g, 7.842 mmol, 1 equiv) and PPA (15 mL) at room temperature. The resulting mixture was stirred for 1 h at 140° C. The mixture was neutralized to pH 7 with saturated NaHCO₃ (aq.). The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by reverse-phase flash chromatography to afford 1-isopropyl-3,4-dihydroisoquinoline (1.1 g, 81%) as a yellow oil.

Step 3. Preparation of 1-isopropyl-1,2,3,4-tetrahydroisoquinoline. To a stirred mixture of 1-isopropyl-3,4-dihydroisoquinoline (900 mg, 5.195 mmol, 1 equiv) in EtOH (9.47 mL) were added NaBH₄ (393 mg, 10.390 mmol, 2 equiv) in portions at 0° C. The resulting mixture was stirred for overnight at 60° C. The reaction was quenched by the addition of Water (10 mL) at 0° C. The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by reverse-phase flash chromatography to afford 1-isopropyl-1,2,3,4-tetrahydroisoquinoline (280 mg, 31%) as a white solid.

### Step 4. Chiral Separation

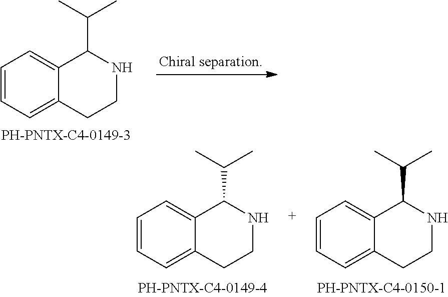

Then the isomers were purified by prep-CHIRIL-HPLC with the following conditions: column: Lux 3u Cellulose-4 4.6*100 mm, 3 μm; Mobile Phase B: IPA (10 mMNH3); Flow rate: 2 mL/min; Gradient: isocratic % B. This resulted in (1R)—1-isopropyl-1,2,3,4-tetrahydroisoquinoline (40 mg, 14.28%) as a white solid. This resulted in (1S)-1-isopropyl-1,2,3,4-tetrahydroisoquinoline (40 mg, 14.28%) as a white solid.

Step 5. Preparation of 3-(5-((R)-1-isopropyl-1,2,3,4-tetrahydroisoquinoline-2-carbonyl)-1-oxoisoindolin-2-yl)piperidine-2,6-dione. To a stirred mixture of (R)-1-isopropyl-1,2,3,4-tetrahydroisoquinoline (40 mg, 0.228 mmol, 1 equiv) and 2-(2,6-dioxopiperidin-3-yl)-1-oxoisoindole-5-carboxylic acid (66 mg, 0.228 mmol, 1 equiv) in DMF (1 mL) were added DIEA (89 mg, 0.684 mmol, 3 equiv) and HATU (130 mg, 0.342 mmol, 1.5 equiv) at room temperature. The resulting mixture was stirred for 2 h at room temperature. The resulting mixture was extracted with EtOAc (3×10 mL). The combined organic layers were washed with brine (2×5 mL), dried over anhydrous Na₂SO₄. After filtration, the filtrate was concentrated under reduced pressure. The residue was purified by reverse-phase flash chromatography to afford 3-(5-((R)-1-isopropyl-1,2,3,4-tetrahydroisoquinoline-2-carbonyl)-1-oxoisoindolin-2-yl)piperidine-2,6-dione (53 mg, 51%) as an off-white solid. LC-MS (ES, m/z)=446.15 [M+1]⁺. ¹H NMR (400 MHz, DMSO-d₆) δ 11.01 (s, 1H), 7.80 (t, J=7.3 Hz, 1H), 7.65 (s, 1H), 7.51 (d, J=7.8 Hz, 1H), 7.31-7.26 (m, 1H), 7.26-7.12 (m, 3H), 5.35 (d, J=8.7 Hz, 1H), 5.14 (dd, J=13.3, 5.0 Hz, 1H), 4.51 (d, J=17.6 Hz, 1H), 4.38 (d, J=17.5 Hz, 1H), 3.66-3.47 (m, 2H), 3.05 (t, J=7.1 Hz, 1H), 2.99-2.75 (m, 3H), 2.61 (d, J=16.6 Hz, 1H), 2.40 (dt, J=13.6, 6.6 Hz, 1H), 2.16 (dq, J=14.3, 7.0 Hz, 1H), 2.06-1.99 (m, 1H), 1.03 (dd, J=9.4, 6.7 Hz, 5H), 0.86 (dd, J=6.5, 2.7 Hz, 1H), 0.73-0.67 (m, 1H).

### Example 45

### Synthesis of 3-(5-((S)-1-isopropyl-1,2,3,4-tetrahydroisoquinoline-2-carbonyl)-1-oxoisoindolin-2-yl)piperidine-2,6-dione

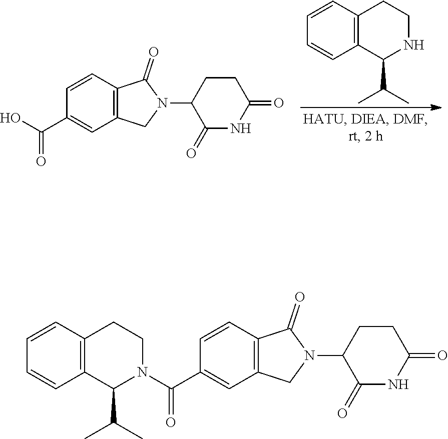 [SMILES: O=C1CCC(N2Cc3cc(C(=O)O)ccc3C2=O)C(=O)N1 ; CC(C)[C@@H]1NCCc2ccccc21 ; CC(C)[C@H]1c2ccccc2CCN1C(=O)c1ccc2c(c1)CN(C1CCC(=O)NC1=O)C2=O]

Step 1. Preparation of 3-(5-((S)-1-isopropyl-1,2,3,4-tetrahydroisoquinoline-2-carbonyl)-1-oxoisoindolin-2-yl)piperidine-2,6-dione. To a stirred mixture of (S)-1-isopropyl-1,2,3,4-tetrahydroisoquinoline (Step 4, Example 44, 40 mg, 0.228 mmol, 1 equiv) and 2-(2,6-dioxopiperidin-3-yl)-1-oxo-3H-isoindole-5-carboxylic acid (66 mg, 0.228 mmol, 1 equiv) in DMF (1 mL) were added DIEA (89 mg, 0.684 mmol, 3 equiv) and HATU (130 mg, 0.342 mmol, 1.5 equiv) at room temperature. The resulting mixture was stirred for 2 h at room temperature. The resulting mixture was extracted with EtOAc (3×10 mL). The combined organic layers were washed with brine (2×5 mL), dried over anhydrous Na₂SO₄. After filtration, the filtrate was concentrated under reduced pressure. The residue was purified by reverse-phase flash chromatography to afford 3-(5-((S)-1-isopropyl-1,2,3,4-tetrahydroisoquinoline-2-carbonyl)-1-oxoisoindolin-2-yl)piperidine-2,6-dione (59 mg, 57%) as an off-white solid.

LC-MS (ES, m/z)=446.20 [M+1]⁺. ¹H NMR (400 MHz, DMSO-d₆) δ 11.02 (s, 1H), 7.80 (t, J=7.3 Hz, 1H), 7.65 (s, 1H), 7.51 (dd, J=7.7, 1.9 Hz, 1H), 7.30-7.17 (m, 4H), 5.35 (d, J=8.7 Hz, 1H), 5.15 (dd, J=13.4, 5.1 Hz, 1H), 4.52-4.35 (m, 2H), 3.66-3.51 (m, 2H), 3.05 (t, J=7.1 Hz, 1H), 2.92 (ddd, J=18.4, 13.6, 5.5 Hz, 1H), 2.87-2.75 (m, 2H), 2.65-2.57 (m, 1H), 2.42 (tt, J=14.4, 7.0 Hz, 1H), 2.23-2.08 (m, 1H), 2.03 (d, J=6.1 Hz, 1H), 1.03 (dd, J=9.3, 6.7 Hz, 5H), 0.86 (dd, J=6.7, 2.8 Hz, 1H), 0.70 (dd, J=6.6, 2.3 Hz, 1H).

### Example 46

### Synthesis of N-(((3R)-2-(2-(2,6-dioxopiperidin-3-yl)-1-oxoisoindoline-5-carbonyl)-1,2,3,4-tetrahydroisoquinolin-3-yl)methyl)acetamide

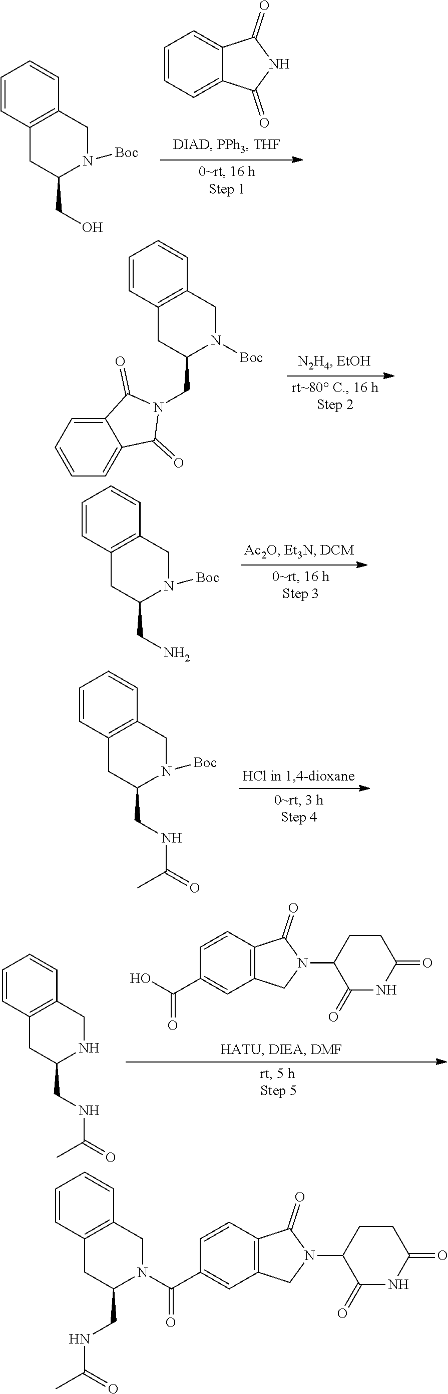

Step 1. Preparation of tert-butyl (R)-3-((1,3-dioxoisoindolin-2-yl)methyl)-3,4-dihydroisoquinoline-2(1H)-carboxylate. To a stirred solution of tert-butyl (R)-3-(hydroxymethyl)-3,4-dihydro-1H-isoquinoline-2-carboxylate (1 g, 3.797 mmol, 1 equiv), phthalimide (0.67 g, 4.556 mmol, 1.2 equiv) and PPh₃ (1.49 g, 5.696 mmol, 1.5 equiv) in THF (10 mL) was added DIAD (1.5 mL) dropwise at 0° C. under nitrogen atmosphere. The resulting mixture was stirred for overnight at room temperature under nitrogen atmosphere. The resulting mixture was concentrated under reduced pressure. The reaction was quenched by the addition of Water/Ice (10 mL) at 0° C. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford tert-butyl (R)-3-((1,3-dioxoisoindolin-2-yl)methyl)-3,4-dihydroisoquinoline-2(1H)-carboxylate (809 mg, 54%) as an off-white solid.

Step 2. Preparation of tert-butyl (R)-3-(aminomethyl)-3,4-dihydroisoquinoline-2(1H)-carboxylate. To a stirred solution of tert-butyl (R)-3-((1,3-dioxoisoindolin-2-yl)methyl)-3,4-dihydroisoquinoline-2(1H)-carboxylate (750 mg, 1.911 mmol, 1 equiv) in ethyl alcohol (7 mL) was added Hydrazine hydrate (937.43 mg, 19.110 mmol, 10 equiv) at room temperature under nitrogen atmosphere. The resulting mixture was stirred for overnight at 80° C. under nitrogen atmosphere. The mixture was allowed to cool down to room temperature. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford tert-butyl (R)-3-(aminomethyl)-3,4-dihydroisoquinoline-2(1H)-carboxylate (200 mg, 40%) as a colorless solid.

Step 3. Preparation of tert-butyl (R)-3-(acetamidomethyl)-3,4-dihydroisoquinoline-2(1H)-carboxylate. To a stirred solution of tert-butyl (R)-3-(aminomethyl)-3,4-dihydroisoquinoline-2(1H)-carboxylate (250 mg, 0.953 mmol, 1 equiv) and Et₃N (289 mg, 2.859 mmol, 3 equiv) in DCM (3 mL) was added Ac₂O (0.2 mL) dropwise at 0° C. under nitrogen atmosphere. The resulting mixture was stirred for 3 h at room temperature under nitrogen atmosphere. The resulting mixture was concentrated under reduced pressure. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford tert-butyl (R)-3-(acetamidomethyl)-3,4-dihydroisoquinoline-2(1H)-carboxylate (250 mg, 86%) as an off-white solid.

Step 4. Preparation of (R)—N-((1,2,3,4-tetrahydroisoquinolin-3-yl)methyl)acetamide. To a stirred solution of tert-butyl (R)-3-(acetamidomethyl)-3,4-dihydroisoquinoline-2(1H)-carboxylate (300 mg, 0.986 mmol, 1 equiv) in MeOH (3 mL) was added HCl in 1,4-dioxane (1 mL) dropwise at 0° C. under nitrogen atmosphere. The resulting mixture was stirred for 3 h at room temperature under nitrogen atmosphere. The resulting mixture was concentrated under reduced pressure. The crude product was used in the next step directly without further purification.

Step 5. Preparation of N-(((3R)-2-(2-(2,6-dioxopiperidin-3-yl)-1-oxoisoindoline-5-carbonyl)-1,2,3,4-tetrahydroisoquinolin-3-yl)methyl)acetamide. To a stirred solution of (R)—N-((1,2,3,4-tetrahydroisoquinolin-3-yl)methyl)acetamide (75 mg, 0.367 mmol, 1 equiv), 2-(2,6-dioxopiperidin-3-yl)-1-oxoisoindole-5-carboxylic acid (70 mg, 0.242 mmol, 0.66 equiv) and DIEA (190 mg, 1.468 mmol, 4 equiv) in DMF (1 mL) was added HATU (209.4 mg, 0.550 mmol, 1.5 equiv) in portions at room temperature under nitrogen atmosphere. The resulting mixture was stirred for 5 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford N-(((3R)-2-(2-(2,6-dioxopiperidin-3-yl)-1-oxoisoindoline-5-carbonyl)-1,2,3,4-tetrahydroisoquinolin-3-yl)methyl)acetamide (33 mg, 19%) as an off-white solid.

LC-MS (ES, m/z)=475.15 [M+1]⁺. ¹H NMR (400 MHz, DMSO-d₆) δ 11.02 (s, 1H), 8.19-7.93 (m, 1H), 7.81 (dd, J=15.3, 7.6 Hz, 1H), 7.63 (d, J=7.3 Hz, 1H), 7.50 (d, J=8.1 Hz, 1H), 7.31-7.14 (m, 4H), 5.24 (d, J=18.1 Hz, 1H), 5.18-5.11 (m, 1H), 4.52 (s, 1H), 4.46-4.33 (m, 2H), 4.07 (s, 1H), 3.19 (d, J=16.7 Hz, 1H), 3.04-2.87 (m, 3H), 2.64 (t, J=18.5 Hz, 2H), 2.42 (dt, J=14.8, 7.4 Hz, 1H), 2.03 (d, J=12.2 Hz, 1H), 1.81 (d, J=26.8 Hz, 3H).

### Example 47

### Synthesis of N-(((2R)—1-(2-(2,6-dioxopiperidin-3-yl)-1-oxoisoindoline-5-carbonyl)indolin-2-yl methyl)acetamide

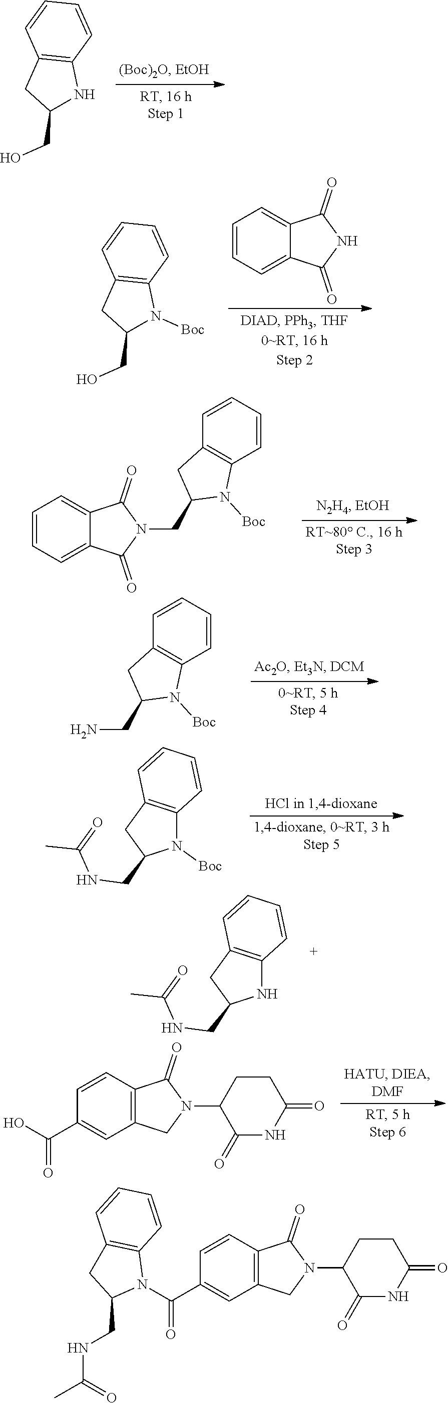

Step 1. Preparation of tert-butyl (R)-2-(hydroxymethyl)indoline-1-carboxylate. To a stirred solution of (R)-indolin-2-ylmethanol (950 mg, 6.368 mmol, 1 equiv) in EtOH (10 mL) was added di-tert-butyl dicarbonate (2.78 g, 12.736 mmol, 2 equiv) at room temperature under nitrogen atmosphere. The resulting mixture was stirred for overnight at room temperature under nitrogen atmosphere. The resulting mixture was concentrated under reduced pressure. The residue was purified by silica gel column chromatography, eluted with PE/EA (10:1) to afford tert-butyl (R)-2-(hydroxymethyl)indoline-1-carboxylate (1.5 g, 94%) as a brown oil.

Step 2. Preparation of tert-butyl (R)-2-((1,3-dioxoisoindolin-2-yl)methyl)indoline-1-carboxylate. To a stirred solution of tert-butyl (R)-2-(hydroxymethyl)indoline-1-carboxylate (1.5 g, 6.017 mmol, 1 equiv), phthalimide (1.33 g, 9.026 mmol, 1.5 equiv) and PPh₃ (2.37 g, 9.026 mmol, 1.5 equiv) in THF (10 mL) was added DIAD (1.82 g, 9.026 mmol, 1.5 equiv) dropwise at 0° C. under nitrogen atmosphere. The resulting mixture was stirred for overnight at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (5:1) to afford tert-butyl (R)-2-((1,3-dioxoisoindolin-2-yl)methyl)indoline-1-carboxylate (2 g, 88%) as an off-white solid.

Step 3. Preparation of tert-butyl (R)-2-(aminomethyl)indoline-1-carboxylate. To a stirred solution of tert-butyl (R)-2-((1,3-dioxoisoindolin-2-yl)methyl)indoline-1-carboxylate (1 g, 2.643 mmol, 1 equiv) in EtOH (10 mL) was added Hydrazine hydrate (0.82 g, 26.430 mmol, 10 equiv) at room temperature under nitrogen atmosphere. The resulting mixture was stirred for overnight at 80° C. under nitrogen atmosphere. The mixture was allowed to cool down to room temperature. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The crude product was used in the next step directly without further purification.

Step 4. Preparation of tert-butyl (R)-2-(acetamidomethyl)indoline-1-carboxylate. To a stirred solution of tert-butyl (R)-2-(aminomethyl)indoline-1-carboxylate (300 mg, 1.208 mmol, 1 equiv) and triethylamine (367 mg, 3.624 mmol, 3 equiv) in DCM (4 mL) was added Ac₂O (0.3 mL) dropwise at 0° C. under nitrogen atmosphere. The resulting mixture was stirred for 5 h at room temperature under nitrogen atmosphere. The resulting mixture was concentrated under reduced pressure. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford tert-butyl (R)-2-(acetamidomethyl)indoline-1-carboxylate (200 mg, 57%) as an off-white solid.

Step 5. Preparation of (R)—N-(indolin-2-ylmethyl)acetamide. To a stirred solution of tert-butyl (R)-2-(acetamidomethyl)indoline-1-carboxylate (200 mg, 0.689 mmol, 1 equiv) in 1,4-dioxane (2 mL) was added HCl (gas) in 1,4-dioxane (1 mL) dropwise at 0° C. under nitrogen atmosphere. The resulting mixture was stirred for 3 h at room temperature under nitrogen atmosphere. The resulting mixture was concentrated under reduced pressure to afford (R)—N-(indolin-2-ylmethyl)acetamide (100 mg, crude) as a brown oil. The resulting mixture was used in the next step directly without further purification.

Step 6. Preparation of N-(((2R)—1-(2-(2,6-dioxopiperidin-3-yl)-1-oxoisoindoline-5-carbonyl)indolin-2-yl)methyl)acetamide. To a stirred solution of (R)—N-(indolin-2-ylmethyl)acetamide (50 mg, 0.263 mmol, 1 equiv), 2-(2,6-dioxopiperidin-3-yl)-1-oxoisoindole-5-carboxylic acid (76 mg, 0.263 mmol, 1 equiv) and DIEA (102 mg, 0.789 mmol, 3 equiv) in DMF (1 mL) was added HATU (150 mg, 0.395 mmol, 1.5 equiv) in portions at room temperature under nitrogen atmosphere. The resulting mixture was stirred for 5 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The crude product was purified by Prep-HPLC to afford N-(((2R)—1-(2-(2,6-dioxopiperidin-3-yl)-1-oxoisoindoline-5-carbonyl)indolin-2-yl)methyl)acetamide (10 mg, 8.4%) as an off-white solid.

LC-MS (ES, m/z)=461.15 [M+1]⁺. ¹H NMR (400 MHz, DMSO-d₆) δ 11.01 (s, 1H), 8.97 (s, 1H), 7.97 (d, J=8.1 Hz, 1H), 7.92-7.75 (m, 3H), 7.26 (d, J=7.3 Hz, 1H), 7.16 (t, J=7.9 Hz, 1H), 7.01 (t, J=7.4 Hz, 1H), 5.14 (dd, J=13.3, 5.1 Hz, 1H), 4.68 (s, 1H), 4.54-4.34 (m, 2H), 3.29 (dd, J=16.0, 8.7 Hz, 2H), 3.06-2.85 (m, 2H), 2.61 (d, J=17.7 Hz, 1H), 2.43 (dd, J=13.2, 4.5 Hz, 1H), 2.34 (s, 3H), 2.08-1.97 (m, 1H).

### Example 48

### Synthesis of 3-(1-oxo-5-((S)-3-phenyllpyrrolidine-1-carbonyl)isoindolin-2-yl)piperidine-2,6-dione

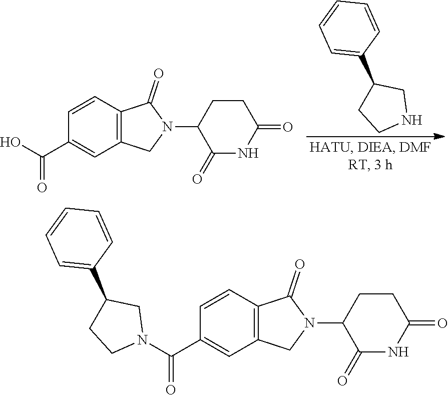

Step 1. Preparation of 3-(1-oxo-5-((S)-3-phenylpyrrolidine-1-carbonyl)isoindolin-2-yl)piperidine-2,6-dione. To a stirred mixture of (S)-3-phenylpyrrolidine (26 mg, 0.173 mmol, 1 equiv) and 2-(2,6-dioxopiperidin-3-yl)-1-oxoisoindole-5-carboxylic acid (50 mg, 0.173 mmol, 1.00 equiv) in DMF (1 mL) were added DIEA (67.3 mg, 0.519 mmol, 3 equiv) and HATU (98.9 mg, 0.259 mmol, 1.5 equiv) at room temperature. The resulting mixture was stirred for 3 h at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(1-oxo-5-((S)-3-phenylpyrrolidine-1-carbonyl)isoindolin-2-yl)piperidine-2,6-dione (45 mg, 61%) as a white solid.

LC-MS (ES, m/z)=418.15 [M+1]⁺. ¹H NMR (400 MHz, DMSO-d₆) δ 11.01 (d, J=6.1 Hz, 1H), 7.83-7.74 (m, 2H), 7.67 (d, J=7.9 Hz, 1H), 7.36 (d, J=4.3 Hz, 2H), 7.32-7.27 (m, 2H), 7.27-7.18 (m, 1H), 5.19-5.09 (m, 1H), 4.50 (t, J=17.0 Hz, 1H), 4.44-4.30 (m, 1H), 3.74 (dt, J=14.3, 8.0 Hz, 1H), 3.66-3.39 (m, 4H), 2.91 (ddd, J=18.8, 13.1, 6.1 Hz, 1H), 2.60 (d, J=17.5 Hz, 1H), 2.46-2.35 (m, 1H), 2.34-2.28 (m, 1H), 2.06 (dd, J=25.8, 12.4 Hz, 2H).

### Example 49

### Synthesis of 3-(1-oxo-5-((R)-3-phenylpyrrolidine-1-carbonyl)isoindolin-2-yl)piperidine-2,6-dione

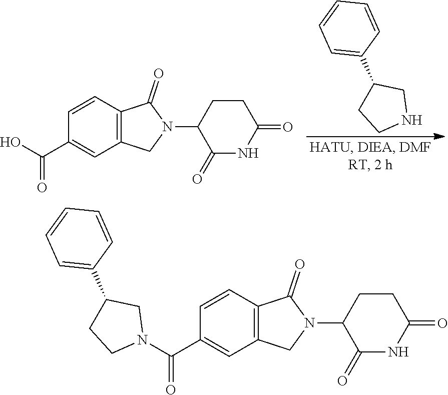

Step 1. Preparation of 3-(1-oxo-5-((R)-3-phenylpyrrolidine-1-carbonyl)isoindolin-2-yl)piperidine-2,6-dione. To a stirred mixture of (R)-3-phenylpyrrolidine (25.5 mg, 0.173 mmol, 1 equiv) and 2-(2,6-dioxopiperidin-3-yl)-1-oxoisoindole-5-carboxylic acid (50 mg, 0.173 mmol, 1.00 equiv) in DMF (1 mL) were added HATU (98.9 mg, 0.259 mmol, 1.5 equiv) and DIEA (67.3 mg, 0.519 mmol, 3 equiv) at room temperature. The resulting mixture was stirred for 2 h at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(1-oxo-5-((R)-3-phenylpyrrolidine-1-carbonyl)isoindolin-2-yl)piperidine-2,6-dione (22 mg, 29%) as a white solid.

LC-MS (ES, m/z)=418.15 [M+1]⁺. ¹H NMR (400 MHz, DMSO-d₆) δ 11.00 (d, J=5.9 Hz, 1H), 7.83-7.73 (m, 2H), 7.70-7.63 (m, 1H), 7.46-7.09 (m, 5H), 5.19-5.12 (m, 1H), 4.50 (t, J=16.8 Hz, 1H), 4.44-4.30 (m, 1H), 3.80-3.36 (m, 5H), 2.93-2.85 (m, 1H), 2.60 (d, J=17.1 Hz, 1H), 2.39-2.27 (m, 1H), 2.25-1.99 (m, 2H), 1.24 (s, 1H).

### Example 50

### Synthesis of 3-(5-((R)-3-(4-fluorophenyl)pyrrolidine-1-carbonyl)-1-oxoisoindolin-2-yl)piperidine-2,6-dione

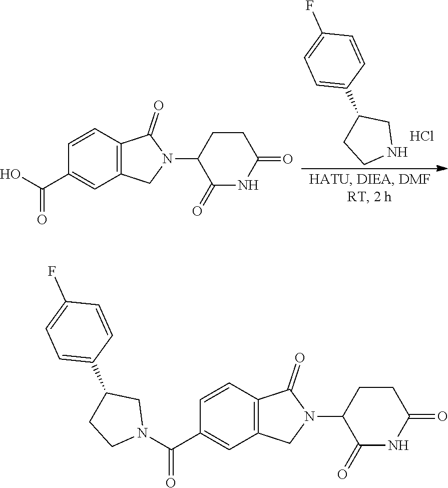 [SMILES: O=C1CCC(N2Cc3cc(C(=O)O)ccc3C2=O)C(=O)N1 ; Fc1ccc([C@H]2CCNC2)cc1 ; Cl ; O=C1CCC(N2Cc3cc(C(=O)N4CC[C@H](c5ccc(F)cc5)C4)ccc3C2=O)C(=O)N1]

Step 1. Preparation of 3-(5-((R)-3-(4-fluorophenyl)pyrrolidine-1-carbonyl)-1-oxoisoindolin-2-yl)piperidine-2,6-dione. To a stirred solution of (R)-3-(4-fluorophenyl)pyrrolidine hydrochloride (40 mg, 0.198 mmol, 1 equiv), 2-(2,6-dioxopiperidin-3-yl)-1-oxoisoindole-5-carboxylic acid (57 mg, 0.198 mmol, 1 equiv) and DIEA (77 mg, 0.594 mmol, 3 equiv) in DMF (1 mL) was added HATU (113.1 mg, 0.297 mmol, 1.5 equiv) in portions at room temperature under nitrogen atmosphere. The resulting mixture was stirred for 5 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-((R)-3-(4-fluorophenyl)pyrrolidine-1-carbonyl)-1-oxoisoindolin-2-yl)piperidine-2,6-dione (44 mg, 51%) as an off-white solid.

LC-MS (ES, m/z)=436 [M+1]⁺. ¹H NMR (400 MHz, DMSO-d₆) δ 11.01 (d, J=5.3 Hz, 1H), 7.83-7.74 (m, 2H), 7.66 (d, J=7.8 Hz, 1H), 7.44-7.36 (m, 1H), 7.36-7.28 (m, 1H), 7.23-7.08 (m, 2H), 5.15 (q, J=6.6, 6.2 Hz, 1H), 4.56-4.31 (m, 2H), 4.00 (dd, J=10.6, 6.6 Hz, 1H), 3.80-3.67 (m, 1H), 3.56 (ddd, J=24.3, 18.3, 8.5 Hz, 1H), 3.48-3.38 (m, 2H), 2.91 (ddd, J=18.4, 12.7, 5.4 Hz, 1H), 2.61 (d, J=17.2 Hz, 1H), 2.41 (t, J=13.0 Hz, 1H), 2.33-2.20 (m, 1H), 2.03 (tt, J=20.8, 11.2 Hz, 2H).

### Example 51

### Synthesis of 3-(5-((S)-3-(4-fluorophenyl)pyrrolidine-1-carbonyl)-1-oxoisoindolin-2-yl)piperidine-2,6-dione

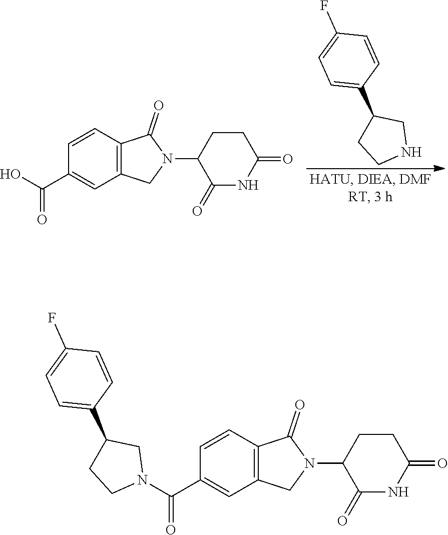 [SMILES: O=C1CCC(N2Cc3cc(C(=O)O)ccc3C2=O)C(=O)N1 ; Fc1ccc([C@@H]2CCNC2)cc1 ; O=C1CCC(N2Cc3cc(C(=O)N4CC[C@@H](c5ccc(F)cc5)C4)ccc3C2=O)C(=O)N1]

Step 1. Preparation of 3-(5-((S)-3-(4-fluorophenyl)pyrrolidine-1-carbonyl)-1-oxoisoindolin-2-yl)piperidine-2,6-dione. A solution of (S)-3-(4-fluorophenyl)pyrrolidine (29 mg, 0.173 mmol, 1 equiv), DIEA (67 mg, 0.519 mmol, 3 equiv) and 2-(2,6-dioxopiperidin-3-yl)-1-oxoisoindole-5-carboxylic acid (50 mg, 0.173 mmol, 1.00 equiv) in DMF (1 mL) was stirred for 3 min at room temperature. To the above mixture was added HATU (98.9 mg, 0.259 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The residue was purified by reverse-phase flash chromatography to afford 3-(5-((S)-3-(4-fluorophenyl)pyrrolidine-1-carbonyl)-1-oxoisoindolin-2-yl)piperidine-2,6-dione (37 mg, 49%) as an off-white solid.

LC-MS (ES, m/z)=436 [M+1]⁺. ¹H NMR (400 MHz, DMSO-d₆) δ 11.01 (d, J=5.5 Hz, 1H), 7.83-7.74 (m, 2H), 7.66 (d, J=7.9 Hz, 1H), 7.40 (dd, J=8.5, 5.6 Hz, 1H), 7.32 (dd, J=8.6, 5.5 Hz, 1H), 7.15 (dt, J=21.1, 8.8 Hz, 2H), 5.19-5.10 (m, 1H), 4.54-4.32 (m, 2H), 3.73 (d, J=9.7 Hz, 1H), 3.50 (ddt, J=46.2, 19.8, 9.7 Hz, 4H), 2.98-2.84 (m, 1H), 2.60 (d, J=17.6 Hz, 1H), 2.41 (t, J=13.1 Hz, 1H), 2.23 (s, 1H), 2.01 (d, J=10.9 Hz, 2H).

### Example 52

### Synthesis of 3-(1-oxo-5-((R)-3-(3-(trifluoromethyl)phenyl)pyrrolidine-1-carbonyl)isoindolin-2-yl)piperidine-2,6-dione

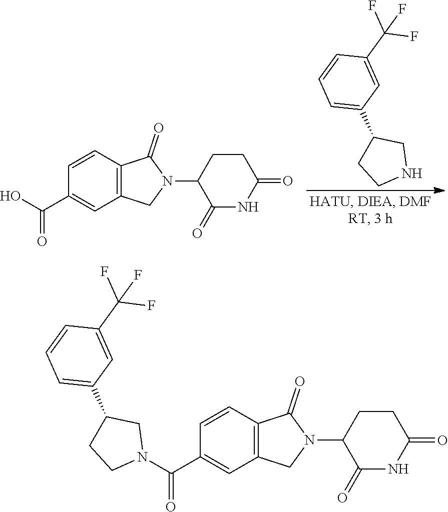 [SMILES: O=C1CCC(N2Cc3cc(C(=O)O)ccc3C2=O)C(=O)N1 ; FC(F)(F)c1cccc([C@H]2CCNC2)c1 ; O=C1CCC(N2Cc3cc(C(=O)N4CC[C@H](c5cccc(C(F)(F)F)c5)C4)ccc3C2=O)C(=O)N1]

Step 1. Preparation of 3-(1-oxo-5-((R)-3-(3-(trifluoromethyl)phenyl)pyrrolidine-1-carbonyl)isoindolin-2-yl)piperidine-2,6-dione. A solution of (R)-3-[3-(trifluoromethyl)phenyl]pyrrolidine (37.3 mg, 0.173 mmol, 1 equiv), DIEA (67.3 mg, 0.519 mmol, 3 equiv) and 2-(2,6-dioxopiperidin-3-yl)-1-oxoisoindole-5-carboxylic acid (50 mg, 0.173 mmol, 1.00 equiv) in DMF (1 mL) was stirred for 3 min at room temperature. To the above mixture was added HATU (98.9 mg, 0.259 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The residue was purified by reverse-phase flash chromatography to afford 3-(1-oxo-5-((R)-3-(3-(trifluoromethyl)phenyl)pyrrolidine-1-carbonyl)isoindolin-2-yl)piperidine-2,6-dione (36 mg, 43%) as an off-white solid.

LC-MS (ES, m/z)=486 [M+1]⁺. ¹H NMR (400 MHz, DMSO-d₆) δ 11.01 (d, J=6.7 Hz, 1H), 7.84-7.74 (m, 2H), 7.62 (dtd, J=35.6, 15.1, 8.1 Hz, 5H), 5.19-5.08 (m, 1H), 4.57-4.31 (m, 2H), 3.77 (t, J=5.8 Hz, 1H), 3.67-3.45 (m, 4H), 2.92 (ddt, J=18.0, 12.9, 5.7 Hz, 1H), 2.60 (d, J=17.4 Hz, 1H), 2.46-2.24 (m, 2H), 2.08 (d, J=45.6 Hz, 2H).

### Example 53

### Synthesis of 3-(1-oxo-5-((S)-3-(3-(trifluoromethyl)phenyl)pyrrolidine-1-carbonyl)isoindolin-2-yl)piperidine-2,6-dione

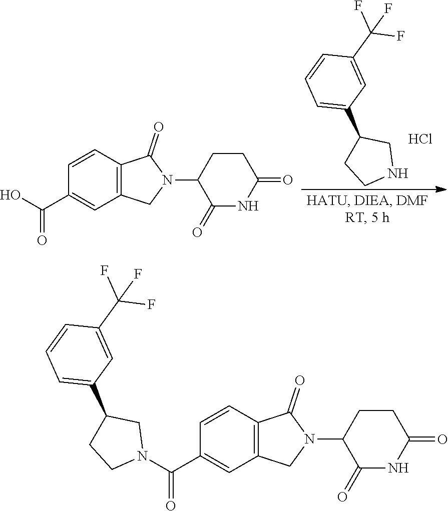 [SMILES: O=C1CCC(N2Cc3cc(C(=O)O)ccc3C2=O)C(=O)N1 ; FC(F)(F)c1cccc([C@@H]2CCNC2)c1 ; Cl ; O=C1CCC(N2Cc3cc(C(=O)N4CC[C@@H](c5cccc(C(F)(F)F)c5)C4)ccc3C2=O)C(=O)N1]

Step 1. Preparation of 3-(1-oxo-5-((S)-3-(3-(trifluoromethyl)phenyl)pyrrolidine-1-carbonyl)isoindolin-2-yl)piperidine-2,6-dione. To a stirred solution of (S)-3-[3-(trifluoromethyl)phenyl]pyrrolidine hydrochloride (40 mg, 0.159 mmol, 1 equiv), 2-(2,6-dioxopiperidin-3-yl)-1-oxoisoindole-5-carboxylic acid (45.8 mg, 0.159 mmol, 1 equiv) and DIEA (61.6 mg, 0.477 mmol, 3 equiv) in DMF (1 mL) was added HATU (90.7 mg, 0.238 mmol, 1.5 equiv) in portions at room temperature under nitrogen atmosphere. The resulting mixture was stirred for 5 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(1-oxo-5-((S)-3-(3-(trifluoromethyl)phenyl)pyrrolidine-1-carbonyl)isoindolin-2-yl)piperidine-2,6-dione (65 mg, 85%) as an off-white solid.

LC-MS (ES, m/z)=484.10 [M−1]⁻. ¹H NMR (400 MHz, DMSO-d₆) δ 11.01 (d, J=7.0 Hz, 1H), 7.84-7.74 (m, 2H), 7.74-7.50 (m, 5H), 5.14 (td, J=10.5, 8.0, 5.0 Hz, 1H), 4.57-4.31 (m, 2H), 3.82-3.72 (m, 1H), 3.67-3.45 (m, 4H), 2.92 (ddt, J=19.3, 13.4, 5.7 Hz, 1H), 2.60 (d, J 18.0 Hz, 1H), 2.47-2.26 (m, 2H), 2.19-1.96 (m, 2H).

### Example 54

### Synthesis of 3-(5-((R)-3-(3-fluorophenyl)pyrrolidine-1-carbonyl)-1-oxoisoindolin-2-yl)piperidine-2,6-dione

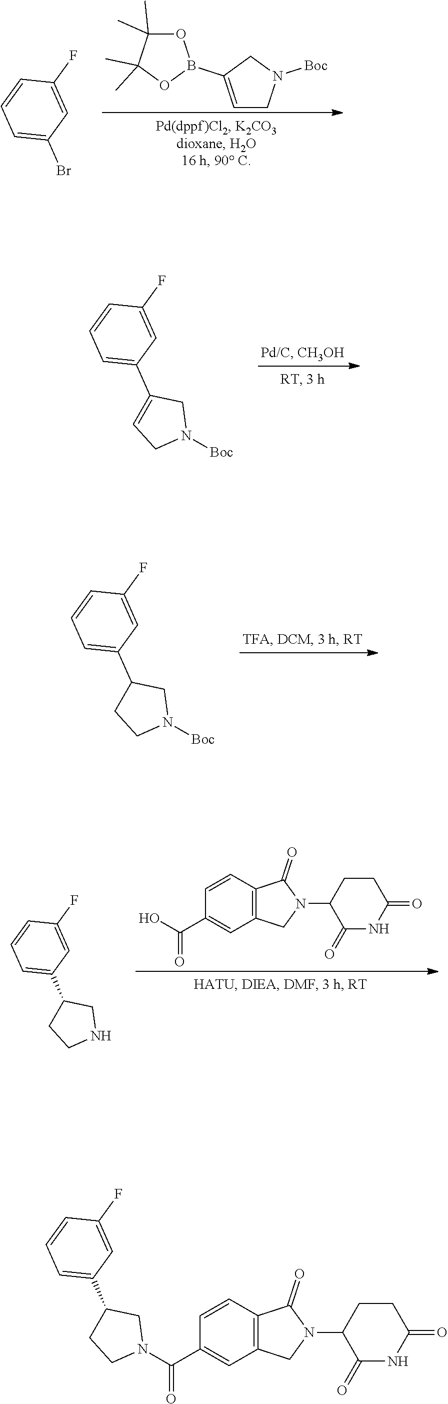

Step 1. Preparation of tert-butyl 3-(3-fluorophenyl)-2,5-dihydro-1H-pyrrole-1-carboxylate. A solution of 1-bromo-3-fluorobenzene (1 g, 5.714 mmol, 1 equiv), tert-butyl 3-(4,4,5,5-tetramethyl-1,3,2-dioxaborolan-2-yl)-2,5-dihydropyrrole-1-carboxylate (2.02 g, 6.857 mmol, 1.2 equiv) and K₂CO₃ (2.37 g, 17.142 mmol, 3 equiv) in dioxane (10 mL) and H₂O (0.1 mL) was stirred for 5 min at room temperature under nitrogen atmosphere. To the above mixture was added Pd(dppf)Cl₂ (0.42 g, 0.571 mmol, 0.1 equiv) in portions over 5 min at room temperature. The resulting mixture was stirred for additional 16 h at 90° C. under nitrogen atmosphere. The resulting mixture was filtered, the filter cake was washed with THF (3×30 mL). The filtrate was concentrated under reduced pressure. The residue was purified by reverse-phase flash chromatography to afford tert-butyl 3-(3-fluorophenyl)-2,5-dihydro-1H-pyrrole-1-carboxylate (1.1 g, 73%) as a brown solid.

Step 2. Preparation of tert-butyl 3-(3-fluorophenyl)pyrrolidine-1-carboxylate. A solution of tert-butyl 3-(3-fluorophenyl)-2,5-dihydro-1H-pyrrole-1-carboxylate (1 g, 3.798 mmol, 1 equiv) and Pd/C (0.40 g, 0.380 mmol, 0.1 equiv) 10% on Carbon (wetted with ca. 55% Water) in CH₃OH (8 mL) was stirred for 3 h at room temperature under hydrogen atmosphere. The resulting mixture was filtered, the filter cake was washed with THF (3×30 mL). The filtrate was concentrated under reduced pressure. The residue was purified by reverse-phase flash chromatography to afford tert-butyl 3-(3-fluorophenyl)pyrrolidine-1-carboxylate (1 g, 99%) as a light yellow oil.

Step 3. Preparation of (R)-3-(3-fluorophenyl)pyrrolidine. A solution of tert-butyl (R)-3-(3-fluorophenyl)pyrrolidine-1-carboxylate (100 mg, 0.377 mmol, 1 equiv) and TFA (0.3 mL) in DCM (1.5 mL) was stirred for 3 h at room temperature under nitrogen atmosphere. The reaction was quenched with Water at 0° C. The resulting mixture was concentrated under reduced pressure. The residue was purified by reverse-phase flash chromatography to afford (R)-3-(3-fluorophenyl)pyrrolidine (60 mg, 96%) as an off-white solid.

Step 4. Preparation of 3-(5-((R)-3-(3-fluorophenyl)pyrrolidine-1-carbonyl)-1-oxoisoindolin-2-yl)piperidine-2,6-dione. A solution of (R)-3-(3-fluorophenyl)pyrrolidine (28.7 mg, 0.173 mmol, 1.00 equiv), DIEA (67.3 mg, 0.519 mmol, 3 equiv) and 2-(2,6-dioxopiperidin-3-yl)-1-oxoisoindole-5-carboxylic acid (50 mg, 0.173 mmol, 1.00 equiv) in DMF (1.5 mL) was stirred for 3 min at room temperature. To the above mixture was added HATU (98.9 mg, 0.259 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The residue was purified by reverse-phase flash chromatography to afford 3-(5-((R)-3-(3-fluorophenyl)pyrrolidine-1-carbonyl)-1-oxoisoindolin-2-yl)piperidine-2,6-dione (18 mg, 24%) as an off-white solid.

LC-MS (ES, m/z)=436 [M+1]⁺. ¹H NMR (400 MHz, DMSO-d₆) δ 11.01 (d, J=5.6 Hz, 1H), 7.83-7.74 (m, 2H), 7.67 (d, J=7.8 Hz, 1H), 7.45-7.30 (m, 1H), 7.13 (ddt, J=42.0, 24.8, 8.4 Hz, 3H), 5.19-5.10 (m, 1H), 4.57-4.30 (m, 2H), 3.75 (dd, J=10.5, 7.3 Hz, 1H), 3.62 (d, J=9.0 Hz, 1H), 3.59-3.46 (m, 1H), 3.44 (d, J=9.4 Hz, 1H), 2.98-2.85 (m, 1H), 2.60 (d, J=17.3 Hz, 1H), 2.41 (t, J=12.2 Hz, 1H), 2.28 (d, J=23.5 Hz, 1H), 2.06 (dd, J=29.4, 9.6 Hz, 2H).

### Example 55

### Synthesis of 3-(5-((S)-3-(3-fluorophenyl)pyrrolidine-1-carbonyl)-1-oxoisoindolin-2-yl)piperidine-2,6-dione

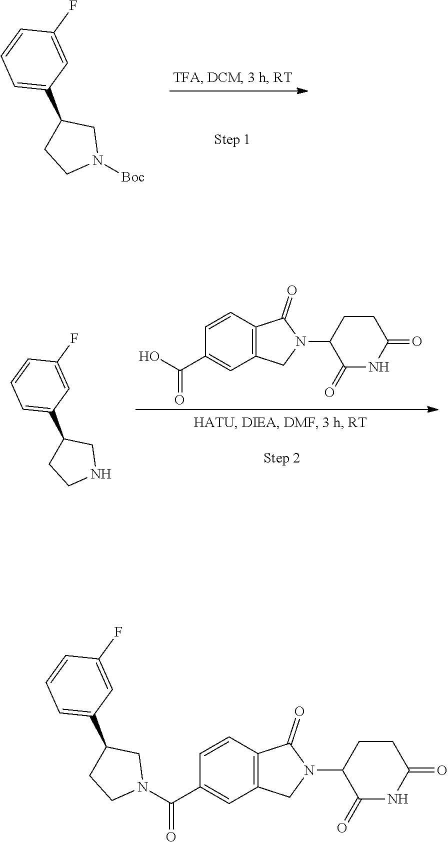 [SMILES: CC(C)(C)OC(=O)N1CC[C@@H](c2cccc(F)c2)C1 ; Fc1cccc([C@@H]2CCNC2)c1 ; O=C1CCC(N2Cc3cc(C(=O)O)ccc3C2=O)C(=O)N1 ; O=C1CCC(N2Cc3cc(C(=O)N4CC[C@@H](c5cccc(F)c5)C4)ccc3C2=O)C(=O)N1]

Step 1. Preparation of (S)-3-(3-fluorophenyl)pyrrolidine. A solution of tert-butyl (S)-3-(3-fluorophenyl)pyrrolidine-1-carboxylate (Example 54 step 2, 100 mg, 0.377 mmol, 1 equiv) and TFA (0.3 mL) in DCM (1.5 mL) was stirred for 3 h at room temperature under nitrogen atmosphere. The reaction was quenched with Water at 0° C. The resulting mixture was concentrated under reduced pressure. The residue was purified by reverse-phase flash chromatography to afford (S)-3-(3-fluorophenyl)pyrrolidine (60 mg, 96%) as an off-white solid.

Step 2. Preparation of 3-(5-((S)-3-(3-fluorophenyl)pyrrolidine-1-carbonyl)-1-oxoisoindolin-2-yl)piperidine-2,6-dione. A solution of (S)-3-(3-fluorophenyl)pyrrolidine (28.7 mg, 0.173 mmol, 1 equiv), DIEA (67.3 mg, 0.519 mmol, 3 equiv) and 2-(2,6-dioxopiperidin-3-yl)-1-oxoisoindole-5-carboxylic acid (50 mg, 0.173 mmol, 1.00 equiv) in DMF (1.5 mL) was stirred for 3 min at room temperature. To the above mixture was added HATU (98.9 mg, 0.259 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The residue was purified by reverse-phase flash chromatography to afford 3-(5-((S)-3-(3-fluorophenyl)pyrrolidine-1-carbonyl)-1-oxoisoindolin-2-yl)piperidine-2,6-dione (22 mg, 29%) as an off-white solid.

LC-MS (ES, m/z)=436 [M+1]⁺. ¹H NMR (400 MHz, DMSO-d₆) δ 11.01 (d, J=5.6 Hz, 1H), 7.83-7.74 (m, 2H), 7.66 (d, J=7.6 Hz, 1H), 7.45-7.26 (m, 1H), 7.24-7.00 (m, 3H), 5.19-5.09 (m, 1H), 4.57-4.31 (m, 2H), 3.75 (dd, J=10.2, 7.1 Hz, 1H), 3.64-3.57 (m, 1H), 3.52 (d, J=10.1 Hz, 2H), 3.46 (dd, J=17.1, 7.0 Hz, 1H), 2.91 (ddd, J=18.1, 11.8, 5.2 Hz, 1H), 2.60 (d, J=16.9 Hz, 1H), 2.41 (dd, J=17.3, 9.0 Hz, 1H), 2.24 (d, J=7.1 Hz, 1H), 2.04-1.99 (m, 2H).

### Example 56

### Synthesis of 3-(5-(5-chloro-7-fluoroindoline-1-carbonyl)-1-oxoisoindolin-2-yl)piperidine-2,6-dione

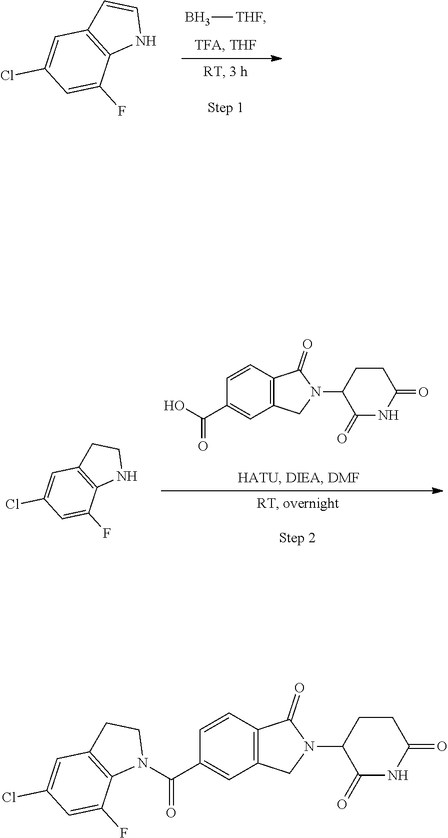

Step 1. Preparation of 5-chloro-7-fluoroindoline. A solution of 5-chloro-7-fluoro-1H-indole (400 mg, 2.359 mmol, 1 equiv) in THF (8.00 mL, 98.748 mmol, 41.86 equiv) was treated with PUP-39203 BH₃-THF (7.08 mL, 7.077 mmol, 3 equiv) for 30 min at 0° C. under nitrogen atmosphere followed by the addition of TFA (7.04 mL, 94.785 mmol, 40.18 equiv) dropwise at 0° C. The resulting mixture was stirred for overnight at room temperature under nitrogen atmosphere. The reaction was quenched by the addition of sat. NH₄Cl (aq.) (20 mL) at 0° C. The resulting mixture was extracted with EtOAc (3×20 mL). The combined organic layers were washed with brine (2×40 mL), dried over anhydrous Na₂SO₄. After filtration, the filtrate was concentrated under reduced pressure. The residue was purified by reverse-phase flash chromatography to afford 5-chloro-7-fluoroindoline (100 mg, 25%) as a yellow oil.

Step 2. Preparation of 3-(5-(5-chloro-7-fluoroindoline-1-carbonyl)-1-oxoisoindolin-2-yl)piperidine-2,6-dione. To a stirred mixture of 5-chloro-7-fluoroindoline (50 mg, 0.291 mmol, 1 equiv) and 2-(2,6-dioxopiperidin-3-yl)-1-oxoisoindole-5-carboxylic acid (83.9 mg, 0.291 mmol, 1 equiv) in DMF (1 mL, 12.922 mmol, 44.35 equiv) were added DIEA (112.9 mg, 0.873 mmol, 3 equiv) and HATU (166.1 mg, 0.436 mmol, 1.5 equiv) at room temperature. The resulting mixture was stirred for 3 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure.

The residue was purified by Prep-HPLC to afford in 3-(5-(5-chloro-7-fluoroindoline-1-carbonyl)-1-oxoisoindolin-2-yl)piperidine-2,6-dione (29 mg, 22%) as a light yellow solid. LC-MS (ES, m/z)=442.05 [M+1]⁺. ¹H NMR (400 MHz, DMSO-d₆) δ 11.02 (s, 1H), 7.94 (s, 1H), 7.87-7.77 (m, 2H), 7.31-7.24 (m, 2H), 5.15 (dd, J=13.3, 5.1 Hz, 1H), 4.54 (d, J=17.7 Hz, 1H), 4.41 (d, J=17.7 Hz, 1H), 4.14 (t, J=7.8 Hz, 2H), 3.13 (t, J=7.8 Hz, 2H), 2.92 (ddd, J=17.2, 13.6, 5.4 Hz, 1H), 2.66-2.57 (m, 1H), 2.49-2.34 (m, 2H), 2.10-1.99 (m, 1H).

### Example 57

### Synthesis of 3-(5-(5,7-difluoroindoline-1-carbonyl)-1-oxoisoindolin-2-yl)piperidine-2,6-dione

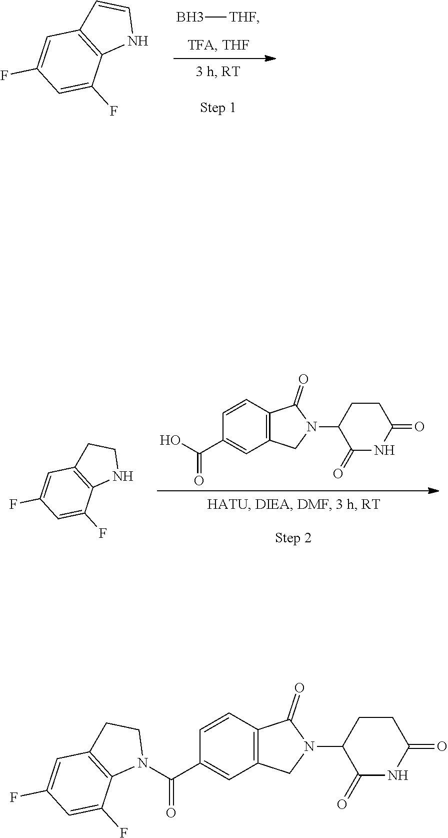

Step 1. Preparation of 5,7-difluoroindoline. A solution of 5,7-difluoro-1H-indole (500 mg, 3.265 mmol, 1 equiv) in THF (5 mL) was treated with TFA (2.5 mL) for 5 min at room temperature under nitrogen atmosphere followed by the addition of BH₃-THF (3.3 mL) dropwise at 0° C. The resulting mixture was stirred for additional 3 h at room temperature. The reaction was quenched by the addition of sat. NH₄Cl (aq.) (2 mL) at 0° C. The resulting mixture was extracted with EtOAc (4×50 mL). The combined organic layers were washed with brine (3×50 mL), dried over anhydrous Na₂SO₄. After filtration, the filtrate was concentrated under reduced pressure. The residue was purified by reverse-phase flash chromatography to afford 5,7-difluoroindoline (300 mg, 59%) as an off-white solid.

Step 2. Preparation of 3-(5-(5,7-difluoroindoline-1-carbonyl)-1-oxoisoindolin-2-yl)piperidine-2,6-dione. A solution of 5,7-difluoroindoline (64.6 mg, 0.417 mmol, 1.5 equiv), DIEA (107.6 mg, 0.834 mmol, 3 equiv) and 2-(2,6-dioxopiperidin-3-yl)-1-oxoisoindole-5-carboxylic acid (80 mg, 0.278 mmol, 1.00 equiv) in DMF (3 mL) was stirred for 3 min at room temperature. To the above mixture was added HATU (158.3 mg, 0.417 mmol, 1.5 equiv) in portions over 5 min at room temperature. The resulting mixture was stirred for additional 3 h at room temperature. The resulting mixture was diluted with water and extracted with CH₂Cl₂ (3×20 mL). The combined organic layers were washed with brine (3×30 mL), dried over anhydrous Na₂SO₄. After filtration, the filtrate was concentrated under reduced pressure. The crude product was purified by Prep-HPLC to afford 3-(5-(5,7-difluoroindoline-1-carbonyl)-1-oxoisoindolin-2-yl)piperidine-2,6-dione (13 mg, 11%) as an off-white solid. LC-MS (ES, m/z)=426.10 [M+1]⁺. ¹H NMR (400 MHz, DMSO-d₆) δ 11.02 (s, 1H), 7.93 (s, 1H), 7.87-7.77 (m, 2H), 7.15-7.06 (m, 2H), 5.16 (dd, J=13.3, 5.1 Hz, 1H), 4.58-4.41 (m, 2H), 4.13 (t, J=7.8 Hz, 2H), 3.12 (t, J=7.7 Hz, 2H), 2.93 (ddd, J=18.2, 13.6, 5.4 Hz, 1H), 2.61 (d, J=17.1 Hz, 1H), 2.49-2.42 (m, 1H), 2.39 (dd, J=13.2, 4.5 Hz, 1H).

### Example 58

### Synthesis of 2-(2,6-dioxopiperidin-3-yl)-1-oxo-N-(thiophen-3-yl)isoindoline-5-carboxamide

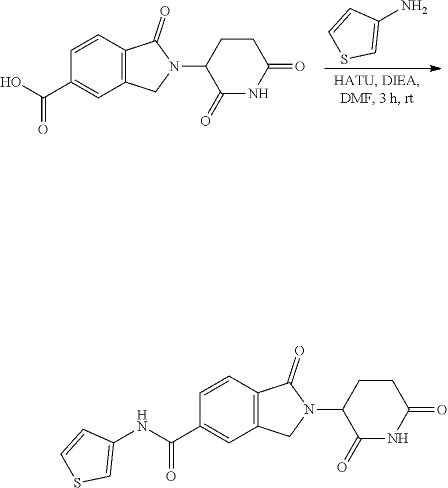

Step 1. Preparation of 2-(2,6-dioxopiperidin-3-yl)-1-oxo-N-(thiophen-3-yl)isoindoline-5-carboxamide. A solution of 2-(2,6-dioxopiperidin-3-yl)-1-oxoisoindole-5-carboxylic acid (50 mg, 0.173 mmol, 1 equiv), DIEA (67.3 mg, 0.519 mmol, 3 equiv) and thiophen-3-amine (25.8 mg, 0.259 mmol, 1.50 equiv) in DMF (1 mL) was stirred for 3 min at room temperature. To the above mixture was added HATU (98.9 mg, 0.259 mmol, 1.50 equiv) at room temperature. The resulting mixture was stirred for additional 3 h at room temperature. The resulting mixture was diluted with water and extracted with EtOAc (3×10 mL). The combined organic layers were washed with brine (3×50 mL), dried over anhydrous Na₂SO₄. After filtration, the filtrate was concentrated under reduced pressure. The residue was purified by reverse-phase flash chromatography to afford 2-(2,6-dioxopiperidin-3-yl)-1-oxo-N-(thiophen-3-yl)isoindoline-5-carboxamide (64 mg, 100%) as an off-white solid.

LC-MS (ES, m/z)=370.10 [M+1]⁺. ¹H NMR (400 MHz, DMSO-d₆) δ 11.02 (s, 1H), 10.89 (s, 1H), 8.18 (s, 1H), 8.09 (dd, J=8.0, 1.5 Hz, 1H), 7.88 (d, J=7.8 Hz, 1H), 7.78 (dd, J=3.2, 1.3 Hz, 1H), 7.51 (dd, J=5.2, 3.2 Hz, 1H), 7.33 (dd, J=5.2, 1.3 Hz, 1H), 5.16 (dd, J=13.3, 5.1 Hz, 1H), 4.56 (d, J=17.5 Hz, 1H), 4.44 (d, J=17.5 Hz, 1H), 2.93 (ddd, J=17.2, 13.6, 5.4 Hz, 1H), 2.67-2.57 (m, 1H), 2.42 (td, J=13.2, 4.5 Hz, 1H), 2.10-2.00 (m, 1H).

### Example 59

### Synthesis of 2-(2,6-dioxopiperidin-3-yl)-1-oxo-N-(thieno[3,2-b]pyridin-3-yl)isoindoline-5-carboxamide

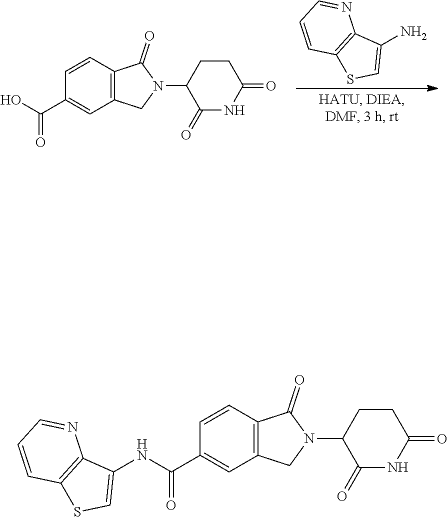 [SMILES: O=C1CCC(N2Cc3cc(C(=O)O)ccc3C2=O)C(=O)N1 ; Nc1csc2cccnc12 ; O=C1CCC(N2Cc3cc(C(=O)Nc4csc5cccnc45)ccc3C2=O)C(=O)N1]

Step 1. Preparation of 2-(2,6-dioxopiperidin-3-yl)-1-oxo-N-(thieno[3,2-b]pyridin-3-yl)isoindoline-5-carboxamide. A solution of 2-(2,6-dioxopiperidin-3-yl)-1-oxoisoindole-5-carboxylic acid (50 mg, 0.173 mmol, 1 equiv), DIEA (67.3 mg, 0.519 mmol, 3 equiv) and thieno[3,2-b]pyridin-3-amine (39.1 mg, 0.259 mmol, 1.5 equiv) in DMF (1 mL) was stirred for 3 min at room temperature. To the above mixture was added HATU (98.9 mg, 0.259 mmol, 1.5 equiv) at room temperature. The resulting mixture was stirred for additional 3 h at room temperature. The resulting mixture was diluted with water and extracted with EtOAc (3×10 mL). The combined organic layers were washed with brine (3×50 mL), dried over anhydrous Na2SO4. After filtration, the filtrate was concentrated under reduced pressure. The residue was purified by reverse-phase flash chromatography to afford 2-(2,6-dioxopiperidin-3-yl)-1-oxo-N-(thieno[3,2-b]pyridin-3-yl)isoindoline-5-carboxamide (34 mg, 47%) as an off-white solid.

LC-MS (ES, m/z)=421 [M+1]⁺. ¹H NMR (400 MHz, DMSO-d₆) δ 11.03 (s, 1H), 10.41 (s, 1H), 8.76 (dd, J=4.5, 1.4 Hz, 1H), 8.55 (dd, J=8.2, 1.4 Hz, 1H), 8.44 (s, 1H), 8.27 (d, J=1.5 Hz, 1H), 8.16 (dd, J=8.0, 1.5 Hz, 1H), 7.91 (d, J=7.9 Hz, 1H), 7.51 (dd, J=8.2, 4.5 Hz, 1H), 5.17 (dd, J=13.3, 5.1 Hz, 1H), 4.64-4.40 (m, 2H), 2.94 (ddd, J=17.3, 13.6, 5.4 Hz, 1H), 2.67-2.58 (m, 1H), 2.43 (td, J=13.1, 4.5 Hz, 1H), 2.11-2.01 (m, 1H).

### Example 60

### Synthesis of 3-(5-(5-chloro-7-fluoroindoline-1-carbonyl)-1-oxoisoindolin-2-yl)piperidine-2,6-dione

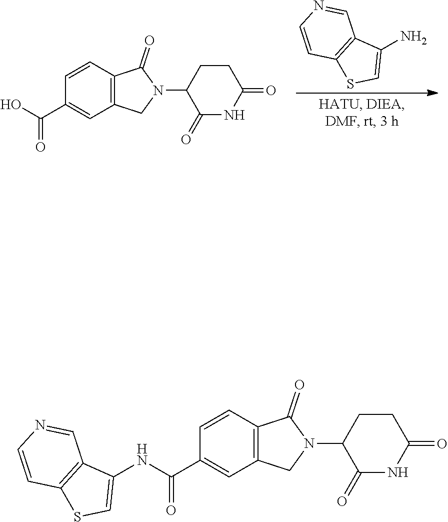 [SMILES: O=C1CCC(N2Cc3cc(C(=O)O)ccc3C2=O)C(=O)N1 ; Nc1csc2ccncc12 ; O=C1CCC(N2Cc3cc(C(=O)Nc4csc5ccncc45)ccc3C2=O)C(=O)N1]

Step 1. Preparation of 3-(5-(5-chloro-7-fluoroindoline-1-carbonyl)-1-oxoisoindolin-2-yl)piperidine-2,6-dione. To a stirred mixture of 2-(2,6-dioxopiperidin-3-yl)-1-oxoisoindole-5-carboxylic acid (100 mg, 0.347 mmol, 1 equiv) and thieno[3,2-c]pyridin-3-amine (52.1 mg, 0.347 mmol, 1 equiv) in DMF (1 mL, 12.922 mmol, 37.25 equiv) was added DIEA (134.5 mg, 1.041 mmol, 3 equiv) and HATU (197.8 mg, 0.520 mmol, 1.5 equiv) at room temperature. The resulting mixture was stirred for 3 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by Prep-HPLC to afford 3-(5-(5-chloro-7-fluoroindoline-1-carbonyl)-1-oxoisoindolin-2-yl)piperidine-2,6-dione (49 mg, 34%) as an off-white solid.

LC-MS (ES, m/z)=421.05 [M+1]⁺. ¹H NMR (400 MHz, DMSO-d₆) δ 11.04 (s, 1H), 10.88 (s, 1H), 9.43 (s, 1H), 8.49 (d, J=5.6 Hz, 1H), 8.28 (s, 1H), 8.21-8.15 (m, 2H), 8.15-8.09 (m, 1H), 7.92 (d, J=7.9 Hz, 1H), 5.18 (dd, J=13.3, 5.1 Hz, 1H), 4.59 (d, J=17.6 Hz, 1H), 4.47 (d, J=17.6 Hz, 1H), 2.94 (ddd, J=17.9, 13.6, 5.3 Hz, 1H), 2.63 (d, J=17.3 Hz, 1H), 2.44 (td, J=13.1, 4.4 Hz, 1H), 2.06 (d, J=10.3 Hz, 1H).

### Example 61

### Synthesis of 2-(2,6-dioxopiperidin-3-yl)-1-oxo-N-(thieno[2,3-c]pyridin-3-yl)isoindoline-5-carboxamide

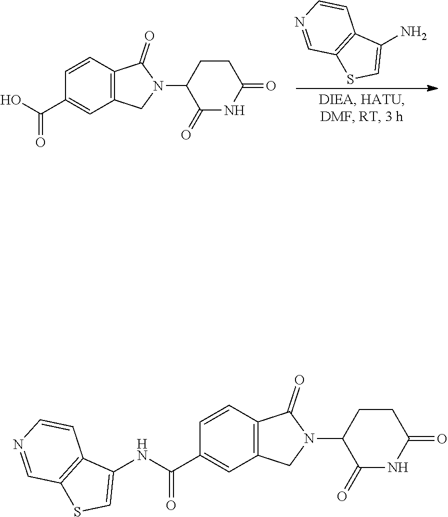 [SMILES: O=C1CCC(N2Cc3cc(C(=O)O)ccc3C2=O)C(=O)N1 ; Nc1csc2cnccc12 ; O=C1CCC(N2Cc3cc(C(=O)Nc4csc5cnccc45)ccc3C2=O)C(=O)N1]

Step 1. Preparation of 2-(2,6-dioxopiperidin-3-yl)-1-oxo-N-(thieno[2,3-c]pyridin-3-yl)isoindoline-5-carboxamide. To a stirred mixture of 2-(2,6-dioxopiperidin-3-yl)-1-oxo-3H-isoindole-5-carboxylic acid (50 mg, 0.173 mmol, 1 equiv) and thieno[2,3-c]pyridin-3-amine (26.0 mg, 0.173 mmol, 1 equiv) in DMF (1 mL, 12.922 mmol, 74.50 equiv) were added DIEA (67.2 mg, 0.519 mmol, 3 equiv) and HATU (98.9 mg, 0.259 mmol, 1.5 equiv) at room temperature. The resulting mixture was stirred for 3 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 2-(2,6-dioxopiperidin-3-yl)-1-oxo-N-(thieno[2,3-c]pyridin-3-yl)isoindoline-5-carboxamide (23 mg, 31%) as a white solid.

LC-MS (ES, m/z)=421.05 [M+1]⁺. ¹H NMR (400 MHz, DMSO-d₆) δ 11.03 (s, 1H), 10.80 (s, 1H), 9.27 (d, J=1.0 Hz, 1H), 8.53 (d, J=5.6 Hz, 1H), 8.37 (s, 1H), 8.25 (s, 1H), 8.19-8.09 (m, 2H), 7.91 (d, J=7.9 Hz, 1H), 5.18 (dd, J=13.2, 5.1 Hz, 1H), 4.59 (d, J=17.6 Hz, 1H), 4.46 (d, J=17.5 Hz, 1H), 3.00-2.88 (m, 1H), 2.63 (d, J=17.2 Hz, 1H), 2.47-2.39 (m, 1H), 2.07 (s, 1H).

### Example 62

### Synthesis of 2-(2,6-dioxopiperidin-3-yl)-1-oxo-N-(thieno[2,3-b]pyridin-3-yl)isoindoline-5-carboxamide

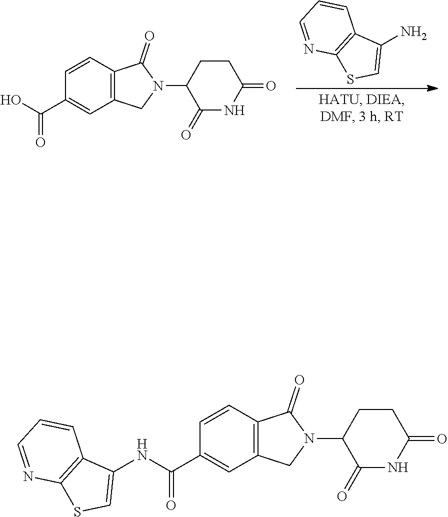 [SMILES: O=C1CCC(N2Cc3cc(C(=O)O)ccc3C2=O)C(=O)N1 ; Nc1csc2ncccc12 ; O=C1CCC(N2Cc3cc(C(=O)Nc4csc5ncccc45)ccc3C2=O)C(=O)N1]

Step 1. Preparation of 2-(2,6-dioxopiperidin-3-yl)-1-oxo-N-(thieno[2,3-b]pyridin-3-yl)isoindoline-5-carboxamide. A solution of thieno[2,3-b]pyridin-3-amine (26.1 mg, 0.173 mmol, 1 equiv), DIEA (67.3 mg, 0.519 mmol, 3 equiv) and 2-(2,6-dioxopiperidin-3-yl)-1-oxoisoindole-5-carboxylic acid (50 mg, 0.173 mmol, 1.00 equiv) in DMF (1 mL) was stirred for 3 min at room temperature. To the above mixture was added HATU (98.9 mg, 0.259 mmol, 1.5 equiv) in portions over 5 min at room temperature. The resulting mixture was stirred for additional 3 h at room temperature. The resulting mixture was diluted with water (10 mL) and extracted with EtOAc (3×50 mL). The combined organic layers were washed with brine (3×50 mL), dried over anhydrous Na₂SO₄. After filtration, the filtrate was concentrated under reduced pressure. The crude product (70 mg) was purified by Prep-HPLC to afford 2-(2,6-dioxopiperidin-3-yl)-1-oxo-N-(thieno[2,3-b]pyridin-3-yl)isoindoline-5-carboxamide (36.3 mg, 49.78%) as an off-white solid.

LC-MS (ES, m/z)=421.15 [M+1]⁺. ¹H NMR (400 MHz, DMSO-d₆) δ 11.03 (s, 1H), 10.76 (s, 1H), 8.63 (dd, J=4.6, 1.6 Hz, 1H), 8.54 (dd, J=8.1, 1.6 Hz, 1H), 8.24 (s, 1H), 8.19-8.11 (m, 2H), 7.91 (d, J=7.9 Hz, 1H), 7.51 (dd, J=8.2, 4.6 Hz, 1H), 5.18 (dd, J=13.2, 5.1 Hz, 1H), 4.63-4.42 (m, 2H), 2.94 (ddd, J=17.9, 13.6, 5.4 Hz, 1H), 2.63 (d, J=17.2 Hz, 1H), 2.44 (td, J=13.1, 4.4 Hz, 1H), 2.08 (s, 2H).

### Example 63

### Synthesis of 3-(5-(4,7-difluoroindoline-1-carbonyl)-1-oxoisoindolin-2-yl)piperidine-2,6-dione

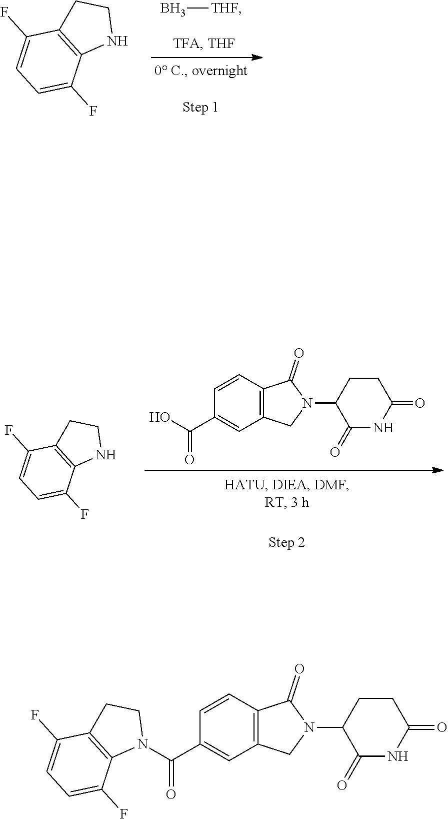

Step 1. Preparation of 4,7-difluoroindoline. A solution of 4,7-difluoro-1H-indole (400 mg, 2.612 mmol, 1 equiv) in THF (8.00 mL, 98.734 mmol, 37.80 equiv) was treated with BH₃-THF (7.84 mL, 7.836 mmol, 3 equiv) for 30 min at 0° C. under nitrogen atmosphere followed by the addition of TFA (7.84 mL, 105.551 mmol, 40.41 equiv) dropwise at 0° C. The resulting mixture was stirred for overnight at 0° C. under nitrogen atmosphere. The reaction was quenched by the addition of sat. NH₄Cl (aq.) (20 mL) at 0° C. The resulting mixture was extracted with EtOAc (3×20 mL). The combined organic layers were washed with brine (2×30 mL), dried over anhydrous Na₂SO₄. After filtration, the filtrate was concentrated under reduced pressure. The residue was purified by reverse-phase flash chromatography to afford 4,7-difluoroindoline (100 mg, 25%) as a yellow oil.

Step 2. Preparation of 3-(5-(4,7-difluoroindoline-1-carbonyl)-1-oxoisoindolin-2-yl)piperidine-2,6-dione. To a stirred mixture of 4,7-difluoro-2,3-dihydro-1H-indole (50 mg, 0.322 mmol, 1 equiv) and 2-(2,6-dioxopiperidin-3-yl)-1-oxoisoindole-5-carboxylic acid (92.9 mg, 0.322 mmol, 1 equiv) in DMF (1 mL, 12.922 mmol, 40.10 equiv) were added DIEA (124.9 mg, 0.966 mmol, 3 equiv) and HATU (183.8 mg, 0.483 mmol, 1.5 equiv) at room temperature. The resulting mixture was stirred for 3 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-(4,7-difluoroindoline-1-carbonyl)-1-oxoisoindolin-2-yl)piperidine-2,6-dione (7.7 mg, 5.5%) as a white solid.

LC-MS (ES, m/z)=426 [M+1]⁺. ¹H NMR (400 MHz, DMSO-d₆) δ 11.02 (s, 1H), 7.94 (s, 1H), 7.87-7.77 (m, 2H), 7.13 (td, J=9.4, 4.1 Hz, 1H), 7.01 (td, J=8.5, 3.1 Hz, 1H), 5.16 (dd, J=13.3, 5.1 Hz, 1H), 4.54 (d, J=17.6 Hz, 1H), 4.41 (d, J=17.6 Hz, 1H), 4.18 (t, J=7.8 Hz, 2H), 3.15 (t, J=7.8 Hz, 2H), 2.99-2.88 (m, 1H), 2.61 (d, J=16.9 Hz, 1H), 2.42 (dd, J=13.1, 4.6 Hz, 1H), 2.28 (t, J=7.3 Hz, 1H), 2.08-2.00 (m, 1H).

### Example 64

### Synthesis of 3-(5-(7-fluoro-5-methoxyindoline-1-carbonyl)-1-oxoisoindolin-2-yl)piperidine-2,6-dione

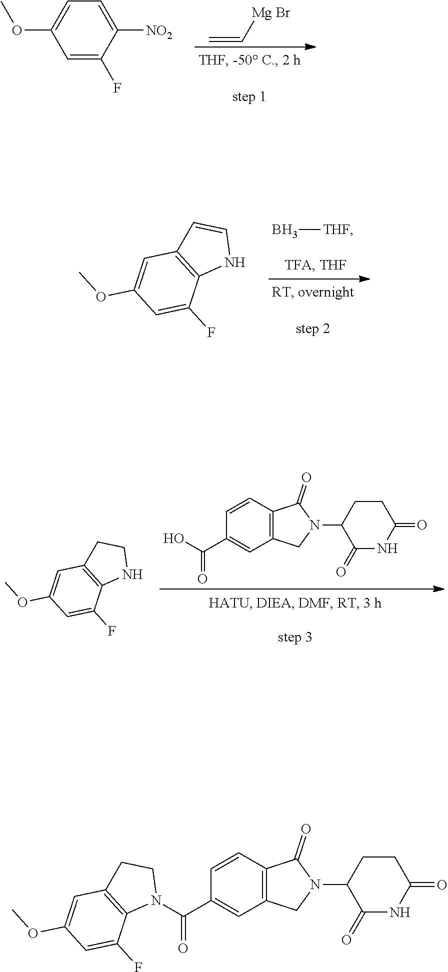

Step 1. Preparation of 7-fluoro-5-methoxy-1H-indole. To a stirred mixture of 2-fluoro-4-methoxy-1-nitrobenzene (5 g, 29.218 mmol, 1 equiv) in THF (99.99 mL, 1234.168 mmol, 42.24 equiv) was added bromo(ethenyl)magnesium (87.65 mL, 87.654 mmol, 3 equiv) dropwise at −50° C. under nitrogen atmosphere. The resulting mixture was stirred for 2 h at −50° C. under nitrogen atmosphere. The reaction was quenched by the addition of sat. NH₄Cl (aq.) (20 mL) at 0° C. The mixture neutralized to pH 7 with HCl (aq.). The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by reverse-phase flash chromatography to afford 7-fluoro-5-methoxy-1H-indole (500 mg, 10%) as a yellow oil.

Step 2. Preparation of 7-fluoro-5-methoxyindoline. A solution of 7-fluoro-5-methoxy-1H-indole (500 mg, 3.027 mmol, 1 equiv) in THF (5.00 mL, 61.721 mmol, 20.39 equiv) was treated with BH₃-THF (18.16 mL, 18.162 mmol, 6 equiv) for 30 min at 0° C. under nitrogen atmosphere followed by the addition of TFA (15.00 mL, 201.931 mmol, 66.71 equiv) dropwise at 0° C. The resulting mixture was stirred for overnight at room temperature under nitrogen atmosphere. The reaction was quenched by the addition of Water (10 mL) at 0° C. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by reverse-phase flash chromatography to afford 7-fluoro-5-methoxyindoline (100 mg, 20%) as a yellow oil.

Step 3. Preparation of 3 3-(5-(7-fluoro-5-methoxyindoline-1-carbonyl)-1-oxoisoindolin-2-yl)piperidine-2,6-dione. To a stirred mixture of 7-fluoro-5-methoxy-2,3-dihydro-1H-indole (50 mg, 0.299 mmol, 1 equiv) and 2-(2,6-dioxopiperidin-3-yl)-1-oxoisoindole-5-carboxylic acid (86.2 mg, 0.299 mmol, 1 equiv) in DMF (1 mL, 12.922 mmol, 43.21 equiv) were added DIEA (115.9 mg, 0.897 mmol, 3 equiv) and HATU (170.5 mg, 0.449 mmol, 1.5 equiv) at room temperature. The resulting mixture was stirred for 3 h at room temperature. The resulting mixture was extracted with EtOAc (3×10 mL). The combined organic layers were washed with brine (2×5 mL), dried over anhydrous Na₂SO₄. After filtration, the filtrate was concentrated under reduced pressure. The residue was purified by reverse-phase flash chromatography to afford 3-(5-(7-fluoro-5-methoxyindoline-1-carbonyl)-1-oxoisoindolin-2-yl)piperidine-2,6-dione (14.5 mg, 11%) as an off-white solid.

LC-MS (ES, m/z)=438.15 [M+1]⁺. ¹H NMR (400 MHz, DMSO-d₆) δ 11.02 (s, 1H), 7.90 (s, 1H), 7.85-7.74 (m, 2H), 6.80 (dd, J=2.3, 1.2 Hz, 1H), 6.68 (d, J=12.8 Hz, 1H), 5.15 (dd, J=13.3, 5.1 Hz, 1H), 4.53 (d, J=17.7 Hz, 1H), 4.40 (d, J=17.5 Hz, 1H), 4.09 (t, J=7.7 Hz, 2H), 3.75 (s, 3H), 3.07 (t, J=7.6 Hz, 2H), 2.97-2.86 (m, 1H), 2.61 (d, J=17.0 Hz, 1H), 2.40 (td, J=13.1, 4.4 Hz, 1H), 2.10-2.00 (m, 1H).

### Example 65

### Synthesis of 3-(5-(4,5-difluoroindoline-1-carbonyl)-1-oxoisoindolin-2-yl)piperidine-2,6-dione

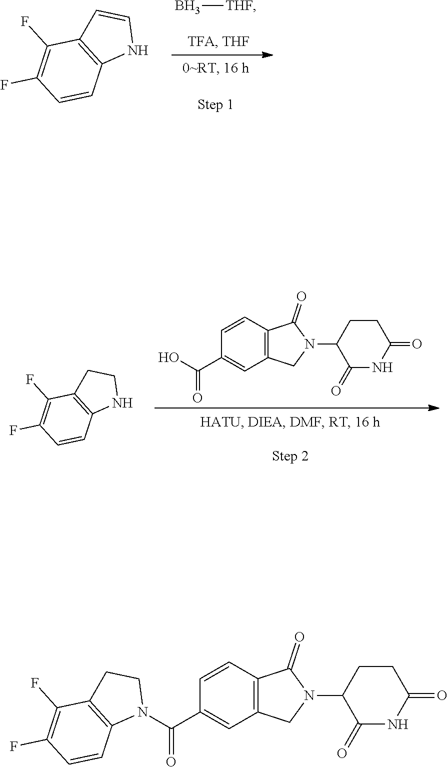

Step 1. Preparation of 4,5-difluoroindoline. To a stirred solution of 4,5-difluoro-1H-indole (500 mg, 3.265 mmol, 1 equiv) in THF (5 mL) was added BH₃-THF (1M, 3.3 ml) dropwise at 0° C. under nitrogen atmosphere. The resulting mixture was stirred for 30 min at 0° C. under nitrogen atmosphere. To the above mixture was added TFA (2.5 mL) dropwise over 3 min at 0° C. The resulting mixture was stirred for additional overnight at room temperature. The reaction was quenched with MeOH at 0° C. The resulting mixture was concentrated under reduced pressure. The residue was purified by reverse-phase flash chromatography to afford 4,5-difluoroindoline (500 mg, crude) as a brown oil.

Step 2. Preparation of 3-(5-(4,5-difluoroindoline-1-carbonyl)-1-oxoisoindolin-2-yl)piperidine-2,6-dione. To a stirred solution of 4,5-difluoroindoline (400 mg, 2.578 mmol, 5 equiv), 2-(2,6-dioxopiperidin-3-yl)-1-oxoisoindole-5-carboxylic acid (148.6 mg, 0.516 mmol, 1 equiv) and DIEA (333.2 mg, 2.578 mmol, 5 equiv) in DMF (4 mL) was added HATU (294.0 mg, 0.773 mmol, 1.5 equiv) in portions at room temperature under nitrogen atmosphere. The resulting mixture was stirred for overnight at room temperature under nitrogen atmosphere. The resulting mixture was diluted with water (50 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product (100 mg) was purified by Prep-HPLC to afford 3-(5-(4,5-difluoroindoline-1-carbonyl)-1-oxoisoindolin-2-yl)piperidine-2,6-dione (18 mg, 8%) as an off-white solid.

LC-MS (ES, m/z)=424.15 [M−1]⁻. ¹H NMR (400 MHz, DMSO-d₆) δ 11.02 (s, 1H), 7.98-7.80 (m, 3H), 7.72 (d, J=7.9 Hz, 1H), 7.29 (s, 1H), 5.16 (dd, J=13.3, 5.1 Hz, 1H), 4.58-4.37 (m, 2H), 4.10 (t, J=8.2 Hz, 2H), 3.18 (t, J=8.3 Hz, 2H), 2.93 (ddd, J=17.1, 13.6, 5.4 Hz, 1H), 2.67-2.57 (m, 1H), 2.43 (qd, J=13.1, 4.4 Hz, 1H), 2.04 (ddd, J=15.7, 7.1, 4.4 Hz, 1H).

### Example 66

### Synthesis of 3-(5-(5-chloro-4-fluoroindoline-1-carbonyl)-1-oxoisoindolin-2-yl)piperidine-2,6-dione

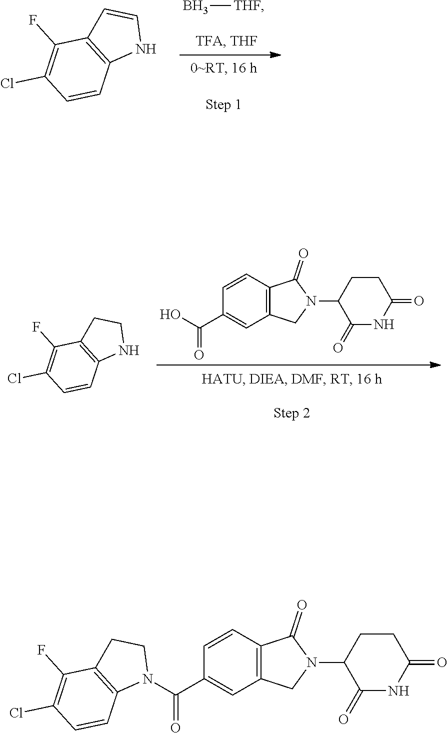

Step 1. Preparation of 5-chloro-4-fluoroindoline. To a stirred solution of 5-chloro-4-fluoro-1H-indole (500 mg, 2.948 mmol, 1 equiv) in THF (5 mL) was added BH₃-THF 1M 3.3 mL) dropwise at 0° C. under nitrogen atmosphere. The resulting mixture was stirred for 30 min at 0° C. under nitrogen atmosphere. To the above mixture was added TFA (2.5 mL) dropwise over 3 min at 0° C. The resulting mixture was stirred for additional overnight at room temperature. The reaction was quenched by the addition of MeOH (5 mL) at 0° C. The resulting mixture was concentrated under reduced pressure. The residue was purified by reverse-phase flash chromatography to afford 5-chloro-4-fluoroindoline (500 mg, crude) as a brown oil.

Step 2. Preparation of 3-(5-(5-chloro-4-fluoroindoline-1-carbonyl)-1-oxoisoindolin-2-yl)piperidine-2,6-dione. To a stirred solution of 5-chloro-4-fluoroindoline (200 mg, 1.166 mmol, 4 equiv), 2-(2,6-dioxopiperidin-3-yl)-1-oxoisoindole-5-carboxylic acid (83.9 mg, 0.291 mmol, 1 equiv) and DIEA (188.3 mg, 1.458 mmol, 5 equiv) in DMF (4 mL) was added HATU (166.1 mg, 0.437 mmol, 1.5 equiv) in portions at room temperature under nitrogen atmosphere. The resulting mixture was stirred for overnight at room temperature under nitrogen atmosphere. The resulting mixture was diluted with water (50 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product (100 mg) was purified by Prep-HPLC to afford 3-(5-(5-chloro-4-fluoroindoline-1-carbonyl)-1-oxoisoindolin-2-yl)piperidine-2,6-dione (16 mg, 12%) as an off-white solid. LC-MS (ES, m/z)=440 [M−1]⁻. ¹H NMR (400 MHz, DMSO-d₆) δ 11.02 (s, 1H), 8.10-7.80 (m, 3H), 7.73 (d, J=8.0 Hz, 1H), 7.43 (s, 1H), 5.16 (dd, J=13.3, 5.1 Hz, 1H), 4.57-4.38 (m, 2H), 4.10 (t, J=8.2 Hz, 2H), 3.17 (t, J=8.3 Hz, 2H), 2.93 (ddd, J=18.0, 13.6, 5.3 Hz, 1H), 2.66-2.57 (m, 1H), 2.41 (td, J=13.3, 4.5 Hz, 1H), 2.10-1.97 (m, 1H).

### Example 67

### Synthesis of 2-(2,6-dioxopiperidin-3-yl)-N-(4-fluorobenzo[b]thiophen-3-yl)-1-oxoisoindoline-5-carboxamide

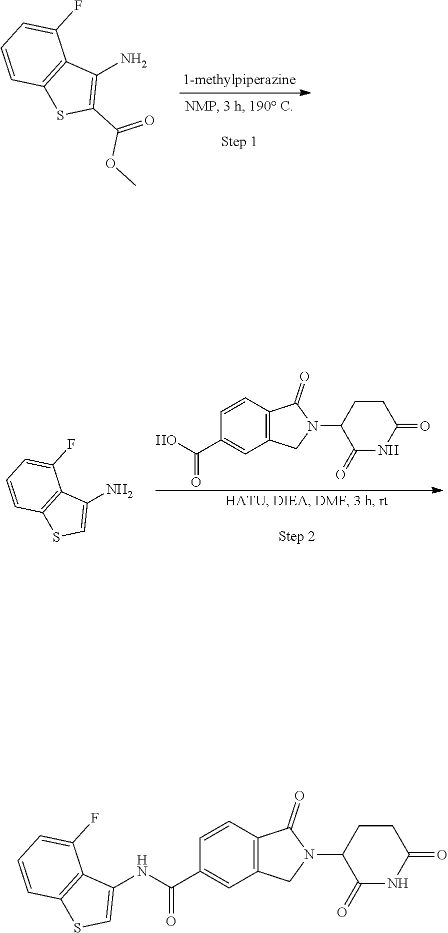 [SMILES: COC(=O)c1sc2cccc(F)c2c1N ; Nc1csc2cccc(F)c12 ; O=C1CCC(N2Cc3cc(C(=O)O)ccc3C2=O)C(=O)N1 ; O=C1CCC(N2Cc3cc(C(=O)Nc4csc5cccc(F)c45)ccc3C2=O)C(=O)N1]

Step 1. Preparation of 4-fluorobenzo[b]thiophen-3-amine. A solution of methyl 3-amino-4-fluoro-1-benzothiophene-2-carboxylate (200 mg, 0.888 mmol, 1 equiv) and 1-methylpiperazine (88.9 mg, 0.888 mmol, 1 equiv) in NMP (1.5 mL) was stirred for 3 h at 190° C. under nitrogen atmosphere. The reaction was quenched with brine at 0° C. The resulting mixture was extracted with EtOAc (3×80 mL). The combined organic layers were washed with brine (3×80 mL), dried over anhydrous Na₂SO₄. After filtration, the filtrate was concentrated under reduced pressure. The residue was purified by reverse-phase flash chromatography to afford 4-fluorobenzo[b]thiophen-3-amine (90 mg, 61%) as an off-white solid.

Step 2. Preparation of 2-(2,6-dioxopiperidin-3-yl)-N-(4-fluorobenzo[b]thiophen-3-yl)-1-oxoisoindoline-5-carboxamide. A solution of 4-fluorobenzo[b]thiophen-3-amine (69.6 mg, 0.417 mmol, 1.5 equiv), DIEA (107.6 mg, 0.834 mmol, 3 equiv) and 2-(2,6-dioxopiperidin-3-yl)-1-oxoisoindole-5-carboxylic acid (80 mg, 0.278 mmol, 1.00 equiv) in DMF (1 mL) was stirred for 3 min at room temperature. To the above mixture was added HATU (158.3 mg, 0.417 mmol, 1.5 equiv) in portions over 5 min at room temperature. The resulting mixture was stirred for additional 3 h at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reverse-phase flash chromatography to afford 2-(2,6-dioxopiperidin-3-yl)-N-(4-fluorobenzo[b]thiophen-3-yl)-1-oxoisoindoline-5-carboxamide (18 mg, 15%) as an off-white solid.

LC-MS (ES, m/z)=438.05 [M+1]⁺. ¹H NMR (400 MHz, DMSO-d₆) δ 11.03 (s, 1H), 10.37 (s, 1H), 8.20 (s, 1H), 8.11 (dd, J=7.9, 1.5 Hz, 1H), 7.93-7.82 (m, 3H), 7.43 (td, J=8.0, 4.9 Hz, 1H), 7.20 (dd, J=11.8, 7.9 Hz, 1H), 5.17 (dd, J=13.3, 5.1 Hz, 1H), 4.62-4.43 (m, 2H), 2.94 (ddd, J=17.9, 13.6, 5.4 Hz, 1H), 2.62 (d, J=17.4 Hz, 1H), 2.43 (td, J=13.2, 4.4 Hz, 1H), 2.09-2.02 (m, 1H).

### Example 68

### Synthesis of 2-(2,6-dioxopiperidin-3-yl)-N-(5-fluorobenzo[b]thiophen-3-yl)-1-oxoisoindoline-5-carboxamide

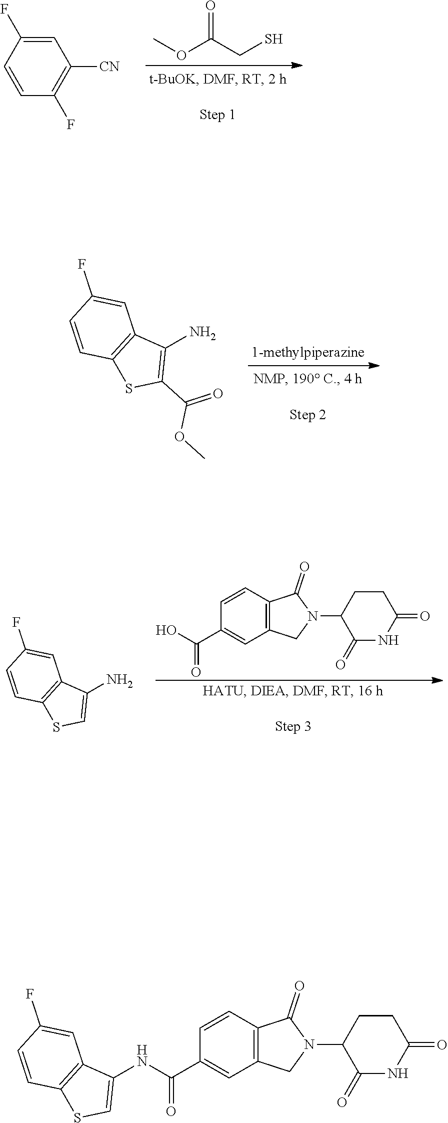

Step 1. Preparation of methyl 3-amino-5-fluorobenzo[b]thiophene-2-carboxylate. To a stirred solution of 2,5-difluorobenzonitrile (1 g, 7.189 mmol, 1 equiv) and methyl thioglycolate (1.14 g, 10.784 mmol, 1.5 equiv) in DMF (10 mL) was added t-BuOK (2.02 g, 17.973 mmol, 2.5 equiv) in portions at room temperature under nitrogen atmosphere. The resulting mixture was stirred for 2 h at room temperature under nitrogen atmosphere. The reaction was quenched with Water/Ice at 0° C. The resulting mixture was diluted with water (100 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by reverse-phase flash chromatography to afford methyl 3-amino-5-fluorobenzo[b]thiophene-2-carboxylate (1 g, 62%) as an off-white solid. Step 2. Preparation of 5-fluorobenzo[b]thiophen-3-amine. To a stirred solution of methyl 3-amino-5-fluorobenzo[b]thiophene-2-carboxylate (1 g, 4.440 mmol, 1 equiv) in NNW (10 mL) was added 1-methylpiperazine (0.44 g, 4.440 mmol, 1 equiv) at room temperature under nitrogen atmosphere. The resulting mixture was stirred for 4 h at 190° C. under nitrogen atmosphere. The mixture was allowed to cool down to room temperature. The resulting mixture was diluted with water (100 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by reverse-phase flash chromatography to afford 5-fluorobenzo[b]thiophen-3-amine (700 mg, 94%) as an off-white solid.

Step 3. Preparation of 2-(2,6-dioxopiperidin-3-yl)-N-(5-fluorobenzo[b]thiophen-3-yl)-1-oxoisoindoline-5-carboxamide. To a stirred solution of 5-fluorobenzo[b]thiophen-3-amine (60 mg, 0.359 mmol, 1 equiv), 2-(2,6-dioxopiperidin-3-yl)-1-oxoisoindole-5-carboxylic acid (103.4 mg, 0.359 mmol, 1 equiv) and DIEA (139.1 mg, 1.077 mmol, 3 equiv) in DMF (2 mL) was added HATU (204.6 mg, 0.538 mmol, 1.5 equiv) in portions at room temperature under nitrogen atmosphere. The resulting mixture was stirred for overnight at room temperature under nitrogen atmosphere. The resulting mixture was diluted with water (50 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by reverse-phase flash chromatography to afford 2-(2,6-dioxopiperidin-3-yl)-N-(5-fluorobenzo[b]thiophen-3-yl)-1-oxoisoindoline-5-carboxamide (31.1 mg, 19.81%) as an off-white solid.

LC-MS (ES, m/z)=436.10 [M−1]⁻. ¹H NMR (400 MHz, DMSO-d₆) δ 11.03 (s, 1H), 10.59 (s, 1H), 8.25 (s, 1H), 8.19 (s, 1H), 8.15 (dd, J=8.0, 1.5 Hz, 1H), 8.03 (ddd, J=12.8, 9.6, 3.8 Hz, 2H), 7.90 (d, J=7.9 Hz, 1H), 7.32 (td, J=8.8, 2.5 Hz, 1H), 5.18 (dd, J=13.3, 5.1 Hz, 1H), 4.63-4.41 (m, 2H), 2.94 (ddd, J=17.0, 13.6, 5.3 Hz, 1H), 2.63 (ddd, J=17.4, 4.6, 2.3 Hz, 1H), 2.44 (td, J=13.2, 4.5 Hz, 1H), 2.06 (dtd, J=10.8, 5.8, 5.3, 2.8 Hz, 1H).

### Example 69

### Synthesis of 2-(2,6-dioxopiperidin-3-yl)-N-(6-fluorobenzo[b]thiophen-3-yl)-1-oxoisoindoline-5-carboxamide

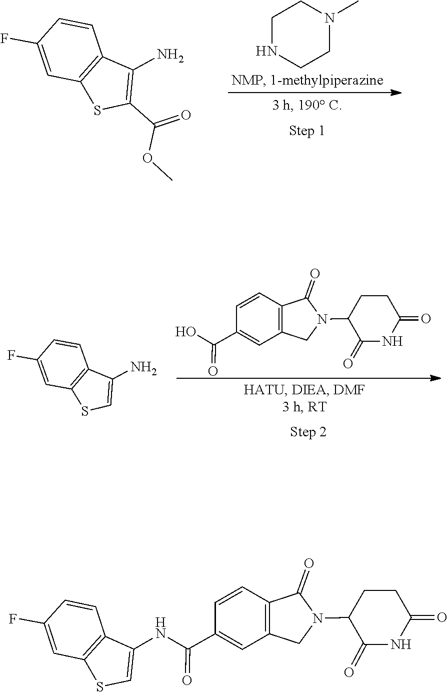

Step 1. Preparation of 6-fluorobenzo[b]thiophen-3-amine. To a stirred mixture of methyl 3-amino-6-fluoro-1-benzothiophene-2-carboxylate (500 mg, 2.220 mmol, 1 equiv) in NMP (10.00 mL, 103.696 mmol, 46.71 equiv) were added piperazine, 1-methyl-(222.4 mg, 2.220 mmol, 1 equiv) at room temperature under nitrogen atmosphere. The resulting mixture was stirred for 3 h at 190° C. under nitrogen atmosphere. The reaction was quenched with Water/Ice at 0° C. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by reverse-phase flash chromatography to afford 6-fluorobenzo[b]thiophen-3-amine (100 mg, 27%) as a yellow oil.

Step 2. Preparation of 2-(2,6-dioxopiperidin-3-yl)-N-(6-fluorobenzo[b]thiophen-3-yl)-1-oxoisoindoline-5-carboxamide. To a stirred mixture of 6-fluoro-1-benzothiophen-3-amine (58.0 mg, 0.347 mmol, 1 equiv) and 2-(2,6-dioxopiperidin-3-yl)-1-oxo-3H-isoindole-5-carboxylic acid (100 mg, 0.347 mmol, 1.00 equiv) in DNW (2 mL, 25.843 mmol, 74.50 equiv) were added DIEA (134.5 mg, 1.041 mmol, 3 equiv) and HATU (197.8 mg, 0.520 mmol, 1.5 equiv) at room temperature. The resulting mixture was stirred for 3 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 2-(2,6-dioxopiperidin-3-yl)-N-(6-fluorobenzo[b]thiophen-3-yl)-1-oxoisoindoline-5-carboxamide (8.1 mg, 5%) as an off-white solid.

LC-MS (ES, m/z)=436.10 [M−1]⁻. ¹H NMR (400 MHz, DMSO-d₆) δ 11.03 (s, 1H), 10.64 (s, 1H), 8.24 (s, 1H), 8.20-8.11 (m, 2H), 8.02 (s, 1H), 7.97-7.87 (m, 2H), 7.34 (td, J=9.0, 2.5 Hz, 1H), 5.18 (dd, J=13.3, 5.1 Hz, 1H), 4.58 (d, J=17.6 Hz, 1H), 4.46 (d, J=17.5 Hz, 1H), 2.99-2.91 (m, 1H), 2.63 (d, J=17.1 Hz, 1H), 2.45-2.39 (m, 1H), 2.07-2.03 (m, 1H).

### Example 70

### Synthesis of 2-(2,6-dioxopiperidin-3-yl)-N-(7-fluorobenzo[b]thiophen-3-yl)-1-oxoisoindoline-5-carboxamide

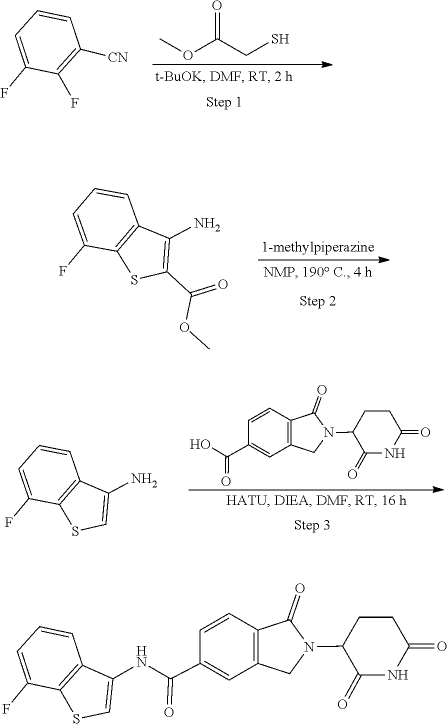

Step 1. Preparation of methyl 3-amino-7-fluorobenzo[b]thiophene-2-carboxylate. To a PUP-stirred solution of 2,3-difluorobenzonitrile (1 g, 7.189 mmol, 1 equiv) and methyl thioglycolate (1.14 g, 10.784 mmol, 1.5 equiv) in DMF (10 mL) was added t-BuOK (2.02 g, 17.973 mmol, 2.5 equiv) in portions at room temperature under nitrogen atmosphere. The resulting mixture was stirred for 2 h at room temperature under nitrogen atmosphere. The reaction was quenched with Water/Ice at 0° C. The resulting mixture was diluted with water (100 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by reverse-phase flash chromatography to afford methyl 3-amino-7-fluorobenzo[b]thiophene-2-carboxylate (600 mg, 37%) as an off-white solid.

Step 2. Preparation of 7-fluorobenzo[b]thiophen-3-amine. To a stirred solution of methyl 3-amino-7-fluorobenzo[b]thiophene-2-carboxylate (800 mg, 3.552 mmol, 1 equiv) in NMP (8 mL) was added 1-methylpiperazine (355.7 mg, 3.552 mmol, 1 equiv) at room temperature under nitrogen atmosphere. The resulting mixture was stirred for 4 h at 190° C. under nitrogen atmosphere. The mixture was allowed to cool down to room temperature. The resulting mixture was diluted with water (100 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by reverse-phase flash chromatography to afford 7-fluorobenzo[b]thiophen-3-amine (500 mg, 84%) as an off-white solid.

Step 3. Preparation of 2-(2,6-dioxopiperidin-3-yl)-N-(7-fluorobenzo[b]thiophen-3-yl)-1-oxoisoindoline-5-carboxamide. To a stirred solution of 7-fluorobenzo[b]thiophen-3-amine (60 mg, 0.359 mmol, 1 equiv), 2-(2,6-dioxopiperidin-3-yl)-1-oxoisoindole-5-carboxylic acid (103.4 mg, 0.359 mmol, 1 equiv) and DIEA (139.1 mg, 1.077 mmol, 3 equiv) in DMF (2 mL) was added HATU (204.6 mg, 0.538 mmol, 1.5 equiv) in portions at room temperature under nitrogen atmosphere. The resulting mixture was stirred for overnight at room temperature under nitrogen atmosphere. The resulting mixture was diluted with water (50 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product was purified by Prep-HPLC to afford 2-(2,6-dioxopiperidin-3-yl)-N-(7-fluorobenzo[b]thiophen-3-yl)-1-oxoisoindoline-5-carboxamide (13 mg, 8%) as an off-white solid.

LC-MS (ES, m/z)=436.05 [M−1]⁻. ¹H NMR (400 MHz, DMSO-d₆) δ 11.02 (s, 1H), 10.72 (s, 1H), 8.24 (s, 1H), 8.19-8.11 (m, 2H), 8.02 (d, J=8.1 Hz, 1H), 7.90 (d, J=7.9 Hz, 1H), 7.49 (td, J=8.0, 5.2 Hz, 1H), 7.32 (dd, J=10.2, 7.8 Hz, 1H), 5.18 (dd, J=13.3, 5.1 Hz, 1H), 4.63-4.41 (m, 2H), 2.94 (ddd, J=17.2, 13.6, 5.4 Hz, 1H), 2.63 (ddd, J=17.3, 4.4, 2.3 Hz, 1H), 2.44 (td, J=13.2, 4.5 Hz, 1H), 2.06 (ddq, J=10.6, 5.3, 2.6, 2.1 Hz, 1H).

### Example 71

### Synthesis of 2-(2,6-dioxopiperidin-3-yl)-N-(3-fluorobicyclo[1.1.1]pentan-1-yl oxoisoindoline-5-carboxamide

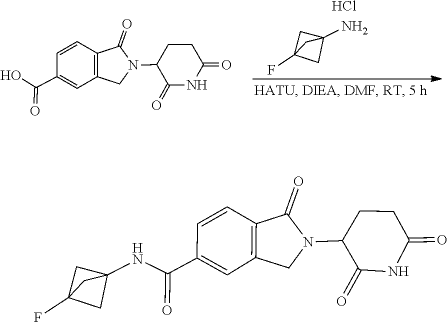 [SMILES: Cl ; O=C1CCC(N2Cc3cc(C(=O)O)ccc3C2=O)C(=O)N1 ; NC12CC(F)(C1)C2 ; O=C1CCC(N2Cc3cc(C(=O)NC45CC(F)(C4)C5)ccc3C2=O)C(=O)N1]

Step 1. Preparation of 2-(2,6-dioxopiperidin-3-yl)-N-(3-fluorobicyclo[1.1.1]pentan-1-yl)-1-oxoisoindoline-5-carboxamide. To a stirred solution of 3-fluorobicyclo[1.1.1]pentan-1-amine hydrochloride (50 mg, 0.363 mmol, 1 equiv), 2-(2,6-dioxopiperidin-3-yl)-1-oxoisoindole-5-carboxylic acid (104.8 mg, 0.363 mmol, 1 equiv) and DIEA (140.9 mg, 1.089 mmol, 3 equiv) in DMF (2 mL) was added HATU (207.3 mg, 0.544 mmol, 1.5 equiv) in portions at room temperature under nitrogen atmosphere. The resulting mixture was stirred for 5 h at room temperature under nitrogen atmosphere. The resulting mixture was diluted with water (50 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by reverse-phase flash chromatography to afford 2-(2,6-dioxopiperidin-3-yl)-N-(3-fluorobicyclo[1.1.1]pentan-1-yl)-1-oxoisoindoline-5-carboxamide (16 mg, 12%) as an off-white solid.

LC-MS (ES, m/z)=370.15 [M−1]⁻. ¹H NMR (400 MHz, DMSO-d₆) δ 11.01 (s, 1H), 9.33 (s, 1H), 8.05 (s, 1H), 7.96 (dd, J=8.0, 1.5 Hz, 1H), 7.81 (d, J=7.9 Hz, 1H), 5.14 (dd, J=13.3, 5.1 Hz, 1H), 4.55-4.36 (m, 2H), 2.92 (ddd, J=18.4, 13.7, 5.3 Hz, 1H), 2.61 (d, J=17.2 Hz, 1H), 2.43 (d, J=2.2 Hz, 7H), 2.08-1.97 (m, 1H).

### Example 72

### Synthesis of 2-(2,6-dioxopiperidin-3-yl)-N-(4-fluorobicyclo[2.2.2]octan-1-yl)-1-oxoisoindoline-5-carboxamide

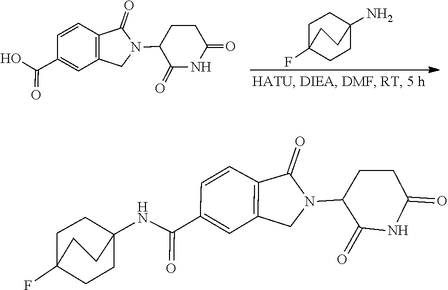 [SMILES: O=C1CCC(N2Cc3cc(C(=O)O)ccc3C2=O)C(=O)N1 ; NC12CCC(F)(CC1)CC2 ; O=C1CCC(N2Cc3cc(C(=O)NC45CCC(F)(CC4)CC5)ccc3C2=O)C(=O)N1]

Step 1. Preparation of 2-(2,6-dioxopiperidin-3-yl)-N-(4-fluorobicyclo[2.2.2]octan-1-yl)-1-oxoisoindoline-5-carboxamide. To a stirred solution of 4-fluorobicyclo[2.2.2]octan-1-amine (50 mg, 0.349 mmol, 1 equiv), 2-(2,6-dioxopiperidin-3-yl)-1-oxoisoindole-5-carboxylic acid (100.6 mg, 0.349 mmol, 1 equiv) and DIEA (135.4 mg, 1.047 mmol, 3 equiv) in DMF (2 mL) was added HATU (199.1 mg, 0.523 mmol, 1.5 equiv) in portions at room temperature under nitrogen atmosphere. The resulting mixture was stirred for 5 h at room temperature under nitrogen atmosphere. The resulting mixture was diluted with water (20 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product was purified by Prep-HPLC to afford 2-(2,6-dioxopiperidin-3-yl)-N-(4-fluorobicyclo[2.2.2]octan-1-yl)-1-oxoisoindoline-5-carboxamide (22 mg, 15%) as an off-white solid.

LC-MS (ES, m/z)=414.15 [M+1]⁺. ¹H NMR (400 MHz, DMSO-d₆) δ 11.00 (s, 1H), 7.95 (d, J=8.6 Hz, 2H), 7.88 (dd, J=7.9, 1.5 Hz, 1H), 7.76 (d, J=7.9 Hz, 1H), 5.13 (dd, J=13.3, 5.1 Hz, 1H), 4.53-4.32 (m, 2H), 2.92 (ddd, J=17.2, 13.6, 5.4 Hz, 1H), 2.65-2.56 (m, 1H), 2.42 (qd, J=13.2, 4.4 Hz, 1H), 2.15 (dd, J=10.6, 5.6 Hz, 5H), 2.02 (dtd, J=12.7, 5.4, 2.2 Hz, 1H), 1.87 (dt, J=11.8, 5.7 Hz, 6H).

### Example 73

### Synthesis of 3-(5-(2,3-dihydro-1H-pyrrolo[2,3-c]pyridine-1-carbonyl)-1-oxoisoindolin-2-yl)piperidine-2,6-dione

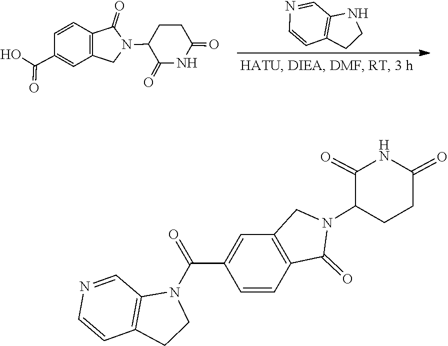 [SMILES: O=C1CCC(N2Cc3cc(C(=O)O)ccc3C2=O)C(=O)N1 ; c1cc2c(cn1)NCC2 ; O=C1CCC(N2Cc3cc(C(=O)N4CCc5ccncc54)ccc3C2=O)C(=O)N1]

Step 1. Preparation of 3-(5-(2,3-dihydro-1H-pyrrolo[2,3-c]pyridine-1-carbonyl)-1-oxoisoindolin-2-yl)piperidine-2,6-dione. A solution of 1H,2H,3H-pyrrolo[2,3-c]pyridine (200 mg, 1.665 mmol, 1 equiv) and DIEA (322.7 mg, 2.498 mmol, 1.5 equiv) in DMF (8 mL) was stirred for 5 min at room temperature. To the above mixture was added 2-(2,6-dioxopiperidin-3-yl)-1-oxoisoindole-5-carboxylic acid (575.8 mg, 1.998 mmol, 1.2 equiv) and HATU (949.4 mg, 2.498 mmol, 1.5 equiv) in portions 5 min at room temperature. The resulting mixture was stirred for additional 3 h at room temperature. The resulting mixture was filtered. the filter cake was washed with acetonitrile (3×7 mL). The filtrate was concentrated under reduced pressure. The resulting mixture was concentrated under reduced pressure. The filtrate (5 mL) was purified by Prep-HPLC to afford 3-(5-(2,3-dihydro-1H-pyrrolo[2,3-c]pyridine-1-carbonyl)-1-oxoisoindolin-2-yl)piperidine-2,6-dione (25 mg, 4%) as an off-white solid.

LC-MS (ES, m/z)=391 [M+1]⁺. ¹H NMR (400 MHz, DMSO-d₆) δ 11.03 (s, 1H), 9.25 (s, 1H), 8.45 (s, 1H), 7.92-7.84 (m, 2H), 7.81-7.74 (m, 1H), 7.69 (s, 1H), 5.16 (dd, J=13.3, 5.1 Hz, 1H), 4.55 (d, J=17.7 Hz, 1H), 4.42 (d, J=17.7 Hz, 1H), 4.12 (t, J=8.4 Hz, 3H), 3.31 (t, J=8.4 Hz, 3H), 2.93 (ddd, J=18.5, 13.6, 5.4 Hz, 1H), 2.62 (d, J=17.2 Hz, 1H), 2.46-2.37 (m, 1H), 2.04 (dd, J=9.9, 4.5 Hz, 1H).

### Example 74

### Synthesis of 3-(5-(4-fluoroindoline-1-carbonyl)-1-oxoisoindolin-2-yl)piperidine-2,6-dione

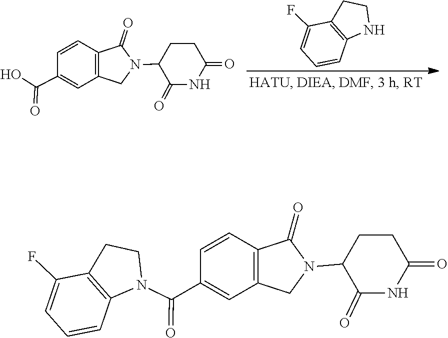 [SMILES: O=C1CCC(N2Cc3cc(C(=O)O)ccc3C2=O)C(=O)N1 ; Fc1cccc2c1CCN2 ; O=C1CCC(N2Cc3cc(C(=O)N4CCc5c(F)cccc54)ccc3C2=O)C(=O)N1]

Step 1. Preparation of 3-(5-(4-fluoroindoline-1-carbonyl)-1-oxoisoindolin-2-yl)piperidine-2,6-dione. A solution of 2-(2,6-dioxopiperidin-3-yl)-1-oxoisoindole-5-carboxylic acid (200 mg, 0.694 mmol, 1 equiv) and DIEA (269.0 mg, 2.082 mmol, 3 equiv) in DMF (3 mL) was stirred for 5 min at room temperature. To the above mixture was added 4-fluoro-2,3-dihydro-1H-indole (114.2 mg, 0.833 mmol, 1.2 equiv) and HATU (395.7 mg, 1.041 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The crude product was purified by Prep-HPLC to afford 3-(5-(4-fluoroindoline-1-carbonyl)-1-oxoisoindolin-2-yl)piperidine-2,6-dione (44 mg, 16%) as an light pink solid.

LC-MS (ES, m/z)=408 [M+1]⁺. ¹H NMR (400 MHz, DMSO-d₆) δ 11.03 (s, 1H), 7.87-7.81 (m, 3H), 7.72 (d, J=7.9 Hz, 1H), 7.27 (s, 1H), 6.93 (d, J=9.0 Hz, 1H), 5.16 (dd, J=13.3, 5.1 Hz, 1H), 4.58-4.33 (m, 2H), 4.08 (t, J=8.2 Hz, 2H), 3.12 (t, J=8.2 Hz, 2H), 2.93 (ddd, J=18.3, 13.6, 5.3 Hz, 1H), 2.61 (d, J=17.4 Hz, 2H), 2.49-2.35 (m, 1H), 2.04 (dd, J=8.9, 3.7 Hz, 1H).

### Example 75

### Synthesis of 3 3-(5-(5-fluoroindoline-1-carbonyl)-1-oxoisoindolin-2-yl)piperidine-2,6-dione

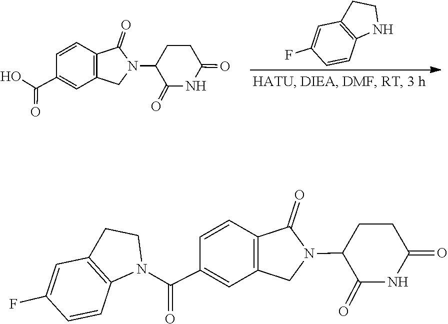 [SMILES: O=C1CCC(N2Cc3cc(C(=O)O)ccc3C2=O)C(=O)N1 ; Fc1ccc2c(c1)CCN2 ; O=C1CCC(N2Cc3cc(C(=O)N4CCc5cc(F)ccc54)ccc3C2=O)C(=O)N1]

Step 1. Preparation of 3-(5-(5-fluoroindoline-1-carbonyl)-1-oxoisoindolin-2-yl)piperidine-2,6-dione. A solution of 2-(2,6-dioxopiperidin-3-yl)-1-oxoisoindole-5-carboxylic acid (200 mg, 0.694 mmol, 1 equiv) and DIEA (269.0 mg, 2.082 mmol, 3 equiv) in DMF (2.5 mL) was stirred for 5 min at room temperature under air atmosphere. To the above mixture was added 5-fluoro-2,3-dihydro-1H-indole (114.2 mg, 0.833 mmol, 1.2 equiv) and HATU (395.7 mg, 1.041 mmol, 1.5 equiv) in portions 5 min at room temperature. The resulting mixture was stirred for additional 3 h at room temperature. The resulting mixture was diluted with DMF (2 mL). The crude product (50 mg) was purified by Prep-HPLC to afford 3-(5-(5-fluoroindoline-1-carbonyl)-1-oxoisoindolin-2-yl)piperidine-2,6-dione (52 mg, 18%) as an off-white solid.

LC-MS (ES, m/z)=408 [M+1]⁺. ¹H NMR (400 MHz, DMSO-d₆) δ 11.03 (s, 1H), 8.11 (s, 1H), 7.87-7.80 (m, 2H), 7.72 (d, J=7.9 Hz, 1H), 7.17 (dd, J=8.6, 2.7 Hz, 1H), 7.07 (d, J=13.7 Hz, 1H), 5.15 (dd, J=13.3, 5.1 Hz, 1H), 4.54 (d, J=17.5 Hz, 1H), 4.40 (d, J=17.6 Hz, 1H), 4.02 (t, J=8.0 Hz, 2H), 3.10 (t, J=8.3 Hz, 2H), 2.93 (ddd, J=18.2, 13.7, 5.4 Hz, 1H), 2.62 (dt, J=15.5, 3.0 Hz, 1H), 2.41 (td, J=13.2, 4.5 Hz, 1H), 2.04 (dtt, J=12.7, 5.4, 2.3 Hz, 1H).

### Example 76

### Synthesis of 3-(5-(7-fluoroindoline-1-carbonyl)-1-oxoisoindolin-2-yl)piperidine-2,6-dione

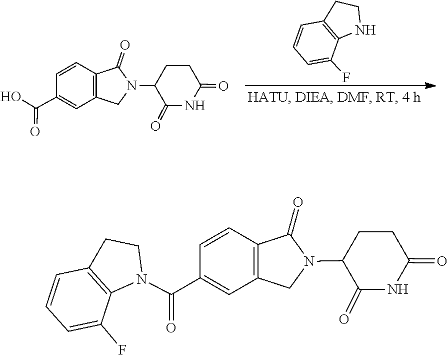 [SMILES: O=C1CCC(N2Cc3cc(C(=O)O)ccc3C2=O)C(=O)N1 ; Fc1cccc2c1NCC2 ; O=C1CCC(N2Cc3cc(C(=O)N4CCc5cccc(F)c54)ccc3C2=O)C(=O)N1]

Step 1. Preparation of 3-(5-(7-fluoroindoline-1-carbonyl)-1-oxoisoindolin-2-yl)piperidine-2,6-dione. To a stirred mixture of 2-(2,6-dioxopiperidin-3-yl)-1-oxoisoindole-5-carboxylic acid (200 mg, 0.694 mmol, 1 equiv) and 7-fluoro-2,3-dihydro-1H-indole (95.2 mg, 0.694 mmol, 1 equiv) in DMF (3 mL) were added DIEA (269.0 mg, 2.082 mmol, 3 equiv) and HATU (395.7 mg, 1.041 mmol, 1.5 equiv) at room temperature. The resulting mixture was stirred for 4 h at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-(7-fluoroindoline-1-carbonyl)-1-oxoisoindolin-2-yl)piperidine-2,6-dione (36 mg, 13%) as a white solid.

LC-MS (ES, m/z)=408.05 [M+1]⁺. ¹H NMR (400 MHz, DMSO-d₆) δ 11.02 (s, 1H), 7.93 (s, 1H), 7.82 (t, J=6.5 Hz, 2H), 7.20-7.09 (m, 2H), 7.09-7.00 (m, 1H), 5.15 (dd, J=13.3, 5.1 Hz, 1H), 4.53 (d, J=17.6 Hz, 1H), 4.41 (d, J=17.6 Hz, 1H), 4.12 (t, J=7.8 Hz, 2H), 3.12 (t, J=7.8 Hz, 2H), 2.93 (ddd, J=16.9, 13.7, 5.4 Hz, 1H), 2.61 (d, J=17.2 Hz, 1H), 2.41 (td, J=13.1, 4.5 Hz, 1H), 2.08-2.00 (m, 1H), 1.24 (s, 1H).

### Example 77

### Synthesis of 3-(1-oxo-5-(4-(trifluoromethyl)indoline-1-carbonyl)isoindolin-2-yl)piperidine-2,6-dione

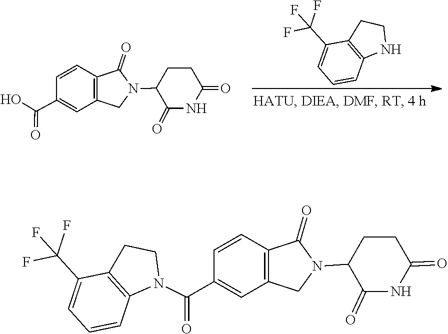 [SMILES: O=C1CCC(N2Cc3cc(C(=O)O)ccc3C2=O)C(=O)N1 ; FC(F)(F)c1cccc2c1CCN2 ; O=C1CCC(N2Cc3cc(C(=O)N4CCc5c4cccc5C(F)(F)F)ccc3C2=O)C(=O)N1]

Step 1. Preparation of 3-(1-oxo-5-(4-(trifluoromethyl)indoline-1-carbonyl)isoindolin-2-yl)piperidine-2,6-dione. A solution of 2-(2,6-dioxopiperidin-3-yl)-1-oxo-3H-isoindole-5-carboxylic acid (200 mg, 0.694 mmol, 1 equiv) and DIEA (269.0 mg, 2.082 mmol, 3 equiv) in DMF (3 mL) was stirred for 5 min at room temperature under air atmosphere. To the above mixture was added 4-(trifluoromethyl)-2,3-dihydro-1H-indole (155.8 mg, 0.833 mmol, 1.2 equiv) and HATU (395.7 mg, 1.041 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The crude product was purified by Prep-HPLC to afford 3-(1-oxo-5-(4-(trifluoromethyl)indoline-1-carbonyl)isoindolin-2-yl)piperidine-2,6-dione (67 mg, 21%) as an off-white solid.

LC-MS (ES, m/z)=458 [M+1]⁺. ¹H NMR (400 MHz, DMSO-d₆) δ 11.03 (s, 1H), 8.33 (s, 1H), 7.89-7.82 (m, 2H), 7.74 (d, J=7.9 Hz, 1H), 7.42 (t, J=13.0 Hz, 2H), 5.16 (dd, J=13.3, 5.1 Hz, 1H), 4.64-4.33 (m, 2H), 4.09 (t, J=8.4 Hz, 2H), 3.25 (t, J=8.3 Hz, 2H), 2.93 (ddd, J=18.4, 13.6, 5.3 Hz, 1H), 2.62 (d, J=16.9 Hz, 1H), 2.45 (d, J=4.5 Hz, 1H), 2.11-1.96 (m, 1H).

### Example 78

### Synthesis of 3-(1-oxo-5-(5-(trifluoromethyl)indoline-1-carbonyl)isoindolin-2-yl)piperidine-2,6-dione

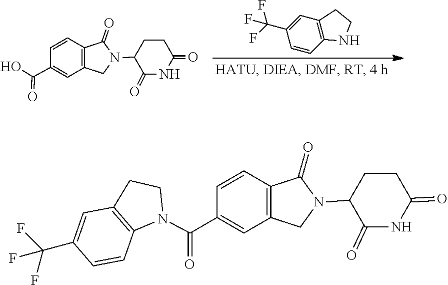 [SMILES: O=C1CCC(N2Cc3cc(C(=O)O)ccc3C2=O)C(=O)N1 ; FC(F)(F)c1ccc2c(c1)CCN2 ; O=C1CCC(N2Cc3cc(C(=O)N4CCc5cc(C(F)(F)F)ccc54)ccc3C2=O)C(=O)N1]

Step 1. Preparation of 3-(1-oxo-5-(5-(trifluoromethyl)indoline-1-carbonyl)isoindolin-2-yl)piperidine-2,6-dione. To a stirred solution of 2-(2,6-dioxopiperidin-3-yl)-1-oxo-3H-isoindole-5-carboxylic acid (200 mg, 0.694 mmol, 1 equiv), 5-(trifluoromethyl)-2,3-dihydro-1H-indole (129.9 mg, 0.694 mmol, 1 equiv) and DIEA (269.0 mg, 2.082 mmol, 3 equiv) in DMF (3 ml) were added HATU (395.7 mg, 1.041 mmol, 1.5 equiv) in portions at room temperature. The resulting mixture was stirred for 3 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 30 min; detector, UV 254 nm. This resulted in 3-{1-oxo-5-[5-(trifluoromethyl)-2,3-dihydroindole-1-carbonyl]-3H-isoindol-2-yl}piperidine-2,6-dione (88.4 mg, 27.85%) as an off-white solid.

LC-MS (ES, m/z)=458 [M+1]⁺. ¹H NMR (400 MHz, DMSO-d₆) δ 11.03 (s, 1H), 8.18 (s, 1H), 7.89-7.82 (m, 2H), 7.74 (d, J=7.8 Hz, 1H), 7.66 (s, 1H), 7.59 (s, 1H), 5.16 (dd, J=13.3, 5.1 Hz, 1H), 4.59-4.37 (m, 2H), 4.07 (t, J=8.5 Hz, 2H), 3.17 (t, J=8.4 Hz, 2H), 2.93 (ddd, J=17.2, 13.5, 5.4 Hz, 1H), 2.61 (d, J=17.5 Hz, 1H), 2.41 (td, J=13.2, 4.6 Hz, 1H), 2.08-2.00 (m, 1H).

### Example 79

### Synthesis of 3-(1-oxo-5-(6-(trifluoromethyl)indoline-1-carbonyl)isoindolin-2-yl)piperidine-2,6-dione

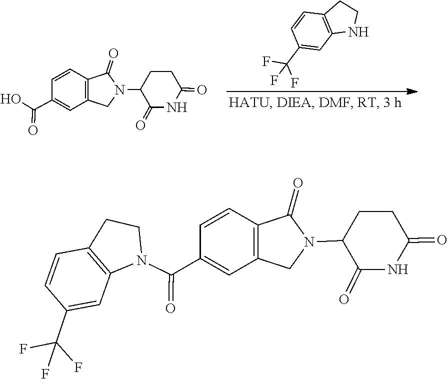 [SMILES: O=C1CCC(N2Cc3cc(C(=O)O)ccc3C2=O)C(=O)N1 ; FC(F)(F)c1ccc2c(c1)NCC2 ; O=C1CCC(N2Cc3cc(C(=O)N4CCc5ccc(C(F)(F)F)cc54)ccc3C2=O)C(=O)N1]

Step 1. Preparation of 3-(1-oxo-5-(6-(trifluoromethyl)indoline-1-carbonyl)isoindolin-2-yl)piperidine-2,6-dione. A solution of 2-(2,6-dioxopiperidin-3-yl)-1-oxoisoindole-5-carboxylic acid (200 mg, 0.694 mmol, 1 equiv) and DIEA (269.0 mg, 2.082 mmol, 3 equiv) in DMF (2.5 mL) was stirred for 5 min at room temperature under air atmosphere. To the above mixture was added 6-(trifluoromethyl)-2,3-dihydro-1H-indole (155.8 mg, 0.833 mmol, 1.2 equiv) and HATU (395.7 mg, 1.041 mmol, 1.5 equiv) in portions 5 min at room temperature. The resulting mixture was stirred for additional 3 h at room temperature. The resulting mixture was diluted with DMF (2 mL). The crude product was purified by Prep-HPLC to afford 3-(1-oxo-5-(6-(trifluoromethyl)indoline-1-carbonyl)isoindolin-2-yl)piperidine-2,6-dione (37 mg, 11%) as an off-white solid.

LC-MS (ES, m/z)=458 [M+1]⁺. ¹H NMR (400 MHz, DMSO-d₆) δ 11.03 (s, 1H), 8.40 (s, 1H), 7.85 (d, J=8.0 Hz, 2H), 7.78-7.71 (m, 1H), 7.52 (d, J=7.8 Hz, 1H), 7.45 (d, J=8.1 Hz, 1H), 5.16 (dd, J=13.3, 5.1 Hz, 1H), 4.55 (d, J=17.6 Hz, 1H), 4.41 (d, J=17.7 Hz, 1H), 4.07 (t, J=8.3 Hz, 2H), 3.19 (t, J=8.3 Hz, 2H), 2.98-2.88 (m, 1H), 2.61 (d, J=17.0 Hz, 1H), 2.40 (dd, J=13.4, 4.4 Hz, 1H), 2.07-2.00 (m, 1H).

### Example 80

### Synthesis of 3-(1-oxo-5-(5-(trifluoromethoxy)indoline-1-carbonyl)isoindolin-2-yl)piperidine-2,6-dione

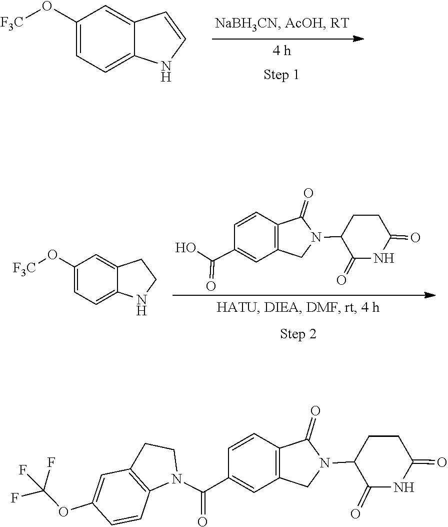 [SMILES: FC(F)(F)Oc1ccc2[nH]ccc2c1 ; FC(F)(F)Oc1ccc2c(c1)CCN2 ; O=C1CCC(N2Cc3cc(C(=O)O)ccc3C2=O)C(=O)N1 ; O=C1CCC(N2Cc3cc(C(=O)N4CCc5cc(OC(F)(F)F)ccc54)ccc3C2=O)C(=O)N1]

Step 1. Preparation of 5-(trifluoromethoxy)indoline. To a stirred mixture of 5-(trifluoromethoxy)-1H-indole (500 mg, 2.486 mmol, 1 equiv) in AcOH (10.00 mL) was added NaBH₃CN (624.8 mg, 9.944 mmol, 4 equiv) in portions at 0° C. The resulting mixture was stirred for 4 h at room temperature. The reaction was quenched by the addition of sat. NH₄Cl (aq.) (10 mL) at 0° C. The resulting mixture was extracted with EtOAc (3×5 mL). The combined organic layers were washed with brine (2×4 mL), dried over anhydrous Na₂SO₄. After filtration, the filtrate was concentrated under reduced pressure to afford 5-(trifluoromethoxy)indoline (300 mg, 59%) as a yellow oil.

Step 2. Preparation of 3-(1-oxo-5-(5-(trifluoromethoxy)indoline-1-carbonyl)isoindolin-2-yl)piperidine-2,6-dione. To a stirred mixture of 5-(trifluoromethoxy)indoline (150 mg, 0.738 mmol, 1 equiv) and 2-(2,6-dioxopiperidin-3-yl)-1-oxoisoindole-5-carboxylic acid (212.8 mg, 0.738 mmol, 1 equiv) in DMF (3 mL) were added DIEA (286.3 mg, 2.214 mmol, 3 equiv) and HATU (421.1 mg, 1.107 mmol, 1.5 equiv) at room temperature. The resulting mixture was stirred for 4 h at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(1-oxo-5-(5-(trifluoromethoxy)indoline-1-carbonyl)isoindolin-2-yl)piperidine-2,6-dione (80 mg, 22%) as a white solid.

LC-MS (ES, m/z)=473.95 [M+1]⁺. ¹H NMR (300 MHz, DMSO-d₆) δ 11.04 (s, 1H), 7.89-7.81 (m, 2H), 7.73 (d, J=7.8 Hz, 1H), 7.34 (s, 1H), 7.23 (s, 1H), 5.17 (dd, J=13.2, 5.0 Hz, 1H), 4.55 (d, J=17.7 Hz, 1H), 4.41 (d, J=17.6 Hz, 1H), 4.05 (t, J=8.5 Hz, 2H), 3.14 (t, J=8.3 Hz, 2H), 2.93 (td, J=12.8, 6.5 Hz, 1H), 2.62 (d, J=16.6 Hz, 1H), 2.47-2.34 (m, 1H), 2.09-1.99 (m, 1H), 1.24 (s, 1H).

### Example 81

### Synthesis of 3-(1-oxo-5-(6-(trifluoromethoxy)indoline-1-carbonyl)isoindolin-2-yl)piperidine-2,6-dione

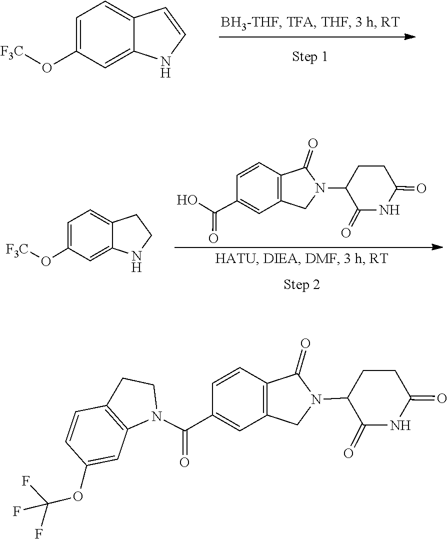 [SMILES: FC(F)(F)Oc1ccc2cc[nH]c2c1 ; FC(F)(F)Oc1ccc2c(c1)NCC2 ; O=C1CCC(N2Cc3cc(C(=O)O)ccc3C2=O)C(=O)N1 ; O=C1CCC(N2Cc3cc(C(=O)N4CCc5ccc(OC(F)(F)F)cc54)ccc3C2=O)C(=O)N1]

Step 1. Preparation of 6-(trifluoromethoxy)indoline. A solution of 6-(trifluoromethoxy)-1H-indole (200 mg, 0.994 mmol, 1 equiv) in THF (5 mL) was treated with BH₃-THF (2.9 mL) for 30 min at 0° C. under nitrogen atmosphere followed by the addition of TFA (2.9 mL) dropwise at 0° C. The resulting mixture was stirred for additional 3 h at room temperature. The reaction was quenched with Water at 0° C. The residue was purified by reverse-phase flash chromatography to afford 6-(trifluoromethoxy)indoline (110 mg, 54%) as a light yellow oil.

Step 2. Preparation of 3-(1-oxo-5-(6-(trifluoromethoxy)indoline-1-carbonyl)isoindolin-2-yl)piperidine-2,6-dione. A solution of 2-(2,6-dioxopiperidin-3-yl)-1-oxoisoindole-5-carboxylic acid (80 mg, 0.278 mmol, 1 equiv) and DIEA (107.6 mg, 0.834 mmol, 3 equiv) in DMF (3 mL) was stirred for 5 min at room temperature under air atmosphere. To the above mixture was added 6-(trifluoromethoxy)indoline (67.7 mg, 0.334 mmol, 1.2 equiv) and HATU (158.3 mg, 0.417 mmol, 1.5 equiv) in portions over 5 min at room temperature. The resulting mixture was stirred for additional 3 h at room temperature. The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (1:1) to afford 3-(1-oxo-5-(6-(trifluoromethoxy)indoline-1-carbonyl)isoindolin-2-yl)piperidine-2,6-dione (5 mg, 4%) as an off-white solid.

LC-MS (ES, m/z)=474 [M+1]⁺. ¹H NMR (400 MHz, DMSO-d₆) δ 11.02 (s, 1H), 8.05 (s, 1H), 7.88-7.81 (m, 2H), 7.74 (d, J=7.8 Hz, 1H), 7.40 (d, J=8.2 Hz, 1H), 7.07 (d, J=8.3 Hz, 1H), 5.16 (dd, J=13.3, 5.1 Hz, 1H), 4.54 (d, J=17.6 Hz, 1H), 4.41 (d, J=17.6 Hz, 1H), 4.06 (t, J=8.3 Hz, 2H), 3.14 (s, 1H), 3.09 (s, 2H), 2.93 (ddd, J=17.9, 13.6, 5.4 Hz, 1H), 2.61 (d, J=17.3 Hz, 1H), 2.46-2.35 (m, 1H), 2.12-1.91 (m, 1H), 1.24 (s, 1H).

### Example 82

### Synthesis of 3-(5-(4-methoxyindoline-1-carbonyl)-1-oxoisoindolin-2-yl)piperidine-2,6-dione

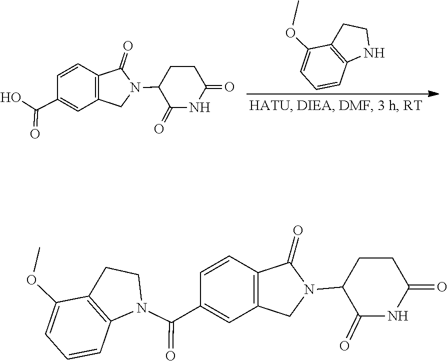 [SMILES: O=C1CCC(N2Cc3cc(C(=O)O)ccc3C2=O)C(=O)N1 ; COc1cccc2c1CCN2 ; COc1cccc2c1CCN2C(=O)c1ccc2c(c1)CN(C1CCC(=O)NC1=O)C2=O]

Step 1. Preparation of 3-(5-(4-methoxyindoline-1-carbonyl)-1-oxoisoindolin-2-yl)piperidine-2,6-dione. A solution of 2-(2,6-dioxopiperidin-3-yl)-1-oxoisoindole-5-carboxylic acid (200 mg, 0.694 mmol, 1 equiv) and DIEA (269.0 mg, 2.082 mmol, 3 equiv) in DMF (3 mL) was stirred for 5 min at room temperature under air atmosphere. To the above mixture was added 4-methoxy-2,3-dihydro-1H-indole (124.2 mg, 0.833 mmol, 1.2 equiv) and HATU (395.7 mg, 1.041 mmol, 1.5 equiv) in portions over 5 min at room temperature. The resulting mixture was stirred for additional 3 h at room temperature. The reaction was quenched with Water at 0° C. The resulting mixture was concentrated under reduced pressure. The crude product was purified by Prep-HPLC to afford 3-(5-(4-methoxyindoline-1-carbonyl)-1-oxoisoindolin-2-yl)piperidine-2,6-dione (63 mg, 22%) as an off-white solid.

LC-MS (ES, m/z)=420 [M+1]⁺. ¹H NMR (400 MHz, DMSO-d₆) δ 11.03 (s, 1H), 7.86-7.63 (m, 4H), 7.20 (s, 1H), 6.74 (s, 1H), 5.16 (dd, J=13.3, 5.1 Hz, 1H), 4.60-4.29 (m, 2H), 4.01 (s, 2H), 3.80 (s, 3H), 3.01-2.86 (m, 3H), 2.61 (d, J=17.0 Hz, 1H), 2.42 (qd, J=13.1, 4.3 Hz, 1H), 2.10-1.99 (m, 1H).

### Example 83

### Synthesis of 3-(5-(5-methoxyindoline-1-carbonyl)-1-oxoisoindolin-2-yl)piperidine-2,6-dione

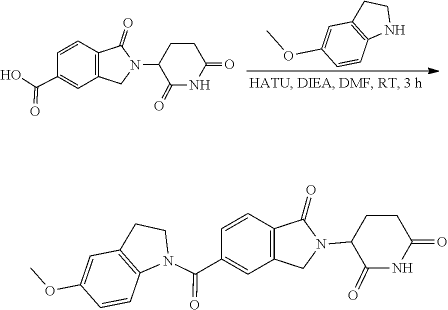 [SMILES: O=C1CCC(N2Cc3cc(C(=O)O)ccc3C2=O)C(=O)N1 ; COc1ccc2c(c1)CCN2 ; COc1ccc2c(c1)CCN2C(=O)c1ccc2c(c1)CN(C1CCC(=O)NC1=O)C2=O]

Step 1. Preparation of 3-(5-(5-methoxyindoline-1-carbonyl)-1-oxoisoindolin-2-yl)piperidine-2,6-dione. A solution of 2-(2,6-dioxopiperidin-3-yl)-1-oxoisoindole-5-carboxylic acid (200 mg, 0.694 mmol, 1 equiv) and DIEA (269.0 mg, 2.082 mmol, 3 equiv) in DMF (2.5 mL) was stirred for 5 min at room temperature under air atmosphere. To the above mixture was added 5-fluoro-2,3-dihydro-1H-indole (114.2 mg, 0.833 mmol, 1.2 equiv) and HATU (395.7 mg, 1.041 mmol, 1.5 equiv) in portions 5 min at room temperature. The resulting mixture was stirred for additional 3 h at room temperature. The resulting mixture was filtered. the filter cake was washed with acetonitrile (3×7 mL). The filtrate was concentrated under reduced pressure. The resulting mixture was concentrated under reduced pressure. The filtrate (5 mL) was purified by Prep-HPLC to afford 3-(5-(5-methoxyindoline-1-carbonyl)-1-oxoisoindolin-2-yl)piperidine-2,6-dione (68 mg, 23%) as an off-white solid.

LC-MS (ES, m/z)=420 [M+1]⁺. ¹H NMR (300 MHz, DMSO-d₆) δ 11.04 (s, 1H), 8.04 (s, 1H), 7.83 (d, J=7.5 Hz, 2H), 7.72 (s, 1H), 6.91 (d, J=2.6 Hz, 1H), 6.81 (s, 1H), 5.16 (dd, J=13.3, 5.0 Hz, 1H), 4.54 (d, J=17.6 Hz, 1H), 4.40 (d, J=17.6 Hz, 1H), 3.99 (s, 2H), 3.74 (s, 3H), 3.07 (t, J=8.1 Hz, 2H), 3.02-2.85 (m, 1H), 2.62 (d, J=17.0 Hz, 1H), 2.42 (dt, J=13.3, 6.9 Hz, 2H), 2.04 (d, J=11.9 Hz, 1H).

### Example 84

### Synthesis of 3-(5-(6-methoxyindoline-1-carbonyl)-1-oxoisoindolin-2-yl)piperidine-2,6-dione

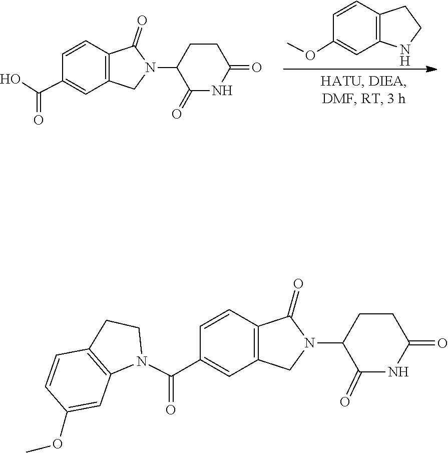

Step 1. Preparation of 3-(5-(6-methoxyindoline-1-carbonyl)-1-oxoisoindolin-2-yl)piperidine-2,6-dione. To a stirred solution of 2-(2,6-dioxopiperidin-3-yl)-1-oxoisoindole-5-carboxylic acid (200 mg, 0.694 mmol, 1 equiv), 6-methoxy-2,3-dihydro-1H-indole (103.5 mg, 0.694 mmol, 1 equiv) and DIEA (269.0 mg, 2.082 mmol, 3 equiv) in DMF (3 ml) were added HATU (395.7 mg, 1.041 mmol, 1.5 equiv) in portions at room temperature. The resulting mixture was stirred for 3 h at room temperature under nitrogen atmosphere. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The crude product was purified by Prep-HPLC to afford 3-(5-(6-methoxyindoline-1-carbonyl)-1-oxoisoindolin-2-yl)piperidine-2,6-dione (61 mg, 21%) as an off-white solid.

LC-MS (ES, m/z)=420.05 [M+1]⁺. ¹H NMR (300 MHz, DMSO-d₆) δ 11.02 (s, 1H), 7.84 (d, J=7.7 Hz, 3H), 7.70 (d, J=8.0 Hz, 1H), 7.17 (d, J=8.2 Hz, 1H), 6.66 (s, 1H), 5.16 (dd, J=13.3, 5.1 Hz, 1H), 4.57-4.35 (m, 2H), 4.00 (s, 2H), 3.72 (s, 2H), 3.00 (t, J=8.4 Hz, 3H), 2.61 (d, J 17.4 Hz, 2H), 2.43 (d, J=13.6 Hz, 1H), 2.05 (s, 1H).

### Example 85

### Synthesis of 3-(5-(7-methoxyindoline-1-carbonyl)-1-oxoisoindolin-2-yl)piperidine-2,6-dione

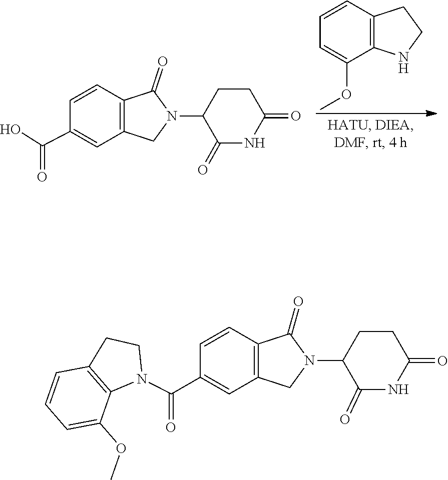

Step 1. Preparation of 3-(5-(7-methoxyindoline-1-carbonyl)-1-oxoisoindolin-2-yl)piperidine-2,6-dione. To a stirred mixture of 2-(2,6-dioxopiperidin-3-yl)-1-oxoisoindole-5-carboxylic acid (200 mg, 0.694 mmol, 1 equiv) and 7-methoxy-2,3-dihydro-1H-indole (103.5 mg, 0.694 mmol, 1 equiv) in DMF (3 mL) were added DIEA (269.0 mg, 2.082 mmol, 3 equiv) and HATU (395.7 mg, 1.041 mmol, 1.5 equiv) at room temperature. The resulting mixture was stirred for 4 h at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse-phase flash chromatography to afford 3-(5-(7-methoxyindoline-1-carbonyl)-1-oxoisoindolin-2-yl)piperidine-2,6-dione (54 mg, 18%) as a white solid.

LC-MS (ES, m/z)=420.10 [M+1]⁺. ¹H NMR (400 MHz, DMSO-d₆) δ 11.00 (s, 1H), 7.76 (d, J=8.3 Hz, 2H), 7.64 (d, J=7.6 Hz, 1H), 7.10-7.02 (m, 1H), 6.93 (d, J=7.4 Hz, 1H), 6.80 (d, J 8.1 Hz, 1H), 5.14 (dd, J=13.3, 5.1 Hz, 1H), 4.50 (d, J=17.6 Hz, 1H), 4.36 (d, J=17.5 Hz, 1H), 4.09 (t, J=7.6 Hz, 2H), 3.06 (t, J=7.6 Hz, 2H), 2.92 (ddd, J=17.5, 13.6, 5.3 Hz, 1H), 2.61 (d, J=17.0 Hz, 1H), 2.45-2.34 (m, 1H), 2.08-1.99 (m, 1H), 1.24 (s, 1H).

### Example 86

### Synthesis of 3-(5-(4-chloro-7-fluoroindoline-1-carbonyl)-1-oxoisoindolin-2-yl)piperidine-2,6-dione

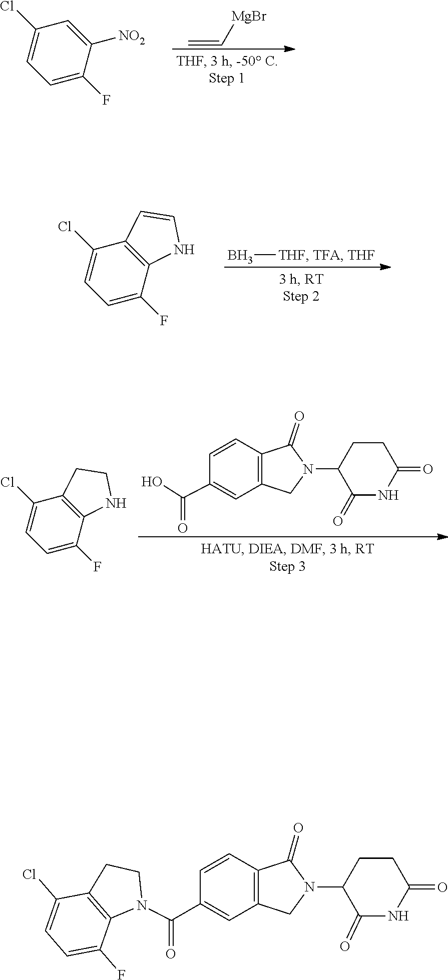

Step 1. Preparation of 4-chloro-7-fluoro-1H-indole. A solution of 4-chloro-1-fluoro-2-nitrobenzene (2 g, 11.393 mmol, 1 equiv) in THF (20 mL) was added bromo(ethenyl)magnesium (18 mL) dropwise at −50° C. The resulting mixture was stirred for additional 3 h at room temperature. The reaction was quenched with sat. NH₄Cl (aq.) at 0° C. The resulting mixture was extracted with EtOAc (3×50 mL). The combined organic layers were washed with water (3×30 mL), dried over anhydrous Na₂SO₄. After filtration, the filtrate was concentrated under reduced pressure. The residue was purified by reverse-phase flash chromatography to afford 4-chloro-7-fluoro-1H-indole (400 mg, 21%) as a light-yellow oil.

Step 2. Preparation of 4-chloro-7-fluoroindoline. A solution of 4-chloro-7-fluoro-1H-indole (100 mg, 0.590 mmol, 1 equiv) in THF (2 mL) was treated with TFA (0.6 mL) for 5 min at room temperature under nitrogen atmosphere followed by the addition of BH₃-THF (0.9 mL) dropwise at 0° C. The resulting mixture was stirred for additional 3 h at room temperature. The reaction was quenched with sat. NH₄Cl (aq.) at 0° C. The resulting mixture was extracted with EtOAc (3×40 mL). The combined organic layers were washed with brine (3×40 mL), dried over anhydrous Na₂SO₄. After filtration, the filtrate was concentrated under reduced pressure. The residue was purified by reverse-phase flash chromatography to afford 4-chloro-7-fluoroindoline (40 mg, 40%) as a light-yellow solid.

Step 3. Preparation of 3-(5-(4-chloro-7-fluoroindoline-1-carbonyl)-1-oxoisoindolin-2-yl)piperidine-2,6-dione. A solution of 4-chloro-7-fluoroindoline (17.9 mg, 0.104 mmol, 1 equiv), DIEA (40.4 mg, 0.312 mmol, 3 equiv) and 2-(2,6-dioxopiperidin-3-yl)-1-oxoisoindole-5-carboxylic acid (30 mg, 0.104 mmol, 1 equiv) in DMF (1 mL) was stirred for 10 min at room temperature. To the above mixture was added HATU (59.4 mg, 0.156 mmol, 1.5 equiv) at room temperature. The resulting mixture was stirred for additional 3 h at room temperature. The resulting mixture was diluted with water and extracted with CH₂Cl₂ (3×20 mL). The combined organic layers were washed with brine (3×30 mL), dried over anhydrous Na₂SO₄. After filtration, the filtrate was concentrated under reduced pressure. The crude product was purified by Prep-HPLC to afford 3-(5-(4-chloro-7-fluoroindoline-1-carbonyl)-1-oxoisoindolin-2-yl)piperidine-2,6-dione (3 mg, 7%) as an off-white solid.

LC-MS (ES, m/z)=440.05 [M−1]⁻. ¹H NMR (400 MHz, DMSO-d₆) δ 11.01 (s, 1H), 7.94 (s, 1H), 7.83 (t, J=5.9 Hz, 2H), 7.24-7.10 (m, 2H), 5.15 (dd, J=13.3, 5.1 Hz, 1H), 4.54 (d, J=17.7 Hz, 1H), 4.41 (d, J=17.6 Hz, 1H), 4.17 (t, J=7.8 Hz, 2H), 3.14 (t, J=7.8 Hz, 2H), 2.96-2.86 (m, 1H), 2.62 (d, J=16.7 Hz, 1H), 2.41 (dt, J=14.0, 7.0 Hz, 1H), 2.08-2.01 (m, 1H), 1.24 (s, 1H).

### Example 87

### Synthesis of 3-(5-(4-fluoroisoindoline-2-carbonyl)-1-oxoisoindolin-2-yl)piperidine-2,6-dione

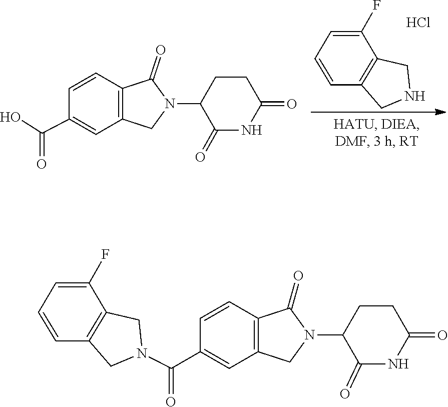

Step 1. Preparation of 3-(5-(4-fluoroisoindoline-2-carbonyl)-1-oxoisoindolin-2-yl)piperidine-2,6-dione. A solution of 4-fluoro-2,3-dihydro-1H-isoindole hydrochloride (48.2 mg, 0.278 mmol, 1 equiv), DIEA (107.6 mg, 0.834 mmol, 3 equiv) and 2-(2,6-dioxopiperidin-3-yl)-1-oxoisoindole-5-carboxylic acid (80 mg, 0.278 mmol, 1.00 equiv) in DMF (2 mL) was stirred for 10 min at room temperature. To the above mixture was added HATU (158.3 mg, 0.417 mmol, 1.5 equiv) in portions at room temperature. The resulting mixture was stirred for additional 3 h at room temperature. The resulting mixture was diluted with water. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reverse-phase flash chromatography to afford 3-(5-(4-fluoroisoindoline-2-carbonyl)-1-oxoisoindolin-2-yl)piperidine-2,6-dione (60 mg, 53%) as an off-white solid.

LC-MS (ES, m/z)=408.15 [M+1]⁺. ¹H NMR (400 MHz, DMSO-d₆) δ 11.01 (s, 1H), 7.87-7.81 (m, 2H), 7.73 (dd, J=6.4, 2.6 Hz, 1H), 7.43-7.28 (m, 1H), 7.17-7.08 (m, 2H), 5.16 (dd, J=13.3, 5.1 Hz, 1H), 4.94 (s, 2H), 4.82 (d, J=10.0 Hz, 2H), 4.58-4.37 (m, 2H), 2.93 (ddd, J=18.2, 13.6, 5.4 Hz, 1H), 2.62 (d, J=17.3 Hz, 1H), 2.43 (dd, J=13.2, 4.5 Hz, 1H), 2.16-1.97 (m, 1H).

### Example 88

### Synthesis of 3-(5-(5-fluoroisoindoline-2-carbonyl)-1-oxoisoindolin-2-yl)piperidine-2,6-dione

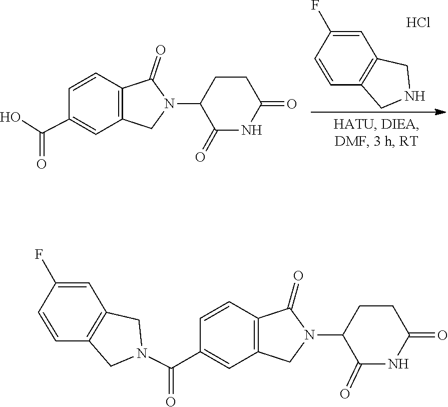

Step 1. Preparation of 3-(5-(5-fluoroisoindoline-2-carbonyl)-1-oxoisoindolin-2-yl)piperidine-2,6-dione. A solution of 5-fluoro-2,3-dihydro-1H-isoindole hydrochloride (48.2 mg, 0.278 mmol, 1 equiv), DIEA (107.6 mg, 0.834 mmol, 3 equiv) and 2-(2,6-dioxopiperidin-3-yl)-1-oxo-3H-isoindole-5-carboxylic acid (80 mg, 0.278 mmol, 1.00 equiv) in DMF (2 mL) was stirred for 10 min at room temperature. To the above mixture was added HATU (158.3 mg, 0.417 mmol, 1.5 equiv) in portions at room temperature. The resulting mixture was stirred for additional 3 h at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reverse-phase flash chromatography to afford 3-(5-(5-fluoroisoindoline-2-carbonyl)-1-oxoisoindolin-2-yl)piperidine-2,6-dione (75 mg, 66%) as an off-white solid.

LC-MS (ES, m/z)=408.15 [M+1]⁺. ¹HNMR (400 MHz, DMSO-d₆) δ 11.01 (s, 1H), 7.87-7.79 (m, 2H), 7.71 (s, 1H), 7.51-7.00 (m, 3H), 5.16 (dd, J=13.3, 5.1 Hz, 1H), 4.87 (d, J=13.8 Hz, 2H), 4.73 (d, J=12.4 Hz, 2H), 4.54-4.33 (m, 2H), 2.93 (ddd, J=17.8, 13.6, 5.4 Hz, 1H), 2.62 (dt, J=15.6, 3.2 Hz, 1H), 2.42 (tt, J=13.2, 6.5 Hz, 1H), 2.10-1.99 (m, 1H).

### Example 89

### Synthesis of N-(benzo[d]isothiazol-3-yl)-2-(2,6-dioxopiperidin-3-yl)-1-oxoisoindoline-5-carboxamide

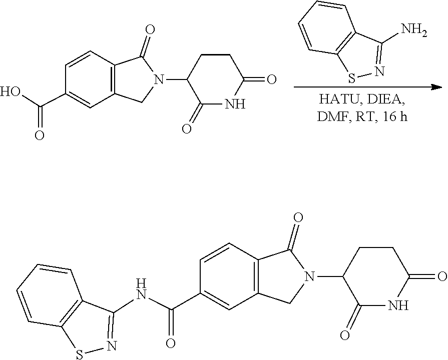 [SMILES: O=C1CCC(N2Cc3cc(C(=O)O)ccc3C2=O)C(=O)N1 ; Nc1nsc2ccccc12 ; O=C1CCC(N2Cc3cc(C(=O)Nc4nsc5ccccc45)ccc3C2=O)C(=O)N1]

Step 1. Preparation of N-(benzo[d]isothiazol-3-yl)-2-(2,6-dioxopiperidin-3-yl)-1-oxoisoindoline-5-carboxamide. To a stirred solution of 2-(2,6-dioxopiperidin-3-yl)-1-oxoisoindole-5-carboxylic acid (100 mg, 0.347 mmol, 1 equiv), 1,2-benzothiazol-3-amine (52.1 mg, 0.347 mmol, 1 equiv) and DIEA (134.5 mg, 1.041 mmol, 3 equiv) in DMF (2 mL) was added HATU (197.8 mg, 0.520 mmol, 1.5 equiv) in portions at room temperature under nitrogen atmosphere. The resulting mixture was stirred for overnight at room temperature under nitrogen atmosphere. The resulting mixture was diluted with water (25 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product (60 mg) was purified by Prep-HPLC to afford N-(benzo[d]isothiazol-3-yl)-2-(2,6-dioxopiperidin-3-yl)-1-oxoisoindoline-5-carboxamide (15 mg, 10%) as an off-white solid.

LC-MS (ES, m/z)=421.15 [M+1]⁺. ¹H NMR (400 MHz, DMSO-d₆) δ 11.38 (s, 1H), 11.02 (s, 1H), 8.28 (s, 1H), 8.25-8.15 (m, 2H), 8.03 (d, J=8.1 Hz, 1H), 7.91 (d, J=7.9 Hz, 1H), 7.65 (ddd, J=8.2, 6.9, 1.1 Hz, 1H), 7.51 (ddd, J=8.1, 6.9, 1.0 Hz, 1H), 5.18 (dd, J=13.3, 5.1 Hz, 1H), 4.58 (d, J=17.7 Hz, 1H), 4.46 (d, J=17.6 Hz, 1H), 2.94 (ddd, J=18.1, 13.6, 5.5 Hz, 1H), 2.62 (d, J=17.5 Hz, 1H), 2.42 (dd, J=13.3, 4.4 Hz, 1H), 2.08-2.03 (m, 1H), 1.38-1.21 (m, 2H), −0.06 (s, 1H).

### Example 90

### Synthesis of 2-(2,6-dioxopiperidin-3-yl)-1-oxo-N-(thiazol-4-yl)isoindoline-5-carboxamide

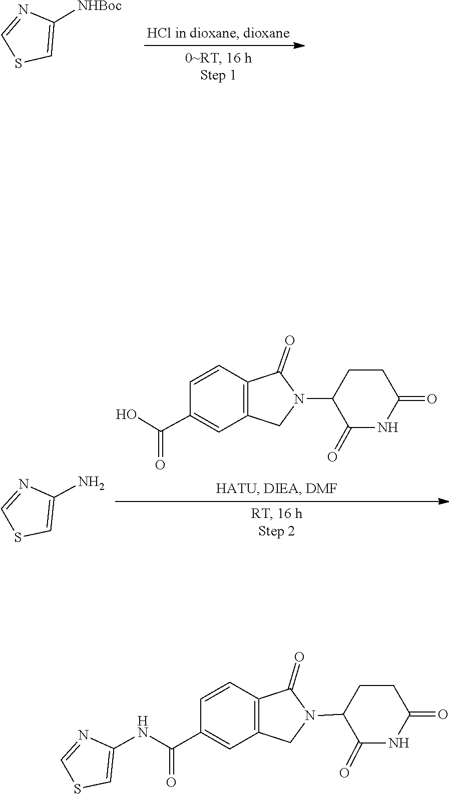 [SMILES: CC(C)(C)OC(=O)Nc1cscn1 ; O=C1CCC(N2Cc3cc(C(=O)O)ccc3C2=O)C(=O)N1 ; Nc1cscn1 ; O=C1CCC(N2Cc3cc(C(=O)Nc4cscn4)ccc3C2=O)C(=O)N1]

Step 1. Preparation of thiazol-4-amine. To a stirred solution of tert-butyl N-(1,3-thiazol-4-yl)carbamate (400 mg, 1.997 mmol, 1 equiv) in dioxane (4 mL) was added HCl (gas) in 1,4-dioxane (2 mL) dropwise at 0° C. under nitrogen atmosphere. The resulting mixture was stirred for overnight at room temperature under nitrogen atmosphere. The resulting mixture was concentrated under reduced pressure. This resulted in 1,3-thiazol-4-amine (150 mg, crude) as an off-white solid. The crude product was used in the next step directly without further purification.

Step 2. Preparation of 2-(2,6-dioxopiperidin-3-yl)-1-oxo-N-(thiazol-4-yl)isoindoline-5-carboxamide. To a stirred solution of 1,3-thiazol-4-amine (100 mg, 0.999 mmol, 1 equiv), 2-(2,6-dioxopiperidin-3-yl)-1-oxoisoindole-5-carboxylic acid (287.8 mg, 0.999 mmol, 1 equiv) and DIEA (387.2 mg, 2.997 mmol, 3 equiv) in DMF (2 mL) was added HATU (569.5 mg, 1.498 mmol, 1.5 equiv) in portions at room temperature under nitrogen atmosphere. The resulting mixture was stirred for overnight at room temperature under nitrogen atmosphere. The resulting mixture was diluted with water (50 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product (60 mg) was purified by Prep-HPLC to afford 2-(2,6-dioxopiperidin-3-yl)-1-oxo-N-(thiazol-4-yl)isoindoline-5-carboxamide (25 mg, 7%) as an off-white solid.

LC-MS (ES, m/z)=371.10 [M+1]⁺. ¹H NMR (400 MHz, DMSO-d₆) δ 11.63 (s, 1H), 11.01 (s, 1H), 9.06 (d, J=2.2 Hz, 1H), 8.24 (s, 1H), 8.14 (dd, J=7.9, 1.5 Hz, 1H), 7.91-7.81 (m, 2H), 5.16 (dd, J=13.3, 5.1 Hz, 1H), 4.59-4.38 (m, 2H), 2.93 (ddd, J=17.2, 13.6, 5.4 Hz, 1H), 2.62 (ddd, J=17.3, 4.5, 2.4 Hz, 1H), 2.43 (td, J=13.2, 4.5 Hz, 1H), 2.04 (dtd, J=12.9, 5.6, 5.0, 3.0 Hz, 1H), 1.25 (d, J=9.6 Hz, 1H).

### Example 91

### Synthesis of N-(benzo[b]thiophen-3-yl)-2-(2,6-dioxopiperidin-3-yl)-6-fluoro-1-oxoisoindoline-5-carboxamide

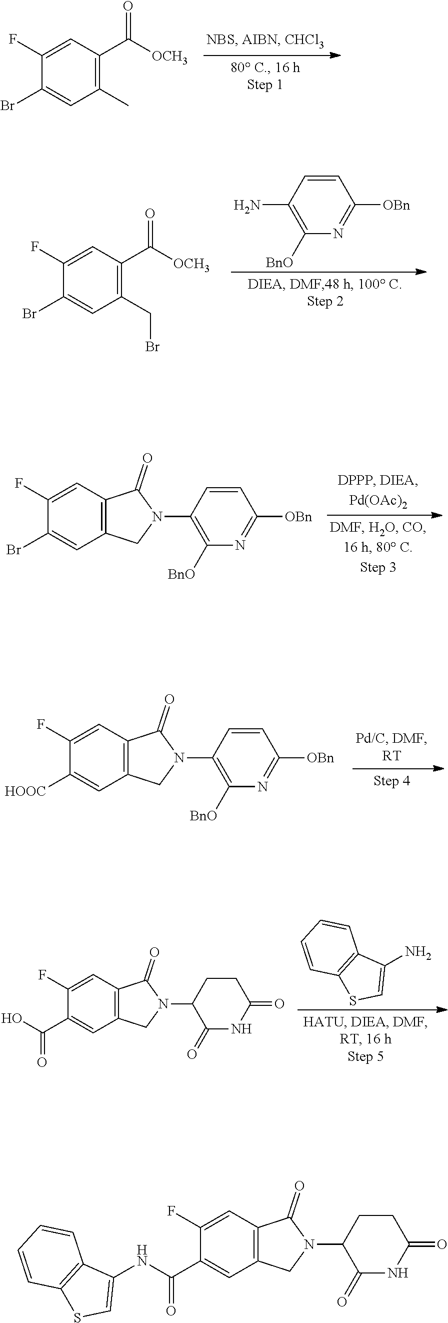

Step 1. Preparation of methyl 4-bromo-2-(bromomethyl)-5-fluorobenzoate. To a stirred solution of methyl 4-bromo-5-fluoro-2-methylbenzoate (1 g, 4.048 mmol, 1 equiv) and NBS (0.94 g, 5.262 mmol, 1.3 equiv) in chloroform (10 mL) was added AIBN (0.07 g, 0.405 mmol, 0.1 equiv) at room temperature under nitrogen atmosphere. The resulting mixture was stirred for overnight at 80° C. under nitrogen atmosphere. The mixture was allowed to cool down to room temperature. The resulting mixture was diluted with water (100 mL). The resulting mixture was extracted with CH₂Cl₂ (3×30 mL). The combined organic layers were washed with brine (2×30 mL), dried over anhydrous Na₂SO₄. After filtration, the filtrate was concentrated under reduced pressure to afford 4-bromo-2-(bromomethyl)-5-fluorobenzoate (1.5 g, crude) as a brown oil. The crude product was used in the next step directly without further purification.

Step 2. Preparation of 2-(2,6-bis(benzyloxy)pyridin-3-yl)-5-bromo-6-fluoroisoindolin-1-one. To a stirred solution of methyl 4-bromo-2-(bromomethyl)-5-fluorobenzoate (1.5 g, 4.602 mmol, 1 equiv) and 2,6-bis(benzyloxy)pyridin-3-amine (1.69 g, 5.522 mmol, 1.2 equiv) in DMF (10 mL) was added DIEA (1.78 g, 13.806 mmol, 3 equiv) at room temperature under nitrogen atmosphere. The resulting mixture was stirred for 48 h at 100° C. under nitrogen atmosphere. The mixture was allowed to cool down to room temperature. The resulting mixture was diluted with water (150 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reverse-phase flash chromatography to afford 2-(2,6-bis(benzyloxy)pyridin-3-yl)-5-bromo-6-fluoroisoindolin-1-one (1 g, 42%) as a brown oil.

Step 3. Preparation of 2-(2,6-bis(benzyloxy)pyridin-3-yl)-6-fluoro-1-oxoisoindoline-5-carboxylic acid. To a stirred solution of 2-(2,6-bis(benzyloxy)pyridin-3-yl)-5-bromo-6-fluoroisoindolin-1-one (1 g, 1.925 mmol, 1 equiv), DPPP (79.4 mg, 0.193 mmol, 0.1 equiv) in DMF (9 mL) and H₂O (1 mL) were added DIEA (746.5 mg, 5.775 mmol, 3 equiv) and Pd(OAc)₂ (86.4 mg, 0.385 mmol, 0.2 equiv) at room temperature under carbon monoxide atmosphere. The resulting mixture was stirred for overnight at 80° C. under carbon monoxide atmosphere. The mixture was allowed to cool down to room temperature. The resulting mixture was diluted with water (100 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by reverse-phase flash chromatography to afford 2-(2,6-bis(benzyloxy)pyridin-3-yl)-6-fluoro-1-oxoisoindoline-5-carboxylic acid (700 mg, 75%) as a brown solid.

Step 4. Preparation of 2-(2,6-dioxopiperidin-3-yl)-6-fluoro-1-oxoisoindoline-5-carboxylic acid. To a solution of 2-(2,6-bis(benzyloxy)pyridin-3-yl)-6-fluoro-1-oxoisoindoline-5-carboxylic acid (400 mg, 0.826 mmol, 1 equiv) in 5 mL DMF was added Pd/C (0.3 g, Palladium 10% on Carbon, wetted with ca.55% water) under nitrogen atmosphere in a 100 mL round-bottom flask. The mixture was hydrogenated at room temperature for overnight under hydrogen atmosphere using a hydrogen balloon, filtered through a Celite pad and concentrated under reduced pressure. The resulting mixture was diluted with water (100 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by reverse-phase flash chromatography to afford 2-(2,6-dioxopiperidin-3-yl)-6-fluoro-1-oxoisoindoline-5-carboxylic acid (107 mg, 42%) as an off-white solid.

Step 5. Preparation of N-(benzo[b]thiophen-3-yl)-2-(2,6-dioxopiperidin-3-yl)-6-fluoro-1-oxoisoindoline-5-carboxamide. To a stirred solution of 2-(2,6-dioxopiperidin-3-yl)-6-fluoro-1-oxoisoindoline-5-carboxylic acid (107 mg, 0.349 mmol, 1 equiv), 1-benzothiophen-3-amine (52.1 mg, 0.349 mmol, 1 equiv) and DIEA (135.4 mg, 1.047 mmol, 3 equiv) in DMF (1 mL) was added HATU (199.2 mg, 0.523 mmol, 1.5 equiv) in portions at room temperature under nitrogen atmosphere. The resulting mixture was stirred for overnight at room temperature under nitrogen atmosphere. The resulting mixture was diluted with water (50 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product was purified by Prep-HPLC to afford N-(benzo[b]thiophen-3-yl)-2-(2,6-dioxopiperidin-3-yl)-6-fluoro-1-oxoisoindoline-5-carboxamide (5 mg, 3%) as an off-white solid.

LC-MS (ES, m/z)=438 [M+1]⁺. ¹H NMR (400 MHz, DMSO-d₆) δ 11.03 (s, 1H), 10.80 (s, 1H), 8.14 (d, J=7.9 Hz, 2H), 8.06-7.95 (m, 2H), 7.72 (d, J=8.7 Hz, 1H), 7.50-7.40 (m, 2H), 5.17 (dd, J=13.3, 5.1 Hz, 1H), 4.57-4.36 (m, 2H), 2.93 (ddd, J=18.1, 13.5, 5.4 Hz, 1H), 2.62 (d, J=17.3 Hz, 1H), 2.47-2.37 (m, 1H), 2.11-2.01 (m, 1H), 1.24 (s, 2H).

### Example 92

### Synthesis of 3-(5-(4,6-difluoroindoline-1-carbonyl)-1-oxoisoindolin-2-yl)piperidine-2,6-dione

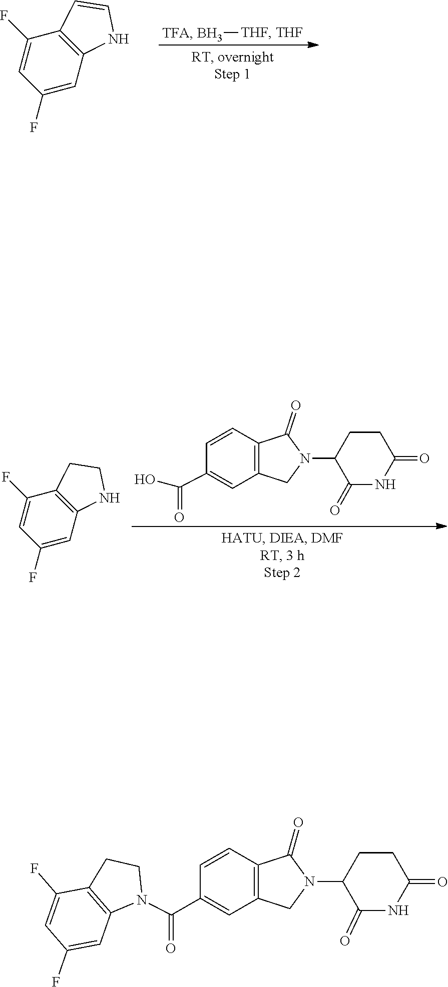

Step 1. Preparation of 4,6-difluoroindoline. A solution of 4,6-difluoro-1H-indole (500 mg, 3.265 mmol, 1 equiv) in THF (10.00 mL, 123.417 mmol, 37.80 equiv) was treated with BH₃-THF (9.80 mL, 9.795 mmol, 3 equiv) for 30 min at 0° C. under nitrogen atmosphere followed by the addition of TFA (1.86 g, 16.325 mmol, 5 equiv) dropwise at 0° C. The resulting mixture was stirred for overnight at room temperature under nitrogen atmosphere. The reaction was quenched with Water at 0° C. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reverse-phase flash chromatography to afford 4,6-difluoroindoline (200 mg, 39%) as a yellow oil.

Step 2. Preparation of 3-(5-(4,6-difluoroindoline-1-carbonyl)-1-oxoisoindolin-2-yl)piperidine-2,6-dione. To a stirred mixture of 4,6-difluoro-2,3-dihydro-1H-indole (161.4 mg, 1.041 mmol, 1.5 equiv) and 2-(2,6-dioxopiperidin-3-yl)-1-oxo-3H-isoindole-5-carboxylic acid (200 mg, 0.694 mmol, 1.00 equiv) in DMF (3 mL, 38.765 mmol, 55.87 equiv) was added DIEA (269.0 mg, 2.082 mmol, 3 equiv) and HATU (395.7 mg, 1.041 mmol, 1.5 equiv) at room temperature. The resulting mixture was stirred for 3 h at room temperature. The resulting mixture was extracted with EtOAc (3×30 mL). The combined organic layers were washed with brine (2×20 mL), dried over anhydrous Na₂SO₄. After filtration, the filtrate was concentrated under reduced pressure. The residue was purified by Prep-HPLC to afford 3-(5-(4,6-difluoroindoline-1-carbonyl)-1-oxoisoindolin-2-yl)piperidine-2,6-dione (6.5 mg, 2.2%) as a white solid.

LC-MS (ES, m/z)=426.15 [M+1]⁺. ¹H NMR (400 MHz, DMSO-d₆) δ 11.02 (s, 1H), 7.85 (d, J=7.6 Hz, 2H), 7.74 (d, J=7.9 Hz, 2H), 6.97 (t, J=9.6 Hz, 1H), 5.16 (dd, J=13.3, 5.1 Hz, 1H), 4.54 (d, J=17.6 Hz, 1H), 4.41 (d, J=17.6 Hz, 1H), 4.10 (t, J=8.3 Hz, 2H), 3.09 (t, J=8.2 Hz, 2H), 2.97-2.90 (m, 1H), 2.61 (d, J=17.2 Hz, 1H), 2.40 (dd, J=13.3, 4.6 Hz, 1H), 2.08-2.00 (m, 1H), 1.24 (s, 1H).

### Example 93

### Synthesis of 2-(2,6-dioxopiperidin-3-yl)-1-oxo-N-phenylisoindoline-5-carboxamide

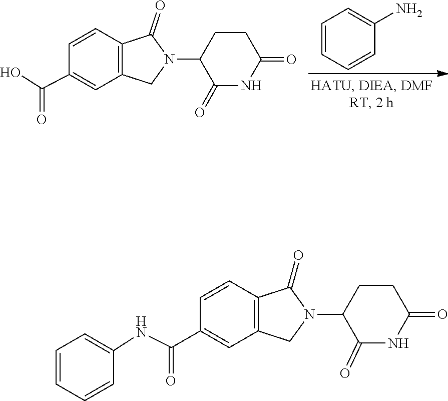

Step 1. Preparation of 2-(2,6-dioxopiperidin-3-yl)-1-oxo-N-phenylisoindoline-5-carboxamide. To a stirred mixture of aniline (25 mg, 0.268 mmol, 1 equiv) and 2-(2,6-dioxopiperidin-3-yl)-1-oxo-3H-isoindole-5-carboxylic acid (92.8 mg, 0.322 mmol, 1.2 equiv) in DMF (1 ml) were added DIEA (138.7 mg, 1.072 mmol, 4 equiv) and HATU (153.1 mg, 0.402 mmol, 1.5 equiv) room temperature. The resulting mixture was stirred for additional 2 h at room temperature. The residue was diluted with water (10 mL). The resulting mixture was extracted with EtOAc (3×20 mL). The combined organic layers were washed with water (2×20 mL), dried over anhydrous Na₂SO₄. After filtration, the filtrate was concentrated under reduced pressure. The residue was purified by reverse-phase flash chromatography to afford 2-(2,6-dioxopiperidin-3-yl)-1-oxo-N-phenylisoindoline-5-carboxamide (90 mg, 88%) as a light-yellow solid.

LC-MS (ES, m/z)=362 [M−1]⁻. ¹H NMR (400 MHz, DMSO-d₆) δ 11.02 (s, 1H), 10.44 (s, 1H), 8.17 (s, 1H), 8.09 (dd, J=7.9, 1.5 Hz, 1H), 7.88 (d, J=7.9 Hz, 1H), 7.83-7.76 (m, 2H), 7.37 (t, J=7.9 Hz, 2H), 7.13 (tt, J=7.3, 1.2 Hz, 1H), 5.16 (dd, J=13.3, 5.1 Hz, 1H), 4.56 (d, J=17.5 Hz, 1H), 4.44 (d, J=17.5 Hz, 1H), 2.93 (ddd, J=17.3, 13.5, 5.4 Hz, 1H), 2.77 (d, J=4.5 Hz, 1H), 2.62 (ddd, J=17.3, 4.4, 2.3 Hz, 1H), 2.43 (td, J=13.3, 4.6 Hz, 1H).

### Example 94

### Synthesis of 2-(2,6-dioxopiperidin-3-yl)-N-(2-fluorophenyl)-1-oxoisoindoline-5-carboxamide

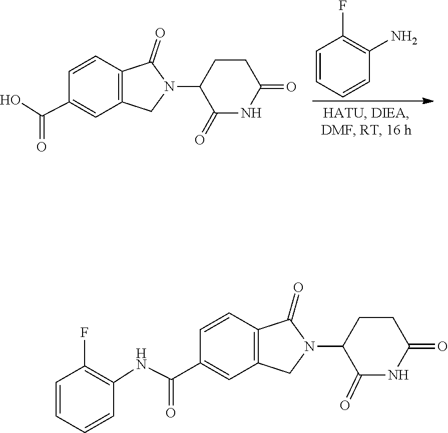

Step 1. Preparation of 2-(2,6-dioxopiperidin-3-yl)-N-(2-fluorophenyl)-1-oxoisoindoline-5-carboxamide. To a stirred solution of 2-(2,6-dioxopiperidin-3-yl)-1-oxoisoindole-5-carboxylic acid (100 mg, 0.347 mmol, 1 equiv), 2-fluoroaniline (46.3 mg, 0.416 mmol, 1.2 equiv) and DIEA (179.4 mg, 1.388 mmol, 4 equiv) in DMF (1 mL) was added HATU (197.9 mg, 0.520 mmol, 1.5 equiv) in portions at room temperature under nitrogen atmosphere. The resulting mixture was stirred for overnight at room temperature under nitrogen atmosphere. The resulting mixture was diluted with water (20 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product (80 mg) was purified by Prep-HPLC to afford 2-(2,6-dioxopiperidin-3-yl)-N-(2-fluorophenyl)-1-oxoisoindoline-5-carboxamide (9 mg, 7%) as an off-white solid.

LC-MS (ES, m/z)=382 [M+1]⁺. ¹H NMR (400 MHz, DMSO-d₆) δ 11.02 (s, 1H), 10.33 (d, J=2.6 Hz, 1H), 8.19 (s, 1H), 8.10 (d, J=7.9 Hz, 1H), 7.88 (d, J=7.9 Hz, 1H), 7.63 (t, J=7.8 Hz, 1H), 7.37-7.20 (m, 3H), 5.17 (dd, J=13.2, 5.1 Hz, 1H), 4.61-4.41 (m, 2H), 2.93 (ddd, J=18.0, 13.4, 5.4 Hz, 1H), 2.62 (d, J=17.0 Hz, 1H), 2.43 (dt, J=14.4, 7.2 Hz, 1H), 2.10-2.01 (m, 1H), 1.24 (s, 1H).

### Example 95

### Synthesis of 2-(2,6-dioxopiperidin-3-yl)-1-oxo-N-(quinolin-8-yl)isoindoline-5-carboxamide

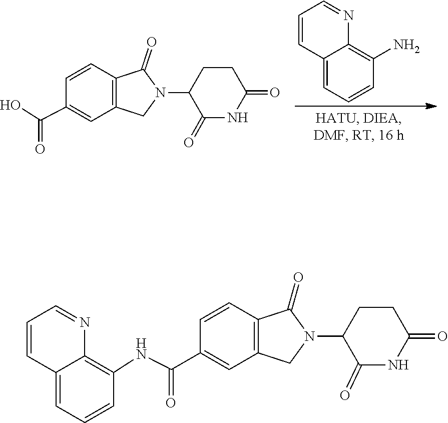 [SMILES: O=C1CCC(N2Cc3cc(C(=O)O)ccc3C2=O)C(=O)N1 ; Nc1cccc2cccnc12 ; O=C1CCC(N2Cc3cc(C(=O)Nc4cccc5cccnc45)ccc3C2=O)C(=O)N1]

Step 1. Preparation of 2-(2,6-dioxopiperidin-3-yl)-1-oxo-N-(quinolin-8-yl)isoindoline-5-carboxamide. To a stirred solution of 2-(2,6-dioxopiperidin-3-yl)-1-oxoisoindole-5-carboxylic acid (100 mg, 0.347 mmol, 1 equiv), 8-aminoquinoline (60.0 mg, 0.416 mmol, 1.2 equiv) and DIEA (179.4 mg, 1.388 mmol, 4 equiv) in DMF (1 mL) was added HATU (197.9 mg, 0.520 mmol, 1.5 equiv) in portions at room temperature under nitrogen atmosphere. The resulting mixture was stirred for overnight at room temperature under nitrogen atmosphere. The resulting mixture was diluted with water (50 mL). The resulting mixture was extracted with EtOAc (3×20 mL). The combined organic layers were washed with brine (2×30 mL), dried over anhydrous Na₂SO₄. After filtration, the filtrate was concentrated under reduced pressure. The crude product (80 mg) was purified by Prep-HPLC to afford 2-(2,6-dioxopiperidin-3-yl)-1-oxo-N-(quinolin-8-yl)isoindoline-5-carboxamide (21 mg, 15%) as an off-white solid.

LC-MS (ES, m/z)=415.05 [M+1]⁺. ¹H NMR (400 MHz, DMSO-d₆) δ 11.04 (s, 1H), 10.76 (s, 1H), 9.00 (dd, J=4.2, 1.6 Hz, 1H), 8.75 (dd, J=7.6, 1.3 Hz, 1H), 8.49 (dd, J=8.3, 1.7 Hz, 1H), 8.32-8.27 (m, 1H), 8.17 (dd, J=7.9, 1.5 Hz, 1H), 7.97 (d, J=7.9 Hz, 1H), 7.79 (dd, J=8.3, 1.4 Hz, 1H), 7.74-7.65 (m, 2H), 5.18 (dd, J=13.3, 5.1 Hz, 1H), 4.67-4.46 (m, 2H), 2.94 (ddd, J=18.1, 13.6, 5.4 Hz, 1H), 2.63 (d, J=17.9 Hz, 1H), 2.43 (td, J=13.2, 4.5 Hz, 2H), 2.10-2.02 (m, 1H).

### Example 96

### Synthesis of N-cyclohexyl-2-(2,6-dioxopiperidin-3-yl)-1-oxoisoindoline-5-carboxamide

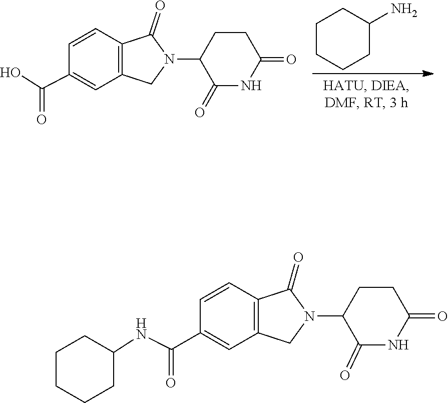

Step 1. Preparation of N-cyclohexyl-2-(2,6-dioxopiperidin-3-yl)-1-oxoisoindoline-5-carboxamide. To a stirred mixture of cyclohexylamine (50 mg, 0.504 mmol, 1 equiv) and 2-(2,6-dioxopiperidin-3-yl)-1-oxo-3H-isoindole-5-carboxylic acid (174.3 mg, 0.605 mmol, 1.2 equiv) in DMF (1.5 ml) were added DIEA (260.6 mg, 2.016 mmol, 4 equiv) and HATU (287.5 mg, 0.756 mmol, 1.5 equiv) at room temperature. The resulting mixture was stirred for additional 3 h at room temperature. The residue was diluted with water (10 mL). The resulting mixture was extracted with EtOAc (3×20 mL). The combined organic layers were washed with water (2×10 mL), dried over anhydrous Na₂SO₄. After filtration, the filtrate was concentrated under reduced pressure. The residue was purified by reverse-phase flash chromatography to afford N-cyclohexyl-2-(2,6-dioxopiperidin-3-yl)-1-oxoisoindoline-5-carboxamide (46 mg, 23%) as a white solid. LC-MS (ES, m/z)=370 [M+1]⁺. ¹H NMR (400 MHz, DMSO-d₆) δ 11.00 (s, 1H), 8.39 (d, J=7.9 Hz, 1H), 8.04 (s, 1H), 7.96 (d, J=8.0 Hz, 1H), 7.79 (d, J=7.9 Hz, 1H), 5.14 (dd, J=13.3, 5.1 Hz, 1H), 4.51 (d, J=17.4 Hz, 1H), 4.39 (d, J=17.4 Hz, 1H), 3.78 (s, 1H), 2.99-2.85 (m, 1H), 2.59 (s, 1H), 2.06-1.98 (m, 1H), 1.84 (s, 2H), 1.75 (s, 2H), 1.62 (d, J=12.6 Hz, 1H), 1.32 (s, 2H), 1.29 (s, 1H), 1.24 (s, 1H), 1.14 (s, 1H).

### Example 97

### Synthesis of 2-(2,6-dioxopiperidin-3-yl)-N-(2-hydroxyphenyl)-1-oxoisoindoline-5-carboxamide

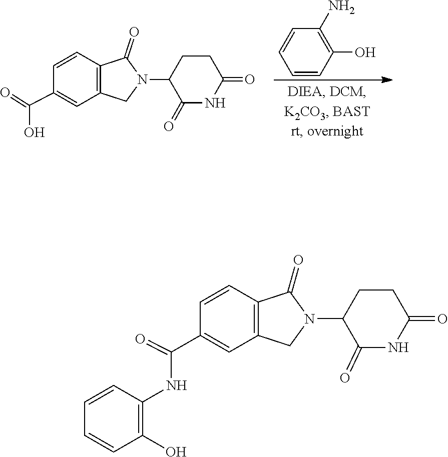

Step 1. Preparation of 2-(2,6-dioxopiperidin-3-yl)-N-(2-hydroxyphenyl)-1-oxoisoindoline-5-carboxamide. A solution of 2-(2,6-dioxopiperidin-3-yl)-1-oxo-3H-isoindole-5-carboxylic acid (500 mg, 1.735 mmol, 1 equiv) and 2-aminophenol (189.2 mg, 1.735 mmol, 1 equiv) in DCM (10 mL, 157.306 mmol, 90.69 equiv) was treated with DIEA (672.5 mg, 5.205 mmol, 3 equiv) and K₂CO₃ (4.79 g, 34.700 mmol, 20 equiv) for 30 min at room temperature under nitrogen atmosphere followed by the addition of BAST (0.96 g, 4.338 mmol, 2.5 equiv) at 0° C. The resulting mixture was stirred for overnight at room temperature under nitrogen atmosphere. The reaction was quenched by the addition of Water/Ice (10 mL) at 0° C. The resulting mixture was extracted with EtOAc (3×50 mL). The combined organic layers were washed with brine (2×30 mL), dried over anhydrous Na₂SO₄. After filtration, the filtrate was concentrated under reduced pressure. The crude product was purified by Prep-HPLC to afford 2-(2,6-dioxopiperidin-3-yl)-N-(2-hydroxyphenyl)-1-oxoisoindoline-5-carboxamide (20 mg, 3%) as a white solid.

LC-MS (ES, m/z)=380.05 [M+1]⁺. ¹H NMR (400 MHz, DMSO-d₆) δ 11.02 (s, 1H), 9.74 (s, 1H), 9.68 (s, 1H), 8.18 (s, 1H), 8.09 (d, J=7.9 Hz, 1H), 7.87 (d, J=7.9 Hz, 1H), 7.68 (d, J=7.9 Hz, 1H), 7.10-7.01 (m, 1H), 6.94 (dd, J=8.0, 1.4 Hz, 1H), 6.85 (t, J=7.6 Hz, 1H), 5.16 (dd, J=13.2, 5.1 Hz, 1H), 4.56 (d, J=17.7 Hz, 1H), 4.43 (d, J=17.5 Hz, 1H), 2.98-2.87 (m, 1H), 2.62 (d, J=17.1 Hz, 1H), 2.42 (d, J=4.4 Hz, 1H), 2.05 (dd, J=11.6, 5.6 Hz, 1H).

### Example 98

### Synthesis of 3-(5-(2-oxa-5-azabicyclo[2.2.1]heptane-5-carbonyl)-1-oxoisoindolin-2-yl)piperidine-2,6-dione

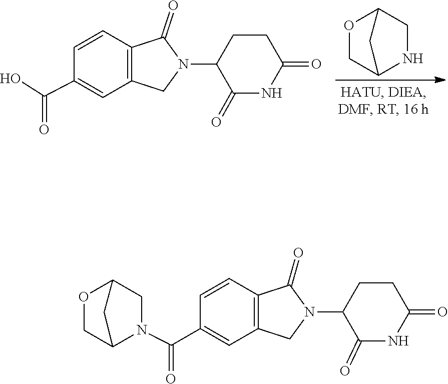

Step 1. Preparation of 3-(5-(2-oxa-5-azabicyclo[2.2.1]heptane-5-carbonyl)-1-oxoisoindolin-2-yl)piperidine-2,6-dione. To a stirred solution of 2-(2,6-dioxopiperidin-3-yl)-1-oxoisoindole-5-carboxylic acid (100 mg, 0.347 mmol, 1 equiv), 2-oxa-5-azabicyclo[2.2.1]heptane (68.7 mg, 0.694 mmol, 2 equiv) and DIEA (134.5 mg, 1.041 mmol, 3 equiv) in DMF (2 mL) was added HATU (197.8 mg, 0.520 mmol, 1.5 equiv) in portions at room temperature under nitrogen atmosphere. The resulting mixture was stirred for overnight at room temperature under nitrogen atmosphere. The resulting mixture was diluted with water (50 mL) and extracted with EtOAc (3×20 mL). The combined organic layers were washed with brine (2×20 mL), dried over anhydrous Na₂SO₄. After filtration, the filtrate was concentrated under reduced pressure. The crude product (100 mg) was purified by Prep-HPLC to afford 3-(5-(2-oxa-5-azabicyclo[2.2.1]heptane-5-carbonyl)-1-oxoisoindolin-2-yl)piperidine-2,6-dione (21 mg, 16%) as an off-white solid.

LC-MS (ES, m/z)=370.05 [M+1]⁺. ¹H NMR (400 MHz, DMSO-d₆) δ 11.00 (s, 1H), 7.83-7.70 (m, 2H), 7.68-7.57 (m, 1H), 5.14 (dt, J=13.4, 4.3 Hz, 1H), 4.68 (s, 1H), 4.59-4.44 (m, 2H), 4.43-4.37 (m, 1H), 4.36 (s, 1H), 3.92-3.75 (m, 2H), 3.67 (dd, J=7.5, 1.7 Hz, 1H), 3.51 (t, J=9.6 Hz, 1H), 3.23 (d, J=10.0 Hz, 1H), 2.98-2.85 (m, 1H), 2.61 (d, J=17.2 Hz, 1H), 2.48-2.34 (m, 1H), 2.10-1.98 (m, 1H), 1.93-1.73 (m, 2H).

### Example 99

### Synthesis of 3-(5-(5-methyl-2,5-diazabicyclo[2.2.1]heptane-2-carbonyl)-1-oxoisoindolin-2-yl)piperidine-2,6-dione

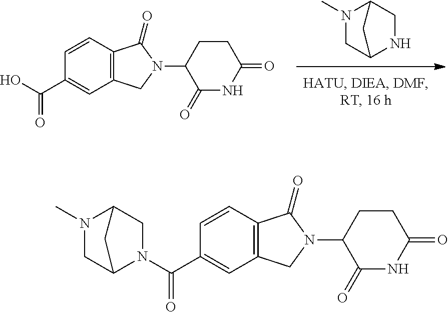

Step 1. Preparation of 3-(5-(5-methyl-2,5-diazabicyclo[2.2.1]heptane-2-carbonyl)-1-oxoisoindolin-2-yl)piperidine-2,6-dione. To a stirred solution of 2-(2,6-dioxopiperidin-3-yl)-1-oxoisoindole-5-carboxylic acid (100 mg, 0.347 mmol, 1 equiv), 2-methyl-2,5-diazabicyclo[2.2.1]heptane (77.8 mg, 0.694 mmol, 2 equiv) and DIEA (134.5 mg, 1.041 mmol, 3 equiv) in DMF (2 mL) was added HATU (197.8 mg, 0.520 mmol, 1.5 equiv) in portions at room temperature under nitrogen atmosphere. The resulting mixture was stirred for overnight at room temperature under nitrogen atmosphere. The resulting mixture was diluted with water (50 mL) and extracted with EtOAc (3×20 mL). The combined organic layers were washed with brine (2×20 mL), dried over anhydrous Na₂SO₄. After filtration, the filtrate was concentrated under reduced pressure. The crude product was purified by Prep-HPLC to afford 3-(5-(5-methyl-2,5-diazabicyclo[2.2.1]heptane-2-carbonyl)-1-oxoisoindolin-2-yl)piperidine-2,6-dione (7.9 mg, 6%) as an off-white solid. LC-MS (ES, m/z)=383.05 [M+1]⁺. ¹H NMR (400 MHz, Methanol-d₄) δ 8.49 (s, 1H), 7.94-7.54 (m, 3H), 5.16 (dd, J=13.3, 5.0 Hz, 1H), 4.58-4.44 (m, 2H), 4.22 (d, J=37.6 Hz, 1H), 3.89-3.59 (m, 3H), 3.21 (t, J=12.2 Hz, 1H), 3.09-2.93 (m, 1H), 2.92-2.73 (m, 5H), 2.49 (qt, J=13.3, 3.8 Hz, 1H), 2.37-2.11 (m, 3H), 1.39-1.26 (m, 1H).

### Example 100

### Synthesis of N-(6,6-difluorospiro[3.3]heptan-2-yl)-2-(2,6-dioxopiperidin-3-yl)-1-oxoisoindoline-5-carboxamide

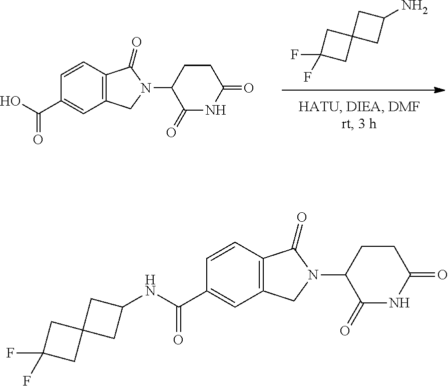 [SMILES: O=C1CCC(N2Cc3cc(C(=O)O)ccc3C2=O)C(=O)N1 ; NC1CC2(C1)CC(F)(F)C2 ; O=C1CCC(N2Cc3cc(C(=O)NC4CC5(C4)CC(F)(F)C5)ccc3C2=O)C(=O)N1]

Step 1. Preparation of N-(6,6-difluorospiro[3.3]heptan-2-yl)-2-(2,6-dioxopiperidin-3-yl)-1-oxoisoindoline-5-carboxamide. To a stirred mixture of 2-(2,6-dioxopiperidin-3-yl)-1-oxoisoindole-5-carboxlic acid (200 m 0.694 mmol, 1 equiv) and 6,6-difluorospiro[3.3]heptan-2-amine (102.1 mg, 0.694 mmol, 1 equiv) in DMF (4.00 mL, 51.703 mmol, 74.50 equiv) were added DIEA (269.0 mg, 2.082 mmol, 3 equiv) and HATU (395.7 mg, 1.041 mmol, 1.5 equiv) at room temperature. The resulting mixture was stirred for 3 h at room temperature. The resulting mixture was diluted with water and extracted with EtOAc (3×20 mL). The combined organic layers were washed with brine (2×20 mL), dried over anhydrous Na₂SO₄. After filtration, the filtrate was concentrated under reduced pressure. The crude product was purified by Prep-HPLC to afford N-(6,6-difluorospiro[3.3]heptan-2-yl)-2-(2,6-dioxopiperidin-3-yl)-1-oxoisoindoline-5-carboxamide (31 mg, 11%) as a white solid.

LC-MS (ES, m/z)=418.30 [M+1]⁺. ¹H NMR (400 MHz, DMSO-d₆) δ 11.00 (s, 1H), 8.81 (d, J=7.3 Hz, 1H), 8.05 (s, 1H), 7.96 (dd, J=7.9, 1.5 Hz, 1H), 7.80 (d, J=7.9 Hz, 1H), 5.14 (dd, J=13.3, 5.1 Hz, 1H), 4.51 (d, J=17.5 Hz, 1H), 4.46-4.32 (m, 2H), 2.92 (ddd, J=17.2, 13.6, 5.4 Hz, 1H), 2.76-2.53 (m, 5H), 2.49-2.34 (m, 3H), 2.26 (ddt, J=11.1, 8.4, 2.1 Hz, 2H), 2.02 (dtd, J=12.5, 5.3, 2.2 Hz, 1H).

### Example 101

### Synthesis of 3-(5-(3,3-dimethylindoline-1-carbonyl)-1-oxoisoindolin-2-yl)piperidine-2,6-dione

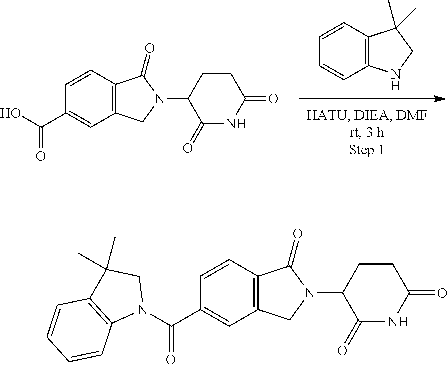 [SMILES: O=C1CCC(N2Cc3cc(C(=O)O)ccc3C2=O)C(=O)N1 ; CC1(C)CNc2ccccc21 ; CC1(C)CN(C(=O)c2ccc3c(c2)CN(C2CCC(=O)NC2=O)C3=O)c2ccccc21]

Step 1. Preparation of 3-(5-(3,3-dimethylindoline-1-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (200 mg, 0.694 mmol, 1 equiv) and 3,3-dimethyl-1,2-dihydroindole (102.1 mg, 0.694 mmol, 1 equiv) in DMF (4.00 mL) were added DIEA (269.0 mg, 2.082 mmol, 3 equiv) and HATU (395.7 mg, 1.041 mmol, 1.5 equiv) at room temperature. The resulting mixture was stirred for 3 h at room temperature. The resulting mixture was diluted with water and extracted with EtOAc (3×30 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(5-(3,3-dimethylindoline-1-carbonyl)-1-oxoisoindolin-2-yl)piperidine-2,6-dione (26.5 mg, 8.61%) as a white solid. LC-MS (ES, m/z)=418.05 [M+1]⁺. ¹H NMR (400 MHz, DMSO-d₆) S S 11.01 (s, 1H), 8.07 (s, 1H), 7.87-7.81 (m, 2H), 7.71 (d, J=7.8 Hz, 1H), 7.30 (d, J=7.4 Hz, 1H), 7.23 (s, 1H), 7.11 (s, 1H), 5.16 (dd, J=13.3, 5.1 Hz, 1H), 4.58-4.40 (m, 2H), 3.76 (s, 2H), 2.98-2.88 (m, 1H), 2.62 (d, J=17.0 Hz, 1H), 2.45-2.38 (m, 1H), 2.08-2.00 (m, 1H), 1.25 (d, J=10.5 Hz, 7H).

### Example 102

### Synthesis of 1-(7-(6-fluoroindoline-1-carbonyl)naphthalen-2-yl)dihydropyrimidine-2,4(1H,3H)-dione

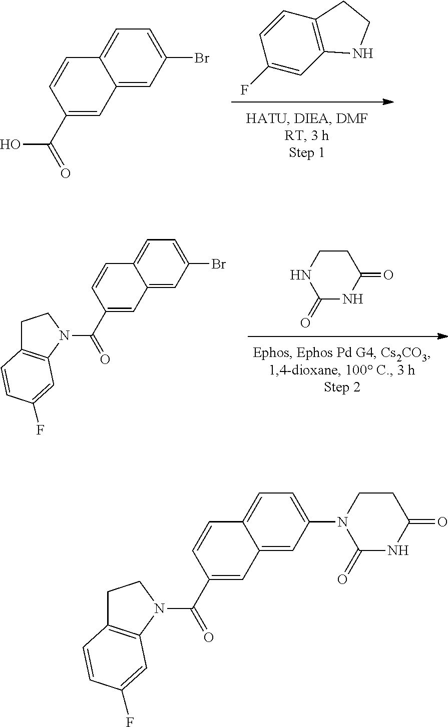 [SMILES: O=C(O)c1ccc2ccc(Br)cc2c1 ; Fc1ccc2c(c1)NCC2 ; O=C(c1ccc2ccc(Br)cc2c1)N1CCc2ccc(F)cc21 ; O=C1CCNC(=O)N1 ; O=C1CCN(c2ccc3ccc(C(=O)N4CCc5ccc(F)cc54)cc3c2)C(=O)N1]

Step 1. Preparation of (7-bromonaphthalen-2-yl)(6-fluoroindolin-1-yl)methanone. To a stirred mixture of 7-bromonaphthalene-2-carboxylic acid (500.0 mg, 1.991 mmol, 1 equiv), 6-fluoro-2,3-dihydro-1H-indole (327.7 mg, 2.389 mmol, 1.2 equiv) and DIEA (772.1 mg, 5.973 mmol, 3 equiv) in DMF (10 mL) was added HATU (1135.8 mg, 2.987 mmol, 1.5 equiv) at room temperature under air atmosphere. The resulting mixture was stirred for 3 h at room temperature under the air atmosphere. The resulting mixture was diluted with water and extracted with EtOAc (3×50 mL). The combined organic layers were washed with water (2×30 mL), dried over anhydrous Na₂SO₄. After filtration, the filtrate was concentrated under reduced pressure. The residue was purified by reversed-phase flash chromatography to afford (7-bromonaphthalen-2-yl)(6-fluoroindolin-1-yl)methanone (600 mg, 80%) as a white solid. LC-MS (ES, m/z)=369.90 [M+1]⁺.

Step 2. Preparation of 1-(7-(6-fluoroindoline-1-carbonyl)naphthalen-2-yl)dihydropyrimidine-2,4(1H,3H)-dione. To a stirred of (7-bromonaphthalen-2-yl)(6-fluoroindolin-1-yl)methanone (300.0 mg, 0.810 mmol, 1 equiv), Ephos (43.3 mg, 0.081 mmol, 0.1 equiv), Ephos Pd G₄ (74.4 mg, 0.081 mmol, 0.1 equiv), Cs₂CO₃ (792.0 mg, 2.430 mmol, 3 equiv) in 1,4-dioxane (6 mL) was added 5,6-dihydrouracil (110.9 mg, 0.972 mmol, 1.2 equiv) at room temperature under air atmosphere. The resulting mixture was stirred for 3 h at 100° C. under nitrogen atmosphere. The resulting mixture was diluted with water and extracted with EtOAc (3×50 mL). The combined organic layers were washed with water (3×50 mL), dried over anhydrous Na₂SO₄. After filtration, the filtrate was concentrated under reduced pressure. The residue was purified by reversed-phase flash chromatography to afford 1-(7-(6-fluoroindoline-1-carbonyl)naphthalen-2-yl)dihydropyrimidine-2,4(1H,3H)-dione (127 mg, 38%) as a white solid. LC-MS (ES, m/z)=404.05 [M+1]⁺. ¹H NMR (400 MHz, DMSO-d₆) δ 10.47 (s, 1H), 8.19 (s, 1H), 8.02 (dd, J=11.9, 8.7 Hz, 2H), 7.93 (d, J=2.1 Hz, 2H), 7.67 (ddd, J=8.6, 5.9, 1.9 Hz, 2H), 7.30 (dd, J=8.2, 5.9 Hz, 1H), 6.89 (t, J=9.2 Hz, 1H), 4.14 (t, J=8.3 Hz, 2H), 3.94 (t, J=6.6 Hz, 2H), 3.08 (t, J=8.3 Hz, 2H), 2.77 (t, J=6.6 Hz, 2H).

### Example 103

### Synthesis of 3-(5-(4,6-difluoroisoindoline-2-carbonyl)-1-oxoisoindolin-2-yl)piperidine-2,6-dione

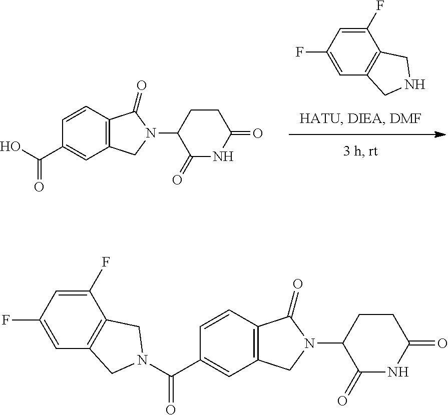 [SMILES: O=C1CCC(N2Cc3cc(C(=O)O)ccc3C2=O)C(=O)N1 ; Fc1cc(F)c2c(c1)CNC2 ; O=C1CCC(N2Cc3cc(C(=O)N4Cc5cc(F)cc(F)c5C4)ccc3C2=O)C(=O)N1]

Step 1. Preparation of 3-(5-(4,6-difluoroisoindoline-2-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (92.90 mg, 0.322 mmol, 1 equiv) and 4,6-difluoro-2,3-dihydro-1H-isoindole (50 mg, 0.322 mmol, 1.00 equiv) in DMF (1.5 mL) were added DIEA (125 mg, 0.966 mmol, 3 equiv) and HATU (183.8 mg, 0.483 mmol, 1.5 equiv) at room temperature. The resulting mixture was stirred for 3 h at room temperature. The resulting mixture was diluted with water (20 mL) and extracted with EtOAc (3×20 mL). The combined organic layers were washed with brine (2×30 mL), dried over anhydrous Na₂SO₄. After filtration, the filtrate was concentrated under reduced pressure. The crude product was purified by Prep-HPLC to afford 3-(5-(4,6-difluoroisoindoline-2-carbonyl)-1-oxoisoindolin-2-yl)piperidine-2,6-dione (20 mg, 14%) as a white solid. LC-MS (ES, m/z)=425.95 [M+1]⁺. ¹H NMR (400 MHz, DMSO-d₆) δ 11.00 (s, 1H), 7.87-7.80 (m, 2H), 7.76-7.69 (m, 1H), 7.23-7.04 (m, 2H), 5.15 (dd, J=13.2, 5.1 Hz, 1H), 4.91 (d, J=12.6 Hz, 2H), 4.80 (s, 2H), 4.54 (dd, J=17.5, 5.4 Hz, 1H), 4.41 (dd, J=17.7, 4.6 Hz, 1H), 3.00-2.86 (m, 1H), 2.61 (d, J=17.4 Hz, 1H), 2.41 (t, J=13.8 Hz, 1H), 2.08-2.00 (m, 1H).

### Example 104

### Synthesis of 3-(5-(5-chloro-1,2,3,4-tetrahydroisoquinoline-2-carbonyl)-1-oxoisoindolin-2-yl)piperidine-2,6-dione

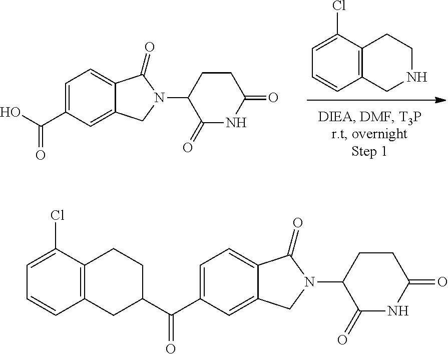 [SMILES: O=C1CCC(N2Cc3cc(C(=O)O)ccc3C2=O)C(=O)N1 ; Clc1cccc2c1CCNC2 ; O=C1CCC(N2Cc3cc(C(=O)C4CCc5c(Cl)cccc5C4)ccc3C2=O)C(=O)N1]

Step 1. Preparation of 3-(5-(5-chloro-1,2,3,4-tetrahydroisoquinoline-2-carbonyl)-1-oxoisoindolin-2-yl)piperidine-2,6-dione. To a stirred solution of 2-(2,6-dioxopiperidin-3-yl)-1-oxo-3H-isoindole-5-carboxylic acid (200.0 mg, 0.694 mmol, 1 equiv) and 5-chloro-1,2,3,4-tetrahydroisoquinoline (174.5 mg, 1.041 mmol, 1.5 equiv) in DMF (3 mL) was added DIEA (0.36 mL, 2.082 mmol, 3 equiv) and T₃P (883.1 mg, 1.388 mmol, 2 equiv, 50% in EA) at room temperature. The resulting mixture was stirred overnight at room temperature. The resulting mixture was diluted with water (50 mL). The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford 3-(5-(5-chloro-1,2,3,4-tetrahydroisoquinoline-2-carbonyl)-1-oxoisoindolin-2-yl)piperidine-2,6-dione (150 mg, 49%) as an off-white solid. LC-MS (ES, m/z)=438.15 [M+1]⁺. ¹H NMR (400 MHz, DMSO-d₆) δ 11.01 (s, 1H), 7.81 (d, J=7.8 Hz, 1H), 7.72 (s, 2H), 7.59-7.29 (s, 3H), 5.15 (dd, J=13.3, 5.1 Hz, 1H), 4.83 (s, 1H), 4.52 (d, J=17.6 Hz, 2H), 4.39 (d, J=17.5 Hz, 1H), 3.90 (s, 1H), 3.65 (1H), 2.91 (ddd, J=27.8, 14.4, 7.1 Hz, 3H), 2.66-2.57 (m, 1H), 2.42 (qd, J=13.2, 4.4 Hz, 1H), 2.07-1.99 (m, 1H).

### Example 105

### Synthesis of 3-(5-(6′-fluorospiro[cyclopropane-1,3′-indoline]-1′-carbonyl)-1-oxoisoindolin-2-yl)piperidine-2,6-dione

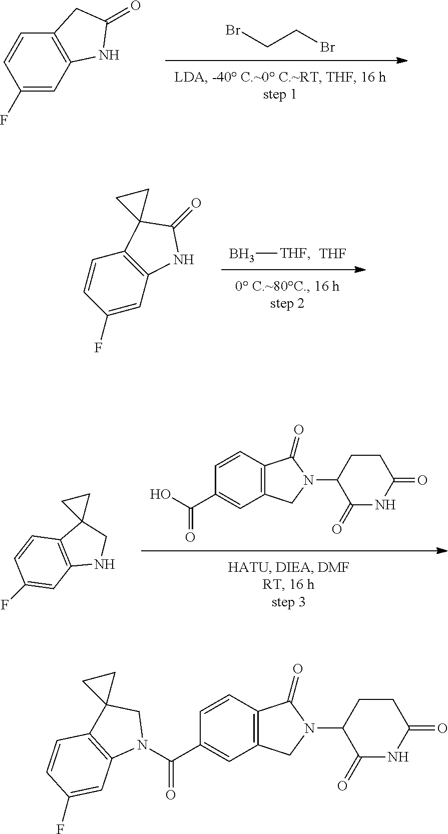

Step 1. Preparation of 6′-fluorospiro[cyclopropane-1,3′-indolin]-2′-one. To a stirred solution of 6-fluoro-1,3-dihydroindol-2-one (1 g, 6.616 mmol, 1 equiv) in THF (10 mL) was added LDA (2M in THF) (2.8 g, 26.464 mmol, 4 equiv) dropwise at −40° C. under nitrogen atmosphere. The resulting mixture was stirred for 30 min at −40° C. under nitrogen atmosphere. To the above mixture was added dibromoethane (3.7 g, 19.848 mmol, 3 equiv) dropwise over 10 min at 0° C. The resulting mixture was stirred for additional overnight at room temperature. The reaction was quenched with sat. NH₄Cl (aq.) at 0° C. The resulting mixture was diluted with water (100 mL). The resulting mixture was extracted with EtOAc (3×200 mL). The combined organic layers were washed with brine (2×200 mL), dried over anhydrous Na₂SO₄. After filtration, the filtrate was concentrated under reduced pressure. The residue was purified by reversed-phase flash chromatography to afford 6′-fluorospiro[cyclopropane-1,3′-indolin]-2′-one (500 mg, 43%) as a yellow solid. LC-MS (ES, m/z)=178.25 [M+1]⁺.

Step 2. Preparation of 6′-fluorospiro[cyclopropane-1,3′-indoline]. To a stirred solution of 6′-fluorospiro[cyclopropane-1,3′-indolin]-2′-one (500 mg, 2.822 mmol, 1 equiv) in THF (5 mL) was added BH₃-THF (8.47 mL, 8.466 mmol, 3 equiv) at 0° C. under nitrogen atmosphere. The resulting mixture was stirred overnight at 80° C. under nitrogen atmosphere. The mixture was allowed to cool down to room temperature. The reaction was quenched by the addition of MeOH (20 mL) at 0° C. The resulting mixture was concentrated under reduced pressure. The residue was purified by reversed-phase flash chromatography to afford 6′-fluorospiro[cyclopropane-1,3′-indoline] (290 mg, 63%) as a colorless oil. LC-MS (ES, m/z)=164.25 [M+1]⁺.

Step 3. Preparation of 3-(5-(6′-fluorospiro[cyclopropane-1,3′-indoline]-1′-carbonyl)-1-oxoisoindolin-2-yl)piperidine-2,6-dione. To a stirred solution of 6′-fluorospiro[cyclopropane-1,3′-indoline] (200 mg, 1.226 mmol, 1 equiv), 2-(2,6-dioxopiperidin-3-yl)-1-oxo-3H-isoindole-5-carboxylic acid (423.9 mg, 1.471 mmol, 1.2 equiv) and DIEA (475.2 mg, 3.678 mmol, 3 equiv) in DMF (3 mL) was added HATU (699.0 mg, 1.839 mmol, 1.5 equiv) in portions at room temperature under nitrogen atmosphere. The resulting mixture was stirred for overnight at room temperature under nitrogen atmosphere. The resulting mixture was diluted with water (100 mL). The resulting mixture was extracted with EtOAc (3×150 mL). The combined organic layers were washed with brine (2×150 mL), dried over anhydrous Na₂SO₄. After filtration, the filtrate was concentrated under reduced pressure. The crude product (80 mg) was purified by Prep-HPLC to afford 3-(5-(6′-fluorospiro[cyclopropane-1,3′-indoline]-1′-carbonyl)-1-oxoisoindolin-2-yl)piperidine-2,6-dione (21 mg, 4%) as an off-white solid. LC-MS (ES, m/z)=434.0 [M+1]⁺. ¹H NMR (400 MHz, DMSO-d₆) δ 11.01 (s, 1H), 7.97-7.77 (m, 3H), 7.74 (d, J=7.9 Hz, 1H), 6.87 (d, J=6.5 Hz, 2H), 5.15 (dd, J=13.3, 5.1 Hz, 1H), 4.53 (d, J=17.6 Hz, 1H), 4.40 (d, J=17.6 Hz, 1H), 4.04 (s, 2H), 2.93 (ddd, J=18.0, 13.5, 5.4 Hz, 1H), 2.72-2.54 (m, 1H), 2.41 (td, J=13.3, 4.6 Hz, 1H), 2.10-1.96 (m, 1H), 1.01 (d, J=17.4 Hz, 4H).

### Example 106

### Synthesis of 3-(5-(6-fluoroindoline-1-carbonyl)benzo[d]oxazol-2-yl)piperidine-2,6-dione

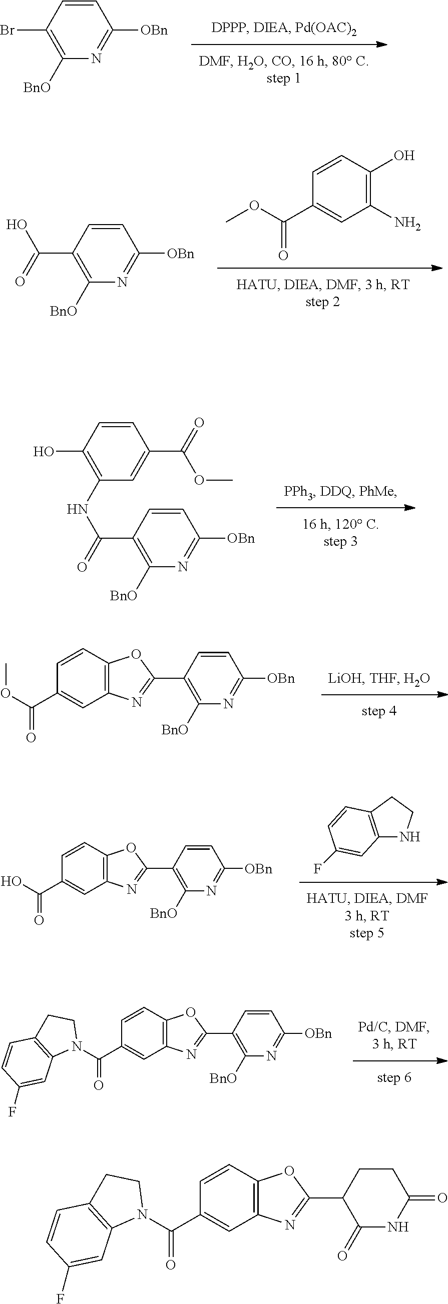

Step 1. Preparation of 2,6-bis(benzyloxy)pyridine-3-carboxylic acid. A solution of 2,6-bis(benzyloxy)-3-bromopyridine (4 g, 10.804 mmol, 1 equiv), DIEA (4.19 g, 32.412 mmol, 3 equiv) and Pd(OAc)₂ (485.1 mg, 2.161 mmol, 0.2 equiv) in DMF (50 mL)/H₂O (5 mL) was stirred for 2 min at room temperature. To the above mixture was added DPPP (445.6 mg, 1.080 mmol, 0.1 equiv) at room temperature. The resulting mixture was stirred for an additional 16 h at 80° C. under carbon monoxide atmosphere. The resulting mixture was filtered, filter cake was washed with THF (3×150 mL). The resulting mixture was diluted with water and extracted with CH₂Cl₂ (3×200 mL). The combined organic layers were washed with brine (3×200 mL), dried over anhydrous Na₂SO₄. After filtration, the filtrate was concentrated under reduced pressure. The residue was purified by reversed-phase flash chromatography to afford 2,6-bis(benzyloxy)pyridine-3-carboxylic acid (1.1 g, 30%) as an off-white solid. LC-MS (ES, m/z)=336.25 [M+1]⁺.

Step 2. Preparation of methyl 3-(2,6-bis(benzyloxy)nicotinamido)-4-hydroxybenzoate. A solution of 2,6-bis(benzyloxy)pyridine-3-carboxylic acid (270 mg, 0.805 mmol, 1 equiv), DIEA (312.2 mg, 2.415 mmol, 3 equiv) and orthoform (269.2 mg, 1.610 mmol, 2 equiv) in DMF (3 mL) was stirred for 2 min at room temperature. To the above mixture was added HATU (459.2 mg, 1.208 mmol, 1.5 equiv) at room temperature. The resulting mixture was stirred for an additional 3 h at room temperature. The resulting mixture was diluted with water and extracted with EtOAc (3×50 mL). The combined organic layers were washed with brine (3×50 mL), dried over anhydrous Na₂SO₄. After filtration, the filtrate was concentrated under reduced pressure. The residue was purified by reversed-phase flash chromatography to afford methyl 3-(2,6-bis(benzyloxy)nicotinamido)-4-hydroxybenzoate (200 mg, 51%) as an off-white solid. LC-MS (ES, m/z)=485.25 [M+1]⁺.

Step 3. Preparation of methyl 2-(2,6-bis(benzyloxy)pyridin-3-yl)benzo[d]oxazole-5-carboxylate. A solution of methyl 3-[2,6-bis(benzyloxy)pyridine-3-amido]-4-hydroxybenzoate (200 mg, 0.413 mmol, 1 equiv) in PhMe (2 mL) was treated with PPh₃ (162.4 mg, 0.619 mmol, 1.5 equiv) for 5 min at room temperature under nitrogen atmosphere followed by the addition of DDQ (140.6 mg, 0.619 mmol, 1.5 equiv) in portions at room temperature. The resulting mixture was stirred for an additional 16 h at 120° C. The resulting mixture was concentrated under vacuum. The resulting mixture was diluted with water and extracted with EtOAc (3×30 mL). The combined organic layers were washed with brine (3×50 mL), dried over anhydrous Na₂SO₄. The residue was purified by reversed-phase flash chromatography to afford methyl 2-(2,6-bis(benzyloxy)pyridin-3-yl)benzo[d]oxazole-5-carboxylate (150 mg, 78%) as an off-white solid. LC-MS (ES, m/z)=467.30 [M+1]⁺.

Step 4. Preparation of 2-(2,6-bis(benzyloxy)pyridin-3-yl)benzo[d]oxazole-5-carboxylic acid. A solution of methyl 2-(2,6-bis(benzyloxy)pyridin-3-yl)benzo[d]oxazole-5-carboxylate (200 mg, 0.429 mmol, 1 equiv) and LiOH (102.7 mg, 4.290 mmol, 10 equiv) in THF (3 mL) and H₂O (1 mL) was stirred for 3 h at room temperature. The resulting mixture was diluted with water and extracted with EtOAc (3×30 mL), the water phase acidified to pH 3 with 1 N HCl. The residue was extracted with EtOAc (3×30 mL). The combined organic layers were washed with brine (3×20 mL), dried over anhydrous Na₂SO₄. After filtration, the filtrate was concentrated under reduced pressure to afford 2-(2,6-bis(benzyloxy)pyridin-3-yl)benzo[d]oxazole-5-carboxylic acid (160 mg, 82%) as an off-white solid. LC-MS (ES, m/z)=453.25 [M+1]⁺.

Step 5. Preparation of (2-(2,6-bis(benzyloxy)pyridin-3-yl)benzo[d]oxazol-5-yl)(6-fluoroindolin-1-yl)methanone. To a solution of 2-(2,6-bis(benzyloxy)pyridin-3-yl)benzo[d]oxazole-5-carboxylic acid (110 mg, 0.243 mmol, 1 equiv), DIEA (94.3 mg, 0.729 mmol, 3 equiv) and 6-fluoro-2,3-dihydro-1H-indole (133.4 mg, 0.972 mmol, 4 equiv) in DMF (3 mL) was added HATU (138.7 mg, 0.364 mmol, 1.5 equiv) at room temperature. The resulting mixture was stirred for an additional 3 h at room temperature. The resulting mixture was diluted with water and extracted with EtOAc (3×30 mL). The combined organic layers were washed with brine (3×50 mL), dried over anhydrous Na₂SO₄. The residue was purified by reversed-phase flash chromatography to afford (2-(2,6-bis(benzyloxy)pyridin-3-yl)benzo[d]oxazol-5-yl)(6-fluoroindolin-1-yl)methanone (100 mg, 72%) as an off-white solid. LC-MS (ES, m/z)=572.40 [M+1]⁺.

Step 6. Preparation of 3-(5-(6-fluoroindoline-1-carbonyl)benzo[d]oxazol-2-yl)piperidine-2,6-dione. A solution of (2-(2,6-bis(benzyloxy)pyridin-3-yl)benzo[d]oxazol-5-yl)(6-fluoroindolin-1-yl)methanone (90 mg, 0.157 mmol, 1 equiv) and Pd/C (83.9 mg, 10% on carbon (wetted with ca.55% water)) in DMF (1.5 mL) was stirred for 3 h at room temperature under hydrogen atmosphere. The resulting mixture was filtered, the filter cake was washed with THF (3×10 mL). The filtrate was concentrated under reduced pressure. The residue was purified by reversed-phase flash chromatography to afford 3-(5-(6-fluoroindoline-1-carbonyl)benzo[d]oxazol-2-yl)piperidine-2,6-dione (12 mg, 19%) as an off-white solid. LC-MS (ES, m/z)=394.15 [M+1]⁺. ¹H NMR (400 MHz, DMSO-d₆) δ 11.18 (s, 1H), 8.05 (d, J=1.6 Hz, 1H), 7.88 (d, J=8.4 Hz, 1H), 7.68 (d, J=1.7 Hz, 1H), 7.66 (d, J=1.7 Hz, 1H), 7.33-7.24 (m, 1H), 6.89 (td, J=8.8, 2.6 Hz, 1H), 4.58 (dd, J=11.2, 5.1 Hz, 1H), 4.09 (t, J=8.4 Hz, 2H), 3.06 (t, J=8.3 Hz, 2H), 2.70 (qt, J=17.2, 5.1 Hz, 2H), 2.36 (ddd, J=13.2, 8.7, 4.8 Hz, 2H).

### Example 107

### Synthesis of 3-(6-(6-fluoroindoline-1-carbonyl)benzo[d]oxazol-2-yl)piperidine-2,6-dione

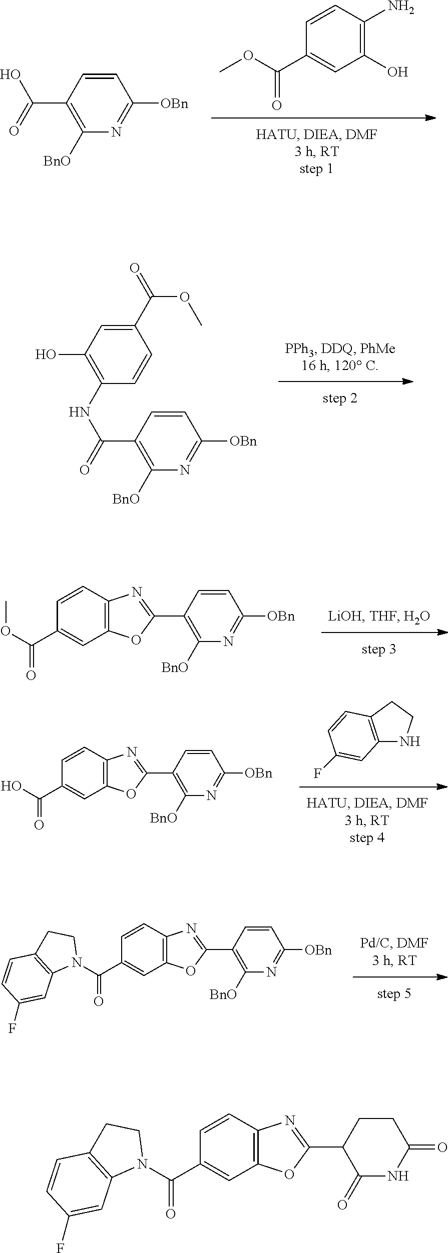

Step 1. Preparation of methyl 4-(2,6-bis(benzyloxy)nicotinamido)-3-hydroxybenzoate. To a solution of 2,6-bis(benzyloxy)pyridine-3-carboxylic acid (500 mg, 1.491 mmol, 1 equiv), DIEA (578.1 mg, 4.473 mmol, 3 equiv) and methyl 4-amino-3-hydroxybenzoate (498.7 mg, 2.982 mmol, 2 equiv) in DMF (3 mL) was added HATU (850.4 mg, 2.237 mmol, 1.5 equiv) at room temperature. The resulting mixture was stirred for an additional 3 h at room temperature. The resulting mixture was diluted with water (50 mL) and extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford methyl 4-(2,6-bis(benzyloxy)nicotinamido)-3-hydroxybenzoate (380 mg, 53%) as an off-white solid. LC-MS (ES, m/z)=485.30 [M+1]⁺.

Step 2. Preparation of methyl 2-(2,6-bis(benzyloxy)pyridin-3-yl)benzo[d]oxazole-6-carboxylate. A solution of methyl 4-(2,6-bis(benzyloxy)nicotinamido)-3-hydroxybenzoate (330 mg, 0.681 mmol, 1 equiv), PPh₃ (268.0 mg, 1.022 mmol, 1.5 equiv) and DDQ (232.0 mg, 1.022 mmol, 1.5 equiv) in PhMe (4 mL) was stirred for 16 h at 120° C. The resulting mixture was concentrated under vacuum. The residue was purified by reversed-phase flash chromatography to afford methyl 2-(2,6-bis(benzyloxy)pyridin-3-yl)benzo[d]oxazole-6-carboxylate (150 mg, 47%) as an off-white solid. LC-MS (ES, m/z)=467.35 [M+1]⁺.

Step 3. Preparation of 2-(2,6-bis(benzyloxy)pyridin-3-yl)benzo[d]oxazole-6-carboxylic acid. A solution of methyl 2-(2,6-bis(benzyloxy)pyridin-3-yl)benzo[d]oxazole-6-carboxylate (170 mg, 0.364 mmol, 1 equiv) and LiOH (174.7 mg, 7.280 mmol, 20 equiv) in THF (3 mL)/H₂O (1 mL) was stirred for 3 h at room temperature. The resulting mixture was concentrated under reduced pressure and diluted with water. The residue was acidified to pH 3 with 1 N HCl. The resulting mixture was extracted with EtOAc (3×30 mL). The combined organic layers were washed with brine (3×50 mL), dried over anhydrous Na₂SO₄. After filtration, the filtrate was concentrated under reduced pressure to afford 2-(2,6-bis(benzyloxy)pyridin-3-yl)benzo[d]oxazole-6-carboxylic acid (150 mg, 91%) as an off-white solid. LC-MS (ES, m/z)=453.10 [M+1]⁺.

Step 4. Preparation of (2-(2,6-bis(benzyloxy)pyridin-3-yl)benzo[d]oxazol-6-yl)(6-fluoroindolin-1-yl)methanone. To a solution of 2-(2,6-bis(benzyloxy)pyridin-3-yl)benzo[d]oxazole-6-carboxylic acid (110 mg, 0.243 mmol, 1 equiv), DIEA (94.3 mg, 0.729 mmol, 3 equiv) and 6-fluoro-2,3-dihydro-1H-indole (133.4 mg, 0.972 mmol, 4 equiv) in DMF (3 mL) was added HATU (138.7 mg, 0.364 mmol, 1.5 equiv) at room temperature. The resulting mixture was stirred for an additional 3 h at room temperature. The resulting mixture was diluted with water (30 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(6-fluoroindolin-1-yl)methanone (105 mg, 76%) as an off-white solid. LC-MS (ES, m/z)=572.40 [M+1]⁺.

Step 5. Preparation of 3-(6-(6-fluoroindoline-1-carbonyl)benzo[d]oxazol-2-yl)piperidine-2,6-dione. A solution of (2-(2,6-bis(benzyloxy)pyridin-3-yl)benzo[d]oxazol-6-yl)(6-fluoroindolin-1-yl)methanone (105 mg, 0.184 mmol, 1 equiv) and Pd/C (97.7 mg, 10% on carbon (wetted with ca. 55% water)) in DMF (2 mL) was stirred for 3 h at room temperature under hydrogen atmosphere. The resulting mixture was filtered, the filter cake was washed with THF (3×10 mL). The filtrate was concentrated under reduced pressure. The residue was purified by reversed-phase flash chromatography with the following conditions: column, C18 silica gel; mobile phase, MeCN in Water (0.1% FA), 0% to 90% gradient in 10 min; detector, UV 254 nm. The resulting mixture was concentrated under vacuum. The crude product (60 mg) was purified by Prep-HPLC to afford 3-(6-(6-fluoroindoline-1-carbonyl)benzo[d]oxazol-2-yl)piperidine-2,6-dione (3.0 mg, 4.15%) as an off-white solid. LC-MS (ES, m/z)=394.25 [M+1]⁺. ¹H NMR (300 MHz, DMSO-d₆) δ 11.18 (s, 1H), 8.08 (d, J=1.5 Hz, 1H), 7.91-7.61 (m, 3H), 7.30 (dd, J=8.2, 5.8 Hz, 1H), 6.95-6.81 (m, 1H), 4.58 (dd, J=11.2, 5.1 Hz, 1H), 4.10 (t, J=8.3 Hz, 2H), 3.06 (t, J=8.2 Hz, 2H), 2.71 (dq, J=16.4, 5.4, 4.6 Hz, 2H), 2.43-2.26 (m, 2H).

### Example 108

### Synthesis of 3-(5-(6-fluoro-3,3-dimethylindoline-1-carbonyl)-1-oxoisoindolin-2-yl)piperidine-2,6-dione

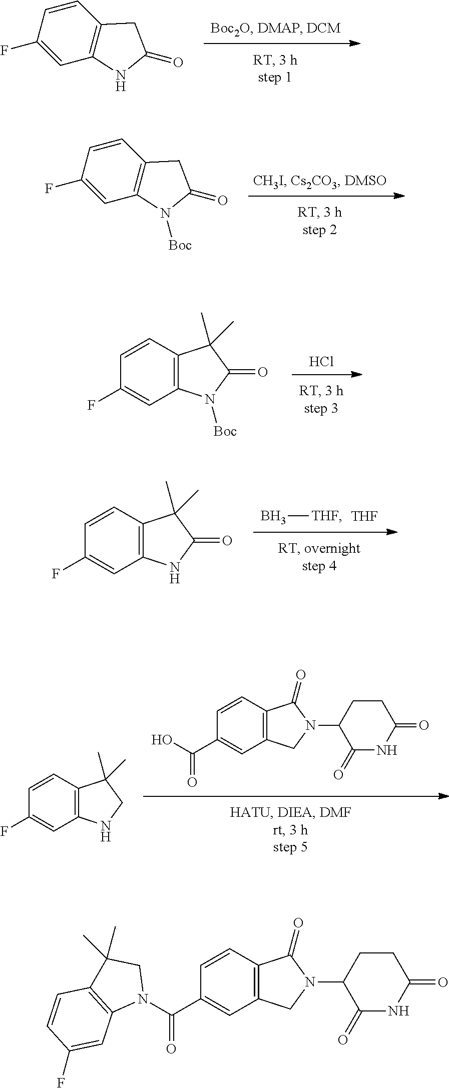

Step 1. Preparation of tert-butyl 6-fluoro-2-oxoindoline-1-carboxylate. To a stirred mixture of 6-fluoro-1,3-dihydroindol-2-one (1 g, 6.616 mmol, 1 equiv) in DCM (10 mL) were added Boc₂O (1.59 g, 7.278 mmol, 1.1 equiv) and DMAP (161.67 mg, 1.323 mmol, 0.2 equiv) at 0° C. The resulting mixture was stirred for 3 h at room temperature. The resulting mixture was diluted with water and extracted with DCM (3×100 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 6-fluoro-2-oxoindoline-1-carboxylate (1.5 g, 90%) as a yellow oil. LC-MS (ES, m/z)=250.15 [M−1]⁻.

Step 2. Preparation of tert-butyl 6-fluoro-3,3-dimethyl-2-oxoindoline-1-carboxylate. To a stirred mixture of tert-butyl 6-fluoro-2-oxoindoline-1-carboxylate (500 mg, 1.990 mmol, 1 equiv) and Cs₂CO₃ (2.59 g, 7.960 mmol, 4 equiv) in DMSO (10 mL) were added CH₃I (0.5 mL, 7.960 mmol, 4 equiv) at 0° C. The resulting mixture was stirred for 3 h at room temperature. The resulting mixture was diluted with water and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6-fluoro-3,3-dimethyl-2-oxoindoline-1-carboxylate (500 mg, 90%) as a white solid. LC-MS (ES, m/z)=280.40 [M+1]⁺.

Step 3. Preparation of 6-fluoro-3,3-dimethylindolin-2-one. To a stirred mixture of tert-butyl 6-fluoro-3,3-dimethyl-2-oxoindoline-1-carboxylate (500 mg, 1.790 mmol, 1 equiv) in dioxane (5 mL) were added HCl (gas) in 1,4-dioxane (5 mL, 4 mol/L in dioxane) dropwise at 0° C. The resulting mixture was stirred for 3 h at room temperature. The resulting mixture was concentrated under reduced pressure. The residue was purified by reversed-phase flash chromatography to afford 6-fluoro-3,3-dimethylindolin-2-one (300 mg, 94%) as a white solid. LC-MS (ES, m/z)=180.15 [M+1]⁺.

Step 4. Preparation of 6-fluoro-3,3-dimethylindoline. To a stirred mixture of 6-fluoro-3,3-dimethylindolin-2-one (500 mg, 2.790 mmol, 1 equiv) in THF (3 mL) was added BH₃-THF (14 mL, 14.000 mmol, 5.02 equiv) dropwise at 0° C. The resulting mixture was stirred overnight at room temperature. The reaction was quenched with Water at 0° C. and extracted with EtOAc (3×30 mL). The combined organic layers were washed with brine (2×20 mL), dried over anhydrous Na₂SO₄. After filtration, the filtrate was concentrated under reduced pressure. The residue was purified by reversed-phase flash chromatography to afford 6-fluoro-3,3-dimethylindoline (200 mg, 43%) as a yellow oil. LC-MS (ES, m/z)=166.25 [M+1]⁺.

Step 5. Preparation of 3-(5-(6-fluoro-3,3-dimethylindoline-1-carbonyl)-1-oxoisoindolin-2-yl)piperidine-2,6-dione. To a stirred mixture of 6-fluoro-3,3-dimethylindoline (115 mg, 0.696 mmol, 1 equiv) and 2-(2,6-dioxopiperidin-3-yl)-1-oxo-3H-isoindole-5-carboxylic acid (200.7 mg, 0.696 mmol, 1 equiv) in DMF (2 mL) were added HATU (397 mg, 1.044 mmol, 1.5 equiv) and DIEA (269.9 mg, 2.088 mmol, 3 equiv) at room temperature. The resulting mixture was stirred for 3 h at room temperature. The resulting mixture was diluted with water (20 mL) and extracted with EtOAc (3×30 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(5-(6-fluoro-3,3-dimethylindoline-1-carbonyl)-1-oxoisoindolin-2-yl)piperidine-2,6-dione (19 mg, 6%) as a white solid. LC-MS (ES, m/z)=436.15 [M+1]⁺. ¹H NMR (300 MHz, DMSO-d₆) δ 11.01 (s, 1H), 7.85 (d, J=7.9 Hz, 3H), 7.73 (d, J=7.9 Hz, 1H), 7.32 (dd, J=8.3, 5.8 Hz, 1H), 6.93 (s, 1H), 5.16 (dd, J=13.3, 5.0 Hz, 1H), 4.55 (d, J=17.7 Hz, 1H), 4.42 (d, J=17.6 Hz, 1H), 3.81 (s, 2H), 3.00-2.86 (m, 1H), 2.62 (d, J=16.6 Hz, 1H), 2.43 (dd, J=13.2, 4.5 Hz, 1H), 2.03 (s, 1H), 1.25 (s, 6H).

### Example 109

### Synthesis of 3-(5-(4,5-difluoroisoindoline-2-carbonyl)-1-oxoisoindolin-2-yl)piperidine-2,6-dione

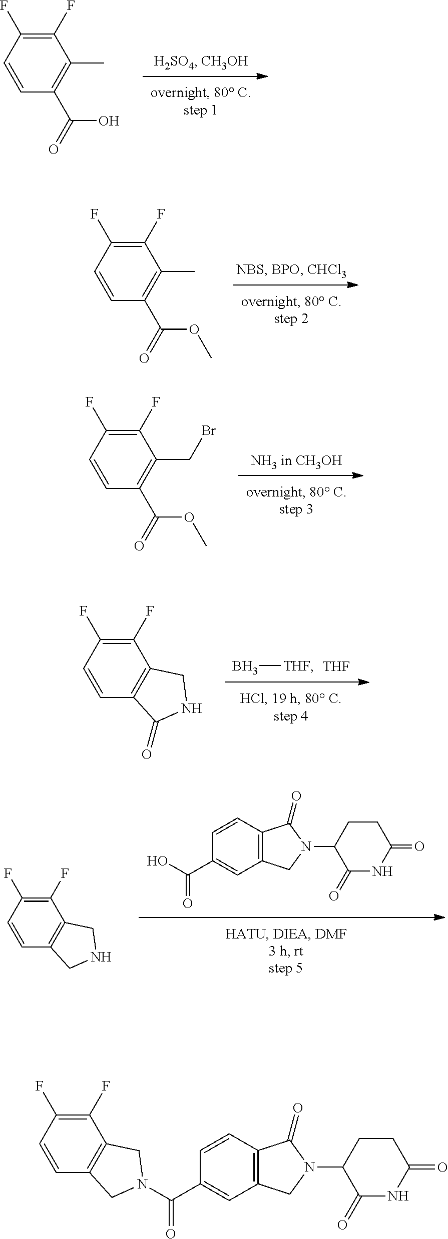

Step 1. Preparation of methyl 3,4-difluoro-2-methylbenzoate. To a stirred mixture of 3,4-difluoro-2-methylbenzoic acid (1 g, 5.810 mmol, 1 equiv) in CH₃OH (10 mL) was added H₂SO₄ (114.0 mg, 1.162 mmol, 0.2 equiv) at 0° C. The resulting mixture was stirred overnight at 80° C. under nitrogen atmosphere. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford methyl 3,4-difluoro-2-methylbenzoate (1 g, 92%) as a white oil. LC-MS (ES, m/z)=no mass signal.

Step 2. Preparation of methyl 2-(bromomethyl)-3,4-difluorobenzoate. To a stirred mixture of methyl 3,4-difluoro-2-methylbenzoate (1 g, 5.372 mmol, 1 equiv) in CHCl₃ (15 mL) was added NBS (1.43 g, 8.058 mmol, 1.5 equiv) and BPO (137.7 mg, 0.537 mmol, 0.1 equiv) at room temperature. The resulting mixture was stirred overnight at 80° C. under nitrogen atmosphere. The resulting mixture was diluted with water (100 mL) and extracted with EtOAc (3×100 mL). The combined organic layers were washed with brine (2×100 mL), dried over anhydrous Na₂SO₄. After filtration, the filtrate was concentrated under reduced pressure. The residue was purified by reversed-phase flash chromatography to afford methyl 2-(bromomethyl)-3,4-difluorobenzoate (1.1 g, 77%) as a white oil. LC-MS (ES, m/z)=no mass signal.

Step 3. Preparation of 4,5-difluoroisoindolin-1-one. To a stirred mixture of methyl 2-(bromomethyl)-3,4-difluorobenzoate (1 g, 3.773 mmol, 1 equiv) in CH₃OH (10 mL) was added NH₃ (7M) in MeOH (2.1 mL, 14.700 mmol, 3.90 equiv) at room temperature. The resulting mixture was stirred overnight at 80° C. The resulting mixture was concentrated under reduced pressure. The residue was purified by reversed-phase flash chromatography to afford 4,5-difluoroisoindolin-1-one (500 mg, 78%) as a white solid. LC-MS (ES, m/z)=168.10 [M−1].

Step 4. Preparation of 4,5-difluoroisoindoline. To a stirred mixture of 4,5-difluoroisoindolin-1-one (300 mg, 1.774 mmol, 1 equiv) in THF (6 mL) was added BH₃-THF (9 mL, 9.000 mmol, 5.07 equiv) at 0° C. The resulting mixture was stirred for 3 h at 80° C. The mixture was allowed to cool down to room temperature and quenched by the addition of MeOH (2 mL) at 0° C. The resulting mixture was concentrated under reduced pressure. To the above mixture was added HCl (6M) (3 mL, 18.000 mmol, 10.15 equiv) at 0° C. The resulting mixture was stirred for additional overnight at 100° C. The resulting mixture was concentrated under reduced pressure. The residue was purified by reversed-phase flash chromatography to afford 4,5-difluoroisoindoline (200 mg, 73%) as a white solid. LC-MS (ES, m/z)=156.20 [M+1]⁺.

Step 5. Preparation of 3-(5-(4,5-difluoroisoindoline-2-carbonyl)-1-oxoisoindolin-2-yl)piperidine-2,6-dione. To a stirred mixture of 4,5-difluoroisoindoline (150 mg, 0.967 mmol, 1 equiv) and 2-(2,6-dioxopiperidin-3-yl)-1-oxo-3H-isoindole-5-carboxylic acid (278.7 mg, 0.967 mmol, 1 equiv) in DMF (6 mL) were added HATU (551.43 mg, 1.450 mmol, 1.5 equiv) and DIEA (374.9 mg, 2.901 mmol, 3 equiv) at room temperature. The resulting mixture was stirred for 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(4,5-difluoroisoindoline-2-carbonyl)-1-oxoisoindolin-2-yl)piperidine-2,6-dione (32 mg, 8%) as a white solid. LC-MS (ES, m/z)=426.25 [M+1]⁺. ¹H NMR (300 MHz, DMSO-d₆) δ 11.01 (s, 1H), 7.87-7.80 (m, 2H), 7.76-7.69 (m, 1H), 7.47-7.33 (m, 1H), 7.27 (dd, J=8.3, 4.0 Hz, 1H), 7.11 (dd, J=8.4, 3.9 Hz, 1H), 5.15 (dd, J=13.3, 5.1 Hz, 1H), 4.94 (d, J=36.5 Hz, 2H), 4.77 (s, 1H), 4.54 (dd, J=17.5, 6.7 Hz, 1H), 4.41 (dd, J=17.6, 5.7 Hz, 1H), 2.93 (ddd, J=18.2, 13.5, 5.2 Hz, 1H), 2.61 (d, J=16.8 Hz, 1H), 2.47-2.36 (m, 1H), 2.07-2.00 (m, 1H).

### Example 110

### Synthesis of 3-(1-oxo-5-(spiro[cyclobutane-1,3′-indoline]-1′-carbonyl)isoindolin-2-yl)piperidine-2,6-dione

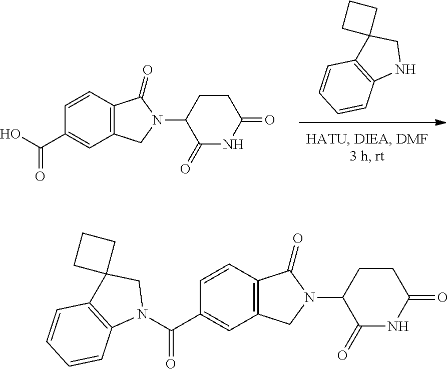 [SMILES: O=C1CCC(N2Cc3cc(C(=O)O)ccc3C2=O)C(=O)N1 ; c1ccc2c(c1)NCC21CCC1 ; O=C1CCC(N2Cc3cc(C(=O)N4CC5(CCC5)c5ccccc54)ccc3C2=O)C(=O)N1]

Step 1. Preparation of 3-(1-oxo-5-(spiro[cyclobutane-1,3′-indoline]-1′-carbonyl)isoindolin-2-yl)piperidine-2,6-dione. To a stirred mixture of 1′,2′-dihydrospiro[cyclobutane-1,3′-indole] (100 mg, 0.628 mmol, 1 equiv) and 2-(2,6-dioxopiperidin-3-yl)-1-oxo-3H-isoindole-5-carboxylic acid (181 mg, 0.628 mmol, 1 equiv) in DMF (2 mL) were added HATU (358.2 mg, 0.942 mmol, 1.5 equiv) and DIEA (243.5 mg, 1.884 mmol, 3 equiv) at room temperature. The resulting mixture was stirred for 3 h at room temperature. The resulting mixture was diluted with water and extracted with EtOAc (3×30 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(1-oxo-5-(spiro[cyclobutane-1,3′-indoline]-1′-carbonyl)isoindolin-2-yl)piperidine-2,6-dione (12.9 mg, 4.78%) as a white solid. LC-MS (ES, m/z)=430.2 [M+1]⁺. ¹H NMR (400 MHz, DMSO-d₆) δ 11.02 (s, 1H), 7.88-7.81 (m, 2H), 7.74 (s, 1H), 7.57 (d, J=7.4 Hz, 1H), 7.24 (s, 1H), 7.16 (s, 1H), 5.16 (dd, J=13.3, 5.1 Hz, 1H), 4.55 (d, J=17.6 Hz, 1H), 4.42 (d, J=17.5 Hz, 1H), 4.11 (s, 2H), 2.96-2.87 (m, 1H), 2.67-2.58 (m, 1H), 2.43 (s, 1H), 2.29 (s, 2H), 2.22 (s, 2H), 2.07 (s, 1H), 1.94 (s, 2H).

### Example 111

### Synthesis of 3-(5-(6-chloroindoline-1-carbonyl)-1-oxoisoindolin-2-yl)piperidine-2,6-dione

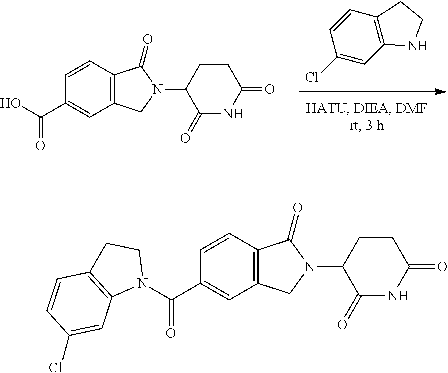 [SMILES: O=C1CCC(N2Cc3cc(C(=O)O)ccc3C2=O)C(=O)N1 ; Clc1ccc2c(c1)NCC2 ; O=C1CCC(N2Cc3cc(C(=O)N4CCc5ccc(Cl)cc54)ccc3C2=O)C(=O)N1]

Step 1. Preparation of 3-(5-(6-chloroindoline-1-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (188 mg, 0.652 mmol, 1 equiv) and 6-chloro-2,3-dihydro-1H-indole (100.2 mg, 0.652 mmol, 1 equiv) in DMF (2 mL) were added HATU (372 mg, 0.978 mmol, 1.5 equiv) and DIEA (252.9 mg, 1.956 mmol, 3 equiv) at room temperature. The resulting mixture was stirred for 3 h at room temperature. The resulting mixture was diluted with water and extracted with EtOAc (3×20 mL). The combined organic layers were washed with brine (2×30 mL), dried over anhydrous Na₂SO₄. After filtration, the filtrate was concentrated under reduced pressure. The crude product (80 mg) was purified by Prep-HPLC to afford 3-(5-(6-chloroindoline-1-carbonyl)-1-oxoisoindolin-2-yl)piperidine-2,6-dione (8 mg, 3%) as a white solid. LC-MS (ES, m/z)=424.05 [M+1]⁺. ¹H NMR (400 MHz, DMSO-d₆) δ 11.02 (s, 1H), 7.87-7.81 (m, 2H), 7.73 (d, J=8.0 Hz, 1H), 7.31 (d, J=8.0 Hz, 1H), 7.13 (d, J=7.8 Hz, 1H), 5.16 (dd, J=13.3, 5.2 Hz, 1H), 4.54 (d, J=17.7 Hz, 1H), 4.41 (d, J=17.6 Hz, 1H), 4.03 (t, J=8.3 Hz, 2H), 3.08 (t, J=8.3 Hz, 2H), 2.95-2.85 (m, 1H), 2.61 (d, J=17.1 Hz, 1H), 2.47-2.39 (m, 1H), 2.03 (d, J=12.4 Hz, 1H).

### Example 112

### Synthesis of 3-(5-(4-chloroisoindoline-2-carbonyl)-1-oxoisoindolin-2-yl)piperidine-2,6-dione

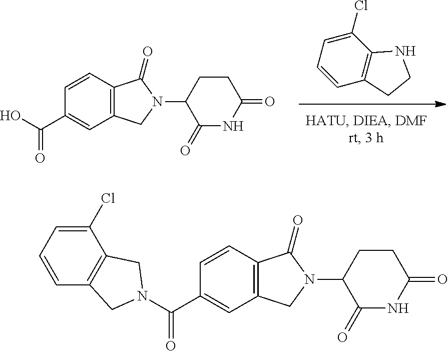 [SMILES: O=C1CCC(N2Cc3cc(C(=O)O)ccc3C2=O)C(=O)N1 ; Clc1cccc2c1NCC2 ; O=C1CCC(N2Cc3cc(C(=O)N4Cc5cccc(Cl)c5C4)ccc3C2=O)C(=O)N1]

Step 1. Preparation of 3-(5-(4-chloroisoindoline-2-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (188 mg, 0.652 mmol, 1 equiv) and 4-chloro-2,3-dihydro-1H-isoindole (100.2 mg, 0.652 mmol, 1 equiv) in DMF (2 mL) were added HATU (372 mg, 0.978 mmol, 1.5 equiv) and DIEA (252.9 mg, 1.956 mmol, 3 equiv) at room temperature. The resulting mixture was stirred for 3 h at room temperature. The resulting mixture was diluted with water and extracted with EtOAc (3×20 mL). The combined organic layers were washed with brine (2×30 mL), dried over anhydrous Na₂SO₄. After filtration, the filtrate was concentrated under reduced pressure. The residue was purified by reversed-phase flash chromatography to afford 3-(5-(4-chloroisoindoline-2-carbonyl)-1-oxoisoindolin-2-yl)piperidine-2,6-dione (18 mg, 7%) as a brown yellow solid. LC-MS (ES, m/z)=424.10 [M+1]⁺. ¹H NMR (400 MHz, DMSO-d₆) δ 11.02 (s, 1H), 7.88-7.80 (m, 2H), 7.74 (t, J=7.5 Hz, 1H), 7.43-7.30 (m, 3H), 5.16 (dd, J=13.3, 5.1 Hz, 1H), 4.87 (dd, J=49.8, 32.9 Hz, 4H), 4.59-4.39 (m, 2H), 2.99-2.86 (m, 1H), 2.62 (d, J 17.1 Hz, 1H), 2.43 (d, J=12.5 Hz, 1H), 2.05 (s, 1H).

### Example 113

### Synthesis of 3-(1-oxo-5-(2,6-diazaspiro[3.4]octane-6-carbonyl)isoindolin-2-yl)piperidine-2,6-dione

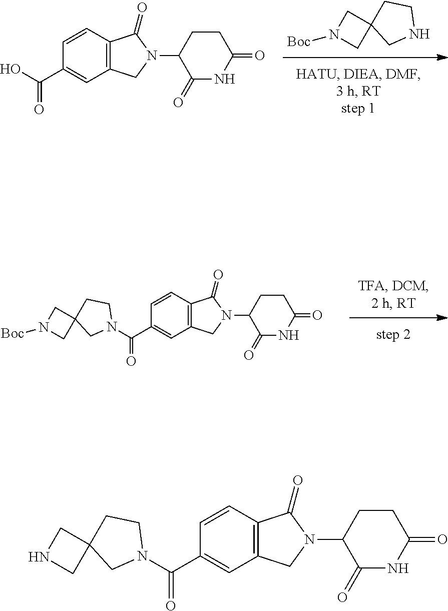 [SMILES: O=C1CCC(N2Cc3cc(C(=O)O)ccc3C2=O)C(=O)N1 ; CC(C)(C)OC(=O)N1CC2(CCNC2)C1 ; CC(C)(C)OC(=O)N1CC2(CCN(C(=O)c3ccc4c(c3)CN(C3CCC(=O)NC3=O)C4=O)C2)C1 ; O=C1CCC(N2Cc3cc(C(=O)N4CCC5(CNC5)C4)ccc3C2=O)C(=O)N1]

Step 1. Preparation of tert-butyl 6-(2-(2,6-dioxopiperidin-3-yl)-1-oxoisoindoline-5-carbonyl)-2,6-diazaspiro[3.4]octane-2-carboxylate. A solution of tert-butyl 2,6-diazaspiro[3.4]octane-2-carboxylate (150 mg, 0.707 mmol, 1 equiv), DIEA (273.9 mg, 2.121 mmol, 3 equiv) and 2-(2,6-dioxopiperidin-3-yl)-1-oxo-3H-isoindole-5-carboxylic acid (203.7 mg, 0.707 mmol, 1 equiv) in DMF (3 mL) was stirred for 3 min at room temperature. To the above mixture was added HATU (402.9 mg, 1.060 mmol, 1.5 equiv) at room temperature. The resulting mixture was stirred for an additional 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6-(2-(2,6-dioxopiperidin-3-yl)-1-oxoisoindoline-5-carbonyl)-2,6-diazaspiro[3.4]octane-2-carboxylate (120 mg, 35%) as an off-white solid. LC-MS (ES, m/z)=481.25 [M−1]⁻.

Step 2. Preparation of 3-(1-oxo-5-(2,6-diazaspiro[3.4]octane-6-carbonyl)isoindolin-2-yl)piperidine-2,6-dione. A solution of tert-butyl 6-(2-(2,6-dioxopiperidin-3-yl)-1-oxoisoindoline-5-carbonyl)-2,6-diazaspiro[3.4]octane-2-carboxylate (100 mg, 0.207 mmol, 1 equiv) in DCM (2 mL) was added TFA (0.4 mL) dropwise over 3 min at 0° C. The resulting mixture was stirred for an additional 2 h at room temperature. The resulting mixture was concentrated under vacuum. The residue was purified by reversed-phase flash chromatography to afford 35 mg crude. The crude product was purified by Prep-HPLC to afford 3-(1-oxo-5-(2,6-diazaspiro[3.4]octane-6-carbonyl)isoindolin-2-yl)piperidine-2,6-dione (6.0 mg, 8%) as an off-white solid. LC-MS (ES, m/z)=383.30 [M+1]⁺. ¹H NMR (400 MHz, DMSO-d₆) δ 8.38 (s, 1H), 7.83-7.68 (m, 2H), 7.62 (d, J=7.8 Hz, 1H), 5.14 (dt, J=13.2, 5.0 Hz, 1H), 4.62-4.28 (m, 3H), 3.88 (s, 2H), 3.58 (s, 1H), 3.52 (t, J=7.0 Hz, 3H), 3.40 (t, J=6.7 Hz, 1H), 2.92 (t, J=15.1 Hz, 1H), 2.61 (d, J=17.4 Hz, 1H), 2.40 (t, J=13.3 Hz, 1H), 2.19-1.94 (m, 3H).

### Example 114

### Synthesis of 3-(1-oxo-5-(2,7-diazaspiro[3.5]nonane-7-carbonyl)isoindolin-2-yl)piperidine-2,6-dione

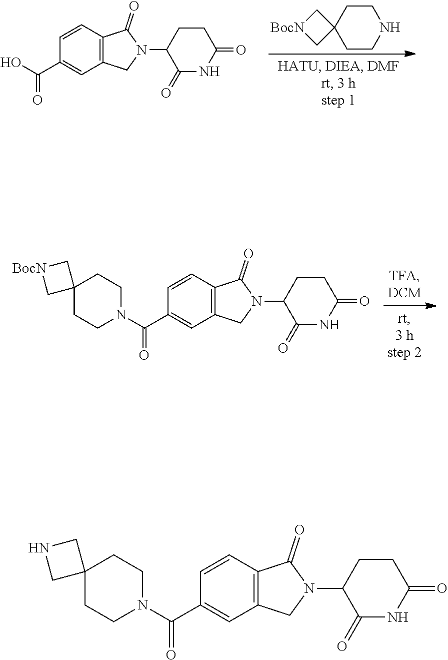 [SMILES: O=C1CCC(N2Cc3cc(C(=O)O)ccc3C2=O)C(=O)N1 ; CC(C)(C)OC(=O)N1CC2(CCNCC2)C1 ; CC(C)(C)OC(=O)N1CC2(CCN(C(=O)c3ccc4c(c3)CN(C3CCC(=O)NC3=O)C4=O)CC2)C1 ; O=C1CCC(N2Cc3cc(C(=O)N4CCC5(CC4)CNC5)ccc3C2=O)C(=O)N1]

Step 1. Preparation of tert-butyl 7-(2-(2,6-dioxopiperidin-3-yl)-1-oxoisoindoline-5-carbonyl)-2,7-diazaspiro[3.5]nonane-2-carboxylate. To a stirred mixture of tert-butyl 2,7-diazaspiro[3.5]nonane-2-carboxylate (339 mg, 1.498 mmol, 1.00 equiv) and 2-(2,6-dioxopiperidin-3-yl)-1-oxo-3H-isoindole-5-carboxylic acid (431.8 mg, 1.498 mmol, 1.00 equiv) in DMF (6 mL) was added DIEA (580.8 mg, 4.494 mmol, 3 equiv) and HATU (854.3 mg, 2.247 mmol, 1.5 equiv) at room temperature. The resulting mixture was stirred for 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 7-(2-(2,6-dioxopiperidin-3-yl)-1-oxoisoindoline-5-carbonyl)-2,7-diazaspiro[3.5]nonane-2-carboxylate (160 mg, 22%) as a white solid. LC-MS (ES, m/z)=495.30 [M−1]⁻.

Step 2. Preparation of 3-(1-oxo-5-(2,7-diazaspiro[3.5]nonane-7-carbonyl)isoindolin-2-yl)piperidine-2,6-dione. To a stirred mixture of tert-butyl 7-(2-(2,6-dioxopiperidin-3-yl)-1-oxoisoindoline-5-carbonyl)-2,7-diazaspiro[3.5]nonane-2-carboxylate (160 mg, 0.322 mmol, 1 equiv) in DCM (5 mL) was added TFA (1 mL) dropwise at room temperature. The resulting mixture was stirred for 3 h at room temperature. The resulting mixture was concentrated under reduced pressure. The crude product was purified by Prep-HPLC to afford 3-(1-oxo-5-(2,7-diazaspiro[3.5]nonane-7-carbonyl)isoindolin-2-yl)piperidine-2,6-dione (34 mg, 26%) as a white solid. LC-MS (ES, m/z)=397.20 [M+1]⁺. ¹H NMR (400 MHz, DMSO-d₆) δ 8.37 (s, 1H), 7.79 (d, J=7.8 Hz, 1H), 7.60 (s, 1H), 7.48 (d, J=7.7 Hz, 1H), 5.14 (dd, J=13.2, 5.1 Hz, 1H), 4.50 (d, J=17.6 Hz, 1H), 4.38 (d, J=17.5 Hz, 1H), 3.61 (d, J=27.6 Hz, 6H), 3.23 (s, 2H), 2.92 (ddd, J=18.1, 13.4, 5.4 Hz, 1H), 2.65-2.56 (m, 1H), 2.45-2.34 (m, 1H), 2.06-1.98 (m, 1H), 1.77 (d, J=36.4 Hz, 4H).

### Example 115

### Synthesis of 3-(5-(3-isopropylindoline-1-carbonyl)-1-oxoisoindolin-2-yl)piperidine-2,6-dione

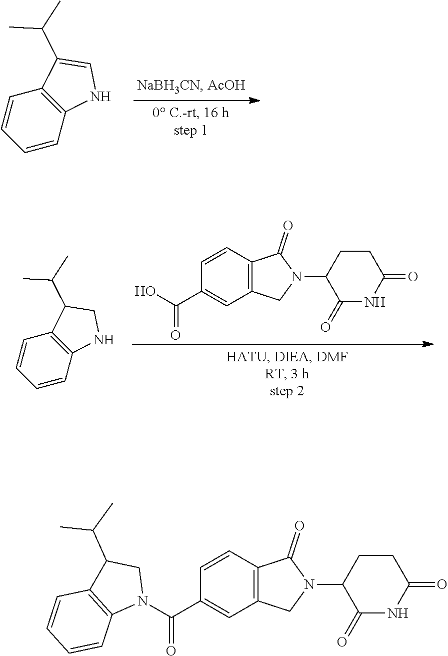 [SMILES: CC(C)c1c[nH]c2ccccc12 ; CC(C)C1CNc2ccccc21 ; O=C1CCC(N2Cc3cc(C(=O)O)ccc3C2=O)C(=O)N1 ; CC(C)C1CN(C(=O)c2ccc3c(c2)CN(C2CCC(=O)NC2=O)C3=O)c2ccccc21]

Step 1. Preparation of 3-isopropylindoline. To a stirred mixture of 3-isopropyl-1H-indole (1 g, 6.280 mmol, 1 equiv) in AcOH (10 mL, 174.515 mmol, 27.79 equiv) was added NaBH₃CN (1.58 g, 25.120 mmol, 4 equiv) at 0° C. The resulting mixture was stirred overnight at room temperature. The reaction was quenched by the addition of Water/Ice (10 mL) at 0° C. The resulting mixture was extracted with DCM (3×200 mL). The combined organic layers were washed with brine (2×100 mL), dried over anhydrous Na₂SO₄. After filtration, the filtrate was concentrated under reduced pressure. The residue was purified by reversed-phase flash chromatography to afford 3-isopropylindoline (500 mg, 49%) as yellow oil. LC-MS (ES, m/z)=162.25 [M+1]⁺.

Step 2. Preparation of 3-(5-(3-isopropylindoline-1-carbonyl)-1-oxoisoindolin-2-yl)piperidine-2,6-dione. To a stirred mixture of 3-isopropylindoline (100 mg, 0.620 mmol, 1 equiv) and 2-(2,6-dioxopiperidin-3-yl)-1-oxo-3H-isoindole-5-carboxylic acid (178.8 mg, 0.620 mmol, 1 equiv) in DMF (3.0 mL) were added HATU (353.7 mg, 0.930 mmol, 1.5 equiv) and DIEA (240.5 mg, 1.860 mmol, 3 equiv) at room temperature. The resulting mixture was stirred for 3 h at room temperature. The resulting mixture was diluted with water and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(3-isopropylindoline-1-carbonyl)-1-oxoisoindolin-2-yl)piperidine-2,6-dione (14 mg, 5%) as a white solid. LC-MS (ES, m/z)=432.15 [M+1]⁺. ¹H NMR (400 MHz, DMSO-d₆) δ 11.02 (s, 1H), 8.10 (s, 1H), 7.89-7.69 (m, 3H), 7.28 (t, J=14.0 Hz, 2H), 7.09 (s, 1H), 5.16 (dd, J=13.3, 5.1 Hz, 1H), 4.55 (dd, J=17.6, 4.0 Hz, 1H), 4.42 (dd, J=17.6, 5.3 Hz, 1H), 4.08 (ddd, J=11.1, 9.4, 5.7 Hz, 1H), 3.67 (s, 1H), 3.29 (d, J=4.7 Hz, 1H), 2.93 (ddd, J=18.2, 13.6, 5.3 Hz, 1H), 2.62 (d, J=17.1 Hz, 1H), 2.39 (dd, J=13.2, 4.4 Hz, 1H), 2.09-1.98 (m, 2H), 0.87-0.68 (m, 6H).

### Example 116

### Synthesis of 3-(5-(6′-fluorospiro[cyclobutane-1,3′-indoline]-1′-carbonyl)-1-oxoisoindolin-2-yl)piperidine-2,6-dione

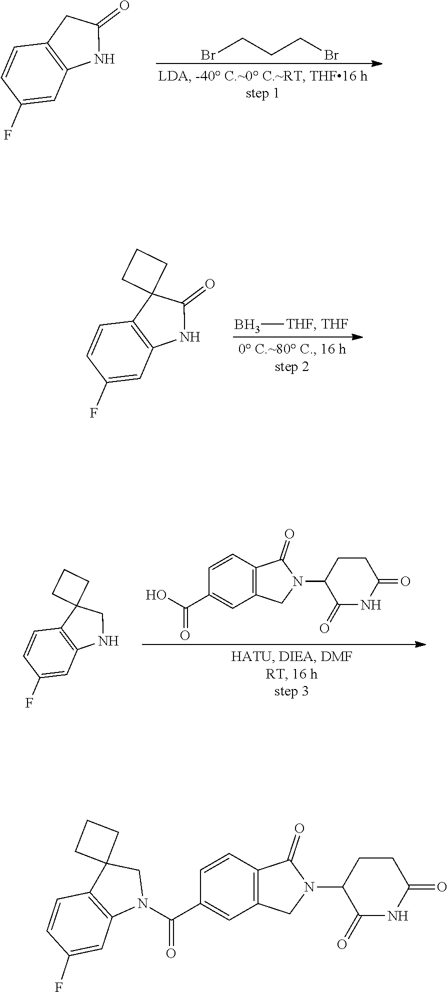

Step 1. Preparation of 6′-fluorospiro[cyclobutane-1,3′-indolin]-2′-one. To a stirred solution of 6-fluoro-1,3-dihydroindol-2-one (1 g, 6.616 mmol, 1 equiv) in THF (10 mL) was added LDA (in 2M THF) (2.8 g, 26.464 mmol, 4 equiv) dropwise at −40° C. under nitrogen atmosphere. The resulting mixture was stirred for 30 min at −40° C. under nitrogen atmosphere. To the above mixture was added 1,3-dibromopropane (4.0 g, 19.848 mmol, 3 equiv) dropwise over 5 min at 0° C. The resulting mixture was stirred for additional overnight at room temperature. The reaction was quenched by the addition of sat. NH₄Cl (aq.) (100 mL) at 0° C. The resulting mixture was diluted with water (200 mL). The resulting mixture was extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford 6′-fluorospiro[cyclobutane-1,3′-indolin]-2′-one (140 mg, 11%) as an off-white solid. LC-MS (ES, m/z)=192.15 [M+1]⁺.

Step 2. Preparation of 6′-fluorospiro[cyclobutane-1,3′-indoline]. To a stirred solution of 6′-fluorospiro[cyclobutane-1,3′-indolin]-2′-one (140 mg, 0.732 mmol, 1 equiv) in THF (2 mL) was added BH₃-THF (3.66 mL, 3.660 mmol, 5 equiv) dropwise at 0° C. under nitrogen atmosphere. The resulting mixture was stirred overnight at 80° C. under nitrogen atmosphere. The mixture was allowed to cool down to room temperature. The reaction was quenched with MeOH (20 mL) at 0° C. The resulting mixture was concentrated under reduced pressure. The residue was purified by reversed-phase flash chromatography to afford 6′-fluorospiro[cyclobutane-1,3′-indoline] (100 mg, 77%) as a colorless oil. LC-MS (ES, m/z)=178.10 [M+1]⁺.

Step 3. Preparation of 3-(5-(6′-fluorospiro[cyclobutane-1,3′-indoline]-1′-carbonyl)-1-oxoisoindolin-2-yl)piperidine-2,6-dione. To a stirred solution of 6′-fluorospiro[cyclobutane-1,3′-indoline] (100 mg, 0.564 mmol, 1 equiv), 2-(2,6-dioxopiperidin-3-yl)-1-oxo-3H-isoindole-5-carboxylic acid (325.3 mg, 1.128 mmol, 2 equiv) and DIEA (364.6 mg, 2.820 mmol, 5 equiv) in DMF (2 mL) was added HATU (321.8 mg, 0.846 mmol, 1.5 equiv) at room temperature under nitrogen atmosphere. The resulting mixture was stirred overnight at room temperature under nitrogen atmosphere. The resulting mixture was diluted with water (100 mL) and extracted with EtOAc (3×100 mL). The combined organic layers were washed with brine (2×150 mL), dried over anhydrous Na₂SO₄. After filtration, the filtrate was concentrated under reduced pressure. The crude product (70 mg) was purified by Prep-HPLC to afford 3-(5-(6′-fluorospiro[cyclobutane-1,3′-indoline]-1′-carbonyl)-1-oxoisoindolin-2-yl)piperidine-2,6-dione (13 mg, 5%) as an off-white solid. LC-MS (ES, m/z)=448.20 [M+1]⁺. ¹H NMR (400 MHz, DMSO-d₆) δ 11.03 (s, 1H), 7.80 (dd, J=43.0, 8.0 Hz, 4H), 7.58 (dd, J=8.3, 5.7 Hz, 1H), 6.99 (td, J=8.8, 2.5 Hz, 1H), 5.17 (dd, J=13.3, 5.1 Hz, 1H), 4.61-4.36 (m, 2H), 4.16 (s, 2H), 2.94 (ddd, J=18.1, 13.6, 5.4 Hz, 1H), 2.62 (dt, J=15.1, 2.9 Hz, 1H), 2.42 (td, J=13.2, 4.5 Hz, 1H), 2.30 (ddt, J=13.6, 9.0, 4.3 Hz, 2H), 2.25-2.15 (m, 2H), 2.12-1.99 (m, 1H), 1.92 (dq, J=15.5, 8.2, 6.5 Hz, 2H).

### Example 117

### Synthesis of 3-(1-oxo-5-(1,6-diazaspiro[3.4]octane-6-carbonyl)isoindolin-2-yl)piperidine-2,6-dione

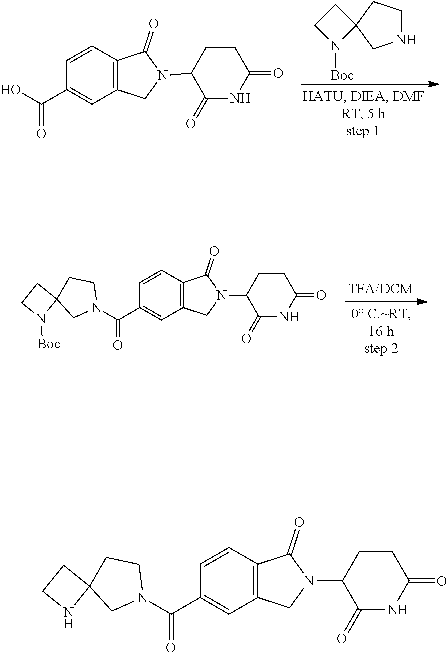 [SMILES: O=C1CCC(N2Cc3cc(C(=O)O)ccc3C2=O)C(=O)N1 ; CC(C)(C)OC(=O)N1CCC12CCNC2 ; CC(C)(C)OC(=O)N1CCC12CCN(C(=O)c1ccc3c(c1)CN(C1CCC(=O)NC1=O)C3=O)C2 ; O=C1CCC(N2Cc3cc(C(=O)N4CCC5(CCN5)C4)ccc3C2=O)C(=O)N1]

Step 1. Preparation of tert-butyl 6-(2-(2,6-dioxopiperidin-3-yl)-1-oxoisoindoline-5-carbonyl)-1,6-diazaspiro[3.4]octane-1-carboxylate. To a stirred solution of tert-butyl 1,6-diazaspiro[3.4]octane-1-carboxylate (100 mg, 0.471 mmol, 1 equiv), 2-(2,6-dioxopiperidin-3-yl)-1-oxo-3H-isoindole-5-carboxylic acid (162.9 mg, 0.565 mmol, 1.2 equiv) and DIEA (182.6 mg, 1.413 mmol, 3 equiv) in DMF (2 mL) was added HATU (268.7 mg, 0.706 mmol, 1.5 equiv) at room temperature under nitrogen atmosphere. The resulting mixture was stirred for 5 h at room temperature under nitrogen atmosphere. The resulting mixture was diluted with water (100 mL). The resulting mixture was extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford tert-butyl 6-(2-(2,6-dioxopiperidin-3-yl)-1-oxoisoindoline-5-carbonyl)-1,6-diazaspiro[3.4]octane-1-carboxylate (150 mg, 66%) as a colorless oil. LC-MS (ES, m/z)=481.30 [M+1]⁺.

Step 2. Preparation of 3-(1-oxo-5-(1,6-diazaspiro[3.4]octane-6-carbonyl)isoindolin-2-yl)piperidine-2,6-dione. To a stirred solution of tert-butyl 6-[2-(2,6-dioxopiperidin-3-yl)-1-oxo-3H-isoindole-5-carbonyl]-1,6-diazaspiro[3.4]octane-1-carboxylate (150 mg, 0.311 mmol, 1 equiv) in DCM (5 mL) was added TFA (1 mL) dropwise at 0° C. under nitrogen atmosphere. The resulting mixture was stirred for 16 h at room temperature under nitrogen atmosphere. The resulting mixture was concentrated under reduced pressure. The crude product (70 mg) was purified by Prep-HPLC to afford 3-(1-oxo-5-(1,6-diazaspiro[3.4]octane-6-carbonyl)isoindolin-2-yl)piperidine-2,6-dione (29.3 mg, 25%) as an off-white solid. LC-MS (ES, m/z)=383.25 [M+1]⁺. ¹H NMR (400 MHz, DMSO-d₆) δ 11.01 (s, 1H), 8.29 (s, 1H), 7.80 (d, J=7.8 Hz, 1H), 7.74 (d, J=9.2 Hz, 1H), 7.63 (t, J=8.5 Hz, 1H), 5.14 (dt, J=13.5, 4.7 Hz, 1H), 4.60-4.30 (m, 3H), 4.02-3.76 (m, 5H), 2.92 (ddt, J=20.6, 13.8, 4.4 Hz, 1H), 2.66-2.57 (m, 1H), 2.49-2.15 (m, 4H), 2.14-1.96 (m, 2H).

### Example 118

### Synthesis of 1-(7-(7-fluoroindoline-1-carbonyl)naphthalen-2-yl)dihydropyrimidine-2,4(1H,3H)-dione

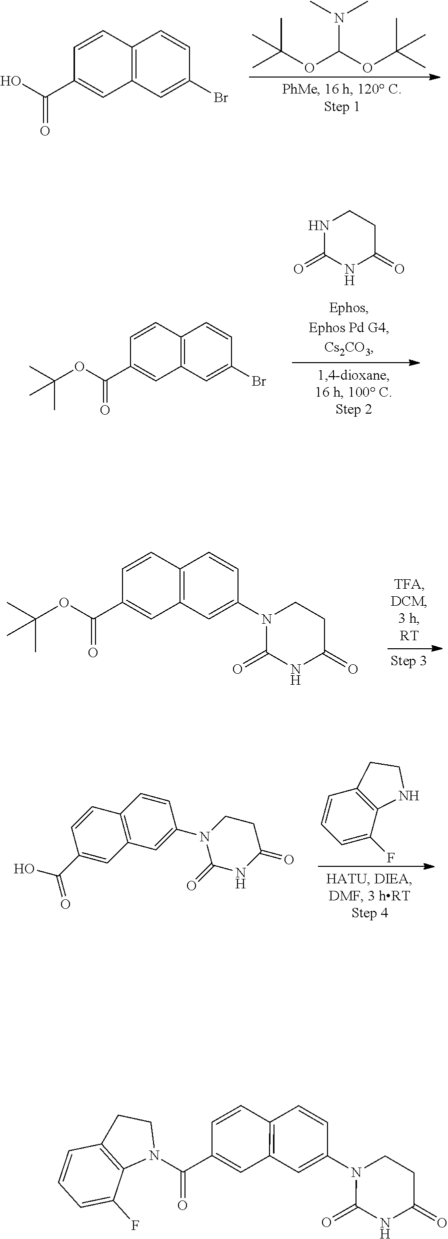

Step 1. Preparation of tert-butyl 7-bromo-2-naphthoate. To a stirred mixture of 7-bromonaphthalene-2-carboxylic acid (1.5 g, 5.974 mmol, 1 equiv) in methylbenzene (10 mL) was added [bis(tert-butoxy)methyl]dimethylamine (12.15 g, 59.740 mmol, 10 equiv) at room temperature. The resulting mixture was stirred for 16 h at 120° C. The resulting mixture was concentrated under reduced pressure. The residue diluted with water (200 mL) and extracted with EtOAc (3×100 mL). The combined organic layers were washed with brine (2×300 mL), dried over anhydrous Na₂SO₄. After filtration, the filtrate was concentrated under reduced pressure. The residue was purified by reversed-phase flash chromatography to afford tert-butyl 7-bromo-2-naphthoate (1.1 g, 60%) as a white solid. ¹H NMR (400 MHz, DMSO-d₆) δ 8.56 (d, J=1.7 Hz, 1H), 8.44 (d, J=1.9 Hz, 1H), 8.05 (d, J=8.6 Hz, 1H), 7.97 (td, J=5.3, 2.7 Hz, 2H), 7.77 (ddd, J=8.8, 2.1, 0.9 Hz, 1H), 1.60 (s, 9H).

Step 2. Preparation of tert-butyl 7-(2,4-dioxotetrahydropyrimidin-1(2H)-yl)-2-naphthoate. To a stirred mixture of tert-butyl 7-bromo-2-naphthoate (1 g, 3.255 mmol, 1 equiv), Cs₂CO₃ (2.12 g, 6.510 mmol, 2 equiv) and 5,6-dihydrouracil (1.11 g, 9.765 mmol, 3 equiv) in 1,4-dioxane (10 mL) were added Ephos (174.1 mg, 0.326 mmol, 0.1 equiv) and Ephos Pd G4 (299.0 mg, 0.326 mmol, 0.1 equiv) at room temperature. The resulting mixture was stirred for 16 h at 100° C. under nitrogen atmosphere. The resulting mixture was diluted with water (100 mL) and extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford tert-butyl 7-(2,4-dioxotetrahydropyrimidin-1(2H)-yl)-2-naphthoate (860 mg, 78%) as a white solid. LC-MS (ES, m/z)=339.10 [M−1]⁻.

Step 3. Preparation of 7-(2,4-dioxotetrahydropyrimidin-1(2H)-yl)-2-naphthoic acid. To a stirred mixture of tert-butyl 7-(2,4-dioxotetrahydropyrimidin-1(2H)-yl)-2-naphthoate (800 mg, 2.350 mmol, 1 equiv) in DCM (10 mL) was added TFA (2 mL) dropwise at 0° C. The resulting mixture was stirred for 3 h at room temperature. The resulting mixture was concentrated under reduced pressure. The residue was purified by reversed-phase flash chromatography to afford 7-(2,4-dioxotetrahydropyrimidin-1(2H)-yl)-2-naphthoic acid (600 mg, 90%) as a white solid. LC-MS (ES, m/z)=285.10 [M+1]⁺.

Step 4. Preparation of 1-(7-(7-fluoroindoline-1-carbonyl)naphthalen-2-yl)dihydropyrimidine-2,4(1H,3H)-dione. To a solution of 7-fluoro-2,3-dihydro-1H-indole (115.8 mg, 0.843 mmol, 3 equiv), DIEA (109.1 mg, 0.843 mmol, 3 equiv) and 7-(2,4-dioxotetrahydropyrimidin-1(2H)-yl)-2-naphthoic acid (80 mg, 0.281 mmol, 1.00 equiv) in DMF (3.0 mL) was added HATU (160.5 mg, 0.422 mmol, 1.5 equiv) at room temperature. The resulting mixture was stirred for an additional 3 h at room temperature. The resulting mixture was diluted with water (50 mL) and extracted with EtOAc (3×20 mL). The combined organic layers were washed with brine (2×50 mL), dried over anhydrous Na₂SO₄. After filtration, the filtrate was concentrated under reduced pressure. The crude product (70 mg) was purified by Prep-HPLC to afford 1-(7-(7-fluoroindoline-1-carbonyl)naphthalen-2-yl)dihydropyrimidine-2,4(1H,3H)-dione (10 mg, 9%) as an off-white solid. LC-MS (ES, m/z)=404.10 [M+1]⁺. ¹H NMR (400 MHz, DMSO-d₆) δ 10.48 (s, 1H), 8.28 (d, J=1.7 Hz, 1H), 8.01 (dd, J=8.7, 5.6 Hz, 2H), 7.96 (d, J=2.1 Hz, 1H), 7.74 (dd, J=8.5, 1.7 Hz, 1H), 7.67 (dd, J=8.8, 2.2 Hz, 1H), 7.19-7.09 (m, 2H), 7.04 (ddd, J=9.6, 7.9, 1.6 Hz, 1H), 4.19 (t, J=7.8 Hz, 2H), 3.94 (t, J=6.6 Hz, 2H), 3.13 (t, J=7.7 Hz, 2H), 2.77 (t, J=6.6 Hz, 2H).

### Example 119

### Synthesis of 1-(7-(5-fluoroindoline-1-carbonyl)naphthalen-2-yl)dihydropyrimidine-2,4(1H,3H)-dione

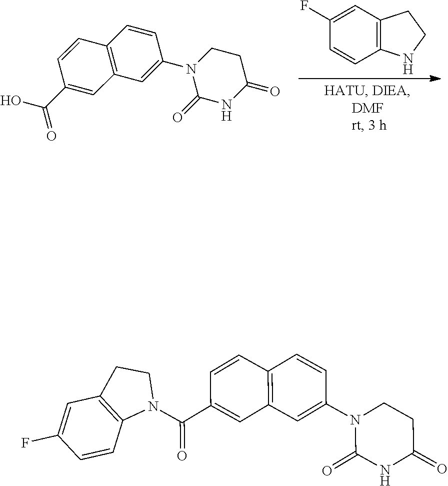 [SMILES: O=C1CCN(c2ccc3ccc(C(=O)O)cc3c2)C(=O)N1 ; Fc1ccc2c(c1)CCN2 ; O=C1CCN(c2ccc3ccc(C(=O)N4CCc5cc(F)ccc54)cc3c2)C(=O)N1]

Step 1. Preparation of 1-(7-(5-fluoroindoline-1-carbonyl)naphthalen-2-yl)dihydropyrimidine-2,4(1H,3H)-dione. To a stirred mixture of 7-(2,4-dioxotetrahydropyrimidin-1(2H)-yl)-2-naphthoic acid (100.0 mg, 0.352 mmol, 1 equiv) and 5-fluoro-2,3-dihydro-1H-indole (48.3 mg, 0.352 mmol, 1 equiv) in DMF (2 mL) were added HATU (200.7 mg, 0.528 mmol, 1.5 equiv) and DIEA (136.4 mg, 1.056 mmol, 3 equiv) at room temperature. The resulting mixture was stirred for 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 1-(7-(5-fluoroindoline-1-carbonyl)naphthalen-2-yl)dihydropyrimidine-2,4(1H,3H)-dione (39.9 mg, 28%) as a white solid. LC-MS (ES, m/z)=404.1 [M+1]⁺. ¹H NMR (400 MHz, DMSO-d₆) δ 10.47 (d, J=2.7 Hz, 1H), 8.16-7.92 (m, J=9.5 Hz, 5H), 7.66 (ddd, J=8.8, 3.8, 2.1 Hz, 2H), 7.17 (dd, J=8.6, 2.8 Hz, 1H), 7.02 (s, 1H), 4.11 (t, J=8.3 Hz, 2H), 3.94 (t, J=6.6 Hz, 2H), 3.11 (t, J=8.3 Hz, 2H), 2.78 (t, J=6.6 Hz, 2H).

### Example 120

### Synthesis of 1-(7-(indoline-1-carbonyl)naphthalen-2-yl)dihydropyrimidine-2,4(1H,3H)-dione

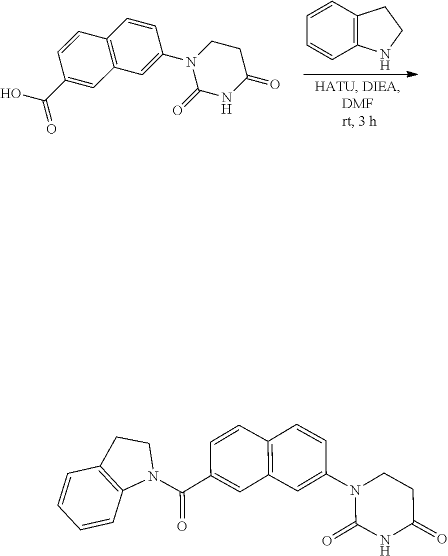 [SMILES: O=C1CCN(c2ccc3ccc(C(=O)O)cc3c2)C(=O)N1 ; c1ccc2c(c1)CCN2 ; O=C1CCN(c2ccc3ccc(C(=O)N4CCc5ccccc54)cc3c2)C(=O)N1]

Step 1. Preparation of 1-(7-(indoline-1-carbonyl)naphthalen-2-yl)dihydropyrimidine-2,4(1H,3H)-dione. A solution of indoline (100.6 mg, 0.843 mmol, 3 equiv), DIEA (109.1 mg, 0.843 mmol, 3 equiv) and 7-(2,4-dioxo-1,3-diazinan-1-yl)naphthalene-2-carboxylic acid (80.0 mg, 0.281 mmol, 1 equiv) in DMF (3 mL) was stirred for 3 min at room temperature. To the above mixture was added HATU (160.5 mg, 0.422 mmol, 1.5 equiv) at room temperature. The resulting mixture was stirred for an additional 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 1-(7-(indoline-1-carbonyl)naphthalen-2-yl)dihydropyrimidine-2,4(1H,3H)-dione (20 mg, 18%) as a light yellow solid. LC-MS (ES, m/z)=386.2 [M+1]⁺. ¹H NMR (400 MHz, DMSO-d₆) δ 10.48 (s, 1H), 8.27-7.96 (m, 4H), 7.93 (d, J=2.2 Hz, 1H), 7.70-7.61 (m, 2H), 7.29 (d, J=7.4 Hz, 1H), 7.17 (s, 1H), 7.05 (t, J=7.7 Hz, 1H), 4.08 (t, J=8.2 Hz, 2H), 3.94 (t, J=6.6 Hz, 2H), 3.10 (t, J=8.3 Hz, 2H), 2.77 (t, J=6.7 Hz, 2H).

### Example 121

### Synthesis of 3-(1-oxo-5-(2,6-diazaspiro[3.3]heptane-2-carbonyl)isoindolin-2-yl)piperidine-2,6-dione

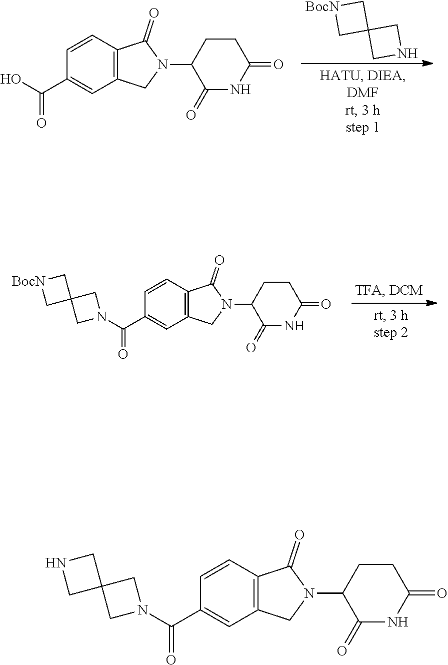 [SMILES: O=C1CCC(N2Cc3cc(C(=O)O)ccc3C2=O)C(=O)N1 ; CC(C)(C)OC(=O)N1CC2(CNC2)C1 ; CC(C)(C)OC(=O)N1CC2(C1)CN(C(=O)c1ccc3c(c1)CN(C1CCC(=O)NC1=O)C3=O)C2 ; O=C1CCC(N2Cc3cc(C(=O)N4CC5(CNC5)C4)ccc3C2=O)C(=O)N1]

Step 1. Preparation of tert-butyl 6-(2-(2,6-dioxopiperidin-3-yl)-1-oxoisoindoline-5-carbonyl)-2,6-diazaspiro[3.3]heptane-2-carboxylate. To a stirred mixture of 2-(2,6-dioxopiperidin-3-yl)-1-oxo-3H-isoindole-5-carboxylic acid (145.0 mg, 0.503 mmol, 1 equiv) and tert-butyl 2,6-diazaspiro[3.3]heptane-2-carboxylate (99.7 mg, 0.503 mmol, 1 equiv) in DMF (2 mL) were added HATU (286.9 mg, 0.754 mmol, 1.5 equiv) and DIEA (195.0 mg, 1.509 mmol, 3 equiv) at room temperature. The resulting mixture was stirred for 3 h at room temperature. The resulting mixture was diluted with water (50 mL) and extracted with EtOAc (3×30 mL). The combined organic layers were washed with brine (2×100 mL), dried over anhydrous Na₂SO₄. After filtration, the filtrate was concentrated under reduced pressure. The residue was purified by reversed-phase flash chromatography to afford tert-butyl 6-(2-(2,6-dioxopiperidin-3-yl)-1-oxoisoindoline-5-carbonyl)-2,6-diazaspiro[3.3]heptane-2-carboxylate (120 mg, 51%) as a white solid. LC-MS (ES, m/z)=469.2 [M+1]⁺.

Step 2. Preparation of 3-(1-oxo-5-(2,6-diazaspiro[3.3]heptane-2-carbonyl)isoindolin-2-yl)piperidine-2,6-dione. To a stirred mixture of tert-butyl 6-(2-(2,6-dioxopiperidin-3-yl)-1-oxoisoindoline-5-carbonyl)-2,6-diazaspiro[3.3]heptane-2-carboxylate (120.0 mg, 0.256 mmol, 1 equiv) in DCM (1 mL) was added TFA (0.2 mL, 2.693 mmol, 10.51 equiv) dropwise at 0° C. The resulting mixture was stirred for 3 h at room temperature. The resulting mixture was diluted with water (50 mL) and extracted with EtOAc (3×30 mL). The combined organic layers were washed with brine (2×30 mL), dried over anhydrous Na₂SO₄. After filtration, the filtrate was concentrated under reduced pressure. The crude product (25 mg) was purified by Prep-HPLC to afford 3-(1-oxo-5-(2,6-diazaspiro[3.3]heptane-2-carbonyl)isoindolin-2-yl)piperidine-2,6-dione (3.6 mg, 3.82%) as a white semi-solid. LC-MS (ES, m/z)=369.0 [M+1]⁺. ¹H NMR (400 MHz, DMSO-d₆) δ 7.88 (d, J=7.9 Hz, 1H), 7.84 (s, 1H), 7.76 (dd, J=7.9, 1.4 Hz, 1H), 5.18 (dd, J=13.3, 5.2 Hz, 1H), 4.63-4.48 (m, 4H), 4.39 (s, 2H), 4.31-4.26 (m, 4H), 2.92 (ddd, J=18.4, 13.4, 5.4 Hz, 1H), 2.79 (ddd, J=17.6, 4.7, 2.4 Hz, 1H), 2.51 (qd, J=13.3, 4.7 Hz, 1H), 2.19 (dtd, J=12.8, 5.3, 2.4 Hz, 1H).

### Example 122

### Synthesis of 3-(5-(6,6-difluoro-2-azaspiro[3.3]heptane-2-carbonyl)-1-oxoisoindolin-2-yl)piperidine-2,6-dione

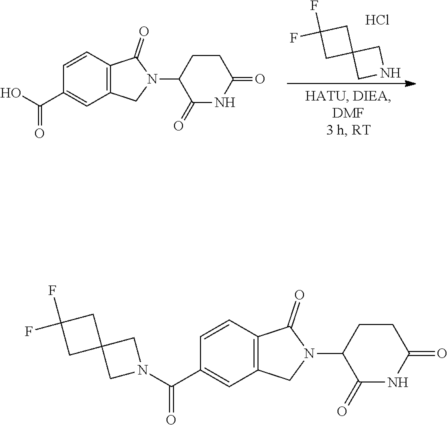 [SMILES: O=C1CCC(N2Cc3cc(C(=O)O)ccc3C2=O)C(=O)N1 ; FC1(F)CC2(CNC2)C1 ; Cl ; O=C1CCC(N2Cc3cc(C(=O)N4CC5(C4)CC(F)(F)C5)ccc3C2=O)C(=O)N1]

Step 1. Preparation of 3-(5-(6,6-difluoro-2-azaspiro[3.3]heptane-2-carbonyl)-1-oxoisoindolin-2-yl)piperidine-2,6-dione. A solution of 6,6-difluoro-2-azaspiro[3.3]heptane hydrochloride (90.0 mg, 0.531 mmol, 1 equiv), DIEA (205.8 mg, 1.593 mmol, 3 equiv) and 2-(2,6-dioxopiperidin-3-yl)-1-oxo-3H-isoindole-5-carboxylic acid (152.9 mg, 0.531 mmol, 1 equiv) in DMF (1.5 mL) was stirred for 5 min at room temperature. To the above mixture was added HATU (302.7 mg, 0.796 mmol, 1.5 equiv) at room temperature. The resulting mixture was stirred for an additional 3 h at room temperature. The resulting mixture was diluted with water (50 mL) and extracted with EtOAc (3×50 mL). The combined organic layers were washed with brine (2×100 mL), dried over anhydrous Na₂SO₄. After filtration, the filtrate was concentrated under reduced pressure. The crude product (40.0 mg) was purified by Prep-HPLC to afford 3-(5-(6,6-difluoro-2-azaspiro[3.3]heptane-2-carbonyl)-1-oxoisoindolin-2-yl)piperidine-2,6-dione (27.6 mg, 13%) as an off-white solid. LC-MS (ES, m/z)=404.15 [M+1]⁺. ¹H NMR (400 MHz, DMSO-d₆) δ 11.01 (s, 1H), 7.98-7.55 (m, 3H), 5.14 (dd, J=13.3, 5.1 Hz, 1H), 4.59-4.29 (m, 4H), 4.18 (s, 2H), 2.89 (dtd, J=29.1, 13.1, 12.5, 6.6 Hz, 5H), 2.61 (ddd, J=17.3, 4.5, 2.3 Hz, 1H), 2.41 (qd, J=13.2, 4.4 Hz, 1H), 2.10-1.97 (m, 1H).

### Example 123

### Synthesis of 3-(5-(6-fluoro-3-isopropylindoline-1-carbonyl)-1-oxoisoindolin-2-yl)piperidine-2,6-dione

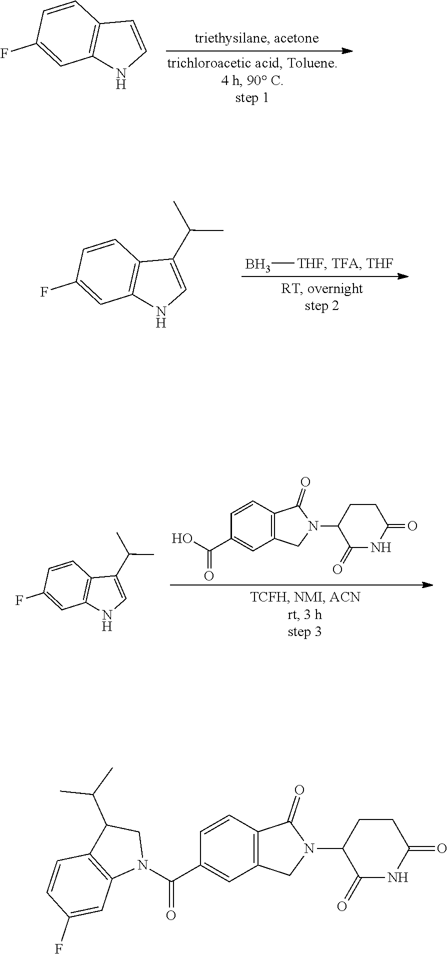

Step 1. Preparation of 6-fluoro-3-isopropyl-1H-indole. A solution of trichloroacetic acid (3.63 g, 22.198 mmol, 1.5 equiv), acetone (1.03 g, 17.759 mmol, 1.2 equiv) in methylbenzene (10 mL) was treated with triethylsilane (5.16 g, 44.397 mmol, 3 equiv) for 5 min at room temperature under nitrogen atmosphere followed by the addition of 6-fluoro-1H-indole (2.0 g, 14.799 mmol, 1 equiv) at 70° C. The resulting mixture was stirred for 4 h at 90° C. under nitrogen atmosphere. The resulting mixture was diluted with water (150 mL) and extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford 6-fluoro-3-isopropyl-1H-indole (1.0 g, 38%) as a yellow oil. LC-MS (ES, m/z)=178.2 [M+1]⁺.

Step 2. Preparation of 6-fluoro-3-isopropylindoline. A solution of 6-fluoro-3-isopropyl-1H-indole (500.0 mg, 2.821 mmol, 1 equiv) in THF (10 mL) was treated with BH₃-THF (8.46 mL, 8.463 mmol, 3 equiv) for 1 h at 0° C. under nitrogen atmosphere followed by the addition of TFA (8.5 mL, 114.420 mmol, 40.56 equiv) dropwise at 0° C. The resulting mixture was stirred overnight at room temperature. The reaction was quenched by the addition of MeOH (10 mL) at 0° C. The resulting mixture was concentrated under reduced pressure. The residue was purified by reversed-phase flash chromatography to afford 6-fluoro-3-isopropylindoline (500.0 mg, 99%) as a brown solid. LC-MS (ES, m/z)=180.2 [M+1]⁺.

Step 3. Preparation of 3-(5-(6-fluoro-3-isopropylindoline-1-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (200.0 mg, 0.694 mmol, 1 equiv) and 6-fluoro-3-isopropyl-2,3-dihydro-1H-indole (124.4 mg, 0.694 mmol, 1 equiv) in ACN (4 mL) were added TCFH (292.0 mg, 1.041 mmol, 1.5 equiv) and NMI (227.9 mg, 2.776 mmol, 4 equiv) at room temperature. The resulting mixture was stirred overnight at room temperature. The resulting mixture was diluted with water (100 mL) and extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford 3-(5-(6-fluoro-3-isopropylindoline-1-carbonyl)-1-oxoisoindolin-2-yl)piperidine-2,6-dione (25.4 mg, 8.14%) as an off-white solid. LC-MS (ES, m/z)=450.2 [M+1]⁺. ¹H NMR (400 MHz, DMSO-d₆) δ 11.01 (s, 1H), 7.89-7.82 (m, 4H), 7.31 (dd, J=8.3, 5.7 Hz, 1H), 6.93 (t, J=8.9 Hz, 1H), 5.16 (dd, J=13.3, 5.1 Hz, 1H), 4.55 (dd, J=17.7, 4.4 Hz, 1H), 4.42 (dd, J=17.6, 5.8 Hz, 2H), 4.13 (ddd, J=11.0, 9.5, 6.7 Hz, 1H), 3.72 (s, 1H), 3.28 (d, J=4.5 Hz, 1H), 2.93 (ddd, J=17.2, 13.5, 5.4 Hz, 1H), 2.66-2.57 (m, 1H), 2.44-2.35 (m, 2H), 2.07-1.96 (m, 3H), 0.81-0.70 (m, 3H).

### Example 124

### Synthesis of 3-(5-(6-fluoro-3-methylindoline-1-carbonyl)-1-oxoisoindolin-2-yl)piperidine-2,6-dione

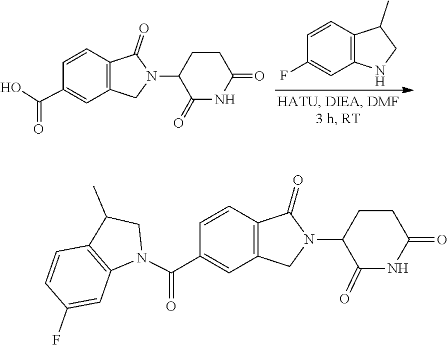

Step 1. Preparation of 3-(5-(6-fluoro-3-methylindoline-1-carbonyl)-1-oxoisoindolin-2-yl)piperidine-2,6-dione. A solution of 6-fluoro-3-methyl-2,3-dihydro-1H-indole (70.0 mg, 0.463 mmol, 1 equiv), DIEA (179.5 mg, 1.389 mmol, 3 equiv) and 2-(2,6-dioxopiperidin-3-yl)-1-oxo-3H-isoindole-5-carboxylic acid (133.5 mg, 0.463 mmol, 1 equiv) in DMF (1.5 mL) was stirred for 3 min at room temperature. To the above mixture was added HATU (264.1 mg, 0.695 mmol, 1.5 equiv) at room temperature. The resulting mixture was stirred for an additional 3 h at room temperature. The resulting mixture was diluted with water (20 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(6-fluoro-3-methylindoline-1-carbonyl)-1-oxoisoindolin-2-yl)piperidine-2,6-dione (24 mg, 12%) as an off-white solid. LC-MS (ES, m/z)=422.25 [M+1]⁺. ¹H NMR (400 MHz, DMSO-d₆) δ 11.02 (s, 1H), 7.85 (d, J=8.2 Hz, 3H), 7.73 (d, J=7.8 Hz, 1H), 7.31 (ddd, J=8.3, 5.8, 1.1 Hz, 1H), 6.93 (t, J=8.3 Hz, 1H), 5.16 (dd, J=13.3, 5.0 Hz, 1H), 4.55 (dd, J=17.6, 6.8 Hz, 1H), 4.45-4.31 (m, 1H), 4.19 (t, J=9.9 Hz, 1H), 3.67-3.53 (m, 1H), 3.41 (q, J=7.4 Hz, 1H), 2.93 (ddd, J=17.2, 13.6, 5.4 Hz, 1H), 2.68-2.54 (m, 1H), 2.45-2.35 (m, 1H), 2.11-1.95 (m, 1H), 1.23 (d, J=6.7 Hz, 3H).

### Example 125

### Synthesis of 1-(7-(isoindoline-2-carbonyl)naphthalen-2-yl)dihydropyrimidine-2,4(1H,3H)-dione

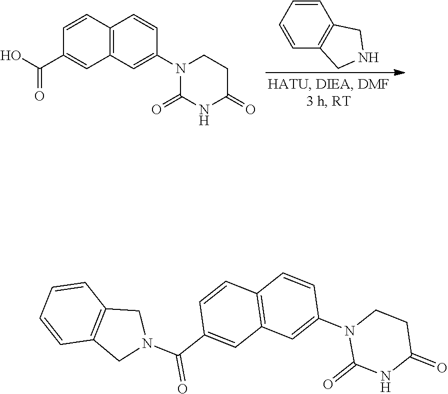 [SMILES: O=C1CCN(c2ccc3ccc(C(=O)O)cc3c2)C(=O)N1 ; c1ccc2c(c1)CNC2 ; O=C1CCN(c2ccc3ccc(C(=O)N4Cc5ccccc5C4)cc3c2)C(=O)N1]

Step 1. Preparation of 1-(7-(isoindoline-2-carbonyl)naphthalen-2-yl)dihydropyrimidine-2,4(1H,3H)-dione. A solution of isoindoline (100.6 mg, 0.843 mmol, 3 equiv), DIEA (109.1 mg, 0.843 mmol, 3 equiv) and 7-(2,4-dioxo-1,3-diazinan-1-yl)naphthalene-2-carboxylic acid (80.0 mg, 0.281 mmol, 1 equiv) in DMF (3 mL) was stirred for 3 min at room temperature. To the above mixture was added HATU (160.5 mg, 0.422 mmol, 1.5 equiv) at room temperature. The resulting mixture was stirred for an additional 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 1-(7-(isoindoline-2-carbonyl)naphthalen-2-yl)dihydropyrimidine-2,4(1H,3H)-dione (50 mg, 46%) as an off-white solid. LC-MS (ES, m/z)=386.2 [M+1]⁺. ¹H NMR (400 MHz, DMSO-d₆) δ 10.49 (s, 1H), 8.22 (d, J=1.6 Hz, 1H), 8.01 (dd, J=8.7, 6.5 Hz, 2H), 7.94 (d, J=2.2 Hz, 1H), 7.67 (ddd, J=13.6, 8.6, 1.9 Hz, 2H), 7.43 (d, J=7.1 Hz, 1H), 7.37-7.24 (m, 3H), 4.90 (d, J=23.7 Hz, 4H), 3.94 (t, J=6.6 Hz, 2H), 2.78 (t, J=6.6 Hz, 2H).

### Example 126

### Synthesis of 1-(7-(4-fluoroindoline-1-carbonyl)naphthalen-2-yl)dihydropyrimidine-2,4(1H,3H)-dione

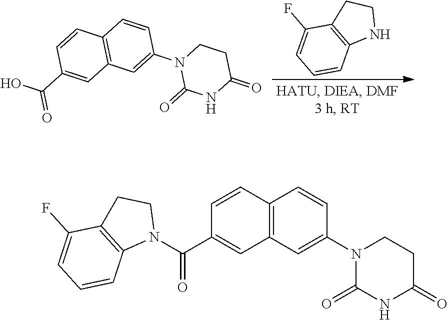 [SMILES: O=C1CCN(c2ccc3ccc(C(=O)O)cc3c2)C(=O)N1 ; Fc1cccc2c1CCN2 ; O=C1CCN(c2ccc3ccc(C(=O)N4CCc5c(F)cccc54)cc3c2)C(=O)N1]

Step 1. Preparation of 1-(7-(4-fluoroindoline-1-carbonyl)naphthalen-2-yl)dihydropyrimidine-2,4(1H,3H)-dione. A solution of 4-fluoro-2,3-dihydro-1H-indole (115.8 mg, 0.843 mmol, 3 equiv), DIEA (109.1 mg, 0.843 mmol, 3 equiv) and 7-(2,4-dioxo-1,3-diazinan-1-yl)naphthalene-2-carboxylic acid (80.0 mg, 0.281 mmol, 1 equiv) in DMF (2 mL) was stirred for 10 min at room temperature. To the above mixture was added HATU (160.5 mg, 0.422 mmol, 1.5 equiv) at room temperature. The resulting mixture was stirred for an additional 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The crude product (90.0 mg) was purified by Prep-HPLC to afford 1-(7-(4-fluoroindoline-1-carbonyl)naphthalen-2-yl)dihydropyrimidine-2,4(1H,3H)-dione (7.0 mg, 6.17%) as an off-white solid. LC-MS (ES, m/z)=404.1 [M+1]. ¹H NMR (400 MHz, DMSO-d₆) δ 10.49 (s, 1H), 8.22-7.55 (m, 7H), 7.23 (s, 1H), 6.95-6.83 (m, 1H), 4.15 (t, J=8.3 Hz, 2H), 3.94 (t, J=6.6 Hz, 2H), 3.13 (t, J=8.2 Hz, 2H), 2.77 (t, J=6.7 Hz, 2H), 1.36 (s, 1H).

### Example 127

### Synthesis of 1-(7-(4-fluoroisoindoline-2-carbonyl)naphthalen-2-yl)dihydropyrimidine-2,4(1H,3H)-dione

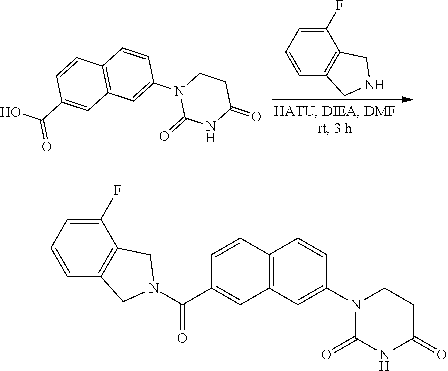 [SMILES: O=C1CCN(c2ccc3ccc(C(=O)O)cc3c2)C(=O)N1 ; Fc1cccc2c1CNC2 ; O=C1CCN(c2ccc3ccc(C(=O)N4Cc5cccc(F)c5C4)cc3c2)C(=O)N1]

Step 1. Preparation of 1-(7-(4-fluoroisoindoline-2-carbonyl)naphthalen-2-yl)dihydropyrimidine-2,4(1H,3H)-dione. To a stirred mixture of 4-fluoro-2,3-dihydro-1H-isoindole (48.0 mg, 0.350 mmol, 1 equiv) and 7-(2,4-dioxo-1,3-diazinan-1-yl)naphthalene-2-carboxylic acid (99.5 mg, 0.350 mmol, 1 equiv) in DMF (1.5 mL) were added DIEA (135.7 mg, 1.050 mmol, 3 equiv) and HATU (199.6 mg, 0.525 mmol, 1.5 equiv) at room temperature. The resulting mixture was stirred for 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 1-(7-(4-fluoroisoindoline-2-carbonyl)naphthalen-2-yl)dihydropyrimidine-2,4(1H,3H)-dione (40 mg, 28%) as a white solid. LC-MS (ES, m/z)=404.1 [M+1]⁺. ¹H NMR (400 MHz, DMSO-d₆) δ 10.47 (s, 1H), 8.23 (dd, J=3.8, 1.7 Hz, 1H), 8.01-7.94 (t, J=11.7 Hz, 3H), 7.67 (ddt, J=18.7, 8.8, 1.8 Hz, 2H), 7.40-7.28 (m, 1H), 7.20-7.07 (m, 2H), 4.92 (d, J=8.8 Hz, 4H), 3.95 (td, J=6.7, 3.3 Hz, 2H), 2.78 (t, J=6.6 Hz, 2H).

### Example 128

### Synthesis of 1-(7-(1,2,3,4-tetrahydroquinoline-1-carbonyl)naphthalen-2-yl)dihydropyrimidine-2,4(1H,3H)-dione

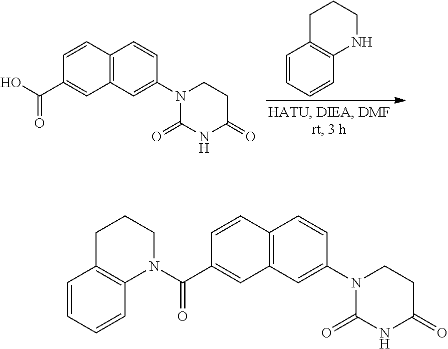 [SMILES: O=C1CCN(c2ccc3ccc(C(=O)O)cc3c2)C(=O)N1 ; c1ccc2c(c1)CCCN2 ; O=C1CCN(c2ccc3ccc(C(=O)N4CCCc5ccccc54)cc3c2)C(=O)N1]

Step 1. Preparation of 1-(7-(1,2,3,4-tetrahydroquinoline-1-carbonyl)naphthalen-2-yl)dihydropyrimidine-2,4(1H,3H)-dione. To a stirred mixture of 7-(2,4-dioxo-1,3-diazinan-1-yl)naphthalene-2-carboxylic acid (100.0 mg, 0.352 mmol, 1 equiv) and tetrahydroquinoline (93.7 mg, 0.704 mmol, 2 equiv) in DMF (1.5 mL) were added HATU (200.7 mg, 0.528 mmol, 1.5 equiv) and DIEA (136.4 mg, 1.056 mmol, 3 equiv) at room temperature. The resulting mixture was stirred for 3 h at room temperature. The resulting mixture was diluted with water (50 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford 1-(7-(1,2,3,4-tetrahydroquinoline-1-carbonyl)naphthalen-2-yl)dihydropyrimidine-2,4(1H,3H)-dione (14 mg, 9.68%) as a white solid. LC-MS (ES, m/z)=400.1 [M+1]⁺. ¹H NMR (400 MHz, DMSO-d₆) δ 10.46 (s, 1H), 7.97 (d, J=1.6 Hz, 1H), 7.91 (d, J=8.9 Hz, 1H), 7.87-7.80 (m, 2H), 7.59 (dd, J=8.8, 2.1 Hz, 1H), 7.33 (dd, J=8.3, 1.7 Hz, 1H), 7.25-7.19 (m, 1H), 6.98 (td, J=7.4, 1.4 Hz, 1H), 6.88-6.77 (m, 2H), 3.90 (t, J=6.6 Hz, 2H), 3.81 (t, J=6.4 Hz, 2H), 2.86 (t, J=6.6 Hz, 2H), 2.75 (t, J=6.6 Hz, 2H), 1.99 (p, J=6.5 Hz, 2H).

### Example 129

### Synthesis of 1-(7-(1,2,3,4-tetrahydroisoquinoline-2-carbonyl)naphthalen-2-yl)dihydropyrimidine-2,4(1H,3H)-dione

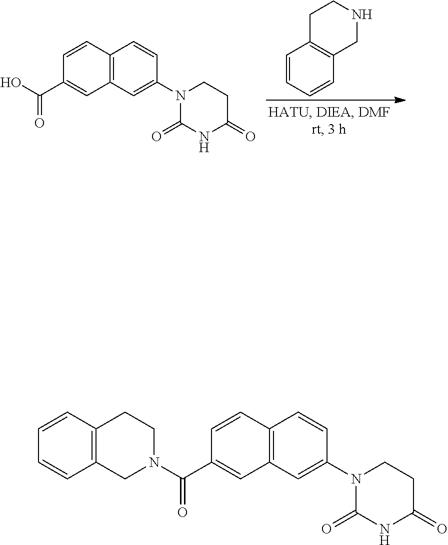 [SMILES: O=C1CCN(c2ccc3ccc(C(=O)O)cc3c2)C(=O)N1 ; c1ccc2c(c1)CCNC2 ; O=C1CCN(c2ccc3ccc(C(=O)N4CCc5ccccc5C4)cc3c2)C(=O)N1]

Step 1. Preparation of 1-(7-(1,2,3,4-tetrahydroisoquinoline-2-carbonyl)naphthalen-2-yl)dihydropyrimidine-2,4(1H,3H)-dione. A solution of tetrahydroisoquinoline (74.9 mg, 0.562 mmol, 2 equiv), DIEA (109.1 mg, 0.843 mmol, 3 equiv) and 7-(2,4-dioxo-1,3-diazinan-1-yl)naphthalene-2-carboxylic acid (80.0 mg, 0.281 mmol, 1.00 equiv) in DMF (2 mL) was stirred for 3 min at room temperature. To the above mixture was added HATU (160.5 mg, 0.422 mmol, 1.5 equiv) at room temperature. The resulting mixture was stirred for additional 3 h at room temperature. The resulting mixture was diluted with water (50 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford 1-(7-(1,2,3,4-tetrahydroisoquinoline-2-carbonyl)naphthalen-2-yl)dihydropyrimidine-2,4(1H,3H)-dione (5.8 mg, 5.16%) as an off-white solid. LC-MS (ES, m/z)=400.2 [M+1]⁺. ¹H NMR (400 MHz, DMSO-d₆) δ 10.48 (s, 1H), 8.06-7.79 (m, 4H), 7.70-7.48 (m, 2H), 7.19 (d, J=3.3 Hz, 4H), 4.72 (d, J=75.2 Hz, 2H), 3.93 (t, J=6.6 Hz, 2H), 3.61 (s, 1H), 2.90 (s, 2H), 2.76 (d, J=6.8 Hz, 2H).

### Example 130

### Synthesis of 3-(5-(4-methylisoindoline-2-carbonyl)-1-oxoisoindolin-2-yl)piperidine-2,6-dione

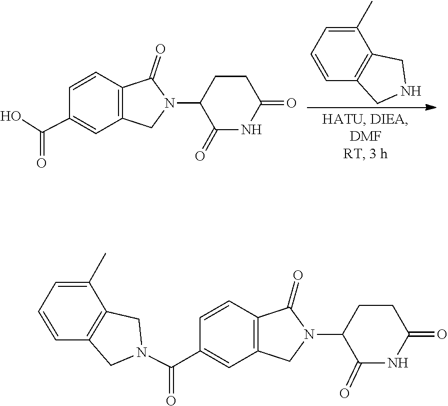

Step 1. Preparation of 3-(5-(4-methylisoindoline-2-carbonyl)-1-oxoisoindolin-2-yl)piperidine-2,6-dione. To a stirred solution of 4-methyl-2,3-dihydro-1H-isoindole (50.0 mg, 0.38 mmol, 1 equiv) and 2-(2,6-dioxopiperidin-3-yl)-1-oxo-3H-isoindole-5-carboxylic acid (129.9 mg, 0.450 mmol, 1.2 equiv) in DMF (1.5 mL) were added DIEA (145.6 mg, 1.13 mmol, 3.0 equiv) and HATU (171.3 mg, 0.450 mmol, 1.2 equiv) at room temperature. The resulting mixture was stirred for 3 h at room temperature under air atmosphere. The resulting mixture was diluted with water (50 mL). The resulting mixture was extracted with EtOAc (2×30 mL). The combined organic layers were washed with water (2×50 mL), dried over anhydrous Na₂SO₄. After filtration, the filtrate was concentrated under reduced pressure. The crude product (50.0 mg) was purified by Prep-HPLC to afford 3-(5-(4-methylisoindoline-2-carbonyl)-1-oxoisoindolin-2-yl)piperidine-2,6-dione (5.0 mg, 3.14%) as a white solid. LC-MS (ES, m/z)=404.1 [M+1]⁺. ¹H NMR (400 MHz, DMSO-d₆) δ 10.99 (s, 1H) 7.83 (d, J=11.0 Hz, 2H), 7.73 (t, J=7.9 Hz, 1H), 7.14-7.03 (m, 3H), 5.15 (dd, J=13.3, 5.1 Hz, 1H), 4.90 (s, 2H), 4.85 (s, 2H), 4.74 (d, J=16.8 Hz, 1H), 4.58-4.49 (m, 1H), 2.93 (s, 1H), 2.62 (d, J=17.4 Hz, 1H), 2.47-2.39 (m, 1H), 2.30 (s, 2H), 2.12 (s, 1H), 2.05 (s, 1H).

### Example 131

### Synthesis of 2-(2-(2,6-dioxopiperidin-3-yl)-1-oxoisoindoline-5-carbonyl)isoindoline-4-carbonitrile

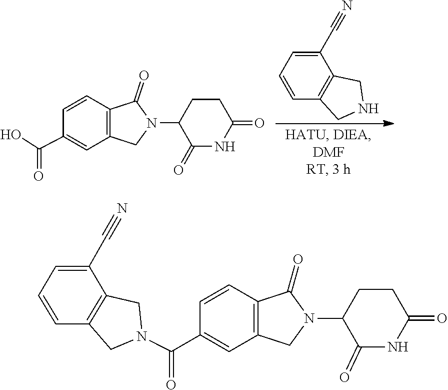

Step 1. Preparation of 2-(2-(2,6-dioxopiperidin-3-yl)-1-oxoisoindoline-5-carbonyl)isoindoline-4-carbonitrile. To a stirred solution of 2,3-dihydro-1H-isoindole-4-carbonitrile (50.0 mg, 0.347 mmol, 1 equiv) and 2-(2,6-dioxopiperidin-3-yl)-1-oxo-3H-isoindole-5-carboxylic acid (120.0 mg, 0.416 mmol, 1.2 equiv) in DMF (1 mL) were added DIEA (134.5 mg, 1.041 mmol, 3 equiv) and HATU (158.3 mg, 0.416 mmol, 1.2 equiv) at room temperature. The resulting mixture was stirred for 3 h at room temperature. The resulting mixture was diluted with water (50 mL). The resulting mixture was extracted with EtOAc (3×30 mL). The combined organic layers were washed with water (3×50 mL), dried over anhydrous Na₂SO₄. After filtration, the filtrate was concentrated under reduced pressure. The crude product (50.0 mg) was purified by Prep-HPLC to afford 2-(2-(2,6-dioxopiperidin-3-yl)-1-oxoisoindoline-5-carbonyl)isoindoline-4-carbonitrile (14 mg, 9.33%) as a white solid. LC-MS (ES, m/z)=415.1 [M+1]⁺. ¹H NMR (400 MHz, DMSO-d₆) δ 11.01 (s, 1H), 7.89-7.62 (m, 5H), 7.52 (dt, J=15.0, 7.6 Hz, 1H), 5.16 (dd, J=13.3, 5.2 Hz, 1H), 5.05 (s, 1H), 4.97 (s, 2H), 4.86 (s, 1H), 4.55 (dd, J=17.6, 8.5 Hz, 1H), 4.42 (dd, J=17.5, 7.3 Hz, 1H), 2.93 (ddd, J=18.2, 13.7, 5.5 Hz, 1H), 2.62 (d, J=16.7 Hz, 1H), 2.42 (s, 1H), 2.05 (s, 1H).

### Examples 132 and 133

### Synthesis of 3-(5-((R)-6-fluoro-3-methylindoline-1-carbonyl)-1-oxoisoindolin-2-yl)piperidine-2,6-dione and 3-(5-((S)-6-fluoro-3-methylindoline-1-carbonyl)-1-oxoisoindolin-2-yl)piperidine-2,6-dione

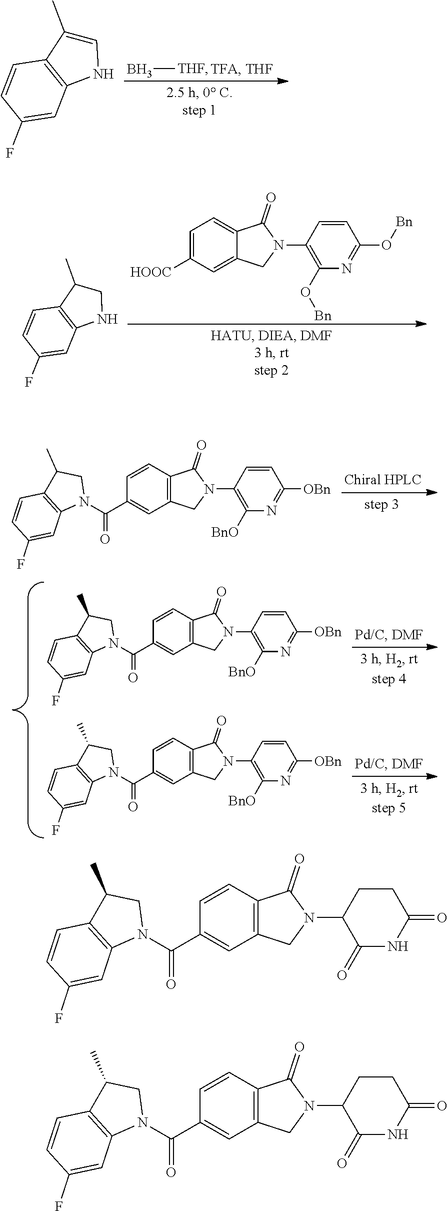

Step 1. Preparation of 6-fluoro-3-methylindoline. A solution of 6-fluoro-3-methyl-1H-indole (1 g, 6.704 mmol, 1 equiv) in THF (10 mL) was treated with TFA (7.64 g, 67.040 mmol, 10 equiv) for 10 min at 0° C. under nitrogen atmosphere followed by the addition of BH₃-THF (50 mL, 50.012 mmol, 7.46 equiv) dropwise at 0° C. The resulting mixture was stirred for 2.5 h at room temperature under nitrogen atmosphere. The reaction was quenched with Water at 0° C. and extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford 6-fluoro-3-methylindoline (900 mg, 89%) as a white oil. LC-MS (ES, m/z)=152.0 [M+1]⁺.

Step 2. Preparation of 2-(2,6-bis(benzyloxy)pyridin-3-yl)-5-(6-fluoro-3-methylindoline-1-carbonyl)isoindolin-1-one. To a stirred mixture of 2-[2,6-bis(benzyloxy)pyridin-3-yl]-1-oxo-3H-isoindole-5-carboxylic acid (400.0 mg, 0.857 mmol, 1 equiv) and 6-fluoro-3-methylindoline (194.4 mg, 1.285 mmol, 1.5 equiv) in DMF (8.00 mL) were added HATU (489.0 mg, 1.285 mmol, 1.5 equiv) and DIEA (332.4 mg, 2.571 mmol, 3 equiv) at room temperature. The resulting mixture was stirred for 3 h at room temperature. The resulting mixture was diluted with water (100 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(6-fluoro-3-methylindoline-1-carbonyl)isoindolin-1-one (500 mg, 97%) as a brown oil. LC-MS (ES, m/z)=600.0 [M+1]⁺.

Step 3. Preparation of (R)-2-(2,6-bis(benzyloxy)pyridin-3-yl)-5-(6-fluoro-3-methylindoline-1-carbonyl)isoindolin-1-one and (S)-2-(2,6-bis(benzyloxy)pyridin-3-yl)-5-(6-fluoro-3-methylindoline-1-carbonyl)isoindolin-1-one. The product (400.0 mg) was purified by Chiral HPLC with the following conditions (Column: CHIRAL ART Cellulose-SB, 3*25 cm, 5 μm; Mobile Phase A: Hex (10 mM NH₃-MeOH), Mobile Phase B: EtOH-HPLC; Flow rate: 40 mL/min; Gradient: isocratic 50; Wave Length: 210/220 nm; RT1 (min): 11.9; RT2 (min): 19.3; Sample Solvent: EtOH:DCM=2:1; Injection Volume: 0.8 mL; Number Of Runs: 7) to afford (R)-2-(2,6-bis(benzyloxy)pyridin-3-yl)-5-(6-fluoro-3-methylindoline-1-carbonyl)isoindolin-1-one (160 mg, 43%) as a yellow oil and (S)-2-(2,6-bis(benzyloxy)pyridin-3-yl)-5-(6-fluoro-3-methylindoline-1-carbonyl)isoindolin-1-one (170.0 mg, 44%) as a yellow oil.

Step 4. Preparation of 3-(5-((R)-6-fluoro-3-methylindoline-1-carbonyl)-1-oxoisoindolin-2-yl)piperidine-2,6-dione. To a stirred mixture of (R)-2-(2,6-bis(benzyloxy)pyridin-3-yl)-5-(6-fluoro-3-methylindoline-1-carbonyl)isoindolin-1-one (150.0 mg, 0.250 mmol, 1 equiv) in DMF (2 mL) was added Palladium 10% on Carbon (wetted with ca. 55% Water) (75.0 mg) at room temperature. The resulting mixture was stirred for 3 h at room temperature under hydrogen atmosphere. The resulting mixture was filtered, the filter cake was washed with THF (3×5 mL). The filtrate was concentrated under reduced pressure. The residue was purified by reversed-phase flash chromatography to afford 3-(5-((R)-6-fluoro-3-methylindoline-1-carbonyl)-1-oxoisoindolin-2-yl)piperidine-2,6-dione (65 mg, 61%) as a white solid. LC-MS (ES, m/z)=422.1 [M+1]⁺. ¹H NMR (400 MHz, DMSO-d₆) δ 11.02 (s, 1H), 8.14-7.61 (m, 4H), 7.36-7.27 (m, 1H), 6.97-6.88 (m, 1H), 5.16 (dd, J=13.3, 5.1 Hz, 1H), 4.54 (dd, J=17.6, 6.8 Hz, 1H), 4.41 (dd, J=17.6, 6.7 Hz, 1H), 4.19 (t, J=9.9 Hz, 1H), 3.62 (ddd, J=10.5, 7.1, 1.8 Hz, 1H), 3.42 (p, J=7.2 Hz, 1H), 2.93 (ddd, J=17.2, 13.5, 5.4 Hz, 1H), 2.66-2.57 (m, 1H), 2.41 (td, J=13.2, 4.5 Hz, 1H), 2.04 (dtd, J=10.0, 5.0, 2.5 Hz, 1H), 1.23 (d, J=6.7 Hz, 3H).

Step 5. Preparation of 3-(5-((S)-6-fluoro-3-methylindoline-1-carbonyl)-1-oxoisoindolin-2-yl)piperidine-2,6-dione. To a stirred mixture of (S)-2-(2,6-bis(benzyloxy)pyridin-3-yl)-5-(6-fluoro-3-methylindoline-1-carbonyl)isoindolin-1-one (150.0 mg, 0.250 mmol, 1 equiv) in DMF (2 mL) were added palladium 10% on Carbon (wetted with ca. 55% Water) (75 mg) at room temperature. The resulting mixture was stirred for 3 h at room temperature under hydrogen atmosphere. The resulting mixture was filtered, the filter cake was washed with THF (3×5 mL). The filtrate was concentrated under reduced pressure. The residue was purified by reversed-phase flash chromatography to afford 3-(5-((S)-6-fluoro-3-methylindoline-1-carbonyl)-1-oxoisoindolin-2-yl)piperidine-2,6-dione (71 mg, 68%) as a white solid. LC-MS (ES, m/z)=422.1 [M+1]⁺. ¹H NMR (400 MHz, DMSO-d₆) δ 11.02 (s, 1H), 7.79 (dd, J=44.9, 8.0 Hz, 4H), 7.31 (ddd, J=8.3, 5.8, 1.1 Hz, 1H), 6.98-6.88 (m, 1H), 5.16 (dd, J=13.3, 5.1 Hz, 1H), 4.54 (dd, J=17.6, 6.8 Hz, 1H), 4.41 (dd, J=17.6, 6.7 Hz, 1H), 4.19 (t, J=9.8 Hz, 1H), 3.62 (ddd, J=10.6, 7.1, 1.7 Hz, 1H), 3.42 (p, J=7.3 Hz, 1H), 2.93 (ddd, J=17.1, 13.6, 5.4 Hz, 1H), 2.67-2.57 (m, 1H), 2.41 (td, J=13.2, 4.5 Hz, 1H), 2.04 (ddq, J=10.4, 5.4, 3.3, 2.6 Hz, 1H), 1.23 (d, J=6.7 Hz, 3H).

### Example 134

### Synthesis of 1-(7-(6-fluoroindoline-1-carbonyl)-1-methylnaphthalen-2-yl)dihydropyrimidine-2,4(1H,3H)-dione

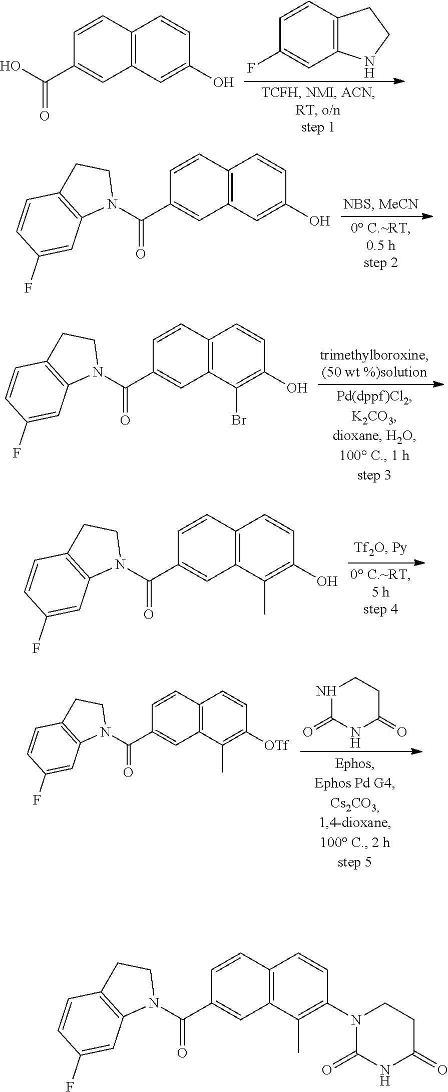

Step 1. Preparation of (6-fluoroindolin-1-yl)(7-hydroxynaphthalen-2-yl)methanone. To a stirred solution of 7-hydroxynaphthalene-2-carboxylic acid (2.0 g, 10.628 mmol, 1 equiv), 6-fluoro-2,3-dihydro-1H-indole (1.7 g, 12.754 mmol, 1.2 equiv) and NMI (3.5 g, 42.512 mmol, 4 equiv) in ACN (10 mL) was added TCFH (4.5 g, 15.942 mmol, 1.5 equiv) in portions at room temperature under nitrogen atmosphere. The resulting mixture was stirred overnight at room temperature under nitrogen atmosphere. The resulting mixture was concentrated under reduced pressure. The resulting mixture was diluted with water (300 mL). The resulting mixture was extracted with EtOAc (3×200 mL). The combined organic layers were washed with brine (2×300 mL), dried over anhydrous Na₂SO₄. After filtration, the filtrate was concentrated under reduced pressure. The residue was purified by reversed-phase flash chromatography to afford (6-fluoroindolin-1-yl)(7-hydroxynaphthalen-2-yl)methanone (1.5 g, 46%) as a yellow solid. LC-MS (ES, m/z)=308.2 [M+1]⁺.

Step 2. Preparation of (8-bromo-7-hydroxynaphthalen-2-yl)(6-fluoroindolin-1-yl)methanone. To a stirred solution of (6-fluoroindolin-1-yl)(7-hydroxynaphthalen-2-yl)methanone (1.0 g, 3.254 mmol, 1 equiv) in ACN (10 mL) was added NBS (580 mg, 3.254 mmol, 1 equiv) in portions at 0° C. under nitrogen atmosphere. The resulting mixture was stirred for 0.5 h at room temperature under nitrogen atmosphere. The resulting mixture was diluted with water (100 mL). The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by reversed-phase flash chromatography to afford (8-bromo-7-hydroxynaphthalen-2-yl)(6-fluoroindolin-1-yl)methanone (750 mg, 60%) as a yellow solid. LC-MS (ES, m/z)=386.0 [M+1]⁺.

Step 3. Preparation of (6-fluoroindolin-1-yl)(7-hydroxy-8-methylnaphthalen-2-yl)methanone. To a stirred solution of (8-bromo-7-hydroxynaphthalen-2-yl)(6-fluoroindolin-1-yl)methanone (600.0 mg, 1.554 mmol, 1 equiv) and 2,4,6-trimethyl-1,3,5,2,4,6-trioxatriborinane (585.1 mg, 4.662 mmol, 3 equiv) in dioxane (6 mL) and H₂O (0.6 mL) were added Pd(dppf)Cl₂ (113.7 mg, 0.155 mmol, 0.1 equiv) and K₂CO₃ (644.1 mg, 4.662 mmol, 3 equiv) at room temperature under nitrogen atmosphere. The resulting mixture was stirred for 1 h at 100° C. under nitrogen atmosphere. The mixture was allowed to cool down to room temperature. The resulting mixture was diluted with water (100 mL). The resulting mixture was extracted with EtOAc (3×100 mL). The combined organic layers were washed with brine (2×300 mL), dried over anhydrous Na₂SO₄. After filtration, the filtrate was concentrated under reduced pressure. The residue was purified by reversed-phase flash chromatography to afford (6-fluoroindolin-1-yl)(7-hydroxy-8-methylnaphthalen-2-yl)methanone (400 mg, 80%) as a brown solid. LC-MS (ES, m/z)=322.1 [M+1]⁺.

Step 4. Preparation of 7-(6-fluoroindoline-1-carbonyl)-1-methylnaphthalen-2-yl trifluoromethanesulfonate. To a stirred solution of (6-fluoroindolin-1-yl)(7-hydroxy-8-methylnaphthalen-2-yl)methanone (400.0 mg, 2.178 mmol, 1 equiv) in pyridine (7 mL) was added Tf₂O (721.8 mg, 3.267 mmol, 1.5 equiv) dropwise at 0° C. under nitrogen atmosphere. The resulting mixture was stirred for 5 h at room temperature under nitrogen atmosphere. The reaction was quenched by the addition of Water/Ice (20 mL) at 0° C. The resulting mixture was diluted with water (50 mL). The resulting mixture was extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford 7-(6-fluoroindoline-1-carbonyl)-1-methylnaphthalen-2-yl trifluoromethanesulfonate (280 mg, 43%) as a yellow solid. LC-MS (ES, m/z)=454.1 [M+1]⁺.

Step 5. Preparation of 1-(7-(6-fluoroindoline-1-carbonyl)-1-methylnaphthalen-2-yl)dihydropyrimidine-2,4(1H,3H)-dione. To a stirred solution of 7-(6-fluoroindoline-1-carbonyl)-1-methylnaphthalen-2-yl trifluoromethanesulfonate (280.0 mg, 0.662 mmol, 1 equiv), 5,6-dihydrouracil (226.5 mg, 1.986 mmol, 3 equiv) and Cs₂CO₃ (646.7 mg, 1.986 mmol, 3 equiv) in dioxane (5 mL) were added EPhos (35.4 mg, 0.066 mmol, 0.1 equiv) and EPhos Pd G₄ (60.8 mg, 0.066 mmol, 0.1 equiv) at room temperature under nitrogen atmosphere. The resulting mixture was stirred for 2 h at 100° C. under nitrogen atmosphere. The mixture was allowed to cool down to room temperature. The resulting mixture was diluted with water (50 mL). The resulting mixture was extracted with EtOAc (3×50 mL). The combined organic layers were washed with brine (2×100 mL), dried over anhydrous Na₂SO₄. After filtration, the filtrate was concentrated under reduced pressure. The crude product (120.0 mg) was purified by Prep-HPLC to afford 1-(7-(6-fluoroindoline-1-carbonyl)-1-methylnaphthalen-2-yl)dihydropyrimidine-2,4(1H,3H)-dione (7.0 mg, 2.5%) as an off-white solid. LC-MS (ES, m/z)=418.2 [M+1]⁺. ¹H NMR (400 MHz, DMSO-d₆) δ 10.43 (s, 1H), 8.35 (s, 1H), 8.10-7.49 (m, 5H), 7.34-7.26 (m, 1H), 6.90 (d, J=9.5 Hz, 1H), 4.27-4.05 (m, 2H), 3.87 (ddd, J=12.4, 9.8, 5.2 Hz, 1H), 3.60 (dt, J=12.1, 5.9 Hz, 1H), 3.07 (t, J=8.3 Hz, 2H), 2.88 (ddd, J=16.1, 9.8, 6.0 Hz, 1H), 2.74 (dt, J=16.6, 5.5 Hz, 1H), 2.54 (s, 3H), 1.24 (s, 1H).

### Example 135

### Synthesis of 3-(5-(7-fluoro-1-methylisoindoline-2-carbonyl)-1-oxoisoindolin-2-yl)piperidine-2,6-dione

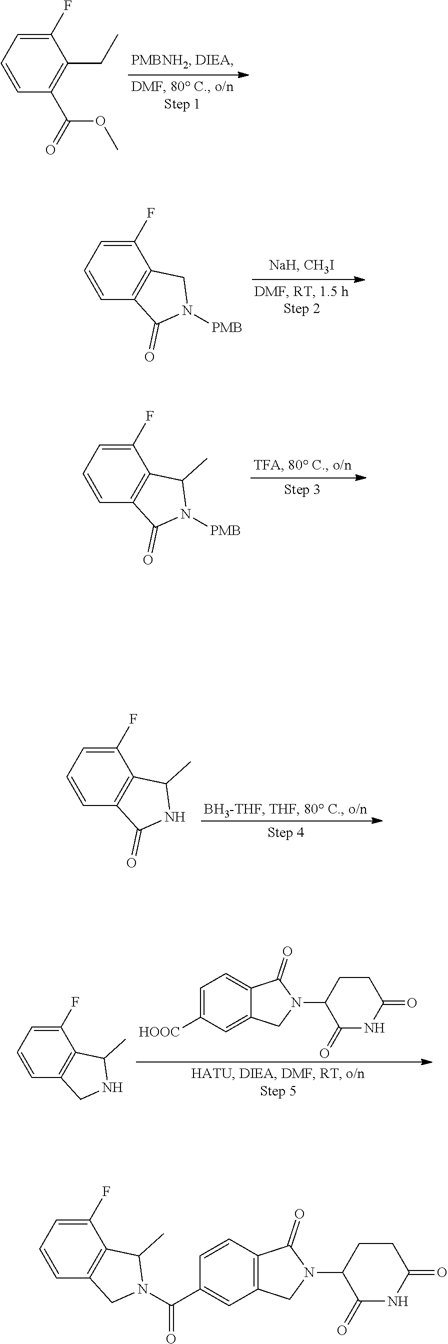

Step 1. Preparation of 4-fluoro-2-(4-methoxybenzyl)isoindolin-1-one. To a stirred solution of methyl 2-(bromomethyl)-3-fluorobenzoate (4.5 g, 18.214 mmol, 1 equiv) and (4-methoxyphenyl)methanamine (5.0 g, 36.428 mmol, 2 equiv) in DMF (90 mL) was added DIEA (7.06 g, 54.624 mmol, 3.00 equiv) at room temperature. The resulting mixture was stirred overnight at 80° C. The resulting mixture was diluted with water (300 mL). The resulting mixture was extracted with EtOAc (3×200 mL). The combined organic layers were washed with brine (3×300 mL), dried over anhydrous Na₂SO₄. After filtration, the filtrate was concentrated under reduced pressure. The residue was purified by reversed-phase flash chromatography to afford 4-fluoro-2-(4-methoxybenzyl)isoindolin-1-one (4.6 g, 93%) as a light yellow solid. LC-MS (ES, m/z)=272.1 [M+1]⁺.

Step 2. Preparation of 4-fluoro-2-(4-methoxybenzyl)-3-methylisoindolin-1-one. To a stirred solution of 4-fluoro-2-(4-methoxybenzyl)isoindolin-1-one (3.0 g, 11.058 mmol, 1 equiv) in DMF (60 mL) was added NaH (0.66 g, 16.587 mmol, 1.5 equiv, 60%) in portions at 0° C. under nitrogen atmosphere. The resulting mixture was stirred for 0.5 h at 0° C. under nitrogen atmosphere. To the above mixture was added CH₃I (2.04 g, 14.375 mmol, 1.3 equiv) dropwise at 0° C. The resulting mixture was stirred for an additional 1.5 h at room temperature. The reaction was quenched by the addition of Water/Ice (100 mL) at 0° C. The resulting mixture was extracted with EtOAc (3×200 mL). The combined organic layers were washed with brine (3×200 mL), dried over anhydrous Na₂SO₄. After filtration, the filtrate was concentrated under reduced pressure. The residue was purified by reversed-phase flash chromatography to afford 4-fluoro-2-(4-methoxybenzyl)-3-methylisoindolin-1-one (2.45 g, 78%) as a colorless oil. LC-MS (ES, m/z)=286.1 [M+1]⁺.

Step 3. Preparation of 4-fluoro-3-methylisoindolin-1-one. A solution of 4-fluoro-2-(4-methoxybenzyl)-3-methylisoindolin-1-one (2.9 g, 10.164 mmol, 1 equiv) in TFA (30 mL) was stirred overnight at 80° C. The resulting mixture was diluted with water (300 mL). The resulting mixture was extracted with CH₂Cl₁₋₂ (3×200 mL). The combined organic layers were washed with brine (3×300 mL), dried over anhydrous Na₂SO₄. After filtration, the filtrate was concentrated under reduced pressure. The residue was purified by reversed-phase flash chromatography to afford 4-fluoro-3-methylisoindolin-1-one (1.4 g, 83%) as an off-white solid. LC-MS (ES, m/z)=166.1 [M+1]⁺.

Step 4. Preparation of 7-fluoro-1-methylisoindoline. To a stirred solution of 4-fluoro-3-methylisoindolin-1-one (1.0 g, 6.054 mmol, 1 equiv) in THF (5 mL) was added BH₃-THF (30.27 mL, 30.270 mmol, 5 equiv) dropwise at 0° C. under nitrogen atmosphere. The resulting mixture was stirred for overnight at 80° C. under nitrogen atmosphere. The reaction was quenched with Water/Ice at 0° C. The mixture was acidified to pH 3 with 4M HCl. The resulting mixture was concentrated under reduced pressure. The residue was purified by reversed-phase flash chromatography to afford 7-fluoro-1-methylisoindoline (0.9 g, 98%) as a pink solid. LC-MS (ES, m/z)=152.1 [M+1]⁺.

Step 5. Preparation of 3-(5-(7-fluoro-1-methylisoindoline-2-carbonyl)-1-oxoisoindolin-2-yl)piperidine-2,6-dione. To a stirred solution of 2-(2,6-dioxopiperidin-3-yl)-1-oxo-3H-isoindole-5-carboxylic acid (150.0 mg, 0.520 mmol, 1 equiv) and 7-fluoro-1-methylisoindoline (157.3 mg, 1.040 mmol, 2 equiv) in DMF (3 mL) were added DIEA (336.3 mg, 2.600 mmol, 5 equiv) and HATU (296.8 mg, 0.780 mmol, 1.5 equiv) at room temperature. The resulting mixture was stirred overnight at room temperature. The resulting mixture was diluted with water (50 mL). The resulting mixture was extracted with CH₂Cl₂ (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford 3-(5-(7-fluoro-1-methylisoindoline-2-carbonyl)-1-oxoisoindolin-2-yl)piperidine-2,6-dione (138 mg, 63%) as a white solid. LC-MS (ES, m/z)=422.1 [M+1]⁺. ¹H NMR (400 MHz, DMSO-d₆) δ 11.03 (s, 1H), 7.84 (dd, J=8.4, 3.4 Hz, 2H), 7.72 (dt, J=7.6, 2.1 Hz, 1H), 7.34 (ddd, J=14.6, 10.0, 6.1 Hz, 1H), 7.20-7.06 (m, 2H), 5.62 (d, J=6.6 Hz, 1H), 5.16 (dd, J=13.2, 5.0 Hz, 1H), 5.01 (s, 1H), 4.64-4.33 (m, 3H), 3.03-2.81 (m, 1H), 2.74-2.55 (m, 1H), 2.44 (dd, J=13.1, 4.2 Hz, 1H), 2.11-1.95 (m, 1H), 1.66-1.04 (m, 3H).

### Example 136

### Synthesis of 3-(5-(4-fluoro-1-methylisoindoline-2-carbonyl)-1-oxoisoindolin-2-yl)piperidine-2,6-dione

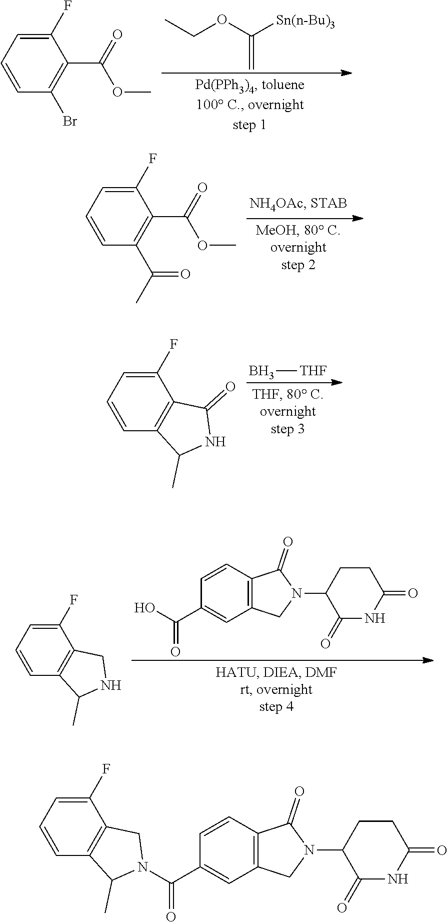

Step 1. Preparation of methyl 2-acetyl-6-fluorobenzoate. To a stirred solution of methyl 2-bromo-6-fluorobenzoate (3.0 g, 12.874 mmol, 1 equiv) and tributyl(1-ethoxyethenyl)stannane (5.6 g, 15.506 mmol, 1.20 equiv) in toluene (60 mL) was added Pd(PPh₃)₄ (1.5 g, 1.298 mmol, 0.10 equiv) at room temperature under nitrogen atmosphere. The resulting mixture was stirred for overnight at 100° C. under nitrogen atmosphere. The mixture was allowed to cool down to room temperature. To the above mixture was added HCl (20 mL, 1N) and stirred for 1 hour. The organic layer was dried over MgSO₄ and concentrated under reduced pressure. The residue was purified by silica gel column chromatography, eluted with PE/EA (3:1) to afford methyl 2-acetyl-6-fluorobenzoate (1.8 g, 71%) as a yellow oil. LC-MS (ES, m/z)=197.1 [M+1]⁺.

Step 2. Preparation of 7-fluoro-3-methylisoindolin-1-one. To a stirred mixture of methyl 2-acetyl-6-fluorobenzoate (1.0 g, 5.097 mmol, 1 equiv) and NH₄OAc (5.9 g, 76.541 mmol, 15.02 equiv) in MeOH (15 mL) was added NaBH₃CN (1.6 g, 25.461 mmol, 4.99 equiv) in portions at 0° C. under nitrogen atmosphere. The resulting mixture was stirred overnight at 80° C. under nitrogen atmosphere. The resulting mixture was diluted with water (100 mL) and extracted with EtOAc (3×100 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (1:2) to afford 7-fluoro-3-methylisoindolin-1-one (500 mg, 59%) as an off-white solid. LC-MS (ES, m/z)=165.9 [M+1]⁺.

Step 3. Preparation of 4-fluoro-1-methylisoindoline. To a stirred solution of 7-fluoro-3-methylisoindolin-1-one (500.0 mg, 3.027 mmol, 1 equiv) in THF (5 mL) was added BH₃-THF (15 mL, 15.135 mmol, 5 equiv) dropwise at 0° C. under nitrogen atmosphere. The resulting mixture was stirred overnight at 80° C. under nitrogen atmosphere. The mixture was allowed to cool down to room temperature. The reaction was quenched with Water/Ice at 0° C. The mixture was acidified to pH<7 with 1M HCl (aq.). The resulting mixture was concentrated under reduced pressure. The residue was purified by reversed-phase flash chromatography to afford 4-fluoro-1-methylisoindoline (300 mg, 66%) as an off-white solid. LC-MS (ES, m/z)=152.1 [M+1]⁺.

Step 4. Preparation of 3-(5-(4-fluoro-1-methylisoindoline-2-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (100.0 mg, 0.347 mmol, 1.00 equiv), 4-fluoro-1-methylisoindoline (62.9 mg, 0.416 mmol, 1.20 equiv) in DMF (2 mL) and DIEA (224.2 mg, 1.735 mmol, 5.00 equiv) was added HATU (197.9 mg, 0.520 mmol, 1.50 equiv) at room temperature under nitrogen atmosphere. The resulting mixture was stirred for 3 h at room temperature under nitrogen atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(4-fluoro-1-methylisoindoline-2-carbonyl)-1-oxoisoindolin-2-yl)piperidine-2,6-dione (17.5 mg, 12%) as an off-white solid. LC-MS (ES, m/z)=420.1 [M−1]⁻. ¹H NMR (400 MHz, DMSO-d₆) δ 11.01 (s, 1H), 7.83 (dd, J=7.8, 3.4 Hz, 2H), 7.76-7.69 (m, 1H), 7.40 (td, J=7.8, 5.2 Hz, 1H), 7.28-7.08 (m, 2H), 5.51 (d, J=6.7 Hz, 1H), 5.16 (dd, J=13.2, 5.0 Hz, 1H), 5.01 (t, J=16.1 Hz, 1H), 4.62-4.34 (m, 3H), 2.93 (ddd, J=17.2, 13.6, 5.4 Hz, 1H), 2.66-2.57 (m, 1H), 2.48-2.35 (m, 1H), 2.04 (dt, J=7.8, 4.6 Hz, 1H), 1.57 (d, J=6.4 Hz, 2H), 1.07 (d, J=6.4 Hz, 1H).

### Example 137

### Synthesis of 3-(6-(4-fluoroisoindoline-2-carbonyl)benzo[d]oxazol-2-yl)piperidine-2,6-dione

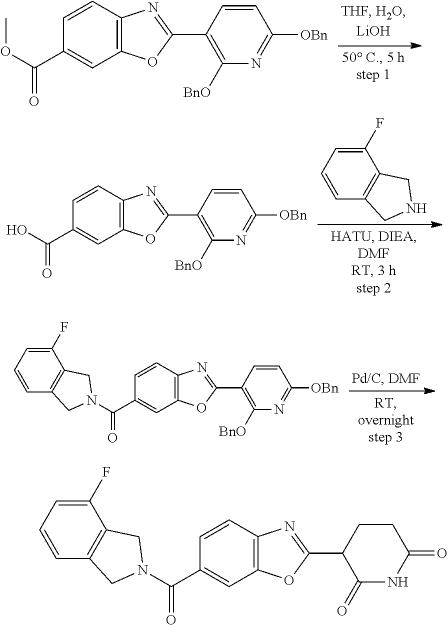

Step 1. Preparation of 2-(2,6-bis(benzyloxy)pyridin-3-yl)benzo[d]oxazole-6-carboxylic acid. A solution of methyl 2-[2,6-bis(benzyloxy)pyridin-3-yl]-1,3-benzoxazole-6-carboxylate (110.0 mg, 0.236 mmol, 1 equiv) and LiOH (113.0 mg, 4.720 mmol, 20 equiv) in THF (3 mL) and H₂O (1 mL) was stirred for 5 h at 50° C. under nitrogen atmosphere. The resulting mixture was concentrated under reduced pressure. The residue was diluted with water and acidified to pH 4 with 1 N HCl. The resulting mixture was extracted with CH₂Cl₂ (3×30 mL). The combined organic layers were washed with brine (2×50 mL), dried over anhydrous Na₂SO₄. After filtration, the filtrate was concentrated under reduced pressure. The crude product was used in the next step directly without further purification. LC-MS (ES, m/z)=453.5 [M+1]⁺.

Step 2. Preparation of 2-(2,6-bis(benzyloxy)pyridin-3-yl)benzo[d]oxazole-6-carboxylic acid. A solution of 2-(2,6-bis(benzyloxy)pyridin-3-yl)benzo[d]oxazole-6-carboxylic acid (99.0 mg, 0.218 mmol, 2 equiv) in DMF (2 mL) was treated with 4-fluoro-2,3-dihydro-1H-isoindole (15.0 mg, 0.109 mmol, 1.00 equiv) and DIEA (56.5 mg, 0.436 mmol, 4 equiv) at room temperature under nitrogen atmosphere followed by the addition of HATU (62.4 mg, 0.164 mmol, 1.5 equiv) at room temperature. The resulting mixture was stirred for 3 h at room temperature under nitrogen atmosphere. The resulting mixture was diluted with water (30 mL). The resulting mixture was extracted with EtOAc (3×30 mL). The combined organic layers were washed with water (3×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)benzo[d]oxazole-6-carboxylic acid (60 mg, 96%) as an off-white solid. LC-MS (ES, m/z)=572.0 [M+1]⁺.

Step 3. Preparation of 3-(6-(4-fluoroisoindoline-2-carbonyl)benzo[d]oxazol-2-yl)piperidine-2,6-dione. To a stirred solution of 2-(2,6-bis(benzyloxy)pyridin-3-yl)benzo[d]oxazole-6-carboxylic acid (90.0 mg, 0.157 mmol, 1 equiv) in DMF (2 mL) was added Palladium 10% on Carbon (wetted with ca. 55% Water) (90.5 mg) at room temperature under nitrogen atmosphere. The resulting mixture was stirred overnight at room temperature under hydrogen atmosphere. The resulting mixture was filtered, the filter cake was washed with EtOAc (3×5 mL). The filtrate was concentrated under reduced pressure. The residue was purified by reversed-phase flash chromatography to afford 3-(6-(4-fluoroisoindoline-2-carbonyl)benzo[d]oxazol-2-yl)piperidine-2,6-dione (8.0 mg, 12%) as a white solid. LC-MS (ES, m/z)=394.0 [M+1]⁺. ¹H NMR (400 MHz, DMSO-d₆) δ 11.16 (s, 1H), 8.10-8.05 (m, 1H), 7.85 (d, J=8.2 Hz, 1H), 7.64 (ddd, J=8.2, 5.2, 1.5 Hz, 1H), 7.40-7.30 (m, 1H), 7.29-7.08 (m, 2H), 5.05-4.79 (m, 4H), 4.57 (dd, J=11.3, 5.0 Hz, 1H), 2.77-2.62 (m, 2H), 2.46-2.28 (m, 2H).

### Example 138

### Synthesis of 3-(5-(2,2-difluoro-7-azaspiro[3.5]nonane-7-carbonyl)-1-oxoisoindolin-2-yl)piperidine-2,6-dione

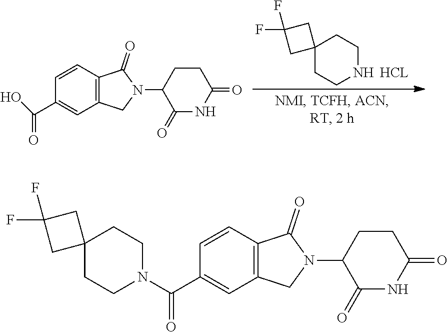 [SMILES: O=C1CCC(N2Cc3cc(C(=O)O)ccc3C2=O)C(=O)N1 ; FC1(F)CC2(CCNCC2)C1 ; O=C1CCC(N2Cc3cc(C(=O)N4CCC5(CC4)CC(F)(F)C5)ccc3C2=O)C(=O)N1]

Step 1. Preparation of 3-(5-(2,2-difluoro-7-azaspiro[3.5]nonane-7-carbonyl)-1-oxoisoindolin-2-yl)piperidine-2,6-dione. To a stirred solution of 2,2-difluoro-7-azaspiro[3.5]nonane (50.0 mg, 0.310 mmol, 1 equiv), 2-(2,6-dioxopiperidin-3-yl)-1-oxo-3H-isoindole-5-carboxylic acid (107.3 mg, 0.372 mmol, 1.2 equiv) and NMI (101.9 mg, 1.240 mmol, 4 equiv) in ACN (1 mL) was added TCFH (130.6 mg, 0.465 mmol, 1.5 equiv) at room temperature under nitrogen atmosphere. The resulting mixture was stirred for 2 h at room temperature under nitrogen atmosphere. The resulting mixture was diluted with water (50 mL) and extracted with EtOAc (30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(2,2-difluoro-7-azaspiro[3.5]nonane-7-carbonyl)-1-oxoisoindolin-2-yl)piperidine-2,6-dione (57 mg, 42%) as an off-white solid. LC-MS (ES, m/z)=432.3 [M+1]⁺. ¹H NMR (400 MHz, DMSO-d₆) δ 11.01 (s, 1H), 7.78 (d, J=7.8 Hz, 1H), 7.62 (s, 1H), 7.53-7.45 (m, 1H), 5.14 (dd, J=13.3, 5.0 Hz, 1H), 4.51 (d, J=17.5 Hz, 1H), 4.37 (d, J=17.7 Hz, 1H), 3.60 (s, 2H), 3.23 (s, 2H), 2.95-2.85 (m, 1H), 2.63 (s, 2H), 2.41 (d, J=13.1 Hz, 4H), 2.01 (t, J=6.0 Hz, 1H), 1.63 (d, J=29.0 Hz, 4H).

### Examples 139 and 140

### Synthesis of 3-(5-((R)-7-fluoro-1-methylisoindoline-2-carbonyl)-1-oxoisoindolin-2-yl)piperidine-2,6-dione and 3-(5-((S)-7-fluoro-1-methylisoindoline-2-carbonyl)-1-oxoisoindolin-2-yl)piperidine-2,6-dione

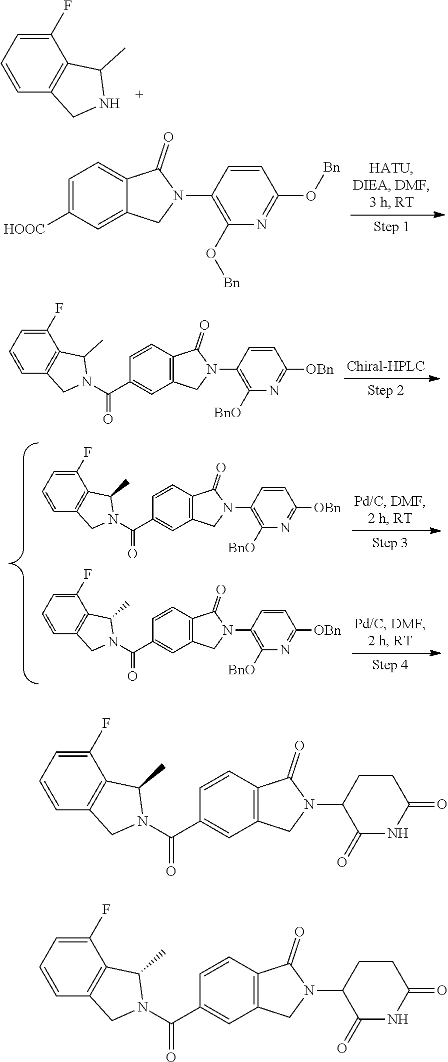

Step 1. Preparation of 2-(2,6-bis(benzyloxy)pyridin-3-yl)-5-(7-fluoro-1-methylisoindoline-2-carbonyl)isoindolin-1-one. A solution of 7-fluoro-1-methyl-2,3-dihydro-1H-isoindole (518.4 mg, 3.428 mmol, 4 equiv), DIEA (554.12 mg, 4.285 mmol, 5 equiv) and 2-[2,6-bis(benzyloxy)pyridin-3-yl]-1-oxo-3H-isoindole-5-carboxylic acid (400 mg, 0.857 mmol, 1.00 equiv) in DMF (10 mL) was stirred for 3 min at room temperature. To the above mixture was added HATU (489.0 mg, 1.285 mmol, 1.5 equiv) in portions over 3 min at room temperature. The resulting mixture was diluted with water (100 mL) and extracted with EtOAc (3×50 mL). The combined organic layers were washed with brine (2×100 mL), dried over anhydrous Na₂SO₄. After filtration, the filtrate was concentrated under reduced pressure. The resulting mixture was stirred for an additional 3 h at room temperature. The residue was purified by reversed-phase flash chromatography to afford 2-(2,6-bis(benzyloxy)pyridin-3-yl)-5-(7-fluoro-1-methylisoindoline-2-carbonyl)isoindolin-1-one (500 mg, 97%) as a light-yellow oil. LC-MS (ES, m/z)=600.30 [M+1]⁺.

Step 2. Preparation of (R)-2-(2,6-bis(benzyloxy)pyridin-3-yl)-5-(7-fluoro-1-methylisoindoline-2-carbonyl)isoindolin-1-one and (S)-2-(2,6-bis(benzyloxy)pyridin-3-yl)-5-(7-fluoro-1-methylisoindoline-2-carbonyl)isoindolin-1-one. The crude 2-(2,6-bis(benzyloxy)pyridin-3-yl)-5-(7-fluoro-1-methylisoindoline-2-carbonyl)isoindolin-1-one (400.0 mg) was purified by Prep-Chiral-HPLC with the following conditions (Column: CHIRAL ART Cellulose-SZ, 3*25 cm, 5 μm; Mobile Phase A: Hex (10 mM NH₃-MeOH), Mobile Phase B: EtOH-HPLC; Flow rate: 40 mL/min; Gradient: isocratic 50; Wave Length: 220/230 nm; RT1 (min): 18.4; RT2 (min): 29.4; Sample Solvent: EtOH:DCM=2:1; Injection Volume: 1 mL; Number Of Runs: 5) to afford (R)-2-(2,6-bis(benzyloxy)pyridin-3-yl)-5-(7-fluoro-1-methylisoindoline-2-carbonyl)isoindolin-1-one (200 mg, 50%, RT: 1.84 min) as an off-white oil and (S)-2-(2,6-bis(benzyloxy)pyridin-3-yl)-5-(7-fluoro-1-methylisoindoline-2-carbonyl)isoindolin-1-one (190 mg, 48%, RT: 2.826 min) as an off-white oil. LC-MS (ES, m/z)=600.30 [M+1]⁺.

Step 3. Preparation of 3-(5-((R)-7-fluoro-1-methylisoindoline-2-carbonyl)-1-oxoisoindolin-2-yl)piperidine-2,6-dione. A solution of (R)-2-(2,6-bis(benzyloxy)pyridin-3-yl)-5-(7-fluoro-1-methylisoindoline-2-carbonyl)isoindolin-1-one (100.0 mg, 0.167 mmol, 1 equiv) and palladium 10% on Carbon (wetted with ca. 55% Water) (177.5 mg, 0.167 mmol, 1 equiv) in DMF (3 mL) was stirred for 2 h at room temperature under hydrogen atmosphere. The resulting mixture was filtered, the filter cake was washed with DCM (3×10 mL). The filtrate was concentrated under reduced pressure. The residue was purified by reversed-phase flash chromatography to afford 3-(5-((R)-7-fluoro-1-methylisoindoline-2-carbonyl)-1-oxoisoindolin-2-yl)piperidine-2,6-dione (35 mg, 49%) as a light pink solid. LC-MS (ES, m/z)=422.20 [M+1]⁺. ¹H NMR (400 MHz, DMSO-d₆) δ 11.02 (s, 1H), 7.84 (dd, J=9.3, 6.0 Hz, 2H), 7.72 (ddd, J=7.9, 3.5, 1.4 Hz, 1H), 7.45-7.25 (m, 1H), 7.18-7.00 (m, 2H), 5.62 (q, J=6.4 Hz, 1H), 5.22-5.11 (m, 1H), 5.08-4.82 (m, 1H), 4.62-4.34 (m, 3H), 2.93 (ddd, J=17.2, 13.6, 5.4 Hz, 1H), 2.68-2.55 (m, 1H), 2.42 (td, J=13.2, 4.4 Hz, 1H), 2.09-1.96 (m, 1H), 1.59 (d, J=6.4 Hz, 2H), 1.10 (dd, J=6.3, 3.3 Hz, 1H).

Step 4. Preparation of 3-(5-((S)-7-fluoro-1-methylisoindoline-2-carbonyl)-1-oxoisoindolin-2-yl)piperidine-2,6-dione. A solution of (S)-2-(2,6-bis(benzyloxy)pyridin-3-yl)-5-(7-fluoro-1-methylisoindoline-2-carbonyl)isoindolin-1-one (100.0 mg, 0.167 mmol, 1 equiv) and palladium 10% on Carbon (wetted with ca. 55% Water) (177.5 mg, 0.167 mmol, 1 equiv) in DMF (2.5 mL) was stirred for 2 h at room temperature under hydrogen atmosphere. The resulting mixture was filtered, the filter cake was washed with DCM (3×10 mL). The filtrate was concentrated under reduced pressure. The residue was purified by reversed-phase flash chromatography to afford 3-(5-((S)-7-fluoro-1-methylisoindoline-2-carbonyl)-1-oxoisoindolin-2-yl)piperidine-2,6-dione (30 mg, 43%) as an off-white solid. LC-MS (ES, m/z)=422.20 [M+1]⁺. ¹H NMR (400 MHz, DMSO-d₆) δ 11.02 (s, 1H), 7.89-7.77 (m, 2H), 7.75-7.67 (m, 1H), 7.35 (tp, J=20.7, 7.5 Hz, 1H), 7.12 (dt, J=14.2, 8.0 Hz, 2H), 5.62 (q, J=6.4 Hz, 1H), 5.16 (dd, J=13.4, 5.0 Hz, 1H), 5.07-4.84 (m, 1H), 4.64-4.32 (m, 3H), 2.93 (ddd, J=17.9, 13.4, 5.3 Hz, 1H), 2.70-2.55 (m, 1H), 2.42 (td, J=13.0, 4.3 Hz, 1H), 2.11-1.96 (m, 1H), 1.59 (d, J=6.4 Hz, 2H), 1.10 (dd, J=6.5, 3.3 Hz, 1H).

### Examples 141 and 142

### Synthesis of 3-(5-((S)-4-fluoro-1-methylisoindoline-2-carbonyl)-1-oxoisoindolin-2-yl)piperidine-2,6-dione and 3-(5-((R)-4-fluoro-1-methylisoindoline-2-carbonyl)-1-oxoisoindolin-2-yl)piperidine-2,6-dione

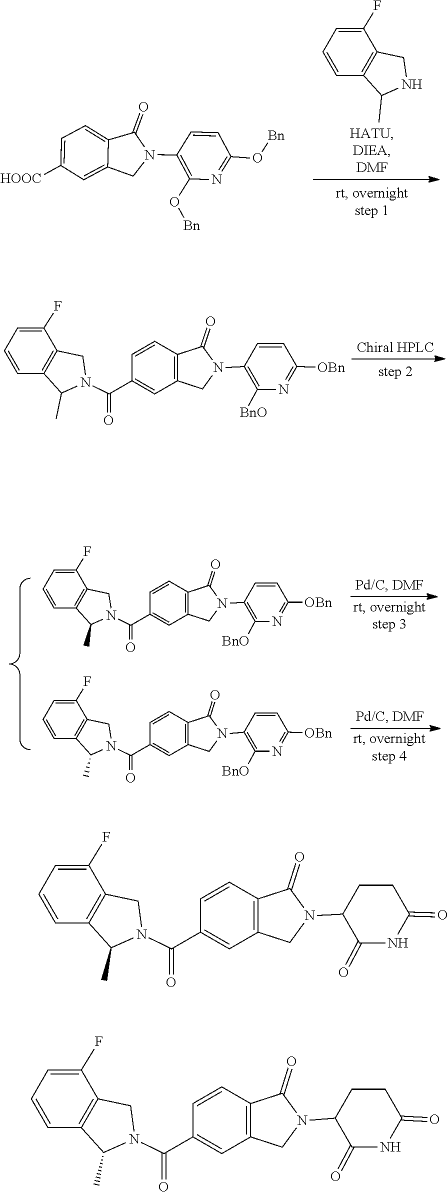

Step 1. Preparation of 2-(2,6-bis(benzyloxy)pyridin-3-yl)-5-(4-fluoro-1-methylisoindoline-2-carbonyl)isoindolin-1-one. To a stirred mixture of 4-fluoro-1-methyl-2,3-dihydro-1H-isoindole (194.5 mg, 1.285 mmol, 1.5 equiv) and 2-[2,6-bis(benzyloxy)pyridin-3-yl]-1-oxo-3H-isoindole-5-carboxylic acid (400.0 mg, 0.857 mmol, 1.00 equiv) in DMF (6 mL) was added DIEA (332.5 mg, 2.571 mmol, 3 equiv) at room temperature. To the above mixture was added HATU (489.1 mg, 1.285 mmol, 1.5 equiv) at room temperature. The resulting mixture was stirred for additional overnight at room temperature. The resulting mixture was diluted with water (50 mL) and extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(4-fluoro-1-methylisoindoline-2-carbonyl)isoindolin-1-one (450 mg, 88%) as a brown yellow solid. LC-MS (ES, m/z)=600.3 [M+1]⁺.

Step 2. Preparation of (S)-2-(2,6-bis(benzyloxy)pyridin-3-yl)-5-(4-fluoro-1-methylisoindoline-2-carbonyl)isoindolin-1-one and (R)-2-(2,6-bis(benzyloxy)pyridin-3-yl)-5-(4-fluoro-1-methylisoindoline-2-carbonyl)isoindolin-1-one. The product 2-(2,6-bis(benzyloxy)pyridin-3-yl)-5-(4-fluoro-1-methylisoindoline-2-carbonyl)isoindolin-1-one (400.0 mg, 0.667 mmol, 1 equiv) was purified by Chiral HPLC with the following conditions: Column: CHIRAL ART Cellulose-SZ, 3*25 cm, 5 μm; Mobile Phase A: Hex (10 mM NH₃-MeOH), Mobile Phase B: EtOH-HPLC; Flow rate: 40 mL/min; Gradient: isocratic 50; Wave Length: 205/210 nm; RT1 (min): 17.6; RT2 (min): 29.8; Sample Solvent: ETOH:DCM=9:1; Injection Volume: 1.5 mL; Number Of Runs: 6, to afford (S)-2-(2,6-bis(benzyloxy)pyridin-3-yl)-5-(4-fluoro-1-methylisoindoline-2-carbonyl)isoindolin-1-one (180 mg, 45) as a yellow solid and (R)-2-(2,6-bis(benzyloxy)pyridin-3-yl)-5-(4-fluoro-1-methylisoindoline-2-carbonyl)isoindolin-1-one (185 mg, 46%) as a yellow solid. LC-MS (ES, m/z)=600.3 [M+1]⁺.

Step 3. Preparation of 3-(5-((S)-4-fluoro-1-methylisoindoline-2-carbonyl)-1-oxoisoindolin-2-yl)piperidine-2,6-dione. To a solution of (S)-2-(2,6-bis(benzyloxy)pyridin-3-yl)-5-(4-fluoro-1-methylisoindoline-2-carbonyl)isoindolin-1-one (150.0 mg, 0.250 mmol, 1 equiv) in DMF (3 mL) was added Palladium 10% on Carbon (wetted with ca. 55% Water) (75.0 mg) under nitrogen atmosphere in a 10 mL round-bottom flask. The mixture was hydrogenated at room temperature for 2 h under hydrogen atmosphere using a hydrogen balloon, filtered through a Celite pad and concentrated under reduced pressure. The residue was purified by reversed-phase flash chromatography to afford 3-(5-((S)-4-fluoro-1-methylisoindoline-2-carbonyl)-1-oxoisoindolin-2-yl)piperidine-2,6-dione (83 mg, 79%) as an off-white solid. LC-MS (ES, m/z)=422.0 [M+1]⁺. ¹H NMR (400 MHz, DMSO-d₆) δ 11.03 (s, 1H), 7.84 (dd, J=7.4, 2.8 Hz, 2H), 7.73 (d, J=7.8 Hz, 1H), 7.39 (t, J=6.6 Hz, 1H), 7.26 (d, J=7.5 Hz, 1H), 7.18-7.08 (m, 1H), 5.52 (d, J=6.7 Hz, 1H), 5.17 (dd, J=13.3, 5.0 Hz, 1H), 5.01 (t, J=14.8 Hz, 1H), 4.64-4.33 (m, 3H), 2.94 (ddd, J=18.0, 13.4, 5.3 Hz, 1H), 2.62 (d, J=17.0 Hz, 1H), 2.47-2.32 (m, 1H), 2.09-1.99 (m, 1H), 1.57 (d, J=6.4 Hz, 2H), 1.07 (d, J=6.4 Hz, 1H).

Step 4. Preparation of 3-(5-((R)-4-fluoro-1-methylisoindoline-2-carbonyl)-1-oxoisoindolin-2-yl)piperidine-2,6-dione. To a solution of (R)-2-(2,6-bis(benzyloxy)pyridin-3-yl)-5-(4-fluoro-1-methylisoindoline-2-carbonyl)isoindolin-1-one (150.0 mg, 0.250 mmol, 1 equiv) in DMF (3 mL) was added Palladium 10% on Carbon (wetted with ca. 55% Water) (75.0 mg) under nitrogen atmosphere in a 10 mL round-bottom flask. The mixture was hydrogenated at room temperature for 2 h under hydrogen atmosphere using a hydrogen balloon, filtered through a Celite pad and concentrated under reduced pressure. The residue was purified by reversed-phase flash chromatography to afford 3-(5-((R)-4-fluoro-1-methylisoindoline-2-carbonyl)-1-oxoisoindolin-2-yl)piperidine-2,6-dione (94 mg, 90%) as an off-white solid. LC-MS (ES, m/z)=422.1 [M+1]⁺. ¹H NMR (400 MHz, DMSO-d₆) δ 11.02 (s, 1H), 7.87-7.80 (m, 2H), 7.72 (d, J=8.0 Hz, 1H), 7.40 (td, J=7.9, 5.3 Hz, 1H), 7.28-7.08 (m, 2H), 5.54-5.27 (m, 1H), 5.16 (dd, J=13.3, 5.0 Hz, 1H), 5.06-4.85 (m, 1H), 4.62-4.34 (m, 3H), 2.93 (ddd, J=18.0, 13.6, 5.3 Hz, 1H), 2.66-2.57 (m, 1H), 2.42 (tt, J=13.1, 6.5 Hz, 1H), 2.03 (dd, J=12.1, 6.4 Hz, 1H), 1.57 (d, J=6.4 Hz, 2H), 1.07 (d, J=6.3 Hz, 1H).

### Example 143

### Synthesis of (6-(2,4-dioxotetrahydropyrimidin-1(2H)-yl)naphthalen-2-yl)methyl(2-fluoro-5-(trifluoromethoxy)phenyl)carbamate

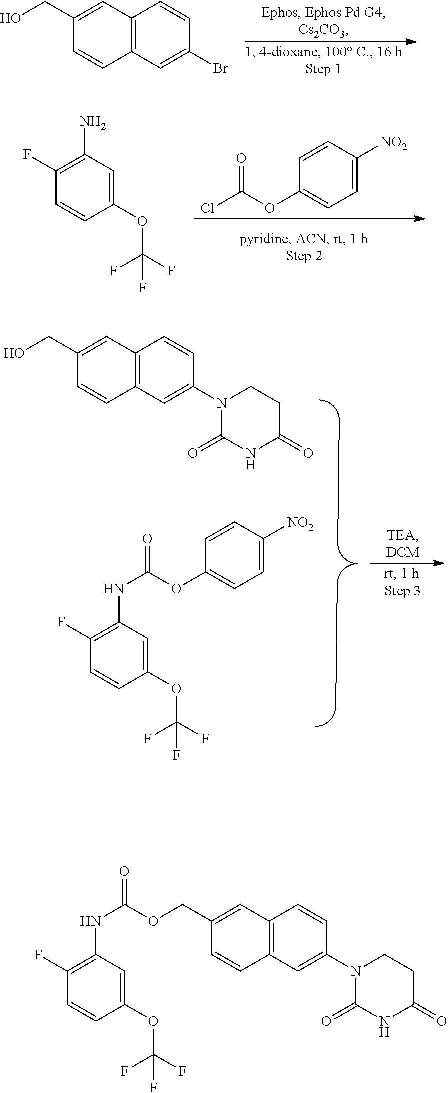 [SMILES: OCc1ccc2cc(Br)ccc2c1 ; Nc1cc(OC(F)(F)F)ccc1F ; O=C(Cl)Oc1ccc([N+](=O)[O-])cc1 ; O=C1CCN(c2ccc3cc(CO)ccc3c2)C(=O)N1 ; O=C(Nc1cc(OC(F)(F)F)ccc1F)Oc1ccc([N+](=O)[O-])cc1 ; O=C1CCN(c2ccc3cc(COC(=O)Nc4cc(OC(F)(F)F)ccc4F)ccc3c2)C(=O)N1]

Step 1. Preparation of 1-(6-(hydroxymethyl)naphthalen-2-yl)dihydropyrimidine-2,4(1H,3H)-dione. To a stirred mixture of (6-bromonaphthalen-2-yl)methanol (155.0 mg, 0.655 mmol, 1 equiv) and 5,6-dihydrouracil (149.4 mg, 1.310 mmol, 2 equiv) in 1,4-dioxane (6 mL) were added Cs₂CO₃ (426.6 mg, 1.310 mmol, 2 equiv), Ephos (35.0 mg, 0.066 mmol, 0.1 equiv) and Ephos Pd G₄ (60.1 mg, 0.066 mmol, 0.1 equiv) at room temperature under nitrogen atmosphere. The resulting mixture was stirred for 16 h at 80° C. under nitrogen atmosphere. The resulting mixture was diluted with water (50 mL) and extracted with EtOAc (3×30 mL). The combined organic layers were washed with brine (2×50 mL), dried over anhydrous MgSO₄. After filtration, the filtrate was concentrated under reduced pressure. The residue was purified by reversed-phase flash chromatography to afford 1-(6-(hydroxymethyl)naphthalen-2-yl)dihydropyrimidine-2,4(1H,3H)-dione (120 mg, 37%) as an off-white solid. LC-MS (ES, m/z)=271.15 [M+1]⁺.

Step 2. Preparation of 4-nitrophenyl (2-fluoro-5-(trifluoromethoxy)phenyl)carbamate. To a stirred solution of 2-fluoro-5-(trifluoromethoxy)aniline (200.0 mg, 1.025 mmol, 1 equiv) and pyridine (324.3 mg, 4.100 mmol, 4 equiv) in ACN (4 mL) were added 4-nitrophenyl carbonochloridate (371.9 mg, 1.845 mmol, 1.8 equiv) in portions at 0° C. under nitrogen atmosphere. The resulting mixture was stirred for 1 h at room temperature under nitrogen atmosphere. The resulting mixture was diluted with water (50 mL) and extracted with EtOAc (30 mL). The combined organic layers were washed with 2×30 mL of brine and dried over anhydrous Na₂SO₄. After filtration, the filtrate was concentrated under reduced pressure. The residue was purified by reversed-phase flash chromatography to afford 4-nitrophenyl (2-fluoro-5-(trifluoromethoxy)phenyl)carbamate (150 mg, 41%) as an off-white solid. LC-MS (ES, m/z)=361.25 [M+1]⁺.

Step 3. Preparation of (6-(2,4-dioxotetrahydropyrimidin-1(2H)-yl)naphthalen-2-yl)methyl (2-fluoro-5-(trifluoromethoxy)phenyl)carbamate. To a stirred mixture of 1-(6-(hydroxymethyl)naphthalen-2-yl)dihydropyrimidine-2,4(1H,3H)-dione (100.0 mg, 0.370 mmol, 1 equiv) and TEA (112.3 mg, 1.110 mmol, 3 equiv) in DCM (2 mL) was added 4-nitrophenyl (2-fluoro-5-(trifluoromethoxy)phenyl)carbamate (146.6 mg, 0.407 mmol, 1.1 equiv) in portions at 0° C. under nitrogen atmosphere. The resulting mixture was stirred for 1 h at room temperature under nitrogen atmosphere. The resulting mixture was diluted with water (30 mL) and extracted with DCM (20 mL). The combined organic layers were washed with brine (2×20 mL), dried over anhydrous MgSO₄. After filtration, the filtrate was concentrated under reduced pressure. The residue was purified by reversed-phase flash chromatography to afford (6-(2,4-dioxotetrahydropyrimidin-1(2H)-yl)naphthalen-2-yl)methyl (2-fluoro-5-(trifluoromethoxy)phenyl)carbamate (50 mg, 27%) as an off-white solid. LC-MS (ES, m/z)=490.15 [M−1]⁻. ¹H NMR (400 MHz, DMSO-d₆) δ 10.46 (s, 1H), 9.99-9.78 (m, 1H), 7.99-7.90 (m, 3H), 7.85 (dd, J=6.9, 2.6 Hz, 2H), 7.58 (dt, J=8.8, 2.1 Hz, 2H), 7.39 (dd, J=10.4, 9.0 Hz, 1H), 7.14 (dt, J=9.0, 3.5 Hz, 1H), 5.36 (s, 2H), 3.93 (t, J=6.6 Hz, 2H), 2.77 (t, J=6.6 Hz, 2H).

### Example 144

### Synthesis of 3-(5-(4-chloro-5-fluoroisoindoline-2-carbonyl)-1-oxoisoindolin-2-yl)piperidine-2,6-dione

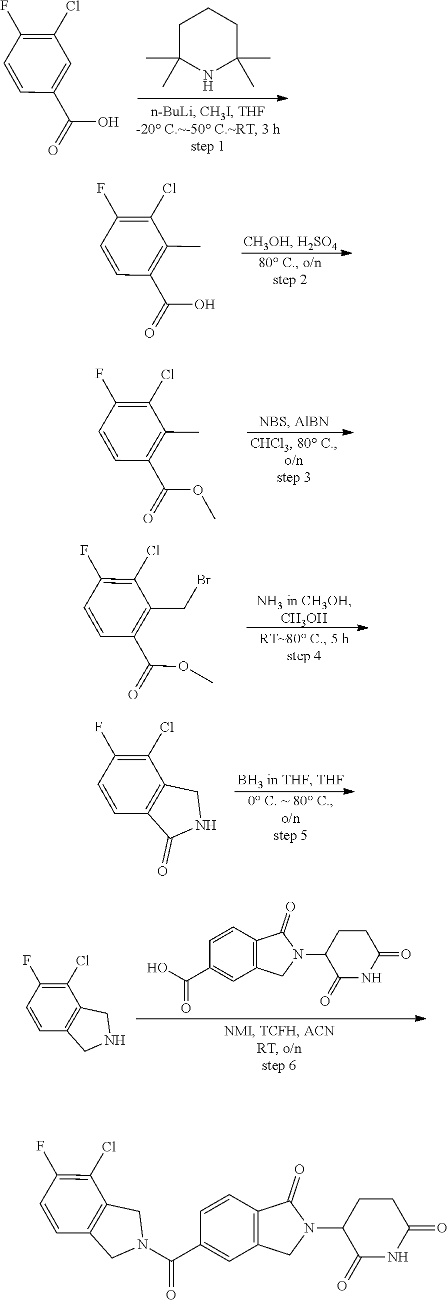

Step 1. Preparation of 3-chloro-4-fluoro-2-methylbenzoic acid. To a stirred solution of 2,2,6,6-tetramethylpiperidine (10.12 g, 71.608 mmol, 2.5 equiv) in THF (58 mL) was added n-BuLi (4.59 g, 71.608 mmol, 2.5 equiv) dropwise at −20° C. under nitrogen atmosphere. The resulting mixture was stirred for 1 h at −20° C. under nitrogen atmosphere. To the above mixture was added 3-chloro-4-fluorobenzoic acid (5 g, 28.643 mmol, 1 equiv) in THF (58 mL) dropwise over 10 min at −50° C. The resulting mixture was stirred for an additional 1 h at −50° C. To the above mixture was added methyl iodide (16.26 g, 114.572 mmol, 4 equiv) dropwise over 10 min at −50° C. The resulting mixture was stirred for an additional 1 h at room temperature. The reaction was quenched by the addition of sat. NH₄Cl (aq.) (50 mL) at 0° C. The resulting mixture was diluted with water (300 mL). The resulting mixture was extracted with EtOAc (3×300 mL). The combined organic layers were washed with brine (2×500 mL), dried over anhydrous Na₂SO₄. After filtration, the filtrate was concentrated under reduced pressure. The residue was purified by reversed-phase flash chromatography to afford 3-chloro-4-fluoro-2-methylbenzoic acid (5 g, 93%) as an off-white solid. LC-MS (ES, m/z)=187.0 [M−1]⁻.

Step 2. Preparation of methyl 3-chloro-4-fluoro-2-methylbenzoate. To a stirred solution of 3-chloro-4-fluoro-2-methylbenzoic acid (3 g, 15.908 mmol, 1 equiv) in CH₃OH (10 mL) was added H₂SO₄ (0.78 g, 7.954 mmol, 0.5 equiv) at room temperature under nitrogen atmosphere. The resulting mixture was stirred overnight at 80° C. under nitrogen atmosphere. The mixture was allowed to cool down to room temperature. The resulting mixture was diluted with water (200 mL). and extracted with EtOAc (3×200 mL). The combined organic layers were washed with brine (2×200 mL), dried over anhydrous Na₂SO₄. After filtration, the filtrate was concentrated under reduced pressure. The residue was purified by reversed-phase flash chromatography to afford methyl 3-chloro-4-fluoro-2-methylbenzoate (400 mg, 12%) as an off-white solid. LC-MS (ES, m/z)=no mass detected.

Step 3. Preparation of methyl 2-(bromomethyl)-3-chloro-4-fluorobenzoate. To a stirred solution of methyl 3-chloro-4-fluoro-2-methylbenzoate (700.0 mg, 3.455 mmol, 1 equiv) and NBS (922.3 mg, 5.183 mmol, 1.5 equiv) in chloroform (7 mL) was added AIBN (56.7 mg, 0.346 mmol, 0.1 equiv) at room temperature under nitrogen atmosphere. The resulting mixture was stirred overnight at 80° C. under nitrogen atmosphere. The mixture was allowed to cool down to room temperature. The resulting mixture was diluted with water (200 mL). The resulting mixture was extracted with EtOAc (3×100 mL). The combined organic layers were washed with brine (2×150 mL), dried over anhydrous Na₂SO₄. After filtration, the filtrate was concentrated under reduced pressure. The residue was purified by reversed-phase flash chromatography to afford methyl 2-(bromomethyl)-3-chloro-4-fluorobenzoate (600 mg, 62%) as a yellow solid. LC-MS (ES, m/z)=no mass detected.

Step 4. Preparation of 4-chloro-5-fluoroisoindolin-1-one. To a stirred solution of methyl 2-(bromomethyl)-3-chloro-4-fluorobenzoate (600.0 mg, 2.131 mmol, 1 equiv) in methanol (6 mL) was added NH₃ (g) in MeOH (145.2 mg, 8.524 mmol, 4 equiv) at room temperature under nitrogen atmosphere. The resulting mixture was stirred for 30 min at room temperature under nitrogen atmosphere. Then the resulting mixture was stirred for 5 h at 80° C. under nitrogen atmosphere. The mixture was allowed to cool down to room temperature. The resulting mixture was concentrated under reduced pressure. The residue was purified by reversed-phase flash chromatography to afford 4-chloro-5-fluoroisoindolin-1-one (300 mg, 76%) as a yellow solid. LC-MS (ES, m/z)=186.0 [M+1]⁺.

Step 5. Preparation of 4-chloro-5-fluoroisoindoline. To a stirred solution of 4-chloro-5-fluoroisoindolin-1-one (600.0 mg, 3.233 mmol, 1 equiv) in tetrahydrofuran (5 mL) was added BH₃-THF (16.17 mL, 16.165 mmol, 5 equiv) at 0° C. under nitrogen atmosphere. The resulting mixture was stirred overnight at 80° C. under nitrogen atmosphere. The mixture was allowed to cool down to room temperature. The reaction was quenched by the addition of MeOH (5 mL) at 0° C. The resulting mixture was concentrated under reduced pressure. The residue was diluted with water (100 mL). The resulting mixture was extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford 4-chloro-5-fluoroisoindoline (500 mg, 90%) as an off-white solid. LC-MS (ES, m/z)=172.0 [M+1]⁺.

Step 6. Preparation of 3-(5-(4-chloro-5-fluoroisoindoline-2-carbonyl)-1-oxoisoindolin-2-yl)piperidine-2,6-dione. To a stirred solution of 4-chloro-5-fluoroisoindoline (100.0 mg, 0.583 mmol, 1 equiv), 2-(2,6-dioxopiperidin-3-yl)-1-oxo-3H-isoindole-5-carboxylic acid (83.9 mg, 0.291 mmol, 0.5 equiv) and NMI (191.4 mg, 2.332 mmol, 4 equiv) in ACN (2 mL) was added TCFH (245.3 mg, 0.874 mmol, 1.5 equiv) at room temperature under nitrogen atmosphere. The resulting mixture was stirred overnight at room temperature under nitrogen atmosphere. The resulting mixture was diluted with water (50 mL). The resulting mixture was extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford 3-(5-(4-chloro-5-fluoroisoindoline-2-carbonyl)-1-oxoisoindolin-2-yl)piperidine-2,6-dione (16 mg, 6.29%) as an off-white solid. LC-MS (ES, m/z)=442.2 [M+1]⁺. ¹H NMR (400 MHz, DMSO-d₆) δ 11.03 (s, 1H), 7.89-7.81 (m, 2H), 7.76-7.69 (m, 1H), 7.49-7.22 (m, 2H), 5.16 (dd, J=13.3, 5.1 Hz, 1H), 4.94 (d, J=7.3 Hz, 2H), 4.81 (s, 2H), 4.61-4.33 (m, 2H), 3.02-2.84 (m, 1H), 2.62 (d, J 17.2 Hz, 1H), 2.49-2.34 (m, 1H), 2.04 (d, J=12.2 Hz, 1H).

### Example 145

### Synthesis of 3-(1-oxo-5-(6-(trifluoromethyl)-2-azaspiro[3.3]heptane-2-carbonyl)isoindolin-2-yl)piperidine-2,6-dione

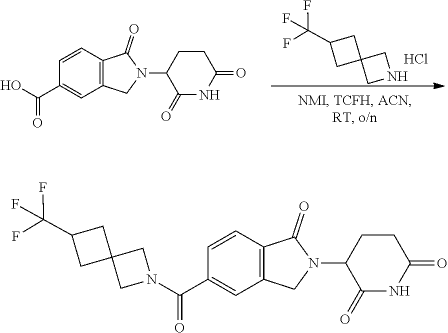 [SMILES: O=C1CCC(N2Cc3cc(C(=O)O)ccc3C2=O)C(=O)N1 ; FC(F)(F)C1CC2(CNC2)C1 ; Cl ; O=C1CCC(N2Cc3cc(C(=O)N4CC5(CC(C(F)(F)F)C5)C4)ccc3C2=O)C(=O)N1]

Step 1. Preparation of 3-(1-oxo-5-(6-(trifluoromethyl)-2-azaspiro[3.3]heptane-2-carbonyl)isoindolin-2-yl)piperidine-2,6-dione. To a stirred solution of 6-(trifluoromethyl)-2-azaspiro[3.3]heptane (50.0 mg, 0.303 mmol, 1 equiv), 2-(2,6-dioxopiperidin-3-yl)-1-oxo-3H-isoindole-5-carboxylic acid (87.3 mg, 0.303 mmol, 1 equiv) and NMI (99.4 mg, 1.212 mmol, 4 equiv) in ACN (2 mL) was added TCFH (127.4 mg, 0.455 mmol, 1.5 equiv) in at room temperature under nitrogen atmosphere. The resulting mixture was stirred for overnight at room temperature under nitrogen atmosphere. The resulting mixture was diluted with water (30 mL). The resulting mixture was extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(1-oxo-5-(6-(trifluoromethyl)-2-azaspiro[3.3]heptane-2-carbonyl)isoindolin-2-yl)piperidine-2,6-dione (38 mg, 29%) as an off-white solid. LC-MS (ES, m/z)=436.2 [M+1]⁺. ¹H NMR (400 MHz, DMSO-d₆) δ 11.02 (s, 1H), 7.85-7.70 (m, 3H), 5.14 (dd, J=13.2, 5.0 Hz, 1H), 4.55-4.29 (m, 4H), 4.09 (d, J=19.1 Hz, 2H), 3.13-2.85 (m, 2H), 2.61 (d, J=17.1 Hz, 1H), 2.47-2.36 (m, 2H), 2.30 (dd, J=13.6, 7.0 Hz, 2H), 2.06-1.97 (m, 1H).

### Example 146

### Synthesis of 3-(6-(1,2,3,4-tetrahydroisoquinoline-2-carbonyl)benzo[d]oxazol-2-yl)piperidine-2,6-dione

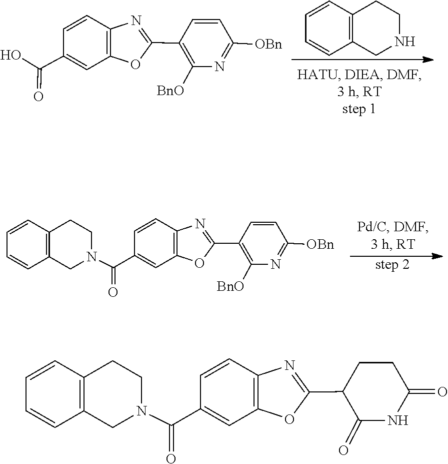 [SMILES: O=C(O)c1ccc2nc(-c3ccc(OCc4ccccc4)nc3OCc3ccccc3)oc2c1 ; c1ccc2c(c1)CCNC2 ; O=C(c1ccc2nc(-c3ccc(OCc4ccccc4)nc3OCc3ccccc3)oc2c1)N1CCc2ccccc2C1 ; O=C1CCC(c2nc3ccc(C(=O)N4CCc5ccccc5C4)cc3o2)C(=O)N1]

Step 1. Preparation of (2-(2,6-bis(benzyloxy)pyridin-3-yl)benzo[d]oxazol-6-yl)(3,4-dihydroisoquinolin-2(1H)-yl)methanone. A solution of 2-[2,6-bis(benzyloxy)pyridin-3-yl]-1,3-benzoxazole-6-carboxylic acid (100.1 mg, 0.221 mmol, 1.00 equiv), DIEA (85.7 mg, 0.663 mmol, 3 equiv) and tetrahydroisoquinoline (88.3 mg, 0.663 mmol, 3 equiv) in DMF (2 mL) was stirred for 3 min at room temperature. To the above mixture was added HATU (126.0 mg, 0.332 mmol, 1.5 equiv) at room temperature. The resulting mixture was stirred for an additional 3 h at room temperature. The resulting mixture was diluted with water (30 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(3,4-dihydroisoquinolin-2(1H)-yl)methanone (115 mg, 92%) as an off-white solid. LC-MS (ES, m/z)=568.35 [M+1]⁺.

Step 2. Preparation of 3-(6-(1,2,3,4-tetrahydroisoquinoline-2-carbonyl)benzo[d]oxazol-2-yl)piperidine-2,6-dione. To a stirred solution of (2-(2,6-bis(benzyloxy)pyridin-3-yl)benzo[d]oxazol-6-yl)(3,4-dihydroisoquinolin-2(1H)-yl)methanone (100.0 mg, 0.176 mmol, 1 equiv) in DMF (2 mL) was added Palladium 10% on Carbon (wetted with ca. 55% Water) (187.48 mg) at room temperature under nitrogen atmosphere. The resulting mixture was stirred overnight at room temperature under hydrogen atmosphere. The precipitated solids were collected by filtration and washed with DCM (3×10 mL). The filtrate was concentrated under reduced pressure. The residue was purified by reversed-phase flash chromatography to afford 3 (6-(1,2,3,4-tetrahydroisoquinoline-2-carbonyl)benzo[d]oxazol-2-yl)piperidine-2,6-dione (24 mg, 35%) as an off-white solid. LC-MS (ES, m/z)=390.2 [M+1]⁺. ¹H NMR (400 MHz, DMSO-d₆) δ 11.16 (s, 1H), 7.90 (s, 1H), 7.83 (d, J=8.1 Hz, 1H), 7.47 (d, J=8.1 Hz, 1H), 7.18 (s, 4H), 4.86-4.45 (m, 3H), 3.87 (s, 1H), 3.66-3.55 (m, 1H), 2.87 (s, 2H), 2.81-2.60 (m, 2H), 2.47-2.39 (m, 1H), 2.34 (dq, J=13.4, 5.0 Hz, 1H).

### Example 147

### Synthesis of 3-(5-(4,7-difluoroisoindoline-2-carbonyl)-1-oxoisoindolin-2-yl)piperidine-2,6-dione

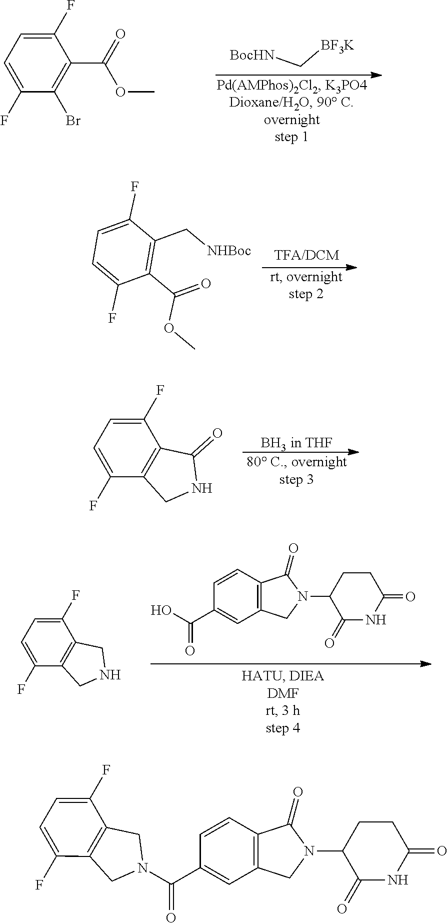

Step 1. Preparation of methyl 2-(((tert-butoxycarbonyl)amino)methyl)-3,6-difluorobenzoate. To a stirred mixture of methyl 2-bromo-3,6-difluorobenzoate (1.5 g, 5.975 mmol, 1 equiv) and tert-butyl ((trifluoro-γ⁴-boraneyl)methyl)carbamate, potassium salt (2.12 g, 8.962 mmol, 1.5 equiv) in dioxane (15 mL)/H₂O (3.8 mL) was added K₃PO₄ (2.54 g, 11.950 mmol, 2 equiv), Pd(AMPhos)₂Cl₂ (0.42 g, 0.597 mmol, 0.1 equiv) at room temperature under nitrogen atmosphere. The resulting mixture was stirred overnight at 90° C. under nitrogen atmosphere. The resulting mixture was diluted with water (100 mL) and extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford methyl 2-(((tert-butoxycarbonyl)amino)methyl)-3,6-difluorobenzoate (1 g, 56%) as a white oil. LC-MS (ES, m/z)=246.0 [M+1]⁺.

Step 2. Preparation of 4,7-difluoroisoindolin-1-one. To a stirred mixture of methyl 2-(((tert-butoxycarbonyl)amino)methyl)-3,6-difluorobenzoate (1 g, 3.319 mmol, 1 equiv) in DCM (10 mL) was added TFA (2 mL) dropwise at 0° C. The resulting mixture was stirred overnight at room temperature. The resulting mixture was concentrated under reduced pressure to afford 4,7-difluoroisoindolin-1-one (500 mg, crude) as a white oil. The residue was used in the next step directly without further purification.

Step 3. Preparation of 4,7-difluoroisoindoline. To a stirred mixture of 4,7-difluoroisoindolin-1-one (200.0 mg, 1.183 mmol, 1 equiv) in THF (4.00 mL) was added BH₃-THF (5.91 mL, 5.915 mmol, 5 equiv) at room temperature. The resulting mixture was stirred overnight at 80° C. under nitrogen atmosphere. The reaction was quenched by the addition of MeOH (2 mL) at 0° C. The resulting mixture was concentrated under reduced pressure. The residue was purified by reversed-phase flash chromatography to afford 4,7-difluoroisoindoline (150 mg, 82%) as a white solid. LC-MS (ES, m/z)=156.0 [M+1]⁺.

Step 4. Preparation of 3-(5-(4,7-difluoroisoindoline-2-carbonyl)-1-oxoisoindolin-2-yl)piperidine-2,6-dione. To a stirred mixture of 4,7-difluoro-2,3-dihydro-1H-isoindole (40.0 mg, 0.258 mmol, 1.5 equiv) and 2-(2,6-dioxopiperidin-3-yl)-1-oxo-3H-isoindole-5-carboxylic acid (49.5 mg, 0.172 mmol, 1 equiv) in ACN (1.5 mL) were added TCFH (72.3 mg, 0.258 mmol, 1.5 equiv) and NMI (56.4 mg, 0.688 mmol, 4 equiv) at room temperature. The resulting mixture was stirred for 3 h at room temperature. The resulting mixture was diluted with water (30 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(4,7-difluoroisoindoline-2-carbonyl)-1-oxoisoindolin-2-yl)piperidine-2,6-dione (12 mg, 16%) as a white solid. LC-MS (ES, m/z)=426.0 [M+1]⁺. ¹H NMR (400 MHz, DMSO-d₆) δ 11.01 (s, 1H), 7.94-7.80 (m, 2H), 7.74 (d, J=7.9 Hz, 1H), 7.33-7.10 (m, 2H), 5.16 (dd, J=13.3, 5.2 Hz, 1H), 4.93 (d, J=37.3 Hz, 4H), 4.55 (d, J=17.5 Hz, 2H), 2.94 (d, J=13.0 Hz, 1H), 2.62 (d, J=17.0 Hz, 1H), 2.04 (s, 1H), 1.24 (s, 1H).

### Example 148

### Synthesis of 3-(5-(6-chloro-4-fluoroisoindoline-2-carbonyl)-1-oxoisoindolin-2-yl)piperidine-2,6-dione

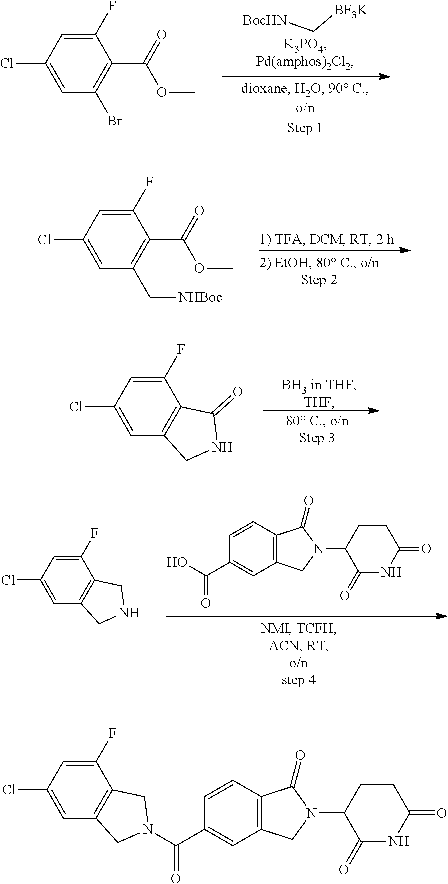

Step 1. Preparation of methyl 2-(((tert-butoxycarbonyl)amino)methyl)-4-chloro-6-fluorobenzoate. To a stirred solution of methyl 2-bromo-4-chloro-6-fluorobenzoate (5.0 g, 18.693 mmol, 1 equiv) and tert-butyl N-[(trifluoro-lambda4-boranyl)methyl]carbamate potassium (6.65 g, 28.040 mmol, 1.5 equiv) in dioxane (20 mL) and H₂O (5 mL) were added K₃PO₄ (7.94 g, 37.386 mmol, 2 equiv) and Pd(amphos)₂Cl₂ (2.65 g, 3.739 mmol, 0.2 equiv) at room temperature under nitrogen atmosphere. The resulting mixture was stirred overnight at 90° C. under nitrogen atmosphere. The mixture was allowed to cool down to room temperature. The resulting mixture was diluted with water (300 mL). The resulting mixture was extracted with EtOAc (3×200 mL). The combined organic layers were washed with brine (3×300 mL), dried over anhydrous Na₂SO₄. After filtration, the filtrate was concentrated under reduced pressure. The residue was purified by reversed-phase flash chromatography to afford methyl 2-(((tert-butoxycarbonyl)amino)methyl)-4-chloro-6-fluorobenzoate (600 mg, 10%) as a brown oil. LC-MS (ES, m/z)=262.0 [M-tBu+1]+.

Step 2. Preparation of 5-chloro-7-fluoroisoindolin-1-one. To a stirred solution of methyl 2-(((tert-butoxycarbonyl)amino)methyl)-4-chloro-6-fluorobenzoate (600.0 mg, 1.89 mmol, 1 equiv) in DCM (5 mL) was added TFA (1 mL) dropwise at 0° C. under nitrogen atmosphere. The resulting mixture was stirred for 2 h at room temperature under nitrogen atmosphere. The resulting mixture was concentrated under reduced pressure. To the above mixture was added ethyl alcohol (5 mL) at room temperature. The resulting mixture was stirred for additional overnight at 80° C. The mixture was allowed to cool down to room temperature. The resulting mixture was diluted with water (100 mL). The resulting mixture was extracted with EtOAc (3×100 mL). The combined organic layers were washed with brine (2×150 mL), dried over anhydrous Na₂SO₄. After filtration, the filtrate was concentrated under reduced pressure. The residue was purified by reversed-phase flash chromatography to afford 5-chloro-7-fluoroisoindolin-1-one (470 mg, 92%) as a brown oil. LC-MS (ES, m/z)=186.0 [M+1]⁺.

Step 3. Preparation of 6-chloro-4-fluoroisoindoline. To a stirred solution of 5-chloro-7-fluoroisoindolin-1-one (470.0 mg, 2.533 mmol, 1 equiv) in THF (2 mL) was added BH₃-THF (12.66 mL, 1M in THF) dropwise at 0° C. under nitrogen atmosphere. The resulting mixture was stirred overnight at 80° C. under nitrogen atmosphere. The mixture was allowed to cool down to room temperature. The reaction was quenched by the addition of MeOH (20 mL) at 0° C. The mixture was acidified to pH 3 with conc. HCl. The resulting mixture was concentrated under reduced pressure. The residue was purified by reversed-phase flash chromatography to afford 6-chloro-4-fluoroisoindoline (420 mg, 97%) as a yellow solid. LC-MS (ES, m/z)=172.0 [M+1]⁺.

Step 4 Preparation of 3-(5-(6-chloro-4-fluoroisoindoline-2-carbonyl)-1-oxoisoindolin-2-yl)piperidine-2,6-dione. To a stirred solution of 6-chloro-4-fluoroisoindoline (200.0 mg, 1.166 mmol, 1 equiv), 2-(2,6-dioxopiperidin-3-yl)-1-oxo-3H-isoindole-5-carboxylic acid (335.9 mg, 1.166 mmol, 1 equiv) and NMI (382.8 mg, 4.664 mmol, 4 equiv) in ACN (4 mL) was added TCFH (490.52 mg, 1.749 mmol, 1.5 equiv) in portions at room temperature under nitrogen atmosphere. The resulting mixture was stirred overnight at room temperature under nitrogen atmosphere. The resulting mixture was diluted with water (100 mL). The resulting mixture was extracted with EtOAc (3×100 mL). The combined organic layers were washed with brine (2×200 mL), dried over anhydrous Na₂SO₄. After filtration, the filtrate was concentrated under reduced pressure. The crude product (40.0 mg) was purified by Prep-HPLC to afford 3-(5-(6-chloro-4-fluoroisoindoline-2-carbonyl)-1-oxoisoindolin-2-yl)piperidine-2,6-dione (12.7 mg, 2.47%) as an off-white solid. LC-MS (ES, m/z)=442.2 [M+1]⁺. ¹H NMR (400 MHz, DMSO-d₆) δ 11.02 (s, 1H), 7.87-7.78 (m, 2H), 7.72 (dd, J=7.8, 5.3 Hz, 1H), 7.46-7.23 (m, 2H), 5.16 (dd, J=13.3, 5.1 Hz, 1H), 4.92 (d, J=7.5 Hz, 2H), 4.85-4.74 (m, 2H), 4.54 (dd, J=17.6, 4.9 Hz, 1H), 4.41 (dd, J=17.6, 4.4 Hz, 1H), 2.93 (ddd, J=18.0, 13.6, 5.4 Hz, 1H), 2.62 (ddd, J=17.3, 4.5, 2.3 Hz, 1H), 2.48-2.31 (m, 1H), 2.10-1.98 (m, 1H).

### Example 149

### Synthesis of 3-(5-(4-fluoro-5-methylisoindoline-2-carbonyl)-1-oxoisoindolin-2-yl)piperidine-2,6-dione

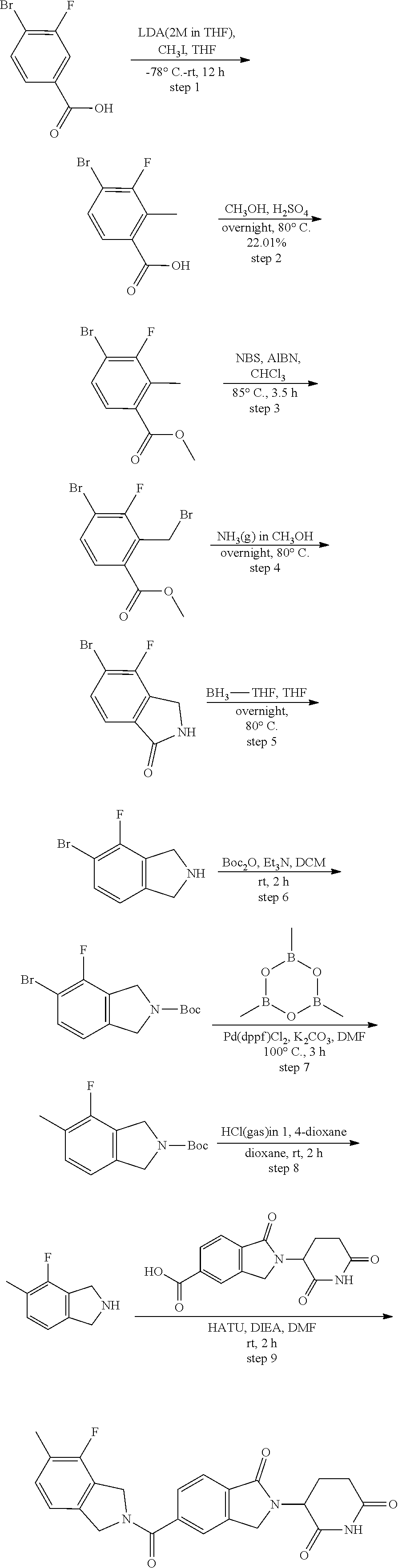

Step 1. Preparation of 4-bromo-3-fluoro-2-methylbenzoic acid. A solution of 4-bromo-3-fluorobenzoic acid (20 g, 91.320 mmol, 1 equiv) in THF (200 ml) was treated with LDA (192 mL, 2M in THF) for 1 h at −78° C. under nitrogen atmosphere followed by the addition of CH₃I (38.89 g, 273.960 mmol, 3 equiv) dropwise at −78° C. The resulting mixture was stirred for 12 h at room temperature under nitrogen atmosphere. The reaction was quenched with sat. NH₄Cl (aq.) at 0° C. The resulting mixture was diluted with water and extracted with EtOAc (3×250 mL). The combined organic layers were washed with brine (3×500 mL), dried over anhydrous Na₂SO₄. After filtration, the filtrate was concentrated under reduced pressure. The residue was purified by reversed-phase flash chromatography to afford 4-bromo-3-fluoro-2-methylbenzoic acid (30 g, crude) as a yellow solid. LC-MS (ES, m/z)=230.9 [M+1]⁺.

Step 2. Preparation of methyl 4-bromo-3-fluoro-2-methylbenzoate. A mixture of 4-bromo-3-fluoro-2-methylbenzoic acid (30 g, 128.735 mmol, 1 equiv) and H₂SO₄ (6.31 g, 64.368 mmol, 0.5 equiv) in MeOH (400 mL) was stirred overnight at 80° C. under nitrogen atmosphere. The resulting mixture was concentrated under reduced pressure. The resulting mixture was diluted with water (500 mL). The resulting mixture was extracted with EtOAc (3×500 mL). The combined organic layers were washed with brine (3×500 mL), dried over anhydrous Na₂SO₄. After filtration, the filtrate was concentrated under reduced pressure. The residue was purified by reversed-phase flash chromatography to afford methyl 4-bromo-3-fluoro-2-methylbenzoate (9 g, 22%) as a brown oil. LC-MS (ES, m/z)=no mass detected.

Step 3. Preparation of methyl 4-bromo-2-(bromomethyl)-3-fluorobenzoate. A mixture of methyl 3-fluoro-2-methylbenzoate (8.2 g, 48.761 mmol, 1 equiv), NBS (13.02 g, 73.142 mmol, 1.5 equiv) and AIBN (800.7 mg, 4.876 mmol, 0.1 equiv) in CHCl₃ (100 mL) was stirred for 3.5 h at 85° C. under nitrogen atmosphere. The resulting mixture was concentrated under reduced pressure. The resulting mixture was diluted with water (100 mL). The resulting mixture was extracted with EtOAc (3×200 mL). The combined organic layers were washed with brine (3×300 mL), dried over anhydrous Na₂SO₄. After filtration, the filtrate was concentrated under reduced pressure to afford methyl 4-bromo-2-(bromomethyl)-3-fluorobenzoate (12 g, 76%) as a Brown yellow oil. LC-MS (ES, m/z)=no mass detected.

Step 4. Preparation of 5-bromo-4-fluoroisoindolin-1-one. A mixture of methyl 4-bromo-2-(bromomethyl)-3-fluorobenzoate (10 g, 30.679 mmol, 1 equiv) in NH₃ (g) in MeOH (100 mL) was stirred overnight at 80° C. under nitrogen atmosphere. The resulting mixture was concentrated under reduced pressure. The residue was purified by silica gel column chromatography, eluted with PE/EA (1:1) to afford 5-bromo-4-fluoroisoindolin-1-one (5.5 g, 78%) as a yellow solid. LC-MS (ES, m/z)=229.95 [M+1]⁺.

Step 5. Preparation of 5-bromo-4-fluoroisoindoline. To a stirred mixture of 5-bromo-4-fluoroisoindolin-1-one (5 g, 21.736 mmol, 1 equiv) in THF (50 mL) was added BH₃-THF (500.22 mL, 1M) dropwise at 0° C. under nitrogen atmosphere. The resulting mixture was stirred overnight at 80° C. under nitrogen atmosphere. The reaction was quenched with Water/Ice at 0° C. The mixture was acidified to pH<7 with 1M HCl (aq.). The resulting mixture was concentrated under reduced pressure. The residue was purified by reversed-phase flash chromatography to afford 5-bromo-4-fluoroisoindoline (5 g, 100%) as a pink solid. LC-MS (ES, m/z)=216.0 [M+1]⁺.

Step 6. Preparation of tert-butyl 5-bromo-4-fluoroisoindoline-2-carboxylate. A mixture of 5-bromo-4-fluoroisoindoline (5 g, 23.142 mmol, 1 equiv), TEA (7.03 g, 69.426 mmol, 3 equiv) and Boc₂O (10.10 g, 46.284 mmol, 2 equiv) in DCM (80 mL) was stirred for 2 h at room temperature under nitrogen atmosphere. The resulting mixture was diluted with water (500 mL). The resulting mixture was extracted with DCM (3×200 mL). The combined organic layers were washed with brine (3×300 mL), dried over anhydrous Na₂SO₄. After filtration, the filtrate was concentrated under reduced pressure. The residue was purified by reversed-phase flash chromatography to afford tert-butyl 5-bromo-4-fluoroisoindoline-2-carboxylate (2.7 g, 37%) as a yellow solid. LC-MS (ES, m/z)=301.0 [M-t-Bu+ACN]⁺.

Step 7. Preparation of tert-butyl 4-fluoro-5-methylisoindoline-2-carboxylate. A mixture of tert-butyl 5-bromo-4-fluoroisoindoline-2-carboxylate (150.0 mg, 0.474 mmol, 1 equiv), trimethyl-1,3,5,2,4,6-trioxatriborinane (119.1 mg, 0.948 mmol, 2 equiv), Pd(dppf)Cl₂ (34.7 mg, 0.047 mmol, 0.1 equiv) and K₂CO₃ (196.7 mg, 1.422 mmol, 3 equiv) in DMF (2.5 mL) was stirred for 3 h at 100° C. under nitrogen atmosphere. The resulting mixture was diluted with water (50 mL). The resulting mixture was extracted with EtOAc (3×30 mL). The combined organic layers were washed with brine (3×50 mL), dried over anhydrous Na₂SO₄. After filtration, the filtrate was concentrated under reduced pressure. The residue was purified by reversed-phase flash chromatography to afford tert-butyl 4-fluoro-5-methylisoindoline-2-carboxylate (100 mg, 84%) as a grey solid. LC-MS (ES, m/z)=237.1 [M-t-Bu+ACN]⁺.

Step 8. Preparation of 4-fluoro-5-methylisoindoline. A mixture of tert-butyl 4-fluoro-5-methylisoindoline-2-carboxylate (100.0 mg, 0.398 mmol, 1 equiv) in dioxane (5 mL) was added HCl (gas) in 1,4-dioxane (2.5 mL) dropwise at 0° C. The resulting mixture was stirred for 2 h at room temperature. The resulting mixture was concentrated under reduced pressure. This resulted in 4-fluoro-5-methylisoindoline (90 mg, crude) as a brown solid. The crude product was used in the next step directly without further purification. LC-MS (ES, m/z)=152.1 [M+1]⁺.

Step 9. Preparation of 3-(5-(4-fluoro-5-methylisoindoline-2-carbonyl)-1-oxoisoindolin-2-yl)piperidine-2,6-dione. A mixture of 4-fluoro-5-methylisoindoline (90.0 mg, 0.595 mmol, 1 equiv) and 2-(2,6-dioxopiperidin-3-yl)-1-oxo-3H-isoindole-5-carboxylic acid (223.1 mg, 0.773 mmol, 1.3 equiv) in DMF (1.4 mL) was added HATU (339.5 mg, 0.892 mmol, 1.5 equiv) and DIEA (230.8 mg, 1.785 mmol, 3 equiv) at room temperature. The resulting mixture was stirred for 2 h at room temperature under nitrogen atmosphere. The resulting mixture was diluted with water (30 mL). The resulting mixture was extracted with EtOAc (3×30 mL). The combined organic layers were washed with brine (2×50 mL), dried over anhydrous Na₂SO₄. After filtration, the filtrate was concentrated under reduced pressure. The crude product was purified by Prep-HPLC to afford 3-(5-(4-fluoro-5-methylisoindoline-2-carbonyl)-1-oxoisoindolin-2-yl)piperidine-2,6-dione (6.4 mg, 2.5%) as an off-white solid. LC-MS (ES, m/z)=422.1 [M+1]⁺. ¹H NMR (400 MHz, DMSO-d₆) δ 11.01 (s, 1H), 7.97-7.82 (m, 2H), 7.73 (t, J=6.5 Hz, 1H), 7.23 (dt, J=15.1, 7.4 Hz, 1H), 6.99 (d, J=7.7 Hz, 1H), 5.16 (dd, J=13.3, 5.1 Hz, 1H), 4.90 (d, J=8.7 Hz, 2H), 4.78 (d, J=18.2 Hz, 2H), 4.54 (dd, J=17.5, 6.8 Hz, 1H), 4.41 (dd, J=17.6, 5.3 Hz, 1H), 2.93 (ddd, J=18.0, 13.6, 5.3 Hz, 1H), 2.61 (d, J=17.6 Hz, 1H), 2.42 (dt, J=14.1, 7.1 Hz, 1H), 2.23 (d, J=12.4 Hz, 3H), 2.15-2.00 (m, 1H), 1.28 (d, J=39.9 Hz, 1H).

### Example 150

### Synthesis of 2-(2-(2,6-dioxopiperidin-3-yl)-1-oxoisoindoline-5-carbonyl)-4-fluoroisoindoline-5-carbonitrile

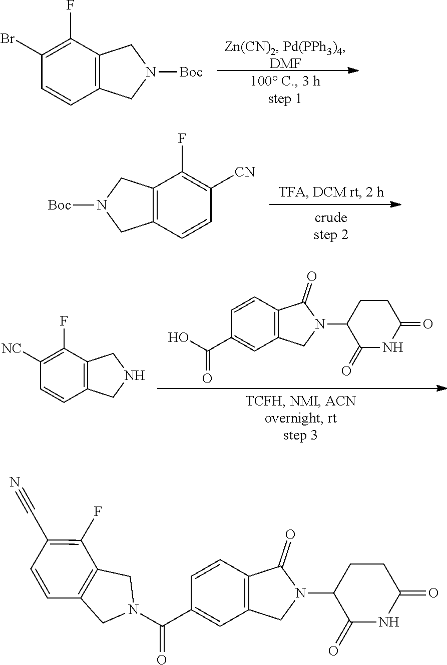 [SMILES: CC(C)(C)OC(=O)N1Cc2ccc(Br)c(F)c2C1 ; CC(C)(C)OC(=O)N1Cc2ccc(C#N)c(F)c2C1 ; N#Cc1ccc2c(c1F)CNC2 ; O=C1CCC(N2Cc3cc(C(=O)O)ccc3C2=O)C(=O)N1 ; N#Cc1ccc2c(c1F)CN(C(=O)c1ccc3c(c1)CN(C1CCC(=O)NC1=O)C3=O)C2]

Step 1. Preparation of tert-butyl 5-cyano-4-fluoroisoindoline-2-carboxylate. A mixture of tert-butyl 6-bromo-4-fluoro-1,3-dihydroisoindole-2-carboxylate (600.0 mg, 1.898 mmol, 1 equiv), Zn(CN)₂ (779.9 mg, 6.643 mmol, 3.5 equiv) and Pd(dppf)Cl₂ (138.9 mg, 0.190 mmol, 0.1 equiv) in DMF (9 mL) was stirred for overnight at 100° C. under nitrogen atmosphere. The resulting mixture was diluted with water (100 mL). The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 5-cyano-4-fluoroisoindoline-2-carboxylate (430 mg, 72%) as a brown solid. LC-MS (ES, m/z)=248.1 [M-t-Bu+ACN]⁺.

Step 2. Preparation of 4-fluoroisoindoline-5-carbonitrile. A mixture of tert-butyl 5-cyano-4-fluoroisoindoline-2-carboxylate (430.0 mg, 1.639 mmol, 1 equiv) in DCM (5 mL) was added TFA (1 mL) dropwise at 0° C. The resulting mixture was stirred for 2 h at room temperature. The resulting mixture was concentrated under vacuum to afford 4-fluoroisoindoline-5-carbonitrile (400.0 mg, crude) as a brown solid. The residue was used in the next step directly without further purification. LC-MS (ES, m/z)=163.1 [M+1]⁺.

Step 3. Preparation of 2-(2-(2,6-dioxopiperidin-3-yl)-1-oxoisoindoline-5-carbonyl)-4-fluoroisoindoline-5-carbonitrile. A mixture of 4-fluoroisoindoline-5-carbonitrile (400.0 mg, 2.467 mmol, 1 equiv) and 2-(2,6-dioxopiperidin-3-yl)-1-oxo-3H-isoindole-5-carboxylic acid (924.3 mg, 3.207 mmol, 1.3 equiv) in acetonitrile (6 ml) was added NMI (810.1 mg, 9.868 mmol, 4 equiv) and TCFH (1.38 g, 4.934 mmol, 2 equiv) in portions at room temperature. The resulting mixture was stirred overnight at room temperature under air atmosphere. The resulting mixture was diluted with water (100 mL). The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford 2-(2-(2,6-dioxopiperidin-3-yl)-1-oxoisoindoline-5-carbonyl)-4-fluoroisoindoline-5-carbonitrile (20 mg, 1.81%) as an off-white solid. LC-MS (ES, m/z)=433.1 [M+1]⁺. ¹H NMR (400 MHz, DMSO-d₆) δ 11.01 (s, 1H), 7.87 (td, J=12.6, 10.8, 7.5 Hz, 3H), 7.73 (d, J=7.9 Hz, 1H), 7.42 (dd, J=66.7, 7.9 Hz, 1H), 5.16 (dd, J=13.3, 5.0 Hz, 1H), 4.96 (dd, J=42.3, 6.5 Hz, 4H), 4.64-4.34 (m, 2H), 2.93 (ddd, J=18.3, 13.7, 5.4 Hz, 1H), 2.70-2.58 (m, 1H), 2.42 (td, J=13.3, 4.5 Hz, 1H), 2.15-1.97 (m, 2H).

### Example 151

### Synthesis of 3-(1-oxo-5-(4-(trifluoromethoxy)isoindoline-2-carbonyl)isoindolin-2-yl)piperidine-2,6-dione

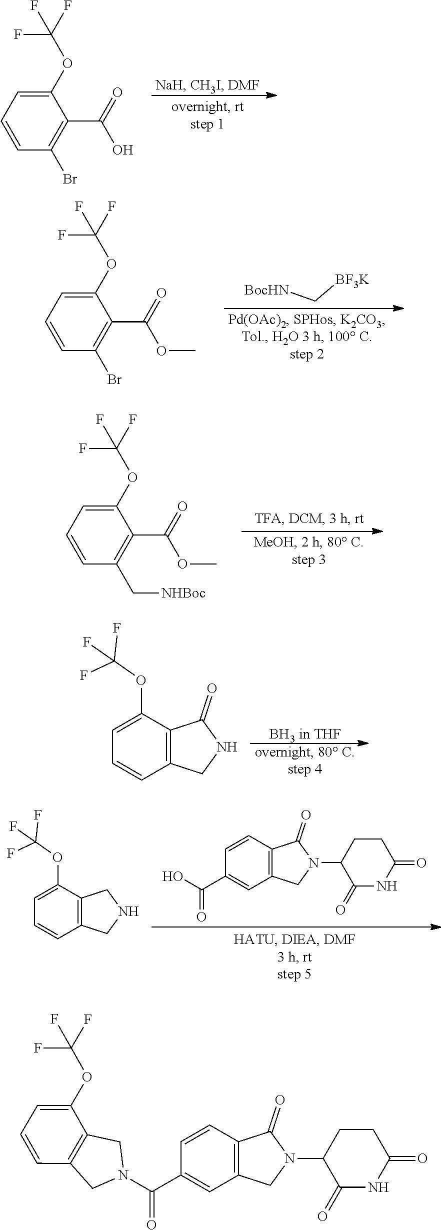

Step 1. Preparation of methyl 2-bromo-6-(trifluoromethoxy)benzoate. A solution of 2-bromo-6-(trifluoromethoxy)benzoic acid (6 g, 21.051 mmol, 1 equiv) in DMF (60 mL) was treated with NaH (1.68 g, 42.102 mmol, 2 equiv, 60%) for 30 min at 0° C. under nitrogen atmosphere followed by the addition of CH₃I (8.96 g, 63.153 mmol, 3 equiv) at 0° C. The resulting mixture was stirred overnight at room temperature. The reaction was quenched by the addition of Water/Ice (5 mL) at 0° C. The resulting mixture was diluted with water (500 mL) and extracted with EtOAc (3×200 mL). The combined organic layers were washed with brine (2×500 mL), dried over anhydrous Na₂SO₄. After filtration, the filtrate was concentrated under reduced pressure. The residue was purified by reversed-phase flash chromatography to afford methyl 2-bromo-6-(trifluoromethoxy)benzoate (2 g, 32%) as a brown oil. LC-MS (ES, m/z)=no mass detected.

Step 2. Preparation of methyl 2-(((tert-butoxycarbonyl)amino)methyl)-6-(trifluoromethoxy)benzoate. To a stirred mixture of methyl 2-bromo-6-(trifluoromethoxy)benzoate (1.26 g, 4.213 mmol, 1 equiv) and tert-butyl N-[(trifluoro-γ⁴-boranyl)methyl]carbamate potassium (1.5 g, 6.319 mmol, 1.5 equiv) in Toluene (12.60 mL) and H₂O (3.15 mL) were added Pd(OAc)₂ (94.6 mg, 0.421 mmol, 0.1 equiv), K₂CO₃ (1.75 g, 12.639 mmol, 3 equiv), and SPhos (345.9 mg, 0.843 mmol, 0.2 equiv) at room temperature under nitrogen atmosphere. The resulting mixture was stirred for 3 h at 100° C. under nitrogen atmosphere. The resulting mixture was diluted with water (200 mL) and extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford methyl 2-(((tert-butoxycarbonyl)amino)methyl)-6-(trifluoromethoxy)benzoate (1 g, 68%) as a brown oil. LC-MS (ES, m/z)=294.0 [M-tBu+1]⁺.

Step 3. Preparation of 7-(trifluoromethoxy)isoindolin-1-one. To a stirred mixture of methyl 2-(((tert-butoxycarbonyl)amino)methyl)-6-(trifluoromethoxy)benzoate (1 g, 2.863 mmol, 1 equiv) in DCM (20 mL) was added TFA (4 mL, 53.853 mmol, 18.81 equiv) dropwise at 0° C. The resulting mixture was stirred for 3 h at room temperature. The resulting mixture was concentrated under reduced pressure. To the above mixture was added CH₃OH (20 mL) at room temperature. The resulting mixture was stirred for an additional 2 h at 80° C. The resulting mixture was concentrated under reduced pressure. The residue was purified by reversed-phase flash chromatography to afford 7-(trifluoromethoxy)isoindolin-1-one (600 mg, 97%) as a white oil. LC-MS (ES, m/z)=218.0 [M+1]⁺.

Step 4. Preparation of 4-(trifluoromethoxy)isoindoline. To a stirred mixture of 7-(trifluoromethoxy)isoindolin-1-one (500.0 mg, 2.303 mmol, 1 equiv) in THF (10.00 mL) was added BH₃-THF (9.99 mL, 9.995 mmol, 4.34 equiv) at 0° C. under nitrogen atmosphere. The resulting mixture was stirred overnight at 80° C. under nitrogen atmosphere. The reaction was quenched by the addition of MeOH (2 mL) at 0° C. The resulting mixture was concentrated under reduced pressure. The residue was purified by reversed-phase flash chromatography to afford 4-(trifluoromethoxy)isoindoline (400 mg, 86%) as a white solid. LC-MS (ES, m/z)=204.2 [M+1]⁺.

Step 5. Preparation of 3-(1-oxo-5-(4-(trifluoromethoxy)isoindoline-2-carbonyl)isoindolin-2-yl)piperidine-2,6-dione. To a stirred mixture of 2-(2,6-dioxopiperidin-3-yl)-1-oxo-3H-isoindole-5-carboxylic acid (100.0 mg, 0.347 mmol, 1 equiv) and 4-(trifluoromethoxy)isoindoline (105.7 mg, 0.520 mmol, 1.5 equiv) in DMF (1 mL) were added HATU (197.9 mg, 0.520 mmol, 1.5 equiv) and DIEA (134.5 mg, 1.041 mmol, 3 equiv) at room temperature. The resulting mixture was stirred for 3 h at room temperature. The resulting mixture was diluted with water (30 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(1-oxo-5-(4-(trifluoromethoxy)isoindoline-2-carbonyl)isoindolin-2-yl)piperidine-2,6-dione (50 mg, 30%) as a white solid. LC-MS (ES, m/z)=474.0 [M+1]⁺. ¹H NMR (400 MHz, DMSO-d₆) δ 11.01 (s, 1H), 7.88-7.80 (m, 2H), 7.78-7.70 (m, 1H), 7.39 (dq, J=59.4, 8.4, 7.9 Hz, 3H), 5.20-5.11 (m, 1H), 4.96 (d, J=11.2 Hz, 2H), 4.84 (d, J=8.8 Hz, 2H), 4.54 (dd, J=17.6, 5.2 Hz, 1H), 4.41 (dd, J=17.6, 4.4 Hz, 1H), 3.00-2.86 (m, 1H), 2.66-2.57 (m, 1H), 2.43 (d, J=13.1 Hz, 1H), 2.05 (d, J=8.9 Hz, 1H).

### Example 152

### Synthesis of 3-(5-(4-chloro-6-fluoroisoindoline-2-carbonyl)-1-oxoisoindolin-2-yl)piperidine-2,6-dione

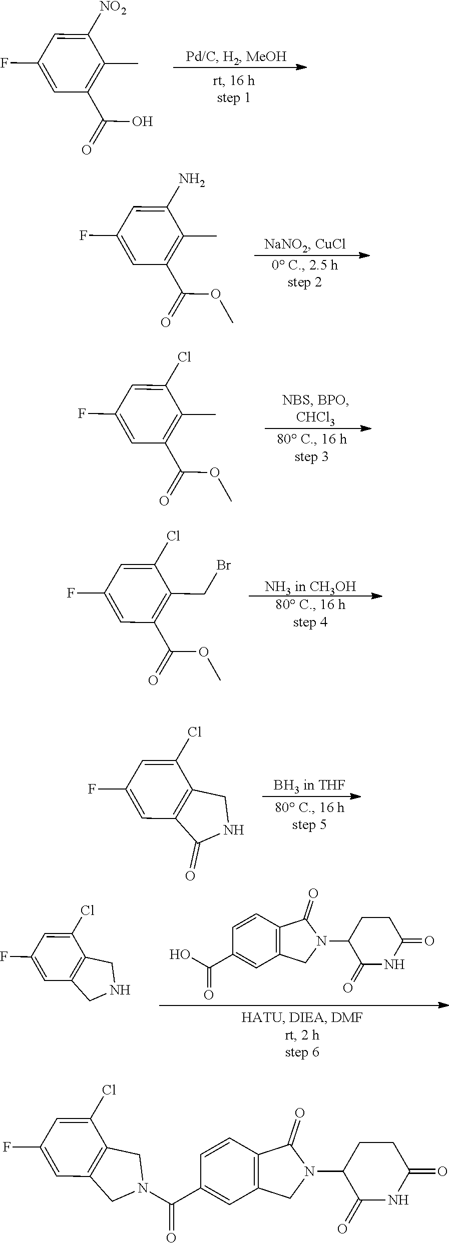

Step 1. Preparation of methyl 3-amino-5-fluoro-2-methylbenzoate. To a solution of methyl 5-fluoro-2-methyl-3-nitrobenzoate (2 g, 9.382 mmol, 1 equiv) in MeOH (30 mL) was added Pd/C (Palladium 10% on Carbon (wetted with ca. 55% Water), 500 mg) in a 100 mL round-bottom flask. The mixture was hydrogenated at room temperature overnight under hydrogen atmosphere using a hydrogen balloon, filtered through a Celite pad and concentrated under reduced pressure to afford methyl 3-amino-5-fluoro-2-methylbenzoate (1.6 g, 93%) as a yellow oil. LC-MS (ES, m/z)=184.05 [M+1]⁺.

Step 2. Preparation of methyl 3-chloro-5-fluoro-2-methylbenzoate. To a stirred solution of methyl 3-amino-5-fluoro-2-methylbenzoate (1.5 g, 8.189 mmol, 1 equiv) in HCl (6M) (150 mL) was added NaNO₂ (0.7 g, 10.146 mmol, 1.24 equiv) in portions at 0° C. under nitrogen atmosphere. The resulting mixture was stirred for 10 min at 0° C. under nitrogen atmosphere. To the above mixture was added CuCl (1.2 g, 12.121 mmol, 1.48 equiv) in portions over 10 min at 0° C. The resulting mixture was stirred for an additional 2 h at 0° C. The resulting mixture was extracted with DCM (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (2:1) to afford methyl 3-chloro-5-fluoro-2-methylbenzoate (800 mg, 48%) as a Brown yellow solid. LC-MS (ES, m/z)=203.0 [M+1]⁺.

Step 3. Preparation of methyl 2-(bromomethyl)-3-chloro-5-fluorobenzoate. To a stirred mixture of methyl 3-chloro-5-fluoro-2-methylbenzoate (700 mg, 3.455 mmol, 1 equiv) and AIBN (56.7 mg, 0.345 mmol, 0.10 equiv) in chloroform (10 mL) were added NBS (922.4 mg, 5.182 mmol, 1.50 equiv) in portions at room temperature. The resulting mixture was stirred overnight at 80° C. under nitrogen atmosphere. The mixture was allowed to cool down to room temperature. The resulting mixture was concentrated under vacuum. The residue was purified by silica gel column chromatography, eluted with PE/EA (3:1) to afford methyl 2-(bromomethyl)-3-chloro-5-fluorobenzoate (800 mg, 82%) as a brown oil. LC-MS (ES, m/z)=281.0 [M+1]⁺.

Step 4. Preparation of 4-chloro-6-fluoroisoindolin-1-one. A mixture of methyl 2 (bromomethyl)-3-chloro-5-fluorobenzoate (770 mg, 2.735 mmol, 1 equiv) in NH₃ (g) in MeOH (15 mL, 7. OM) was stirred for 2 h at room temperature under nitrogen atmosphere. The resulting mixture was stirred for additional overnight at 80° C. The mixture was allowed to cool down to room temperature. The resulting mixture was concentrated under vacuum. The residue was purified by silica gel column chromatography, eluted with DCM/MeOH (10:1) to afford 4-chloro-6-fluoroisoindolin-1-one (400 mg, 79%) as a yellow solid. LC-MS (ES, m/z)=186.2 [M+1]⁺.

Step 5. Preparation of 4-chloro-6-fluoroisoindoline. To a stirred solution of 4-chloro-6-fluoroisoindolin-1-one (300 mg, 1.617 mmol, 1 equiv) in THF (3 mL) was added BH₃-THF (8.1 mL, 8.100 mmol, 5.01 equiv) dropwise at 0° C. under nitrogen atmosphere. The resulting mixture was stirred overnight at 80° C. under nitrogen atmosphere. The mixture was allowed to cool down to room temperature. The reaction was quenched with MeOH at 0° C. The resulting mixture was concentrated under vacuum. The residue was purified by reversed-phase flash chromatography to afford 4-chloro-6-fluoroisoindoline (200 mg, 72%) as a yellow solid. LC-MS (ES, m/z)=172.15 [M+1]⁺.

Step 6. Preparation of 3-(5-(4-chloro-6-fluoroisoindoline-2-carbonyl)-1-oxoisoindolin-2-yl)piperidine-2,6-dione. To a stirred mixture of 4-chloro-6-fluoroisoindoline (89.3 mg, 0.520 mmol, 1.50 equiv) and 2-(2,6-dioxopiperidin-3-yl)-1-oxo-3H-isoindole-5-carboxylic acid (100 mg, 0.347 mmol, 1.00 equiv) in dimethylformamide (2 mL) was added DIEA (134.5 mg, 1.041 mmol, 3.00 equiv) at room temperature under air atmosphere. To the above mixture was added HATU (197.9 mg, 0.520 mmol, 1.50 equiv) at room temperature. The resulting mixture was stirred for an additional 2 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to afford (60 mg) crude product. The crude product was purified by Prep-HPLC to afford 3-(5-(4-chloro-6-fluoroisoindoline-2-carbonyl)-1-oxoisoindolin-2-yl)piperidine-2,6-dione (27 mg, 18%) as an off-white solid. LC-MS (ES, m/z)=442.05 [M+1]⁺. ¹H NMR (400 MHz, DMSO-d₆) δ 11.02 (s, 1H), 7.87-7.80 (m, 2H), 7.73 (dd, J=8.4, 7.0 Hz, 1H), 7.44-7.14 (m, 2H), 5.16 (dd, J=13.3, 5.1 Hz, 1H), 4.97 (s, 1H), 4.85 (s, 2H), 4.73 (s, 1H), 4.54 (dd, J=17.6, 6.6 Hz, 1H), 4.41 (dd, J=17.6, 4.8 Hz, 1H), 2.93 (ddd, J=17.2, 13.5, 5.3 Hz, 1H), 2.66-2.57 (m, 1H), 2.41 (t, J=13.0 Hz, 1H), 2.05 (d, J=6.2 Hz, 1H).

### Example 153

### Synthesis of 3-(6-(4-(trifluoromethoxy)isoindoline-2-carbonyl)benzo[d]oxazol-2-yl)piperidine-2,6-dione

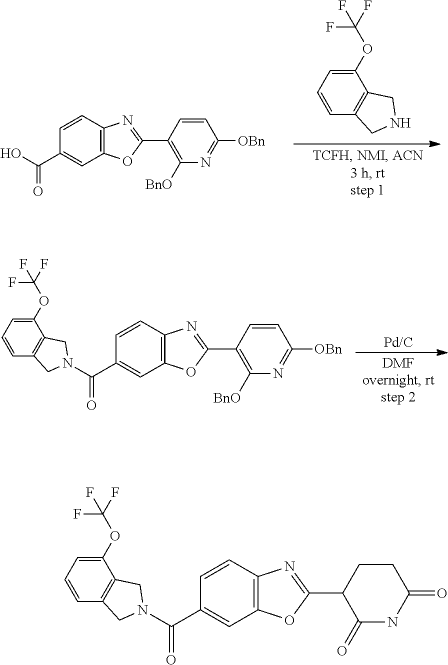 [SMILES: O=C(O)c1ccc2nc(-c3ccc(OCc4ccccc4)nc3OCc3ccccc3)oc2c1 ; FC(F)(F)Oc1cccc2c1CNC2 ; O=C(c1ccc2nc(-c3ccc(OCc4ccccc4)nc3OCc3ccccc3)oc2c1)N1Cc2cccc(OC(F)(F)F)c2C1 ; O=C1CCC(c2nc3ccc(C(=O)N4Cc5cccc(OC(F)(F)F)c5C4)cc3o2)C(=O)N1]

Step 1. Preparation of (2-(2,6-bis(benzyloxy)pyridin-3-yl)benzo[d]oxazol-6-yl)(4-(trifluoromethoxy)isoindolin-2-yl)methanone. To a stirred mixture of 2-[2,6-bis(benzyloxy)pyridin-3-yl]-1,3-benzoxazole-6-carboxylic acid (80 mg, 0.177 mmol, 1 equiv) and 4-(trifluoromethoxy)isoindoline (107.7 mg, 0.531 mmol, 3 equiv) in ACN (2 mL) were added TCFH (74.4 mg, 0.265 mmol, 1.5 equiv) and NMI (58.0 mg, 0.708 mmol, 4 equiv) at room temperature. The resulting mixture was stirred for 3 h at room temperature. The resulting mixture was diluted with water (20 mL) and extracted with EtOAc (3×10 mL). The combined organic layers were washed with brine (2×20 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(4-(trifluoromethoxy)isoindolin-2-yl)methanone (100 mg, 89%) as a white solid. LC-MS (ES, m/z)=638.0 [M+1]⁺.

Step 2. Preparation of 3-(6-(4-(trifluoromethoxy)isoindoline-2-carbonyl)benzo[d]oxazol-2-yl)piperidine-2,6-dione. To a stirred mixture of (2-(2,6-bis(benzyloxy)pyridin-3-yl)benzo[d]oxazol-6-yl)(4-(trifluoromethoxy)isoindolin-2-yl)methanone (90 mg, 0.141 mmol, 1 equiv) in dimethylformamide (1 mL) was added Pd/C (45 mg, Palladium 10% on Carbon (wetted with ca. 55% Water)) at room temperature. The resulting mixture was stirred overnight at room temperature under hydrogen atmosphere. The resulting mixture was filtered, the filter cake was washed with EtOAc (3×5 mL). The filtrate was concentrated under reduced pressure. The crude product was purified by Prep-HPLC to afford 3-(6-(4-(trifluoromethoxy)isoindoline-2-carbonyl)benzo[d]oxazol-2-yl)piperidine-2,6-dione (4.8 mg, 7.40%) as an off-white solid. LC-MS (ES, m/z)=460.05 [M+1]⁺. ¹H NMR (400 MHz, DMSO-d₆) δ 11.17 (s, 1H), 8.08 (s, 1H), 7.86 (d, J=8.1 Hz, 1H), 7.65 (d, J=8.2 Hz, 1H), 7.49-7.29 (m, 3H), 4.97 (d, J=11.4 Hz, 2H), 4.89 (d, J=9.0 Hz, 2H), 4.57 (dd, J=11.4, 5.0 Hz, 1H), 2.77-2.65 (m, 2H), 2.45 (s, 1H), 2.36 (d, J=5.0 Hz, 1H).

### Example 154

### Synthesis of 3-(5-(6′-fluorospiro[azetidine-3,3′-indoline]-1′-carbonyl)-1-oxoisoindolin-2-yl)piperidine-2,6-dione

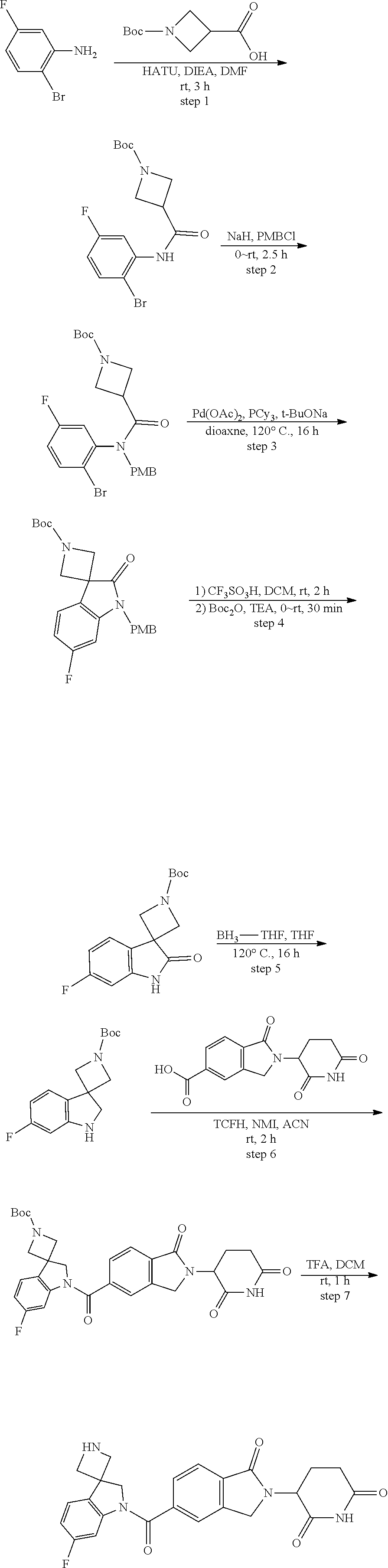

Step 1. Preparation of tert-butyl 3-((2-bromo-5-fluorophenyl)carbamoyl)azetidine-1-carboxylate. To a stirred mixture of 2-bromo-5-fluoroaniline (5 g, 26.314 mmol, 1 equiv) and 1-(tert-butoxycarbonyl)azetidine-3-carboxylic acid (5.29 g, 26.314 mmol, 1 equiv) and DIEA (10.2 g, 78.942 mmol, 3 equiv) in DMF (75 mL) was added HATU (15 g, 39.471 mmol, 1.5 equiv) in portions at room temperature. The resulting mixture was stirred for 3 h at room temperature. The resulting mixture was diluted with water (500 mL) and extracted with EtOAc (3×200 mL). The combined organic layers were washed with brine (2×500 mL), dried over anhydrous Na₂SO₄. After filtration, the filtrate was concentrated under reduced pressure. The residue was purified by reversed-phase flash chromatography to afford tert-butyl 3-((2-bromo-5-fluorophenyl)carbamoyl)azetidine-1-carboxylate (6 g, 61%) as a yellow oil. LC-MS (ES, m/z)=373.1 [M+1]⁺.

Step 2. Preparation of tert-butyl 3-((2-bromo-5-fluorophenyl)(4-methoxybenzyl)carbamoyl)azetidine-1-carboxylate. A solution of tert-butyl 3-((2-bromo-5-fluorophenyl)carbamoyl)azetidine-1-carboxylate (4 g, 10.717 mmol, 1 equiv) in DMF (60 mL) was treated with NaH (1.71 g, 42.868 mmol, 4 equiv, 60%) for 30 min at 0° C. under nitrogen atmosphere followed by the addition of PMBCl (3.36 g, 21.434 mmol, 2 equiv) at 0° C. The resulting mixture was stirred for 2 h at room temperature under nitrogen atmosphere. The reaction was quenched by water at 0° C. and extracted with EtOAc (3×200 mL). The combined organic layers were washed with brine (2×200 mL), dried over anhydrous Na₂SO₄. After filtration, the filtrate was concentrated under reduced pressure. The residue was purified by reversed-phase flash chromatography to afford tert-butyl 3-((2-bromo-5-fluorophenyl)(4-methoxybenzyl)carbamoyl)azetidine-1-carboxylate (2 g, 38%) as an off-white solid. LC-MS (ES, m/z)=493.0 [M+1]⁺.

Step 3. Preparation of tert-butyl 6′-fluoro-1′-(4-methoxybenzyl)-2′-oxospiro[azetidine-3,3′-indoline]-1-carboxylate. To a stirred mixture of tert-butyl 3-((2-bromo-5-fluorophenyl)(4-methoxybenzyl)carbamoyl)azetidine-1-carboxylate (1 g, 2.027 mmol, 1 equiv) and PCy₃ (56.8 mg, 0.203 mmol, 0.1 equiv) in dioxane (15 ml) were added Pd(OAc)₂ (22.8 mg, 0.1 mmol, 0.05 equiv) and t-BuONa (292.2 mg, 3.041 mmol, 1.5 equiv) at room temperature under nitrogen atmosphere. The resulting mixture was stirred overnight at 120° C. under nitrogen atmosphere. The mixture was allowed to cool down to room temperature. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6′-fluoro-1′-(4-methoxybenzyl)-2′-oxospiro[azetidine-3,3′-indoline]-1-carboxylate (600 mg, 72%) as a colorless oil. LC-MS (ES, m/z)=413.0 [M+1]⁺.

Step 4. Preparation of tert-butyl 6′-fluoro-2′-oxospiro[azetidine-3,3′-indoline]-1-carboxylate. To a stirred solution of tert-butyl 6′-fluoro-1′-(4-methoxybenzyl)-2′-oxospiro[azetidine-3,3′-indoline]-1-carboxylate (500 mg, 1.212 mmol, 1 equiv) in DCM (10 mL) were added CF₃SO₃H (0.43 mL, 4.848 mmol, 4 equiv) dropwise at room temperature. The resulting mixture was stirred for 2 h at room temperature under nitrogen atmosphere. To the above mixture was added TEA (1.35 mL, 9.696 mmol, 8 equiv) and Boc₂O (0.31 mL, 1.449 mmol, 1.20 equiv) dropwise over 5 min at 0° C. The resulting mixture was stirred for an additional 30 min at room temperature. The mixture is diluted with saturated aqueous sodium bicarbonate, followed by extraction with DCM (3×50 mL). The organic layer is dried over Na₂SO₄, filtered and concentrated. The residue is purified by column chromatography on silica-gel eluting with 0-50% EA in PE to afford tert-butyl 6′-fluoro-2′-oxospiro[azetidine-3,3′-indoline]-1-carboxylate (200 mg, 56%) as a yellow oil. LC-MS (ES, m/z)=291.2 [M+1]⁺.

Step 5. Preparation of tert-butyl 6′-fluorospiro[azetidine-3,3′-indoline]-1-carboxylate. To a stirred solution of tert-butyl 6′-fluoro-2′-oxospiro[azetidine-3,3′-indoline]-1-carboxylate (180 mg, 0.616 mmol, 1 equiv) in THF (2 mL) was added BH₃-THF (3.08 mL, 3.080 mmol, 5 equiv) dropwise at 0° C. under nitrogen atmosphere. The resulting mixture was stirred overnight at 60° C. under nitrogen atmosphere. The mixture was allowed to cool down to room temperature and quenched with MeOH (1 mL) at room temperature and concentrated under vacuum. The residue was purified by reversed-phase flash chromatography to afford tert-butyl 6′-fluorospiro[azetidine-3,3′-indoline]-1-carboxylate (60 mg, 35%) as an off-white solid. LC-MS (ES, m/z)=320.3 [M+1]⁺.

Step 6. Preparation of tert-butyl 1′-(2-(2,6-dioxopiperidin-3-yl)-1-oxoisoindoline-5-carbonyl)-6′-fluorospiro[azetidine-3,3′-indoline]-1-carboxylate. To a stirred mixture of tert-butyl 6′-fluorospiro[azetidine-3,3′-indoline]-1-carboxylate (50 mg, 0.180 mmol, 1 equiv) and 2-(2,6-dioxopiperidin-3-yl)-1-oxo-3H-isoindole-5-carboxylic acid (51.8 mg, 0.180 mmol, 1.00 equiv) in ACN (1.5 mL) was added NMI (73.8 mg, 0.899 mmol, 5.00 equiv) at room temperature. To the above mixture was added TCFH (75.6 mg, 0.269 mmol, 1.50 equiv) in portions at room temperature. The resulting mixture was stirred for an additional 2 h at room temperature. The resulting mixture was diluted with water (20 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by reversed-phase flash chromatography to afford tert-butyl 1′-(2-(2,6-dioxopiperidin-3-yl)-1-oxoisoindoline-5-carbonyl)-6′-fluorospiro[azetidine-3,3′-indoline]-1-carboxylate (60 mg, 61%) as an off-white solid. LC-MS (ES, m/z)=493.2 [M+1]⁺.

Step 7. Preparation of 3-(5-(6′-fluorospiro[azetidine-3,3′-indoline]-1′-carbonyl)-1-oxoisoindolin-2-yl)piperidine-2,6-dione. To a stirred solution of tert-butyl 1′-(2-(2,6-dioxopiperidin-3-yl)-1-oxoisoindoline-5-carbonyl)-6′-fluorospiro[azetidine-3,3′-indoline]-1-carboxylate (50 mg, 0.091 mmol, 1 equiv) in DCM (1 mL) was added TFA (0.2 mL) dropwise at room temperature under nitrogen atmosphere. The resulting mixture was stirred for 1 h at room temperature under nitrogen atmosphere. The resulting mixture was concentrated under vacuum. The residue was purified by reversed-phase flash chromatography to afford 3-(5-(6′-fluorospiro[azetidine-3,3′-indoline]-1′-carbonyl)-1-oxoisoindolin-2-yl)piperidine-2,6-dione (38 mg, 92%) as an off-white solid. LC-MS (ES, m/z)=449.15 [M+1]⁺. ¹H NMR (400 MHz, DMSO-d₆) δ 11.03 (s, 1H), 8.86 (s, 1H), 8.62 (s, 1H), 7.98-7.79 (m, 3H), 7.75 (d, J=7.8 Hz, 1H), 7.13 (td, J=8.8, 2.6 Hz, 1H), 5.17 (dd, J=13.3, 5.1 Hz, 1H), 4.56 (d, J=17.6 Hz, 1H), 4.47-4.31 (m, 3H), 4.18 (t, J=5.7 Hz, 3H), 2.94 (ddd, J=17.2, 13.5, 5.4 Hz, 1H), 2.67-2.58 (m, 1H), 2.43 (td, J=13.2, 4.6 Hz, 1H), 2.05 (dtd, J=10.9, 5.6, 3.0 Hz, 1H).

### Example 155

### Synthesis of 3-(5-(4-fluoro-6-methylisoindoline-2-carbonyl)-1-oxoisoindolin-2-yl)piperidine-2,6-dione

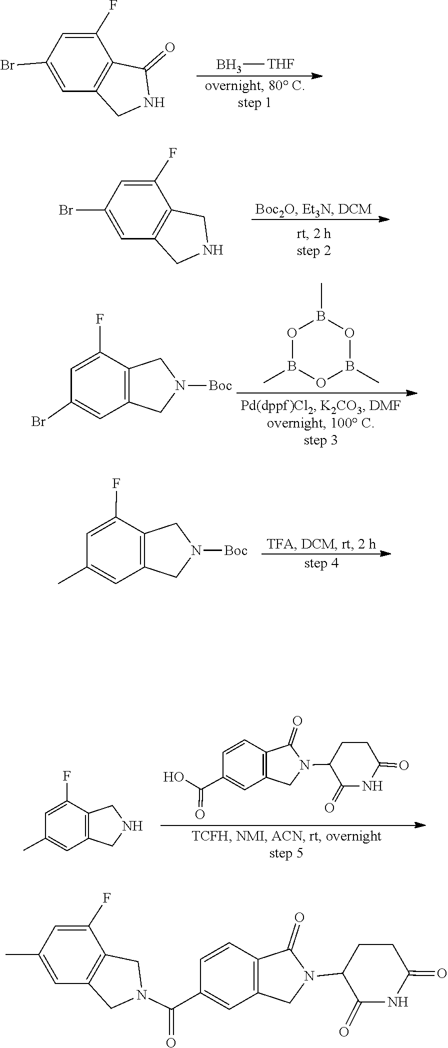

Step 1. Preparation of 6-bromo-4-fluoroisoindoline. To a stirred mixture of 5-bromo-7-fluoro-2,3-dihydroisoindol-1-one (1.5 g, 6.521 mmol, 1 equiv) in THF (12 ml) was added BH₃-THF (33 mL, 344.822 mmol) dropwise at 0° C. under nitrogen atmosphere. The resulting mixture was stirred overnight at 80° C. under nitrogen atmosphere. The reaction was quenched with water at 0° C. The resulting mixture was acidified with 1M HCl (aq.) and concentrated under reduced pressure. The residue was purified by reversed-phase flash chromatography to afford 6-bromo-4-fluoroisoindoline hydrochloride (2 g, crude) as a blue solid. LC-MS (ES, m/z)=216.0 [M+1]⁺.

Step 2. Preparation of tert-butyl 6-bromo-4-fluoroisoindoline-2-carboxylate. A mixture of 6-bromo-4-fluoroisoindoline hydrochloride (2 g, 9.257 mmol, 1 equiv) and Boc₂O (4.04 g, 18.514 mmol, 2 equiv) and Et₃N (2.81 g, 27.771 mmol, 3 equiv) in DCM (30 mL) was stirred for 2 h at room temperature under air atmosphere. The resulting mixture was diluted with water and extracted with DCM (3×200 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 6-bromo-4-fluoroisoindoline-2-carboxylate (750 mg, 26%) as a brown oil. LC-MS (ES, m/z)=301.0 [M-t-Bu+ACN]⁺.

Step 3. Preparation of tert-butyl 4-fluoro-6-methylisoindoline-2-carboxylate. A mixture of tert-butyl 6-bromo-4-fluoroisoindoline-2-carboxylate (400 mg, 1.265 mmol, 1 equiv.), trimethyl-1,3,5,2,4,6-trioxatriborinane (317.6 mg, 2.530 mmol, 2 equiv), Pd(dppf)Cl₂ (92.6 mg, 0.127 mmol, 0.1 equiv) and K₂CO₃ (524.6 mg, 3.795 mmol, 3 equiv) in DMF (6 ml) was stirred for overnight at 100° C. under nitrogen atmosphere. The resulting mixture was diluted with water and extracted with EA (3×50 mL). The combined organic layers were washed with brine (3×100 mL), dried over anhydrous Na₂SO₄. After filtration, the filtrate was concentrated under reduced pressure. The filtrate was concentrated under reduced pressure. The residue was purified by reversed-phase flash chromatography to afford tert-butyl 4-fluoro-6-methylisoindoline-2-carboxylate (230 mg, 72%) as a grey solid. LC-MS (ES, m/z)=237.1 [M-t-Bu+ACN]⁺.

Step 4. Preparation of 4-fluoro-6-methylisoindoline. To a stirred mixture of tert-butyl 4-fluoro-6-methylisoindoline-2-carboxylate (230 mg, 0.915 mmol, 1 equiv) in DCM (2.5 mL) was added TFA (0.5 mL) dropwise at 0° C. under air atmosphere. The resulting mixture was stirred for 2 h at room temperature under the air atmosphere. The resulting mixture was concentrated under vacuum to afford 4-fluoro-6-methylisoindoline (190 mg, crude) as a black solid. The crude product was used in the next step directly without further purification. LC-MS (ES, m/z)=152.05 [M+1]⁺.

Step 5. Preparation of 3-(5-(4-fluoro-6-methylisoindoline-2-carbonyl)-1-oxoisoindolin-2-yl)piperidine-2,6-dione. A mixture of 4-fluoro-6-methylisoindoline (180 mg, 1.191 mmol, 1 equiv), 2-(2,6-dioxopiperidin-3-yl)-1-oxo-3H-isoindole-5-carboxylic acid (446.2 mg, 1.548 mmol, 1.3 equiv), NMI (391.0 mg, 4.764 mmol, 4 equiv) and TCFH (668.1 mg, 2.382 mmol, 2 equiv) in acetonitrile (2.7 ml) was stirred overnight at room temperature under air atmosphere. The resulting mixture was diluted with water and extracted with EtOAc (3×30 mL). The combined organic layers were washed with brine (3×50 mL), dried over anhydrous Na₂SO₄. After filtration, the filtrate was concentrated under reduced pressure. The crude product was purified by Prep-HPLC to afford 3-(5-(4-fluoro-6-methylisoindoline-2-carbonyl)-1-oxoisoindolin-2-yl)piperidine-2,6-dione (36 mg, 6.58%) as an off-white solid. LC-MS (ES, m/z)=422.2 [M+1]⁺. ¹H NMR (400 MHz, DMSO-d₆) δ 11.03 (s, 1H), 7.93-7.65 (m, 3H), 7.17-6.86 (m, 2H), 5.16 (dd, J=13.3, 5.1 Hz, 1H), 4.98-4.72 (m, 4H), 4.54 (dd, J=17.6, 4.0 Hz, 1H), 4.41 (dd, J=17.5, 3.2 Hz, 1H), 2.93 (ddd, J=18.0, 13.5, 5.4 Hz, 1H), 2.75-2.57 (m, 1H), 2.47-2.39 (m, 1H), 2.32 (d, J=14.8 Hz, 3H), 2.09-1.97 (m, 1H).

### Example 156

### Synthesis of 3-(5-(4-fluoro-6-methylisoindoline-2-carbonyl)-1-oxoisoindolin-2-yl)piperidine-2,6-dione

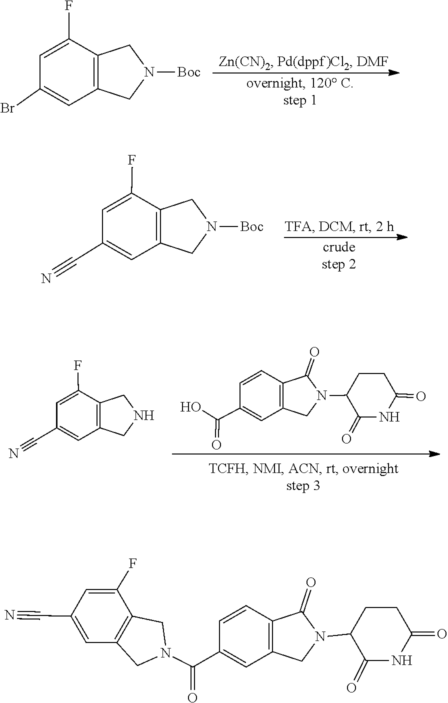 [SMILES: CC(C)(C)OC(=O)N1Cc2cc(Br)cc(F)c2C1 ; CC(C)(C)OC(=O)N1Cc2cc(C#N)cc(F)c2C1 ; N#Cc1cc(F)c2c(c1)CNC2 ; O=C1CCC(N2Cc3cc(C(=O)O)ccc3C2=O)C(=O)N1 ; N#Cc1cc(F)c2c(c1)CN(C(=O)c1ccc3c(c1)CN(C1CCC(=O)NC1=O)C3=O)C2]

Step 1. Preparation of tert-butyl 6-cyano-4-fluoroisoindoline-2-carboxylate. A mixture of tert-butyl 6-bromo-4-fluoro-1,3-dihydroisoindole-2-carboxylate (500 mg, 1.581 mmol, 1 equiv), Zn(CN)₂ (649.9 mg, 5.534 mmol, 3.5 equiv) and Pd(dppf)Cl₂ (115.7 mg, 0.158 mmol, 0.1 equiv) in DMF (7.5 mL) was stirred for overnight at 120° C. under nitrogen atmosphere. The resulting mixture was diluted with water and extracted with EtOAc (3×50 mL). The combined organic layers were washed with brine (3×50 mL), dried over anhydrous Na₂SO₄. After filtration, the filtrate was concentrated under reduced pressure. The residue was purified by reversed-phase flash chromatography to afford tert-butyl 6-cyano-4-fluoroisoindoline-2-carboxylate (260 mg, 63%) as a yellow solid. LC-MS (ES, m/z)=248.1 [M-t-Bu+ACN]⁺.

Step 2. Preparation of 7-fluoroisoindoline-5-carbonitrile. A mixture of tert-butyl 6-cyano-4-fluoro-1,3-dihydroisoindole-2-carboxylate (260 mg, 0.991 mmol, 1 equiv) in DCM (3 mL) was added TFA (0.6 mL) dropwise at 0° C. under air atmosphere. The resulting mixture was stirred for 2 h at room temperature under the air atmosphere. The resulting mixture was concentrated under vacuum to afford 7-fluoroisoindoline-5-carbonitrile (280 mg, crude) as a brown solid. The crude was used for the next step without further purification. LC-MS (ES, m/z)=163.1 [M+1]⁺.

Step 3. Preparation of 2-(2-(2,6-dioxopiperidin-3-yl)-1-oxoisoindoline-5-carbonyl)-7-fluoroisoindoline-5-carbonitrile. A mixture of tert-butyl 6-cyano-4-fluoro-1,3-dihydroisoindole-2-carboxylate (250 mg, 0.953 mmol, 1 equiv) and 2-(2,6-dioxopiperidin-3-yl)-1-oxo-3H-isoindole-5-carboxylic acid (357.2 mg, 1.239 mmol, 1.3 equiv) in acetonitrile (4 mL) was added NMI (313.0 mg, 3.812 mmol, 4 equiv) and TCFH (534.9 mg, 1.906 mmol, 2 equiv) in portions at room temperature. The resulting mixture was stirred overnight at room temperature under air atmosphere. The resulting mixture was diluted with water and extracted with EtOAc (3×50 mL). The combined organic layers were washed with brine (3×20 mL), dried over anhydrous Na₂SO₄. After filtration, the filtrate was concentrated under reduced pressure. The crude product (150 mg) was purified by Prep-HPLC to afford 2-(2-(2,6-dioxopiperidin-3-yl)-1-oxoisoindoline-5-carbonyl)-7-fluoroisoindoline-5-carbonitrile (67 mg, 16%) as an off-white solid. LC-MS (ES, m/z)=433.1 [M+1]⁺. ¹H NMR (400 MHz, DMSO-d₆) δ 11.03 (s, 1H), 7.95-7.78 (m, 3H), 7.73 (dd, J=8.0, 4.3 Hz, 1H), 5.17 (dd, J=13.3, 5.1 Hz, 1H), 5.11-4.79 (m, 4H), 4.54 (dd, J=17.6, 4.0 Hz, 1H), 4.41 (dd, J=17.6, 3.7 Hz, 1H), 2.94 (ddd, J=18.1, 13.5, 5.4 Hz, 1H), 2.62 (d, J=17.6 Hz, 1H), 2.49-2.35 (m, 1H), 2.04 (dt, J=11.5, 5.3 Hz, 1H).

### Example 157

### Synthesis of 3-(5-(7,7-difluoro-2-azaspiro[3.5]nonane-2-carbonyl)-1-oxoisoindolin-2-yl)piperidine-2,6-dione

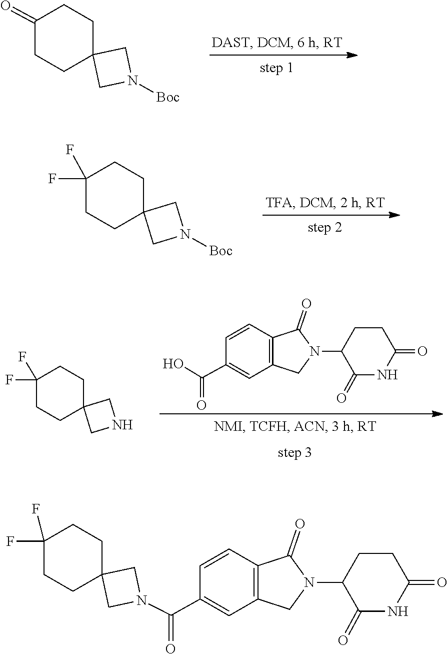 [SMILES: CC(C)(C)OC(=O)N1CC2(CCC(=O)CC2)C1 ; CC(C)(C)OC(=O)N1CC2(CCC(F)(F)CC2)C1 ; FC1(F)CCC2(CC1)CNC2 ; O=C1CCC(N2Cc3cc(C(=O)O)ccc3C2=O)C(=O)N1 ; O=C1CCC(N2Cc3cc(C(=O)N4CC5(CCC(F)(F)CC5)C4)ccc3C2=O)C(=O)N1]

Step 1. Preparation of tert-butyl 7,7-difluoro-2-azaspiro[3.5]nonane-2-carboxylate. To a stirred solution of tert-butyl 7-oxo-2-azaspiro[3.5]nonane-2-carboxylate (500 mg, 2.089 mmol, 1 equiv) in DCM (6 mL) was added DAST (1.68 g, 10.445 mmol, 5 equiv) dropwise at 0° C. under nitrogen atmosphere. The resulting mixture was stirred for 6 h at room temperature under nitrogen atmosphere. The reaction was quenched by the addition of sat. NaHCO₃ (aq.) (20 mL) at 0° C. The resulting mixture was diluted with water (200 mL). The resulting mixture was extracted with DCM (3×150 mL). The combined organic layers were washed with brine (3×150 mL), dried over anhydrous Na₂SO₄. After filtration, the filtrate was concentrated under reduced pressure to afford tert-butyl 7,7-difluoro-2-azaspiro[3.5]nonane-2-carboxylate (600 mg, crude) as a brown oil. LC-MS (ES, m/z)=247.15 [M-t-Bu+ACN]⁺.

Step 2. Preparation of 7,7-difluoro-2-azaspiro[3.5]nonane. To a stirred solution of tert-butyl 7,7-difluoro-2-azaspiro[3.5]nonane-2-carboxylate (600 mg, 2.296 mmol, 1 equiv) in DCM (5 mL) was added TFA (1 mL) dropwise at 0° C. The resulting mixture was stirred for 2 h at room temperature. The resulting mixture was diluted with water (100 mL). The resulting mixture was extracted with DCM (3×150 mL). The combined organic layers were washed with brine (3×150 mL), dried over anhydrous Na₂SO₄. After filtration, the filtrate was concentrated under reduced pressure to afford 7,7-difluoro-2-azaspiro[3.5]nonane (700 mg, crude) as a brown oil. LC-MS (ES, m/z)=162.15 [M+1]⁺.

Step 3. Preparation of 3-(5-(7,7-difluoro-2-azaspiro[3.5]nonane-2-carbonyl)-1-oxoisoindolin-2-yl)piperidine-2,6-dione. To a stirred solution of 7,7-difluoro-2-azaspiro[3.5]nonane (100 mg, 0.620 mmol, 1 equiv), 2-(2,6-dioxopiperidin-3-yl)-1-oxo-3H-isoindole-5-carboxylic acid (178.8 mg, 0.620 mmol, 1 equiv) and NMI (203.7 mg, 2.480 mmol, 4 equiv) in ACN (3 mL) was added TCFH (261.1 mg, 0.930 mmol, 1.5 equiv) in portions at room temperature. The resulting mixture was stirred for 3 h at room temperature. The resulting mixture was diluted with water and extracted with EtOAc (3×100 mL). The combined organic layers were washed with brine (2×100 mL), dried over anhydrous Na₂SO₄. After filtration, the filtrate was concentrated under reduced pressure. The crude product (80 mg) was purified by Prep-HPLC to afford 3-(5-(7,7-difluoro-2-azaspiro[3.5]nonane-2-carbonyl)-1-oxoisoindolin-2-yl)piperidine-2,6-dione (29 mg, 11%) as an off-white solid. LC-MS (ES, m/z)=432.3 [M+1]⁺.

¹H NMR (400 MHz, DMSO-d₆) δ 11.01 (s, 1H), 7.87 (d, J=1.3 Hz, 1H), 7.77 (qd, J=7.9, 1.1 Hz, 2H), 5.14 (dd, J=13.3, 5.1 Hz, 1H), 4.57-4.30 (m, 2H), 4.06 (s, 2H), 3.82 (s, 2H), 2.92 (ddd, J=17.2, 13.6, 5.4 Hz, 1H), 2.68-2.57 (m, 1H), 2.46-2.29 (m, 1H), 2.10-1.98 (m, 1H), 1.98-1.56 (m, 8H).

### Example 158

### Synthesis of 3-(6-(4-chloroisoindoline-2-carbonyl)benzo[d]oxazol-2-yl)piperidine-2,6-dione

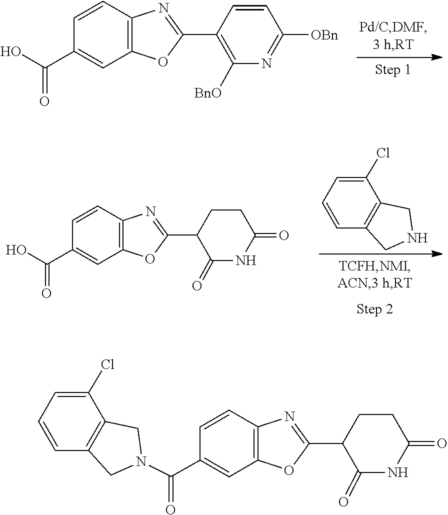 [SMILES: O=C(O)c1ccc2nc(-c3ccc(OCc4ccccc4)nc3OCc3ccccc3)oc2c1 ; O=C1CCC(c2nc3ccc(C(=O)O)cc3o2)C(=O)N1 ; Clc1cccc2c1CNC2 ; O=C1CCC(c2nc3ccc(C(=O)N4Cc5cccc(Cl)c5C4)cc3o2)C(=O)N1]

Step 1. Preparation of 2-(2,6-dioxopiperidin-3-yl)benzo[d]oxazole-6-carboxylic acid. A solution of 2-[2,6-bis(benzyloxy)pyridin-3-yl]-1,3-benzoxazole-6-carboxylic acid (900 mg, 1.989 mmol, 1 equiv) and Pd/C (899.6 mg, 10% on carbon (wetted with ca.55% water) in DMF (10 mL) was stirred at room temperature for 3 h under hydrogen atmosphere. The resulting mixture was filtered, and the filter cake was washed with DCM (3×20 mL). The filtrate was concentrated under reduced pressure. The residue was purified by reversed-phase flash chromatography to afford 2-(2,6-dioxopiperidin-3-yl)benzo[d]oxazole-6-carboxylic acid (350 mg, 58%) as an off-white solid. LC-MS (ES, m/z)=273.05 [M−1]⁻.

Step 2. Preparation of 3-(6-(4-chloroisoindoline-2-carbonyl)benzo[d]oxazol-2-yl)piperidine-2,6-dione. A solution of 2-(2,6-dioxopiperidin-3-yl)benzo[d]oxazole-6-carboxylic acid (350 mg, 1.276 mmol, 1 equiv), NMI (419.1 mg, 5.104 mmol, 4 equiv) and 4-chloro-2,3-dihydro-1H-isoindole (235.2 mg, 1.531 mmol, 1.2 equiv) in ACN (5 mL) was stirred for 3 min at room temperature. To the above mixture was added TCFH (537.1 mg, 1.914 mmol, 1.5 equiv) in portions at room temperature. The resulting mixture was stirred for additional 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to give the crude product (70 mg), which was further purified by Prep-HPLC to afford 3-(6-(4-chloroisoindoline-2-carbonyl)benzo[d]oxazol-2-yl)piperidine-2,6-dione (41 mg, 8%) as an off-white solid. LC-MS (ES, m/z)=410.10 [M+1]⁺. ¹H NMR (400 MHz, DMSO-d₆) δ 11.16 (s, 1H), 8.07 (t, J=1.9 Hz, 1H), 7.86 (dd, J=8.2, 3.0 Hz, 1H), 7.65 (ddd, J=8.2, 6.6, 1.5 Hz, 1H), 7.44-7.21 (m, 3H), 5.09-4.67 (m, 4H), 4.57 (dd, J=11.3, 5.1 Hz, 1H), 2.81-2.58 (m, 2H), 2.49-2.40 (m, 1H), 2.40-2.30 (m, 1H).

### Examples 159 and 160

### Synthesis of 3-(6-((R)-7-fluoro-1-methylisoindoline-2-carbonyl)benzo[d]oxazol-2-yl)piperidine-2,6-dione and 3-(6-((R)-7-fluoro-1-methylisoindoline-2-carbonyl)benzo[d]oxazol-2-yl)piperidine-2,6-dione

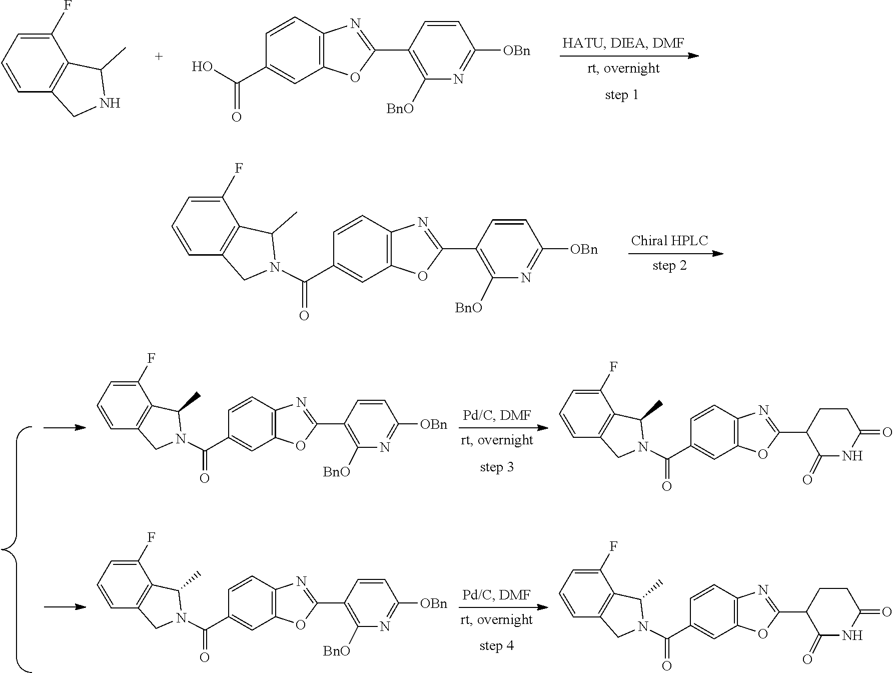

Step 1. Preparation of (2-(2,6-bis(benzyloxy)pyridin-3-yl)benzo[d]oxazol-6-yl)(7-fluoro-1-methylisoindolin-2-yl)methanone. To a stirred mixture of 7-fluoro-1-methyl-2,3-dihydro-1H-isoindole (268.6 mg, 1.777 mmol, 2.01 equiv) and 2-[2,6-bis(benzyloxy)pyridin-3-yl]-1,3-benzoxazole-6-carboxylic acid (400 mg, 0.884 mmol, 1.00 equiv) in ACN (8 mL) was added NMI (363 mg, 4.421 mmol, 5.00 equiv) at room temperature under air atmosphere. To the above mixture was added TCFH (372.1 mg, 1.326 mmol, 1.50 equiv) in portions over 1 min at room temperature. The resulting mixture was stirred for an additional 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(7-fluoro-1-methylisoindolin-2-yl)methanone (300 mg, 58%) as a brownish yellow solid. LC-MS (ES, m/z)=586.15 [M+1]⁺.

Step 2. Preparation of (R)-(2-(2,6-bis(benzyloxy)pyridin-3-yl)benzo[d]oxazol-6-yl)(7-fluoro-1-methylisoindolin-2-yl)methanone and (S)-(2-(2,6-bis(benzyloxy)pyridin-3-yl)benzo[d]oxazol-6-yl)(7-fluoro-1-methylisoindolin-2-yl)methanone. The product (2-(2,6-bis(benzyloxy)pyridin-3-yl)benzo[d]oxazol-6-yl)(7-fluoro-1-methylisoindolin-2-yl)methanone (400 mg, 0.683 mmol, 1 equiv) was separated by Chiral HPLC with the following conditions: Column: (S, S)-Whelk-O 1 5 μm Kromasil, 3*25 cm, 5 μm; Mobile Phase A: Hex (10 mM NH₃-MeOH), Mobile Phase B: EtOH:ACN=5:1; Flow rate: 40 mL/min; Gradient: isocratic 40; Wave Length: 210/328 nm; RT1 (min): 14.2; RT2 (min): 16.88; Sample Solvent: EtOH:DCM=2:1; Injection Volume: 1 mL; Number Of Runs: 20 to afford (R)-(2-(2,6-bis(benzyloxy)pyridin-3-yl)benzo[d]oxazol-6-yl)(7-fluoro-1-methylisoindolin-2-yl)methanone (170 mg, 43%, RT: 1.285 min) as an off-white solid and (S)-(2-(2,6-bis(benzyloxy)pyridin-3-yl)benzo[d]oxazol-6-yl)(7-fluoro-1-methylisoindolin-2-yl)methanone (160 mg, 40%, RT: 1.537 min) as an off-white solid. LC-MS (ES, m/z)=586.15 [M+1]⁺.

Step 3. Preparation of 3-(6-((R)-7-fluoro-1-methylisoindoline-2-carbonyl)benzo[d]oxazol-2-yl)piperidine-2,6-dione. To a solution of (R)-(2-(2,6-bis(benzyloxy)pyridin-3-yl)benzo[d]oxazol-6-yl)(7-fluoro-1-methylisoindolin-2-yl)methanone (150 mg, 0.256 mmol, 1 equiv) in DMF (3.21 mL) was added Pd/C (70 mg, Palladium 10% on Carbon (wetted with ca. 55% Water)) under nitrogen atmosphere. The mixture was hydrogenated at room temperature for 2 h under hydrogen atmosphere using a hydrogen balloon, filtered through a Celite pad and concentrated under reduced pressure. The residue was purified by reversed-phase flash chromatography to afford 3-(6-((R)-7-fluoro-1-methylisoindoline-2-carbonyl)benzo[d]oxazol-2-yl)piperidine-2,6-dione (87 mg, 83%) as an off-white solid. LC-MS (ES, m/z)=408.15 [M+1]⁺. ¹H NMR (400 MHz, DMSO-d₆) δ 11.17 (s, 1H), 8.07 (t, J=6.6 Hz, 1H), 7.85 (d, J=8.1 Hz, 1H), 7.63 (d, J=8.1 Hz, 1H), 7.42-7.25 (m, 1H), 7.18-7.06 (m, 2H), 5.64 (q, J=6.4 Hz, 1H), 5.11 (dd, J=15.0, 8.6 Hz, 1H), 4.62-4.53 (m, 2H), 2.81-2.59 (m, 2H), 2.49-2.38 (m, 1H), 2.35 (dq, J=13.5, 4.9 Hz, 1H), 1.59 (d, J=6.4 Hz, 2H), 1.07 (d, J=6.3 Hz, 1H).

Step 4. Preparation of 3-(6-((S)-7-fluoro-1-methylisoindoline-2-carbonyl)benzo[d]oxazol-2-yl)piperidine-2,6-dione. To a solution of (S)-(2-(2,6-bis(benzyloxy)pyridin-3-yl)benzo[d]oxazol-6-yl)(7-fluoro-1-methylisoindolin-2-yl)methanone (140 mg, 0.239 mmol, 1 equiv) in DMF (3 mL) was added Pd/C (70 mg, Palladium 10% on Carbon (wetted with ca. 55% Water)) under nitrogen atmosphere. The mixture was hydrogenated at room temperature for 2 h under hydrogen atmosphere using a hydrogen balloon, filtered through a Celite pad and concentrated under reduced pressure. The residue was purified by reversed-phase flash chromatography to afford 3-(6-((S)-7-fluoro-1-methylisoindoline-2-carbonyl)benzo[d]oxazol-2-yl)piperidine-2,6-dione (66 mg, 67%) as an off-white solid. LC-MS (ES, m/z)=408.1 [M+1]⁺.

¹H NMR (400 MHz, DMSO-d₆) δ 11.17 (s, 1H), 8.07 (t, J=6.8 Hz, 1H), 7.85 (d, J=8.0 Hz, 1H), 7.63 (d, J=8.1 Hz, 1H), 7.43-7.25 (m, 1H), 7.18-7.06 (m, 2H), 5.64 (q, J=6.5 Hz, 1H), 5.11 (dd, J=14.8, 8.6 Hz, 1H), 4.62-4.53 (m, 2H), 2.81-2.59 (m, 2H), 2.44 (11, J=11.2, 3.6 Hz, 1H), 2.35 (dt, J=13.1, 4.8 Hz, 1H), 1.59 (d, J=6.4 Hz, 2H), 1.07 (d, J=6.3 Hz, 1H).

### Example 161

### Synthesis of 3-(6-(4-methylisoindoline-2-carbonyl)benzo[d]oxazol-2-yl)piperidine-2,6-dione

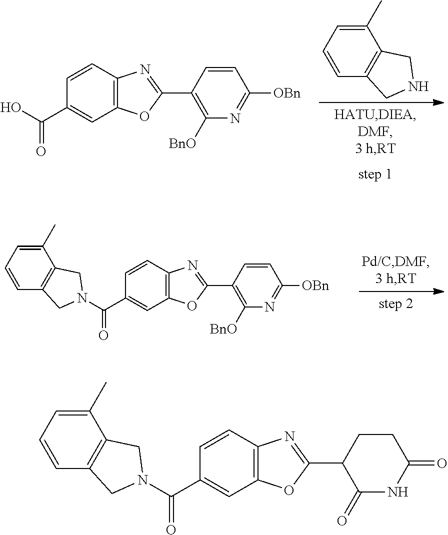

Step 1. Preparation of (2-(2,6-bis(benzyloxy)pyridin-3-yl)benzo[d]oxazol-6-yl)(4-methylisoindolin-2-yl)methanone. A solution of 2-[2,6-bis(benzyloxy)pyridin-3-yl]-1,3-benzoxazole-6-carboxylic acid (90 mg, 0.199 mmol, 1 equiv), DIEA (77.13 mg, 0.597 mmol, 3 equiv) and 4-methyl-2,3-dihydro-1H-isoindole (79.5 mg, 0.597 mmol, 3 equiv) in DMF (2 mL) was stirred for 3 min at room temperature. To the above mixture was added HATU (113.5 mg, 0.298 mmol, 1.5 equiv) in portions at room temperature. The resulting mixture was stirred for additional 3 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(4-methylisoindolin-2-yl)methanone (77 mg, 68%) as an off-white solid. LC-MS (ES, m/z)=568.35 [M+1]⁺.

Step 2. Preparation of 3-(6-(4-methylisoindoline-2-carbonyl)benzo[d]oxazol-2-yl)piperidine-2,6-dione. A solution of (2-(2,6-bis(benzyloxy)pyridin-3-yl)benzo[d]oxazol-6-yl)(4-methylisoindolin-2-yl)methanone (60 mg, 0.106 mmol, 1 equiv) and Pd/C (168.7 mg, 0.159 mmol, 1.5 equiv) 10% on carbon (wetted with ca. 55% water) in DMF (1.5 mL) was stirred for 3 h at room temperature under hydrogen atmosphere. The precipitated solids were collected by filtration and washed with DCM (3×5 mL). The resulting mixture was concentrated under vacuum. The residue was purified by reversed-phase flash chromatography to afford 3-(6-(4-methylisoindoline-2-carbonyl)benzo[d]oxazol-2-yl)piperidine-2,6-dione (6.9 mg, 17%) as an off-white solid. LC-MS (ES, m/z)=390.1 [M+1]⁺. ¹H NMR (400 MHz, DMSO-d₆) δ 11.18 (s, 1H), 8.07 (s, 1H), 7.85 (dd, J=8.2, 3.0 Hz, 1H), 7.64 (t, J=8.1 Hz, 1H), 7.21 (dd, J=11.8, 5.9 Hz, 1H), 7.14 (d, J=20.0 Hz, 1H), 7.07 (t, J=8.8 Hz, 1H), 4.91 (s, 1H), 4.86-4.76 (m, 3H), 4.57 (dd, J=11.3, 5.0 Hz, 1H), 2.75 (ddd, J=17.0, 11.3, 5.4 Hz, 1H), 2.65 (dt, J=17.5, 4.6 Hz, 1H), 2.49-2.42 (m, 1H), 2.38-2.31 (m, 1H), 2.30 (s, 1H), 2.12 (s, 2H).

### Example 162

### Synthesis of 3-(6-(4-methoxyisoindoline-2-carbonyl)benzo[d]oxazol-2-yl)piperidine-2,6-dione

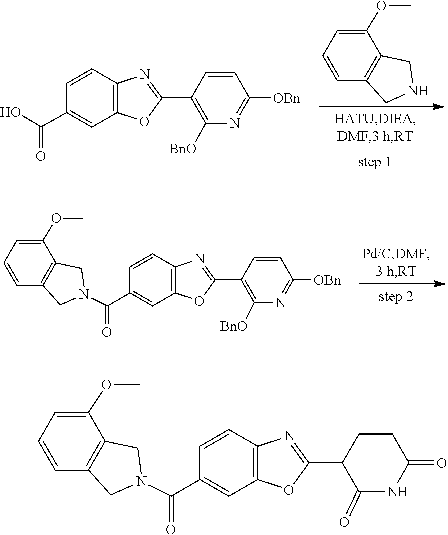 [SMILES: O=C(O)c1ccc2nc(-c3ccc(OCc4ccccc4)nc3OCc3ccccc3)oc2c1 ; COc1cccc2c1CNC2 ; COc1cccc2c1CN(C(=O)c1ccc3nc(-c4ccc(OCc5ccccc5)nc4OCc4ccccc4)oc3c1)C2 ; COc1cccc2c1CN(C(=O)c1ccc3nc(C4CCC(=O)NC4=O)oc3c1)C2]

Step 1. Preparation of (2-(2,6-bis(benzyloxy)pyridin-3-yl)benzo[d]oxazol-6-yl)(4-methoxyisoindolin-2-yl)methanone. To a stirred mixture of 2-[2,6-bis(benzyloxy)pyridin-3-yl]-1,3-benzoxazole-6-carboxylic acid (130 mg, 0.287 mmol, 1 equiv) and 4-methoxy-2,3-dihydro-1H-isoindole (42.8 mg, 0.287 mmol, 1 equiv) in ACN (3 mL) were added TCFH (120.9 mg, 0.430 mmol, 1.5 equiv) and NMI (94.3 mg, 1.148 mmol, 4 equiv) at room temperature. The resulting mixture was stirred for 3 h at room temperature. The resulting mixture was diluted with water (30 mL) and extracted with EtOAc (3×10 mL). The combined organic layers were washed with brine (2×20 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(4-methoxyisoindolin-2-yl)methanone (90 mg, 54%) as a white solid. LC-MS (ES, m/z)=584.0 [M+1]⁺.

Step 2. Preparation of 3-(6-(4-methoxyisoindoline-2-carbonyl)benzo[d]oxazol-2-yl)piperidine-2,6-dione. To a stirred mixture of (2-(2,6-bis(benzyloxy)pyridin-3-yl)benzo[d]oxazol-6-yl)(4-methoxyisoindolin-2-yl)methanone (90 mg, 0.154 mmol, 1 equiv) in DMF (1.5 mL) were added Pd/C (50 mg, Palladium 10% on Carbon (wetted with ca. 55% Water)) at room temperature. The resulting mixture was stirred overnight at room temperature under hydrogen atmosphere. The resulting mixture was filtered, the filter cake was washed with THF (3×5 mL). The filtrate was concentrated under reduced pressure. The crude product was purified by Prep-HPLC to afford 3-(6-(4-methoxyisoindoline-2-carbonyl)benzo[d]oxazol-2-yl)piperidine-2,6-dione (18 mg, 28%) as a white solid. LC-MS (ES, m/z)=406.05 [M+1]⁺. ¹H NMR (400 MHz, DMSO-d₆) δ 11.16 (s, 1H), 8.06 (s, 1H), 7.84 (dd, J=8.1, 1.7 Hz, 1H), 7.63 (dd, J=8.2, 1.5 Hz, 1H), 7.29 (dt, J=16.2, 7.9 Hz, 1H), 6.97-6.80 (m, 2H), 4.89 (s, 1H), 4.80 (d, J=6.0 Hz, 2H), 4.68 (s, 1H), 4.57 (dd, J=11.3, 5.1 Hz, 1H), 3.79 (d, J=42.9 Hz, 3H), 2.73-2.62 (m, 2H), 2.35 (d, J=10.9 Hz, 2H).

### Example 163

### Synthesis of 3-(5-(6,6-difluoro-2-azaspiro[3.4]octane-2-carbonyl)-1-oxoisoindolin-2-yl)piperidine-2,6-dione

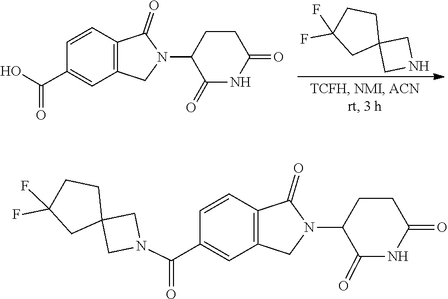

Step 1. Preparation of 3-(5-(6,6-difluoro-2-azaspiro[3.4]octane-2-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (50 mg, 0.173 mmol, 1 equiv) and 6,6-difluoro-2-azaspiro[3.4]octane (25.5 mg, 0.173 mmol, 1 equiv) in ACN (1.5 mL) were added TCFH (73.0 mg, 0.259 mmol, 1.5 equiv) and NMI (56.9 mg, 0.692 mmol, 4 equiv) at room temperature. The resulting mixture was stirred for 3 h at room temperature. The resulting mixture was diluted with water (20 mL) and extracted with EtOAc (3×10 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(5-(6,6-difluoro-2-azaspiro[3.4]octane-2-carbonyl)-1-oxoisoindolin-2-yl)piperidine-2,6-dione (33 mg, 45%) as a white solid. LC-MS (ES, m/z)=418.05 [M+1]⁺. ¹H NMR (400 MHz, DMSO-d₆) δ 11.02 (s, 1H), 7.86 (s, 1H), 7.77 (q, J=8.0 Hz, 2H), 5.14 (dd, J=13.4, 5.1 Hz, 1H), 4.51 (d, J 17.5 Hz, 1H), 4.38 (d, J=17.4 Hz, 1H), 4.28 (d, J=9.0 Hz, 1H), 4.22 (d, J=8.8 Hz, 1H), 4.04 (d, J=10.2 Hz, 1H), 3.98 (d, J=10.1 Hz, 1H), 2.96-2.85 (m, 1H), 2.64-2.57 (m, 1H), 2.42 (t, J=14.1 Hz, 3H), 2.18-2.00 (m, 5H).

### Example 164

### Synthesis of (2-(2,6-dioxopiperidin-3-yl)benzo[d]oxazol-5-yl)methyl (2-fluoro-5-(trifluoromethoxy)phenyl)carbamate

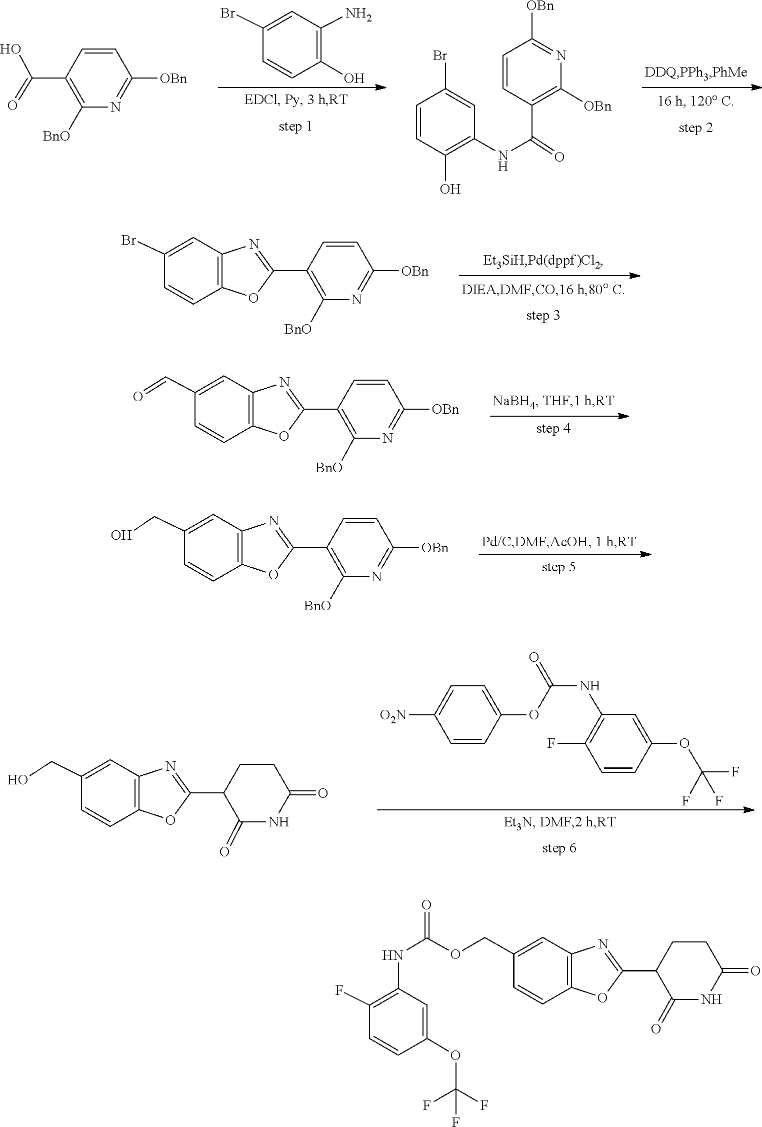

Step 1. Preparation of 2,6-bis(benzyloxy)-N-(5-bromo-2-hydroxyphenyl)nicotinamide. A solution of 2,6-bis(benzyloxy)pyridine-3-carboxylic acid (4.2 g, 12.524 mmol, 1 equiv), EDCI (3.6 g, 18.786 mmol, 1.5 equiv) and 2-amino-4-bromophenol (3.5 g, 18.786 mmol, 1.5 equiv) in pyridine (40 mL) was stirred for 3 h at room temperature. The resulting mixture was diluted with water (300 mL) and extracted with EtOAc (3×200 mL). The combined organic layers were washed with brine (2×200 mL), dried over anhydrous Na₂SO₄. After filtration, the filtrate was concentrated under reduced pressure. The residue was purified by reversed-phase flash chromatography to afford 2,6-bis(benzyloxy)-N-(5-bromo-2-hydroxyphenyl)nicotinamide (3 g, 47%) as an off-white solid. LC-MS (ES, m/z)=505.15 [M+1]⁺.

Step 2. Preparation of 2-(2,6-bis(benzyloxy)pyridin-3-yl)-5-bromobenzo[d]oxazole. A solution of 2,6-bis(benzyloxy)-N-(5-bromo-2-hydroxyphenyl)nicotinamide (3 g, 5.936 mmol, 1 equiv), PPh₃ (2.3 g, 8.904 mmol, 1.5 equiv) and DDQ (2.0 g, 8.904 mmol, 1.5 equiv) in methylbenzene (20 mL) was stirred for 16 h at 120° C. The resulting mixture was diluted with water (300 mL) and extracted with EtOAc (3×150 mL). The combined organic layers were washed with brine (2×100 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-bromobenzo[d]oxazole (1 g, 35%) as a light yellow solid. LC-MS (ES, m/z)=487.1 [M+1]⁺.

Step 3. Preparation of 2-(2,6-bis(benzyloxy)pyridin-3-yl)benzo[d]oxazole-5-carbaldehyde. A solution of 2-(2,6-bis(benzyloxy)pyridin-3-yl)-5-bromobenzo[d]oxazole (1 g, 2.052 mmol, 1 equiv), Pd(dppf)Cl₂ (150.1 mg, 0.205 mmol, 0.1 equiv), DIEA (1.3 g, 10.260 mmol, 5 equiv) and Et₃SiH (1.9 g, 16.416 mmol, 8 equiv) in DMF (10 mL) was stirred for 16 h at 80° C. under carbon monoxide atmosphere. The resulting mixture was diluted with water (100 mL) and extracted with EtOAc (3×50 mL). The combined organic layers were washed with brine (2×30 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)benzo[d]oxazole-5-carbaldehyde (800 mg, 89%) as an off-white solid. LC-MS (ES, m/z)=437.05 [M+1]⁺.

Step 4. Preparation of (2-(2,6-bis(benzyloxy)pyridin-3-yl)benzo[d]oxazol-5-yl)methanol. A solution of 2-(2,6-bis(benzyloxy)pyridin-3-yl)benzo[d]oxazole-5-carbaldehyde (1 g, 2.291 mmol, 1 equiv) in THF (5 mL) was stirred for 3 min at room temperature. To the above mixture was added NaBH₄ (173.4 mg, 4.582 mmol, 2 equiv) in portions at 0° C. The resulting mixture was stirred for an additional 1 h at room temperature. The reaction was quenched with Water at 0° C. and extracted with EtOAc (3×50 mL). The combined organic layers were washed with brine (2×30 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-5-yl)methanol (700 mg, 70%) as an off-white solid. LC-MS (ES, m/z)=439.2 [M+1]⁺.

Step 5. Preparation of 3-(5-(hydroxymethyl)benzo[d]oxazol-2-yl)piperidine-2,6-dione. A solution of (2-(2,6-bis(benzyloxy)pyridin-3-yl)benzo[d]oxazol-5-yl)methanol (700 mg, 1.596 mmol, 1 equiv), AcOH (479.3 mg, 7.980 mmol, 5 equiv) and Pd/C (10% on carbon (wetted with ca.55% water), 1.70 g) in DMF (5 mL) was stirred for 1 h at room temperature under hydrogen atmosphere. The resulting mixture was filtered, the filter cake was washed with THF (3×5 mL). The filtrate was concentrated under reduced pressure. The residue was purified by reversed-phase flash chromatography to afford 3-(5-(hydroxymethyl)benzo[d]oxazol-2-yl)piperidine-2,6-dione (350 mg, 84%) as an off-white solid. LC-MS (ES, m/z)=261.05 [M+1]⁺.

Step 6. Preparation of (2-(2,6-dioxopiperidin-3-yl)benzo[d]oxazol-5-yl)methyl (2-fluoro-5-(trifluoromethoxy)phenyl)carbamate. A solution of 3-(5-(hydroxymethyl)benzo[d]oxazol-2-yl)piperidine-2,6-dione (150 mg, 0.576 mmol, 1 equiv) and Et₃N (349.9 mg, 3.456 mmol, 6 equiv) in DMF (2 mL) was stirred for 5 min at 0° C. To the above mixture was added 4-nitrophenyl N-[2-fluoro-5-(trifluoromethoxy)phenyl]carbamate (415.2 mg, 1.152 mmol, 2 equiv) at room temperature. The resulting mixture was stirred for an additional 2 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to give crude product (90 mg), which was further purified by Prep-HPLC to afford (2-(2,6-dioxopiperidin-3-yl)benzo[d]oxazol-5-yl)methyl (2-fluoro-5-(trifluoromethoxy)phenyl)carbamate (35 mg, 13%) as an off-white solid. LC-MS (ES, m/z)=482.2 [M+1]⁺. ¹H NMR (400 MHz, DMSO-d₆) δ 11.15 (s, 1H), 9.88 (s, 1H), 7.84 (dd, J=7.1, 2.4 Hz, 2H), 7.77 (d, J=8.4 Hz, 1H), 7.51 (dd, J=8.5, 1.7 Hz, 1H), 7.38 (dd, J=10.5, 9.0 Hz, 1H), 7.18-7.08 (m, 1H), 5.31 (s, 2H), 4.53 (dd, J=11.2, 5.1 Hz, 1H), 2.79-2.57 (m, 2H), 2.45-2.22 (m, 2H).

### Example 165

### Synthesis of 3-(1-oxo-5-(6-(trifluoromethoxy)-2-azaspiro[3.3]heptane-2-carbonyl)isoindolin-2-yl)piperidine-2,6-dione

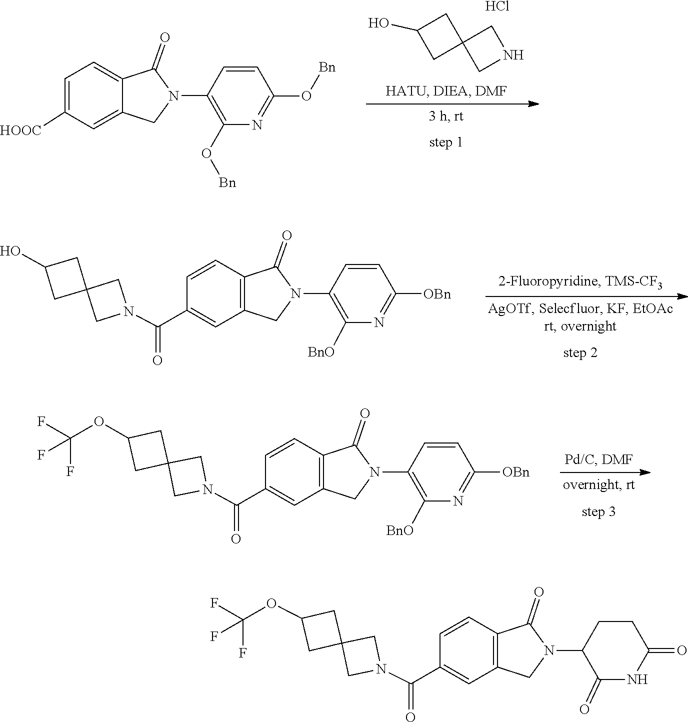 [SMILES: Cl ; O=C(O)c1ccc2c(c1)CN(c1ccc(OCc3ccccc3)nc1OCc1ccccc1)C2=O ; OC1CC2(CNC2)C1 ; O=C(c1ccc2c(c1)CN(c1ccc(OCc3ccccc3)nc1OCc1ccccc1)C2=O)N1CC2(CC(O)C2)C1 ; O=C(c1ccc2c(c1)CN(c1ccc(OCc3ccccc3)nc1OCc1ccccc1)C2=O)N1CC2(CC(OC(F)(F)F)C2)C1 ; O=C1CCC(N2Cc3cc(C(=O)N4CC5(CC(OC(F)(F)F)C5)C4)ccc3C2=O)C(=O)N1]

Step 1. Preparation of 2-(2,6-bis(benzyloxy)pyridin-3-yl)-5-(6-hydroxy-2-azaspiro[3.3]heptane-2-carbonyl)isoindolin-1-one. To a stirred mixture of 2-azaspiro[3.3]heptan-6-ol (242.5 mg, 2.144 mmol, 1 equiv) and 2-[2,6-bis(benzyloxy)pyridin-3-yl]-1-oxo-3H-isoindole-5-carboxylic acid (1 g, 2.144 mmol, 1.00 equiv) in DMF (10 mL) were added HATU (1.22 g, 3.216 mmol, 1.5 equiv) and DIEA (831.1 mg, 6.432 mmol, 3 equiv) at room temperature. The resulting mixture was stirred for 3 h at room temperature. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(6-hydroxy-2-azaspiro[3.3]heptane-2-carbonyl)isoindolin-1-one (1 g, 83%) as a white solid. LC-MS (ES, m/z)=562.0 [M+1]⁺.

Step 2. Preparation of 2-(2,6-bis(benzyloxy)pyridin-3-yl)-5-(6-(trifluoromethoxy)-2-azaspiro[3.3]heptane-2-carbonyl)isoindolin-1-one. To a stirred mixture of 2-(2,6-bis(benzyloxy)pyridin-3-yl)-5-(6-hydroxy-2-azaspiro[3.3]heptane-2-carbonyl)isoindolin-1-one (500 mg, 0.890 mmol, 1 equiv), silver trifluoromethanesulfonate (914.9 mg, 3.560 mmol, 4 equiv), 4-(chloromethyl)-1-fluoro-1,4-diazabicyclo[2.2.2]octane-1,4-diium (473.1 mg, 1.335 mmol, 1.5 equiv) and KF (206.8 mg, 3.560 mmol, 4 equiv) in EtOAc (10 mL) were added trimethyl(trifluoromethyl)silane (316.4 mg, 2.225 mmol, 2.5 equiv) and 2-Fluoropyridine (345.7 mg, 3.560 mmol, 4 equiv) at room temperature under nitrogen atmosphere. The resulting mixture was stirred overnight at room temperature under nitrogen atmosphere. The resulting mixture was filtered, the filter cake was washed with EtOAc (3×20 mL). The filtrate was concentrated under reduced pressure. The residue was purified by reversed-phase flash chromatography to 2-(2,6-bis(benzyloxy)pyridin-3-yl)-5-(6-(trifluoromethoxy)-2-azaspiro[3.3]heptane-2-carbonyl)isoindolin-1-one (200 mg, 36%) as a yellow solid. LC-MS (ES, m/z)=630.0 [M+1]⁺.

Step 3. Preparation of 3-(1-oxo-5-(6-(trifluoromethoxy)-2-azaspiro[3.3]heptane-2-carbonyl)isoindolin-2-yl)piperidine-2,6-dione. To a stirred mixture of 2-(2,6-bis(benzyloxy)pyridin-3-yl)-5-(6-(trifluoromethoxy)-2-azaspiro[3.3]heptane-2-carbonyl)isoindolin-1-one (300 mg, 0.476 mmol, 1 equiv) in DMF (3 mL) were added Pd/C (150 mg, Palladium 10% on Carbon (wetted with ca. 55% Water)) at room temperature. The resulting mixture was stirred overnight at room temperature under hydrogen atmosphere. The resulting mixture was filtered, the filter cake was washed with EtOAc (3×10 mL). The filtrate was concentrated under reduced pressure. The residue was purified by reversed-phase flash chromatography to afford 3-(1-oxo-5-(6-(trifluoromethoxy)-2-azaspiro[3.3]heptane-2-carbonyl)isoindolin-2-yl)piperidine-2,6-dione (68 mg, 32%) as a white solid. LC-MS (ES, m/z)=452.1 [M+1]⁺. ¹H NMR (400 MHz, DMSO-d₆) δ 11.01 (s, 1H), 7.84 (s, 1H), 7.78 (d, J=7.9 Hz, 1H), 7.72 (d, J=7.9 Hz, 1H), 5.14 (dd, J=13.3, 5.1 Hz, 1H), 4.76 (dt, J=28.5, 7.2 Hz, 1H), 4.50 (d, J=17.6 Hz, 1H), 4.42-4.32 (m, 2H), 4.30 (s, 1H), 4.09 (d, J=14.7 Hz, 2H), 2.92 (ddd, J=17.1, 13.6, 5.4 Hz, 1H), 2.68-2.56 (m, 3H), 2.41 (dq, J=14.7, 8.7 Hz, 3H), 2.02 (ddd, J=10.5, 5.5, 3.3 Hz, 1H).

### Examples 166 and 167

### Synthesis of 3-(6-((R)-6-fluoro-3-methylindoline-1-carbonyl)benzo[d]oxazol-2-yl)piperidine-2,6-dione and 3-(6-((S)-6-fluoro-3-methylindoline-1-carbonyl)benzo[d]oxazol-2-yl)piperidine-2,6-dione

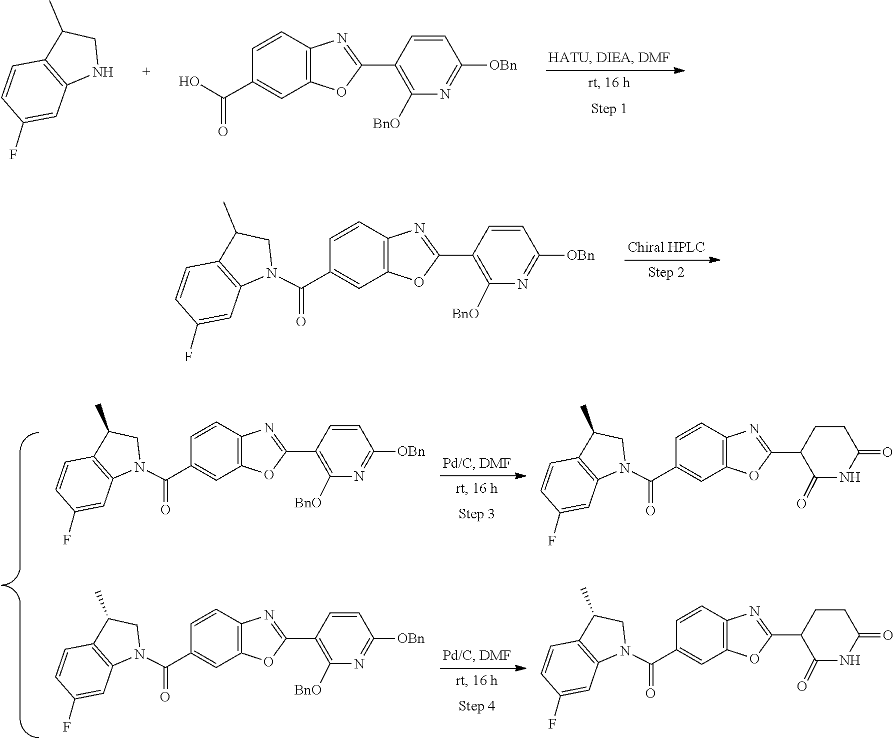

Step 1. Preparation of (2-(2,6-bis(benzyloxy)pyridin-3-yl)benzo[d]oxazol-6-yl)(6-fluoro-3-methylindolin-1-yl)methanone. To a stirred solution of 2-[2,6-bis(benzyloxy)pyridin-3-yl]-1,3-benzoxazole-6-carboxylic acid (620 mg, 1.370 mmol, 1.00 equiv) and DIEA (531.31 mg, 4.110 mmol, 3.0 equiv) in DMF (10 mL) were added 6-fluoro-3-methyl-2,3-dihydro-1H-indole (540 mg, 3.572 mmol, 2.61 equiv) and HATU (625.23 mg, 1.644 mmol, 1.2 equiv) in portions at room temperature. The resulting mixture was stirred overnight at room temperature. The resulting mixture was diluted with diluted with water and extracted with EtOAc (3×100 mL). The combined organic layers were washed with brine (2×100 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(6-fluoro-3-methylindolin-1-yl)methanone (600 mg, 75%) as a white solid. LC-MS (ES, m/z)=586.30 [M+1]⁺.

Step 2. Preparation of (R)-(2-(2,6-bis(benzyloxy)pyridin-3-yl)benzo[d]oxazol-6-yl)(6-fluoro-3-methylindolin-1-yl)methanone and (S)-(2-(2,6-bis(benzyloxy)pyridin-3-yl)benzo[d]oxazol-6-yl)(6-fluoro-3-methylindolin-1-yl)methanone. (2-(2,6-bis(benzyloxy)pyridin-3-yl)benzo[d]oxazol-6-yl)(6-fluoro-3-methylindolin-1-yl)methanone (600 mg, 1.025 mmol) was purified by Chiral HPLC with the following conditions (Column: CHIRAL ART Cellulose-SB, 3*25 cm, 5 μm; Mobile Phase A: CO₂, Mobile Phase B: MeOH:DCM=1:1 (20 mM NH₃); Flow rate: 100 mL/min; Gradient: isocratic 40% B; Column Temperature (° C.): 35; Back Pressure (bar): 100; Wave Length: 326/256 nm; RT1 (min): 3.57; RT2 (min): 4.98; Sample Solvent: MeOH:DCM=1:1-HPLC; Injection Volume: 3.5 mL; Number Of Runs: 8) to afford (R)-(2-(2,6-bis(benzyloxy)pyridin-3-yl)benzo[d]oxazol-6-yl)(6-fluoro-3-methylindolin-1-yl)methanone (162 mg, RT: 2.612 min) as a white solid and (S)-(2-(2,6-bis(benzyloxy)pyridin-3-yl)benzo[d]oxazol-6-yl)(6-fluoro-3-methylindolin-1-yl)methanone (210 mg, RT: 1.898 min) as a white solid. LC-MS (ES, m/z)=586.30 [M+1]⁺.

Step 3. Preparation of 3-(6-((R)-6-fluoro-3-methylindoline-1-carbonyl)benzo[d]oxazol-2-yl)piperidine-2,6-dione. To a stirred solution of (R)-(2-(2,6-bis(benzyloxy)pyridin-3-yl)benzo[d]oxazol-6-yl)(6-fluoro-3-methylindolin-1-yl)methanone (152 mg, 0.260 mmol, 1 equiv) in DMF (4.0 mL) was added Pd/C (138.11 mg, Palladium 10% on Carbon (wetted with ca. 55% Water)) at room temperature. The resulting mixture was stirred overnight at room temperature under hydrogen atmosphere. The precipitated solids were collected by filtration and washed with DMF (2×2 mL). The crude product (120 mg) was purified by Prep-HPLC to afford 3-(6-((R)-6-fluoro-3-methylindoline-1-carbonyl)benzo[d]oxazol-2-yl)piperidine-2,6-dione (79 mg, 75%) as a white solid. LC-MS (ES, m/z)=408.20 [M+1]. ¹H NMR (400 MHz, DMSO-d₆) δ 8.10 (s, 1H), 7.84-7.76 (m, 2H), 7.58 (d, J=8.1 Hz, 1H), 7.12 (ddd, J=8.4, 5.5, 1.1 Hz, 1H), 6.78 (t, J=8.6 Hz, 1H), 4.28 (dd, J=16.4, 10.5 Hz, 2H), 3.67 (t, J=8.9 Hz, 1H), 3.42 (h, J=7.2 Hz, 1H), 2.96 (d, J=17.1 Hz, 1H), 2.80-2.62 (m, 2H), 2.54-2.46 (m, 1H), 1.31 (d, J=6.8 Hz, 3H).

Step 4. Preparation of 3-(6-((S)-6-fluoro-3-methylindoline-1-carbonyl)benzo[d]oxazol-2-yl)piperidine-2,6-dione. To a stirred solution of (S)-(2-(2,6-bis(benzyloxy)pyridin-3-yl)benzo[d]oxazol-6-yl)(6-fluoro-3-methylindolin-1-yl)methanone (200 mg, 0.342 mmol, 1 equiv) in DMF (4 mL) was added Pd/C (181.72 mg, Palladium 10% on Carbon (wetted with ca. 55% Water)) at room temperature. The resulting mixture was stirred overnight at room temperature under hydrogen atmosphere. The precipitated solids were collected by filtration and washed with DMF (2×2 mL). The crude product was purified by Prep-HPLC to afford 3-(6-((S)-6-fluoro-3-methylindoline-1-carbonyl)benzo[d]oxazol-2-yl)piperidine-2,6-dione (59 mg, 43%) as a white solid. LC-MS (ES, m/z)=408.20 [M+1]⁺. ¹H NMR (400 MHz, DMSO-d₆) δ 8.17 (s, 1H), 7.83-7.77 (m, 2H), 7.63-7.54 (m, 1H), 7.12 (ddd, J=8.3, 5.5, 1.1 Hz, 1H), 6.78 (t, J 7.9 Hz, 1H), 4.34-4.19 (m, 2H), 3.67 (t, J=9.0 Hz, 1H), 3.42 (h, J=7.2 Hz, 1H), 3.03-2.89 (m, 1H), 2.71 (dddd, J=36.5, 17.7, 8.9, 4.8 Hz, 2H), 2.49 (dq, J=13.3, 5.0 Hz, 1H), 1.31 (d, J 6.8 Hz, 3H).

### Example 168

### Synthesis of 3-(5-(6-fluoro-6-(4-fluorophenyl)-2-azaspiro[3.3]heptane-2-carbonyl)-1-oxoisoindolin-2-yl)piperidine-2,6-dione

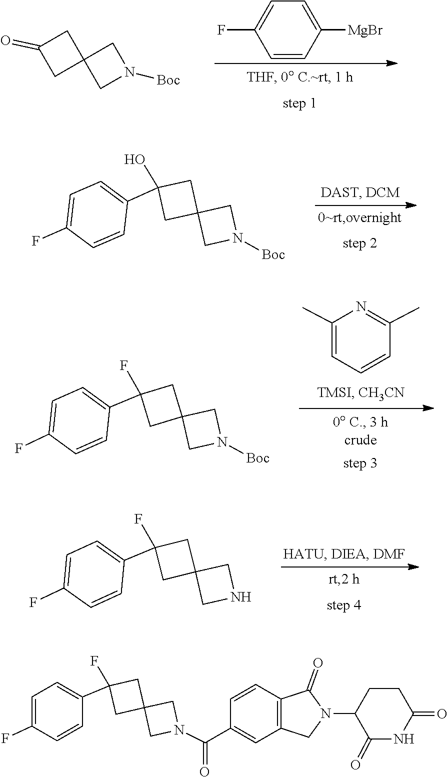

Step 1. Preparation of tert-butyl 6-(4-fluorophenyl)-6-hydroxy-2-azaspiro[3.3]heptane-2-carboxylate. To a stirred mixture of tert-butyl 6-oxo-2-azaspiro[3.3]heptane-2-carboxylate (2 g, 9.467 mmol, 1 equiv) in THF (40 mL) was added bromo(4-fluorophenyl)magnesium (14.2 mL, 14.201 mmol, 1.5 equiv) dropwise at 0° C. under nitrogen atmosphere. The resulting mixture was stirred for an additional 1 h at room temperature. The reaction was quenched by the addition of sat. NH₄Cl (aq.) (15 mL) at 0° C. The resulting mixture was diluted with water (100 mL) and extracted with EtOAc (3×100 mL). The combined organic layers were washed with brine (3×200 mL), dried over anhydrous Na₂SO₄. After filtration, the filtrate was concentrated under reduced pressure. The residue was purified by reversed-phase flash chromatography to afford tert-butyl 6-(4-fluorophenyl)-6-hydroxy-2-azaspiro[3.3]heptane-2-carboxylate (1.7 g, 58%) as an off-white solid. LC-MS (ES, m/z)=308.0 [M+1]⁺.

Step 2. Preparation of tert-butyl 6-fluoro-6-(4-fluorophenyl)-2-azaspiro[3.3]heptane-2-carboxylate. To a stirred mixture of tert-butyl 6-(4-fluorophenyl)-6-hydroxy-2-azaspiro[3.3]heptane-2-carboxylate (700 mg, 2.277 mmol, 1 equiv) in DCM (14 mL) was added DAST (5.69 mL, 5.693 mmol, 2.5 equiv) dropwise at 0° C. under nitrogen atmosphere. The resulting mixture was stirred for additional overnight at room temperature. The reaction was quenched by the addition of Water (5 mL) at 0° C. The resulting mixture was diluted with water (50 mL) and extracted with EtOAc (3×30 mL). The combined organic layers were washed with brine (3×50 mL), dried over anhydrous Na₂SO₄. After filtration, the filtrate was concentrated under reduced pressure to afford tert-butyl 6-fluoro-6-(4-fluorophenyl)-2-azaspiro[3.3]heptane-2-carboxylate (556 mg, 79%) as a light yellow solid. LC-MS (ES, m/z)=310.0 [M+1]⁺.

Step 3. Preparation of 6-fluoro-6-(4-fluorophenyl)-2-azaspiro[3.3]heptane. To a stirred mixture of tert-butyl 6-fluoro-6-(4-fluorophenyl)-2-azaspiro[3.3]heptane-2-carboxylate (250 mg, 0.808 mmol, 1 equiv) in MeCN were added 2,6-lutidine (346.4 mg, 3.232 mmol, 4 equiv) dropwise at room temperature under air atmosphere. To the above mixture was added TMSI (323.4 mg, 1.616 mmol, 2 equiv) dropwise at 0° C. The resulting mixture was stirred for an additional 3 h at 0° C. The resulting mixture was diluted with water (50 mL) and extracted with CHCl₃ (3×30 mL). The combined organic layers were washed with brine (3×50 mL), dried over anhydrous Na₂SO₄. After filtration, the filtrate was concentrated under reduced pressure to afford 6-fluoro-6-(4-fluorophenyl)-2-azaspiro[3.3]heptane (150 mg, crude) as a light yellow solid. LC-MS (ES, m/z)=210.25 [M+1]⁺.

Step 4. Preparation of 3-(5-(6-fluoro-6-(4-fluorophenyl)-2-azaspiro[3.3]heptane-2-carbonyl)-1-oxoisoindolin-2-yl)piperidine-2,6-dione. To a stirred mixture of 6-fluoro-6-(4-fluorophenyl)-2-azaspiro[3.3]heptane (120 mg, 0.574 mmol, 1 equiv) and 2-(2,6-dioxopiperidin-3-yl)-1-oxo-3H-isoindole-5-carboxylic acid (165.3 mg, 0.574 mmol, 1 equiv) in DMF (2.4 mL) were added DIEA (0.3 mL, 1.722 mmol, 3 equiv) and HATU (327.1 mg, 0.861 mmol, 1.5 equiv) in portions at room temperature under air atmosphere. The resulting mixture was stirred for an additional 2 h at room temperature. The resulting mixture was diluted with water (50 mL) and extracted with EtOAc (3×20 mL). The combined organic layers were washed with brine (3×50 mL), dried over anhydrous Na₂SO₄. After filtration, the filtrate was concentrated under reduced pressure. The residue was purified by reversed-phase flash chromatography to give the crude product (50 mg), which was purified by Prep-HPLC to afford 3-(5-(6-fluoro-6-(4-fluorophenyl)-2-azaspiro[3.3]heptane-2-carbonyl)-1-oxoisoindolin-2-yl)piperidine-2,6-dione (13.3 mg, 4.45%) as an off-white solid. LC-MS (ES, m/z)=480.25 [M+1]⁺. ¹H NMR (400 MHz, DMSO-d₆) δ 11.01 (d, J=3.2 Hz, 1H), 7.89-7.69 (m, 3H), 7.50 (dq, J=13.5, 6.7, 6.3 Hz, 2H), 7.28-7.19 (m, 2H), 5.14 (dd, J=12.4, 6.3 Hz, 1H), 4.54-4.47 (m, 2H), 4.38 (d, J=6.0 Hz, 1H), 4.27 (d, J=16.1 Hz, 2H), 4.06 (s, 1H), 3.01-2.73 (m, 5H), 2.61 (d, J=17.6 Hz, 1H), 2.40 (dq, J=13.2, 7.6, 6.6 Hz, 1H), 2.02 (dd, J=11.7, 5.9 Hz, 1H).

### Example 169

### Synthesis of 3-(5-(6-(4-fluorophenyl)-2-azaspiro[3.3]hept-5-ene-2-carbonyl)-1-oxoisoindolin-2-yl)piperidine-2,6-dione

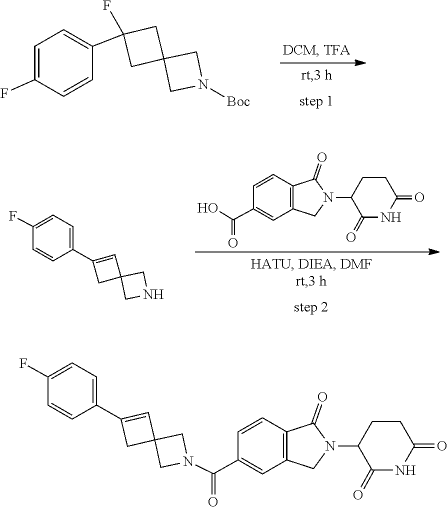 [SMILES: CC(C)(C)OC(=O)N1CC2(C1)CC(F)(c1ccc(F)cc1)C2 ; Fc1ccc(C2=CC3(CNC3)C2)cc1 ; O=C1CCC(N2Cc3cc(C(=O)O)ccc3C2=O)C(=O)N1 ; O=C1CCC(N2Cc3cc(C(=O)N4CC5(C=C(c6ccc(F)cc6)C5)C4)ccc3C2=O)C(=O)N1]

Step 1. Preparation of 6-(4-fluorophenyl)-2-azaspiro[3.3]hept-5-ene. To a stirred mixture of tert-butyl 6-fluoro-6-(4-fluorophenyl)-2-azaspiro[3.3]heptane-2-carboxylate (500 mg, 1.616 mmol, 1 equiv) in DCM (10 mL) was added TFA (2 mL, 26.926 mmol, 16.66 equiv) dropwise at 0° C. under air atmosphere. The resulting mixture was stirred for an additional 3 h at room temperature. The resulting mixture was concentrated under vacuum. The residue was purified by reversed-phase flash chromatography to afford 6-(4-fluorophenyl)-2-azaspiro[3.3]hept-5-ene (250 mg, crude) as a light brown oil. LC-MS (ES, m/z)=190.0 [M+1]⁺.

Step 2. Preparation of 3-(5-(6-(4-fluorophenyl)-2-azaspiro[3.3]hept-5-ene-2-carbonyl)-1-oxoisoindolin-2-yl)piperidine-2,6-dione. To a stirred mixture of 6-(4-fluorophenyl)-2-azaspiro[3.3]hept-5-ene (250 mg, 1.321 mmol, 1 equiv) and 2-(2,6-dioxopiperidin-3-yl)-1-oxo-3H-isoindole-5-carboxylic acid (457 mg, 1.585 mmol, 1.2 equiv) in DMF (5 mL) were added DIEA (0.7 mL, 3.963 mmol, 3 equiv) and HATU (753.517 mg, 1.982 mmol, 1.5 equiv) in portions at room temperature under air atmosphere. The resulting mixture was stirred for an additional 3 h at room temperature. The resulting mixture was diluted with water (50 mL) and extracted with EtOAc (3×30 mL). The combined organic layers were washed with brine (3×50 mL), dried over anhydrous Na₂SO₄. After filtration, the filtrate was concentrated under reduced pressure. The residue was purified by reversed-phase flash chromatography to give the crude product (100 mg), which was purified by Prep-HPLC to afford 3-(5-(6-(4-fluorophenyl)-2-azaspiro[3.3]hept-5-ene-2-carbonyl)-1-oxoisoindolin-2-yl)piperidine-2,6-dione (27.4 mg, 4.31%) as an off-white solid. LC-MS (ES, m/z)=460.2 [M+1]⁺. ¹H NMR (400 MHz, DMSO-d₆) δ 11.01 (s, 1H), 7.89 (s, 1H), 7.79 (s, 2H), 7.45 (dd, J=8.4, 5.4 Hz, 2H), 7.21 (dd, J=10.2, 7.3 Hz, 2H), 6.51 (d, J=2.6 Hz, 1H), 5.14 (dd, J=13.3, 5.0 Hz, 1H), 4.52 (dt, J=21.0, 6.1 Hz, 3H), 4.41-4.25 (m, 3H), 3.00-2.91 (m, 3H), 2.61 (d, J=17.8 Hz, 1H), 2.42 (dd, J=13.1, 4.5 Hz, 1H), 2.03 (dd, J=12.2, 6.4 Hz, 1H).

### Example 170

### Synthesis of 3-(5-(3-fluoro-3-(4-fluorophenyl)azetidine-1-carbonyl)-1-oxoisoindolin-2-yl)piperidine-2,6-dione

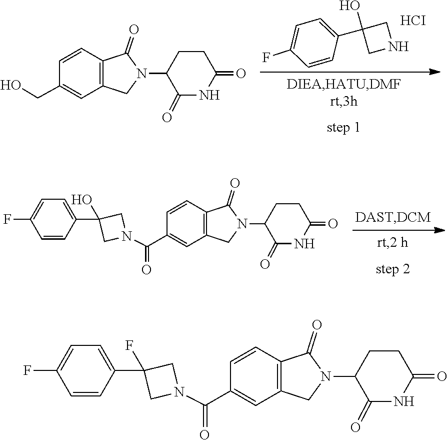

Step 1. Preparation of 3-(5-(3-(4-fluorophenyl)-3-hydroxyazetidine-1-carbonyl)-1-oxoisoindolin-2-yl)piperidine-2,6-dione. To a stirred mixture of 3-(4-fluorophenyl)azetidin-3-ol hydrochloride (200 mg, 0.982 mmol, 1 equiv) and 2-(2,6-dioxopiperidin-3-yl)-1-oxo-3H-isoindole-5-carboxylic acid (339.7 mg, 1.178 mmol, 1.2 equiv) in DMF (5 mL) were added DIEA (0.5 mL, 2.946 mmol, 3 equiv) and HATU (560.2 mg, 1.473 mmol, 1.5 equiv) in portions at room temperature under air atmosphere. The resulting mixture was stirred for an additional 3 h at room temperature. The resulting mixture was diluted with water (50 mL) and extracted with EtOAc (3×20 mL). The combined organic layers were washed with brine (3×50 mL), dried over anhydrous Na₂SO₄. After filtration, the filtrate was concentrated under reduced pressure. The residue was purified by reversed-phase flash chromatography to afford 3-(5-(3-(4-fluorophenyl)-3-hydroxyazetidine-1-carbonyl)-1-oxoisoindolin-2-yl)piperidine-2,6-dione (157 mg, 37%) as an off-white solid. LC-MS (ES, m/z)=438.3 [M+1]⁺.

Step 2. Preparation of 3-(5-(3-fluoro-3-(4-fluorophenyl)azetidine-1-carbonyl)-1-oxoisoindolin-2-yl)piperidine-2,6-dione. To a stirred mixture of 3-{5-[3-(4-fluorophenyl)-3-hydroxyazetidine-1-carbonyl]-1-oxo-3H-isoindol-2-yl}piperidine-2,6-dione (150 mg, 0.343 mmol, 1 equiv) in DCM (3 mL) was added DAST (138.2 mg, 0.858 mmol, 2.5 equiv) dropwise at 0° C. under nitrogen atmosphere. The resulting mixture was stirred for an additional 2 h at room temperature. The reaction was quenched by the addition of Water (5 mL) at 0° C. The resulting mixture was diluted with water (30 mL) and extracted with EtOAc (3×20 mL). The combined organic layers were washed with brine (3×50 mL), dried over anhydrous Na₂SO₄. After filtration, the filtrate was concentrated under reduced pressure. The residue was purified by reversed-phase flash chromatography to give the crude product (120 mg), which was purified by Prep-HPLC to afford 3-(5-(3-fluoro-3-(4-fluorophenyl)azetidine-1-carbonyl)-1-oxoisoindolin-2-yl)piperidine-2,6-dione (66 mg, 43%) as an off-white solid. LC-MS (ES, m/z)=440.2 [M+1]⁺. ¹H NMR (400 MHz, DMSO-d₆) δ 11.01 (s, 1H), 7.96 (s, 1H), 7.83 (dt, J=9.0, 7.5 Hz, 2H), 7.67-7.60 (m, 2H), 7.31 (t, J=8.7 Hz, 2H), 5.14 (dd, J=13.3, 4.9 Hz, 1H), 4.83 (dd, J=31.0, 18.4 Hz, 2H), 4.64-4.48 (m, 3H), 4.39 (d, J=17.5 Hz, 1H), 2.99-2.86 (m, 1H), 2.61 (d, J=17.0 Hz, 1H), 2.45-2.37 (m, 1H), 2.08-1.98 (m, 1H).

### Example 171

### Synthesis of 3-(6-(6-chloro-1,2,3,4-tetrahydroisoquinoline-2-carbonyl)benzo[d]oxazol-2-yl)piperidine-2,6-dione

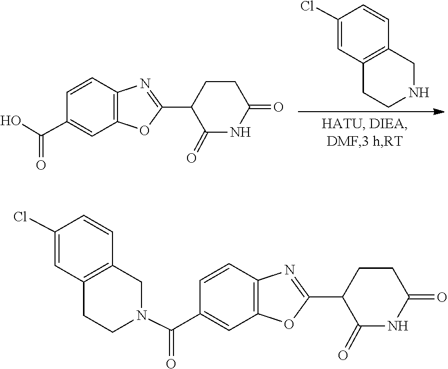 [SMILES: O=C1CCC(c2nc3ccc(C(=O)O)cc3o2)C(=O)N1 ; Clc1ccc2c(c1)CCNC2 ; O=C1CCC(c2nc3ccc(C(=O)N4CCc5cc(Cl)ccc5C4)cc3o2)C(=O)N1]

Step 1. Preparation of 3-(6-(6-chloro-1,2,3,4-tetrahydroisoquinoline-2-carbonyl)benzo[d]oxazol-2-yl)piperidine-2,6-dione. A solution of 2-(2,6-dioxopiperidin-3-yl)-1,3-benzoxazole-6-carboxylic acid (60 mg, 0.219 mmol, 1 equiv), DIEA (84.8 mg, 0.657 mmol, 3 equiv) and 6-chloro-1,2,3,4-tetrahydroisoquinoline (55.0 mg, 0.329 mmol, 1.5 equiv) in DMF (1 mL) was stirred for 2 min at room temperature. To the above mixture was added HATU (124.8 mg, 0.329 mmol, 1.5 equiv) in portions at room temperature. The resulting mixture was stirred for an additional 3 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(6-(6-chloro-1,2,3,4-tetrahydroisoquinoline-2-carbonyl)benzo[d]oxazol-2-yl)piperidine-2,6-dione (9 mg, 10%) as an off-white solid. LC-MS (ES, m/z)=424.1 [M+1]. ¹H NMR (400 MHz, DMSO-d₆) δ 11.16 (s, 1H), 7.93-7.79 (m, 2H), 7.47 (d, J=8.1 Hz, 1H), 7.29 (s, 3H), 4.85-4.49 (m, 3H), 3.70 (d, J=112.6 Hz, 3H), 2.90 (d, J=16.5 Hz, 3H), 2.77-2.60 (m, 2H), 2.43 (td, J=11.6, 4.3 Hz, 1H), 2.34 (dq, J=8.6, 4.9 Hz, 1H).

### Example 172

### Synthesis of 3-(5-(5-chloro-4-fluoroisoindoline-2-carbonyl)-1-oxoisoindolin-2-yl)piperidine-2,6-dione

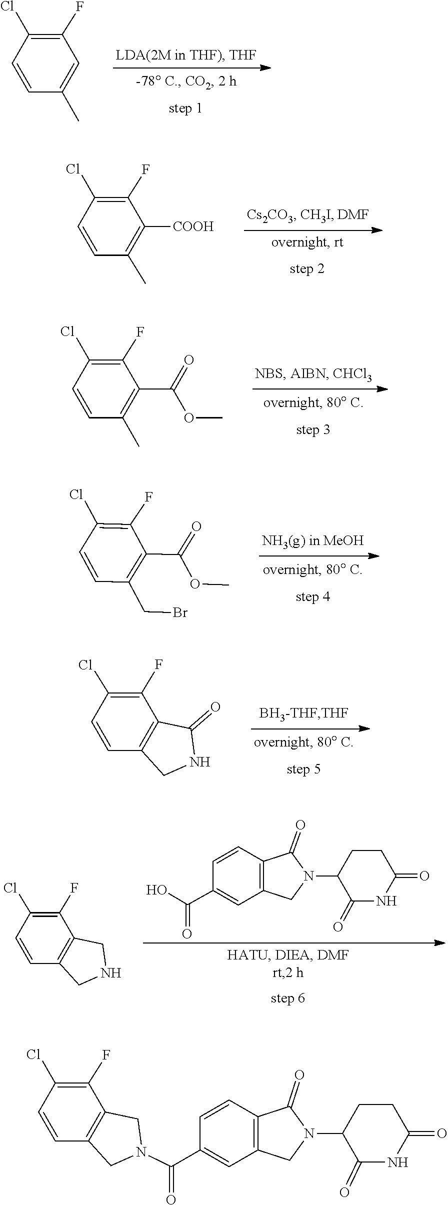

Step 1. Preparation of 3-chloro-2-fluoro-6-methylbenzoic acid. A solution of 1-chloro-2-fluoro-4-methylbenzene (10 g, 69.171 mmol, 1 equiv) in THF (150 mL) was treated with LDA (2M in THF) (41.0 mL, 1.2 equiv) for 1 h at −78° C. under nitrogen atmosphere followed by the addition of carbon dioxide for 30 min at −78° C. The resulting mixture was stirred for 2 h at 78° C. under nitrogen atmosphere. The reaction was quenched with sat. NH₄Cl (aq.) at 0° C. The residue was acidified with HCl (aq.). The resulting mixture was extracted with EtOAc (3×200 mL). The combined organic layers were washed with brine (3×100 mL), dried over anhydrous Na₂SO₄. After filtration, the filtrate was concentrated under reduced pressure. The residue was purified by reversed-phase flash chromatography to afford 3-chloro-2-fluoro-6-methylbenzoic acid (5.5 g, 42%) as a yellow oil. LC-MS (ES, m/z)=187.05 [M+1].

Step 2. Preparation of methyl 3-chloro-2-fluoro-6-methylbenzoate. To a stirred mixture of 3-chloro-2-fluoro-6-methylbenzoic acid (6 g, 31.817 mmol, 1 equiv) and Cs₂CO₃ (20.73 g, 63.634 mmol, 2 equiv) in DMF (120 mL) was added CH₃I (9.03 g, 63.634 mmol, 2 equiv) dropwise at 0° C. under nitrogen atmosphere. The resulting mixture was stirred overnight at room temperature under nitrogen atmosphere. The resulting mixture was diluted with water and extracted with EtOAc (3×200 mL). The combined organic layers were washed with brine (3×500 mL), dried over anhydrous Na₂SO₄. After filtration, the filtrate was concentrated under reduced pressure. The residue was purified by reversed-phase flash chromatography to afford methyl 3-chloro-2-fluoro-6-methylbenzoate (2.5 g, 39%) as a yellow oil. LC-MS (ES, m/z)=no mass detected.

Step 3. Preparation of methyl 6-(bromomethyl)-3-chloro-2-fluorobenzoate. A mixture of methyl 3-chloro-2-fluoro-6-methylbenzoate (2.5 g, 12.339 mmol, 1 equiv), NBS (3.29 g, 18.509 mmol, 1.5 equiv) and AIBN (0.20 g, 1.234 mmol, 0.1 equiv) in CHCl₃ (18 mL) was stirred overnight at 80° C. under nitrogen atmosphere. The resulting mixture was diluted with water and extracted with CH₂Cl₂ (3×100 mL). The combined organic layers were washed with brine (3×200 mL), dried over anhydrous Na₂SO₄. After filtration, the filtrate was concentrated under reduced pressure to afford methyl 6-(bromomethyl)-3-chloro-2-fluorobenzoate (3.3 g, 95%) as a brown oil. LC-MS (ES, m/z)=no mass detected.

Step 4. Preparation of 6-chloro-7-fluoroisoindolin-1-one. A mixture of methyl 6-(bromomethyl)-3-chloro-2-fluorobenzoate (3.3 g, 11.722 mmol, 1 equiv) in NH₃ (g) in MeOH (66 mL) was stirred overnight at 80° C. under nitrogen atmosphere. The resulting mixture was concentrated under reduced pressure. The residue was purified by silica gel column chromatography, eluted with CH₂Cl₂/MeOH (12:1) to afford 6-chloro-7-fluoroisoindolin-1-one (1.45 g, 67%) as a yellow solid. LC-MS (ES, m/z)=186.05 [M+1]⁺.

Step 5. Preparation of 5-chloro-4-fluoroisoindoline. To a stirred mixture of 6-chloro-7-fluoroisoindolin-1-one (1.45 g, 7.813 mmol, 1 equiv) in THF (9 ml) was added BH₃-THF (38 mL, 1.0M) dropwise at 0° C. under nitrogen atmosphere. The resulting mixture was stirred for overnight at 80° C. under nitrogen atmosphere. The reaction was quenched with Water at 0° C. The residue was acidified with 1M HCl (aq) and concentrated under reduced pressure. The residue was purified by reversed-phase flash chromatography to afford 5-chloro-4-fluoroisoindoline (1.4 g, crude) as a pink solid. LC-MS (ES, m/z)=172.05 [M+1]⁺.

Step 6. Preparation of 3-(5-(5-chloro-4-fluoroisoindoline-2-carbonyl)-1-oxoisoindolin-2-yl)piperidine-2,6-dione. A mixture of 5-chloro-4-fluoroisoindoline (100 mg, 0.583 mmol, 1 equiv), 2-(2,6-dioxopiperidin-3-yl)-1-oxo-3H-isoindole-5-carboxylic acid (251.97 mg, 0.874 mmol, 1.5 equiv), HATU (332.37 mg, 0.874 mmol, 1.5 equiv) and DIEA (225.96 mg, 1.749 mmol, 3 equiv) in DMF (1.5 mL) was stirred for 2 h at room temperature under air atmosphere. The resulting mixture was diluted with water and extracted with EtOAc (3×15 mL). The combined organic layers were washed with brine (3×20 mL), dried over anhydrous Na₂SO₄. After filtration, the filtrate was concentrated under reduced pressure. The residue was purified by reversed-phase flash chromatography to afford 3-(5-(5-chloro-4-fluoroisoindoline-2-carbonyl)-1-oxoisoindolin-2-yl)piperidine-2,6-dione (5.1 mg, 1.92%) as an off-white solid. LC-MS (ES, m/z)=440.05 [M−1]⁻. ¹H NMR (400 MHz, DMSO-d₆) δ 11.02 (s, 1H), 7.93-7.80 (m, 1H), 7.72 (d, J=7.9 Hz, 1H), 7.54 (dt, J=14.5, 7.5 Hz, 1H), 7.31 (d, J=8.2 Hz, 1H), 7.15 (d, J=8.2 Hz, 1H), 5.16 (dd, J=13.4, 5.2 Hz, 1H), 5.01-4.76 (m, 3H), 4.54 (dd, J=17.6, 7.2 Hz, 1H), 4.41 (dd, J=17.5, 5.8 Hz, 1H), 3.02-2.82 (m, 2H), 2.64 (s, 1H), 2.43 (d, J=13.0 Hz, 1H), 2.04 (d, J=13.3 Hz, 1H), 1.24 (s, 1H).

### Example 173

### Synthesis of 2-(2-(2,6-dioxopiperidin-3-yl)benzo[d]oxazole-6-carbonyl)isoindoline-4-carbonitrile

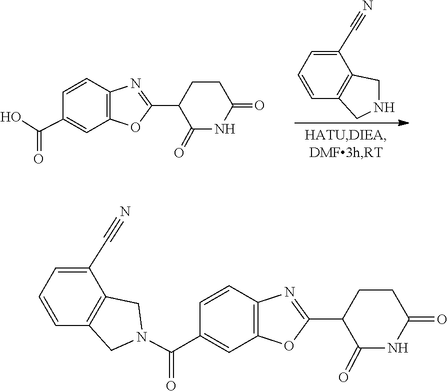 [SMILES: O=C1CCC(c2nc3ccc(C(=O)O)cc3o2)C(=O)N1 ; N#Cc1cccc2c1CNC2 ; N#Cc1cccc2c1CN(C(=O)c1ccc3nc(C4CCC(=O)NC4=O)oc3c1)C2]

Step 1. Preparation of 2-(2-(2,6-dioxopiperidin-3-yl)benzo[d]oxazole-6-carbonyl)isoindoline-4-carbonitrile. A solution of 2-(2,6-dioxopiperidin-3-yl)-1,3-benzoxazole-6-carboxylic acid (80 mg, 0.292 mmol, 1 equiv), DIEA (113.1 mg, 0.876 mmol, 3 equiv) and 2,3-dihydro-1H-isoindole-4-carbonitrile (84.1 mg, 0.584 mmol, 2 equiv) in DMF (1.5 mL) was stirred for 2 min at room temperature. To the above mixture was added HATU (166.3 mg, 0.438 mmol, 1.5 equiv) at room temperature. The resulting mixture was stirred for an additional 3 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to give the crude product (30 mg), which was purified by Prep-HPLC to afford 2-(2-(2,6-dioxopiperidin-3-yl)benzo[d]oxazole-6-carbonyl)isoindoline-4-carbonitrile (5 mg, 4.28%) as an off-white solid. LC-MS (ES, m/z)=401.15 [M+1]⁺. ¹H NMR (400 MHz, DMSO-d₆) δ 11.18 (s, 1H), 8.09 (d, J=1.4 Hz, 1H), 7.88 (dd, J=8.2, 4.2 Hz, 1H), 7.80 (t, J=7.9 Hz, 2H), 7.69-7.47 (m, 2H), 5.09-4.86 (m, 4H), 4.58 (dd, J=11.2, 5.1 Hz, 1H), 2.84-2.57 (m, 2H), 2.41 (d, J=23.6 Hz, 2H).

### Example 174

### Synthesis of 3-(6-(4-(trifluoromethyl)isoindoline-2-carbonyl)benzo[d]oxazol-2-yl)piperidine-2,6-dione

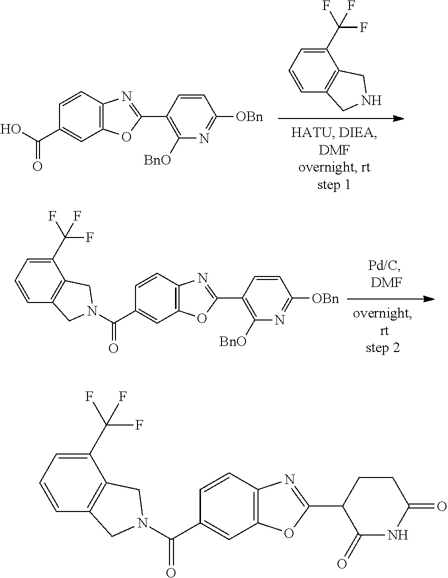 [SMILES: O=C(O)c1ccc2nc(-c3ccc(OCc4ccccc4)nc3OCc3ccccc3)oc2c1 ; FC(F)(F)c1cccc2c1CNC2 ; O=C(c1ccc2nc(-c3ccc(OCc4ccccc4)nc3OCc3ccccc3)oc2c1)N1Cc2cccc(C(F)(F)F)c2C1 ; O=C1CCC(c2nc3ccc(C(=O)N4Cc5cccc(C(F)(F)F)c5C4)cc3o2)C(=O)N1]

Step 1. Preparation of (2-(2,6-bis(benzyloxy)pyridin-3-yl)benzo[d]oxazol-6-yl)(4-(trifluoromethyl)isoindolin-2-yl)methanone. To a stirred mixture of 4-(trifluoromethyl)-2,3-dihydro-1H-isoindole (40 mg, 0.214 mmol, 1 equiv) and 2-[2,6-bis(benzyloxy)pyridin-3-yl]-1,3-benzoxazole-6-carboxylic acid (96.7 mg, 0.214 mmol, 1 equiv) in ACN (2.00 mL) was added TCFH (89.9 mg, 0.321 mmol, 1.5 equiv), NMI (70.1 mg, 0.856 mmol, 4 equiv) at room temperature. The resulting mixture was stirred overnight at room temperature. The resulting mixture was diluted with water (20 mL) and extracted with EtOAc (3×10 mL). The combined organic layers were washed with brine (2×10 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(4-(trifluoromethyl)isoindolin-2-yl)methanone (100 mg, 75%) as a white solid. LC-MS (ES, m/z)=622.5 [M+1]⁺.

Step 2. Preparation of 3-(6-(4-(trifluoromethyl)isoindoline-2-carbonyl)benzo[d]oxazol-2-yl)piperidine-2,6-dione. To a stirred mixture of (2-(2,6-bis(benzyloxy)pyridin-3-yl)benzo[d]oxazol-6-yl)(4-(trifluoromethyl)isoindolin-2-yl)methanone (100 mg, 0.161 mmol, 1 equiv) in DMF (1 mL) was added Pd/C (100 mg, Palladium 10% on Carbon (wetted with ca. 55% Water)) at room temperature. The resulting mixture was stirred overnight at room temperature under hydrogen atmosphere. The resulting mixture was filtered, the filter cake was washed with EtOAc (3×10 mL). The filtrate was concentrated under reduced pressure. The crude product was purified by Prep-HPLC to afford 3-(6-(4-(trifluoromethyl)isoindoline-2-carbonyl)benzo[d]oxazol-2-yl)piperidine-2,6-dione (36 mg, 50%) as a light blue solid. LC-MS (ES, m/z)=442.1 [M+1]⁺. ¹H NMR (400 MHz, DMSO-d₆) δ 11.17 (s, 1H), 8.08 (d, J=1.6 Hz, 1H), 7.86 (d, J=8.2 Hz, 1H), 7.71-7.50 (m, 4H), 5.04 (s, 1H), 4.94 (d, J=32.7 Hz, 3H), 4.58 (dd, J=11.5, 4.9 Hz, 1H), 2.69 (td, J=17.7, 17.2, 12.4 Hz, 2H), 2.47-2.40 (m, 1H), 2.36 (s, 1H).

### Example 175

### Synthesis of 3-(6-(4,6-difluoroindoline-1-carbonyl)benzo[d]oxazol-2-yl)piperidine-2,6-dione

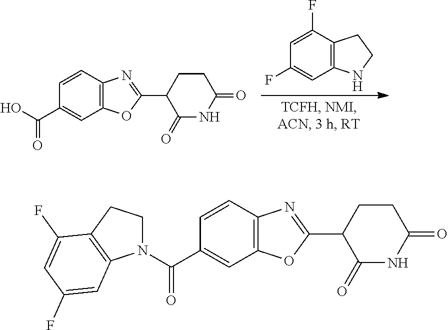

Step 1. Preparation of 3-(6-(4,6-difluoroindoline-1-carbonyl)benzo[d]oxazol-2-yl)piperidine-2,6-dione. A solution of 4,6-difluoro-2,3-dihydro-1H-indole (100 mg, 0.645 mmol, 1 equiv), NMI (211.6 mg, 2.580 mmol, 4 equiv) and 2-(2,6-dioxopiperidin-3-yl)-1,3-benzoxazole-6-carboxylic acid (265.1 mg, 0.968 mmol, 1.5 equiv) in ACN (2 mL) was stirred for 3 min at room temperature. To the above mixture was added TCFH (271.2 mg, 0.968 mmol, 1.5 equiv) in portions at room temperature. The resulting mixture was stirred for an additional 3 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography the crude product (30 mg), which was purified by Prep-HPLC to afford 3-(6-(4,6-difluoroindoline-1-carbonyl)benzo[d]oxazol-2-yl)piperidine-2,6-dione (6.8 mg, 2.56%) as an off-white solid. LC-MS (ES, m/z)=412.05 [M+1]⁺. ¹H NMR (400 MHz, DMSO-d₆) δ 11.16 (s, 1H), 8.07 (s, 1H), 7.87 (d, J=8.2 Hz, 1H), 7.64 (d, J=8.3 Hz, 2H), 6.95 (t, J=9.5 Hz, 1H), 4.58 (dd, J=11.4, 5.0 Hz, 1H), 4.14 (t, J=8.4 Hz, 2H), 3.08 (t, J=8.3 Hz, 3H), 2.82-2.60 (m, 3H), 2.42 (dd, J=11.5, 4.6 Hz, 1H), 2.39-2.30 (m, 1H).

### Example 176

### Synthesis of 3-(6-(4,6-difluoroisoindoline-2-carbonyl)benzo[d]oxazol-2-yl)piperidine-2,6-dione

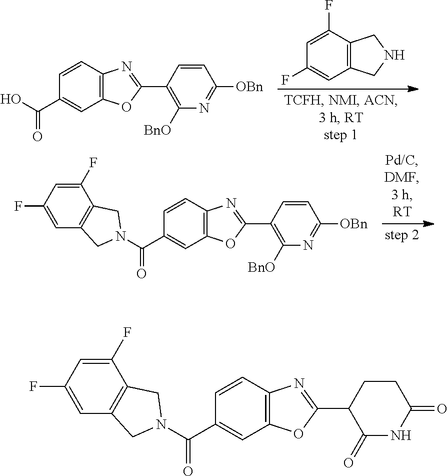 [SMILES: O=C(O)c1ccc2nc(-c3ccc(OCc4ccccc4)nc3OCc3ccccc3)oc2c1 ; Fc1cc(F)c2c(c1)CNC2 ; O=C(c1ccc2nc(-c3ccc(OCc4ccccc4)nc3OCc3ccccc3)oc2c1)N1Cc2cc(F)cc(F)c2C1 ; O=C1CCC(c2nc3ccc(C(=O)N4Cc5cc(F)cc(F)c5C4)cc3o2)C(=O)N1]

Step 1. Preparation of (2-(2,6-bis(benzyloxy)pyridin-3-yl)benzo[d]oxazol-6-yl)(4,6-difluoroisoindolin-2-yl)methanone. A solution of 4,6-difluoro-2,3-dihydro-1H-isoindole (102.8 mg, 0.663 mmol, 1.5 equiv), NMI (145.1 mg, 1.768 mmol, 4 equiv) and 2-[2,6-bis(benzyloxy)pyridin-3-yl]-1,3-benzoxazole-6-carboxylic acid (200 mg, 0.442 mmol, 1.00 equiv) in ACN (3 mL) was stirred for 2 min at room temperature. To the above mixture was added TCFH (186.0 mg, 0.663 mmol, 1.5 equiv) in portions at room temperature. The resulting mixture was stirred for an additional 3 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(4,6-difluoroisoindolin-2-yl)methanone (180 mg, 69%) as an off-white solid. LC-MS (ES, m/z)=590.3 [M+1]⁺.

Step 2. Preparation of 3-(6-(4,6-difluoroisoindoline-2-carbonyl)benzo[d]oxazol-2-yl)piperidine-2,6-dione. A solution of (2-(2,6-bis(benzyloxy)pyridin-3-yl)benzo[d]oxazol-6-yl)(4,6-difluoroisoindolin-2-yl)methanone (200 mg, 0.339 mmol, 1 equiv) and Pd/C (360.9 mg, 10% on carbon (wetted with ca.55% water)) in DMF (2 mL) was stirred for 3 h at room temperature under hydrogen atmosphere. The resulting mixture was filtered, the filter cake was washed with THF (3×10 mL). The filtrate was concentrated under reduced pressure. The residue was purified by reversed-phase flash chromatography with the following conditions: column, C18 silica gel; mobile phase, MeCN in Water (0.1% FA), 0% to 80% gradient in 30 min; detector, UV 254 nm. The crude product obtained (90 mg) was purified by Prep-HPLC to afford 3-(6-(4,6-difluoroisoindoline-2-carbonyl)benzo[d]oxazol-2-yl)piperidine-2,6-dione (38 mg, 27%) as an off-white solid. LC-MS (ES, m/z)=412.15 [M+1]⁺. ¹H NMR (400 MHz, DMSO-d₆) δ 11.16 (s, 1H), 8.07 (d, J=6.7 Hz, 1H), 7.85 (d, J=8.2 Hz, 1H), 7.67-7.58 (m, 1H), 7.25-6.99 (m, 2H), 4.92 (d, J=13.1 Hz, 2H), 4.85 (s, 2H), 4.57 (dd, J=11.4, 5.0 Hz, 1H), 2.80-2.59 (m, 2H), 2.49-2.29 (m, 2H).

### Example 177

### Synthesis of 3-(6-(4,7-difluoroisoindoline-2-carbonyl)benzo[d]oxazol-2-yl)piperidine-2,6-dione

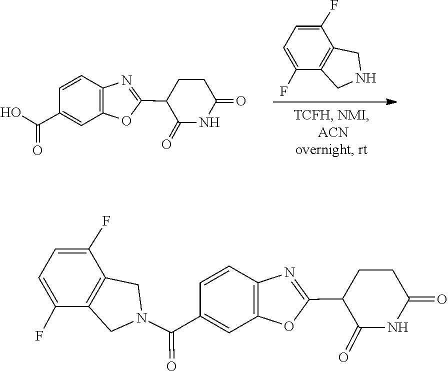 [SMILES: O=C1CCC(c2nc3ccc(C(=O)O)cc3o2)C(=O)N1 ; Fc1ccc(F)c2c1CNC2 ; O=C1CCC(c2nc3ccc(C(=O)N4Cc5c(F)ccc(F)c5C4)cc3o2)C(=O)N1]

Step 1. Preparation of 3-(6-(4,7-difluoroisoindoline-2-carbonyl)benzo[d]oxazol-2-yl)piperidine-2,6-dione. To a stirred mixture of 4,7-difluoro-2,3-dihydro-1H-isoindole (252 mg, 1.624 mmol, 5 equiv) and 2-(2,6-dioxopiperidin-3-yl)-1,3-benzoxazole-6-carboxylic acid (89.0 mg, 0.325 mmol, 1 equiv) in ACN (2 mL) were added TCFH (136.7 mg, 0.487 mmol, 1.5 equiv) and NMI (106.6 mg, 1.299 mmol, 4 equiv) at room temperature. The resulting mixture was stirred overnight at room temperature. The resulting mixture was diluted with water (20 mL) and extracted with EtOAc (3×10 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 40% to 50% gradient in 10 min; detector, UV 254 nm. This resulted in 3-(6-(4,7-difluoroisoindoline-2-carbonyl)benzo[d]oxazol-2-yl)piperidine-2,6-dione (18 mg, 13%) as a white solid. LC-MS (ES, m/z)=412.10 [M+1]⁺. ¹H NMR (400 MHz, DMSO-d₆) δ 11.16 (s, 1H), 8.07 (d, J=1.4 Hz, 1H), 7.86 (d, J=8.2 Hz, 1H), 7.65 (dd, J=8.2, 1.5 Hz, 1H), 7.22 (td, J=7.2, 3.9 Hz, 2H), 4.96 (d, J=19.4 Hz, 4H), 4.57 (dd, J=11.3, 5.0 Hz, 1H), 3.51 (s, 1H), 2.78-2.64 (m, 2H), 2.45-2.29 (m, 2H).

### Example 178

### Synthesis of 3-(6-(4-chloro-6-fluoroisoindoline-2-carbonyl)benzo[d]oxazol-2-yl)piperidine-2,6-dione

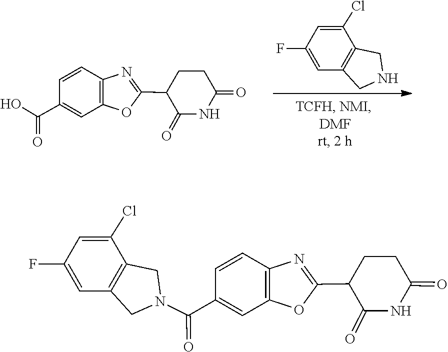 [SMILES: O=C1CCC(c2nc3ccc(C(=O)O)cc3o2)C(=O)N1 ; Fc1cc(Cl)c2c(c1)CNC2 ; O=C1CCC(c2nc3ccc(C(=O)N4Cc5cc(F)cc(Cl)c5C4)cc3o2)C(=O)N1]

Step 1. Preparation of 3-(6-(4-chloro-6-fluoroisoindoline-2-carbonyl)benzo[d]oxazol-2-yl)piperidine-2,6-dione. To a stirred mixture of 2-(2,4-dioxo-1,3-diazinan-1-yl)-1,3-benzoxazole-6-carboxylic acid (50 mg, 0.182 mmol, 1 equiv) and 4-chloro-6-fluoro-2,3-dihydro-1H-isoindole (34.3 mg, 0.200 mmol, 1.10 equiv) in DMF (1 mL) was added NMI (59.7 mg, 0.727 mmol, 4.00 equiv) and TCFH (76.5 mg, 0.273 mmol, 1.50 equiv) at room temperature under air atmosphere. The resulting mixture was stirred for 2 h at room temperature under the air atmosphere. The resulting mixture was diluted with water (20 mL) and the resulting mixture was extracted with EtOAc (3×10 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(6-(4-chloro-6-fluoroisoindoline-2-carbonyl)benzo[d]oxazol-2-yl)piperidine-2,6-dione (18 mg, 24%) as an off-white solid. LC-MS (ES, m/z)=428.05 [M+1]⁺. ¹H NMR (400 MHz, DMSO-d₆) δ 11.17 (s, 1H), 8.07 (s, 1H), 7.86 (d, J=8.0 Hz, 1H), 7.64 (t, J=8.2 Hz, 1H), 7.37 (dt, J=17.7, 8.9 Hz, 2H), 7.17 (d, J=8.0 Hz, 1H), 4.88 (dd, J=45.0, 30.4 Hz, 4H), 4.64-4.47 (m, 1H), 2.69 (q, J=17.9, 15.4 Hz, 2H), 2.36 (s, 2H).

### Example 179

### Synthesis of 3-(6-(8-chloro-1,2,3,4-tetrahydroisoquinoline-2-carbonyl)benzo[d]oxazol-2-yl)piperidine-2,6-dione

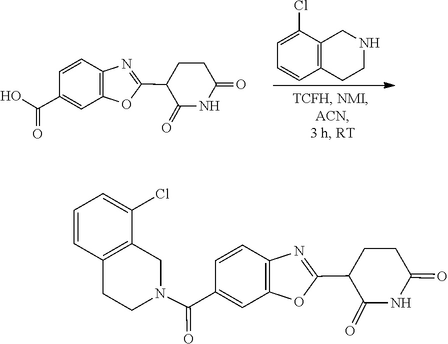 [SMILES: O=C1CCC(c2nc3ccc(C(=O)O)cc3o2)C(=O)N1 ; Clc1cccc2c1CNCC2 ; O=C1CCC(c2nc3ccc(C(=O)N4CCc5cccc(Cl)c5C4)cc3o2)C(=O)N1]

Step 1. Preparation of 3-(6-(8-chloro-1,2,3,4-tetrahydroisoquinoline-2-carbonyl)benzo[d]oxazol-2-yl)piperidine-2,6-dione. A solution of 8-chloro-1,2,3,4-tetrahydroisoquinoline (91.70 mg, 0.547 mmol, 1.5 equiv), NMI (119.7 mg, 1.460 mmol, 4 equiv) and 2-(2,6-dioxopiperidin-3-yl)-1,3-benzoxazole-6-carboxylic acid (100 mg, 0.365 mmol, 1.00 equiv) in ACN (3 mL) was stirred for 2 min at room temperature. To the above mixture was added TCFH (153.4 mg, 0.547 mmol, 1.5 equiv) in portions at room temperature. The resulting mixture was stirred for an additional 3 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The crude product (90 mg) was purified by Prep-HPLC to afford 3-(6-(8-chloro-1,2,3,4-tetrahydroisoquinoline-2-carbonyl)benzo[d]oxazol-2-yl)piperidine-2,6-dione (14 mg, 8.99%) as an off-white solid. LC-MS (ES, m/z)=424.15 [M+1]⁺. ¹H NMR (400 MHz, DMSO-d₆) δ 11.16 (s, 1H), 7.92 (d, J=1.4 Hz, 1H), 7.85 (d, J=8.1 Hz, 1H), 7.50 (dd, J=8.1, 1.5 Hz, 1H), 7.36 (s, 1H), 7.29-7.17 (m, 2H), 4.88-4.50 (m, 3H), 3.73 (d, J=120.2 Hz, 2H), 2.92 (d, J=7.4 Hz, 2H), 2.82-2.58 (m, 2H), 2.49-2.29 (m, 2H).

### Example 180

### Synthesis of 3-(6-(7-chloro-1,2,3,4-tetrahydroisoquinoline-2-carbonyl)benzo[d]oxazol-2-yl)piperidine-2,6-dione

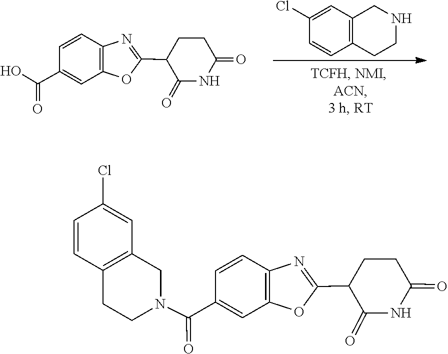 [SMILES: O=C1CCC(c2nc3ccc(C(=O)O)cc3o2)C(=O)N1 ; Clc1ccc2c(c1)CNCC2 ; O=C1CCC(c2nc3ccc(C(=O)N4CCc5ccc(Cl)cc5C4)cc3o2)C(=O)N1]

Step 1. Preparation of 3-(6-(7-chloro-1,2,3,4-tetrahydroisoquinoline-2-carbonyl)benzo[d]oxazol-2-yl)piperidine-2,6-dione. A solution of 7-chloro-1,2,3,4-tetrahydroisoquinoline (91.7 mg, 0.547 mmol, 1.5 equiv), NMI (119.7 mg, 1.460 mmol, 4 equiv) and 2-(2,6-dioxopiperidin-3-yl)-1,3-benzoxazole-6-carboxylic acid (100 mg, 0.365 mmol, 1.00 equiv) in ACN (3 mL) was stirred for 2 min at room temperature. To the above mixture was added TCFH (153.4 mg, 0.547 mmol, 1.5 equiv) in portions at room temperature. The resulting mixture was stirred for an additional 3 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The crude product (80 mg) was purified by Prep-HPLC to afford 3-(6-(7-chloro-1,2,3,4-tetrahydroisoquinoline-2-carbonyl)benzo[d]oxazol-2-yl)piperidine-2,6-dione (15 mg, 9.64%) as an off-white solid. LC-MS (ES, m/z)=424.15 [M+1]⁺. ¹H NMR (400 MHz, DMSO-d₆) δ 11.16 (s, 1H), 7.89 (s, 1H), 7.83 (d, J=8.2 Hz, 1H), 7.51-7.09 (m, 4H), 5.03-4.46 (m, 3H), 3.85 (s, 1H), 2.85 (s, 2H), 2.80-2.60 (m, 2H), 2.43 (td, J=12.0, 11.4, 4.7 Hz, 1H), 2.33 (dq, J=13.7, 5.0 Hz, 1H).

### Example 181

### Synthesis of 3-(6-(5-chloro-1,2,3,4-tetrahydroisoquinoline-2-carbonyl)benzo[d]oxazol-2-yl)piperidine-2,6-dione

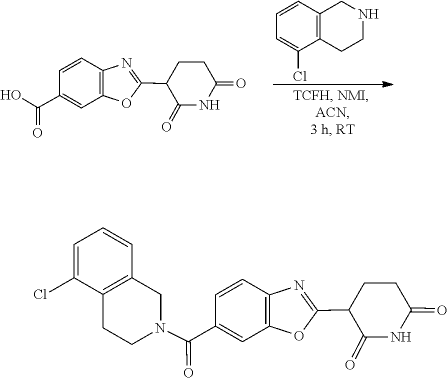 [SMILES: O=C1CCC(c2nc3ccc(C(=O)O)cc3o2)C(=O)N1 ; Clc1cccc2c1CCNC2 ; O=C1CCC(c2nc3ccc(C(=O)N4CCc5c(Cl)cccc5C4)cc3o2)C(=O)N1]

Step 1. Preparation of 3-(6-(5-chloro-1,2,3,4-tetrahydroisoquinoline-2-carbonyl)benzo[d]oxazol-2-yl)piperidine-2,6-dione. A solution of 5-chloro-1,2,3,4-tetrahydroisoquinoline (91.70 mg, 0.547 mmol, 1.5 equiv), NMI (119.7 mg, 1.460 mmol, 4 equiv) and 2-(2,6-dioxopiperidin-3-yl)-1,3-benzoxazole-6-carboxylic acid (100 mg, 0.365 mmol, 1.00 equiv) in ACN (3 mL) was stirred for 2 min at room temperature. To the above mixture was added TCFH (153.4 mg, 0.547 mmol, 1.5 equiv) in portions at room temperature. The resulting mixture was stirred for an additional 3 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The crude product (90 mg) was purified by Prep-HPLC to afford 3-(6-(5-chloro-1,2,3,4-tetrahydroisoquinoline-2-carbonyl)benzo[d]oxazol-2-yl)piperidine-2,6-dione (17 mg, 11%) as an off-white solid. LC-MS (ES, m/z)=424.15 [M+1]⁺. ¹H NMR (400 MHz, DMSO-d₆) δ 11.16 (s, 1H), 7.91 (s, 1H), 7.84 (d, J=8.2 Hz, 1H), 7.49 (d, J=8.2 Hz, 1H), 7.38-7.31 (m, 1H), 7.15 (d, J=80.6 Hz, 2H), 4.95-4.47 (m, 3H), 3.96-3.54 (m, 2H), 2.87 (t, J=6.1 Hz, 2H), 2.81-2.60 (m, 2H), 2.39 (dtd, J=34.9, 10.7, 8.6, 4.8 Hz, 2H), 2.08 (s, 1H).

### Example 182

### Synthesis of 3-(5-(7′-fluorospiro[cyclopropane-1,1′-isoindoline]-2′-carbonyl)-1-oxoisoindolin-2-yl)piperidine-2,6-dione

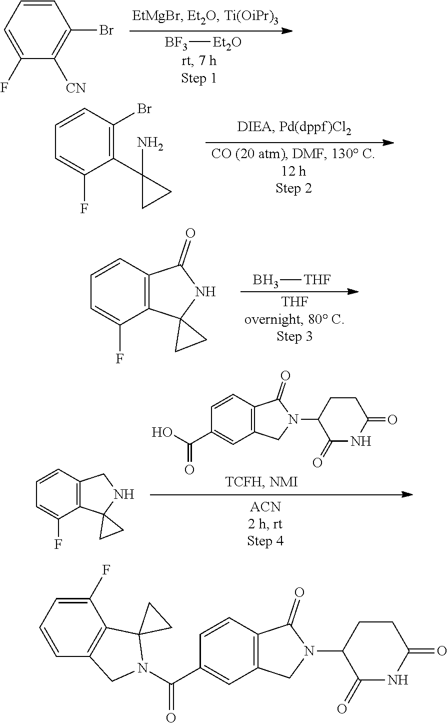

Step 1. Preparation of 1-(2-bromo-6-fluorophenyl)cyclopropan-1-amine. To a stirred mixture of 2-bromo-6-fluorobenzonitrile (5 g, 24.999 mmol, 1.00 equiv) in Et₂O (85 mL) was added tetrakis(propan-2-yloxy)titanium (8.22 mL, 27.749 mmol, 1.0 equiv) in portions at −78° C. under nitrogen atmosphere. To the above mixture was added EtMgBr (18.31 mL, 1.0 M in THF) dropwise at −78° C. The resulting mixture was stirred at −78° C. for an additional 1 h, the mixture was allowed to warm to RT and stirred for 3 h. To the above mixture was added boron trifluoride diethyl etherate (6.18 mL, 43.543 mmol, 1.8 equiv) dropwise at room temperature. The resulting mixture was stirred at room temperature for an additional 4 h. The reaction was quenched by the addition of Potassium sodium tartrate and sodium hydroxide (3:1) aqueous solution (120 mL) at room temperature. The resulting mixture was extracted with Et₂O (3×200 mL). The combined organic layers were washed with water (3×200 mL), dried over anhydrous Na₂SO₄. After filtration, the filtrate was concentrated under reduced pressure. The residue was purified by reversed-phase flash chromatography to afford 1-(2-bromo-6-fluorophenyl)cyclopropan-1-amine (1 g, 17%) as a yellow oil. LC-MS (ES, m/z)=230.15 [M+1]⁺.

Step 2. Preparation of 7′-fluorospiro[cyclopropane-1,1′-isoindolin]-3′-one. To a stirred mixture of 1-(2-bromo-6-fluorophenyl)cyclopropan-1-amine (1 g, 4.346 mmol, 1 equiv) and DIEA (2.25 g, 17.384 mmol, 4 equiv) in DMF (30.00 mL) was added Pd(dppf)Cl₂ (477.04 mg, 0.652 mmol, 0.15 equiv) at room temperature. The resulting mixture was stirred for an additional 12 h at 130° C. under CO (20 atm) atmosphere. The reaction was diluted with water (200 mL) and extracted with EtOAc (3×100 mL). The combined organic layers were washed with water (2×200 mL), dried over anhydrous Na₂SO₄. After filtration, the filtrate was concentrated under reduced pressure. The residue was purified by reversed-phase flash chromatography to afford 7′-fluorospiro[cyclopropane-1,1′-isoindolin]-3′-one (400 mg, 52%) as a brown solid. LC-MS (ES, m/z)=178.10 [M+1]⁺.

Step 3. Preparation of 7′-fluorospiro[cyclopropane-1,1′-isoindoline]. To a stirred solution of 7′-fluorospiro[cyclopropane-1,1′-isoindolin]-3′-one (400 mg, 2.258 mmol, 1 equiv) in tetrahydrofuran (40 mL) was added BH₃-THF (8.00 mL, 83.614 mmol, 37.03 equiv) under nitrogen atmosphere. The resulting mixture was stirred overnight at 80° C. under nitrogen atmosphere. The reaction was quenched by the addition of Water (100 mL) and extracted with EtOAc (3×50 mL). The combined organic layers were washed with water (2×100 mL), dried over anhydrous Na₂SO₄. After filtration, the filtrate was concentrated under reduced pressure. The residue was purified by reversed-phase flash chromatography to afford 7′-fluorospiro[cyclopropane-1,1′-isoindoline] (200 mg, 54%) as an off-white solid. LC-MS (ES, m/z)=164.0 [M+1]⁺.

Step 4. Preparation of 7′-fluorospiro[cyclopropane-1,1′-isoindoline]. To a stirred mixture of 2-(2,6-dioxopiperidin-3-yl)-1-oxo-3H-isoindole-5-carboxylic acid (80 mg, 0.278 mmol, 1.00 equiv) and 7′-fluorospiro[cyclopropane-1,1′-isoindoline] (90.58 mg, 0.556 mmol, 2 equiv) in ACN (1.5 mL) were added NMI (113.93 mg, 1.390 mmol, 5 equiv) and TCFH (155.74 mg, 0.556 mmol, 2 equiv) in portions at room temperature under nitrogen atmosphere. The resulting mixture was stirred for an additional 2 h at room temperature. The reaction was diluted with water (50 mL) and extracted with EtOAc (3×30 mL). The combined organic layers were washed with water (2×50 mL), dried over anhydrous Na₂SO₄. After filtration, the filtrate was concentrated under reduced pressure. The crude product (50 mg) was purified by Prep-HPLC to afford 3-[5-((7′-fluoro-3′H-spiro[cyclopropane-1,1′-isoindol]-2′-ylcarbonyl)-1-oxo-3H-isoindol-2-yl]piperidine-2,6-dione (17 mg, 16%) as an off-white solid. LC-MS (ES, m/z)=434.20 [M+1]⁺. ¹H NMR (400 MHz, DMSO-d₆) δ 11.01 (s, 1H), 7.83-7.77 (m, 1H), 7.74 (s, 1H), 7.61 (dd, J=7.7, 1.4 Hz, 1H), 7.27 (td, J=7.9, 4.9 Hz, 1H), 7.09 (t, J=9.3 Hz, 2H), 5.15 (dd, J=13.3, 5.1 Hz, 1H), 4.79 (s, 2H), 4.51 (d, J=17.5 Hz, 1H), 4.38 (d, J=17.4 Hz, 1H), 2.93 (ddd, J=17.8, 13.6, 5.4 Hz, 1H), 2.61 (d, J=17.2 Hz, 1H), 2.47-2.37 (m, 2H), 2.03 (dd, J=9.2, 3.9 Hz, 1H), 1.33 (q, J=4.6 Hz, 2H).

### Example 183

### Synthesis of 3-(6-fluoro-1-oxo-5-(6-(trifluoromethyl)-2-azaspiro[3.3]heptane-2-carbonyl)isoindolin-2-yl)piperidine-2,6-dione

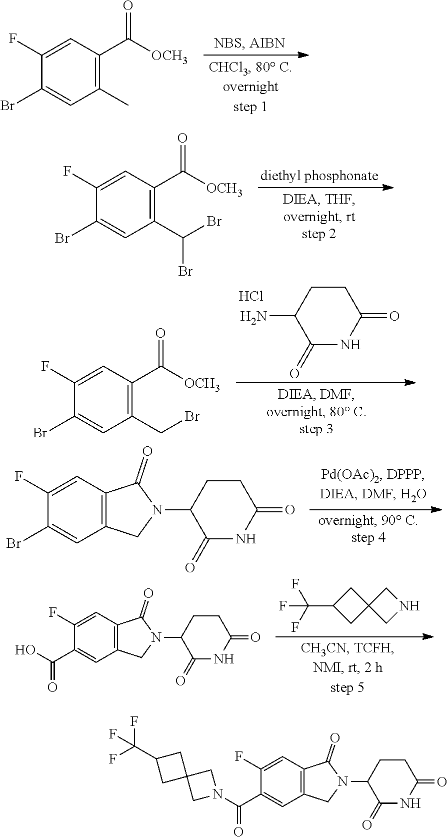

Step 1. Preparation of methyl 4-bromo-2-(dibromomethyl)-5-fluorobenzoate. A mixture of methyl 4-bromo-5-fluoro-2-methylbenzoate (2 g, 8.095 mmol, 1 equiv), NBS (5.76 g, 32.380 mmol, 4 equiv) and AIBN (130 mg, 0.810 mmol, 0.1 equiv) in CHCl₃ (30 mL) was stirred overnight at 80° C. under nitrogen atmosphere. The mixture was allowed to cool down to room temperature. The resulting mixture was diluted with water and extracted with DCM (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EA (1:1) to afford methyl 4-bromo-2-(dibromomethyl)-5-fluorobenzoate (3.2 g, 98%) as a yellow solid. LC-MS (ES, m/z)=no mass signal.

Step 2. Preparation of methyl 4-bromo-2-(bromomethyl)-5-fluorobenzoate. To a stirred mixture of methyl 4-bromo-2-(dibromomethyl)-5-fluorobenzoate (3 g, 7.410 mmol, 1 equiv) and DIEA (3.83 g, 29.640 mmol, 4 equiv) in THF (60 mL) was added diethyl phosphonate (4.09 g, 29.640 mmol, 4 equiv) in portions at 0° C. under nitrogen atmosphere. The resulting mixture was stirred overnight at room temperature under nitrogen atmosphere. The resulting mixture was diluted with water (100 mL). The resulting mixture was extracted with EtOAc (3×100 mL). The combined organic layers were washed with brine (3×300 mL), dried over anhydrous Na₂SO₄. After filtration, the filtrate was concentrated under reduced pressure. The residue was purified by silica gel column chromatography, eluted with PE/EA (1:1) to afford methyl 4-bromo-2-(bromomethyl)-5-fluorobenzoate (2.2 g, 91%) as a yellow oil. LC-MS (ES, m/z)=no mass signal.

Step 3. Preparation of 3-(5-bromo-6-fluoro-1-oxoisoindolin-2-yl)piperidine-2,6-dione. A mixture of methyl 4-bromo-2-(bromomethyl)-5-fluorobenzoate (2.2 g, 6.749 mmol, 1 equiv), 3-aminopiperidine-2,6-dione hydrochloride (3.33 g, 20.247 mmol, 3 equiv) and DIEA (5.23 g, 40.494 mmol, 6 equiv) in DMF (33 mL) was stirred for overnight at 80° C. under nitrogen atmosphere. The mixture was allowed to cool down to room temperature. The resulting mixture was diluted with water and extracted with EtOAc (3×50 mL). The combined organic layers were washed with brine (3×20 mL), dried over anhydrous Na₂SO₄. The resulting mixture was concentrated under reduced pressure. The residue was purified by reversed-phase flash chromatography to afford 3-(5-bromo-6-fluoro-1-oxoisoindolin-2-yl)piperidine-2,6-dione (2.15 g, 93%) as a dark blue solid. LC-MS (ES, m/z)=341.1 [M+1]⁺.

Step 4. Preparation of 2-(2,6-dioxopiperidin-3-yl)-6-fluoro-1-oxoisoindoline-5-carboxylic acid. A mixture of 3-(5-bromo-6-fluoro-1-oxoisoindolin-2-yl)piperidine-2,6-dione (1 g, 2.931 mmol, 1 equiv) and Pd(OAc)₂ (197.4 mg, 0.879 mmol, 0.3 equiv) and DPPP (362.7 mg, 0.879 mmol, 0.3 equiv) and DIEA (1.14 g, 8.793 mmol, 3 equiv) and H₂O (0.5 mL) in DMF (15 mL) was stirred overnight at 90° C. under nitrogen atmosphere. The resulting mixture was filtered, the filter cake was washed with DMF (2×5 mL). The resulting mixture was diluted with water and extracted with EtOAc (3×50 mL). The combined organic layers were washed with brine (3×30 mL), dried over anhydrous Na₂SO₄. After filtration, the filtrate was concentrated under reduced pressure. The residue was purified by reversed-phase flash chromatography to afford 2-(2,6-dioxopiperidin-3-yl)-6-fluoro-1-oxoisoindoline-5-carboxylic acid (600 mg, 67%) as a yellow oil. LC-MS (ES, m/z)=307.1 [M+1]⁺.

Step 5. Preparation of 3-(6-fluoro-1-oxo-5-(6-(trifluoromethyl)-2-azaspiro[3.3]heptane-2-carbonyl)isoindolin-2-yl)piperidine-2,6-dione. A mixture of 2-(2,6-dioxopiperidin-3-yl)-6-fluoro-1-oxoisoindoline-5-carboxylic acid (178.0 mg, 0.581 mmol, 1.2 equiv), 6-(trifluoromethyl)-2-azaspiro[3.3]heptane (80 mg, 0.484 mmol, 1.00 equiv) in ACN (3.0 mL) was added NMI (159.1 mg, 1.936 mmol, 4 equiv) and TCFH (271.8 mg, 0.968 mmol, 2 equiv) in portions at room temperature. The resulting mixture was stirred for 2 h at room temperature under nitrogen atmosphere. The resulting mixture was diluted with water and extracted with EtOAc (3×30 mL). The combined organic layers were washed with brine (3×20 mL), dried over anhydrous Na₂SO₄. After filtration, the filtrate was concentrated under reduced pressure. The residue was purified by reversed-phase flash chromatography to afford 3-(6-fluoro-1-oxo-5-(6-(trifluoromethyl)-2-azaspiro[3.3]heptane-2-carbonyl)isoindolin-2-yl)piperidine-2,6-dione (81 mg, 36%) as an off-white solid. LC-MS (ES, m/z)=454.1 [M+1]⁺. ¹H NMR (400 MHz, DMSO-d₆) δ 11.02 (s, 1H), 7.75 (t, J=5.9 Hz, 1H), 7.63 (dd, J=8.6, 4.6 Hz, 1H), 5.14 (dd, J=13.3, 5.1 Hz, 1H), 4.48 (d, J=17.4 Hz, 1H), 4.35 (d, J=17.4 Hz, 1H), 4.20-3.96 (m, 4H), 3.06 (dt, J=24.4, 8.8 Hz, 1H), 2.98-2.82 (m, 1H), 2.75-2.56 (m, 1H), 2.48-2.37 (m, 2H), 2.28 (ddd, J=20.6, 13.2, 7.1 Hz, 2H), 2.08-1.97 (m, 1H).

### Example 184

### Synthesis of 3-(1-oxo-5-(7-(trifluoromethoxy)-2-azaspiro[3.5]nonane-2-carbonyl)isoindolin-2-yl)piperidine-2,6-dione

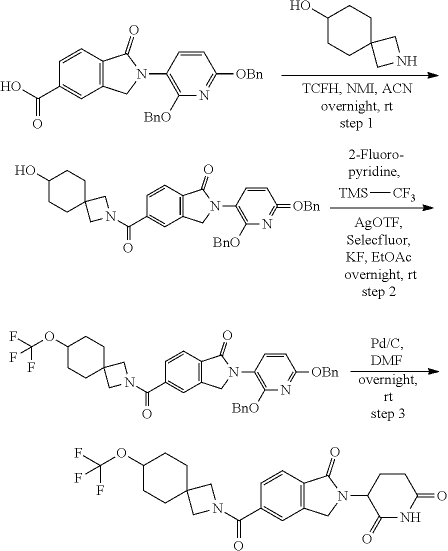

Step 1. Preparation of 2-(2,6-bis(benzyloxy)pyridin-3-yl)-5-(7-hydroxy-2-azaspiro[3.5]nonane-2-carbonyl)isoindolin-1-one. To a stirred mixture of 2-azaspiro[3.5]nonan-7-ol (300 mg, 2.124 mmol, 1 equiv) and 2-[2,6-bis(benzyloxy)pyridin-3-yl]-1-oxo-3H-isoindole-5-carboxylic acid (0.99 g, 2.124 mmol, 1 equiv) in ACN (10 mL) were added TCFH (894.1 mg, 3.186 mmol, 1.5 equiv) and NMI (697.7 mg, 8.496 mmol, 4 equiv) at room temperature. The resulting mixture was stirred overnight at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(7-hydroxy-2-azaspiro[3.5]nonane-2-carbonyl)isoindolin-1-one (1.1 g, 88%) as a white solid. LC-MS (ES, m/z)=590.0 [M+1]⁺.

Step 2. Preparation of 2-(2,6-bis(benzyloxy)pyridin-3-yl)-5-(7-(trifluoromethoxy)-2-azaspiro[3.5]nonane-2-carbonyl)isoindolin-1-one. To a stirred mixture of 2-(2,6-bis(benzyloxy)pyridin-3-yl)-5-(7-hydroxy-2-azaspiro[3.5]nonane-2-carbonyl)isoindolin-1-one (501 mg, 0.850 mmol, 1 equiv) and KF (197.4 mg, 3.400 mmol, 4 equiv) in EtOAc (10.0 mL) were added silver trifluoromethanesulfonate (873.1 mg, 3.400 mmol, 4 equiv), 2-Fluoropyridine (329.9 mg, 3.400 mmol, 4 equiv), 4-(chloromethyl)-1-fluoro-1,4-diazabicyclo[2.2.2]octane-1,4-diium (451.47 mg, 1.275 mmol, 1.5 equiv) and trimethyl(trifluoromethyl)silane (302.0 mg, 2.125 mmol, 2.5 equiv) at room temperature under nitrogen atmosphere. The resulting mixture was stirred overnight at room temperature under nitrogen atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(7-(trifluoromethoxy)-2-azaspiro[3.5]nonane-2-carbonyl)isoindolin-1-one (100 mg, 18%) as a white solid. LC-MS (ES, m/z)=658.0 [M+1]⁺.

Step 3. Preparation of 3-(1-oxo-5-(7-(trifluoromethoxy)-2-azaspiro[3.5]nonane-2-carbonyl)isoindolin-2-yl)piperidine-2,6-dione. To a stirred mixture of 2-(2,6-bis(benzyloxy)pyridin-3-yl)-5-(7-(trifluoromethoxy)-2-azaspiro[3.5]nonane-2-carbonyl)isoindolin-1-one (100 mg, 0.152 mmol, 1 equiv) in DMF (5 mL) was added Pd/C (50.1 mg, Palladium 10% on Carbon (wetted with ca. 55% Water)) at room temperature. The resulting mixture was stirred overnight at room temperature under hydrogen atmosphere. The resulting mixture was filtered, the filter cake was washed with DMF (3×2 mL). The filtrate was concentrated under reduced pressure. The residue was purified by reversed-phase flash chromatography to give the crude product (30 mg) was purified by Prep-HPLC to afford 3-(1-oxo-5-(7-(trifluoromethoxy)-2-azaspiro[3.5]nonane-2-carbonyl)isoindolin-2-yl)piperidine-2,6-dione (6.4 mg, 8.78%) as a white solid. LC-MS (ES, m/z)=480.1 [M+1]⁺. ¹H NMR (400 MHz, DMSO-d₆) δ 11.00 (s, 1H), 7.87 (s, 1H), 7.82-7.72 (m, 2H), 5.14 (dd, J=13.1, 5.0 Hz, 1H), 4.50 (dd, J=17.6, 2.8 Hz, 1H), 4.45-4.33 (m, 2H), 4.00 (d, J=13.4 Hz, 2H), 3.77 (d, J=13.2 Hz, 2H), 2.92 (ddd, J=18.1, 13.6, 5.4 Hz, 1H), 2.61 (d, J=17.1 Hz, 1H), 2.46-2.35 (m, 1H), 2.06-1.99 (m, 1H), 1.91-1.75 (m, 4H), 1.70-1.51 (m, 4H).

### Example 185

### Synthesis of 3-(5-(4-fluoro-7-(trifluoromethoxy)isoindoline-2-carbonyl)-1-oxoisoindolin-2-yl)piperidine-2,6-dione

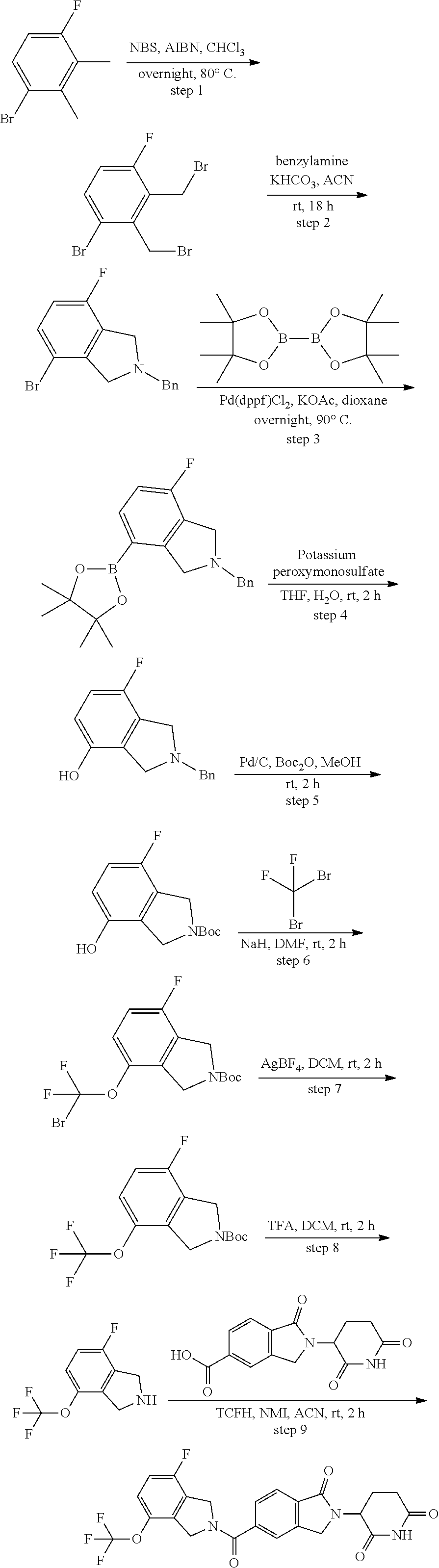

Step 1. Preparation of 1-bromo-2,3-bis(bromomethyl)-4-fluorobenzene. A mixture of 1-bromo-4-fluoro-2,3-dimethylbenzene (5 g, 24.624 mmol, 1 equiv), NBS (8.77 g, 49.248 mmol, 2 equiv) and AIBN (0.40 g, 2.462 mmol, 0.1 equiv) in CHCl₃ (80 mL) was stirred overnight at 80° C. under nitrogen atmosphere. The resulting mixture was diluted with water and extracted with DCM (3×200 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (12:1) to afford 1-bromo-2,3-bis(bromomethyl)-4-fluorobenzene (6.7 g, 75%) as a colorless oil. LC-MS (ES, m/z)=381 [M+Na]⁺.

Step 2. Preparation of 2-benzyl-4-bromo-7-fluoroisoindoline. A mixture of 1-bromo-2,3-bis(bromomethyl)-4-fluorobenzene (6.7 g, 18.567 mmol, 1 equiv) and phenylmethanamine (1.99 g, 18.567 mmol, 1 equiv) and KHCO₃ (4.65 g, 46.418 mmol, 2.5 equiv) in acetonitrile (700 mL) was stirred for 8 h at 80° C. under nitrogen atmosphere. The mixture was allowed to cool down to room temperature. The resulting mixture was stirred for 18 h at room temperature under nitrogen atmosphere. The resulting mixture was concentrated under reduced pressure. The resulting mixture was diluted with water (500 mL). The resulting mixture was extracted with EtOAc (3×200 mL). The combined organic layers were washed with brine (3×300 mL), dried over anhydrous Na₂SO₄. After filtration, the filtrate was concentrated under reduced pressure. The residue was purified by silica gel column chromatography, eluted with PE/EA (1:1) to afford 2-benzyl-4-bromo-7-fluoroisoindoline (1.7 g, 30%) as a brown oil. LC-MS (ES, m/z)=306.05 [M+1]⁺.

Step 3. Preparation of 2-benzyl-4-fluoro-7-(4,4,5,5-tetramethyl-1,3,2-dioxaborolan-2-yl)isoindoline. A mixture of 2-benzyl-4-bromo-7-fluoroisoindoline (1.1 g, 3.593 mmol, 1 equiv), 4,4,5,5-tetramethyl-2-(4,4,5,5-tetramethyl-1,3,2-dioxaborolan-2-yl)-1,3,2-dioxaborolane (1.82 g, 7.186 mmol, 2 equiv), Pd(dppf)Cl₂ (262.9 mg, 0.359 mmol, 0.1 equiv) and KOAc (1.06 g, 10.779 mmol, 3 equiv) in dioxane (16 mL) was stirred for overnight at 90° C. under nitrogen atmosphere. The resulting mixture was filtered, the filter cake was washed with dioxane (3×2 mL). The filtrate was concentrated under reduced pressure. The resulting mixture was diluted with water (50 mL). The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by reversed-phase flash chromatography to afford 2-benzyl-4-fluoro-7-(4,4,5,5-tetramethyl-1,3,2-dioxaborolan-2-yl)isoindoline (1.2 g, 95%) as a brown oil. LC-MS (ES, m/z)=354.05 [M+1]⁺.

Step 4. Preparation of 2-benzyl-7-fluoroisoindolin-4-ol. To a stirred mixture of 2-benzyl-4-fluoro-7-(4,4,5,5-tetramethyl-1,3,2-dioxaborolan-2-yl)isoindoline (1.2 g, 5.662 mmol, 1 equiv) in THF (10 mL) was added potassium peroxymonosulfate (5.22 g, 8.493 mmol, 1.5 equiv) in H₂O (10 mL) at 0° C. under air atmosphere. The resulting mixture was stirred for 2 h at room temperature under the air atmosphere. The reaction was quenched with sat. sodium hyposulfite (aq.) at 0° C. The resulting mixture was extracted with EtOAc (3×150 mL). The combined organic layers were washed with brine (3×100 mL), dried over anhydrous Na₂SO₄. After filtration, the filtrate was concentrated under reduced pressure. The residue was purified by reversed-phase flash chromatography to afford 2-benzyl-7-fluoroisoindolin-4-ol (1.1 g, 80%) as a dark green oil. LC-MS (ES, m/z)=243.95 [M+1]⁺.

Step 5. Preparation of tert-butyl 4-fluoro-7-hydroxyisoindoline-2-carboxylate. A mixture of 2-benzyl-7-fluoroisoindolin-4-ol (1.1 g, 4.522 mmol, 1 equiv) and Pd/C (2 g, Palladium 10% on Carbon (wetted with ca. 55% Water)) and Boc₂O (2.96 g, 13.566 mmol, 3 equiv) in MeOH (15 mL) was stirred for 2 h at room temperature under hydrogen atmosphere. The resulting mixture was filtered, the filter cake was washed with MeOH (3×5 mL). The filtrate was concentrated under reduced pressure. The residue was purified by silica gel column chromatography, eluted with PE/EA (1:1) to afford tert-butyl 4-fluoro-7-hydroxyisoindoline-2-carboxylate (630 mg, 55%) as an off-white solid. LC-MS (ES, m/z)=239.25 [M-t-Bu+ACN]⁺.

Step 6. Preparation of tert-butyl 4-(bromodifluoromethoxy)-7-fluoroisoindoline-2-carboxylate. A solution of tert-butyl 4-fluoro-7-hydroxyisoindoline-2-carboxylate (500 mg, 1.974 mmol, 1 equiv) in DMF (5 mL) was treated with NaH (94.75 mg, 3.948 mmol, 2 equiv) for 30 min at 0° C. under nitrogen atmosphere followed by the addition of dibromodifluoromethane (1.24 g, 5.922 mmol, 3 equiv) in portions at 0° C. The resulting mixture was stirred for 2 h at room temperature under nitrogen atmosphere. The reaction was quenched with sat. NH₄Cl (aq.) at 0° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reversed-phase flash chromatography to afford tert-butyl 4-(bromodifluoromethoxy)-7-fluoroisoindoline-2-carboxylate (280 mg, 37%) as a yellow solid. LC-MS (ES, m/z)=367.05 [M-t-Bu+ACN]⁺.

Step 7. Preparation of tert-butyl 4-fluoro-7-(trifluoromethoxy)isoindoline-2-carboxylate. A mixture of tert-butyl 4-(bromodifluoromethoxy)-7-fluoroisoindoline-2-carboxylate (270 mg, 0.706 mmol, 1 equiv) and AgBF₄ (1.38 g, 7.060 mmol, 10 equiv) in DCM (2.7 mL) was stirred for 2 h at room temperature under nitrogen atmosphere. The precipitated solids were collected by filtration and washed with DCM (3×5 mL). The resulting mixture was concentrated under reduced pressure. The residue was purified by reversed-phase flash chromatography to afford tert-butyl 4-fluoro-7-(trifluoromethoxy)isoindoline-2-carboxylate (200 mg, 44%) as a brown solid. LC-MS (ES, m/z)=222.1 [M-Boc+1]⁺.

Step 8. Preparation of 4-fluoro-7-(trifluoromethoxy)isoindoline. A mixture of tert-butyl 4-fluoro-7-(trifluoromethoxy)isoindoline-2-carboxylate (200 mg, 0.778 mmol, 1 equiv) in DCM (2.5 mL) was added TFA (0.5 mL) dropwise at 0° C. under air atmosphere. The resulting mixture was stirred for 2 h at room temperature under the air atmosphere. The resulting mixture was concentrated under vacuum to afford 4-fluoro-7-(trifluoromethoxy)isoindoline (150 mg, 87%) as a brown solid. The residue was used for the next step without further purification. LC-MS (ES, m/z)=222.1 [M+1]⁺.

Step 9. Preparation of 3-(5-(4-fluoro-7-(trifluoromethoxy)isoindoline-2-carbonyl)-1-oxoisoindolin-2-yl)piperidine-2,6-dione. A solution of 4-fluoro-7-(trifluoromethoxy)isoindoline (100 mg, 0.452 mmol, 1 equiv) and 2-(2,6-dioxopiperidin-3-yl)-1-oxo-3H-isoindole-5-carboxylic acid (156.4 mg, 0.542 mmol, 1.2 equiv) in ACN (2 mL) was treated with NMI (148.5 mg, 1.808 mmol, 4 equiv) at 0° C. under nitrogen atmosphere followed by the addition of TCFH (253.7 mg, 0.904 mmol, 2 equiv) in portions at 0° C. The resulting mixture was stirred for 2 h at room temperature. The resulting mixture was diluted with water and extracted with EtOAc (3×20 mL). The combined organic layers were washed with brine (3×30 mL), dried over anhydrous Na₂SO₄. After filtration, the filtrate was concentrated under reduced pressure. The residue was purified by reversed-phase flash chromatography to afford 3-(5-(4-fluoro-7-(trifluoromethoxy)isoindoline-2-carbonyl)-1-oxoisoindolin-2-yl)piperidine-2,6-dione (14 mg, 6%) as an off-white solid. LC-MS (ES, m/z)=490.1 [M+1]⁺. ¹H NMR (400 MHz, DMSO-d₆) δ 11.01 (s, 1H), 7.94-7.81 (m, 2H), 7.75 (dd, J=7.8, 1.4 Hz, 1H), 7.50-7.26 (m, 2H), 5.16 (dd, J=13.3, 5.1 Hz, 1H), 4.99 (d, J=9.6 Hz, 2H), 4.89 (d, J=18.1 Hz, 2H), 4.62-4.37 (m, 2H), 2.93 (ddd, J=17.1, 13.5, 5.5 Hz, 1H), 2.62 (ddd, J=17.2, 4.4, 2.2 Hz, 1H), 2.41 (td, J=13.1, 4.4 Hz, 1H), 2.11-1.96 (m, 1H).

### Example 186

### Synthesis of 3-(1-oxo-5-(7-oxa-2-azaspiro[3.5]nonane-2-carbonyl)isoindolin-2-yl)piperidine-2,6-dione

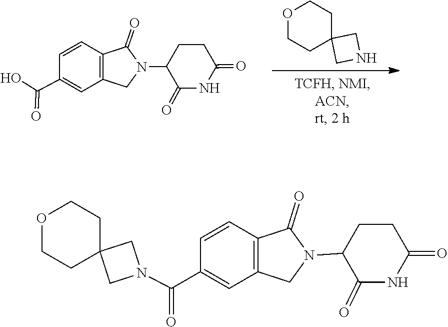 [SMILES: O=C1CCC(N2Cc3cc(C(=O)O)ccc3C2=O)C(=O)N1 ; C1CC2(CCO1)CNC2 ; O=C1CCC(N2Cc3cc(C(=O)N4CC5(CCOCC5)C4)ccc3C2=O)C(=O)N1]

Step 1. Preparation of 3-(1-oxo-5-(7-oxa-2-azaspiro[3.5]nonane-2-carbonyl)isoindolin-2-yl)piperidine-2,6-dione. To a stirred solution of 2-(2,6-dioxopiperidin-3-yl)-1-oxo-3H-isoindole-5-carboxylic acid (90 mg, 0.312 mmol, 1 equiv) and 7-oxa-2-azaspiro[3.5]nonane (59.57 mg, 0.468 mmol, 1.5 equiv) in ACN (1.5 mL) were added TCFH (175.20 mg, 0.624 mmol, 2 equiv) and NMI (102.54 mg, 1.248 mmol, 4 equiv) in portions at room temperature under air atmosphere. The resulting mixture was stirred for 2 h at room temperature under the air atmosphere. The resulting mixture was diluted with water and extracted with EA (3×30 mL). The combined organic layers were washed with brine (3×50 mL), dried over anhydrous Na₂SO₄. After filtration, the filtrate was concentrated under reduced pressure. The crude product (40 mg) was purified by Prep-HPLC to afford 3-(1-oxo-5-(7-oxa-2-azaspiro[3.5]nonane-2-carbonyl)isoindolin-2-yl)piperidine-2,6-dione (9.4 mg, 7.58%) as a white solid. LC-MS (ES, m/z)=398.25 [M+1]⁺. ¹H NMR (400 MHz, DMSO-d₆) δ 11.01 (s, 2H), 7.88 (s, 2H), 7.82-7.72 (m, 3H), 5.14 (dd, J=13.3, 5.1 Hz, 2H), 4.51 (d, J=17.6 Hz, 2H), 4.38 (d, J=17.6 Hz, 2H), 4.06 (s, 3H), 3.81 (s, 3H), 3.54 (q, J=5.7, 5.0 Hz, 4H), 3.47 (s, 3H), 3.17 (s, 1H), 2.92 (ddd, J=17.2, 13.6, 5.4 Hz, 2H), 2.66-2.57 (m, 2H), 2.48-2.33 (m, 3H), 2.03 (dtd, J=12.6, 5.3, 2.3 Hz, 2H), 1.71 (t, J=5.2 Hz, 6H), 1.24 (s, 1H).

### Example 187

### Synthesis of 3-(1-oxo-5-(6-oxa-2-azaspiro[3.4]octane-2-carbonyl)isoindolin-2-yl)piperidine-2,6-dione

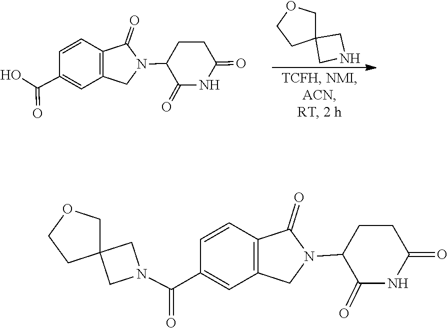 [SMILES: O=C1CCC(N2Cc3cc(C(=O)O)ccc3C2=O)C(=O)N1 ; C1CC2(CNC2)CO1 ; O=C1CCC(N2Cc3cc(C(=O)N4CC5(CCOC5)C4)ccc3C2=O)C(=O)N1]

Step 1. Preparation of 3-(1-oxo-5-(6-oxa-2-azaspiro[3.4]octane-2-carbonyl)isoindolin-2-yl)piperidine-2,6-dione. To a stirred solution of bis(6-oxa-2-azaspiro[3.4]octane); oxalic acid (153.7 mg, 0.486 mmol, 1.4 equiv) and 2-(2,6-dioxopiperidin-3-yl)-1-oxo-3H-isoindole-5-carboxylic acid (100 mg, 0.347 mmol, 1.00 equiv) in ACN (2 mL) was added NMI (113.9 mg, 1.388 mmol, 4 equiv) and TCFH (194.7 mg, 0.694 mmol, 2 equiv) at room temperature under nitrogen atmosphere. The resulting mixture was stirred for 2 h at room temperature under nitrogen atmosphere. The resulting mixture was diluted with water (50 mL). The resulting mixture was extracted with CHCl₃ (3×30 mL). The combined organic layers were washed with brine (3×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. The crude product (52 mg) was purified by Prep-HPLC to afford 3-(1-oxo-5-(6-oxa-2-azaspiro[3.4]octane-2-carbonyl)isoindolin-2-yl)piperidine-2,6-dione (9.0 mg, 6.73%) as a white solid. LC-MS (ES, m/z)=384 [M+1]⁺. ¹H NMR (400 MHz, DMSO-d₆) δ 11.01 (s, 1H), 7.87 (s, 1H), 7.82-7.68 (m, 2H), 5.13 (dd, J=13.3, 5.1 Hz, 1H), 4.50 (d, J=17.6 Hz, 1H), 4.44-4.25 (m, 3H), 4.05 (s, 2H), 3.86-3.62 (m, 4H), 2.92 (ddd, J=17.3, 13.6, 5.4 Hz, 1H), 2.61 (dd, J=17.0, 3.9 Hz, 1H), 2.46-2.29 (m, 1H), 2.13 (t, J=7.0 Hz, 2H), 2.08-1.95 (m, 1H).

### Example 188

### Synthesis of 3-(1-oxo-5-(2-oxa-6-azaspiro[3.3]heptane-6-carbonyl)isoindolin-2-yl)piperidine-2,6-dione

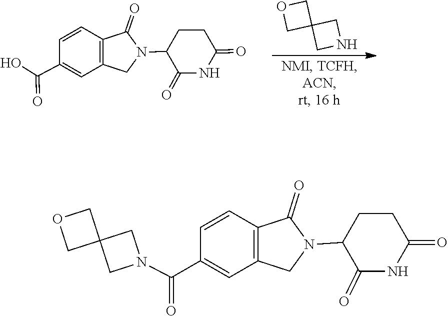 [SMILES: O=C1CCC(N2Cc3cc(C(=O)O)ccc3C2=O)C(=O)N1 ; C1NCC12COC2 ; O=C1CCC(N2Cc3cc(C(=O)N4CC5(COC5)C4)ccc3C2=O)C(=O)N1]

Step 1. Preparation of 3-(1-oxo-5-(2-oxa-6-azaspiro[3.3]heptane-6-carbonyl)isoindolin-2-yl)piperidine-2,6-dione. To a stirred solution of 2-(2,6-dioxopiperidin-3-yl)-1-oxo-3H-isoindole-5-carboxylic acid (100 mg, 0.347 mmol, 1 equiv) and NMI (85.4 mg, 1.041 mmol, 3 equiv) in ACN (5 mL) were added 2-oxa-6-azaspiro[3.3]heptane (51.5 mg, 0.520 mmol, 1.5 equiv) and TCFH (146.0 mg, 0.520 mmol, 1.5 equiv) in portions at room temperature. The resulting mixture was stirred for 16 h at room temperature. The resulting mixture was diluted with water (50 mL). The resulting mixture was extracted with EtOAc (3×30 mL). The combined organic layers were washed with brine (2×100 mL), dried over anhydrous Na₂SO₄. After filtration, the filtrate was concentrated under reduced pressure. The crude product (90 mg) was purified by Prep-HPLC to afford 3-(1-oxo-5-(2-oxa-6-azaspiro[3.3]heptane-6-carbonyl)isoindolin-2-yl)piperidine-2,6-dione (37 mg, 29%) as a white solid. LC-MS (ES, m/z)=370.2 [M+1]⁺. ¹H NMR (400 MHz, DMSO-d₆) (511.01 (s, 1H), 7.83 (s, 1H), 7.79 (d, J=7.9 Hz, 1H), 7.72 (dd, J=7.9, 1.4 Hz, 1H), 5.14 (dd, J=13.3, 5.1 Hz, 1H), 4.68 (q, J=6.7 Hz, 4H), 4.58-4.33 (m, 4H), 4.24 (s, 2H), 2.92 (ddd, J=17.2, 13.6, 5.4 Hz, 1H), 2.61 (ddd, J=17.3, 4.5, 2.2 Hz, 1H), 2.41 (qd, J=13.2, 4.4 Hz, 1H), 2.03 (ddd, J=9.6, 5.4, 2.7 Hz, 1H).

### Example 189

### Synthesis of 3-(5-(2,2-difluoro-6-azaspiro[3.4]octane-6-carbonyl)-1-oxoisoindolin-2-yl)piperidine-2,6-dione

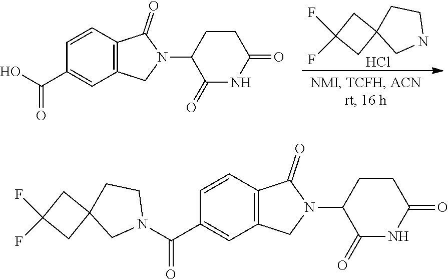

Step 1. Preparation of 3-(5-(2,2-difluoro-6-azaspiro[3.4]octane-6-carbonyl)-1-oxoisoindolin-2-yl)piperidine-2,6-dione. To a stirred solution of 2-(2,6-dioxopiperidin-3-yl)-1-oxo-3H-isoindole-5-carboxylic acid (100 mg, 0.347 mmol, 1 equiv) and NMI (85.4 mg, 1.041 mmol, 3 equiv) in ACN (5 mL) were added 2,2-difluoro-6-azaspiro[3.4]octane hydrochloride (76.4 mg, 0.416 mmol, 1.2 equiv) and TCFH (146.0 mg, 0.520 mmol, 1.5 equiv) in portions at room temperature. The resulting mixture was stirred for 16 h at room temperature. The resulting mixture was diluted with water (50 mL). The resulting mixture was extracted with EtOAc (3×30 mL). The combined organic layers were washed with brine (2×50 mL), dried over anhydrous Na₂SO₄. After filtration, the filtrate was concentrated under reduced pressure. The crude product was purified by Prep-HPLC to afford 3-(5-(2,2-difluoro-6-azaspiro[3.4]octane-6-carbonyl)-1-oxoisoindolin-2-yl)piperidine-2,6-dione (78 mg, 54%) as a white solid. LC-MS (ES, m/z)=418.1 [M+1]⁺. ¹H NMR (400 MHz, DMSO-d₆) δ 11.00 (s, 1H), 7.82-7.72 (m, 2H), 7.63 (ddd, J=7.7, 3.2, 1.4 Hz, 1H), 5.14 (ddd, J=13.4, 5.2, 2.3 Hz, 1H), 4.50 (dd, J=17.6, 3.5 Hz, 1H), 4.42-4.33 (m, 1H), 3.61-3.51 (m, 2H), 3.49-3.40 (m, 2H), 2.99-2.85 (m, 1H), 2.79-2.68 (m, 1H), 2.59 (td, J=14.7, 13.5, 8.5 Hz, 4H), 2.40 (td, J=13.2, 4.4 Hz, 1H), 1.99 (dt, J=22.8, 6.9 Hz, 3H).

### Example 190

### Synthesis of 3-(5-(8,8-difluoro-2-azaspiro[4.5]decane-2-carbonyl)-1-oxoisoindolin-2-yl)piperidine-2,6-dione

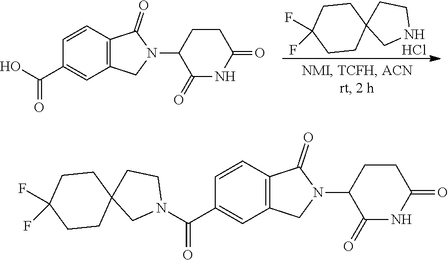

Step 1. Preparation of 3-(5-(8,8-difluoro-2-azaspiro[4.5]decane-2-carbonyl)-1-oxoisoindolin-2-yl)piperidine-2,6-dione. To a stirred solution of 8,8-difluoro-2-azaspiro[4.5]decane hydrochloride (85.9 mg, 0.406 mmol, 1.3 equiv) and 2-(2,6-dioxopiperidin-3-yl)-1-oxo-3H-isoindole-5-carboxylic acid (90 mg, 0.312 mmol, 1.00 equiv) in ACN (2 mL) was added NMI (102.5 mg, 1.248 mmol, 4 equiv) and TCFH (175.2 mg, 0.624 mmol, 2 equiv) at room temperature under nitrogen atmosphere. The resulting mixture was stirred for 2 h at room temperature under nitrogen atmosphere. The resulting mixture was diluted with water (30 mL). The resulting mixture was extracted with EtOAc (3×20 mL). The combined organic layers were washed with brine (3×50 mL), dried over anhydrous Na₂SO₄. After filtration, the filtrate was concentrated under reduced pressure. The residue was purified by reversed-phase flash chromatography to afford 3-(5-(8,8-difluoro-2-azaspiro[4.5]decane-2-carbonyl)-1-oxoisoindolin-2-yl)piperidine-2,6-dione (85 mg, 58%) as light pink solid. LC-MS (ES, m/z)=446.15 [M+1]⁺.

¹H NMR (400 MHz, DMSO-d₆) δ 11.01 (s, 1H), 7.82-7.57 (m, 3H), 5.18-5.09 (m, 1H), 4.55-4.32 (m, 2H), 3.58 (t, J=7.3 Hz, 1H), 3.46 (t, J=6.9 Hz, 1H), 2.97-2.83 (m, 2H), 2.68-2.57 (m, 1H), 2.40 (td, J=13.2, 4.4 Hz, 1H), 2.05-1.89 (m, 4H), 1.81 (dt, J=22.3, 7.3 Hz, 4H), 1.66 (dp, J=20.1, 6.9, 6.2 Hz, 2H), 1.59-1.49 (m, 2H).

### Example 191

### Synthesis of 3-(5-(3-fluoro-3-(3-fluorophenyl)azetidine-1-carbonyl)-1-oxoisoindolin-2-yl)piperidine-2,6-dione

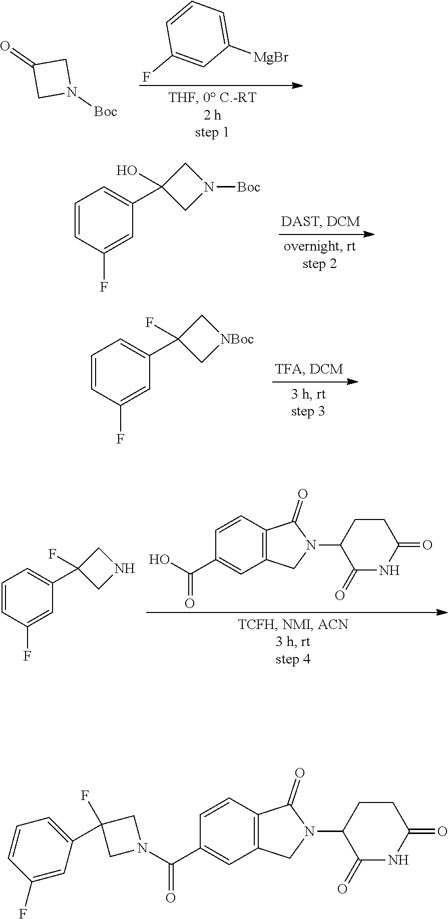

Step 1. Preparation of tert-butyl 3-(3-fluorophenyl)-3-hydroxyazetidine-1-carboxylate. To a stirred mixture of tert-butyl 3-oxoazetidine-1-carboxylate (5 g, 29.206 mmol, 1 equiv) in THF (100 mL) was added bromo(3-fluorophenyl)magnesium (43.5 mL, 1.0M, 1.49 equiv) dropwise at 0° C. under nitrogen atmosphere. The resulting mixture was stirred for 2 h at room temperature under nitrogen atmosphere. The reaction was quenched with NH₄Cl (aq) at 0° C. and extracted with EtOAc (3×200 mL). The combined organic layers were washed with brine (2×500 mL), dried over anhydrous Na₂SO₄. After filtration, the filtrate was concentrated under reduced pressure. The residue was purified by reversed-phase flash chromatography to afford tert-butyl 3-(3-fluorophenyl)-3-hydroxyazetidine-1-carboxylate (6 g, 77%) as yellow oil. LC-MS (ES, m/z)=253.10 [M-t-Bu+ACN+1]⁺.

Step 2. Preparation of tert-butyl 3-fluoro-3-(3-fluorophenyl)azetidine-1-carboxylate. To a stirred mixture of tert-butyl 3-(3-fluorophenyl)-3-hydroxyazetidine-1-carboxylate (1 g, 3.741 mmol, 1 equiv) in DCM (15 mL) were added DAST (1.2 g, 7.482 mmol, 2 equiv) at 0° C. under nitrogen atmosphere. The resulting mixture was stirred overnight at room temperature. The reaction was quenched with NH₄Cl (aq) at 0° C. and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 3-fluoro-3-(3-fluorophenyl)azetidine-1-carboxylate (400 mg, 40%) as yellow oil. LC-MS (ES, m/z)=253.10 [M-t-Bu+ACN+1]⁺.

Step 3. Preparation of 3-fluoro-3-(3-fluorophenyl)azetidine. To a stirred mixture of tert-butyl 3-fluoro-3-(3-fluorophenyl)azetidine-1-carboxylate (300 mg, 1.114 mmol, 1 equiv) in DCM (3 mL) was added TFA (0.6 mL) dropwise at 0° C. The resulting mixture was stirred for 3 h at room temperature. The resulting mixture was concentrated under reduced pressure to afford 3-fluoro-3-(3-fluorophenyl)azetidine (100 mg, 53%) as yellow oil. The crude product was used in the next step directly without further purification. LC-MS (ES, m/z)=170.10 [M+1].

Step 4. Preparation of 3-(5-(3-fluoro-3-(3-fluorophenyl)azetidine-1-carbonyl)-1-oxoisoindolin-2-yl)piperidine-2,6-dione. To a stirred mixture of 3-fluoro-3-(3-fluorophenyl)azetidine (117.3 mg, 0.694 mmol, 2 equiv) and 2-(2,6-dioxopiperidin-3-yl)-1-oxo-3H-isoindole-5-carboxylic acid (100 mg, 0.347 mmol, 1.00 equiv) in ACN (3 mL) were added TCFH (146.0 mg, 0.520 mmol, 1.5 equiv) and NMI (113.9 mg, 1.388 mmol, 4 equiv) at room temperature. The resulting mixture was stirred for 3 h at room temperature. The resulting mixture was diluted with water (30 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(3-fluoro-3-(3-fluorophenyl)azetidine-1-carbonyl)-1-oxoisoindolin-2-yl)piperidine-2,6-dione (34 mg, 22%) as white solid. LC-MS (ES, m/z)=440.0 [M+1]⁺. ¹H NMR (400 MHz, DMSO-d₆) δ 11.01 (s, 1H), 7.97 (s, 1H), 7.89-7.77 (m, 2H), 7.58-7.49 (m, 1H), 7.49-7.40 (m, 2H), 7.33-7.24 (m, 1H), 5.14 (dd, J=13.3, 5.1 Hz, 1H), 4.81 (s, 1H), 4.60-4.47 (m, 3H), 4.39 (d, J=17.6 Hz, 1H), 2.96-2.87 (m, 1H), 2.61 (d, J=17.3 Hz, 1H), 2.42 (dd, J=13.0, 4.4 Hz, 1H), 2.07-1.99 (m, 1H).

### Example 192

### Synthesis of 3-(5-(3-(4-fluorophenyl)azetidine-1-carbonyl)-1-oxoisoindolin-2-yl)piperidine-2,6-dione

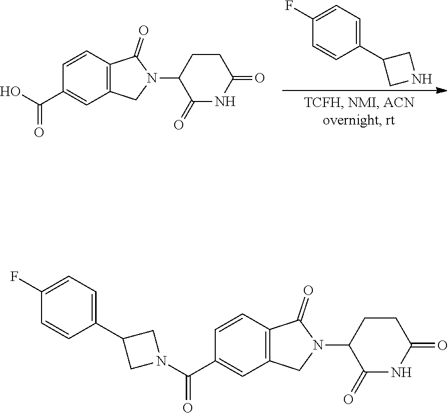 [SMILES: O=C1CCC(N2Cc3cc(C(=O)O)ccc3C2=O)C(=O)N1 ; Fc1ccc(C2CNC2)cc1 ; O=C1CCC(N2Cc3cc(C(=O)N4CC(c5ccc(F)cc5)C4)ccc3C2=O)C(=O)N1]

Step 1. Preparation of 3-(5-(3-(4-fluorophenyl)azetidine-1-carbonyl)-1-oxoisoindolin-2-yl)piperidine-2,6-dione. To a stirred mixture of 3-(4-fluorophenyl)azetidine (39.3 mg, 0.259 mmol, 1.5 equiv) and 2-(2,6-dioxopiperidin-3-yl)-1-oxo-3H-isoindole-5-carboxylic acid (50 mg, 0.173 mmol, 1.00 equiv) in ACN (1.5 mL) were added TCFH (73.0 mg, 0.259 mmol, 1.5 equiv) and NMI (56.9 mg, 0.692 mmol, 4 equiv) at room temperature. The resulting mixture was stirred overnight at room temperature. The resulting mixture was diluted with water (30 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(3-(4-fluorophenyl)azetidine-1-carbonyl)-1-oxoisoindolin-2-yl)piperidine-2,6-dione (11 mg, 15%) as white solid. LC-MS (ES, m/z)=422.10 [M+1]⁺. ¹H NMR (400 MHz, DMSO-d₆) δ 11.01 (s, 1H), 7.92 (s, 1H), 7.80 (s, 2H), 7.51-7.43 (m, 2H), 7.20 (t, J=8.9 Hz, 2H), 5.14 (dd, J=13.3, 5.1 Hz, 1H), 4.66 (t, J=8.9 Hz, 1H), 4.56-4.46 (m, 2H), 4.37 (dd, J=16.6, 9.6 Hz, 2H), 4.04-3.96 (m, 2H), 2.96-2.86 (m, 1H), 2.61 (ddd, J=17.5, 4.6, 2.4 Hz, 1H), 2.40 (td, J=13.2, 4.4 Hz, 1H), 2.10-1.99 (m, 1H).

### Example 193

### Synthesis of 3-(5-(3-(3-fluorophenyl)azetidine-1-carbonyl)-1-oxoisoindolin-2-yl)piperidine-2,6-dione

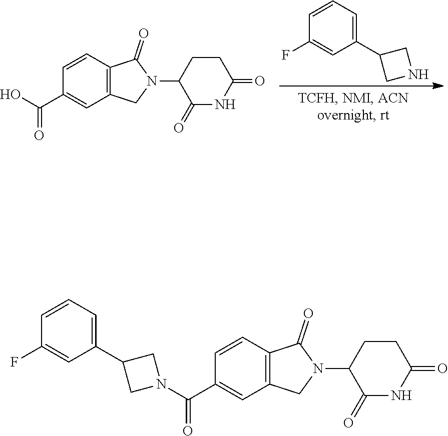

Step 1. Preparation of 3-(5-(3-(3-fluorophenyl)azetidine-1-carbonyl)-1-oxoisoindolin-2-yl)piperidine-2,6-dione. To a stirred mixture of 3-(3-fluorophenyl)azetidine (39.3 mg, 0.259 mmol, 1.5 equiv) and 2-(2,6-dioxopiperidin-3-yl)-1-oxo-3H-isoindole-5-carboxylic acid (50 mg, 0.173 mmol, 1.00 equiv) in ACN (1 mL) was added TCFH (73.0 mg, 0.259 mmol, 1.5 equiv) and NMI (56.9 mg, 0.692 mmol, 4 equiv) at room temperature. The resulting mixture was stirred overnight at room temperature. The resulting mixture was diluted with water (20 mL) and extracted with EtOAc (3×10 mL). The combined organic layers were washed with brine (2×30 mL), dried over anhydrous Na₂SO₄. After filtration, the filtrate was concentrated under reduced pressure. The residue was purified by reversed-phase flash chromatography to afford 3-(5-(3-(3-fluorophenyl)azetidine-1-carbonyl)-1-oxoisoindolin-2-yl)piperidine-2,6-dione (8.4 mg, 11.49%) as white solid. LC-MS (ES, m/z)=420.15 [M−1]⁻. ¹H NMR (400 MHz, DMSO-d₆) δ 11.01 (s, 1H), 7.92 (s, 1H), 7.81 (d, J=1.7 Hz, 2H), 7.41 (td, J=8.0, 6.1 Hz, 1H), 7.31 (dt, J=10.4, 2.2 Hz, 1H), 7.25 (d, J=7.8 Hz, 1H), 7.10 (td, J=8.6, 2.6 Hz, 1H), 5.14 (dd, J=13.3, 5.1 Hz, 1H), 4.65 (t, J=8.7 Hz, 1H), 4.56-4.47 (m, 2H), 4.40 (q, J=10.5, 9.4 Hz, 2H), 4.11-3.95 (m, 2H), 2.89 (d, J=7.6 Hz, 1H), 2.65-2.58 (m, 1H), 2.41 (dd, J=13.2, 4.4 Hz, 1H), 2.06-2.00 (m, 1H).

### Example 194

### Synthesis of 3-(5-(3-(2-fluorophenyl)azetidine-1-carbonyl)-1-oxoisoindolin-2-yl)piperidine-2,6-dione

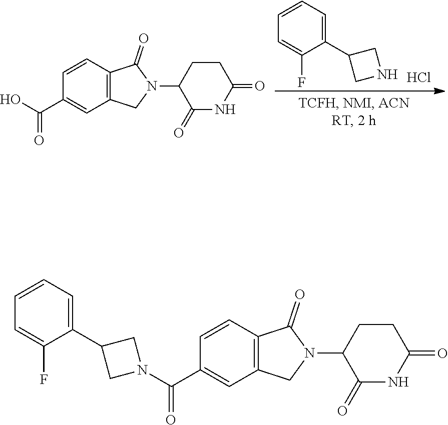

Step 1. Preparation of 3-(5-(3-(2-fluorophenyl)azetidine-1-carbonyl)-1-oxoisoindolin-2-yl)piperidine-2,6-dione. To a stirred solution of 3-(2-fluorophenyl)azetidine hydrochloride (76.2 mg, 0.406 mmol, 1.3 equiv) and 2-(2,6-dioxopiperidin-3-yl)-1-oxo-3H-isoindole-5-carboxylic acid (90 mg, 0.312 mmol, 1.00 equiv) in ACN (2 mL) was added NMI (102.6 mg, 1.248 mmol, 4 equiv) and TCFH (175.2 mg, 0.624 mmol, 2 equiv) at room temperature under nitrogen atmosphere. The resulting mixture was stirred for 2 h at room temperature under nitrogen atmosphere. The resulting mixture was diluted with water (30 mL). The resulting mixture was extracted with EtOAc (3×20 mL). The combined organic layers were washed with brine (3×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. The resulting mixture was concentrated under vacuum. The crude product (60 mg) was purified by Prep-HPLC to afford 3-(5-(3-(2-fluorophenyl)azetidine-1-carbonyl)-1-oxoisoindolin-2-yl)piperidine-2,6-dione (8.0 mg, 5.64%) as white solid. LC-MS (ES, m/z)=422.15 [M+1]⁺. ¹H NMR (400 MHz, DMSO-d₆) δ 10.99 (s, 1H), 7.91 (s, 1H), 7.80 (s, 2H), 7.65-7.50 (m, 1H), 7.34 (tdd, J=7.5, 5.3, 1.8 Hz, 1H), 7.27-7.13 (m, 2H), 5.14 (dd, J=13.3, 5.1 Hz, 1H), 4.80-4.59 (m, 1H), 4.58-4.33 (m, 4H), 4.21-4.06 (m, 2H), 2.92 (ddd, J=17.3, 13.6, 5.4 Hz, 2H), 2.61 (ddd, J=17.2, 4.6, 2.3 Hz, 1H), 2.40 (td, J=13.2, 4.5 Hz, 1H), 2.04 (dtt, J=15.2, 5.4, 2.4 Hz, 1H).

### Example 195

### Synthesis of 3-(5-(6-(3-fluorophenyl)-2-azaspiro[3.3]heptane-2-carbonyl)-1-oxoisoindolin-2-yl)piperidine-2,6-dione

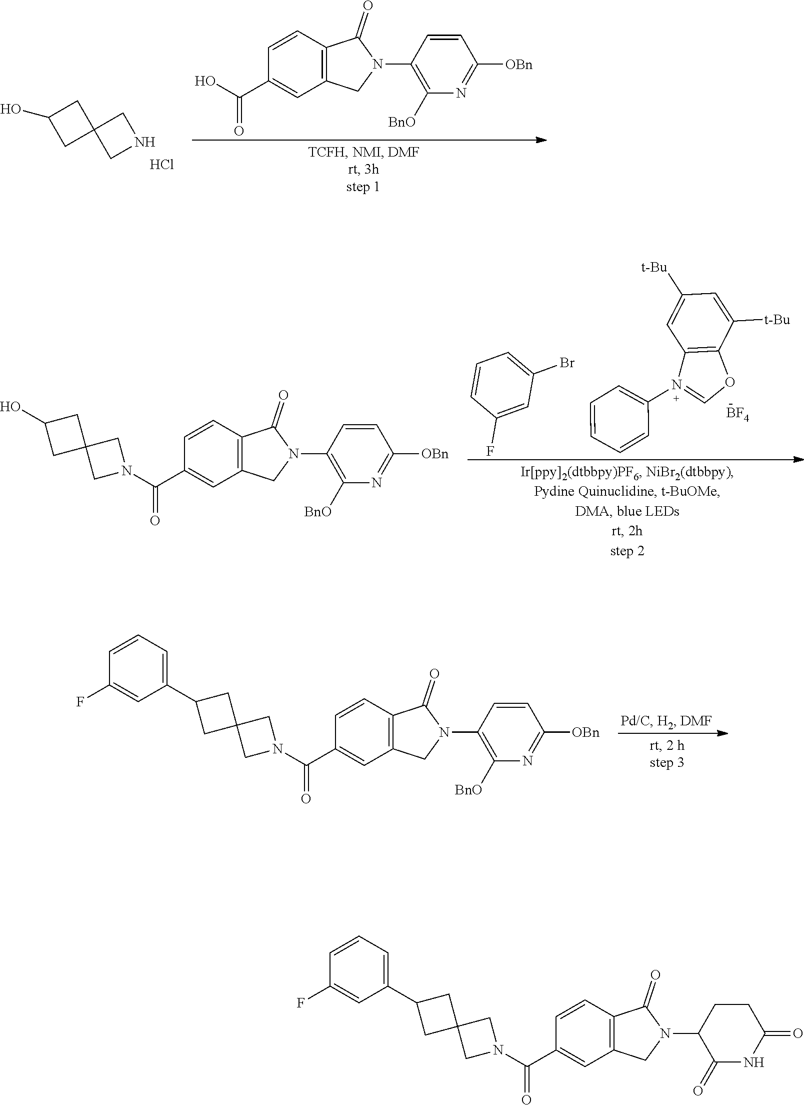

Step 1. Preparation of 2-(2,6-bis(benzyloxy)pyridin-3-yl)-5-(6-hydroxy-2-azaspiro[3.3]heptane-2-carbonyl)isoindolin-1-one. To a stirred mixture of 2-[2,6-bis(benzyloxy)pyridin-3-yl]-1-oxo-3H-isoindole-5-carboxylic acid (500 mg, 1.072 mmol, 1.00 equiv) and 2-azaspiro[3.3]heptan-6-ol hydrochloride (160.4 mg, 1.072 mmol, 1.00 equiv) in DMF (10 mL) were added NMI (352.0 mg, 4.288 mmol, 4 equiv) and TCFH (451.1 mg, 1.608 mmol, 1.5 equiv) at room temperature under air atmosphere. The resulting mixture was stirred for 3 h at room temperature under air atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(6-hydroxy-2-azaspiro[3.3]heptane-2-carbonyl)isoindolin-1-one (500 mg, 83%) as yellow solid. LC-MS (ES, m/z)=562.35 [M+1]⁺.

Step 2. Preparation of 2-(2,6-bis(benzyloxy)pyridin-3-yl)-5-(6-(3-fluorophenyl)-2-azaspiro[3.3]heptane-2-carbonyl)isoindolin-1-one. To a stirred mixture of 2-(2,6-bis(benzyloxy)pyridin-3-yl)-5-(6-hydroxy-2-azaspiro[3.3]heptane-2-carbonyl)isoindolin-1-one (300 mg, 0.534 mmol, 1 equiv) and 5,7-di-tert-butyl-3-phenylbenzo[d]oxazol-3-ium tetrafluoroborate (337.8 mg, 0.855 mmol, 1.60 equiv) in 2-methoxy-2-methylpropane (5 mL) was added pyridine (67.6 mg, 0.855 mmol, 1.60 equiv) at 0° C. under nitrogen atmosphere. The resulting mixture was stirred for 10 min at 0° C. under nitrogen atmosphere. To the above mixture was added a mixture of m-bromofluorobenzene (140.2 mg, 0.801 mmol, 1.50 equiv), [Ir(dtbbpy)(ppy)₂][PF₆] (4.9 mg, 0.005 mmol, 0.01 equiv), NiBr₂(dtbbpy) (13.0 mg, 0.027 mmol, 0.05 equiv) and 1-azabicyclo[2.2.2]octane (103.9 mg, 0.934 mmol, 1.75 equiv) in DMA (5 mL) at room temperature under nitrogen atmosphere. The resulting mixture was stirred for 2 h at room temperature under blue LEDs atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(6-(3-fluorophenyl)-2-azaspiro[3.3]heptane-2-carbonyl)isoindolin-1-one (230 mg, 67%) as yellow solid. LC-MS (ES, m/z)=640.45 [M+1]⁺.

Step 3. Preparation of 3-(5-(6-(3-fluorophenyl)-2-azaspiro[3.3]heptane-2-carbonyl)-1-oxoisoindolin-2-yl)piperidine-2,6-dione. To a solution of 2-(2,6-bis(benzyloxy)pyridin-3-yl)-5-(6-(3-fluorophenyl)-2-azaspiro[3.3]heptane-2-carbonyl)isoindolin-1-one (230 mg, 0.360 mmol, 1 equiv) in dimethylformamide (4 mL) was added Pd/C (100 mg, Palladium 10% on Carbon (wetted with ca. 55% Water) under nitrogen atmosphere in a 10 mL round-bottom flask. The mixture was hydrogenated at room temperature for 2 h under hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford 3-(5-(6-(3-fluorophenyl)-2-azaspiro[3.3]heptane-2-carbonyl)-1-oxoisoindolin-2-yl)piperidine-2,6-dione (38 mg, 23%) as an off-white solid. LC-MS (ES, m/z)=462.25 [M+1]⁺. ¹H NMR (400 MHz, DMSO-d₆) δ 11.01 (s, 1H), 7.93-7.69 (m, 3H), 7.33 (dt, J=8.9, 7.0 Hz, 1H), 7.02 (dd, J=25.7, 8.2 Hz, 3H), 5.14 (d, J=12.7 Hz, 1H), 4.56-4.33 (m, 3H), 4.24 (d, J=5.4 Hz, 2H), 4.02 (s, 1H), 3.52-3.35 (m, 1H), 3.00-2.85 (m, 1H), 2.61 (d, J=15.4 Hz, 3H), 2.45-2.22 (m, 3H), 2.03 (d, J=12.3 Hz, 1H), 1.36 (s, 1H).

### Example 196

### Synthesis of 3-(5-(6-(2-fluorophenyl)-2-azaspiro[3.3]heptane-2-carbonyl)-1-oxoisoindolin-2-yl)piperidine-2,6-dione

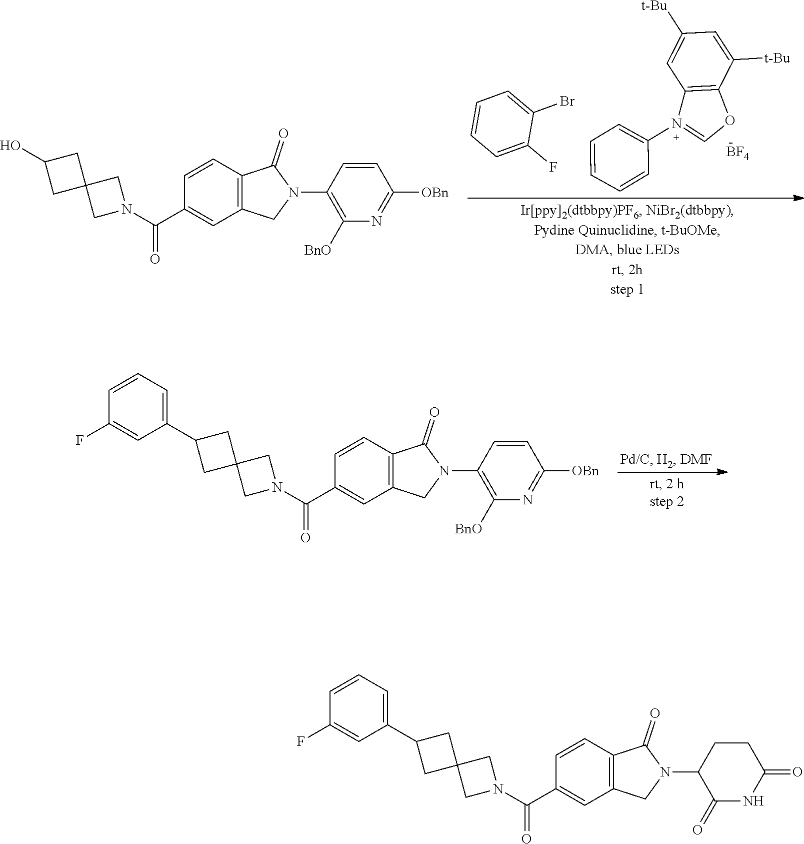

Step 1. Preparation of 2-(2,6-bis(benzyloxy)pyridin-3-yl)-5-(6-(2-fluorophenyl)-2-azaspiro[3.3]heptane-2-carbonyl)isoindolin-1-one. To a stirred mixture of 2-[2,6-bis(benzyloxy)pyridin-3-yl]-5-{6-hydroxy-2-azaspiro[3.3]heptane-2-carbonyl}-3H-isoindol-1-one (300 mg, 0.534 mmol, 1 equiv) and 5,7-di-tert-butyl-3-phenylbenzo[d]oxazol-3-ium tetrafluoroborate (337.8 mg, 0.855 mmol, 1.60 equiv) in 2-methoxy-2-methylpropane (5 mL) was added pyridine (67.6 mg, 0.855 mmol, 1.60 equiv). The resulting mixture was stirred for 10 min at 0° C. under nitrogen atmosphere. To the above mixture was added a mixture of 1-bromo-2-fluorobenzene (140.2 mg, 0.801 mmol, 1.50 equiv), [Ir(dtbbpy)(ppy)₂][PF₆] (4.9 mg, 0.005 mmol, 0.01 equiv), NiBr₂(dtbbpy) (13.0 mg, 0.027 mmol, 0.05 equiv) and 1-azabicyclo[2.2.2]octane (103.9 mg, 0.934 mmol, 1.75 equiv) in DMA (5 mL) at room temperature under nitrogen atmosphere. The resulting mixture was stirred for 2 h at room temperature under blue LEDs atmosphere. The resulting mixture was diluted with water (50 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(6-(2-fluorophenyl)-2-azaspiro[3.3]heptane-2-carbonyl)isoindolin-1-one (200 mg, crude) as a yellow solid. LC-MS (ES, m/z)=640.35 [M+1].

Step 2. Preparation of 3-(5-(6-(2-fluorophenyl)-2-azaspiro[3.3]heptane-2-carbonyl)-1-oxoisoindolin-2-yl)piperidine-2,6-dione. To a solution of 2-(2,6-bis(benzyloxy)pyridin-3-yl)-5-(6-(2-fluorophenyl)-2-azaspiro[3.3]heptane-2-carbonyl)isoindolin-1-one (200 mg, 0.313 mmol, 1 equiv) in dimethylformamide (5 mL) was added Pd/C (100 mg, Palladium 10% on Carbon (wetted with ca. 55% Water) under nitrogen atmosphere in a 25 mL round-bottom flask. The mixture was hydrogenated at room temperature for 2 h under hydrogen atmosphere using a hydrogen balloon, The resulting mixture was filtered through a Celite pad, and the filter cake was washed with THF (3×5 mL). The filtrate was concentrated under reduced pressure. The residue was purified by reversed-phase flash chromatography to afford 3-(5-(6-(2-fluorophenyl)-2-azaspiro[3.3]heptane-2-carbonyl)-1-oxoisoindolin-2-yl)piperidine-2,6-dione (31 mg, 21%) as an off-white solid. LC-MS (ES, m/z)=462.25 [M+1]⁺. ¹H NMR (400 MHz, DMSO-d₆) δ 11.00 (s, 1H), 7.87 (d, J=11.9 Hz, 1H), 7.77 (p, J=7.9 Hz, 2H), 7.39-7.28 (m, 1H), 7.27-7.20 (m, 1H), 7.20-7.07 (m, 2H), 5.14 (dt, J=13.3, 4.5 Hz, 1H), 4.56-4.46 (m, 2H), 4.38 (dd, J=17.5, 6.8 Hz, 1H), 4.25 (s, 2H), 4.02 (s, 1H), 3.55 (dp, J=26.8, 8.8 Hz, 1H), 2.99-2.85 (m, 1H), 2.61 (d, J=13.7 Hz, 3H), 2.47-2.28 (m, 3H), 2.04 (s, 1H).

### Example 197

### Synthesis of 3-(6-(4-chloro-5-fluoroisoindoline-2-carbonyl)benzo[d]oxazol-2-yl)piperidine-2,6-dione

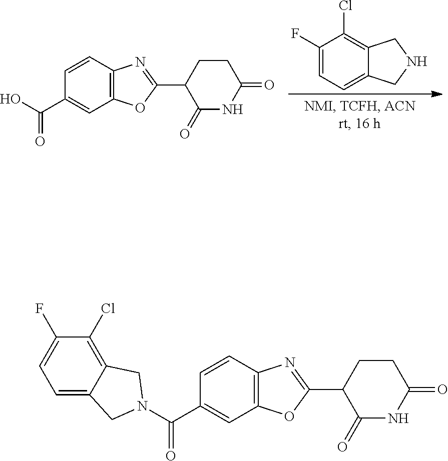 [SMILES: O=C1CCC(c2nc3ccc(C(=O)O)cc3o2)C(=O)N1 ; Fc1ccc2c(c1Cl)CNC2 ; O=C1CCC(c2nc3ccc(C(=O)N4Cc5ccc(F)c(Cl)c5C4)cc3o2)C(=O)N1]

Step 1. Preparation of 3-(6-(4-chloro-5-fluoroisoindoline-2-carbonyl)benzo[d]oxazol-2-yl)piperidine-2,6-dione. To a stirred mixture of 2-(2,6-dioxopiperidin-3-yl)-1,3-benzoxazole-6-carboxylic acid (100 mg, 0.365 mmol, 1 equiv) and NMI (119.7 mg, 1.460 mmol, 4 equiv) in ACN (2 mL) were added 4-chloro-5-fluoro-2,3-dihydro-1H-isoindole (75.0 mg, 0.438 mmol, 1.2 equiv) and TCFH (153.4 mg, 0.547 mmol, 1.5 equiv) at room temperature. The resulting mixture was stirred for 16 h at room temperature. The reaction was diluted with water (30 mL) at 0° C. and extracted with EtOAc (3×20 mL). The combined organic layers were washed with brine (2×50 mL), dried over anhydrous Na₂SO₄. After filtration, the filtrate was concentrated under reduced pressure. The crude product (65 mg) was purified by Prep-HPLC to afford 3-(6-(4-chloro-5-fluoroisoindoline-2-carbonyl)benzo[d]oxazol-2-yl)piperidine-2,6-dione (12 mg, 7.88%) as a white solid. LC-MS (ES, m/z)=428.10 [M+1]⁺. ¹H NMR (400 MHz, DMSO-d₆) δ 11.16 (s, 1H), 8.07 (d, J=1.8 Hz, 1H), 7.86 (dd, J=8.2, 2.8 Hz, 1H), 7.64 (td, J=7.9, 1.5 Hz, 1H), 7.48-7.26 (m, 2H), 4.94 (d, J=10.0 Hz, 2H), 4.87 (s, 2H), 4.57 (dd, J=11.3, 5.0 Hz, 1H), 2.81-2.61 (m, 2H), 2.49-2.29 (m, 2H).

### Example 198

### Synthesis of 3-(6-(6,6-difluoro-2-azaspiro[3.4]octane-2-carbonyl)benzo[d]oxazol-2-yl)piperidine-2,6-dione

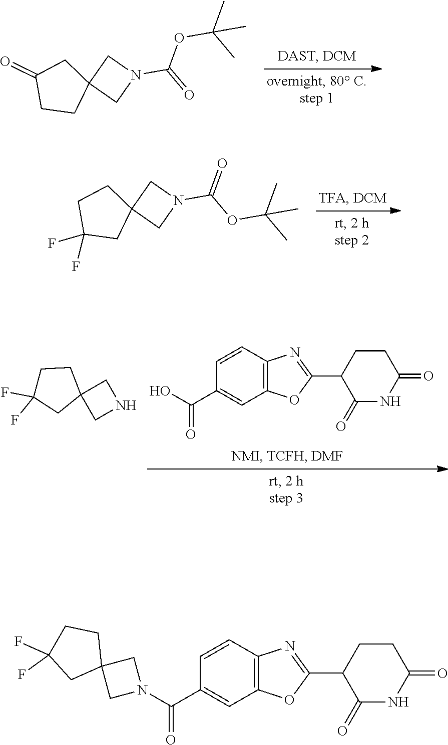 [SMILES: CC(C)(C)OC(=O)N1CC2(CCC(=O)C2)C1 ; CC(C)(C)OC(=O)N1CC2(CCC(F)(F)C2)C1 ; FC1(F)CCC2(CNC2)C1 ; O=C1CCC(c2nc3ccc(C(=O)O)cc3o2)C(=O)N1 ; O=C1CCC(c2nc3ccc(C(=O)N4CC5(CCC(F)(F)C5)C4)cc3o2)C(=O)N1]

Step 1. Preparation of tert-butyl 6,6-difluoro-2-azaspiro[3.4]octane-2-carboxylate. A mixture of tert-butyl 6-oxo-2-azaspiro[3.4]octane-2-carboxylate (1 g, 4.439 mmol, 1 equiv) and BAST (7.86 g, 35.512 mmol, 8 equiv) in CHCl₃ (10 mL) was stirred overnight at 80° C. under nitrogen atmosphere. The reaction was quenched by the addition of sat. NH₄Cl (aq.) (5 mL) at 0° C. and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6,6-difluoro-2-azaspiro[3.4]octane-2-carboxylate (730 mg, 67%) as a brown oil. LC-MS (ES, m/z)=233.15 [M-t-Bu+ACN+1]⁺.

Step 2. Preparation of 6,6-difluoro-2-azaspiro[3.4]octane. A mixture of tert-butyl 6,6-difluoro-2-azaspiro[3.4]octane-2-carboxylate (350 mg, 1.415 mmol, 1 equiv) in DCM (3 mL) was added TFA (0.6 mL) dropwise and stirred for 2 h at room temperature under the air atmosphere. The resulting mixture was concentrated under vacuum to afford 6,6-difluoro-2-azaspiro[3.4]octane (190 mg, crude) as a brown oil. The crude product was used in the next step directly without further purification. LC-MS (ES, m/z)=148.15 [M+1]⁺.

Step 3. Preparation of 3-(6-(6,6-difluoro-2-azaspiro[3.4]octane-2-carbonyl)benzo[d]oxazol-2-yl)piperidine-2,6-dione. A mixture of 6,6-difluoro-2-azaspiro[3.4]octane (83.72 mg, 0.569 mmol, 1.2 equiv), 2-(2,6-dioxopiperidin-3-yl)-1,3-benzoxazole-6-carboxylic acid (130 mg, 0.474 mmol, 1.00 equiv), NMI (77.8 mg, 0.948 mmol, 2 equiv) and TCFH (532.0 mg, 1.896 mmol, 4 equiv) in DMF (1.5 mL) was stirred for 2 h at room temperature under air atmosphere. The reaction was diluted with water (30 mL) at 0° C. and extracted with EtOAc (3×20 mL). The combined organic layers were washed with brine (2×50 mL), dried over anhydrous Na₂SO₄. After filtration, the filtrate was concentrated under reduced pressure. The crude product was purified by Prep-HPLC to afford 3-(6-(6,6-difluoro-2-azaspiro[3.4]octane-2-carbonyl)benzo[d]oxazol-2-yl)piperidine-2,6-dione (93 mg, 48%) as off-white solid. LC-MS (ES, m/z)=404.20 [M+1]⁺. ¹H NMR (400 MHz, DMSO-d₆) δ 11.15 (s, 1H), 8.00 (d, J=1.4 Hz, 1H), 7.81 (d, J=8.3 Hz, 1H), 7.67 (dd, J=8.3, 1.5 Hz, 1H), 4.56 (dd, J=11.3, 5.1 Hz, 1H), 4.35-4.22 (m, 2H), 4.09-3.94 (m, 2H), 2.79-2.59 (m, 2H), 2.48-2.38 (m, 3H), 2.37-2.30 (m, 1H), 2.15 (dt, J=20.9, 7.9 Hz, 3H), 2.08-2.01 (m, 2H).

### Example 199

### Synthesis of 3-(6-(4-fluoro-6-methylisoindoline-2-carbonyl)benzo[d]oxazol-2-yl)piperidine-2,6-dione

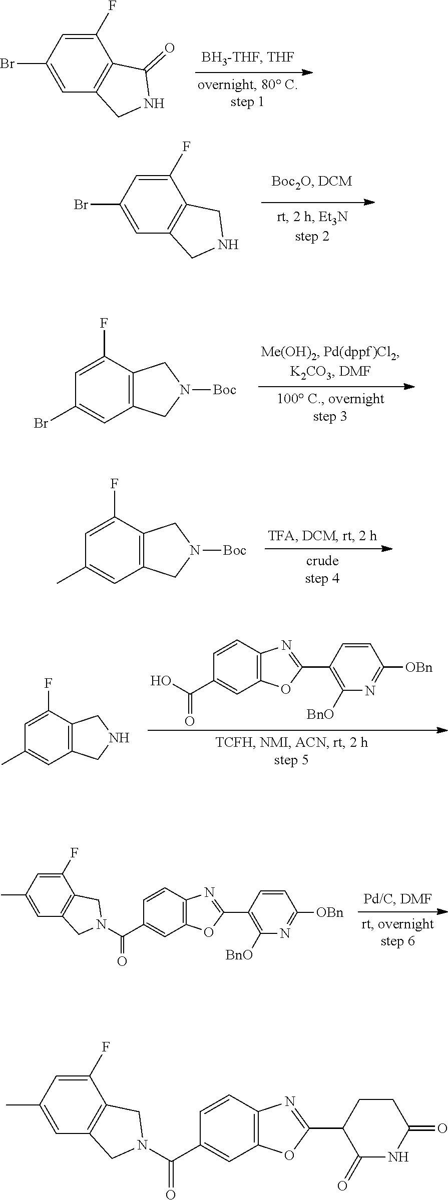

Step 1. Preparation of 6-bromo-4-fluoroisoindoline. A mixture of 5-bromo-7-fluoro-2,3-dihydroisoindol-1-one (2.5 g, 10.868 mmol, 1 equiv) and BH₃-THF (32 mL, 1.0M) in THF (15 mL) was stirred overnight at 80° C. under nitrogen atmosphere. The reaction was quenched with Water at 0° C. The residue was acidified with HCl (aq.) and extracted with DCM (3×100 mL). The combined organic layers were washed with brine (3×200 mL), dried over anhydrous Na₂SO₄. After filtration, the filtrate was concentrated under reduced pressure. The residue was purified by reversed-phase flash chromatography to afford 6-bromo-4-fluoroisoindoline (1.2 g, 51%) as a pink solid. LC-MS (ES, m/z)=216.0 [M+1]⁺.

Step 2. Preparation of tert-butyl 6-bromo-4-fluoroisoindoline-2-carboxylate. A mixture of 6-bromo-4-fluoroisoindoline (1.2 g, 5.554 mmol, 1 equiv), Et₃N (1.69 g, 16.662 mmol, 3 equiv) and Boc₂O (2.42 g, 11.108 mmol, 2 equiv) in DCM (18 mL) was stirred for 2 h at room temperature under air atmosphere. The reaction was diluted with water and extracted with DCM (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 6-bromo-4-fluoroisoindoline-2-carboxylate (1.39 g, 79%) as a yellow oil. LC-MS (ES, m/z)=301.0 [M+1]⁺.

Step 3. Preparation of tert-butyl 4-fluoro-6-methylisoindoline-2-carboxylate. A mixture of tert-butyl 6-bromo-4-fluoroisoindoline-2-carboxylate (600 mg, 1.898 mmol, 1 equiv), trimethyl-1,3,5,2,4,6-trioxatriborinane (476.4 mg, 3.796 mmol, 2 equiv), Pd(dppf)Cl₂ (138.9 mg, 0.190 mmol, 0.1 equiv) and K₂CO₃ (786.8 mg, 5.694 mmol, 3 equiv) in DMF (9 mL) was stirred for overnight at 100° C. under nitrogen atmosphere. The reaction was diluted with water and extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 4-fluoro-6-methylisoindoline-2-carboxylate (350 mg, 73.39%) as a brown solid. LC-MS (ES, m/z)=237.10 [M-t-Bu+ACN+1]⁺.

Step 4. Preparation of 4-fluoro-6-methylisoindoline. A mixture of tert-butyl 4-fluoro-6-methylisoindoline-2-carboxylate (350 mg, 1.393 mmol, 1 equiv) in DCM (5 mL) was added TFA (1 mL) dropwise and stirred for 2 h at room temperature under the air atmosphere. The resulting mixture was concentrated under vacuum to afford 4-fluoro-6-methylisoindoline (500 mg, crude) as a brown oil. The crude product was used in the next step directly without further purification. LC-MS (ES, m/z)=152.15 [M+1]⁺.

Step 5. Preparation of (2-(2,6-bis(benzyloxy)pyridin-3-yl)benzo[d]oxazol-6-yl)(4-fluoro-6-methylisoindolin-2-yl)methanone. A mixture of 4-fluoro-6-methylisoindoline (250 mg, 1.654 mmol, 1 equiv), 2-[2,6-bis(benzyloxy)pyridin-3-yl]-1,3-benzoxazole-6-carboxylic acid (823.0 mg, 1.819 mmol, 1.1 equiv), NMI (543.09 mg, 6.616 mmol, 4 equiv) and TCFH (927.9 mg, 3.308 mmol, 2 equiv) in ACN (5.0 mL) was stirred for 2 h at room temperature under air atmosphere. The reaction was diluted with water and extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(4-fluoro-6-methylisoindolin-2-yl)methanone (60 mg, 6.20%) as a yellow solid. LC-MS (ES, m/z)=586.3 [M+1]⁺.

Step 6. Preparation of 3-(6-(4-fluoro-6-methylisoindoline-2-carbonyl)benzo[d]oxazol-2-yl)piperidine-2,6-dione. A mixture of (2-(2,6-bis(benzyloxy)pyridin-3-yl)benzo[d]oxazol-6-yl)(4-fluoro-6-methylisoindolin-2-yl)methanone (50 mg, 0.085 mmol, 1 equiv) and Pd/C (50 mg, Palladium 10% on Carbon (wetted with ca. 55% Water) in DMF (1.0 mL) was stirred for overnight at room temperature under hydrogen atmosphere. The resulting mixture was filtered, the filter cake was washed with DMF (0.9 mL). The filtrate was concentrated under reduced pressure. The crude product (50 mg) was purified by Prep-HPLC to afford 3-(6-(4-fluoro-6-methylisoindoline-2-carbonyl)benzo[d]oxazol-2-yl)piperidine-2,6-dione (6.0 mg, 17%) as an off-white solid. LC-MS (ES, m/z)=408.20 [M+1]⁺. ¹H NMR (400 MHz, DMSO-d₆) δ 11.16 (s, 1H), 8.06 (dd, J=3.2, 1.5 Hz, 1H), 7.85 (dd, J=8.2, 1.4 Hz, 1H), 7.64 (ddd, J=8.2, 4.7, 1.5 Hz, 1H), 7.12-6.85 (m, 2H), 4.98-4.78 (m, 4H), 4.57 (ddd, J=11.2, 5.0, 1.2 Hz, 1H), 2.87-2.59 (m, 2H), 2.48-2.43 (m, 1H), 2.40-2.25 (m, 4H).

### Example 200

### Synthesis of 2-(2-(2,6-dioxopiperidin-3-yl)benzo[d]oxazole-6-carbonyl)-7-fluoroisoindoline-5-carbonitrile

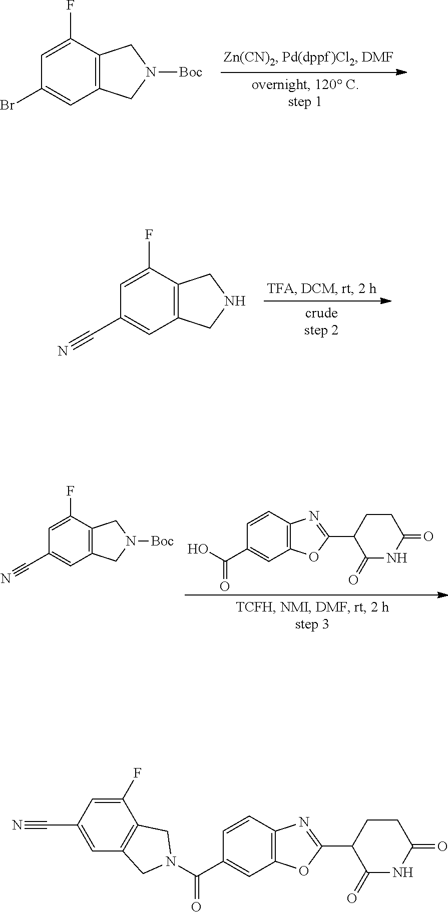 [SMILES: CC(C)(C)OC(=O)N1Cc2cc(Br)cc(F)c2C1 ; N#Cc1cc(F)c2c(c1)CNC2 ; CC(C)(C)OC(=O)N1Cc2cc(C#N)cc(F)c2C1 ; O=C1CCC(c2nc3ccc(C(=O)O)cc3o2)C(=O)N1 ; N#Cc1cc(F)c2c(c1)CN(C(=O)c1ccc3nc(C4CCC(=O)NC4=O)oc3c1)C2]

Step 1. Preparation of tert-butyl 6-cyano-4-fluoroisoindoline-2-carboxylate. A mixture of tert-butyl 6-bromo-4-fluoro-1,3-dihydroisoindole-2-carboxylate (700 mg, 2.214 mmol, 1 equiv), Zn(CN)₂ (909.9 mg, 7.749 mmol, 3.5 equiv) and Pd(dppf)Cl₂ (162.0 mg, 0.221 mmol, 0.1 equiv) in DMF (10 mL) was stirred for overnight at 120° C. under nitrogen atmosphere. The reaction was diluted with water at 0° C. The resulting mixture was extracted with DCM (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 6-cyano-4-fluoroisoindoline-2-carboxylate (290 mg, 50%) as a yellow solid. LC-MS (ES, m/z)=248.10 [M-t-Bu+ACN+1]⁺.

Step 2. Preparation of 7-fluoroisoindoline-5-carbonitrile. A mixture of tert-butyl 6-cyano-4-fluoroisoindoline-2-carboxylate (190 mg, 0.601 mmol, 1 equiv) in DCM (5 mL) was added and TFA (1 mL) dropwise and stirred for 2 h at room temperature under the air atmosphere. The resulting mixture was concentrated under vacuum to afford 7-fluoroisoindoline-5-carbonitrile (400 mg, crude) as a brown oil. The crude product was used in the next step directly without further purification. LC-MS (ES, m/z)=163.15 [M+1]⁺.

Step 3. Preparation of 2-(2-(2,6-dioxopiperidin-3-yl)benzo[d]oxazole-6-carbonyl)-7-fluoroisoindoline-5-carbonitrile. A mixture of 7-7-fluoroisoindoline-5-carbonitrile (90 mg, 0.555 mmol, 1 equiv), 2-(2,6-dioxopiperidin-3-yl)-1,3-benzoxazole-6-carboxylic acid (182.6 mg, 0.666 mmol, 1.2 equiv), NMI (182.3 mg, 2.220 mmol, 4 equiv) and TCFH (311.4 mg, 1.110 mmol, 2 equiv) in DMF (2 mL) was stirred for 2 h at room temperature under the air atmosphere. The resulting mixture was diluted with water (30 mL). The resulting mixture was extracted with EtOAc (3×20 mL). The combined organic layers were washed with brine (3×50 mL), dried over anhydrous Na₂SO₄. After filtration, the filtrate was concentrated under reduced pressure. The residue was purified by reversed-phase flash chromatography to afford 2 (2-(2,6-dioxopiperidin-3-yl)benzo[d]oxazole-6-carbonyl)-7-fluoroisoindoline-5-carbonitrile (37 mg, 15%) as off-white solid. LC-MS (ES, m/z)=419.05 [M+1]⁺. ¹H NMR (400 MHz, DMSO-d₆) δ 11.17 (s, 1H), 8.07 (dd, J=7.9, 1.6 Hz, 1H), 7.88-7.81 (m, 2H), 7.67-7.58 (m, 2H), 5.02 (s, 1H), 4.99 (s, 2H), 4.91 (s, 1H), 4.61-4.54 (m, 1H), 2.82-2.58 (m, 2H), 2.50-2.40 (m, 1H), 2.40-2.29 (m, 1H).

### Example 201

### Synthesis of 3-(5-(7,7-difluoro-2-azaspiro[4.4]nonane-2-carbonyl)-1-oxoisoindolin-2-yl)piperidine-2,6-dione

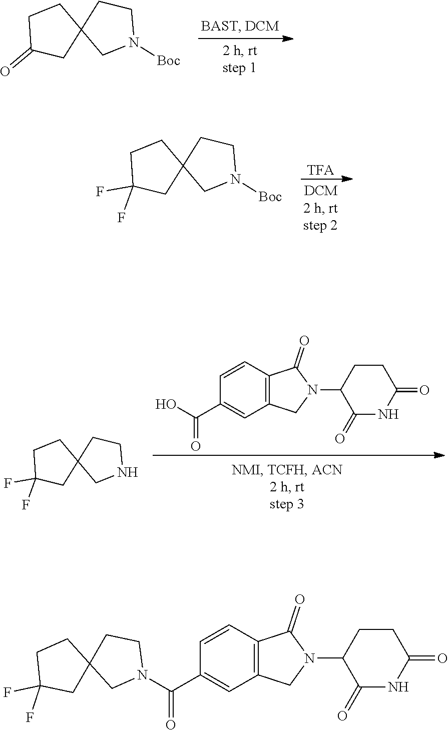 [SMILES: CC(C)(C)OC(=O)N1CCC2(CCC(=O)C2)C1 ; CC(C)(C)OC(=O)N1CCC2(CCC(F)(F)C2)C1 ; "TFA" ; FC1(F)CCC2(CCNC2)C1 ; O=C1CCC(N2Cc3cc(C(=O)O)ccc3C2=O)C(=O)N1 ; O=C1CCC(N2Cc3cc(C(=O)N4CCC5(CCC(F)(F)C5)C4)ccc3C2=O)C(=O)N1]

Step 1. Preparation of tert-butyl 7,7-difluoro-2-azaspiro[4.4]nonane-2-carboxylate. To a stirred mixture of tert-butyl 7-oxo-2-azaspiro[4.4]nonane-2-carboxylate (110 mg, 0.460 mmol, 1 equiv) in DCM (3 mL) was added BAST (508.4 mg, 2.300 mmol, 5 equiv) at 0° C. under nitrogen atmosphere. The resulting mixture was stirred for 2 h at room temperature. The reaction was quenched by the addition of sat. NH₄Cl (aq.) (2 mL) at 0° C. The resulting mixture was extracted with DCM (3×50 mL). The combined organic layers were washed with brine (3×50 mL), dried over anhydrous Na₂SO₄. After filtration, the filtrate was concentrated under reduced pressure. The residue was purified by reversed-phase flash chromatography to afford tert-butyl 7,7-difluoro-2-azaspiro[4.4]nonane-2-carboxylate (90 mg, 75%) as white solid. LC-MS (ES, m/z)=247.15 [M-t-Bu+ACN+1]⁺.

Step 2. Preparation of 7,7-difluoro-2-azaspiro[4.4]nonane. To a stirred mixture of tert-butyl 7,7-difluoro-2-azaspiro[4.4]nonane-2-carboxylate (60 mg, 0.230 mmol, 1 equiv) in DCM (1 mL) was added TFA (0.2 mL) dropwise at 0° C. under nitrogen atmosphere. The resulting mixture was stirred for 2 h at room temperature under nitrogen atmosphere. The resulting mixture was concentrated under reduced pressure to afford 7,7-difluoro-2-azaspiro[4.4]nonane (30 mg, 81%) as yellow oil. The crude product was used in the next step directly without further purification. LC-MS (ES, m/z)=162.15 [M+1]⁺.

Step 3. Preparation of 3-(5-(7,7-difluoro-2-azaspiro[4.4]nonane-2-carbonyl)-1-oxoisoindolin-2-yl)piperidine-2,6-dione. To a stirred mixture of 7,7-difluoro-2-azaspiro[4.4]nonane (30 mg, 0.186 mmol, 1 equiv) and 2-(2,6-dioxopiperidin-3-yl)-1-oxo-3H-isoindole-5-carboxylic acid (53.6 mg, 0.186 mmol, 1 equiv) in ACN (1 mL) were added TCFH (78.3 mg, 0.279 mmol, 1.5 equiv) and NMI (61.1 mg, 0.744 mmol, 4 equiv) at room temperature. The resulting mixture was stirred for 2 h at room temperature. The resulting mixture was diluted with H₂O (30 mL). The resulting mixture was extracted with EtOAc (3×20 mL). The combined organic layers were washed with brine (3×30 mL), dried over anhydrous Na₂SO₄. After filtration, the filtrate was concentrated under reduced pressure. The residue was purified by reversed-phase flash chromatography to afford 3-(5-(7,7-difluoro-2-azaspiro[4.4]nonane-2-carbonyl)-1-oxoisoindolin-2-yl)piperidine-2,6-dione (3.1 mg, 3.86%) as white solid. LC-MS (ES, m/z)=432.15 [M+1]⁺. ¹H NMR (400 MHz, DMSO-d₆) δ 11.00 (s, 1H), 7.81-7.70 (m, 2H), 7.63 (dd, J=16.7, 7.9 Hz, 1H), 5.14 (dd, J=13.3, 5.1 Hz, 1H), 4.50 (d, J=17.3 Hz, 1H), 4.37 (d, J=17.6 Hz, 1H), 3.56 (t, J=7.1 Hz, 1H), 3.49-3.34 (m, 2H), 2.96-2.87 (m, 1H), 2.61 (d, J=17.0 Hz, 1H), 2.42 (d, J=13.1 Hz, 1H), 2.34-2.00 (m, 6H), 1.97-1.80 (m, 3H), 1.78-1.70 (m, 1H).

### Example 202

### Synthesis of 3-(5-(3-fluoro-3-(2-fluorophenyl)azetidine-1-carbonyl)-1-oxoisoindolin-2-yl)piperidine-2,6-dione

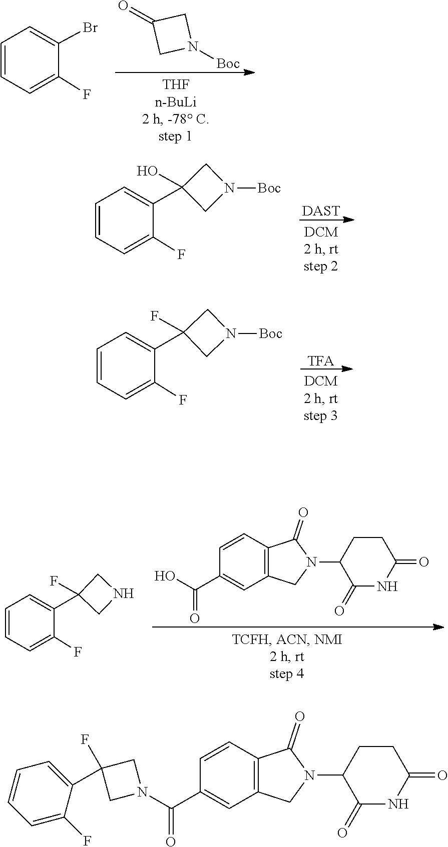

Step 1. Preparation of tert-butyl 3-(2-fluorophenyl)-3-hydroxyazetidine-1-carboxylate. To a stirred solution of 1-bromo-2-fluorobenzene (1.0 g, 5.841 mmol, 1 equiv) in THF (10 mL) was added n-BuLi in hexanes (3.5 mL, 8.761 mmol, 1.5 equiv) dropwise at −78° C.; under nitrogen atmosphere and stirred for 30 min under −78° C. To the above mixture was added tert-butyl 3-oxoazetidine-1-carboxylate (1 g, 5.841 mmol, 1 equiv) in THF dropwise and stirred for 2 h at −78° C. under nitrogen atmosphere. The reaction was quenched with sat. NH₄Cl (aq.) at 0° C. and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 3-(2-fluorophenyl)-3-hydroxyazetidine-1-carboxylate (1 g, 64%) as yellow solid. LC-MS (ES, m/z)=209.15 [M-t-Bu+1]⁺.

Step 2. Preparation of tert-butyl 3-fluoro-3-(2-fluorophenyl)azetidine-1-carboxylate. To a stirred mixture of tert-butyl 3-(2-fluorophenyl)-3-hydroxyazetidine-1-carboxylate (500 mg, 1.871 mmol, 1 equiv) in DCM (10 mL) was added DAST (603.0 mg, 3.742 mmol, 2 equiv) at 0° C. under nitrogen atmosphere. The resulting mixture was stirred for 2 h at room temperature under nitrogen atmosphere. The reaction was quenched with sat. NH₄Cl (aq.) at 0° C. and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 3-fluoro-3-(2-fluorophenyl)azetidine-1-carboxylate (200 mg, 40%) as white solid. LC-MS (ES, m/z)=255.20 [M-t-Bu+ACN+1]⁺.

Step 3. Preparation of 3-fluoro-3-(2-fluorophenyl)azetidine. To a stirred mixture of tert-butyl 3-fluoro-3-(2-fluorophenyl)azetidine-1-carboxylate (300 mg, 1.114 mmol, 1 equiv) in DCM (6 mL) was added TFA (1.2 mL) dropwise at 0° C. under nitrogen atmosphere. The resulting mixture was stirred for 2 h at room temperature under nitrogen atmosphere. The resulting mixture was concentrated under reduced pressure. The residue was purified by reversed-phase flash chromatography to afford 3-fluoro-3-(2-fluorophenyl)azetidine (180 mg, 96%) as white oil. LC-MS (ES, m/z)=170.10 [M+1]⁺.

Step 4. Preparation of 3-(5-(3-fluoro-3-(2-fluorophenyl)azetidine-1-carbonyl)-1-oxoisoindolin-2-yl)piperidine-2,6-dione. To a stirred mixture of 3-fluoro-3-(2-fluorophenyl)azetidine (117.3 mg, 0.694 mmol, 2 equiv) and 2-(2,6-dioxopiperidin-3-yl)-1-oxo-3H-isoindole-5-carboxylic acid (100 mg, 0.347 mmol, 1.00 equiv) in ACN (2 mL) were added TCFH (146.0 mg, 0.520 mmol, 1.5 equiv) and NMI (113.9 mg, 1.388 mmol, 4 equiv) at room temperature. The resulting mixture was stirred for 2 h at room temperature. The resulting mixture was diluted with H₂O (30 mL) and extracted with EtOAc (3×20 mL). The combined organic layers were washed with brine (3×50 mL), dried over anhydrous Na₂SO₄. After filtration, the filtrate was concentrated under reduced pressure. The crude product (80 mg) was purified by Prep-HPLC to afford 3-(5-(3-fluoro-3-(2-fluorophenyl)azetidine-1-carbonyl)-1-oxoisoindolin-2-yl)piperidine-2,6-dione (22 mg, 14%) as white solid. LC-MS (ES, m/z)=440.10 [M+1]⁺. ¹H NMR (400 MHz, DMSO-d₆) δ 11.01 (s, 1H), 7.95 (s, 1H), 7.81 (t, J=1.2 Hz, 2H), 7.62 (tt, J=7.7, 1.7 Hz, 1H), 7.59-7.49 (m, 1H), 7.38-7.26 (m, 2H), 5.15 (dd, J=13.3, 5.1 Hz, 1H), 5.03-4.71 (m, 3H), 4.57 (d, J=21.4 Hz, 1H), 4.50 (s, 1H), 4.40 (d, J=17.6 Hz, 1H), 2.92 (ddd, J=17.2, 13.6, 5.4 Hz, 1H), 2.66-2.56 (m, 1H), 2.40 (td, J=13.2, 4.5 Hz, 1H), 2.08-1.99 (m, 1H).

### Example 203

### Synthesis of 3-(5-(6-fluoro-6-(3-fluorophenyl)-2-azaspiro[3.3]heptane-2-carbonyl)-1-oxoisoindolin-2-yl)piperidine-2,6-dione

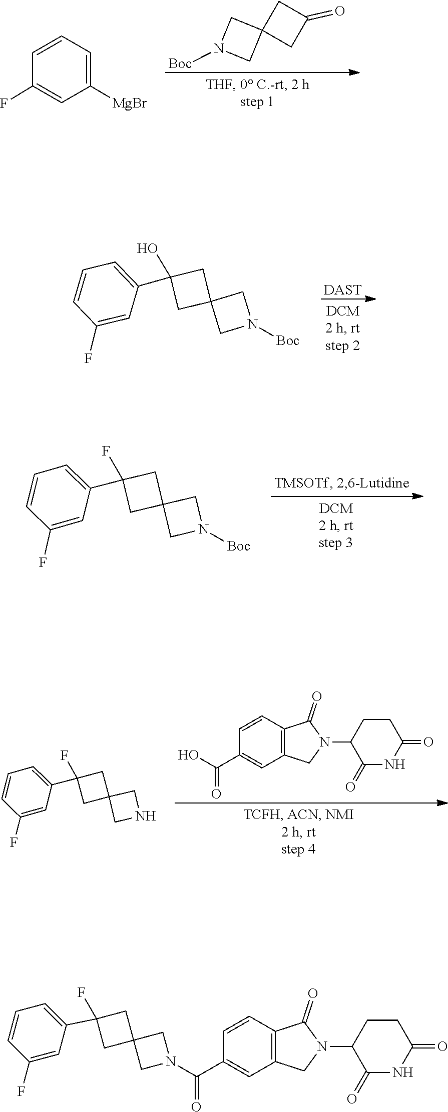

Step 1. Preparation of tert-butyl 6-(3-fluorophenyl)-6-hydroxy-2-azaspiro[3.3]heptane-2-carboxylate. To a stirred mixture of tert-butyl 6-oxo-2-azaspiro[3.3]heptane-2-carboxylate (1 g, 4.733 mmol, 1 equiv) in THF (10 mL) were added bromo(3-fluorophenyl)magnesium (7.1 mL, 1.0 M, 1.5 equiv) at 0° C. under nitrogen atmosphere. The resulting mixture was stirred for 2 h at room temperature under nitrogen atmosphere. The reaction was quenched by the addition of sat. NH₄Cl (aq.) at 0° C. and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6-(3-fluorophenyl)-6-hydroxy-2-azaspiro[3.3]heptane-2-carboxylate (800 mg, 55%) as yellow solid. LC-MS (ES, m/z)=308.20 [M+1]⁺.

Step 2. Preparation of tert-butyl 6-fluoro-6-(3-fluorophenyl)-2-azaspiro[3.3]heptane-2-carboxylate. To a stirred mixture of tert-butyl 6-(3-fluorophenyl)-6-hydroxy-2-azaspiro[3.3]heptane-2-carboxylate (714 mg, 2.323 mmol, 1 equiv) in DCM (15 mL) was added DAST (748.8 mg, 4.646 mmol, 2 equiv) at 0° C. under nitrogen atmosphere. The resulting mixture was stirred for 2 h at room temperature under nitrogen atmosphere. The reaction was quenched with sat. NH₄Cl (aq.) at 0° C. and extracted with EtOAc (3×50 mL). The combined organic layers were washed with brine (2×30 mL), dried over anhydrous Na₂SO₄. After filtration, the filtrate was concentrated under reduced pressure. The residue was purified by reversed-phase flash chromatography to afford tert-butyl 6-fluoro-6-(3-fluorophenyl)-2-azaspiro[3.3]heptane-2-carboxylate (450 mg, 63%) as white solid. LC-MS (ES, m/z)=295.15 [M-t-Bu+ACN+1]⁺.

Step 3. Preparation of 6-fluoro-6-(3-fluorophenyl)-2-azaspiro[3.3]heptane. To a stirred mixture of tert-butyl 6-fluoro-6-(3-fluorophenyl)-2-azaspiro[3.3]heptane-2-carboxylate (250 mg, 0.808 mmol, 1 equiv) and 2,6-Lutidine (432.9 mg, 4.040 mmol, 5 equiv) in DCM (10 mL) was added TMSOTf (718.4 mg, 3.232 mmol, 4 equiv) at 0° C. under nitrogen atmosphere. The reaction was quenched with water (50 mL) at 0° C. and extracted with EtOAc (3×50 mL). The combined organic layers were washed with brine (2×100 mL), dried over anhydrous Na₂SO₄. After filtration, the filtrate was concentrated under reduced pressure. The resulting mixture was concentrated under reduced pressure. The residue was purified by reversed-phase flash chromatography to afford 6-fluoro-6-(3-fluorophenyl)-2-azaspiro[3.3]heptane (150 mg, 89%) as white oil. LC-MS (ES, m/z)=210.20 [M+1]⁺.

Step 4. Preparation of 3-(5-(6-fluoro-6-(3-fluorophenyl)-2-azaspiro[3.3]heptane-2-carbonyl)-1-oxoisoindolin-2-yl)piperidine-2,6-dione. To a stirred mixture of 6-fluoro-6-(3-fluorophenyl)-2-azaspiro[3.3]heptane (72.5 mg, 0.347 mmol, 1 equiv) and 2-(2,6-dioxopiperidin-3-yl)-1-oxo-3H-isoindole-5-carboxylic acid (100 mg, 0.347 mmol, 1.00 equiv) in ACN (2 mL) were added TCFH (146 mg, 0.520 mmol, 1.5 equiv) and NMI (113.9 mg, 1.388 mmol, 4 equiv) at room temperature. The resulting mixture was stirred for 2 h at room temperature. The reaction was diluted with water (30 mL) at 0° C. and extracted with EtOAc (3×20 mL). The combined organic layers were washed with brine (2×50 mL), dried over anhydrous Na₂SO₄. After filtration, the filtrate was concentrated under reduced pressure. The resulting mixture was concentrated under reduced pressure. The crude product (70 mg) was purified by Prep-HPLC to afford 3-(5-(6-fluoro-6-(3-fluorophenyl)-2-azaspiro[3.3]heptane-2-carbonyl)-1-oxoisoindolin-2-yl)piperidine-2,6-dione (18 mg, 11%) as a white solid. LC-MS (ES, m/z)=480.15 [M+1]⁺. ¹H NMR (400 MHz, DMSO-d₆) δ 11.01 (s, 1H), 7.91-7.69 (m, 3H), 7.48 (d, J=10.1 Hz, 1H), 7.34-7.09 (m, 3H), 5.14 (d, J=13.2 Hz, 1H), 4.51 (q, J=9.6 Hz, 2H), 4.43-4.31 (m, 2H), 4.25 (s, 1H), 4.10 (s, 1H), 3.09-2.67 (m, 5H), 2.61 (d, J=17.3 Hz, 1H), 2.41 (d, J=13.5 Hz, 1H), 2.03 (s, 1H).

### Example 204

### Synthesis of 3-(5-(6-fluoro-6-(2-fluorophenyl)-2-azaspiro[3.3]heptane-2-carbonyl oxoisoindolin-2-yl)piperidine-2,6-dione

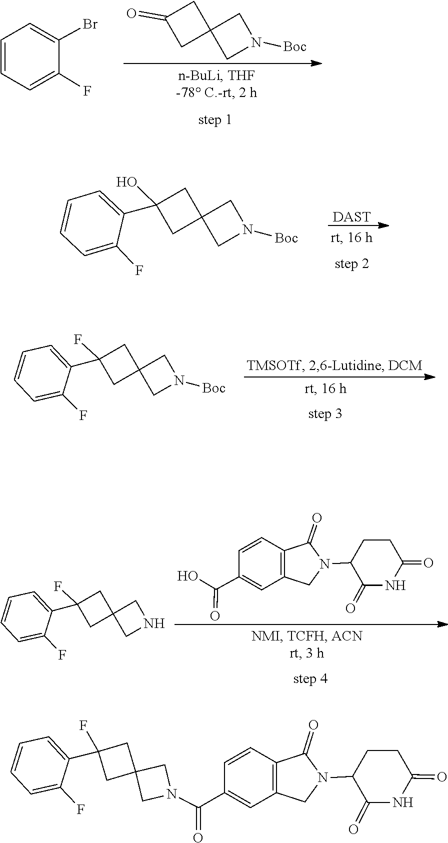

Step 1. Preparation of tert-butyl 6-(2-fluorophenyl)-6-hydroxy-2-azaspiro[3.3]heptane-2-carboxylate. To a stirred solution of 1-bromo-2-fluorobenzene (2.5 g, 14.286 mmol, 1.51 equiv) in THF (30 mL) were added n-BuLi (7.6 mL, 19.000 mmol, 2.01 equiv) dropwise at −78° C. under nitrogen atmosphere. The resulting mixture was stirred for 30 min at −78° C. under nitrogen atmosphere. To the above mixture was added tert-butyl 6-oxo-2-azaspiro[3.3]heptane-2-carboxylate (2 g, 9.467 mmol, 1 equiv) in THF (10 mL) dropwise over 20 min at −78° C. The resulting mixture was stirred for an additional 2 h at room temperature. The reaction was quenched with sat. NH₄Cl (aq.) at 0° C. The resulting mixture was diluted with water (200 mL) and extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford tert-butyl 6-(2-fluorophenyl)-6-hydroxy-2-azaspiro[3.3]heptane-2-carboxylate (1.5 g, 52%) as off-white solid. LC-MS (ES, m/z)=308.15 [M+1]⁺.

Step 2. Preparation of tert-butyl 6-fluoro-6-(2-fluorophenyl)-2-azaspiro[3.3]heptane-2-carboxylate. To a stirred mixture of tert-butyl 6-(2-fluorophenyl)-6-hydroxy-2-azaspiro[3.3]heptane-2-carboxylate (1.1 g, 3.579 mmol, 1 equiv) in DCM (20 mL) was added DAST (1.1 g, 7.158 mmol, 2 equiv) in portions at 0° C. under nitrogen atmosphere. The resulting mixture was stirred for 16 h at room temperature under nitrogen atmosphere. The resulting mixture was diluted with water (150 mL). The resulting mixture was diluted with DCM (3×200 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (5:1) to afford tert-butyl 6-fluoro-6-(2-fluorophenyl)-2-azaspiro[3.3]heptane-2-carboxylate (1 g, 90%) as colorless oil. LC-MS (ES, m/z)=310.20 [M+1]⁺.

Step 3. Preparation of 6-fluoro-6-(2-fluorophenyl)-2-azaspiro[3.3]heptane. To a stirred solution of tert-butyl 6-fluoro-6-(2-fluorophenyl)-2-azaspiro[3.3]heptane-2-carboxylate (0.9 g, 2.909 mmol, 1 equiv) and 2,6-Lutidine (1.5 g, 14.545 mmol, 5 equiv) in DCM (30 mL) was added TMSOTf (2.5 g, 11.636 mmol, 4 equiv) dropwise at 0° C. The resulting mixture was stirred for 16 h at room temperature. The resulting mixture was diluted with water (100 mL). The resulting mixture was diluted with DCM (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EA (5:1) to afford 6-fluoro-6-(2-fluorophenyl)-2-azaspiro[3.3]heptane (500 mg, 82%) as brown oil. LC-MS (ES, m/z)=210.0 [M+1]⁺.

Step 4. Preparation of 6-fluoro-6-(2-fluorophenyl)-2-azaspiro[3.3]hep 3-(5-(6-fluoro-6-(2-fluorophenyl)-2-azaspiro[3.3]heptane-2-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (100 mg, 0.347 mmol, 1 equiv) and NMI (85.4 mg, 1.041 mmol, 3 equiv) in ACN (2.0 mL) were added 6-fluoro-6-(2-fluorophenyl)-2-azaspiro[3.3]heptane (108.8 mg, 0.520 mmol, 1.5 equiv) and TCFH (146.0 mg, 0.520 mmol, 1.5 equiv) in portions at room temperature. The resulting mixture was stirred for 3 h at room temperature. The resulting mixture was diluted with water (50 mL). The resulting mixture was extracted with EtOAc (3×20 mL). The combined organic layers were washed with brine (3×30 mL), dried over anhydrous Na₂SO₄. After filtration, the filtrate was concentrated under reduced pressure. The crude product (86 mg) was purified by Prep-HPLC to afford 3-(5-(6-fluoro-6-(2-fluorophenyl)-2-azaspiro[3.3]heptane-2-carbonyl)-1-oxoisoindolin-2-yl)piperidine-2,6-dione (30 mg, 18%) as white solid. LC-MS (ES, m/z)=480.20 [M+1]⁺. ¹H NMR (400 MHz, DMSO-d₆) δ 11.00 (s, 1H), 7.88-7.67 (m, 3H), 7.45-7.34 (m, 2H), 7.19 (dt, J=13.0, 8.9 Hz, 2H), 5.19-5.09 (m, 1H), 4.43 (h, J=16.8 Hz, 5H), 4.17 (d, J=12.5 Hz, 1H), 3.06-2.84 (m, 5H), 2.81-2.58 (m, 1H), 2.51-2.49 (m, 1H), 2.40-2.00 (m, 1H).

### Example 205

### Synthesis of 3-(5-(6-(4-fluorophenyl)-2-azaspiro[3.3]heptane-2-carbonyl)-1-oxoisoindolin-2-yl)piperidine-2,6-dione

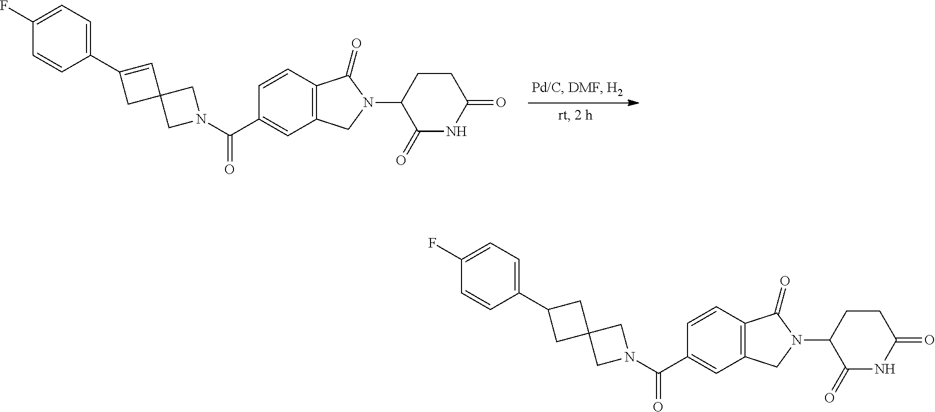 [SMILES: O=C1CCC(N2Cc3cc(C(=O)N4CC5(C=C(c6ccc(F)cc6)C5)C4)ccc3C2=O)C(=O)N1 ; O=C1CCC(N2Cc3cc(C(=O)N4CC5(CC(c6ccc(F)cc6)C5)C4)ccc3C2=O)C(=O)N1]

Step 1. Preparation of 3-(5-(6-(4-fluorophenyl)-2-azaspiro[3.3]heptane-2-carbonyl)-1-oxoisoindolin-2-yl)piperidine-2,6-dione. To a solution of 3-{5-[6-(4-fluorophenyl)-2-azaspiro[3.3]hept-5-ene-2-carbonyl]-1-oxo-3H-isoindol-2-yl}piperidine-2,6-dione (60 mg, 0.131 mmol, 1 equiv) in dimethylformamide (1 mL) was added Pd/C (30 mg, Palladium 10% on Carbon (wetted with ca. 55% Water)) under nitrogen atmosphere in a 8 mL vial. The mixture was hydrogenated at room temperature for 2 h under hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford 3-(5-(6-(4-fluorophenyl)-2-azaspiro[3.3]heptane-2-carbonyl)-1-oxoisoindolin-2-yl)piperidine-2,6-dione (24 mg, 39%) as an off-white solid. LC-MS (ES, m/z)=462.25 [M+1]⁺. ¹H NMR (400 MHz, DMSO-d₆) δ 11.01 (s, 1H), 7.87 (d, J=10.4 Hz, 1H), 7.83-7.70 (m, 2H), 7.25 (ddd, J=8.6, 5.5, 2.7 Hz, 2H), 7.11 (td, J=8.9, 2.1 Hz, 2H), 5.14 (dd, J=12.6, 4.4 Hz, 1H), 4.55-4.34 (m, 3H), 4.24 (d, J=4.3 Hz, 2H), 4.01 (s, 1H), 3.46-3.34 (m, 1H), 2.99-2.85 (m, 1H), 2.61 (d, J=17.4 Hz, 3H), 2.42 (d, J=13.3 Hz, 1H), 2.26 (q, J=12.2 Hz, 2H), 2.07-1.99 (m, 1H).

### Example 206

### Synthesis of 3-(6-(6-(4-fluorophenyl)-2-azaspiro[3.3]hept-5-ene-2-carbonyl benzo[d]oxazol-2-yl)piperidine-2,6-dione

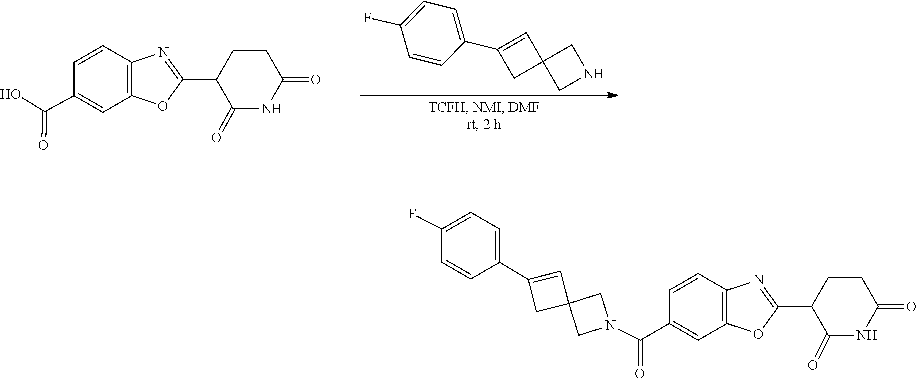 [SMILES: O=C1CCC(c2nc3ccc(C(=O)O)cc3o2)C(=O)N1 ; Fc1ccc(C2=CC3(CNC3)C2)cc1 ; O=C1CCC(c2nc3ccc(C(=O)N4CC5(C=C(c6ccc(F)cc6)C5)C4)cc3o2)C(=O)N1]

Step 1. Preparation of 3-(6-(6-(4-fluorophenyl)-2-azaspiro[3.3]hept-5-ene-2-carbonyl)benzo[d]oxazol-2-yl)piperidine-2,6-dione. To a stirred mixture of 6-(4-fluorophenyl)-2-azaspiro[3.3]hept-5-ene (103.5 mg, 0.547 mmol, 1.50 equiv) and 2-(2,6-dioxopiperidin-3-yl)-1,3-benzoxazole-6-carboxylic acid (100 mg, 0.365 mmol, 1.00 equiv) in DMF (2 mL) were added NMI (149.7 mg, 1.823 mmol, 5.00 equiv) and TCFH (153.5 mg, 0.547 mmol, 1.50 equiv) at room temperature under air atmosphere. The resulting mixture was stirred for 2 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(6-(6-(4-fluorophenyl)-2-azaspiro[3.3]hept-5-ene-2-carbonyl)benzo[d]oxazol-2-yl)piperidine-2,6-dione (12 mg, 7.26%) as an off-white solid. LC-MS (ES, m/z)=446.20 [M+1]⁺. ¹H NMR (400 MHz, DMSO-d₆) δ 11.15 (s, 1H), 8.03 (d, J=1.4 Hz, 1H), 7.85-7.68 (m, 2H), 7.50-7.40 (m, 2H), 7.26-7.15 (m, 2H), 6.51 (s, 1H), 4.56 (dq, J=12.4, 7.6, 5.9 Hz, 3H), 4.37-4.22 (m, 2H), 2.98 (d, J=2.0 Hz, 2H), 2.78-2.60 (m, 2H), 2.47-2.29 (m, 2H), 1.36 (s, 1H).

### Example 207

### Synthesis of 3-(5-(6-(3-fluorophenyl)-2-azaspiro[3.3]hept-5-ene-2-carbonyl)-1-oxoisoindolin-2-yl)piperidine-2,6-dione

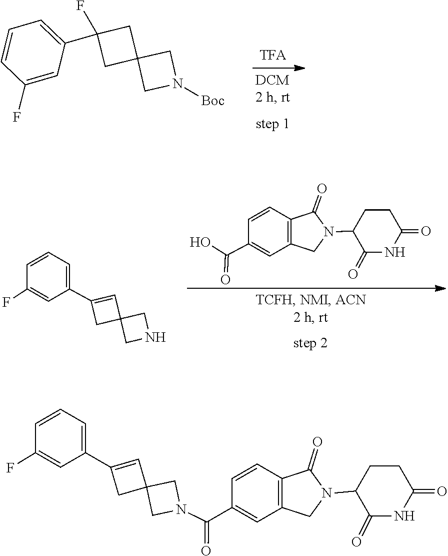 [SMILES: CC(C)(C)OC(=O)N1CC2(C1)CC(F)(c1cccc(F)c1)C2 ; Fc1cccc(C2=CC3(CNC3)C2)c1 ; O=C1CCC(N2Cc3cc(C(=O)O)ccc3C2=O)C(=O)N1 ; O=C1CCC(N2Cc3cc(C(=O)N4CC5(C=C(c6cccc(F)c6)C5)C4)ccc3C2=O)C(=O)N1]

Step 1. Preparation of 6-(3-fluorophenyl)-2-azaspiro[3.3]hept-5-ene. To a stirred mixture of tert-butyl 6-fluoro-6-(3-fluorophenyl)-2-azaspiro[3.3]heptane-2-carboxylate (150 mg, 0.485 mmol, 1 equiv) in DCM (1.5 mL) was added TFA (0.3 mL) at 0° C. under nitrogen atmosphere. The resulting mixture was stirred for 2 h at room temperature. The resulting mixture was concentrated under reduced pressure. The residue was purified by reversed-phase flash chromatography to afford 6-(3-fluorophenyl)-2-azaspiro[3.3]hept-5-ene (80 mg, 87%) as a yellow oil. LC-MS (ES, m/z)=190.20 [M+1]⁺.

Step 2. Preparation of 3-(5-(6-(3-fluorophenyl)-2-azaspiro[3.3]hept-5-ene-2-carbonyl)-1-oxoisoindolin-2-yl)piperidine-2,6-dione. To a stirred mixture of 6-(3-fluorophenyl)-2-azaspiro[3.3]hept-5-ene (49.2 mg, 0.259 mmol, 1.50 equiv) and 2-(2,6-dioxopiperidin-3-yl)-1-oxo-3H-isoindole-5-carboxylic acid (50 mg, 0.173 mmol, 1.00 equiv) in ACN (2 mL) were added TCFH (73.0 mg, 0.259 mmol, 1.5 equiv) and NMI (56.9 mg, 0.692 mmol, 4 equiv) at room temperature. The resulting mixture was stirred for 2 h at room temperature. The resulting mixture was diluted with water (30 mL). The resulting mixture was extracted with EtOAc (3×20 mL). The combined organic layers were washed with brine (3×50 mL), dried over anhydrous Na₂SO₄. After filtration, the filtrate was concentrated under reduced pressure. The crude product (50 mg) was purified by Prep-HPLC to afford 3-(5-(6-(3-fluorophenyl)-2-azaspiro[3.3]hept-5-ene-2-carbonyl)-1-oxoisoindolin-2-yl)piperidine-2,6-dione (9 mg, 11%) as a white solid. LC-MS (ES, m/z)=460.15 [M+1]⁺. ¹H NMR (400 MHz, DMSO-d₆) δ 11.01 (s, 1H), 7.90 (s, 1H), 7.79 (s, 2H), 7.42 (q, J=7.6 Hz, 1H), 7.23 (d, J=6.8 Hz, 2H), 7.15 (t, J=8.5 Hz, 1H), 6.64 (s, 1H), 5.14 (dd, J=13.2, 5.2 Hz, 1H), 4.58-4.48 (m, 3H), 4.42-4.29 (m, 3H), 3.02-2.89 (m, 3H), 2.63 (s, 1H), 2.41 (s, 1H), 2.03 (s, 1H), 1.23 (s, 1H).

### Example 208

### Synthesis of 3-(5-(6-(2-fluorophenyl)-2-azaspiro[3.3]hept-5-ene-2-carbonyl)-1-oxoisoindolin-2-yl)piperidine-2,6-dione

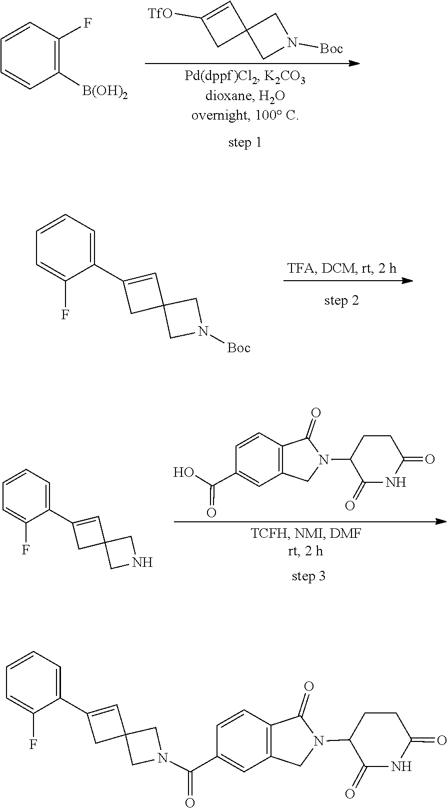 [SMILES: OB(O)c1ccccc1F ; CC(C)(C)OC(=O)N1CC2(C=C(OS(=O)(=O)C(F)(F)F)C2)C1 ; CC(C)(C)OC(=O)N1CC2(C=C(c3ccccc3F)C2)C1 ; Fc1ccccc1C1=CC2(CNC2)C1 ; O=C1CCC(N2Cc3cc(C(=O)O)ccc3C2=O)C(=O)N1 ; O=C1CCC(N2Cc3cc(C(=O)N4CC5(C=C(c6ccccc6F)C5)C4)ccc3C2=O)C(=O)N1]

Step 1. Preparation of tert-butyl 6-(2-fluorophenyl)-2-azaspiro[3.3]hept-5-ene-2-carboxylate. To a solution of tert-butyl 6-(trifluoromethanesulfonyloxy)-2-azaspiro[3.3]hept-5-ene-2-carboxylate (1 g, 2.913 mmol, 1 equiv) and 2-fluorophenylboronic acid (0.61 g, 4.360 mmol, 1.50 equiv) in dioxane (10 mL) and H₂O (1 mL) were added K₂CO₃ (1.21 g, 8.739 mmol, 3 equiv) and Pd(dppf)Cl₂ (0.21 g, 0.291 mmol, 0.1 equiv). After stirring overnight at 100° C. under a nitrogen atmosphere, the resulting mixture was diluted with water (100 mL) and extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford tert-butyl 6-(2-fluorophenyl)-2-azaspiro[3.3]hept-5-ene-2-carboxylate (300 mg, 36%) as a brown oil. LC-MS (ES, m/z)=275.15 [M-t-Bu+ACN+1]⁺.

Step 2. Preparation of 6-(2-fluorophenyl)-2-azaspiro[3.3]hept-5-ene. A mixture of tert-butyl 6-(2-fluorophenyl)-2-azaspiro[3.3]hept-5-ene-2-carboxylate (380 mg, 1.313 mmol, 1 equiv) in DCM (5 mL) was added TFA (1 mL) dropwise and stirred for 2 h at room temperature under air atmosphere. The resulting mixture was concentrated under vacuum to afford 6-(2-fluorophenyl)-2-azaspiro[3.3]hept-5-ene (300 mg, crude) as a brown oil. The crude product was used in the next step directly without further purification. LC-MS (ES, m/z)=190.15 [M+1]⁺.

Step 3. Preparation of 3-(5-(6-(2-fluorophenyl)-2-azaspiro[3.3]hept-5-ene-2-carbonyl)-1-oxoisoindolin-2-yl)piperidine-2,6-dione. A mixture of 6-(2-fluorophenyl)-2-azaspiro[3.3]hept-5-ene (328.23 mg, 1.735 mmol, 5 equiv), 2-(2,6-dioxopiperidin-3-yl)-1-oxo-3H-isoindole-5-carboxylic acid (100 mg, 0.347 mmol, 1.00 equiv), NMI (284.8 mg, 3.470 mmol, 10 equiv) and TCFH (146.0 mg, 0.520 mmol, 1.5 equiv) in DMF (1.5 mL) was stirred for 2 h at room temperature under air atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The crude product was purified by Prep-HPLC to afford 3-(5-(6-(2-fluorophenyl)-2-azaspiro[3.3]hept-5-ene-2-carbonyl)-1-oxoisoindolin-2-yl)piperidine-2,6-dione (7.0 mg, 4.18%) as off-white solid. LC-MS (ES, m/z)=460.20 [M+1]⁺. ¹H NMR (400 MHz, DMSO-d₆) δ 11.01 (s, 1H), 7.89 (s, 1H), 7.84-7.71 (m, 2H), 7.44-7.30 (m, 2H), 7.27-7.18 (m, 2H), 6.53 (d, J=3.1 Hz, 1H), 5.14 (dd, J=13.3, 5.1 Hz, 1H), 4.61-4.47 (m, 3H), 4.42-4.25 (m, 3H), 3.06 (d, J=3.0 Hz, 2H), 2.92 (ddd, J=17.2, 13.6, 5.4 Hz, 1H), 2.69-2.56 (m, 2H), 2.47-2.28 (m, 2H), 2.03 (dtd, J=12.7, 5.3, 2.3 Hz, 1H).

### Example 209

### Synthesis of 3-(1-oxo-5-(6-(4-(trifluoromethyl)phenyl)-2-azaspiro[3.3]hept-5-ene-2-carbonyl)isoindolin-2-yl)piperidine-2,6-dione

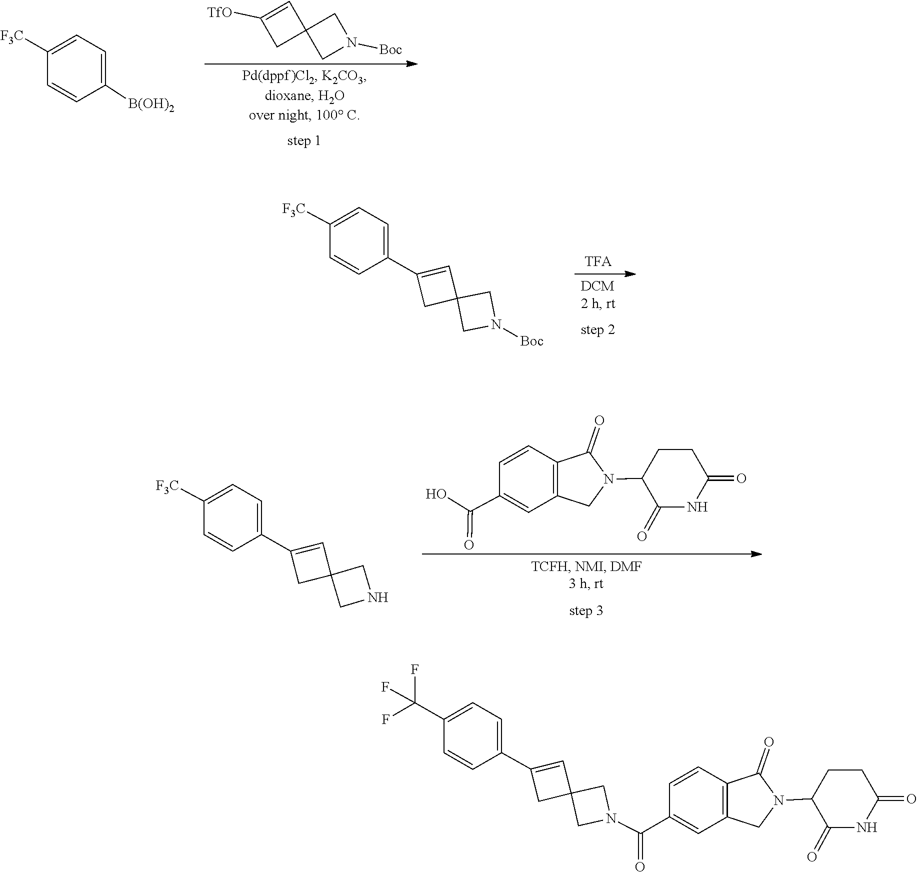 [SMILES: OB(O)c1ccc(C(F)(F)F)cc1 ; CC(C)(C)OC(=O)N1CC2(C=C(OS(=O)(=O)C(F)(F)F)C2)C1 ; CC(C)(C)OC(=O)N1CC2(C=C(c3ccc(C(F)(F)F)cc3)C2)C1 ; FC(F)(F)c1ccc(C2=CC3(CNC3)C2)cc1 ; O=C1CCC(N2Cc3cc(C(=O)O)ccc3C2=O)C(=O)N1 ; O=C1CCC(N2Cc3cc(C(=O)N4CC5(C=C(c6ccc(C(F)(F)F)cc6)C5)C4)ccc3C2=O)C(=O)N1]

Step 1. Preparation of tert-butyl 6-(4-(trifluoromethyl)phenyl)-2-azaspiro[3.3]hept-5-ene-2-carboxylate. To a stirred mixture of tert-butyl 6-(trifluoromethanesulfonyloxy)-2-azaspiro[3.3]hept-5-ene-2-carboxylate (1.5 g, 4.369 mmol, 1 equiv) and 4-(trifluoromethyl)phenylboronic acid (1.24 g, 6.529 mmol, 1.49 equiv) in dioxane (20 mL) and H₂O (2 mL) were added Pd(dppf)Cl₂ (319.7 mg, 0.437 mmol, 0.10 equiv) and K₂CO₃ (1.81 g, 13.107 mmol, 3 equiv) at room temperature under air atmosphere. The resulting mixture was stirred overnight at 100° C. under nitrogen atmosphere. The mixture was allowed to cool down to room temperature. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6-[4-(trifluoromethyl)phenyl]-2-azaspiro[3.3]hept-5-ene-2-carboxylate (1 g, 67%) as a brown solid. LC-MS (ES, m/z)=325.15 [M-t-Bu+ACN+1]⁺.

Step 2. Preparation of 6-[4-(trifluoromethyl)phenyl]-2-azaspiro[3.3]hept-5-ene. To a stirred solution of tert-butyl 6-[4-(trifluoromethyl)phenyl]-2-azaspiro[3.3]hept-5-ene-2-carboxylate (550 mg, 1.621 mmol, 1 equiv) in DCM (10 mL) was added TFA (2 mL, 26.926 mmol, 16.61 equiv) dropwise at 0° C. under nitrogen atmosphere. The resulting mixture was stirred for 2 h at room temperature under nitrogen atmosphere. The resulting mixture was concentrated under reduced pressure to afford in 6-[4-(trifluoromethyl)phenyl]-2-azaspiro[3.3]hept-5-ene (500 mg, crude) as a brown oil. The crude product was used in the next step directly without further purification. LC-MS (ES, m/z)=281.15 [M+ACN+1]⁺.

Step 3. Preparation of 3-(1-oxo-5-(6-(4-(trifluoromethyl)phenyl)-2-azaspiro[3.3]hept-5-ene-2-carbonyl)isoindolin-2-yl)piperidine-2,6-dione. To a stirred mixture of 6-[4-(trifluoromethyl)phenyl]-2-azaspiro[3.3]hept-5-ene (124.5 mg, 0.520 mmol, 1.00 equiv) and 2-(2,6-dioxopiperidin-3-yl)-1-oxo-3H-isoindole-5-carboxylic acid (150 mg, 0.520 mmol, 1.00 equiv) in DMF (3 mL) were added NMI (213.6 mg, 2.602 mmol, 5.00 equiv) and TCFH (219.0 mg, 0.781 mmol, 1.50 equiv) at room temperature under air atmosphere. The resulting mixture was stirred for an additional 3 h at room temperature. The resulting mixture was diluted with water (50 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford crude product (120 mg). The crude product was purified by Prep-HPLC to afford 3-(1-oxo-5-(6-(4-(trifluoromethyl)phenyl)-2-azaspiro[3.3]hept-5-ene-2-carbonyl)isoindolin-2-yl)piperidine-2,6-dione (68 mg, 26%) as an off-white solid. LC-MS (ES, m/z)=510.25 [M+1]⁺. ¹H NMR (400 MHz, DMSO-d₆) δ 11.01 (s, 1H), 7.90 (t, J=1.1 Hz, 1H), 7.84-7.76 (m, 2H), 7.74 (d, J=8.1 Hz, 2H), 7.61 (d, J=8.0 Hz, 2H), 6.76 (s, 1H), 5.14 (dd, J=13.3, 5.1 Hz, 1H), 4.61-4.47 (m, 3H), 4.43-4.23 (m, 3H), 3.04 (d, J=3.2 Hz, 2H), 2.92 (ddd, J=17.3, 13.6, 5.4 Hz, 1H), 2.66-2.56 (m, 1H), 2.42 (qd, J=13.2, 4.4 Hz, 2H), 2.03 (ddd, J=9.6, 5.4, 2.6 Hz, 1H).

### Example 210

### Synthesis of 3-(6-(4-fluoro-7-(trifluoromethoxy)isoindoline-2-carbonyl)benzo[d]oxazol-2-yl)piperidine-2,6-dione

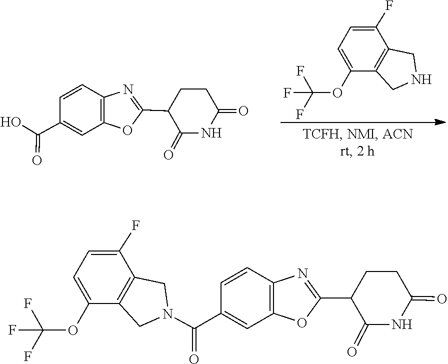 [SMILES: O=C1CCC(c2nc3ccc(C(=O)O)cc3o2)C(=O)N1 ; Fc1ccc(OC(F)(F)F)c2c1CNC2 ; O=C1CCC(c2nc3ccc(C(=O)N4Cc5c(F)ccc(OC(F)(F)F)c5C4)cc3o2)C(=O)N1]

Step 1. Preparation of 3-(6-(4-fluoro-7-(trifluoromethoxy)isoindoline-2-carbonyl)benzo[d]oxazol-2-yl)piperidine-2,6-dione. A mixture of 4-fluoro-7-(trifluoromethoxy)-2,3-dihydro-1H-isoindole (145.16 mg, 0.656 mmol, 1.2 equiv), 2-(2,6-dioxopiperidin-3-yl)-1,3-benzoxazole-6-carboxylic acid (150 mg, 0.547 mmol, 1.00 equiv), NMI (179.6 mg, 2.188 mmol, 4 equiv) and TCFH (306.9 mg, 1.094 mmol, 2 equiv) in ACN (2.5 mL) was stirred for 2 h at room temperature under air atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The crude product was purified by Prep-HPLC to afford 3-(6-(4-fluoro-7-(trifluoromethoxy)isoindoline-2-carbonyl)benzo[d]oxazol-2-yl)piperidine-2,6-dione (7.8 mg, 2.96%) as off-white solid. LC-MS (ES, m/z)=478.15 [M+1]⁺.

¹H NMR (400 MHz, DMSO-d₆) δ 11.16 (s, 1H), 8.07 (t, J=1.8 Hz, 1H), 7.86 (dd, J=8.2, 1.5 Hz, 1H), 7.66 (dd, J=8.2, 1.5 Hz, 1H), 7.50-7.25 (m, 2H), 5.09-4.88 (m, 4H), 4.57 (dd, J=11.3, 5.1 Hz, 1H), 2.71 (dddd, J=29.6, 17.4, 12.0, 5.0 Hz, 2H), 2.48-2.40 (m, 1H), 2.40-2.30 (m, 1H).

### Example 211

### Synthesis of 3-(6-(7′-fluorospiro[cyclopropane-1,1′-isoindoline]-2′-carbonyl)benzo[d]oxazol-2-yl)piperidine-2,6-dione

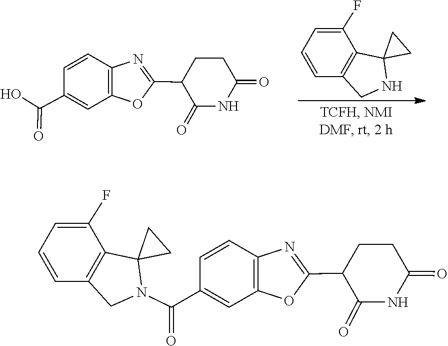 [SMILES: O=C1CCC(c2nc3ccc(C(=O)O)cc3o2)C(=O)N1 ; Fc1cccc2c1C1(CC1)NC2 ; O=C1CCC(c2nc3ccc(C(=O)N4Cc5cccc(F)c5C45CC5)cc3o2)C(=O)N1]

Step 1. Preparation of 3-(6-(7′-fluorospiro[cyclopropane-1,1′-isoindoline]-2′-carbonyl)benzo[d]oxazol-2-yl)piperidine-2,6-dione. To a stirred mixture of 2-(2,6-dioxopiperidin-3-yl)-1,3-benzoxazole-6-carboxylic acid (112.92 mg, 0.412 mmol, 1.2 equiv) and 7′-fluoro-2′,3′-dihydrospiro[cyclopropane-1,1′-isoindole] (56 mg, 0.343 mmol, 1.00 equiv) in dimethylformamide (2.0 mL) was added NMI (112.70 mg, 1.372 mmol, 4 equiv) and TCFH (192.56 mg, 0.686 mmol, 2 equiv) in portions at room temperature under nitrogen atmosphere. The resulting mixture was stirred for 2 h at room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The crude product (70 mg) was purified by Prep-HPLC to afford 3-(6-(7′-fluorospiro[cyclopropane-1,1′-isoindoline]-2′-carbonyl)benzo[d]oxazol-2-yl)piperidine-2,6-dione (29 mg, 20%) as off-white solid. LC-MS (ES, m/z)=478.15 [M+1]⁺. ¹H NMR (400 MHz, DMSO-d₆) δ 11.15 (s, 1H), 7.98 (d, J=1.4 Hz, 1H), 7.82 (d, J=8.1 Hz, 1H), 7.50 (dd, J=8.1, 1.5 Hz, 1H), 7.26 (td, J=7.9, 4.9 Hz, 1H), 7.08 (t, J=9.1 Hz, 2H), 4.90-4.77 (m, 2H), 4.56 (dd, J=11.4, 5.0 Hz, 1H), 2.80-2.68 (m, 1H), 2.68-2.60 (m, 1H), 2.48-2.29 (m, 4H), 1.32 (q, J=4.3 Hz, 2H).

### Example 212

### Synthesis of (2-(2,6-dioxopiperidin-3-yl)benzo[d]oxazol-6-yl)methyl (2-fluoro-5-(trifluoromethoxy)phenyl)carbamate

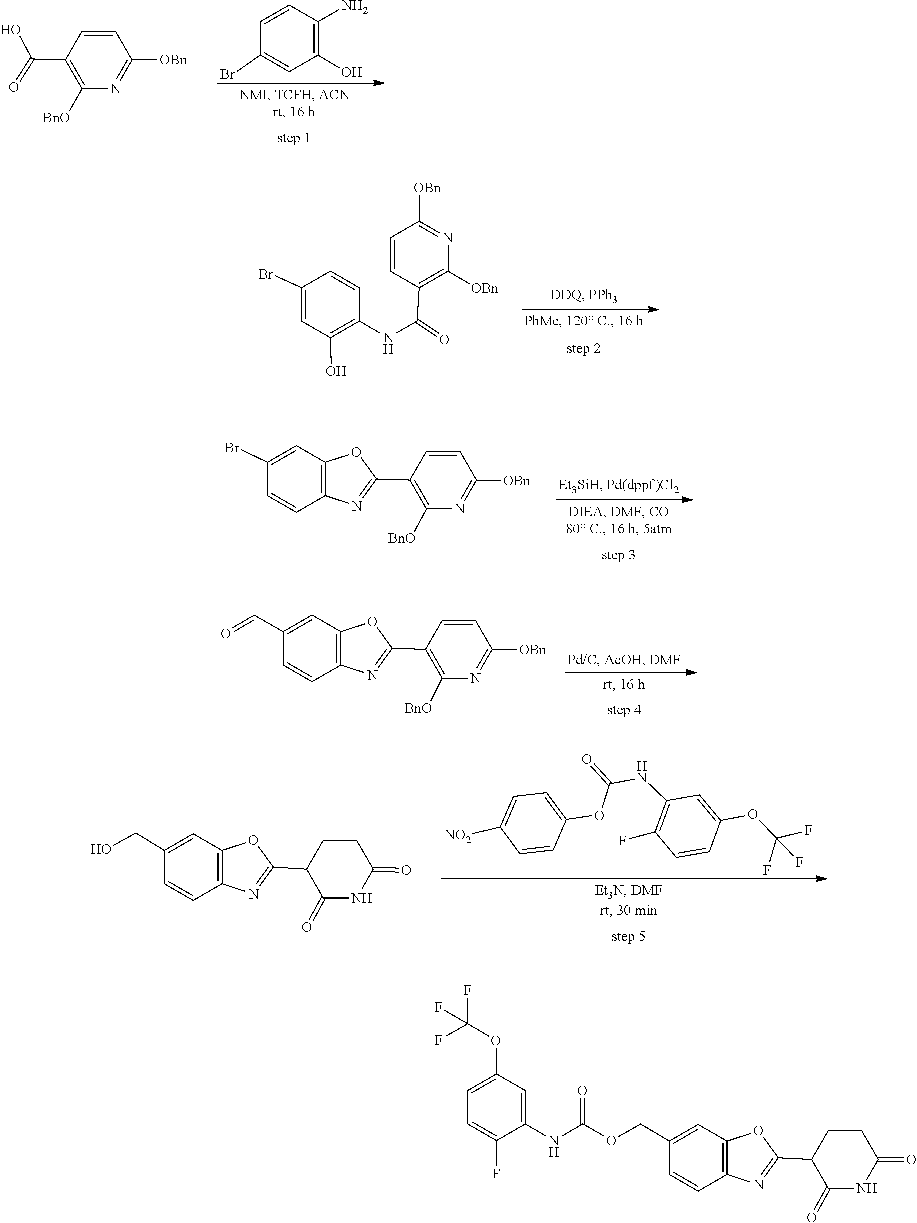

Step 1. Preparation of 2,6-bis(benzyloxy)-N-(4-bromo-2-hydroxyphenyl)nicotinamide. To a stirred solution of 2,6-bis(benzyloxy)pyridine-3-carboxylic acid (5 g, 14.909 mmol, 1 equiv) and NMI (3.6 g, 44.727 mmol, 3 equiv) in ACN (100 mL) were added 2-amino-5-bromophenol (4.2 g, 22.364 mmol, 1.5 equiv) and TCFH (6.2 g, 22.364 mmol, 1.5 equiv) in portions at room temperature. The resulting mixture was stirred for 16 h at room temperature. The resulting mixture was diluted with water (300 mL). The resulting mixture was extracted with EtOAc (3×200 mL). The combined organic layers were washed with brine (3×300 ml), dried over anhydrous Na₂SO₄. After filtration, the filtrate was concentrated under reduced pressure. The residue was purified by reversed-phase flash chromatography to afford 2,6-bis(benzyloxy)-N-(4-bromo-2-hydroxyphenyl)nicotinamide (4.6 g, 61%) as off-white solid. LC-MS (ES, m/z)=505.05 [M+1]⁺.

Step 2. Preparation of 2-(2,6-bis(benzyloxy)pyridin-3-yl)-6-bromobenzo[d]oxazole. To a stirred solution of 2,6-bis(benzyloxy)-N-(4-bromo-2-hydroxyphenyl)nicotinamide (4.3 g, 8.509 mmol, 1 equiv) in toluene (80 mL) were added PPh₃ (6.7 g, 25.527 mmol, 3 equiv) and DDQ (5.7 g, 25.527 mmol, 3 equiv) at room temperature. The resulting mixture was stirred for 16 h at 120° C. The resulting mixture was diluted with water (500 mL). The resulting mixture was extracted with EtOAc (3×200 mL). The combined organic layers were washed with brine (3×300 mL), dried over anhydrous Na₂SO₄. After filtration, the filtrate was concentrated under reduced pressure. The residue was purified by silica gel column chromatography, eluted with PE/EA (8:1) to afford 2-(2,6-bis(benzyloxy)pyridin-3-yl)-6-bromobenzo[d]oxazole (1.4 g, 34%) as yellow solid. LC-MS (ES, m/z)=487.05 [M+1]⁺.

Step 3. Preparation of 2-(2,6-bis(benzyloxy)pyridin-3-yl)benzo[d]oxazole-6-carbaldehyde. To a stirred solution of 2-(2,6-bis(benzyloxy)pyridin-3-yl)-6-bromobenzo[d]oxazole (1.3 g, 2.667 mmol, 1 equiv) and DIEA (1.7 g, 13.335 mmol, 5 equiv) in DMF (26 mL) were added Et₃SiH (2.4 g, 21.336 mmol, 8 equiv) and Pd(dppf)Cl₂ (0.4 g, 0.533 mmol, 0.2 equiv) at room temperature. The resulting mixture was stirred for 16 h at 80° C. under carbon monoxide (5 atm) atmosphere. The resulting mixture was diluted with water (200 mL). The resulting mixture was extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)benzo[d]oxazole-6-carbaldehyde (740 mg, 64%) as brown solid. LC-MS (ES, m/z)=437.25 [M+1]⁺.

Step 4. Preparation of 3-(6-(hydroxymethyl)benzo[d]oxazol-2-yl)piperidine-2,6-dione. To a stirred solution of 2-(2,6-bis(benzyloxy)pyridin-3-yl)benzo[d]oxazole-6-carbaldehyde (640 mg, 1.466 mmol, 1 equiv) and AcOH (264.1 mg, 4.398 mmol, 3 equiv) in DMF (10 mL) were added Pd/C (1.2 g, 10% on carbon (wetted with ca. 55% water)) at room temperature. The resulting mixture was stirred for 16 h at room temperature under hydrogen atmosphere. The resulting mixture was diluted with water (50 mL). The resulting mixture was extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(6-(hydroxymethyl)benzo[d]oxazol-2-yl)piperidine-2,6-dione (100 mg, 26%) as brown solid. LC-MS (ES, m/z)=261.10 [M+1]⁺.

Step 5. Preparation of (2-(2,6-dioxopiperidin-3-yl)benzo[d]oxazol-6-yl)methyl (2-fluoro-5-(trifluoromethoxy)phenyl)carbamate. To a stirred solution of 3-(6-(hydroxymethyl)benzo[d]oxazol-2-yl)piperidine-2,6-dione (50 mg, 0.192 mmol, 1 equiv) and TEA (58.3 mg, 0.576 mmol, 3 equiv) in DMF (0.5 mL) were added 4-nitrophenyl N-[2-fluoro-5-(trifluoromethoxy)phenyl]carbamate (89.9 mg, 0.250 mmol, 1.3 equiv) in portions at room temperature. The resulting mixture was stirred for 30 min at room temperature. The resulting mixture was diluted with water (20 mL). The resulting mixture was diluted with DCM (2×10 mL). The resulting mixture was washed with brine (2×20 mL), dried over anhydrous Na₂SO₄. After filtration, the filtrate was concentrated under reduced pressure. The crude product (50 mg) was purified by Prep-HPLC to afford (2-(2,6-dioxopiperidin-3-yl)benzo[d]oxazol-6-yl)methyl (2-fluoro-5-(trifluoromethoxy)phenyl)carbamate (21 mg, 23%) as white solid. LC-MS (ES, m/z)=482.10 [M+1]⁺. ¹H NMR (400 MHz, DMSO-d₆) δ 11.13 (s, 1H), 9.86 (s, 1H), 7.83 (t, J=3.3 Hz, 2H), 7.76 (d, J=8.2 Hz, 1H), 7.47 (dd, J=8.2, 1.5 Hz, 1H), 7.38 (dd, J=10.4, 9.0 Hz, 1H), 7.17-7.09 (m, 1H), 5.32 (s, 2H), 4.52 (dd, J=11.2, 5.0 Hz, 1H), 2.79-2.58 (m, 2H), 2.49-2.37 (m, 1H), 2.37-2.26 (m, 1H).

### Example 213

### Synthesis of 2-(2,6-dioxopiperidin-3-yl)-1-oxo-N-(6-(4-(trifluoromethoxy)phenyl)spiro[3.3]hept-5-en-2-yl)isoindoline-5-carboxamide

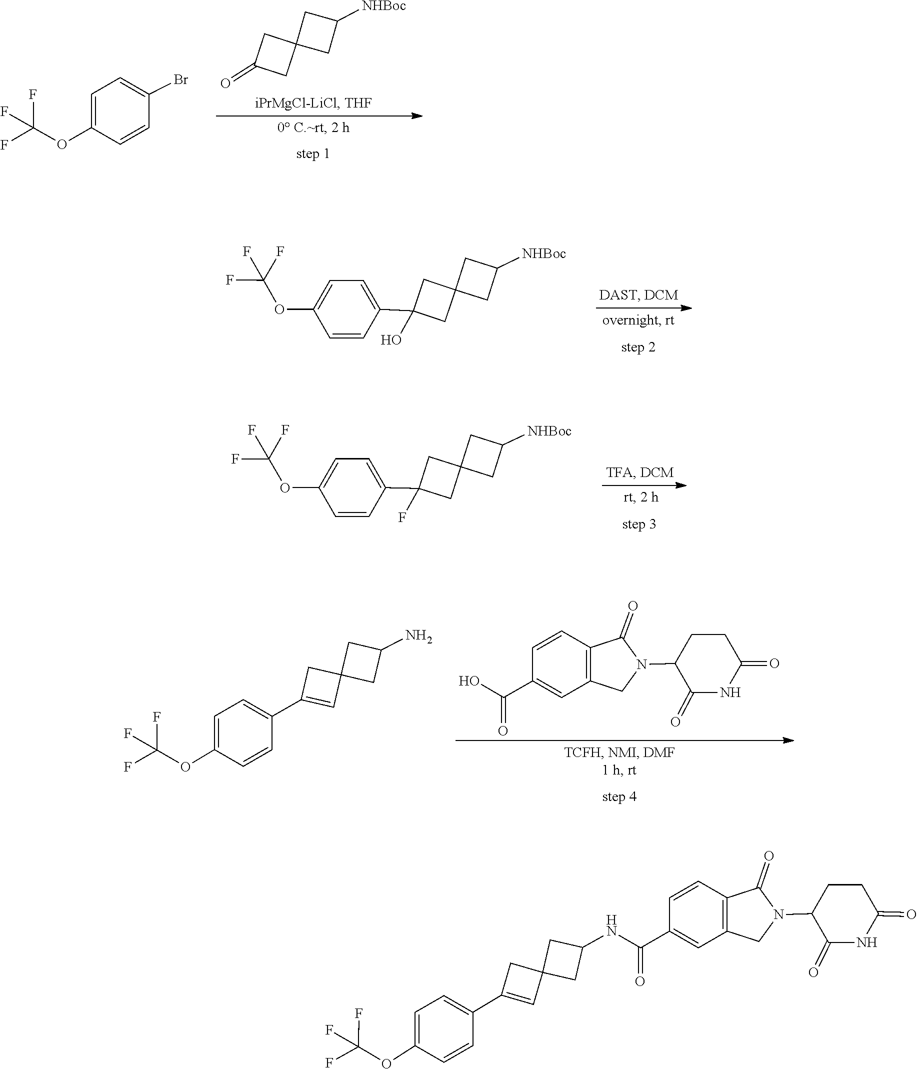

Step 1. Preparation of tert-butyl (6-hydroxy-6-(4-(trifluoromethoxy)phenyl)spiro[3.3]heptan-2-yl)carbamate. To a stirred solution of 1-bromo-4-(trifluoromethoxy)benzene (641.9 mg, 2.663 mmol, 1.20 equiv) in THF (8 mL) was added isopropylmagnesium chloride-Lithium chloride complex (2.1 mL, 2.730 mmol, 1.23 equiv) dropwise at 0° C. under nitrogen atmosphere. The resulting mixture was stirred for 1 h at 0° C. under nitrogen atmosphere. To the above mixture was added tert-butyl N-{6-oxospiro[3.3]heptan-2-yl}carbamate (500 mg, 2.219 mmol, 1 equiv) in THF (2 mL) dropwise at 0° C. The resulting mixture was stirred for an additional 1 h at room temperature. The reaction was quenched with sat. NH₄Cl (aq.) at 0° C. and extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford tert-butyl (6-hydroxy-6-(4-(trifluoromethoxy)phenyl)spiro[3.3]heptan-2-yl)carbamate (100 mg, 12%) as an off-white solid. ¹H NMR (400 MHz, DMSO-d₆) δ 7.55-7.46 (m, 2H), 7.33-7.25 (m, 2H), 7.04 (d, J=7.9 Hz, 1H), 5.48 (s, 1H), 3.80 (q, J=8.1 Hz, 1H), 2.39 (ddd, J=25.4, 12.2, 2.5 Hz, 3H), 2.25 (dd, J=12.4, 2.7 Hz, 1H), 2.11 (dt, J=12.4, 6.4 Hz, 1H), 2.04-1.96 (m, 1H), 1.84 (dd, J=10.8, 8.8 Hz, 1H), 1.37 (d, J=1.6 Hz, 1H), 1.36 (s, 9H).

Step 2. Preparation of tert-butyl (6-fluoro-6-(4-(trifluoromethoxy)phenyl)spiro[3.3]heptan-2-yl)carbamate. To a stirred solution of tert-butyl (6-hydroxy-6-(4-(trifluoromethoxy)phenyl)spiro[3.3]heptan-2-yl)carbamate (80 mg, 0.207 mmol, 1 equiv) in DCM (2 mL) was added DAST (0.07 mL, 0.517 mmol, 2.5 equiv) dropwise at 0° C. under nitrogen atmosphere. The resulting mixture was stirred overnight at room temperature under nitrogen atmosphere. The reaction was quenched with sat. NH₄Cl (aq.) at 0° C. and extracted with DCM (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl (6-fluoro-6-(4-(trifluoromethoxy)phenyl)spiro[3.3]heptan-2-yl)carbamate (65 mg, 81%) as a brown oil. LC-MS (ES, m/z)=375.15 [M-t-Bu+ACN+1]⁺.

Step 3. Preparation of 6-(4-(trifluoromethoxy)phenyl)spiro[3.3]hept-5-en-2-amine. To a stirred solution of tert-butyl (6-fluoro-6-(4-(trifluoromethoxy)phenyl)spiro[3.3]heptan-2-yl)carbamate (60 mg, 0.154 mmol, 1 equiv) in DCM (1 mL) was added trifluoroacetaldehyde (0.2 mL) dropwise at 0° C. under nitrogen atmosphere. The resulting mixture was stirred for 2 h at room temperature under nitrogen atmosphere. The resulting mixture was concentrated under vacuum to afford 6-(4-(trifluoromethoxy)phenyl)spiro[3.3]hept-5-en-2-amine (40 mg, crude) as a brown oil. The crude product was used in the next step directly without further purification. LC-MS (ES, m/z)=311.15 [M+ACN+1]⁺.

Step 4. Preparation of 2-(2,6-dioxopiperidin-3-yl)-1-oxo-N-(6-(4-(trifluoromethoxy)phenyl)spiro[3.3]hept-5-en-2-yl)isoindoline-5-carboxamide. To a stirred mixture of 2-(2,6-dioxopiperidin-3-yl)-1-oxo-3H-isoindole-5-carboxylic acid (32.1 mg, 0.111 mmol, 1.00 equiv) and 6-(4-(trifluoromethoxy)phenyl)spiro[3.3]hept-5-en-2-amine (17.3 mg, 0.078 mmol, 1.50 equiv) in DMF (0.6 mL) were added NMI (45.8 mg, 0.558 mmol, 5.01 equiv) and TCFH (46.9 mg, 0.167 mmol, 1.50 equiv) at room temperature under air atmosphere. The resulting mixture was stirred for 1 h at room temperature under nitrogen atmosphere. The reaction was quenched with water at 0° C. and extracted with DCM (3×20 mL). The combined organic layers were washed with brine (2×50 mL), dried over anhydrous Na₂SO₄. After filtration, the filtrate was concentrated under reduced pressure. The resulting mixture was purified by reversed-phase flash chromatography to afford crude product (15 mg). The crude product was purified by Prep-HPLC to afford 2-(2,6-dioxopiperidin-3-yl)-1-oxo-N-(6-(4-(trifluoromethoxy)phenyl)spiro[3.3]hept-5-en-2-yl)isoindoline-5-carboxamide (3 mg, 5%) as an off-white solid. LC-MS (ES, m/z)=581.30 [M+1]⁺. ¹H NMR (400 MHz, DMSO-d₆) δ 11.01 (s, 1H), 8.86 (t, J=6.2 Hz, 1H), 8.07 (s, 1H), 7.99 (d, J=8.2 Hz, 1H), 7.82 (d, J=7.8 Hz, 1H), 7.49 (t, J=8.4 Hz, 2H), 7.35 (d, J=8.2 Hz, 2H), 6.48 (s, 1H), 5.15 (dd, J=13.5, 5.2 Hz, 1H), 4.52 (d, J=17.4 Hz, 2H), 4.40 (d, J=17.6 Hz, 1H), 2.98-2.74 (m, 3H), 2.63 (s, 2H), 2.38 (s, 4H), 2.06-1.94 (m, 2H).

### Example 214

### Synthesis of N-(6-(2,4-difluorophenyl spiro[3.3]hept-5-en-2-yl)-2-(2,6-dioxopiperidin-3-yl oxoisoindoline-5-carboxamide

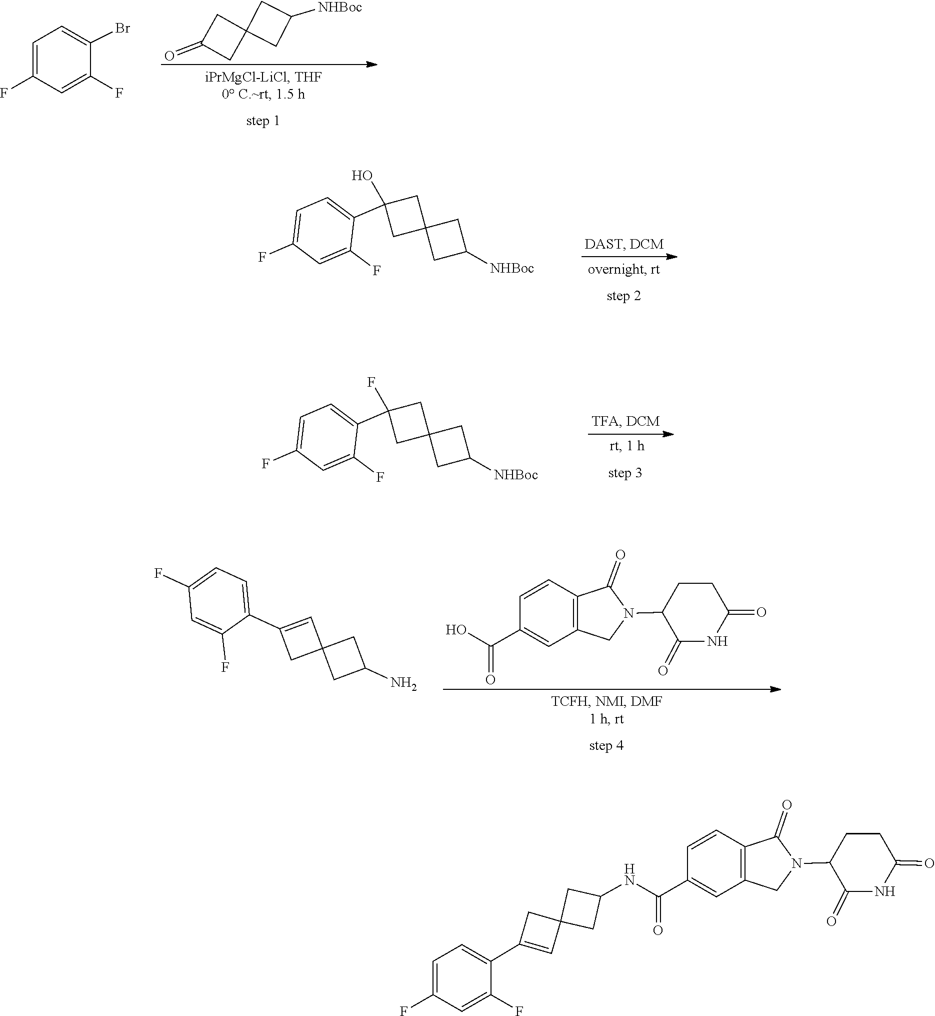

Step 1. Preparation of tert-butyl (6-(2,4-difluorophenyl)-6-hydroxyspiro[3.3]heptan-2-yl)carbamate. To a stirred solution of 1-bromo-2,4-difluorobenzene (856.6 mg, 4.439 mmol, 2.00 equiv) in THF (10 mL) was added isopropylmagnesium chloride-Lithium chloride complex (4.27 mL, 1.3 M in THF) dropwise at 0° C. under nitrogen atmosphere. The resulting mixture was stirred for 1 h at room temperature under nitrogen atmosphere. To the above mixture was added tert-butyl N-{6-oxospiro[3.3]heptan-2-yl}carbamate (500 mg, 2.219 mmol, 1 equiv) in THF (10 mL) dropwise at 0° C. The resulting mixture was stirred for an additional 30 min at room temperature. The reaction was quenched with sat. NH₄Cl (aq.) at 0° C. and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl (6-(2,4-difluorophenyl)-6-hydroxyspiro[3.3]heptan-2-yl)carbamate (430 mg, 57%) as an off-white solid. LC-MS (ES, m/z)=325.30 [M-t-Bu+ACN+1]⁺.

Step 2. Preparation of tert-butyl (6-(2,4-difluorophenyl)-6-fluorospiro[3.3]heptan-2-yl)carbamate. To a stirred solution of tert-butyl (6-(2,4-difluorophenyl)-6-hydroxyspiro[3.3]heptan-2-yl)carbamate (200 mg, 0.589 mmol, 1 equiv) in DCM (4 mL) was added DAST (0.19 mL, 1.472 mmol, 2.5 equiv) dropwise at 0° C. under nitrogen atmosphere. The resulting mixture was stirred overnight at room temperature under nitrogen atmosphere. The reaction was quenched by the addition of sat. NH₄Cl (aq.) (2 mL) at 0° C. and extracted with CH₂Cl₂ (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl (6-(2,4-difluorophenyl)-6-fluorospiro[3.3]heptan-2-yl)carbamate (180 mg, 89%) as an off-white solid. LC-MS (ES, m/z)=327.15 [M-t-Bu+ACN+1]⁺.

Step 3. Preparation of 6-(2,4-difluorophenyl)spiro[3.3]hept-5-en-2-amine. To a stirred solution of tert-butyl (6-(2,4-difluorophenyl)-6-fluorospiro[3.3]heptan-2-yl)carbamate (180 mg, 0.527 mmol, 1 equiv) in DCM (4 mL) was added TFA (0.8 mL) dropwise at 0° C. under nitrogen atmosphere. The resulting mixture was stirred for 1 h at room temperature under nitrogen atmosphere. The resulting mixture was concentrated under vacuum to afford 6-(2,4-difluorophenyl)spiro[3.3]hept-5-en-2-amine (130 mg, crude) as a brown oil. The crude product was used in the next step directly without further purification. LC-MS (ES, m/z)=263.15 [M+ACN+1]⁺.

Step 4. Preparation of N-(6-(2,4-difluorophenyl)spiro[3.3]hept-5-en-2-yl)-2-(2,6-dioxopiperidin-3-yl)-1-oxoisoindoline-5-carboxamide. To a stirred mixture of 2-(2,6-dioxopiperidin-3-yl)-1-oxo-3H-isoindole-5-carboxylic acid (100 mg, 0.347 mmol, 1.00 equiv) and 6-(2,4-difluorophenyl)spiro[3.3]hept-5-en-2-amine (115.1 mg, 0.520 mmol, 1.50 equiv) in DMF (2 mL) were added NMI (142.7 mg, 1.738 mmol, 5.01 equiv) and TCFH (146.0 mg, 0.520 mmol, 1.50 equiv) at room temperature under air atmosphere. The resulting mixture was stirred for 1 h at room temperature under nitrogen atmosphere. The resulting mixture was diluted with water (20 mL) and extracted with EtOAc (3×20 mL). The combined organic layers were washed with brine (2×30 mL), dried over anhydrous Na₂SO₄. After filtration, the filtrate was concentrated under reduced pressure. The residue was purified by reversed-phase flash chromatography to afford N-(6-(2,4-difluorophenyl)spiro[3.3]hept-5-en-2-yl)-2-(2,6-dioxopiperidin-3-yl)-1-oxoisoindoline-5-carboxamide (28 mg, 17%) as an off-white solid. LC-MS (ES, m/z)=492.20 [M+1]⁺. ¹H NMR (400 MHz, DMSO-d₆) δ 11.01 (s, 1H), 8.86 (dd, J=7.4, 1.9 Hz, 1H), 8.08 (s, 1H), 8.00 (dd, J=7.9, 1.7 Hz, 1H), 7.82 (d, J=7.9 Hz, 1H), 7.43-7.22 (m, 2H), 7.10 (tdd, J=8.1, 5.4, 2.5 Hz, 1H), 6.36 (d, J=2.9 Hz, 1H), 5.15 (ddd, J=13.3, 5.1, 1.9 Hz, 1H), 4.58-4.36 (m, 3H), 2.99-2.86 (m, 2H), 2.81 (d, J=2.0 Hz, 1H), 2.62 (ddd, J=17.1, 4.5, 2.2 Hz, 1H), 2.54 (d, J=2.2 Hz, 1H), 2.46-2.33 (m, 3H), 2.04 (dtd, J=10.8, 5.6, 3.0 Hz, 1H).

### Example 215

### Synthesis of N-(6-(3,4-difluorophenyl)spiro[3.3]hept-5-en-2-yl)-2-(2,6-dioxopiperidin-3-yl)-1-oxoisoindoline-5-carboxamide

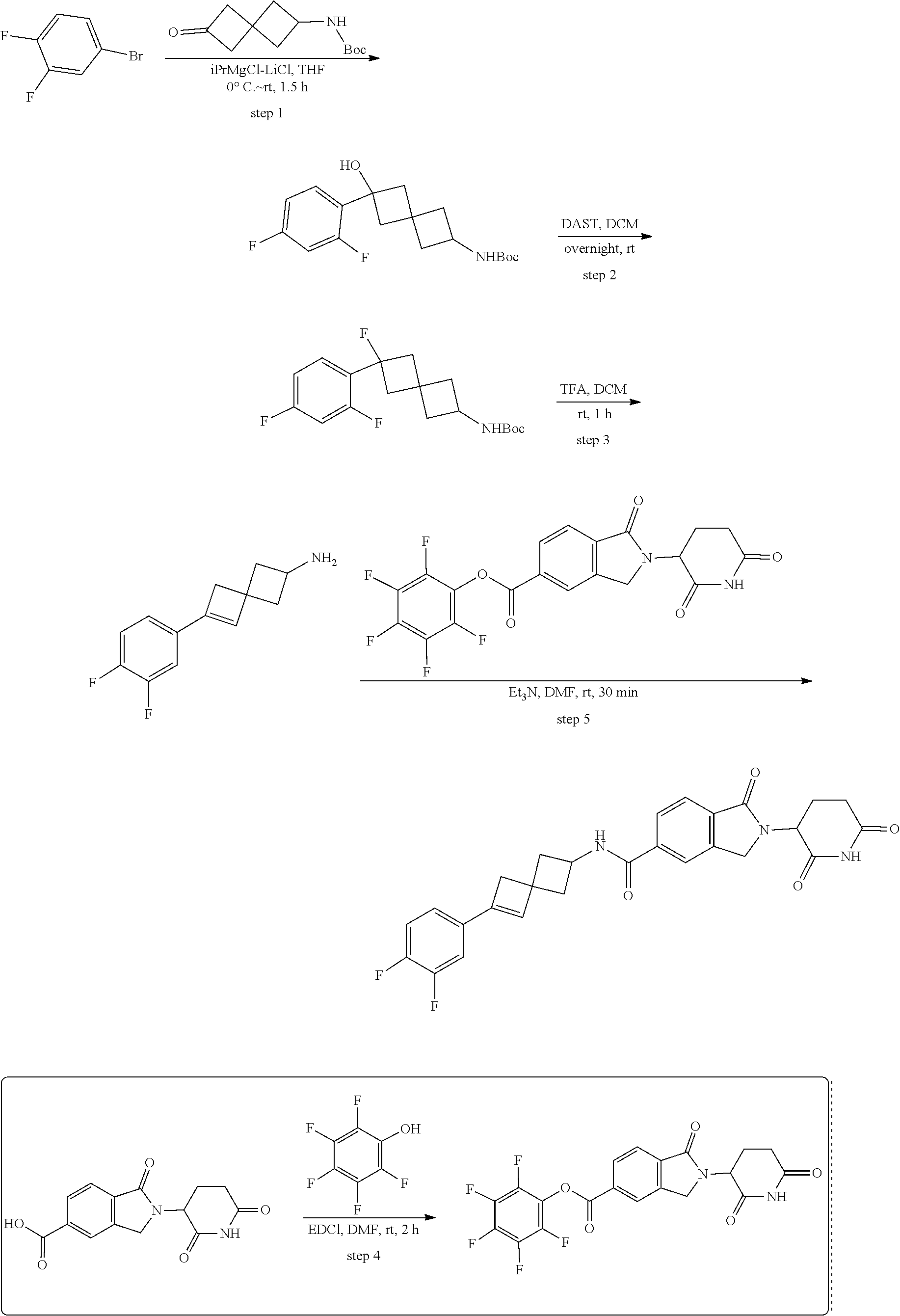

Step 1. Preparation of tert-butyl (6-(3,4-difluorophenyl)-6-hydroxyspiro[3.3]heptan-2-yl)carbamate. A solution of 4-bromo-1,2-difluorobenzene (642.5 mg, 3.329 mmol, 1.5 equiv) in THF (7.5 mL) was treated with isopropylmagnesium chloride-lithium chloride complex (3.3 mL, 1.3M in THF) for 1 h at room temperature under nitrogen atmosphere followed by the addition of tert-butyl N-{6-oxospiro[3.3]heptan-2-yl}carbamate (500 mg, 2.219 mmol, 1 equiv) in THF dropwise at 0° C. The resulting mixture was stirred for 1 h at room temperature under nitrogen atmosphere. The reaction was quenched with Water/Ice at 0° C. The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl (6-(3,4-difluorophenyl)-6-hydroxyspiro[3.3]heptan-2-yl)carbamate (300 mg, 37%) as a yellow oil. LC-MS (ES, m/z)=266.10 [M-tBu+Na]⁺.

Step 2. Preparation of tert-butyl (6-(3,4-difluorophenyl)-6-fluorospiro[3.3]heptan-2-yl)carbamate. To a stirred mixture of tert-butyl N-[6-(3,4-difluorophenyl)-6-hydroxyspiro[3.3]heptan-2-yl]carbamate (300 mg, 0.884 mmol, 1 equiv) in DCM (4.5 mL) was added DAST (284.97 mg, 1.768 mmol, 2 equiv) dropwise at 0° C. under nitrogen atmosphere. The resulting mixture was stirred for 2 h at room temperature under nitrogen atmosphere. The reaction was quenched with sat. NH₄Cl (aq.) at 0° C. The resulting mixture was extracted with CH₂Cl₂ (3×30 mL). The combined organic layers were washed with brine (3×50 mL), dried over anhydrous Na₂SO₄. After filtration, the filtrate was concentrated under reduced pressure. The residue was purified by reversed-phase flash chromatography to afford tert-butyl (6-(3,4-difluorophenyl)-6-fluorospiro[3.3]heptan-2-yl)carbamate (170 mg, 56%) as a yellow oil. LC-MS (ES, m/z)=327.10 [M-tBu+ACN+1]⁺.

Step 3. Preparation of 6-(3,4-difluorophenyl)spiro[3.3]hept-5-en-2-amine. To a stirred mixture of tert-butyl (6-(3,4-difluorophenyl)-6-fluorospiro[3.3]heptan-2-yl)carbamate (130 mg, 0.405 mmol, 1 equiv) in DCM (2 mL) was added TFA (0.4 mL, 5.385 mmol, 13.31 equiv) dropwise at 0° C. under air atmosphere. The resulting mixture was stirred for 2 h at room temperature under the air atmosphere. The resulting mixture was concentrated under vacuum. This resulted in 6-(3,4-difluorophenyl)spiro[3.3]hept-5-en-2-amine (140 mg, crude) as a brown solid. The residue was used for the next step without further purification. LC-MS (ES, m/z)=263.10 [M+ACN+1]⁺.

Step 4. Preparation of perfluorophenyl 2-(2,6-dioxopiperidin-3-yl)-1-oxoisoindoline-5-carboxylate. To a stirred mixture of 2-(2,6-dioxopiperidin-3-yl)-1-oxo-3H-isoindole-5-carboxylic acid (1 g, 3.469 mmol, 1 equiv) and pentafluorophenol (0.77 g, 4.163 mmol, 1.2 equiv) in DMF (15 mL) was added EDCI (1.00 g, 5.204 mmol, 1.5 equiv) in portions at 0° C. under nitrogen atmosphere. The resulting mixture was stirred for 2 h at room temperature under nitrogen atmosphere. The resulting mixture was diluted with water and extracted with EtOAc (3×50 mL). The combined organic layers were washed with brine (3×100 mL), dried over anhydrous Na₂SO₄. After filtration, the filtrate was concentrated underreduced pressure. The residue was purified by silica gel column chromatography, eluted with PE/EA (1:1) to afford perfluorophenyl 2-(2,6-dioxopiperidin-3-yl)-1-oxoisoindoline-5-carboxylate (900 mg, 57%) as an off-white solid. LC-MS (ES, m/z)=455.15 [M+1]⁺.

Step 5. Preparation of N-(6-(3,4-difluorophenyl)spiro[3.3]hept-5-en-2-yl)-2-(2,6-dioxopiperidin-3-yl)-1-oxoisoindoline-5-carboxamide. A solution of 6-(3,4-difluorophenyl)spiro[3.3]hept-5-en-2-amine (40 mg, 0.181 mmol, 1 equiv) in DMF (1 mL) was treated with Et₃N (54.9 mg, 0.543 mmol, 3 equiv) at room temperature under nitrogen atmosphere followed by the addition of perfluorophenyl 2-(2,6-dioxopiperidin-3-yl)-1-oxoisoindoline-5-carboxylate (98.6 mg, 0.217 mmol, 1.2 equiv) at 0° C. The resulting mixture was stirred for 30 min at room temperature under the air atmosphere. The reaction was quenched with sat. NH₄Cl (aq.) at 0° C. The resulting mixture was extracted with CH₂Cl₂ (3×20 mL). The combined organic layers were washed with brine (3×30 mL), dried over anhydrous Na₂SO₄. After filtration, the filtrate was concentrated under reduced pressure. The residue was purified by Prep-HPLC to afford N-(6-(3,4-difluorophenyl)spiro[3.3]hept-5-en-2-yl)-2-(2,6-dioxopiperidin-3-yl)-1-oxoisoindoline-5-carboxamide (18 mg, 20%) as off-white solid. LC-MS (ES, m/z)=492.20 [M+1]⁺. ¹H NMR (400 MHz, DMSO-d₆) δ 10.84 (s, 1H), 8.85 (dd, J=7.4, 5.9 Hz, 1H), 8.07 (s, 1H), 8.03-7.95 (m, 1H), 7.82 (d, J=8.0 Hz, 1H), 7.50-7.36 (m, 2H), 7.23-7.14 (m, 1H), 6.47 (s, 1H), 5.15 (dd, J=13.3, 5.1 Hz, 1H), 4.58-4.35 (m, 3H), 2.98-2.82 (m, 2H), 2.74 (s, 1H), 2.62 (ddd, J=17.3, 4.5, 2.3 Hz, 1H), 2.49-2.43 (m, 3H), 2.38 (qd, J=10.3, 9.1, 3.6 Hz, 2H), 2.10-1.99 (m, 1H).

### Example 216

### Synthesis of N-(6-(4-cyanophenyl)spiro[3.3]hept-5-en-2-yl)-2-(2,6-dioxopiperidin-3-yl)-1-oxoisoindoline-5-carboxamide

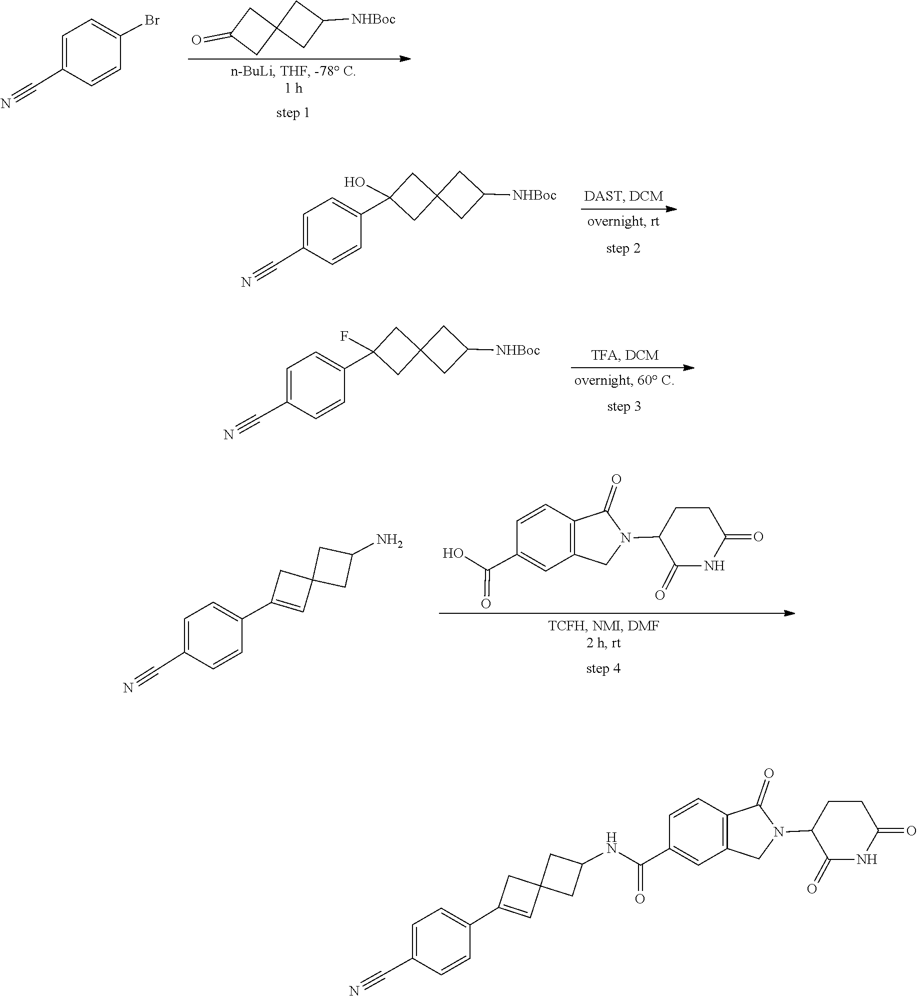

Step 1. Preparation of tert-butyl (6-(4-cyanophenyl)-6-hydroxyspiro[3.3]heptan-2-yl)carbamate. A solution of 4-bromobenzonitrile (1.01 g, 5.547 mmol, 2.5 equiv) in THF (10 mL) was treated with n-BuLi (2.2 mL, 5.547 mmol, 2.5 equiv) for 20 min at −78° C. under nitrogen atmosphere followed by the addition of tert-butyl N-{6-oxospiro[3.3]heptan-2-yl}carbamate (500 mg, 2.219 mmol, 1 equiv) at −78° C. The resulting mixture was stirred for 1 h at −78° C. under nitrogen atmosphere. The reaction was quenched by the addition of sat. NH₄Cl (aq.) (5 mL) at 0° C. and extracted with EtOAc (3×50 mL). The combined organic layers were washed with brine (2×30 mL), dried over anhydrous Na₂SO₄. After filtration, the filtrate was concentrated under reduced pressure. The residue was purified by reversed-phase flash chromatography to afford tert-butyl (6-(4-cyanophenyl)-6-hydroxyspiro[3.3]heptan-2-yl)carbamate (570 mg, 78%) as a yellow oil. LC-MS (ES, m/z)=314.15 [M-tBu+ACN+1]⁺.

Step 2. Preparation of tert-butyl (6-(4-cyanophenyl)-6-fluorospiro[3.3]heptan-2-yl)carbamate. To a stirred mixture of tert-butyl (6-(4-cyanophenyl)-6-hydroxyspiro[3.3]heptan-2-yl)carbamate (370 mg, 1.127 mmol, 1 equiv) in DCM (5 mL) was added DAST (363.20 mg, 2.254 mmol, 2 equiv) at room temperature under nitrogen atmosphere. The resulting mixture was stirred overnight at room temperature under nitrogen atmosphere. The reaction was quenched by the addition of sat. NH₄Cl (aq.) at 0° C. and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl (6-(4-cyanophenyl)-6-fluorospiro[3.3]heptan-2-yl)carbamate (350 mg, 94%) as a yellow oil. LC-MS (ES, m/z)=297.30 [M-tBu+ACN+1]⁺.

Step 3. Preparation of 4-(6-aminospiro[3.3]hept-1-en-2-yl)benzonitrile. To a stirred mixture of tert-butyl (6-(4-cyanophenyl)-6-fluorospiro[3.3]heptan-2-yl)carbamate (200 mg, 0.605 mmol, 1 equiv) in DCM (4 mL) was added TFA (0.80 mL, 10.763 mmol, 17.79 equiv) dropwise at 0° C. under nitrogen atmosphere. The resulting mixture was stirred overnight at 60° C. under nitrogen atmosphere. The resulting mixture was concentrated under reduced pressure. The residue was purified by reversed-phase flash chromatography to afford 4-(6-aminospiro[3.3]hept-1-en-2-yl)benzonitrile (30 mg, 24%) as a white solid. LC-MS (ES, m/z)=211.10 [M+1]⁺.

Step 4. Preparation of N-(6-(4-cyanophenyl)spiro[3.3]hept-5-en-2-yl)-2-(2,6-dioxopiperidin-3-yl)-1-oxoisoindoline-5-carboxamide. To a stirred mixture of 4-(6-aminospiro[3.3]hept-1-en-2-yl)benzonitrile (15 mg, 0.071 mmol, 1.37 equiv) and 2-(2,6-dioxopiperidin-3-yl)-1-oxo-3H-isoindole-5-carboxylic acid (15 mg, 0.052 mmol, 1.00 equiv) in DMF (0.5 mL) were added TCFH (21.9 mg, 0.078 mmol, 1.5 equiv) and NMI (17.0 mg, 0.208 mmol, 4 equiv) at room temperature. The resulting mixture was stirred for 2 h at room temperature. The reaction was diluted with water (30 mL) at 0° C. and extracted with EtOAc (3×20 mL). The combined organic layers were washed with brine (2×50 mL), dried over anhydrous Na₂SO₄. The crude product (15 mg) was purified by Prep-HPLC to afford N-(6-(4-cyanophenyl)spiro[3.3]hept-5-en-2-yl)-2-(2,6-dioxopiperidin-3-yl)-1-oxoisoindoline-5-carboxamide (11 mg, 42%) as a white solid. LC-MS (ES, m/z)=481.20 [M+1]⁺. ¹H NMR (400 MHz, DMSO-d₆) δ 11.02 (s, 1H), 8.87 (dd, J=7.4, 4.7 Hz, 1H), 8.08 (s, 1H), 8.00 (d, J=8.0 Hz, 1H), 7.82 (dd, J=8.3, 2.9 Hz, 3H), 7.54 (t, J=8.5 Hz, 2H), 6.69 (s, 1H), 5.15 (dd, J=13.3, 5.1 Hz, 1H), 4.57-4.40 (m, 3H), 2.92 (q, J=5.4 Hz, 2H), 2.79 (s, 1H), 2.66-2.57 (m, 1H), 2.49-2.30 (m, 4H), 2.04 (dq, J=13.3, 4.9, 3.0 Hz, 1H).

### Example 217

### Synthesis of N-(6-(3,4-difluorophenyl)spiro[3.3]heptan-2-yl)-2-(2,6-dioxopiperidin-3-yl oxoisoindoline-5-carboxamide

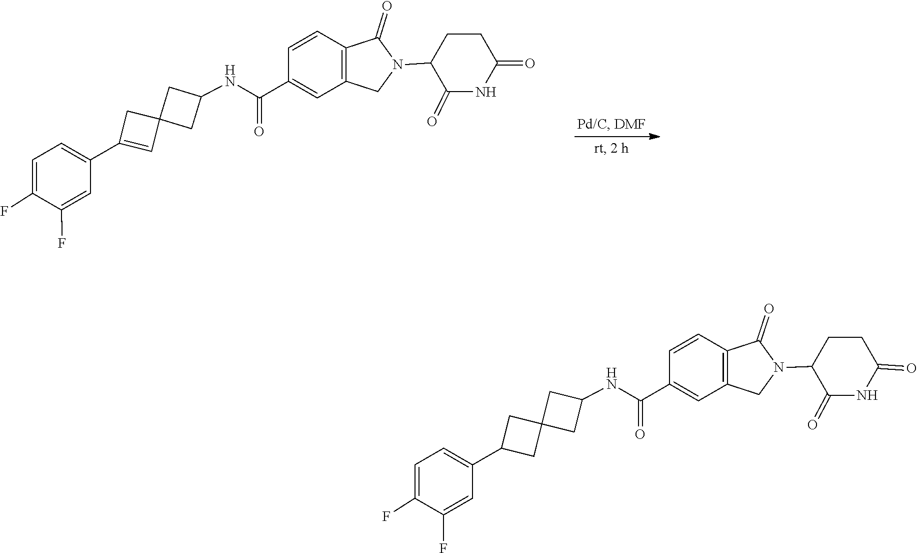 [SMILES: O=C1CCC(N2Cc3cc(C(=O)NC4CC5(C=C(c6ccc(F)c(F)c6)C5)C4)ccc3C2=O)C(=O)N1 ; O=C1CCC(N2Cc3cc(C(=O)NC4CC5(C4)CC(c4ccc(F)c(F)c4)C5)ccc3C2=O)C(=O)N1]

Step 1. Preparation of N-(6-(3,4-difluorophenyl)spiro[3.3]heptan-2-yl)-2-(2,6-dioxopiperidin-3-yl)-1-oxoisoindoline-5-carboxamide. A mixture of N-[6-(3,4-difluorophenyl)spiro[3.3]hept-5-en-2-yl]-2-(2,6-dioxopiperidin-3-yl)-1-oxo-3H-isoindole-5-carboxamide (60 mg, 0.122 mmol, 1 equiv) and Pd/C (50 mg, Palladium 10% on Carbon (wetted with ca. 55% Water)) in DMF (1.0 mL) was stirred for 2 h at room temperature under hydrogen atmosphere. The resulting mixture was filtered, the filter cake was washed with DMF (1×1 mL). The filtrate was concentrated under reduced pressure. The residue was purified by Prep-HPLC to afford N-(6-(3,4-difluorophenyl)spiro[3.3]heptan-2-yl)-2-(2,6-dioxopiperidin-3-yl)-1-oxoisoindoline-5-carboxamide (17 mg, 29%) as off-white solid. LC-MS (ES, m/z)=494.20 [M+1]⁺. ¹H NMR (400 MHz, DMSO-d₆) δ 11.00 (s, 1H), 8.78 (d, J=7.4 Hz, 1H), 8.05 (s, 1H), 7.97 (dd, J=8.0, 1.5 Hz, 1H), 7.80 (d, J=8.0 Hz, 1H), 7.42-7.23 (m, 2H), 7.06 (ddd, J=9.2, 4.4, 2.2 Hz, 1H), 5.14 (dd, J=13.3, 5.1 Hz, 1H), 4.51 (d, J=17.5 Hz, 1H), 4.46-4.25 (m, 2H), 3.42 (p, J=8.9 Hz, 1H), 2.92 (ddd, J=17.2, 13.6, 5.4 Hz, 1H), 2.68-2.53 (m, 2H), 2.49-2.34 (m, 2H), 2.35-1.95 (m, 7H).

### Example 218

### Synthesis of N-(6-(2,4-difluorophenyl)spiro[3.3]heptan-2-yl)-2-(2,6-dioxopiperidin-3-yl oxoisoindoline-5-carboxamide

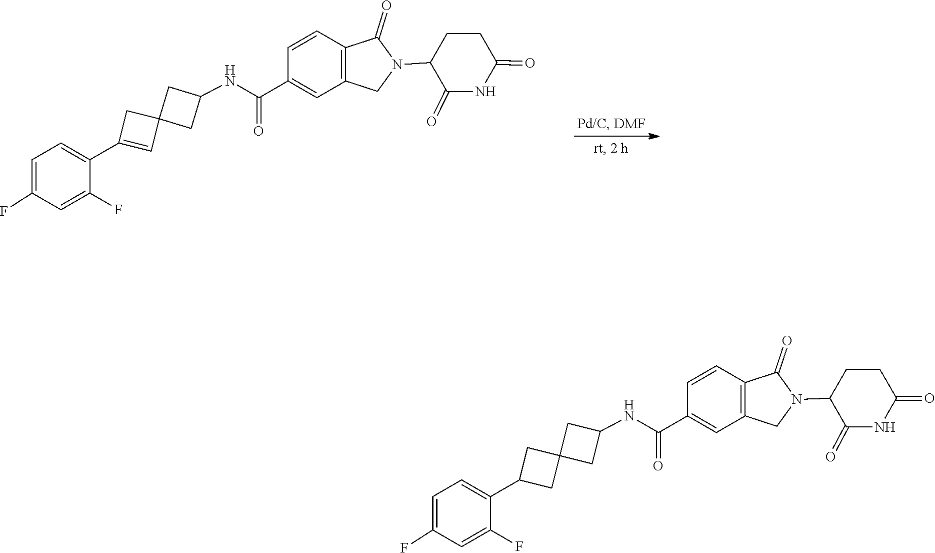 [SMILES: O=C1CCC(N2Cc3cc(C(=O)NC4CC5(C=C(c6ccc(F)cc6F)C5)C4)ccc3C2=O)C(=O)N1 ; O=C1CCC(N2Cc3cc(C(=O)NC4CC5(C4)CC(c4ccc(F)cc4F)C5)ccc3C2=O)C(=O)N1]

Step 1. Preparation of N-(6-(2,4-difluorophenyl)spiro[3.3]heptan-2-yl)-2-(2,6-dioxopiperidin-3-yl)-1-oxoisoindoline-5-carboxamide. To a solution of N-[6-(2,4-difluorophenyl)spiro[3.3]hept-5-en-2-yl]-2-(2,6-dioxopiperidin-3-yl)-1-oxo-3H-isoindole-5-carboxamide (40 mg, 0.081 mmol, 1 equiv) in DMF (0.8 mL) was added Pd/C (20 mg, Palladium 10% on Carbon (wetted with ca. 60% Water)) under nitrogen atmosphere in a 10 mL round-bottom flask. The mixture was hydrogenated at room temperature for 2 h under hydrogen atmosphere using a hydrogen balloon, The resulting mixture was filtered through a Celite pad, and the filter cake was washed with THF (3×5 mL). The filtrate was concentrated under reduced pressure. The residue was purified by reversed-phase flash chromatography to afford N-(6-(2,4-difluorophenyl)spiro[3.3]heptan-2-yl)-2-(2,6-dioxopiperidin-3-yl)-1-oxoisoindoline-5-carboxamide (17 mg, 42%) as an off-white solid. LC-MS (ES, m/z)=494.25 [M+1]⁺. ¹H NMR (400 MHz, DMSO-d₆) δ 11.01 (s, 1H), 8.78 (d, J=7.4 Hz, 1H), 8.06 (s, 1H), 7.97 (dd, J=8.0, 1.4 Hz, 1H), 7.80 (d, J=7.9 Hz, 1H), 7.36 (td, J=8.7, 6.5 Hz, 1H), 7.13 (ddd, J=10.7, 9.3, 2.6 Hz, 1H), 7.03 (td, J=8.6, 2.7 Hz, 1H), 5.14 (dd, J=13.3, 5.1 Hz, 1H), 4.52 (d, J=17.5 Hz, 1H), 4.44-4.30 (m, 2H), 3.53 (p, J=8.9 Hz, 1H), 2.92 (ddd, J=17.2, 13.6, 5.4 Hz, 1H), 2.69-2.54 (m, 2H), 2.48-2.36 (m, 1H), 2.35-1.97 (m, 7H).

### Example 219

### Synthesis of N-(6-(4-cyanophenyl)spiro[3.3]heptan-2-yl)-2-(2,6-dioxopiperidin-3-yl oxoisoindoline-5-carboxamide

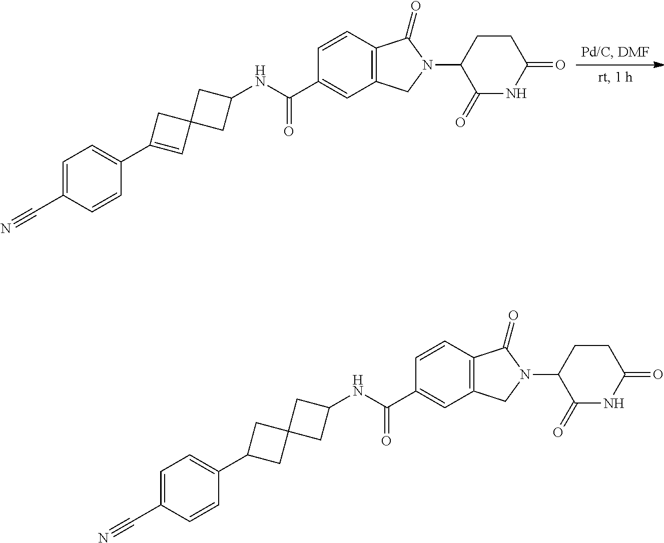 [SMILES: N#Cc1ccc(C2=CC3(C2)CC(NC(=O)c2ccc4c(c2)CN(C2CCC(=O)NC2=O)C4=O)C3)cc1 ; N#Cc1ccc(C2CC3(CC(NC(=O)c4ccc5c(c4)CN(C4CCC(=O)NC4=O)C5=O)C3)C2)cc1]

Step 1. Preparation of N-(6-(4-cyanophenyl)spiro[3.3]heptan-2-yl)-2-(2,6-dioxopiperidin-3-yl)-1-oxoisoindoline-5-carboxamide. To a stirred mixture of N-[6-(4-cyanophenyl)spiro[3.3]hept-5-en-2-yl]-2-(2,6-dioxopiperidin-3-yl)-1-oxo-3H-isoindole-5-carboxamide (9 mg, 0.019 mmol, 1 equiv) in DMF (0.3 mL) was added Pd/C (5 mg, Palladium 10% on Carbon (wetted with ca. 55% Water)) at room temperature under hydrogen atmosphere. The resulting mixture was stirred for 1 h at room temperature under hydrogen atmosphere. The resulting mixture was filtered, the filter cake was washed with THF (2×5 mL). The filtrate was concentrated under reduced pressure. The crude product (9 mg) was purified by Prep-HPLC to afford N-(6-(4-cyanophenyl)spiro[3.3]heptan-2-yl)-2-(2,6-dioxopiperidin-3-yl)-1-oxoisoindoline-5-carboxamide (7.7 mg, 85%) as a white solid. LC-MS (ES, m/z)=483.25 [M+1]⁺. ¹H NMR (400 MHz, DMSO-d₆) δ 11.01 (s, 1H), 8.78 (d, J=7.4 Hz, 1H), 8.05 (s, 1H), 7.99-7.80 (m, 2H), 7.80-7.73 (m, 2H), 7.43 (d, J=8.2 Hz, 2H), 5.14 (dd, J=13.3, 5.1 Hz, 1H), 4.51 (d, J=17.5 Hz, 1H), 4.47-4.32 (m, 2H), 3.51 (q, J=8.8 Hz, 1H), 2.90 (dd, J=12.8, 5.1 Hz, 1H), 2.61 (d, J=17.4 Hz, 2H), 2.40-1.97 (m, 9H).

### Examples 220 and 221

### Synthesis of 3-(6-(7,7-difluoro-2-azaspiro[3.5]nonane-2-carbonyl benzo[d]oxazol-2-yl)piperidine-2,6-dione and 3-(6-(7-fluoro-2-azaspiro[3.5]non-6-ene-2-carbonyl benzo[d]oxazol-2-yl)piperidine-2,6-dione

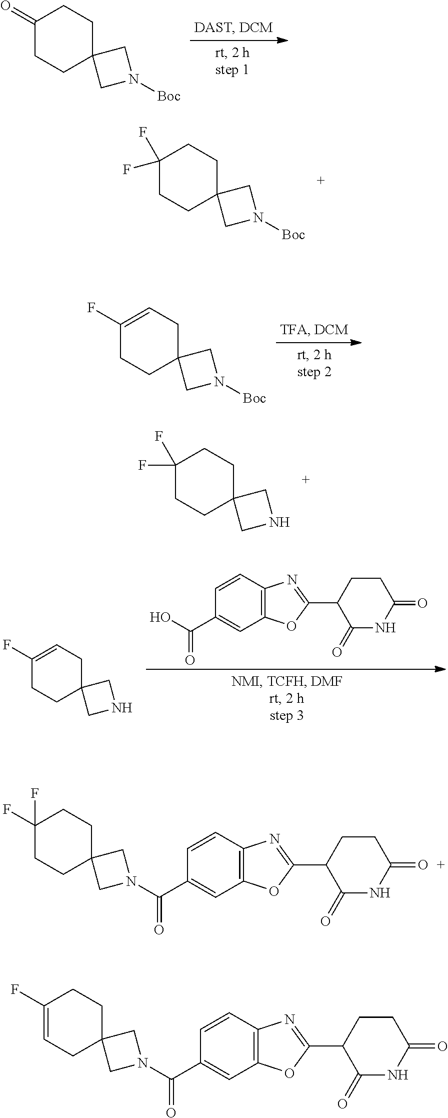

Step 1. Preparation of tert-butyl 7,7-difluoro-2-azaspiro[3.5]nonane-2-carboxylate and tert-butyl 7-fluoro-2-azaspiro[3.5]non-6-ene-2-carboxylate. To a stirred mixture of tert-butyl 7-oxo-2-azaspiro[3.5]nonane-2-carboxylate (500 mg, 2.089 mmol, 1 equiv) in DCM (7.5 mL) was added DAST (841.9 mg, 5.223 mmol, 2.5 equiv) dropwise at 0° C. under nitrogen atmosphere. The resulting mixture was stirred for 2 h at room temperature under nitrogen atmosphere. The reaction was quenched with sat. NH₄Cl (aq.) at 0° C. The resulting mixture was diluted with water (30 mL) and extracted with DCM (3×20 mL). The combined organic layers were washed with brine (2×20 mL), dried over anhydrous Na₂SO₄. After filtration, the filtrate was concentrated under reduced pressure. The residue was purified by reversed-phase flash chromatography to afford a mixture of tert-butyl 7,7-difluoro-2-azaspiro[3.5]nonane-2-carboxylate and tert-butyl 7-fluoro-2-azaspiro[3.5]non-6-ene-2-carboxylate (500 mg, 92%) as an orange solid. LC-MS (ES, m/z)=247.15 [M-tBu+ACN+1]⁺.

Step 2. Preparation of 7,7-difluoro-2-azaspiro[3.5]nonane and 7-fluoro-2-azaspiro[3.5]non-6-ene. A mixture of tert-butyl 7,7-difluoro-2-azaspiro[3.5]nonane-2-carboxylate and tert-butyl 7-fluoro-2-azaspiro[3.5]non-6-ene-2-carboxylate (500 mg, 1.913 mmol, 1 equiv) in DCM (5 mL) was added TFA (1 mL) dropwise at 0° C. under nitrogen atmosphere. The resulting mixture was stirred for 2 h at room temperature. The resulting mixture was concentrated under vacuum to afford a mixture of 7,7-difluoro-2-azaspiro[3.5]nonane and 7-fluoro-2-azaspiro[3.5]non-6-ene (700 mg, crude) as a brown oil. The residue was used in the next step directly without further purification. LC-MS (ES, m/z)=162.15 [M+1]⁺.

Step 3. Preparation of 3-(6-(7,7-difluoro-2-azaspiro[3.5]nonane-2-carbonyl)benzo[d]oxazol-2-yl)piperidine-2,6-dione and 3-(6-(7-fluoro-2-azaspiro[3.5]non-6-ene-2-carbonyl)benzo[d]oxazol-2-yl)piperidine-2,6-dione. A mixture of 7,7-difluoro-2-azaspiro[3.5]nonane and 7-fluoro-2-azaspiro[3.5]non-6-ene (96.99 mg, 0.602 mmol, 1.1 equiv), 2-(2,6-dioxopiperidin-3-yl)-1,3-benzoxazole-6-carboxylic acid (150 mg, 0.547 mmol, 1.00 equiv), NMI (179.6 mg, 2.188 mmol, 4 equiv) and TCFH (306.9 mg, 1.094 mmol, 2 equiv) in DMF (3 mL) was stirred for 2 h at room temperature under air atmosphere. The resulting mixture was filtered, the filter cake was washed with acetonitrile (3×5 mL). The filtrate was concentrated under reduced pressure. The crude product was purified by Prep-HPLC with the following conditions (Column: CHIRALPAK IE, 3*25 cm, 5 μm; Mobile Phase A: MtBE (0.1% DEA)-HPLC, Mobile Phase B: EtOH; Flow rate: 32 mL/min; Gradient: isocratic 30; Wave Length: 212/252 nm; RT1 (min): 25.95; RT2 (min): 34.64; Sample Solvent: EtOH:DCM=1:1-HPLC; Injection Volume: 1.35 mL; Number Of Runs: 4) to afford 3-(6-(7,7-difluoro-2-azaspiro[3.5]nonane-2-carbonyl)benzo[d]oxazol-2-yl)piperidine-2,6-dione (35 mg, 14%) and 3-(6-(7-fluoro-2-azaspiro[3.5]non-6-ene-2-carbonyl)benzo[d]oxazol-2-yl)piperidine-2,6-dione (5.7 mg, 2.57%) as an off-white solid.

Analytical data for 3-(6-(7,7-difluoro-2-azaspiro[3.5]nonane-2-carbonyl)benzo[d]oxazol-2-yl)piperidine-2,6-dione. LC-MS (ES, m/z)=418.15 [M+1]⁺. ¹H NMR (400 MHz, DMSO-d₆) δ 11.04 (s, 1H), 8.07-7.98 (m, 1H), 7.81 (d, J=8.3 Hz, 1H), 7.69 (dd, J=8.3, 1.5 Hz, 1H), 4.56 (dd, J=11.3, 5.1 Hz, 1H), 4.11 (s, 2H), 3.82 (s, 2H), 2.95-2.77 (m, 1H), 2.75-2.58 (m, 2H), 2.47-2.28 (m, 2H), 1.86 (dt, J=21.1, 6.4 Hz, 9H), 1.27-1.09 (m, 2H).

Analytical data for 3-(6-(7-fluoro-2-azaspiro[3.5]non-6-ene-2-carbonyl)benzo[d]oxazol-2-yl)piperidine-2,6-dione. LC-MS (ES, m/z)=398.20 [M+1]⁺. ¹H NMR (400 MHz, DMSO-d₆) δ 11.15 (s, 1H), 8.01 (d, J=1.4 Hz, 1H), 7.81 (d, J=8.3 Hz, 1H), 7.69 (dd, J=8.3, 1.6 Hz, 1H), 5.15 (dt, J=17.0, 3.9 Hz, 1H), 4.56 (dd, J=11.3, 5.1 Hz, 1H), 4.19-4.00 (m, 2H), 3.92-3.71 (m, 2H), 2.82-2.58 (m, 2H), 2.50-2.12 (m, 6H), 1.91 (dt, J=6.9, 3.5 Hz, 2H).

### Example 222

### Synthesis of 3-(1-oxo-5-trifluoromethyl-2-azaspiro[3.4]octane-2-carbonyl)isoindolin-2-yl)piperidine-2,6-dione

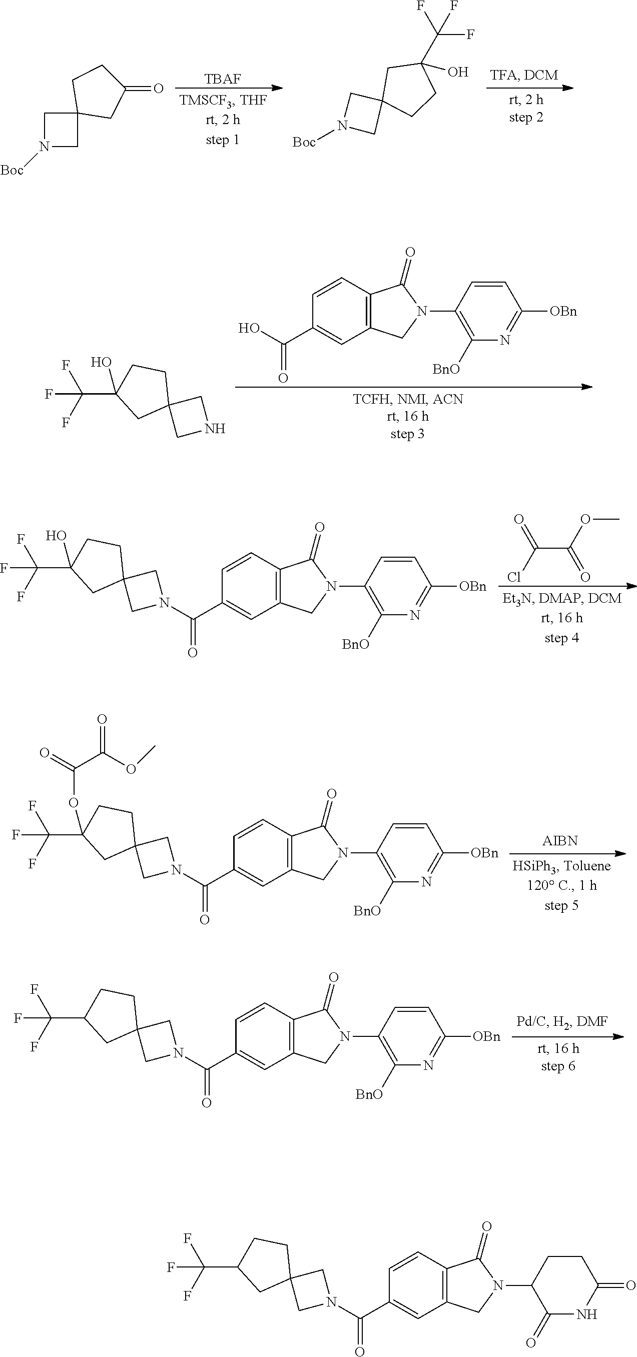

Step 1. Preparation of tert-butyl 6-hydroxy-6-(trifluoromethyl)-2-azaspiro[3.4]octane-2-carboxylate. To a stirred solution of tert-butyl 6-oxo-2-azaspiro[3.4]octane-2-carboxylate (5 g, 22.194 mmol, 1 equiv) in THF (100 mL) was added TMSCF₃ (6.92 mL, 44.388 mmol, 2 equiv) and TBAF (1M in THF) (46.61 mL, 46.607 mmol, 2.1 equiv) dropwise at 0° C. under nitrogen atmosphere. The resulting mixture was stirred for 2 h at room temperature under nitrogen atmosphere. The resulting mixture was diluted with water (200 mL) and extracted with EtOAc (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford tert-butyl 6-hydroxy-6-(trifluoromethyl)-2-azaspiro[3.4]octane-2-carboxylate (4.2 g, 64%) as a yellow solid. LC-MS (ES, m/z)=281.15 [M-tBu+ACN+1]⁺.

Step 2. Preparation of 6-(trifluoromethyl)-2-azaspiro[3.4]octan-6-ol. To a stirred solution of tert-butyl 6-hydroxy-6-(trifluoromethyl)-2-azaspiro[3.4]octane-2-carboxylate (4 g, 13.545 mmol, 1 equiv) in DCM (40 mL) was added TFA (8 mL) dropwise at 0° C. under nitrogen atmosphere. The resulting mixture was stirred for 2 h at room temperature under nitrogen atmosphere. The resulting mixture was concentrated under reduced pressure to afford 6-(trifluoromethyl)-2-azaspiro[3.4]octan-6-ol (3.2 g, crude) as a yellow oil. The crude product was used in the next step directly without further purification. LC-MS (ES, m/z)=196.10 [M+1].

Step 3. Preparation of 2-(2,6-bis(benzyloxy)pyridin-3-yl)-5-(6-hydroxy-6-(trifluoromethyl)-2-azaspiro[3.4]octane-2-carbonyl)isoindolin-1-one. To a stirred mixture of 2-[2,6-bis(benzyloxy)pyridin-3-yl]-1-oxo-3H-isoindole-5-carboxylic acid (900 mg, 1.929 mmol, 1 equiv) and 6-(trifluoromethyl)-2-azaspiro[3.4]octan-6-ol (564.85 mg, 2.893 mmol, 1.5 equiv) in ACN (18 mL) were added NMI (475.22 mg, 5.787 mmol, 3 equiv) and TCFH (811.98 mg, 2.893 mmol, 1.5 equiv) in portions at room temperature. The resulting mixture was stirred for overnight at room temperature. The resulting mixture was diluted with water (50 mL) and extracted with EtOAc (3×30 mL). The combined organic layers were washed with brine (2×20 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(6-hydroxy-6-(trifluoromethyl)-2-azaspiro[3.4]octane-2-carbonyl)isoindolin-1-one (1.1 g, 89%) as a white solid. LC-MS (ES, m/z)=644.45 [M+1]⁺.

Step 4. Preparation of 2-(2-(2,6-bis(benzyloxy)pyridin-3-yl)-1-oxoisoindoline-5-carbonyl)-6-(trifluoromethyl)-2-azaspiro[3.4]octan-6-yl methyl oxalate. To a stirred solution of 2-(2,6-bis(benzyloxy)pyridin-3-yl)-5-(6-hydroxy-6-(trifluoromethyl)-2-azaspiro[3.4]octane-2-carbonyl)isoindolin-1-one (500 mg, 0.777 mmol, 1 equiv) and Et₃N (314.43 mg, 3.108 mmol, 4 equiv) in DCM (10 mL) were added DMAP (4.75 mg, 0.039 mmol, 0.05 equiv) and methyl oxalochloridate (190.32 mg, 1.554 mmol, 2 equiv) dropwise at 0° C. under nitrogen atmosphere. The resulting mixture was stirred overnight at room temperature. The reaction was quenched with Water/Ice at 0° C. The resulting mixture was extracted with EtOAc (3×50 mL). The combined organic layers were washed with brine (2×50 mL), dried over anhydrous Na-₂SO₄. After filtration, the filtrate was concentrated under reduced pressure. The residue was purified by reversed-phase flash chromatography to afford 2-(2-(2,6-bis(benzyloxy)pyridin-3-yl)-1-oxoisoindoline-5-carbonyl)-6-(trifluoromethyl)-2-azaspiro[3.4]octan-6-yl methyl oxalate (420 mg, 74%) as a white solid. LC-MS (ES, m/z)=730.35 [M+1]⁺.

Step 5. Preparation of 2-(2,6-bis(benzyloxy)pyridin-3-yl)-5-(6-(trifluoromethyl)-2-azaspiro[3.4]octane-2-carbonyl)isoindolin-1-one. To a stirred solution of 2-(2-(2,6-bis(benzyloxy)pyridin-3-yl)-1-oxoisoindoline-5-carbonyl)-6-(trifluoromethyl)-2-azaspiro[3.4]octan-6-yl methyl oxalate (400 mg, 0.548 mmol, 1 equiv) in Toluene (8 mL) were added AIBN (90.02 mg, 0.548 mmol, 1 equiv) and TBTH (797.76 mg, 2.740 mmol, 5 equiv) in portions at room temperature under nitrogen atmosphere. The resulting mixture was stirred for 1 h at 120° C. The resulting mixture was diluted with water (100 mL) and extracted with EtOAc (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(6-(trifluoromethyl)-2-azaspiro[3.4]octane-2-carbonyl)isoindolin-1-one (240 mg, 70%) as a brown oil. LC-MS (ES, m/z)=628.40 [M+1]⁺.

Step 6. Preparation of 3-(1-oxo-5-(6-(trifluoromethyl)-2-azaspiro[3.4]octane-2-carbonyl)isoindolin-2-yl)piperidine-2,6-dione. To a stirred solution of 2-(2,6-bis(benzyloxy)pyridin-3-yl)-5-(6-(trifluoromethyl)-2-azaspiro[3.4]octane-2-carbonyl)isoindolin-1-one (100 mg, 0.159 mmol, 1 equiv) in THF (2 mL) were added Pd/C (100 mg, Palladium 10% on Carbon (wetted with ca. 55% Water)) at room temperature. The resulting mixture was stirred overnight at room temperature under hydrogen atmosphere. The resulting mixture was filtered through a Celite pad, the filter cake was washed with THF (3×5 mL). The filtrate was concentrated under reduced pressure. The crude product (55 mg) was purified by Prep-HPLC to afford 3-(1-oxo-5-(6-(trifluoromethyl)-2-azaspiro[3.4]octane-2-carbonyl)isoindolin-2-yl)piperidine-2,6-dione (23 mg, 32%) as a white solid. LC-MS (ES, m/z)=450.15 [M+1]⁺. ¹H NMR (400 MHz, DMSO-d₆) δ 11.00 (s, 1H), 7.88-7.71 (m, 3H), 5.14 (dd, J=13.3, 5.1 Hz, 1H), 4.50 (d, J=17.6 Hz, 1H), 4.38 (d, J=17.6 Hz, 1H), 4.24-4.09 (m, 2H), 4.01-3.87 (m, 2H), 2.92 (ddd, J=17.2, 13.5, 5.4 Hz, 2H), 2.61 (ddd, J=17.3, 4.6, 2.3 Hz, 1H), 2.41 (qd, J=13.2, 4.4 Hz, 1H), 2.16 (dd, J=13.4, 9.2 Hz, 1H), 2.03 (ddt, J=12.8, 5.4, 2.7 Hz, 1H), 1.96-1.79 (m, 4H), 1.64 (dd, J=17.8, 6.0 Hz, 1H).

### Example 223

### Synthesis of (2-(2,6-dioxopiperidin-3-yl)benzo[d]oxazol-5-yl)methyl (2-fluoro-4-(trifluoromethoxy)phenyl)carbamate

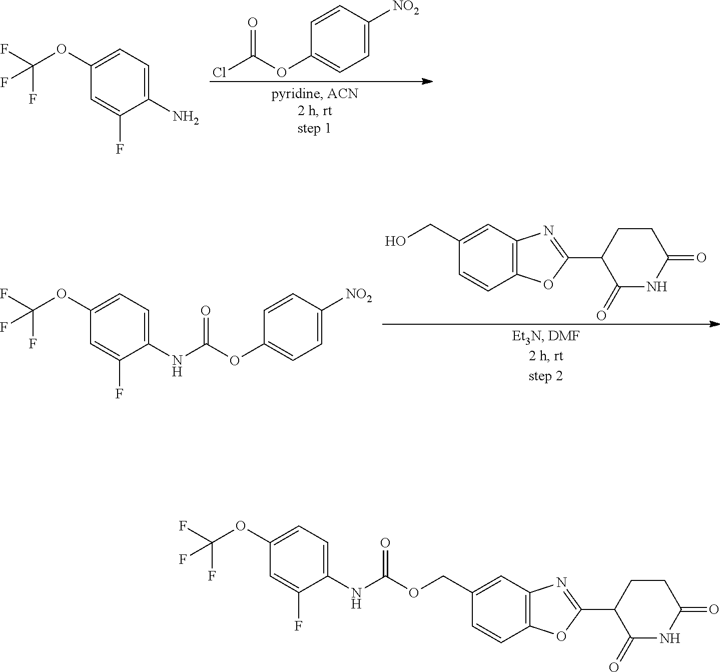 [SMILES: Nc1ccc(OC(F)(F)F)cc1F ; O=C(Cl)Oc1ccc([N+](=O)[O-])cc1 ; O=C(Nc1ccc(OC(F)(F)F)cc1F)Oc1ccc([N+](=O)[O-])cc1 ; O=C1CCC(c2nc3cc(CO)ccc3o2)C(=O)N1 ; O=C1CCC(c2nc3cc(COC(=O)Nc4ccc(OC(F)(F)F)cc4F)ccc3o2)C(=O)N1]

Step 1. Preparation of 4-nitrophenyl (2-fluoro-4-(trifluoromethoxy)phenyl)carbamate. To a stirred mixture of 2-fluoro-4-(trifluoromethoxy)aniline (500 mg, 2.563 mmol, 1 equiv) and Pyridine (608.1 mg, 7.689 mmol, 3 equiv) in ACN (10 mL) was added 4-nitrophenyl carbonochloridate (1.55 g, 7.689 mmol, 3 equiv) at 0° C. under nitrogen atmosphere. The resulting mixture was stirred for 2 h at 0° C. under nitrogen atmosphere. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford 4-nitrophenyl (2-fluoro-4-(trifluoromethoxy)phenyl)carbamate (400 mg, 43%) as a white solid. LC-MS (ES, m/z)=361.25 [M+1]⁺.

Step 2. Preparation of (2-(2,6-dioxopiperidin-3-yl)benzo[d]oxazol-5-yl)methyl (2-fluoro-4-(trifluoromethoxy)phenyl)carbamate. To a stirred mixture of 4-nitrophenyl (2-fluoro-4-(trifluoromethoxy)phenyl)carbamate (100 mg, 0.278 mmol, 1 equiv) and 3-[5-(hydroxymethyl)-1,3-benzoxazol-2-yl]piperidine-2,6-dione (72.2 mg, 0.278 mmol, 1 equiv) in DMF (1 mL) were added Et₃N (84.2 mg, 0.834 mmol, 3 equiv) at room temperature. The resulting mixture was stirred for 2 h at room temperature. The resulting mixture was purified by reversed-phase flash chromatography to afford (2-(2,6-dioxopiperidin-3-yl)benzo[d]oxazol-5-yl)methyl (2-fluoro-4-(trifluoromethoxy)phenyl)carbamate (15 mg, 11%) as a white solid. LC-MS (ES, m/z)=480.05 [M+1]⁺. ¹H NMR (400 MHz, DMSO-d₆) δ 11.13 (s, 1H), 9.67 (s, 1H), 7.85-7.73 (m, 3H), 7.53-7.40 (m, 2H), 7.26-7.19 (m, 1H), 5.29 (s, 2H), 4.52 (dd, J=11.3, 5.0 Hz, 1H), 2.76-2.59 (m, 2H), 2.41 (td, J=11.4, 4.1 Hz, 1H), 2.30 (dt, J=13.1, 5.0 Hz, 1H).

### Example 224

### Synthesis of (2-(2,6-dioxopiperidin-3-yl)benzo[d]oxazol-5-yl)methyl (5-chloro-2-fluorophenyl)carbamate

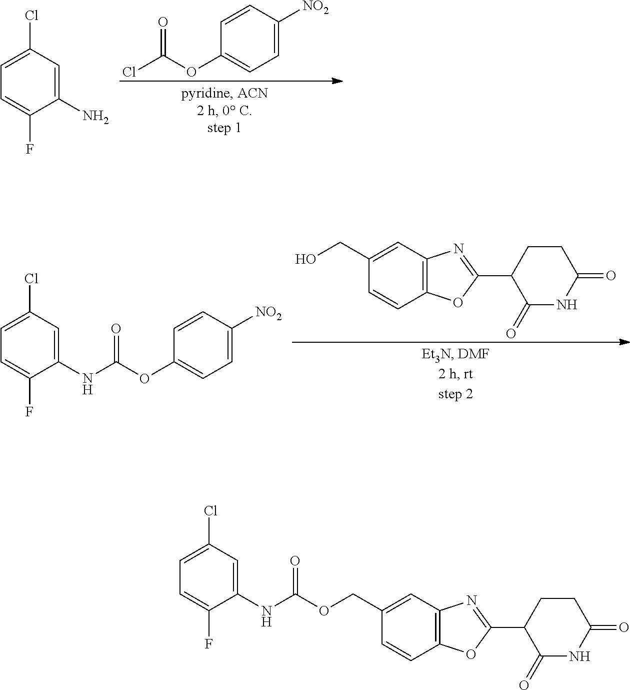 [SMILES: Nc1cc(Cl)ccc1F ; O=C(Cl)Oc1ccc([N+](=O)[O-])cc1 ; O=C(Nc1cc(Cl)ccc1F)Oc1ccc([N+](=O)[O-])cc1 ; O=C1CCC(c2nc3cc(CO)ccc3o2)C(=O)N1 ; O=C1CCC(c2nc3cc(COC(=O)Nc4cc(Cl)ccc4F)ccc3o2)C(=O)N1]

Step 1. Preparation of 4-nitrophenyl (5-chloro-2-fluorophenyl)carbamate. To a stirred mixture of 5-chloro-2-fluoroaniline (50 mg, 0.344 mmol, 1 equiv) and Pyridine (81.5 mg, 1.032 mmol, 3 equiv) in ACN (1 mL) were added 4-nitrophenyl carbonochloridate (207.7 mg, 1.032 mmol, 3 equiv) in portions at 0° C. under nitrogen atmosphere. The resulting mixture was stirred for 2 h at 0° C. under nitrogen atmosphere. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to afford 4-nitrophenyl N-(5-chloro-2-fluorophenyl)carbamate (100 mg, 94%) as a white solid. LC-MS (ES, m/z)=no mass signal.

Step 2. Preparation of (2-(2,6-dioxopiperidin-3-yl)benzo[d]oxazol-5-yl)methyl (5-chloro-2-fluorophenyl)carbamate. To a stirred mixture of 4-nitrophenyl N-(5-chloro-2-fluorophenyl)carbamate (100 mg, 0.322 mmol, 1 equiv) and 3-[5-(hydroxymethyl)-1,3-benzoxazol-2-yl]piperidine-2,6-dione (83.8 mg, 0.322 mmol, 1 equiv) in DMF (2 mL) were added Et₃N (97.7 mg, 0.966 mmol, 3 equiv) at room temperature. The resulting mixture was stirred for 2 h at room temperature. The resulting mixture was diluted with water (30 mL) and extracted with EtOAc (3×20 mL). The combined organic layers were washed with brine (2×20 mL), dried over anhydrous Na₂SO₄. After filtration, the filtrate was concentrated under reduced pressure. The resulting mixture was purified by reversed-phase flash chromatography to afford (2-(2,6-dioxopiperidin-3-yl)benzo[d]oxazol-5-yl)methyl (5-chloro-2-fluorophenyl)carbamate (16 mg, 12%) as a white solid. LC-MS (ES, m/z)=432.05 [M+1]⁺. ¹H NMR (400 MHz, DMSO-d₆) δ 11.13 (s, 1H), 9.75 (s, 1H), 7.88-7.73 (m, 3H), 7.50 (dd, J=8.5, 1.7 Hz, 1H), 7.29 (dd, J=10.6, 8.8 Hz, 1H), 7.18 (ddd, J=8.8, 4.2, 2.7 Hz, 1H), 5.29 (s, 2H), 4.52 (dd, J=11.3, 5.1 Hz, 1H), 2.77-2.58 (m, 2H), 2.46-2.27 (m, 2H).

### Example 225

### Synthesis of 2-(2,6-dioxopiperidin-3-yl)-N-(6-(4-fluorophenyl)-6-hydroxyspiro[3.3]heptan-2-yl)-1-oxoisoindoline-5-carboxamide

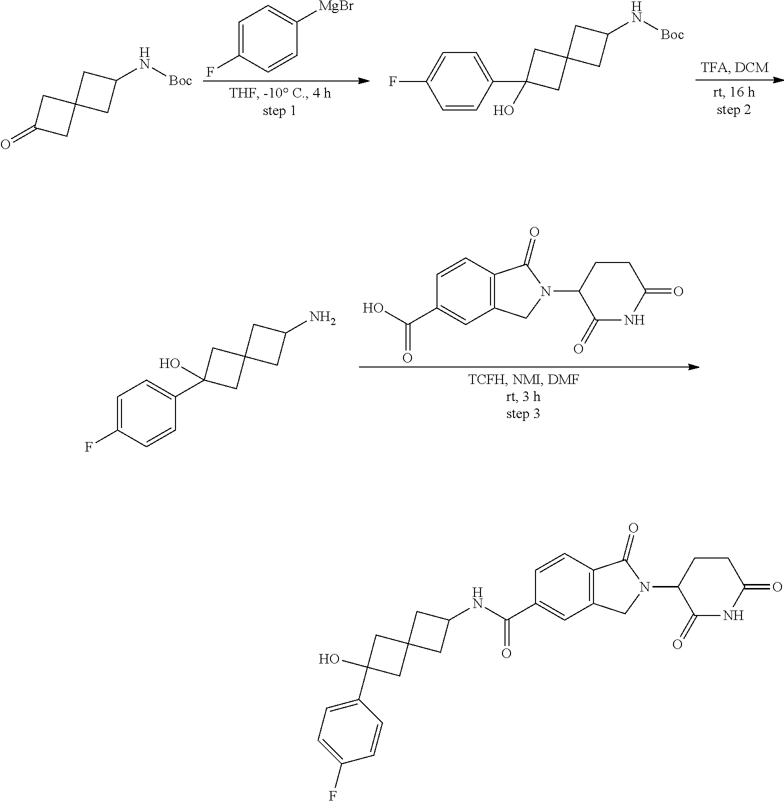

Step 1. Preparation of tert-butyl (6-(4-fluorophenyl)-6-hydroxyspiro[3.3]heptan-2-yl)carbamate. To a stirred solution of tert-butyl N-{6-oxospiro[3.3]heptan-2-yl}carbamate (1.0 g, 4.439 mmol, 1 equiv) in tetrahydrofuran (20 mL) were added bromo(4-fluorophenyl)magnesium (5.33 mL, 5.327 mmol, 1.2 equiv) dropwise at −10° C. under nitrogen atmosphere. The resulting mixture was stirred for 4 h at −10° C. under nitrogen atmosphere. The reaction was quenched with Water/Ice at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl (6-(4-fluorophenyl)-6-hydroxyspiro[3.3]heptan-2-yl)carbamate (820 mg, 58%) as a white solid. LC-MS (ES, m/z)=no mass signal.

Step 2. Preparation of 6-amino-2-(4-fluorophenyl)spiro[3.3]heptan-2-ol. To a stirred solution of tert-butyl (6-(4-fluorophenyl)-6-hydroxyspiro[3.3]heptan-2-yl)carbamate (150 mg, 0.467 mmol, 1 equiv) in DCM (3 mL) was added TFA (1 mL) dropwise at 0° C. The resulting mixture was stirred overnight at room temperature. The resulting mixture was concentrated under reduced pressure to afford 6-amino-2-(4-fluorophenyl)spiro[3.3]heptan-2-ol (80 mg, crude) as a yellow oil. The crude product was used in the next step directly without further purification. LC-MS (ES, m/z)=222.30 [M+1]⁺.

Step 3. Preparation of 2-(2,6-dioxopiperidin-3-yl)-N-(6-(4-fluorophenyl)-6-hydroxyspiro[3.3]heptan-2-yl)-1-oxoisoindoline-5-carboxamide. To a stirred solution of 2-(2,6-dioxopiperidin-3-yl)-1-oxo-3H-isoindole-5-carboxylic acid (50 mg, 0.173 mmol, 1 equiv) and 6-amino-2-(4-fluorophenyl)spiro[3.3]heptan-2-ol (76.76 mg, 0.346 mmol, 2 equiv) in DMF (1 mL) were added NMI (42.73 mg, 0.519 mmol, 3 equiv) and TCFH (73.00 mg, 0.259 mmol, 1.5 equiv) in portions at room temperature. The resulting mixture was stirred for 3 h at room temperature. The resulting mixture was diluted with water (50 mL) and extracted with EtOAc (3×20 mL). The combined organic layers were washed with brine (2×30 mL), dried over anhydrous Na₂SO₄. After filtration, the filtrate was concentrated under reduced pressure. The crude product (50 mg) was purified by Prep-HPLC to afford 2-(2,6-dioxopiperidin-3-yl)-N-(6-(4-fluorophenyl)-6-hydroxyspiro[3.3]heptan-2-yl)-1-oxoisoindoline-5-carboxamide (7.7 mg, 9%) as a white solid. LC-MS (ES, m/z)=492.15 [M+1]⁺. ¹H NMR (400 MHz, DMSO-d₆) δ 11.00 (s, 1H), 8.75 (d, J=7.3 Hz, 1H), 8.06-7.93 (m, 2H), 7.79 (d, J=7.9 Hz, 1H), 7.50-7.40 (m, 2H), 7.18-7.08 (m, 2H), 5.14 (dd, J=13.3, 5.1 Hz, 1H), 4.51 (d, J=17.6 Hz, 1H), 4.43-4.25 (m, 2H), 2.92 (ddd, J=17.2, 13.6, 5.4 Hz, 1H), 2.60 (ddd, J=11.6, 8.7, 3.4 Hz, 2H), 2.49-2.39 (m, 4H), 2.34-2.21 (m, 3H), 2.09-1.99 (m, 2H).

### Example 226

### Synthesis of 2-(2,6-dioxopiperidin-3-yl)-1-oxo-N-(6-(4-(trifluoromethyl)phenyl)spiro[3.3]heptan-2-yl)isoindoline-5-carboxamide

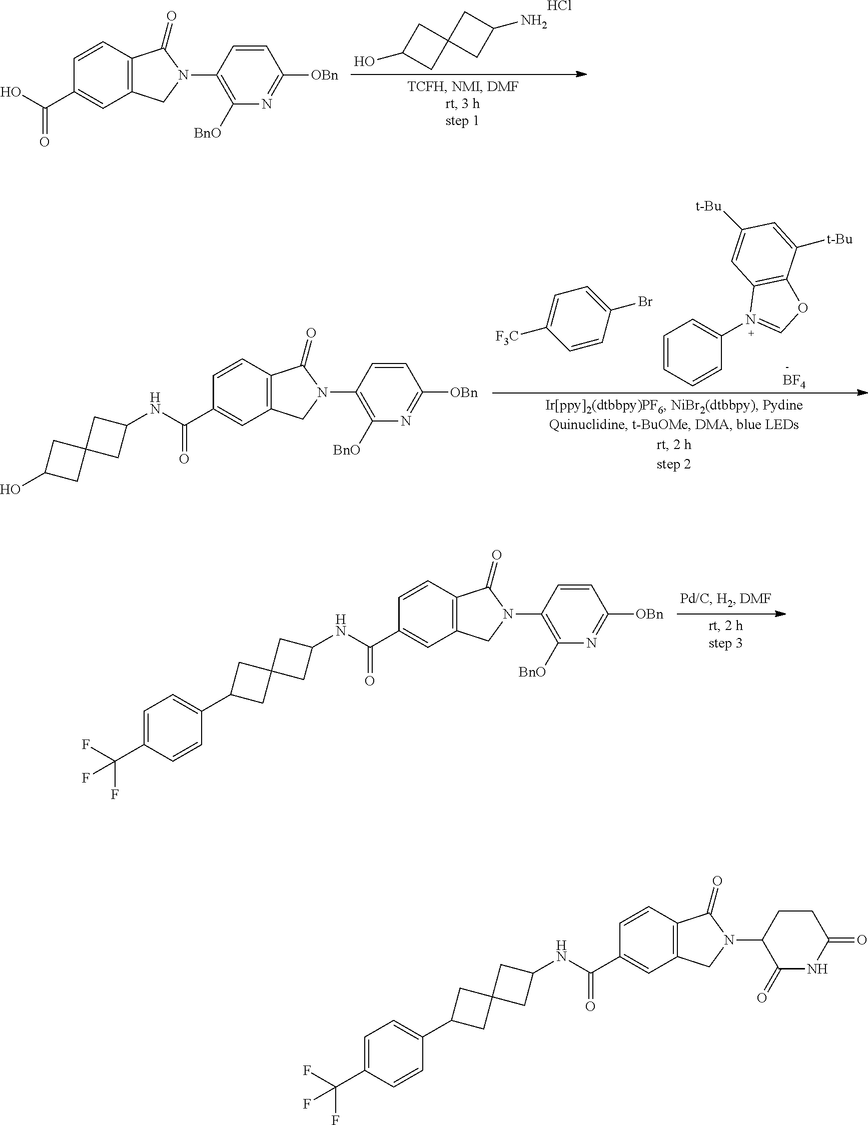

Step 1. Preparation of 2-(2,6-bis(benzyloxy)pyridin-3-yl)-N-(6-hydroxyspiro[3.3]heptan-2-yl)-1-oxoisoindoline-5-carboxamide. To a stirred mixture of 2-[2,6-bis(benzyloxy)pyridin-3-yl]-1-oxo-3H-isoindole-5-carboxylic acid (1 g, 2.144 mmol, 1.00 equiv) and 6-aminospiro[3.3]heptan-2-ol hydrochloride (0.35 g, 2.144 mmol, 1.00 equiv) in DMF (15 mL) were added NMI (0.7 g, 8.576 mmol, 4.00 equiv) and TCFH (0.9 g, 3.216 mmol, 1.50 equiv) in portions at room temperature under air atmosphere. The resulting mixture was stirred for 2 h at room temperature under nitrogen atmosphere. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-N-(6-hydroxyspiro[3.3]heptan-2-yl)-1-oxoisoindoline-5-carboxamide (1 g, 81%) as a yellow solid. LC-MS (ES, m/z)=576.35 [M+1]⁺.

Step 2. Preparation of 2-(2,6-bis(benzyloxy)pyridin-3-yl)-1-oxo-N-(6-(4-(trifluoromethyl)phenyl)spiro[3.3]heptan-2-yl)isoindoline-5-carboxamide. To a stirred mixture of 2-(2,6-bis(benzyloxy)pyridin-3-yl)-N-(6-hydroxyspiro[3.3]heptan-2-yl)-1-oxoisoindoline-5-carboxamide (150 mg, 0.261 mmol, 1 equiv) and 5,7-di-tert-butyl-3-phenylbenzo[d]oxazol-3-ium tetrafluoroborate (164.8 mg, 0.417 mmol, 1.60 equiv) in 2-methoxy-2-methylpropane (3 mL) was added Pyridine (33 mg, 0.417 mmol, 1.60 equiv) dropwise at 0° C. under nitrogen atmosphere. The resulting mixture was stirred for 10 min at 0° C. under nitrogen atmosphere. Then, To the above mixture was added a mixture of 1-bromo-4-(trifluoromethyl)benzene (88 mg, 0.391 mmol, 1.50 equiv), [Ir(dtbbpy)(ppy)2] [PF₆] (2.4 mg, 0.003 mmol, 0.01 equiv), NiBr₂(dtbbpy) (6.4 mg, 0.013 mmol, 0.05 equiv) and 1-azabicyclo[2.2.2]octane (50.7 mg, 0.456 mmol, 1.75 equiv) in DMA (3 mL) at room temperature under nitrogen atmosphere. The resulting mixture was stirred for 2 h at room temperature under blue LEDs atmosphere. The resulting mixture was diluted with water (50 mL) and extracted with EtOAc (3×30 mL). The combined organic layers were washed with brine (2×30 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-1-oxo-N-(6-(4-(trifluoromethyl)phenyl)spiro[3.3]heptan-2-yl)isoindoline-5-carboxamide (90 mg, 49%) as a yellow solid. LC-MS (ES, m/z)=704.45 [M+1]⁺.

Step 3. Preparation of 2-(2,6-dioxopiperidin-3-yl)-1-oxo-N-(6-(4-(trifluoromethyl)phenyl)spiro[3.3]heptan-2-yl)isoindoline-5-carboxamide. To a solution of 2-(2,6-bis(benzyloxy)pyridin-3-yl)-1-oxo-N-(6-(4-(trifluoromethyl)phenyl)spiro[3.3]heptan-2-yl)isoindoline-5-carboxamide (90 mg, 0.128 mmol, 1 equiv) in DMF (4 mL) was added Pd/C (45 mg, Palladium 10% on Carbon (wetted with ca. 55% Water)) under nitrogen atmosphere in a 25 mL round-bottom flask. The mixture was hydrogenated at room temperature for 2 h under hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford 2-(2,6-dioxopiperidin-3-yl)-1-oxo-N-(6-(4-(trifluoromethyl)phenyl)spiro[3.3]heptan-2-yl)isoindoline-5-carboxamide (63 mg, 94%) as an off-white solid. LC-MS (ES, m/z)=526.10 [M+1]⁺. ¹H NMR (400 MHz, DMSO-d₆) δ 11.01 (s, 1H), 8.79 (d, J=7.4 Hz, 1H), 8.06 (t, J=1.1 Hz, 1H), 7.98 (dd, J=8.0, 1.5 Hz, 1H), 7.84-7.78 (m, 1H), 7.65 (d, J=8.1 Hz, 2H), 7.44 (d, J=8.0 Hz, 2H), 5.15 (dd, J=13.3, 5.1 Hz, 1H), 4.52 (d, J=17.6 Hz, 1H), 4.45-4.31 (m, 2H), 3.53 (p, J=8.9 Hz, 1H), 2.93 (ddd, J=17.2, 13.6, 5.4 Hz, 1H), 2.59 (dddd, J=24.0, 14.2, 5.1, 2.9 Hz, 3H), 2.49-2.33 (m, 2H), 2.32-1.99 (m, 6H).

### Example 227

### Synthesis of 2-(2,6-dioxopiperidin-3-yl)-1-oxo-N-(6-(p-tolyl spiro[3.3]heptan-2-yl)isoindoline-5-carboxamide

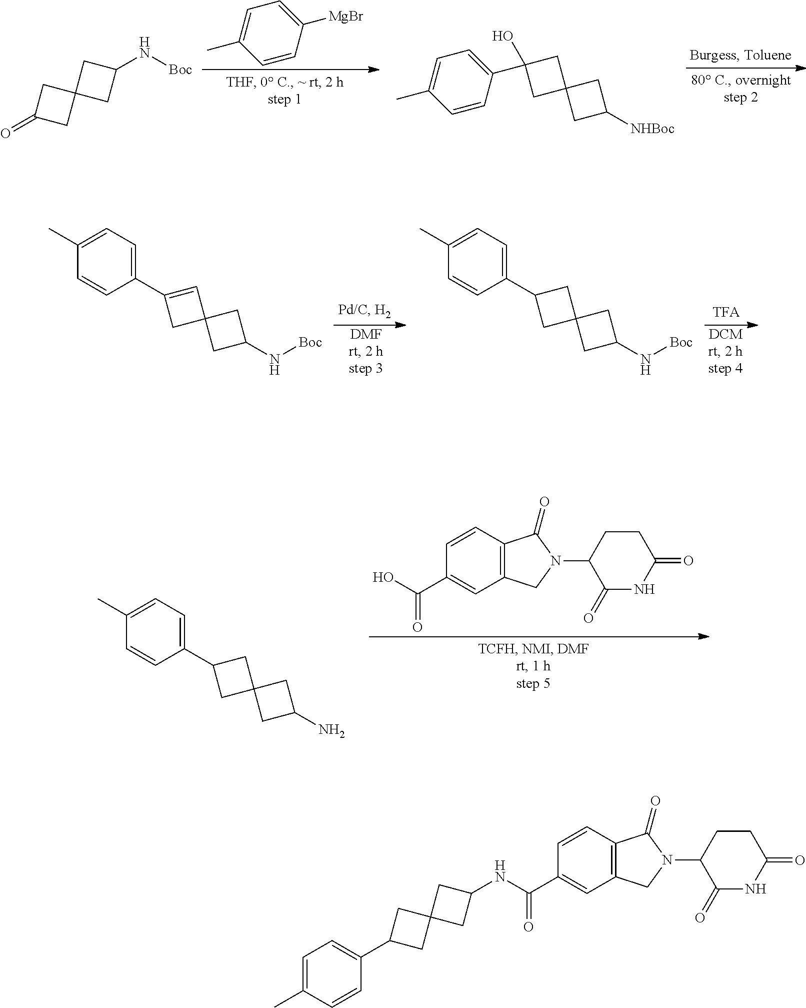

Step 1. Preparation of tert-butyl (6-hydroxy-6-(p-tolyl)spiro[3.3]heptan-2-yl)carbamate. To a stirred mixture of tert-butyl N-{6-oxospiro[3.3]heptan-2-yl}carbamate (500 mg, 2.219 mmol, 1 equiv) in THF (10 mL) was added bromo(4-methylphenyl)magnesium (5.55 mL, 5.547 mmol, 2.5 equiv) (1M in THF) dropwise at 0° C. under nitrogen atmosphere. The resulting mixture was stirred for an additional 2 h at room temperature. The reaction was quenched by the addition of sat. NH₄Cl (aq.) (10 mL) at 0° C. The resulting mixture was concentrated under vacuum. The resulting mixture was diluted with water (100 mL). The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl (6-hydroxy-6-(p-tolyl)spiro[3.3]heptan-2-yl)carbamate (600 mg, 85%) as an off-white solid. ¹H NMR (400 MHz, DMSO-d₆) δ 7.30-7.24 (m, 2H), 7.11 (d, J=7.9 Hz, 2H), 7.01 (d, J=7.9 Hz, 1H), 5.23 (s, 1H), 3.78 (h, J=8.2 Hz, 1H), 2.47 (d, J=2.9 Hz, 1H), 2.43-2.28 (m, 3H), 2.27 (s, 3H), 2.21 (dd, J=12.2, 2.5 Hz, 1H), 2.12-2.03 (m, 1H), 1.98 (dd, J=10.9, 8.8 Hz, 1H), 1.81 (dd, J=10.8, 8.8 Hz, 1H), 1.35 (s, 9H).

Step 2. Preparation of tert-butyl (6-(p-tolyl)spiro[3.3]hept-5-en-2-yl)carbamate. A mixture of tert-butyl (6-hydroxy-6-(p-tolyl)spiro[3.3]heptan-2-yl)carbamate (600 mg, 1.890 mmol, 1 equiv) and Burgess reagent (1.13 g, 4.725 mmol, 2.5 equiv) in Toluene (12 mL) was stirred for overnight at 80° C. under nitrogen atmosphere. The mixture was allowed to cool down to room temperature. The resulting mixture was concentrated under vacuum. The residue was purified by reversed-phase flash chromatography to afford tert-butyl (6-(p-tolyl)spiro[3.3]hept-5-en-2-yl)carbamate (300 mg, 53%) as an off-white solid. LC-MS (ES, m/z)=285.10 [M-tBu+ACN+1]⁺.

Step 3. Preparation of tert-butyl (6-(p-tolyl)spiro[3.3]heptan-2-yl)carbamate. To a solution of tert-butyl (6-(p-tolyl)spiro[3.3]hept-5-en-2-yl)carbamate (50 mg, 0.167 mmol, 1 equiv) in DMF (1 mL) was added Pd/C (25 mg, Palladium 10% on Carbon (wetted with ca. 55% Water)) under nitrogen atmosphere. The mixture was hydrogenated at room temperature for 2 h under the hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford tert-butyl (6-(p-tolyl)spiro[3.3]heptan-2-yl)carbamate (50 mg, 99%) as an off-white solid. LC-MS (ES, m/z)=287.15 [M-tBu+ACN+1]⁺.

Step 4. Preparation of 6-(p-tolyl)spiro[3.3]heptan-2-amine. To a stirred solution of tert-butyl (6-(p-tolyl)spiro[3.3]heptan-2-yl)carbamate (50 mg, 0.166 mmol, 1 equiv) in DCM (1 mL) was added TFA (0.2 mL, 2.693 mmol, 16.23 equiv) dropwise at 0° C. under nitrogen atmosphere. The resulting mixture was stirred for 2 h at room temperature under nitrogen atmosphere. The resulting mixture was concentrated under vacuum to afford 6-(p-tolyl)spiro[3.3]heptan-2-amine (40 mg, crude) as a brown oil. The crude product was used in the next step directly without further purification. LC-MS (ES, m/z)=202.10 [M+1]⁺.

Step 5. Preparation of 2-(2,6-dioxopiperidin-3-yl)-1-oxo-N-(6-(p-tolyl)spiro[3.3]heptan-2-yl)isoindoline-5-carboxamide. To a stirred mixture of 2-(2,6-dioxopiperidin-3-yl)-1-oxo-3H-isoindole-5-carboxylic acid (40 mg, 0.139 mmol, 1 equiv) and 6-(4-methylphenyl)spiro[3.3]heptan-2-amine (41.9 mg, 0.209 mmol, 1.5 equiv) in DMF (1 mL) were added NMI (57 mg, 0.694 mmol, 5 equiv) and TCFH (58.4 mg, 0.208 mmol, 1.5 equiv) at room temperature under air atmosphere. The resulting mixture was stirred for 1 h at room temperature under nitrogen atmosphere. The resulting mixture was diluted with water (20 mL) and extracted with EtOAc (3×10 mL). The combined organic layers were washed with brine (2×20 mL) and concentrated under reduced pressure. The residue was purified by reversed-phase flash chromatography to afford 2-(2,6-dioxopiperidin-3-yl)-1-oxo-N-(6-(p-tolyl)spiro[3.3]heptan-2-yl)isoindoline-5-carboxamide (22 mg, 33%) as an off-white solid. LC-MS (ES, m/z)=472.10 [M+1]⁺. ¹H NMR (400 MHz, DMSO-d₆) δ 11.01 (s, 1H), 8.78 (d, J=7.4 Hz, 1H), 8.06 (dd, J=1.5, 0.8 Hz, 1H), 7.97 (dd, J=8.0, 1.5 Hz, 1H), 7.80 (dd, J=7.9, 0.7 Hz, 1H), 7.09 (s, 4H), 5.14 (dd, J=13.3, 5.1 Hz, 1H), 4.51 (d, J=17.5 Hz, 1H), 4.44-4.29 (m, 2H), 3.36 (d, J=17.8 Hz, 1H), 2.92 (ddd, J=17.3, 13.7, 5.5 Hz, 1H), 2.66-2.52 (m, 2H), 2.49-2.36 (m, 2H), 2.33-2.17 (m, 6H), 2.15-1.98 (m, 4H), 1.23 (s, 1H).

### Example 228

### Synthesis of 3-(5-(6-(3,4-difluorophenyl)-2-azaspiro[3.3]heptane-2-carbonyl)-1-oxoisoindolin-2-yl)piperidine-2,6-dione

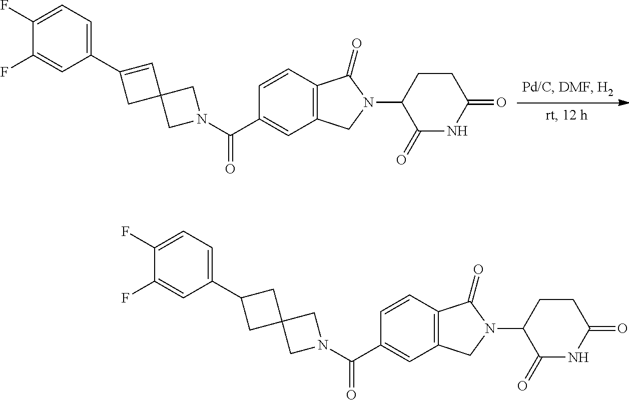 [SMILES: O=C1CCC(N2Cc3cc(C(=O)N4CC5(C=C(c6ccc(F)c(F)c6)C5)C4)ccc3C2=O)C(=O)N1 ; O=C1CCC(N2Cc3cc(C(=O)N4CC5(CC(c6ccc(F)c(F)c6)C5)C4)ccc3C2=O)C(=O)N1]

Step 1. Preparation of 3-(5-(6-(3,4-difluorophenyl)-2-azaspiro[3.3]heptane-2-carbonyl)-1-oxoisoindolin-2-yl)piperidine-2,6-dione. A mixture of 3-{5-[6-(3,4-difluorophenyl)-2-azaspiro[3.3]hept-5-ene-2-carbonyl]-1-oxo-3H-isoindol-2-yl}piperidine-2,6-dione (150 mg, 0.314 mmol, 1 equiv) in DMF (2 mL) was added Pd/C (100 mg, Palladium 10% on Carbon (wetted with ca. 55% Water)) at room temperature. The resulting mixture was stirred for 2 h at room temperature under hydrogen atmosphere. The resulting mixture was filtered through a Celite pad, the filter cake was washed with THF (3×5 mL). The filtrate was concentrated under reduced pressure. The residue was purified by reversed-phase flash chromatography to afford 3-(5-(6-(3,4-difluorophenyl)-2-azaspiro[3.3]heptane-2-carbonyl)-1-oxoisoindolin-2-yl)piperidine-2,6-dione (90 mg, 59%) as an off-white solid. LC-MS (ES, m/z)=480.20 [M+1]⁺. ¹H NMR (400 MHz, DMSO-d₆) δ 11.01 (s, 1H), 7.86 (d, J=9.6 Hz, 1H), 7.83-7.68 (m, 2H), 7.33 (q, J=9.0 Hz, 2H), 7.05 (dd, J=8.7, 4.7 Hz, 1H), 5.14 (ddd, J=13.4, 5.2, 2.3 Hz, 1H), 4.57-4.32 (m, 3H), 4.23 (d, J=6.5 Hz, 2H), 4.01 (s, 1H), 3.41 (dd, J=19.7, 11.0 Hz, 1H), 2.92 (ddd, J=18.2, 13.5, 5.4 Hz, 1H), 2.70-2.53 (m, 3H), 2.40 (dd, J=15.1, 11.0 Hz, 1H), 2.29 (qd, J=11.9, 10.6, 5.8 Hz, 2H), 2.04 (q, J=9.7, 7.6 Hz, 1H).

### Example 229

### Synthesis of 3-(5-(6-(2,4-difluorophenyl)-2-azaspiro[3.3]heptane-2-carbonyl)-1-oxoisoindolin-2-yl)piperidine-2,6-dione

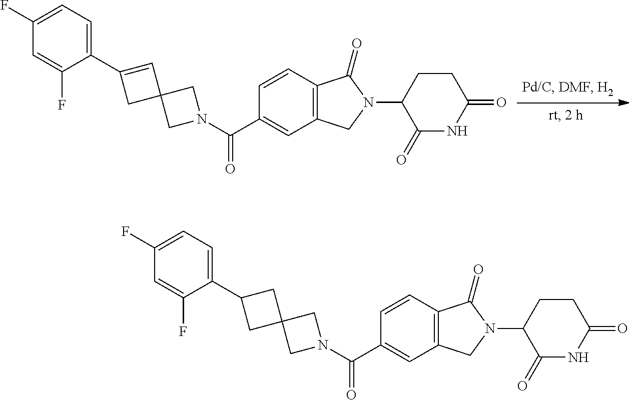 [SMILES: O=C1CCC(N2Cc3cc(C(=O)N4CC5(C=C(c6ccc(F)cc6F)C5)C4)ccc3C2=O)C(=O)N1 ; O=C1CCC(N2Cc3cc(C(=O)N4CC5(CC(c6ccc(F)cc6F)C5)C4)ccc3C2=O)C(=O)N1]

Step 1. Preparation of 3-(5-(6-(2,4-difluorophenyl)-2-azaspiro[3.3]heptane-2-carbonyl)-1-oxoisoindolin-2-yl)piperidine-2,6-dione. A mixture of 3-{5-[6-(2,4-difluorophenyl)-2-azaspiro[3.3]hept-5-ene-2-carbonyl]-1-oxo-3H-isoindol-2-yl}piperidine-2,6-dione (25 mg, 0.052 mmol, 1 equiv) in DMF (0.5 mL) was added Pd/C (15 mg, Palladium 10% on Carbon (wetted with ca. 55% Water)) at room temperature. The resulting mixture was stirred for 2 h at room temperature under hydrogen atmosphere. The resulting mixture was filtered through a Celite pad, the filter cake was washed with THF (3×5 mL). The filtrate was concentrated under reduced pressure. The residue was purified by reversed-phase flash chromatography to afford 3-(5-(6-(2,4-difluorophenyl)-2-azaspiro[3.3]heptane-2-carbonyl)-1-oxoisoindolin-2-yl)piperidine-2,6-dione (16 mg, 61%) as an off-white solid. LC-MS (ES, m/z)=480.15 [M+1]⁺. ¹H NMR (400 MHz, DMSO-d₆) δ 11.01 (s, 1H), 7.86 (d, J=11.5 Hz, 1H), 7.77 (p, J=7.8 Hz, 2H), 7.36 (dq, J=15.5, 8.2 Hz, 1H), 7.15 (ddd, J=10.6, 9.3, 2.6 Hz, 1H), 7.04 (p, J=7.0 Hz, 1H), 5.14 (dt, J=13.2, 4.4 Hz, 1H), 4.63-4.33 (m, 3H), 4.24 (s, 2H), 4.01 (s, 1H), 3.50 (dq, J=27.0, 9.0 Hz, 1H), 3.01-2.83 (m, 1H), 2.70-2.54 (m, 3H), 2.35 (dq, J=22.2, 12.3, 11.5 Hz, 3H), 2.04 (dd, J=16.8, 9.0 Hz, 1H).

### Example 230

### Synthesis of 3-(1-oxo-5-6-(4-(trifluoromethyl)phenyl)-2-azaspiro[3.3]heptane-2-carbonyl)isoindolin-2-yl)piperidine-2,6-dione

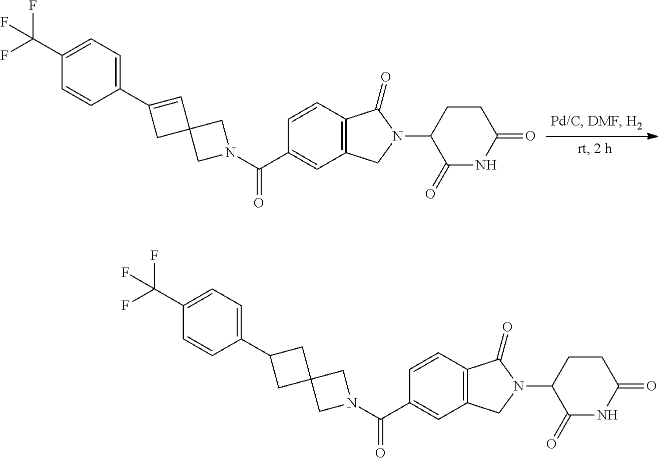 [SMILES: O=C1CCC(N2Cc3cc(C(=O)N4CC5(C=C(c6ccc(C(F)(F)F)cc6)C5)C4)ccc3C2=O)C(=O)N1 ; O=C1CCC(N2Cc3cc(C(=O)N4CC5(CC(c6ccc(C(F)(F)F)cc6)C5)C4)ccc3C2=O)C(=O)N1]

Step 1. Preparation of 3-(1-oxo-5-(6-(4-(trifluoromethyl)phenyl)-2-azaspiro[3.3]heptane-2-carbonyl)isoindolin-2-yl)piperidine-2,6-dione. A mixture of 3-(1-oxo-5-{6-[4-(trifluoromethyl)phenyl]-2-azaspiro[3.3]hept-5-ene-2-carbonyl}-3H-isoindol-2-yl)piperidine-2,6-dione (130 mg, 0.255 mmol, 1 equiv) in DMF (2 mL) was added Pd/C (100 mg, Palladium 10% on Carbon (wetted with ca. 55% Water)) at room temperature. The resulting mixture was stirred for 2 h at room temperature under hydrogen atmosphere. The resulting mixture was filtered through a Celite pad, the filter cake was washed with THF (3×5 mL). The filtrate was concentrated under reduced pressure. The residue was purified by Prep-HPLC to afford 3-(1-oxo-5-(6-(4-(trifluoromethyl)phenyl)-2-azaspiro[3.3]heptane-2-carbonyl)isoindolin-2-yl)piperidine-2,6-dione (11.4 mg, 8.63%) as an off-white solid. LC-MS (ES, m/z)=512.10 [M+1]⁺. ¹H NMR (400 MHz, DMSO-d₆) δ 11.01 (s, 1H), 7.87 (d, J=11.4 Hz, 1H), 7.77 (p, J=7.9 Hz, 2H), 7.65 (d, J=7.9 Hz, 2H), 7.44 (dd, J=8.4, 3.2 Hz, 2H), 5.15 (dt, J=13.3, 4.4 Hz, 1H), 4.57-4.33 (m, 3H), 4.25 (s, 2H), 4.03 (s, 1H), 3.49 (dt, J=30.4, 8.7 Hz, 1H), 3.01-2.85 (m, 1H), 2.72-2.56 (m, 3H), 2.46-2.25 (m, 3H), 2.03 (d, J=12.4 Hz, 1H).

### Example 231

### Synthesis of 3-(1-oxo-5-(6-(p-tolyl)-2-azaspiro[3.3]heptane-2-carbonyl)isoindolin-2-yl)piperidine-2,6-dione

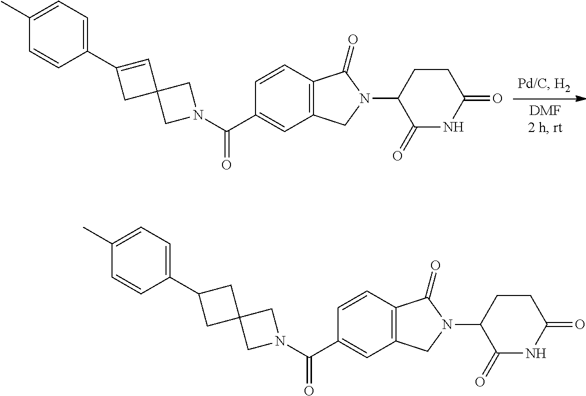 [SMILES: Cc1ccc(C2=CC3(C2)CN(C(=O)c2ccc4c(c2)CN(C2CCC(=O)NC2=O)C4=O)C3)cc1 ; Cc1ccc(C2CC3(C2)CN(C(=O)c2ccc4c(c2)CN(C2CCC(=O)NC2=O)C4=O)C3)cc1]

Step 1. Preparation of 3-(1-oxo-5-(6-(p-tolyl)-2-azaspiro[3.3]heptane-2-carbonyl)isoindolin-2-yl)piperidine-2,6-dione. To a solution of 3-{5-[6-(4-methylphenyl)-2-azaspiro[3.3]hept-5-ene-2-carbonyl]-1-oxo-3H-isoindol-2-yl}piperidine-2,6-dione (25 mg, 0.055 mmol, 1 equiv) in dimethylformamide (4 mL) was added Pd/C (10 mg, Palladium 10% on Carbon (wetted with ca. 55% Water)) under nitrogen atmosphere in a 8 mL vial. The mixture was hydrogenated at room temperature for 2 h under hydrogen atmosphere using a hydrogen balloon, The resulting mixture was filtered through a Celite pad, and the filter cake was washed with THF (3×5 mL). The filtrate was concentrated under reduced pressure. The residue was purified by reversed-phase flash chromatography to afford 3-(1-oxo-5-(6-(p-tolyl)-2-azaspiro[3.3]heptane-2-carbonyl)isoindolin-2-yl)piperidine-2,6-dione (18 mg, 70%) as an off-white solid. LC-MS (ES, m/z)=458.20 [M+1]⁺. ¹H NMR (400 MHz, DMSO-d₆) δ 11.01 (s, 1H), 7.86 (d, J=10.4 Hz, 1H), 7.77 (p, J=8.0 Hz, 2H), 7.09 (d, J=3.0 Hz, 4H), 5.14 (dt, J=13.0, 4.4 Hz, 1H), 4.56-4.33 (m, 3H), 4.23 (d, J=4.4 Hz, 2H), 4.01 (s, 1H), 2.99-2.85 (m, 1H), 2.65-2.52 (m, 3H), 2.42 (d, J=13.1 Hz, 1H), 2.31-2.18 (m, 5H), 2.02 (dd, J=14.1, 6.8 Hz, 1H), 1.24 (d, J=2.8 Hz, 1H).

### Example 232

### Synthesis of 4-(2-(2-(2,6-dioxopiperidin-3-yl)-1-oxoisoindoline-5-carbonyl azaspiro[3.3]heptan-6-yl)benzonitrile

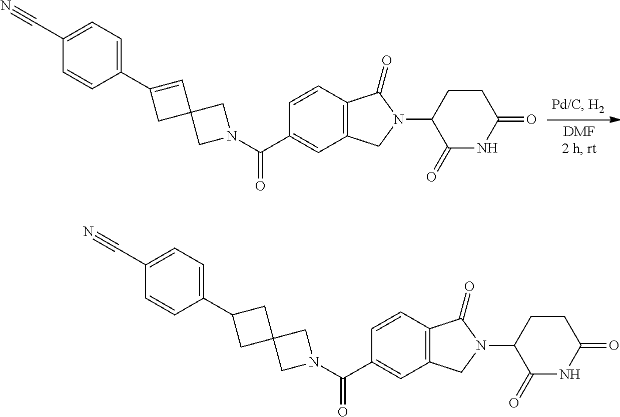 [SMILES: N#Cc1ccc(C2=CC3(C2)CN(C(=O)c2ccc4c(c2)CN(C2CCC(=O)NC2=O)C4=O)C3)cc1 ; N#Cc1ccc(C2CC3(C2)CN(C(=O)c2ccc4c(c2)CN(C2CCC(=O)NC2=O)C4=O)C3)cc1]

Step 1. Preparation of 4-(2-(2-(2,6-dioxopiperidin-3-yl)-1-oxoisoindoline-5-carbonyl)-2-azaspiro[3.3]heptan-6-yl)benzonitrile. To a solution of 3-{5-[6-(4-methylphenyl)-2-azaspiro[3.3]hept-5-ene-2-carbonyl]-1-oxo-3H-isoindol-2-yl}piperidine-2,6-dione (35 mg, 0.077 mmol, 1 equiv) in DMF (1 mL) was added Pd/C (15 mg, Palladium 10% on Carbon (wetted with ca. 55% Water)) under nitrogen atmosphere in a 8 mL vial. The mixture was hydrogenated at room temperature for 2 h under hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford 4-(2-(2-(2,6-dioxopiperidin-3-yl)-1-oxoisoindoline-5-carbonyl)-2-azaspiro[3.3]heptan-6-yl)benzonitrile (23 mg, 65%) as an off-white solid. LC-MS (ES, m/z)=469.15 [M+1]⁺. ¹H NMR (400 MHz, DMSO-d₆) δ 11.01 (s, 1H), 7.87 (d, J=11.1 Hz, 1H), 7.83-7.70 (m, 4H), 7.43 (dd, J=8.5, 3.1 Hz, 2H), 5.19-5.09 (m, 1H), 4.56-4.33 (m, 3H), 4.24 (d, J=2.7 Hz, 2H), 4.02 (s, 1H), 3.49 (dp, J=26.7, 8.8 Hz, 1H), 2.92 (ddd, J=18.1, 13.5, 5.3 Hz, 1H), 2.62 (t, J=9.1 Hz, 3H), 2.49-2.24 (m, 3H), 2.04 (dd, J=12.4, 6.2 Hz, 1H).

### Example 233

### Synthesis of 3-(5-(6-(4-methoxyphenyl)-2-azaspiro[3.3]heptane-2-carbonyl)-1-oxoisoindolin-2-yl)piperidine-2,6-dione

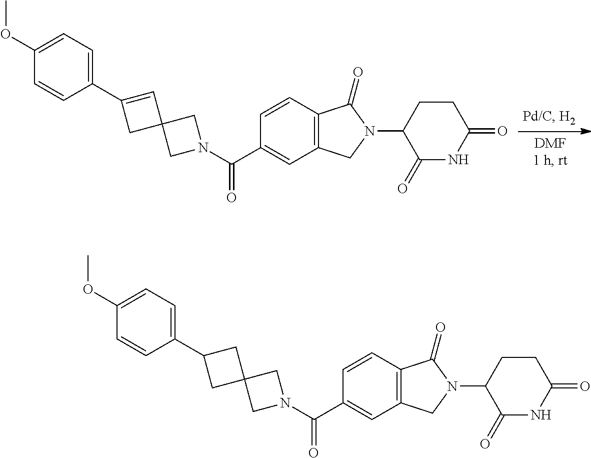 [SMILES: COc1ccc(C2=CC3(C2)CN(C(=O)c2ccc4c(c2)CN(C2CCC(=O)NC2=O)C4=O)C3)cc1 ; COc1ccc(C2CC3(C2)CN(C(=O)c2ccc4c(c2)CN(C2CCC(=O)NC2=O)C4=O)C3)cc1]

Step 1. Preparation of 3-(5-(6-(4-methoxyphenyl)-2-azaspiro[3.3]heptane-2-carbonyl)-1-oxoisoindolin-2-yl)piperidine-2,6-dione. To a stirred mixture of 3-{5-[6-(4-methoxyphenyl)-2-azaspiro[3.3]hept-5-ene-2-carbonyl]-1-oxo-3H-isoindol-2-yl}piperidine-2,6-dione (20 mg, 0.042 mmol, 1 equiv) in DMF (0.5 mL) was added Pd/C (10 mg, Palladium 10% on Carbon (wetted with ca. 55% Water)) at room temperature under hydrogen atmosphere. The resulting mixture was stirred for 1 h at room temperature under hydrogen atmosphere. The resulting mixture was filtered, the filter cake was washed with THF (3×5 mL). The filtrate was concentrated under reduced pressure. The crude product (20 mg) was purified by Prep-HPLC to afford 3-(5-(6-(4-methoxyphenyl)-2-azaspiro[3.3]heptane-2-carbonyl)-1-oxoisoindolin-2-yl)piperidine-2,6-dione (9.1 mg, 45%) as a white solid. LC-MS (ES, m/z)=473.55 [M+1]⁺. ¹H NMR (400 MHz, DMSO-d₆) δ 10.99 (d, J=18.0 Hz, 1H), 7.86-7.43 (m, 3H), 7.34-6.72 (m, 4H), 5.14 (s, 1H), 4.61-4.18 (m, 3H), 4.02-3.37 (m, 6H), 2.91 (s, 3H), 2.41 (s, 2H), 2.03 (s, 2H), 1.24 (s, 1H).

### Example 234

### Synthesis of 3-(5-(6-(3,4-difluorophenyl)-2-azaspiro[3.3]hept-5-ene-2-carbonyl)-1-oxoisoindolin-2-yl)piperidine-2,6-dione

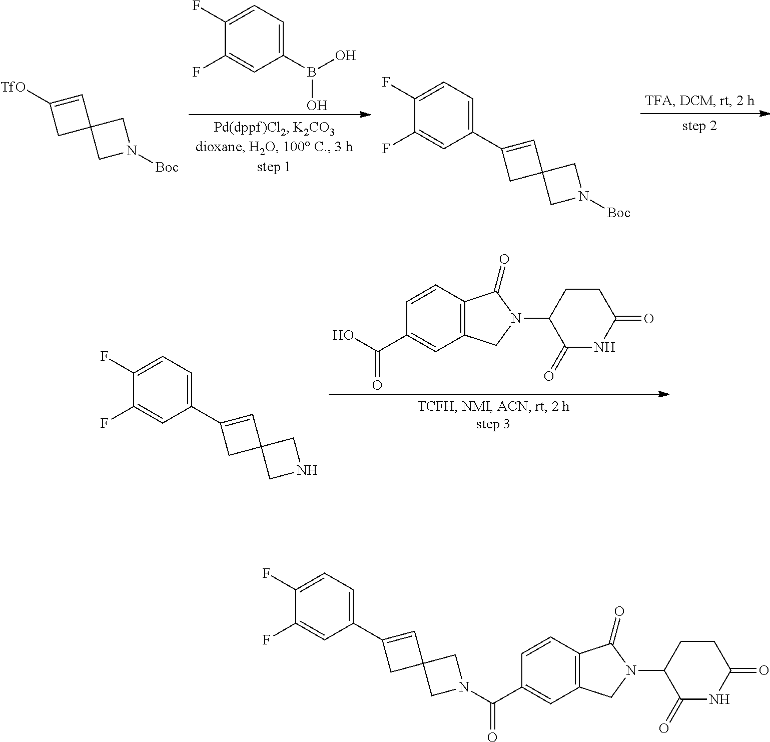 [SMILES: CC(C)(C)OC(=O)N1CC2(C=C(OS(=O)(=O)C(F)(F)F)C2)C1 ; OB(O)c1ccc(F)c(F)c1 ; CC(C)(C)OC(=O)N1CC2(C=C(c3ccc(F)c(F)c3)C2)C1 ; Fc1ccc(C2=CC3(CNC3)C2)cc1F ; O=C1CCC(N2Cc3cc(C(=O)O)ccc3C2=O)C(=O)N1 ; O=C1CCC(N2Cc3cc(C(=O)N4CC5(C=C(c6ccc(F)c(F)c6)C5)C4)ccc3C2=O)C(=O)N1]

Step 1. Preparation of tert-butyl 6-(3,4-difluorophenyl)-2-azaspiro[3.3]hept-5-ene-2-carboxylate. A mixture of tert-butyl 6-(trifluoromethanesulfonyloxy)-2-azaspiro[3.3]hept-5-ene-2-carboxylate (1 g, 2.913 mmol, 1 equiv), 3,4-difluorophenylboronic acid (0.92 g, 5.826 mmol, 2 equiv), Pd(dppf)Cl₂ (0.21 g, 0.291 mmol, 0.1 equiv) and K₂CO₃ (1.21 g, 8.739 mmol, 3 equiv) in dioxane (14 mL) and H₂O (1.4 mL) was stirred for 3 h at 100° C. under nitrogen atmosphere. The mixture was allowed to cool down to room temperature. The resulting mixture was filtered, the filter cake was washed with dioxane (3×10 mL). The filtrate was concentrated under reduced pressure. The residue was purified by reversed-phase flash chromatography to afford 6-(3,4-difluorophenyl)-2-azaspiro[3.3]hept-5-ene-2-carboxylate (350 mg, 39%) as an off-white solid. LC-MS (ES, m/z)=293.05 [M-tBu+ACN+1]⁺.

Step 2. Preparation of 6-(3,4-difluorophenyl)-2-azaspiro[3.3]hept-5-ene. To a stirred mixture of tert-butyl 6-(3,4-difluorophenyl)-2-azaspiro[3.3]hept-5-ene-2-carboxylate (340 mg, 1.106 mmol, 1 equiv) in DCM (2 mL) was added TFA (0.4 mL) dropwise at 0° C. under the air atmosphere. The resulting mixture was stirred for 2 h at room temperature under nitrogen atmosphere. The resulting mixture was concentrated under reduced pressure to afford 6-(3,4-difluorophenyl)-2-azaspiro[3.3]hept-5-ene (450 mg, crude) as a brown oil. The crude product was used in the next step directly without further purification. LC-MS (ES, m/z)=208.05 [M+1]⁺.

Step 3. Preparation of 3-(5-(6-(3,4-difluorophenyl)-2-azaspiro[3.3]hept-5-ene-2-carbonyl)-1-oxoisoindolin-2-yl)piperidine-2,6-dione. To a stirred mixture of 6-(3,4-difluorophenyl)-2-azaspiro[3.3]hept-5-ene (215.66 mg, 1.041 mmol, 1 equiv), 2-(2,6-dioxopiperidin-3-yl)-1-oxo-3H-isoindole-5-carboxylic acid (300 mg, 1.041 mmol, 1.00 equiv) and NMI (512.70 mg, 6.246 mmol, 6 equiv) in DMF (4 mL) was added TCFH (438.01 mg, 1.561 mmol, 1.5 equiv) at room temperature under air atmosphere. The resulting mixture was stirred for 2 h at room temperature under the air atmosphere. The resulting mixture was diluted with water and extracted with EA (3×50 mL). The combined organic layers were washed with brine (3×50 mL), dried over anhydrous Na₂SO₄. After filtration, the filtrate was concentrated under reduced pressure. The residue was purified by reversed-phase flash chromatography to afford 3-(5-(6-(3,4-difluorophenyl)-2-azaspiro[3.3]hept-5-ene-2-carbonyl)-1-oxoisoindolin-2-yl)piperidine-2,6-dione (38 mg, 7.40%) as a pink solid. LC-MS (ES, m/z)=478.15 [M+1]⁺. ¹H NMR (400 MHz, DMSO-d₆) δ 11.01 (s, 1H), 7.89 (s, 1H), 7.79 (t, J=1.5 Hz, 2H), 7.60-7.37 (m, 2H), 7.23 (dt, J=8.4, 2.8 Hz, 1H), 6.60 (s, 1H), 5.14 (dd, J=13.3, 5.1 Hz, 1H), 4.67-4.23 (m, 6H), 3.07-2.82 (m, 3H), 2.73-2.57 (m, 1H), 2.50-2.32 (m, 1H), 2.11-1.98 (m, 1H).

### Example 235

### Synthesis of 3-(5-(6-(2,4-difluorophenyl)-2-azaspiro[3.3]hept-5-ene-2-carbonyl oxoisoindolin-2-yl)piperidine-2,6-dione

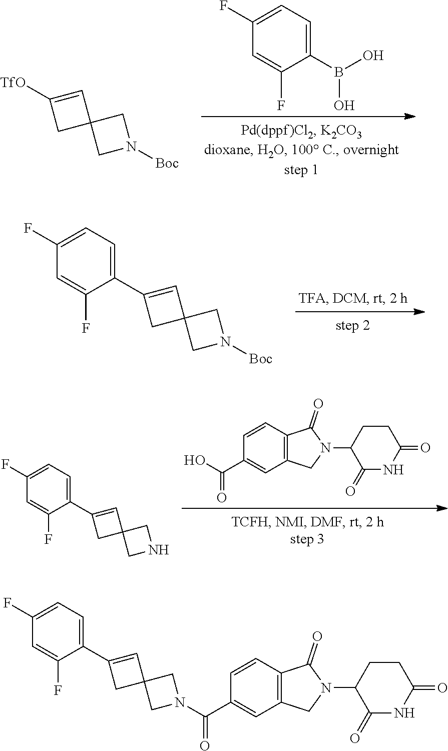 [SMILES: CC(C)(C)OC(=O)N1CC2(C=C(OS(=O)(=O)C(F)(F)F)C2)C1 ; OB(O)c1ccc(F)cc1F ; CC(C)(C)OC(=O)N1CC2(C=C(c3ccc(F)cc3F)C2)C1 ; Fc1ccc(C2=CC3(CNC3)C2)c(F)c1 ; O=C1CCC(N2Cc3cc(C(=O)O)ccc3C2=O)C(=O)N1 ; O=C1CCC(N2Cc3cc(C(=O)N4CC5(C=C(c6ccc(F)cc6F)C5)C4)ccc3C2=O)C(=O)N1]

Step 1. Preparation of tert-butyl 6-(2,4-difluorophenyl)-2-azaspiro[3.3]hept-5-ene-2-carboxylate. A mixture of tert-butyl 6-(trifluoromethanesulfonyloxy)-2-azaspiro[3.3]hept-5-ene-2-carboxylate (550 mg, 1.602 mmol, 1 equiv), 2,4-difluorophenylboronic acid (505.9 mg, 3.204 mmol, 2 equiv), Pd(dppf)Cl₂ (117.2 mg, 0.160 mmol, 0.1 equiv) and K₂CO₃ (664.2 mg, 4.806 mmol, 3 equiv) in dioxane (8 mL) and H₂O (0.8 mL) was stirred for overnight at 100° C. under nitrogen atmosphere. The mixture was allowed to cool down to room temperature. The resulting mixture was diluted with water and extracted with EA (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 6-(2,4-difluorophenyl)-2-azaspiro[3.3]hept-5-ene-2-carboxylate (70 mg, 14%) as a brown oil. LC-MS (ES, m/z)=293.05 [M-tBu+ACN+1]⁺.

Step 2. Preparation of 6-(2,4-difluorophenyl)-2-azaspiro[3.3]hept-5-ene. To a stirred mixture of tert-butyl 6-(2,4-difluorophenyl)-2-azaspiro[3.3]hept-5-ene-2-carboxylate (70 mg, 0.228 mmol, 1 equiv) in DCM (1 mL) was added TFA (0.2 mL) dropwise at 0° C. under air atmosphere. The resulting mixture was stirred for 2 h at room temperature under nitrogen atmosphere. The resulting mixture was concentrated under reduced pressure to afford 6-(2,4-difluorophenyl)-2-azaspiro[3.3]hept-5-ene (110 mg, crude) as a brown oil. The crude product was used in the next step directly without further purification. LC-MS (ES, m/z)=208.05 [M+1]⁺.

Step 3. Preparation of 3-(5-(6-(2,4-difluorophenyl)-2-azaspiro[3.3]hept-5-ene-2-carbonyl)-1-oxoisoindolin-2-yl)piperidine-2,6-dione. To a stirred mixture of 6-(2,4-difluorophenyl)-2-azaspiro[3.3]hept-5-ene (110 mg, 0.531 mmol, 1 equiv), 2-(2,6-dioxopiperidin-3-yl)-1-oxo-3H-isoindole-5-carboxylic acid (153.02 mg, 0.531 mmol, 1 equiv) and NMI (261.50 mg, 3.186 mmol, 6 equiv) in DMF (1.7 mL) was added TCFH (223.41 mg, 0.796 mmol, 1.5 equiv) at room temperature under air atmosphere. The resulting mixture was stirred for 2 h at room temperature under air atmosphere. The resulting mixture was diluted with water and extracted with EA (3×30 mL). The combined organic layers were washed with brine (3×50 mL), dried over anhydrous Na₂SO₄. After filtration, the filtrate was concentrated under reduced pressure. The residue was purified by reversed-phase flash chromatography to afford 3-(5-(6-(2,4-difluorophenyl)-2-azaspiro[3.3]hept-5-ene-2-carbonyl)-1-oxoisoindolin-2-yl)piperidine-2,6-dione (12.6 mg, 4.89%) as a pink solid. LC-MS (ES, m/z)=478.15 [M+1]⁺. ¹H NMR (400 MHz, DMSO-d₆) δ 11.01 (s, 1H), 7.89 (d, J=4.1 Hz, 1H), 7.84-7.72 (m, 2H), 7.39 (td, J=8.7, 6.5 Hz, 1H), 7.30 (ddd, J=11.5, 9.3, 2.6 Hz, 1H), 7.12 (td, J=8.5, 2.6 Hz, 1H), 6.49 (d, J=2.9 Hz, 1H), 5.14 (dd, J=13.2, 5.1 Hz, 1H), 4.63-4.46 (m, 3H), 4.42-4.22 (m, 3H), 3.05 (d, J=3.0 Hz, 2H), 2.97-2.86 (m, 1H), 2.61 (ddd, J=17.2, 4.7, 2.4 Hz, 1H), 2.42 (dd, J=13.1, 4.6 Hz, 1H), 2.10-1.93 (m, 1H).

### Example 236

### Synthesis of 3-(1-oxo-5-(6-(p-tolyl)-2-azaspiro[3.3]hept-5-ene-2-carbonyl)isoindolin-2-yl)piperidine-2,6-dione

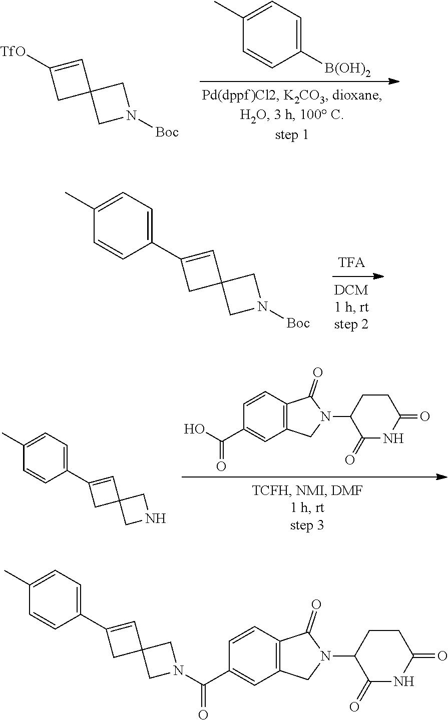

Step 1. Preparation of tert-butyl 6-(p-tolyl)-2-azaspiro[3.3]hept-5-ene-2-carboxylate. To a stirred mixture of tert-butyl 6-(trifluoromethanesulfonyloxy)-2-azaspiro[3.3]hept-5-ene-2-carboxylate (500 mg, 1.456 mmol, 1 equiv) and p-tolueneboronic acid (297.0 mg, 2.184 mmol, 1.5 equiv) in dioxane (10 mL) and H₂O (1 mL) were added Pd(dppf)Cl₂ (106.6 mg, 0.146 mmol, 0.1 equiv) and K₂CO₃ (603.8 mg, 4.369 mmol, 3 equiv) at room temperature under air atmosphere. The resulting mixture was stirred for 3 h at 100° C. under nitrogen atmosphere. The mixture was allowed to cool down to room temperature. The resulting mixture was diluted with water (50 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to afford tert-butyl 6-(p-tolyl)-2-azaspiro[3.3]hept-5-ene-2-carboxylate (76 mg, 18%) as a yellow solid. LC-MS (ES, m/z)=271.10 [M-tBu+ACN+1]⁺.

Step 2. Preparation of 6-(p-tolyl)-2-azaspiro[3.3]hept-5-ene. To a stirred solution of tert-butyl 6-(p-tolyl)-2-azaspiro[3.3]hept-5-ene-2-carboxylate (76 mg, 0.267 mmol, 1 equiv) in DCM (2 mL) was added TFA (0.4 mL) dropwise at 0° C. under nitrogen atmosphere. The resulting mixture was stirred for 1 h at room temperature under nitrogen atmosphere. The resulting mixture was concentrated under reduced pressure. This resulted in 6-(p-tolyl)-2-azaspiro[3.3]hept-5-ene (100 mg, crude) as a brown oil. The crude product was used in the next step directly without further purification. LC-MS (ES, m/z)=186.10 [M+1]⁺.

Step 3. Preparation of 3-(1-oxo-5-(6-(p-tolyl)-2-azaspiro[3.3]hept-5-ene-2-carbonyl)isoindolin-2-yl)piperidine-2,6-dione. To a stirred mixture of 2-(2,6-dioxopiperidin-3-yl)-1-oxo-3H-isoindole-5-carboxylic acid (100 mg, 0.347 mmol, 1 equiv) and 6-(4-methylphenyl)-2-azaspiro[3.3]hept-5-ene (64.3 mg, 0.347 mmol, 1 equiv) in DMF (1 mL) were added NMI (142.4 mg, 1.734 mmol, 5 equiv) and TCFH (146.0 mg, 0.520 mmol, 1.5 equiv) in portions at room temperature under air atmosphere. The resulting mixture was stirred for 1 h at room temperature under nitrogen atmosphere. The resulting mixture was diluted with water (50 mL) and extracted with EtOAc (3×20 mL). The combined organic layers were washed with brine (2×30 mL), dried over anhydrous Na₂SO₄. After filtration, the filtrate was concentrated under reduced pressure. The residue was purified by reversed-phase flash chromatography to afford 3-(1-oxo-5-(6-(p-tolyl)-2-azaspiro[3.3]hept-5-ene-2-carbonyl)isoindolin-2-yl)piperidine-2,6-dione (11 mg, 6.71%) as an off-white solid. LC-MS (ES, m/z)=456.20 [M+1]⁺. ¹H NMR (400 MHz, DMSO-d₆) δ 11.01 (s, 1H), 7.91-7.71 (m, 3H), 7.31-7.15 (m, 3H), 7.09 (d, J=2.9 Hz, 1H), 6.46 (s, 1H), 5.14 (dd, J=13.3, 5.1 Hz, 1H), 4.57-4.22 (m, 6H), 3.30-3.22 (m, 1H), 2.97 (d, J=2.5 Hz, 1H), 2.94-2.86 (m, 1H), 2.61 (ddd, J=17.3, 4.4, 2.2 Hz, 1H), 2.42 (qd, J=13.1, 4.4 Hz, 1H), 2.30 (s, 2H), 2.25 (d, J=2.5 Hz, 1H), 2.07-1.97 (m, 1H).

### Example 237

### Synthesis of 3-(5-(6-(4-methoxyphenyl)-2-azaspiro[3.3]hept-5-ene-2-carbonyl)-1-oxoisoindolin-2-yl)piperidine-2,6-dione

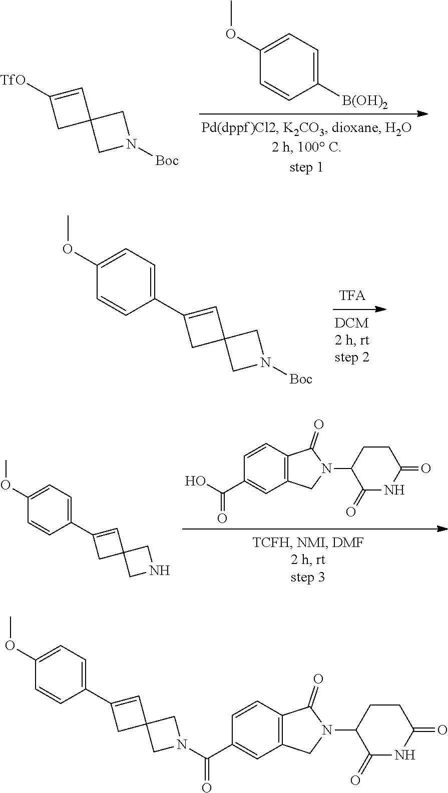 [SMILES: CC(C)(C)OC(=O)N1CC2(C=C(OS(=O)(=O)C(F)(F)F)C2)C1 ; COc1ccc(B(O)O)cc1 ; COc1ccc(C2=CC3(C2)CN(C(=O)OC(C)(C)C)C3)cc1 ; COc1ccc(C2=CC3(CNC3)C2)cc1 ; O=C1CCC(N2Cc3cc(C(=O)O)ccc3C2=O)C(=O)N1 ; COc1ccc(C2=CC3(C2)CN(C(=O)c2ccc4c(c2)CN(C2CCC(=O)NC2=O)C4=O)C3)cc1]

Step 1. Preparation of tert-butyl 6-(4-methoxyphenyl)-2-azaspiro[3.3]hept-5-ene-2-carboxylate. To a stirred mixture of tert-butyl 6-(trifluoromethanesulfonyloxy)-2-azaspiro[3.3]hept-5-ene-2-carboxylate (500 mg, 1.456 mmol, 1 equiv) and 4-methoxyphenylboronic acid (331.9 mg, 2.184 mmol, 1.5 equiv) in dioxane (10 mL) and H₂O (1 mL) were added Pd(dppf)Cl₂ (106.5 mg, 0.146 mmol, 0.1 equiv) and K₂CO₃ (603.8 mg, 4.368 mmol, 3 equiv) at room temperature under nitrogen atmosphere. The resulting mixture was stirred for 2 h at 100° C. under nitrogen atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6-(4-methoxyphenyl)-2-azaspiro[3.3]hept-5-ene-2-carboxylate (120 mg, 27%) as a yellow oil. LC-MS (ES, m/z)=287.10 [M-tBu+ACN+1]⁺.

Step 2. Preparation of 6-(4-methoxyphenyl)-2-azaspiro[3.3]hept-5-ene. To a stirred mixture of tert-butyl 6-(4-methoxyphenyl)-2-azaspiro[3.3]hept-5-ene-2-carboxylate (120 mg, 0.398 mmol, 1 equiv) in DCM (1 ml) was added TFA (0.2 mL, 2.693 mmol, 6.76 equiv) dropwise at 0° C. The resulting mixture was stirred for 2 h at room temperature. The resulting mixture was concentrated under reduced pressure. The residue was purified by reversed-phase flash chromatography to afford 6-(4-methoxyphenyl)-2-azaspiro[3.3]hept-5-ene (80 mg, 100%) as a yellow solid. LC-MS (ES, m/z)=222.70 [M+Na]⁺.

Step 3. Preparation of 3-(5-(6-(4-methoxyphenyl)-2-azaspiro[3.3]hept-5-ene-2-carbonyl)-1-oxoisoindolin-2-yl)piperidine-2,6-dione. To a stirred mixture of 6-(4-methoxyphenyl)-2-azaspiro[3.3]hept-5-ene (70 mg, 0.348 mmol, 1 equiv) and 2-(2,6-dioxopiperidin-3-yl)-1-oxo-3H-isoindole-5-carboxylic acid (100.2 mg, 0.348 mmol, 1 equiv) in DMF (1 mL) were added TCFH (146.3 mg, 0.522 mmol, 1.5 equiv) and NMI (114.2 mg, 1.392 mmol, 4 equiv) at room temperature. The resulting mixture was stirred for 2 h at room temperature. The resulting mixture was diluted with water (50 mL) and extracted with EtOAc (3×20 mL). The combined organic layers were washed with brine (2×50 mL) and concentrated under reduced pressure. The crude product (50 mg) was purified by Prep-HPLC to afford 3-(5-(6-(4-methoxyphenyl)-2-azaspiro[3.3]hept-5-ene-2-carbonyl)-1-oxoisoindolin-2-yl)piperidine-2,6-dione (9 mg, 5.61%) as a white solid. LC-MS (ES, m/z)=472.45 [M+1]⁺. ¹H NMR (400 MHz, DMSO-d₆) δ 10.99 (d, J=5.3 Hz, 1H), 7.82-7.62 (m, 3H), 7.37 (dd, J=28.5, 8.4 Hz, 1H), 7.20 (d, J=8.8 Hz, 1H), 6.96-6.75 (m, 2H), 5.12 (dd, J=13.1, 6.4 Hz, 1H), 4.55-4.31 (m, 3H), 4.25-4.06 (m, 2H), 3.94 (dd, J=23.2, 9.5 Hz, 2H), 3.77-3.61 (m, 3H), 2.98-2.69 (m, 4H), 2.61 (d, J=16.8 Hz, 1H), 2.44-2.31 (m, 1H), 2.02 (s, 1H).

### Example 238

### Synthesis of 2-(2-(2-(2,6-dioxopiperidin-3-yl)-1-oxoisoindoline-5-carbonyl)-2-azaspiro[3.3]hept-5-en-6-yl)benzonitrile

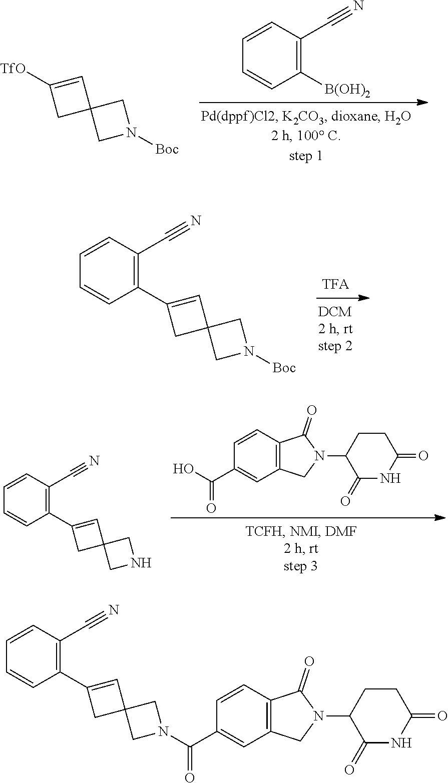

Step 1. Preparation of tert-butyl 6-(2-cyanophenyl)-2-azaspiro[3.3]hept-5-ene-2-carboxylate. To a stirred mixture of tert-butyl 6-(trifluoromethanesulfonyloxy)-2-azaspiro[3.3]hept-5-ene-2-carboxylate (500 mg, 1.456 mmol, 1 equiv) and 2-cyanophenylboronic acid (321.0 mg, 2.184 mmol, 1.5 equiv) in dioxane (10 mL) and H₂O (1.00 mL) were added Pd(dppf)Cl₂ (106.5 mg, 0.146 mmol, 0.1 equiv) and K₂CO₃ (603.8 mg, 4.368 mmol, 3 equiv) at room temperature under nitrogen atmosphere. The resulting mixture was stirred for 2 h at 100° C. under nitrogen atmosphere. The resulting mixture was diluted with water (50 mL) and extracted with EtOAc (3×30 mL). The combined organic layers were washed with brine (2×30 mL), dried over anhydrous Na₂SO₄. After filtration, the filtrate was concentrated under reduced pressure. The residue was purified by reversed-phase flash chromatography to afford tert-butyl 6-(2-cyanophenyl)-2-azaspiro[3.3]hept-5-ene-2-carboxylate (200 mg, 46%) as a yellow oil. LC-MS (ES, m/z)=282.05 [M-tBu+ACN+1]⁺.

Step 2. Preparation of 2-(2-azaspiro[3.3]hept-5-en-6-yl)benzonitrile. To a stirred mixture of tert-butyl 6-(2-cyanophenyl)-2-azaspiro[3.3]hept-5-ene-2-carboxylate (200 mg, 0.675 mmol, 1 equiv) in DCM (2 mL) was added TFA (0.4 mL, 5.385 mmol, 7.98 equiv) dropwise at 0° C. The resulting mixture was stirred for 2 h at room temperature. The resulting mixture was concentrated under reduced pressure. The residue was purified by reversed-phase flash chromatography to afford 2-(2-azaspiro[3.3]hept-5-en-6-yl)benzonitrile (100 mg, 76%) as a yellow solid. LC-MS (ES, m/z)=197.05 [M+1]⁺.

Step 3. Preparation of 2-(2-(2-(2,6-dioxopiperidin-3-yl)-1-oxoisoindoline-5-carbonyl)-2-azaspiro[3.3]hept-5-en-6-yl)benzonitrile. To a stirred mixture of 2-(2-azaspiro[3.3]hept-5-en-6-yl)benzonitrile (90 mg, 0.459 mmol, 1 equiv) and 2-(2,6-dioxopiperidin-3-yl)-1-oxo-3H-isoindole-5-carboxylic acid (132.1 mg, 0.459 mmol, 1 equiv) in DMF (2 mL) were added TCFH (193.0 mg, 0.689 mmol, 1.5 equiv) and NMI (150.6 mg, 1.836 mmol, 4 equiv) at room temperature. The resulting mixture was stirred for 2 h at room temperature. The resulting mixture was diluted with water (30 mL) and extracted with EtOAc (3×20 mL). The combined organic layers were washed with brine (2×30 mL) and concentrated under reduced pressure. The crude product (50 mg) was purified by Prep-HPLC to afford 2-(2-(2-(2,6-dioxopiperidin-3-yl)-1-oxoisoindoline-5-carbonyl)-2-azaspiro[3.3]hept-5-en-6-yl)benzonitrile (4.3 mg, 2%) as a white solid. LC-MS (ES, m/z)=467.15 [M+1]⁺. ¹H NMR (400 MHz, DMSO-d₆) δ 11.01 (s, 1H), 7.92-7.70 (m, 5H), 7.58-7.46 (m, 2H), 6.94 (s, 1H), 5.14 (dd, J=13.3, 5.1 Hz, 1H), 4.61-4.48 (m, 3H), 4.43-4.28 (m, 3H), 3.17 (d, J=3.3 Hz, 2H), 2.92 (ddd, J=18.2, 13.5, 5.5 Hz, 1H), 2.61 (d, J=16.9 Hz, 1H), 2.46-2.35 (m, 1H), 2.07-1.99 (m, 1H).

### Example 239

### Synthesis of 2-(2-(2-(2,6-dioxopiperidin-3-yl)-1-oxoisoindoline-5-carbonyl azaspiro[3.3]heptan-6-yl)benzonitrile

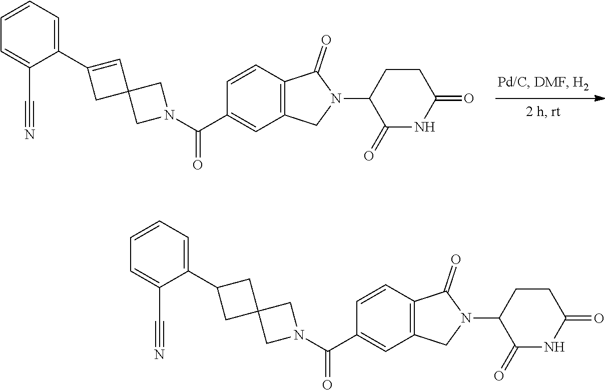 [SMILES: N#Cc1ccccc1C1=CC2(C1)CN(C(=O)c1ccc3c(c1)CN(C1CCC(=O)NC1=O)C3=O)C2 ; N#Cc1ccccc1C1CC2(C1)CN(C(=O)c1ccc3c(c1)CN(C1CCC(=O)NC1=O)C3=O)C2]

Step 1. Preparation of 2-(2-(2-(2,6-dioxopiperidin-3-yl)-1-oxoisoindoline-5-carbonyl)-2-azaspiro[3.3]heptan-6-yl)benzonitrile. To a stirred mixture of 2-{2-[2-(2,6-dioxopiperidin-3-yl)-1-oxo-3H-isoindole-5-carbonyl]-2-azaspiro[3.3]hept-5-en-6-yl}benzonitrile (20 mg, 0.043 mmol, 1 equiv) in DMF (1 mL) was added Pd/C (10 mg, Palladium 10% on Carbon (wetted with ca. 55% Water)) at room temperature under hydrogen atmosphere. The resulting mixture was stirred for 2 h at room temperature under hydrogen atmosphere. The resulting mixture was filtered, the filter cake was washed with THF (2×5 mL). The filtrate was concentrated under reduced pressure. The crude product (20 mg) was purified by Prep-HPLC to afford 2-(2-(2-(2,6-dioxopiperidin-3-yl)-1-oxoisoindoline-5-carbonyl)-2-azaspiro[3.3]heptan-6-yl)benzonitrile (3 mg, 14%) as a white solid. LC-MS (ES, m/z)=469.15 [M+1]. ¹H NMR (400 MHz, DMSO-d₆) δ 11.01 (s, 1H), 7.87 (d, J=14.7 Hz, 1H), 7.81-7.64 (m, 4H), 7.55 (dd, J=18.0, 8.1 Hz, 1H), 7.41 (d, J=7.8 Hz, 1H), 5.14 (d, J=12.7 Hz, 1H), 4.55-4.46 (m, 2H), 4.39 (dd, J=17.2, 7.9 Hz, 1H), 4.27 (s, 2H), 4.03 (s, 1H), 3.69 (dd, J=19.9, 8.9 Hz, 1H), 2.90 (d, J=14.4 Hz, 1H), 2.72 (d, J=9.6 Hz, 2H), 2.61 (d, J=17.0 Hz, 1H), 2.41 (d, J=10.7 Hz, 2H), 2.03 (s, 1H), 1.24 (s, 1H).

### Example 240

### Synthesis of 3-(5-(6-(3-fluoro-4-(trifluoromethyl)phenyl)-2-azaspiro[3.3]hept-5-ene-2-carbonyl)-1-oxoisoindolin-2-yl)piperidine-2,6-dione

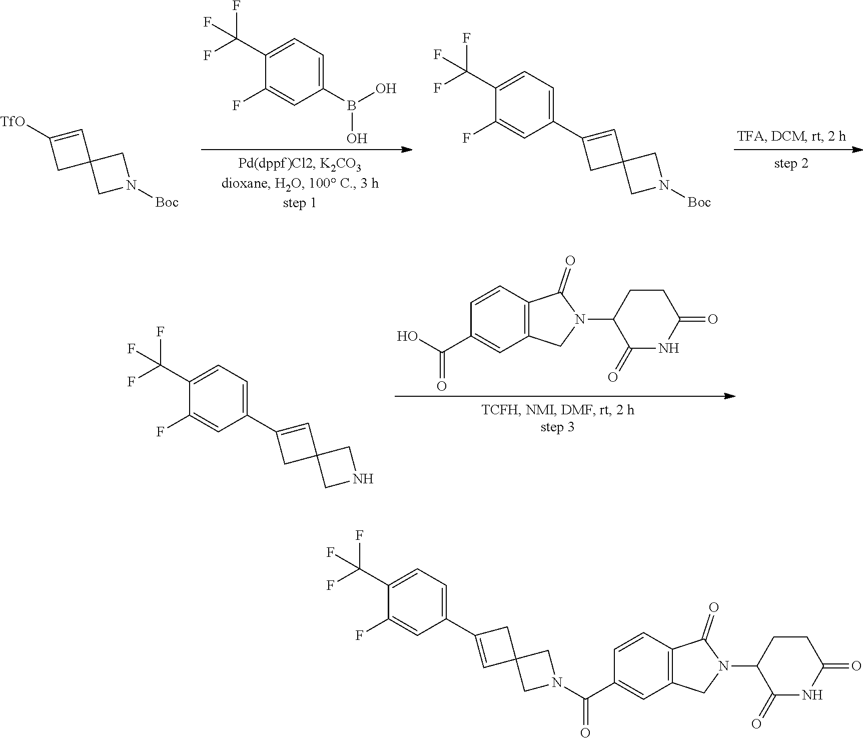 [SMILES: CC(C)(C)OC(=O)N1CC2(C=C(OS(=O)(=O)C(F)(F)F)C2)C1 ; OB(O)c1ccc(C(F)(F)F)c(F)c1 ; CC(C)(C)OC(=O)N1CC2(C=C(c3ccc(C(F)(F)F)c(F)c3)C2)C1 ; Fc1cc(C2=CC3(CNC3)C2)ccc1C(F)(F)F ; O=C1CCC(N2Cc3cc(C(=O)O)ccc3C2=O)C(=O)N1 ; O=C1CCC(N2Cc3cc(C(=O)N4CC5(C=C(c6ccc(C(F)(F)F)c(F)c6)C5)C4)ccc3C2=O)C(=O)N1]

Step 1. Preparation of tert-butyl 6-(3-fluoro-4-(trifluoromethyl)phenyl)-2-azaspiro[3.3]hept-5-ene-2-carboxylate. A mixture of tert-butyl 6-(trifluoromethanesulfonyloxy)-2-azaspiro[3.3]hept-5-ene-2-carboxylate (600 mg, 1.748 mmol, 1 equiv), 3-fluoro-4-(trifluoromethyl)phenylboronic acid (726.7 mg, 3.496 mmol, 2 equiv), Pd(dppf)Cl₂ (127.9 mg, 0.175 mmol, 0.1 equiv) and K₂CO₃ (724.6 mg, 5.244 mmol, 3 equiv) in dioxane (9 mL) and H₂O (0.9 mL) was stirred for 3 h at 100° C. under nitrogen atmosphere. The mixture was allowed to cool down to room temperature. The resulting mixture was filtered, the filter cake was washed with dioxane (3×10 mL). The filtrate was concentrated under reduced pressure. The residue was purified by reversed-phase flash chromatography to afford tert-butyl 6-[3-fluoro-4-(trifluoromethyl)phenyl]-2-azaspiro[3.3]hept-5-ene-2-carboxylate (280 mg, 45%) as a brown oil. LC-MS (ES, m/z)=343.05 [M-tBu+ACN+1]+.

Step 2. Preparation of 6-(3-fluoro-4-(trifluoromethyl)phenyl)-2-azaspiro[3.3]hept-5-ene. To a stirred mixture of tert-butyl 6-[3-fluoro-4-(trifluoromethyl)phenyl]-2-azaspiro[3.3]hept-5-ene-2-carboxylate (280 mg, 0.784 mmol, 1 equiv) in DCM (5 mL) was added TFA (1 mL) dropwise at 0° C. under nitrogen atmosphere. The resulting mixture was stirred for 2 h at room temperature under nitrogen atmosphere. The resulting mixture was concentrated under reduced pressure to afford 6-(3-fluoro-4-(trifluoromethyl)phenyl)-2-azaspiro[3.3]hept-5-ene (300 mg, crude) as a brown oil. The crude product was used in the next step directly without further purification. LC-MS (ES, m/z)=299.05 [M+ACN+1]⁺.

Step 3. Preparation of 3-(5-(6-(3-fluoro-4-(trifluoromethyl)phenyl)-2-azaspiro[3.3]hept-5-ene-2-carbonyl)-1-oxoisoindolin-2-yl)piperidine-2,6-dione. To a stirred mixture of 6-[3-fluoro-4-(trifluoromethyl)phenyl]-2-azaspiro[3.3]hept-5-ene (100 mg, 0.389 mmol, 1 equiv), 2-(2,6-dioxopiperidin-3-yl)-1-oxo-3H-isoindole-5-carboxylic acid (112.1 mg, 0.389 mmol, 1 equiv) and NMI (47.9 mg, 0.584 mmol, 1.5 equiv) in DMF (4.5 mL) was added TCFH (545.4 mg, 1.945 mmol, 5 equiv) in portions at room temperature under air atmosphere. The resulting mixture was stirred for 2 h at room temperature under the air atmosphere. The resulting mixture was diluted with water (30 mL) and extracted with EtOAc (3×10 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(5-(6-(3-fluoro-4-(trifluoromethyl)phenyl)-2-azaspiro[3.3]hept-5-ene-2-carbonyl)-1-oxoisoindolin-2-yl)piperidine-2,6-dione (37 mg, 18%) as an off-white solid. LC-MS (ES, m/z)=528.20 [M+1]⁺. ¹H NMR (400 MHz, DMSO-d₆) δ 11.01 (s, 1H), 7.90 (d, J=1.2 Hz, 1H), 7.82-7.73 (m, 3H), 7.57 (d, J=11.7 Hz, 1H), 7.40 (d, J=8.1 Hz, 1H), 6.85 (s, 1H), 5.14 (dd, J=13.3, 5.1 Hz, 1H), 4.61-4.44 (m, 3H), 4.34-4.25 (m, 2H), 3.03 (d, J=2.9 Hz, 2H), 2.94-2.82 (m, 1H), 2.66-2.53 (m, 1H), 2.42 (qd, J=13.2, 4.5 Hz, 1H), 2.04-1.94 (m, 1H).

### Example 241

### Synthesis of 3-(5-(6-(2-fluoro-4-(trifluoromethyl)phenyl)-2-azaspiro[3.3]hept-5-ene-2-carbonyl)-1-oxoisoindolin-2-yl)piperidine-2,6-dione

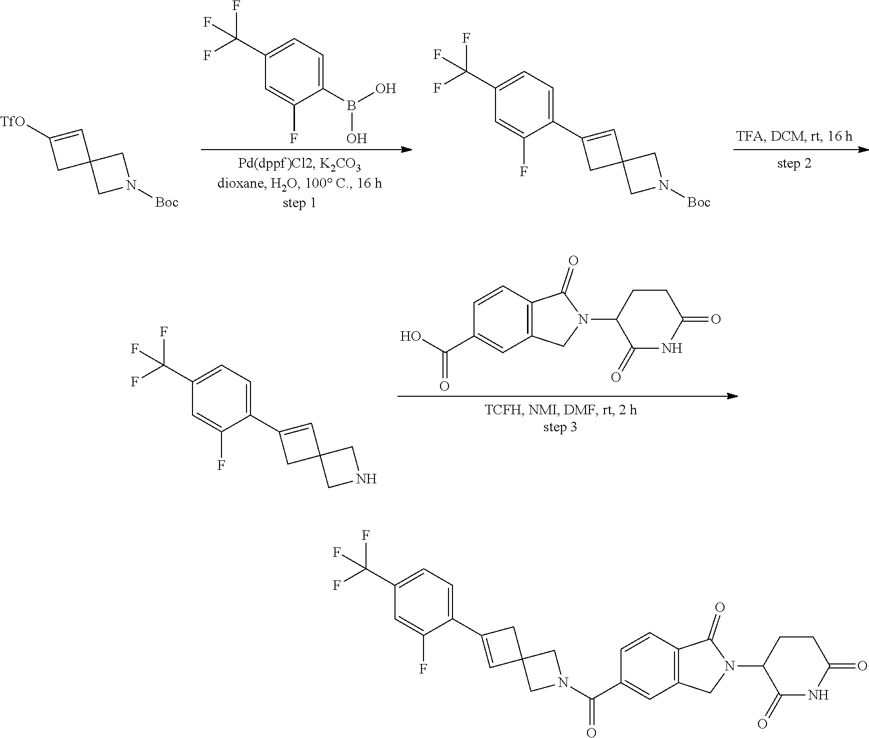 [SMILES: CC(C)(C)OC(=O)N1CC2(C=C(OS(=O)(=O)C(F)(F)F)C2)C1 ; OB(O)c1ccc(C(F)(F)F)cc1F ; CC(C)(C)OC(=O)N1CC2(C=C(c3ccc(C(F)(F)F)cc3F)C2)C1 ; Fc1cc(C(F)(F)F)ccc1C1=CC2(CNC2)C1 ; O=C1CCC(N2Cc3cc(C(=O)O)ccc3C2=O)C(=O)N1 ; O=C1CCC(N2Cc3cc(C(=O)N4CC5(C=C(c6ccc(C(F)(F)F)cc6F)C5)C4)ccc3C2=O)C(=O)N1]

Step 1. Preparation of tert-butyl 6-(2-fluoro-4-(trifluoromethyl)phenyl)-2-azaspiro[3.3]hept-5-ene-2-carboxylate. To a stirred solution of tert-butyl 6-(trifluoromethanesulfonyloxy)-2-azaspiro[3.3]hept-5-ene-2-carboxylate (550 mg, 1.602 mmol, 1 equiv) and 2-fluoro-4-(trifluoromethyl)phenylboronic acid (566.25 mg, 2.723 mmol, 1.7 equiv) in dioxane (5 mL) and H₂O (1 mL) were added K₂CO₃ (664.21 mg, 4.806 mmol, 3 equiv) and Pd(dppf)Cl₂ (117.22 mg, 0.160 mmol, 0.1 equiv) at room temperature. The resulting mixture was stirred overnight at 100° C. under nitrogen atmosphere. The resulting mixture was diluted with water (100 mL). The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6-[2-fluoro-4-(trifluoromethyl)phenyl]-2-azaspiro[3.3]hept-5-ene-2-carboxylate (86 mg, 15%) as a brown solid. LC-MS (ES, m/z)=343.0 [M-tBu+ACN+1]+.

Step 2. Preparation of 6-(2-fluoro-4-(trifluoromethyl)phenyl)-2-azaspiro[3.3]hept-5-ene. To a stirred solution of tert-butyl 6-[2-fluoro-4-(trifluoromethyl)phenyl]-2-azaspiro[3.3]hept-5-ene-2-carboxylate (68 mg, 0.190 mmol, 1 equiv) in DCM (1 mL) was added TFA (0.2 mL) dropwise at 0° C. The resulting mixture was stirred overnight at room temperature. The resulting mixture was concentrated under reduced pressure to afford 6-[2-fluoro-4-(trifluoromethyl)phenyl]-2-azaspiro[3.3]hept-5-ene (56 mg, crude) as a brown oil. The crude product was used in the next step directly without further purification. LC-MS (ES, m/z)=258.10 [M+1]⁺.

Step 3. Preparation of 3-(5-(6-(2-fluoro-4-(trifluoromethyl)phenyl)-2-azaspiro[3.3]hept-5-ene-2-carbonyl)-1-oxoisoindolin-2-yl)piperidine-2,6-dione. To a stirred solution of 2-(2,6-dioxopiperidin-3-yl)-1-oxo-3H-isoindole-5-carboxylic acid (46 mg, 0.160 mmol, 1 equiv) and 6-[2-fluoro-4-(trifluoromethyl)phenyl]-2-azaspiro[3.3]hept-5-ene (49.26 mg, 0.192 mmol, 1.2 equiv) in DMF (0.5 mL) were added NMI (39.31 mg, 0.480 mmol, 3 equiv) and TCFH (67.16 mg, 0.240 mmol, 1.5 equiv) in portions at room temperature under nitrogen atmosphere. The resulting mixture was stirred for 2 h at room temperature. The resulting mixture was diluted with water (20 mL) and extracted with EtOAc (3×10 mL). The combined organic layers were washed with brine (2×20 mL) and concentrated under reduced pressure. The residue was purified by reversed-phase flash chromatography to afford 3-(5-(6-(2-fluoro-4-(trifluoromethyl)phenyl)-2-azaspiro[3.3]hept-5-ene-2-carbonyl)-1-oxoisoindolin-2-yl)piperidine-2,6-dione (67 mg, 80%) as an off-white solid. LC-MS (ES, m/z)=528.15 [M+1]⁺. ¹H NMR (400 MHz, DMSO-d₆) δ 11.00 (s, 1H), 7.89 (d, J=1.3 Hz, 1H), 7.84-7.68 (m, 3H), 7.64-7.50 (m, 2H), 6.73 (d, J=2.9 Hz, 1H), 5.14 (dd, J=13.3, 5.1 Hz, 1H), 4.62-4.47 (m, 3H), 4.42-4.27 (m, 3H), 3.11 (d, J=3.7 Hz, 2H), 2.91 (ddd, J=13.6, 11.1, 6.8 Hz, 1H), 2.61 (ddd, J=17.3, 4.5, 2.4 Hz, 1H), 2.40 (td, J=13.2, 4.5 Hz, 1H), 2.03 (dtd, J=12.5, 5.1, 2.1 Hz, 1H).

### Example 242

### Synthesis of 4-(2-(2-(2,6-dioxopiperidin-3-yl)-1-oxoisoindoline-5-carbonyl)-2-azaspiro[3.3]hept-5-en-6-yl)benzonitrile

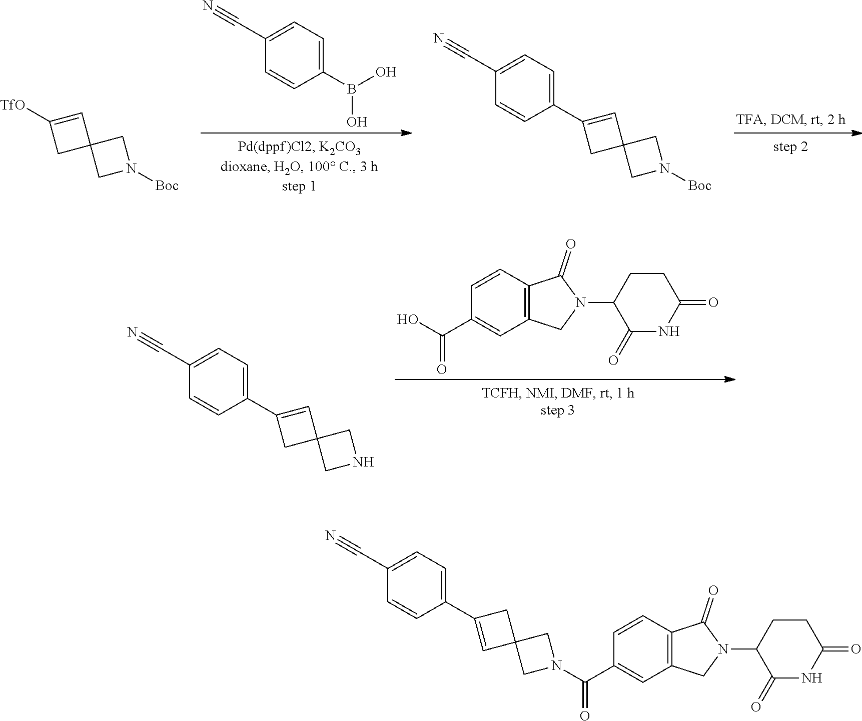 [SMILES: CC(C)(C)OC(=O)N1CC2(C=C(OS(=O)(=O)C(F)(F)F)C2)C1 ; N#Cc1ccc(B(O)O)cc1 ; CC(C)(C)OC(=O)N1CC2(C=C(c3ccc(C#N)cc3)C2)C1 ; N#Cc1ccc(C2=CC3(CNC3)C2)cc1 ; O=C1CCC(N2Cc3cc(C(=O)O)ccc3C2=O)C(=O)N1 ; N#Cc1ccc(C2=CC3(C2)CN(C(=O)c2ccc4c(c2)CN(C2CCC(=O)NC2=O)C4=O)C3)cc1]

Step 1. Preparation of tert-butyl 6-(4-cyanophenyl)-2-azaspiro[3.3]hept-5-ene-2-carboxylate. To a stirred mixture of tert-butyl 6-(trifluoromethanesulfonyloxy)-2-azaspiro[3.3]hept-5-ene-2-carboxylate (500 mg, 1.456 mmol, 1 equiv) and 4-cyanophenylboronic acid (321 mg, 2.185 mmol, 1.5 equiv) in dioxane (10 mL) and H₂O (1 mL) were added Pd(dppf)Cl₂ (106.6 mg, 0.146 mmol, 0.1 equiv) and K₂CO₃ (603.8 mg, 4.369 mmol, 3 equiv) at room temperature under air atmosphere. The resulting mixture was stirred for 3 h at 100° C. under nitrogen atmosphere. The mixture was allowed to cool down to room temperature. The resulting mixture was diluted with water (50 mL) and extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography to afford tert-butyl 6-(4-cyanophenyl)-2-azaspiro[3.3]hept-5-ene-2-carboxylate (103 mg, 24%) as a yellow solid. LC-MS (ES, m/z)=412.30 [M-tBu+ACN+1]⁺.

Step 2. Preparation of 4-(2-azaspiro[3.3]hept-5-en-6-yl)benzonitrile. To a stirred solution of tert-butyl 6-(4-cyanophenyl)-2-azaspiro[3.3]hept-5-ene-2-carboxylate (103 mg, 0.348 mmol, 1 equiv) in DCM (2 mL) was added TFA (0.4 mL, 5.385 mmol, 15.50 equiv) dropwise at 0° C. under nitrogen atmosphere. The resulting mixture was stirred for 2 h at room temperature under nitrogen atmosphere. The resulting mixture was concentrated under vacuum to afford 4-(2-azaspiro[3.3]hept-5-en-6-yl)benzonitrile (90 mg, crude) as a brown oil. The crude product was used in the next step directly without further purification. LC-MS (ES, m/z)=197.05 [M+1]⁺.

Step 3. Preparation of 4-(2-(2-(2,6-dioxopiperidin-3-yl)-1-oxoisoindoline-5-carbonyl)-2-azaspiro[3.3]hept-5-en-6-yl)benzonitrile. To a stirred mixture of 2-(2,6-dioxopiperidin-3-yl)-1-oxo-3H-isoindole-5-carboxylic acid (100 mg, 0.347 mmol, 1 equiv) and 4-(2-azaspiro[3.3]hept-5-en-6-yl)benzonitrile (102.1 mg, 0.520 mmol, 1.5 equiv) in DMF (2 mL) were added NMI (142.4 mg, 1.734 mmol, 5 equiv) and TCFH (146 mg, 0.520 mmol, 1.5 equiv) in portions at room temperature under air atmosphere. The resulting mixture was stirred for 1 h at room temperature under nitrogen atmosphere. The resulting mixture was diluted with water (20 mL) and extracted with EtOAc (3×10 mL). The combined organic layers were washed with brine (2×20 mL) and concentrated under reduced pressure. The residue was purified by reversed-phase flash chromatography to give the crude product (35 mg), which was purified by Prep-HPLC to afford 4-(2-(2-(2,6-dioxopiperidin-3-yl)-1-oxoisoindoline-5-carbonyl)-2-azaspiro[3.3]hept-5-en-6-yl)benzonitrile (3.1 mg, 1.92%) as an off-white solid. LC-MS (ES, m/z)=467.20 [M+1]⁺. ¹H NMR (400 MHz, DMSO-d₆) δ 10.93 (s, 1H), 7.86-7.67 (m, 5H), 7.51 (d, J=8.0 Hz, 2H), 6.73 (s, 1H), 5.07 (dd, J=13.3, 5.1 Hz, 1H), 4.45 (dq, J=15.0, 8.6, 8.1 Hz, 3H), 4.36-4.15 (m, 3H), 2.96 (d, J=3.0 Hz, 2H), 2.85 (ddd, J=18.2, 13.6, 5.3 Hz, 1H), 2.54 (d, J=17.3 Hz, 1H), 2.35 (qd, J=13.2, 4.4 Hz, 1H), 1.97 (dd, J=12.0, 6.0 Hz, 1H).

### Example 243

### Synthesis of 3-(5-(6-(4-fluorophenyl)-6-hydroxy-2-azaspiro[3.3]heptane-2-carbonyl oxoisoindolin-2-yl)piperidine-2,6-dione

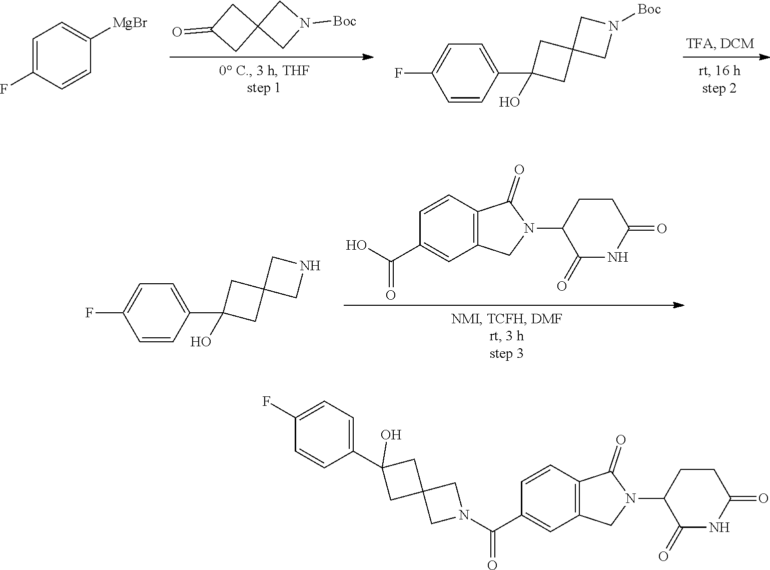

Step 1. Preparation of tert-butyl 6-(4-fluorophenyl)-6-hydroxy-2-azaspiro[3.3]heptane-2-carboxylate. To a stirred solution of tert-butyl 6-oxo-2-azaspiro[3.3]heptane-2-carboxylate (1.0 g, 4.733 mmol, 1.00 equiv) in tetrahydrofuran (20 mL) were added bromo(4-fluorophenyl)magnesium (5.68 mL, 5.680 mmol, 1.2 equiv) dropwise at 0° C. under nitrogen atmosphere. The resulting mixture was stirred for 3 h at 0° C. under nitrogen atmosphere. The reaction was quenched with Water/Ice at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 6-(4-fluorophenyl)-6-hydroxy-2-azaspiro[3.3]heptane-2-carboxylate (560 mg, 38%) as a white solid. LC-MS (ES, m/z)=293.05 [M-tBu+ACN+1]⁺.

Step 2. Preparation of 6-(4-fluorophenyl)-2-azaspiro[3.3]heptan-6-ol. To a stirred solution of tert-butyl 6-(4-fluorophenyl)-6-hydroxy-2-azaspiro[3.3]heptane-2-carboxylate (540 mg, 1.757 mmol, 1 equiv) in DCM (5 mL) was added TFA (1 mL) dropwise at 0° C. The resulting mixture was stirred overnight at room temperature. The resulting mixture was concentrated under reduced pressure to afford 6-(4-fluorophenyl)-2-azaspiro[3.3]heptan-6-ol (420 mg, crude) as a brown oil. The crude product was used in the next step directly without further purification. LC-MS (ES, m/z)=208.15 [M+1]⁺.

Step 3. Preparation of 3-(5-(6-(4-fluorophenyl)-6-hydroxy-2-azaspiro[3.3]heptane-2-carbonyl)-1-oxoisoindolin-2-yl)piperidine-2,6-dione. To a stirred solution of 2-(2,6-dioxopiperidin-3-yl)-1-oxo-3H-isoindole-5-carboxylic acid (100 mg, 0.347 mmol, 1 equiv) and 6-(4-fluorophenyl)-2-azaspiro[3.3]heptan-6-ol (107.84 mg, 0.520 mmol, 1.5 equiv) in DMF (3 mL) were added NMI (113.93 mg, 1.388 mmol, 4 equiv) and TCFH (146.0 mg, 0.520 mmol, 1.5 equiv) in portions at room temperature under nitrogen atmosphere. The resulting mixture was stirred for 3 h at room temperature under nitrogen atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(6-(4-fluorophenyl)-6-hydroxy-2-azaspiro[3.3]heptane-2-carbonyl)-1-oxoisoindolin-2-yl)piperidine-2,6-dione (7.4 mg, 4.47%) as a white solid. LC-MS (ES, m/z)=478.20 [M+1]⁺. ¹H NMR (400 MHz, DMSO-d₆) δ 11.04-10.96 (m, 1H), 7.90-7.68 (m, 3H), 7.50-7.38 (m, 2H), 7.13 (dt, J=13.0, 8.9 Hz, 2H), 5.56 (d, J=23.8 Hz, 1H), 5.13 (dt, J=13.2, 4.8 Hz, 1H), 4.56-4.32 (m, 3H), 4.25 (s, 1H), 4.19 (s, 1H), 4.02 (s, 1H), 2.99-2.85 (m, 1H), 2.72-2.56 (m, 3H), 2.43 (dd, J=19.1, 13.0 Hz, 3H), 2.02 (s, 1H).

### Example 244

### Synthesis of 3-(5-(6-(4-fluorophenyl)-6-methoxy-2-azaspiro[3.3]heptane-2-carbonyl oxoisoindolin-2-yl)piperidine-2,6-dione

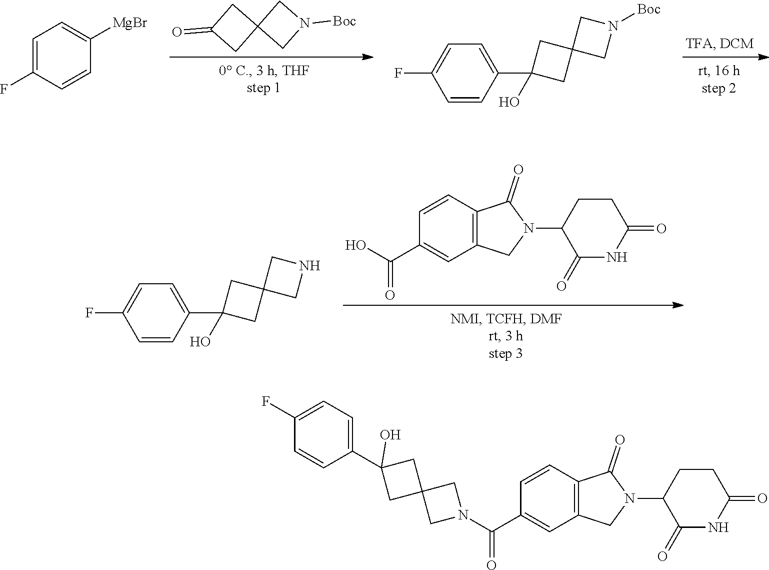

Step 1. Preparation of tert-butyl 6-(4-fluorophenyl)-6-methoxy-2-azaspiro[3.3]heptane-2-carboxylate. A solution of tert-butyl 6-(4-fluorophenyl)-6-hydroxy-2-azaspiro[3.3]heptane-2-carboxylate (240 mg, 0.781 mmol, 1 equiv) in DMF (8 mL) was treated with NaH (37.48 mg, 1.562 mmol, 2 equiv) for 30 min at 0° C. under nitrogen atmosphere followed by the addition of CH₃I (332.49 mg, 2.343 mmol, 3 equiv) dropwise at 0° C. The resulting mixture was stirred overnight at room temperature under nitrogen atmosphere. The reaction was quenched by the addition of Water (20 mL) at room temperature. The resulting mixture was extracted with EtOAc (3×30 mL). The combined organic layers were washed with brine (2×20 mL), dried over anhydrous Na₂SO₄. After filtration, the filtrate was concentrated under reduced pressure. The residue was purified by reversed-phase flash chromatography to afford tert-butyl 6-(4-fluorophenyl)-6-methoxy-2-azaspiro[3.3]heptane-2-carboxylate (220 mg, 88%) as a white solid. LC-MS (ES, m/z)=307.10 [M-tBu+ACN+1]⁺.

Step 2. Preparation of 6-(4-fluorophenyl)-6-methoxy-2-azaspiro[3.3]heptane. To a stirred solution of tert-butyl 6-(4-fluorophenyl)-6-methoxy-2-azaspiro[3.3]heptane-2-carboxylate (220 mg, 0.685 mmol, 1 equiv) in DCM (2.5 mL) were added trifluoroacetic acid (0.5 mL) dropwise at 0° C. The resulting mixture was stirred at room temperature overnight. The resulting mixture was concentrated under reduced pressure to afford 6-(4-fluorophenyl)-6-methoxy-2-azaspiro[3.3]heptane (100 mg, crude) as a brown oil. The crude product was used in the next step directly without further purification. LC-MS (ES, m/z)=222.0 [M+1]⁺.

Step 3. Preparation of 3-(5-(6-(4-fluorophenyl)-6-methoxy-2-azaspiro[3.3]heptane-2-carbonyl)-1-oxoisoindolin-2-yl)piperidine-2,6-dione. To a stirred solution of 2-(2,6-dioxopiperidin-3-yl)-1-oxo-3H-isoindole-5-carboxylic acid (50 mg, 0.173 mmol, 1 equiv) and 6-(4-fluorophenyl)-6-methoxy-2-azaspiro[3.3]heptane (57.57 mg, 0.259 mmol, 1.5 equiv) in DMF (0.5 mL) were added NMI (56.97 mg, 0.692 mmol, 4 equiv) and TCFH (73.00 mg, 0.259 mmol, 1.5 equiv) in portions at room temperature under nitrogen atmosphere. The resulting mixture was stirred overnight at room temperature under nitrogen atmosphere. The resulting mixture was diluted with water (30 mL) and extracted with EtOAc (3×15 mL). The combined organic layers were washed with brine (2×20 mL), dried over anhydrous Na₂SO₄. After filtration, the filtrate was concentrated under reduced pressure. The residue was purified by reversed-phase flash chromatography to afford 3-(5-(6-(4-fluorophenyl)-6-methoxy-2-azaspiro[3.3]heptane-2-carbonyl)-1-oxoisoindolin-2-yl)piperidine-2,6-dione (4.4 mg, 13%) as a white solid. LC-MS (ES, m/z)=492.20 [M+1]⁺. ¹H NMR (400 MHz, DMSO-d₆) δ 11.00 (s, 1H), 7.88-7.67 (m, 3H), 7.45-7.34 (m, 2H), 7.19 (dt, J=13.0, 8.9 Hz, 2H), 5.19-5.09 (m, 1H), 4.43 (h, J=16.8 Hz, 3H), 4.17 (d, J=12.5 Hz, 2H), 3.96 (s, 1H), 2.98-2.85 (m, 1H), 2.81 (d, J=6.1 Hz, 3H), 2.65 (d, J=12.5 Hz, 3H), 2.56 (d, J=10.3 Hz, 2H), 2.03 (s, 1H).

### Example 245

### Synthesis of 3-(5-(6-(3-fluoro-4-(trifluoromethyl)phenyl)-2-azaspiro[3.3]heptane-2-carbonyl)-1-oxoisoindolin-2-yl)piperidine-2,6-dione

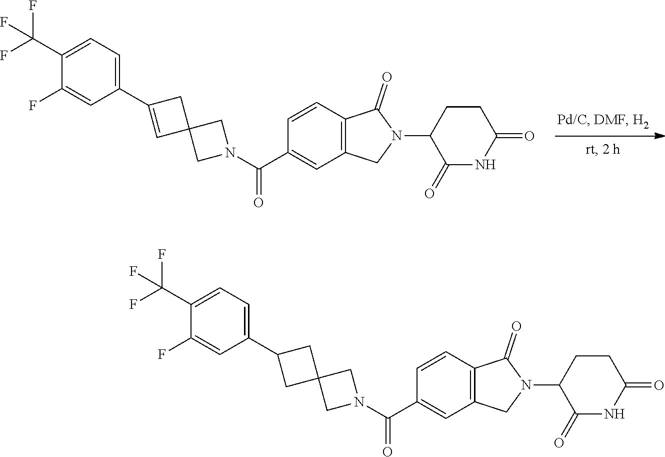 [SMILES: O=C1CCC(N2Cc3cc(C(=O)N4CC5(C=C(c6ccc(C(F)(F)F)c(F)c6)C5)C4)ccc3C2=O)C(=O)N1 ; O=C1CCC(N2Cc3cc(C(=O)N4CC5(CC(c6ccc(C(F)(F)F)c(F)c6)C5)C4)ccc3C2=O)C(=O)N1]

Step 1. Preparation of 3-(5-(6-(3-fluoro-4-(trifluoromethyl)phenyl)-2-azaspiro[3.3]heptane-2-carbonyl)-1-oxoisoindolin-2-yl)piperidine-2,6-dione. A mixture of 3-(5-{6-[3-fluoro-4-(trifluoromethyl)phenyl]-2-azaspiro[3.3]hept-5-ene-2-carbonyl}-1-oxo-3H-isoindol-2-yl)piperidine-2,6-dione (80 mg, 0.152 mmol, 1 equiv) in DMF (1.5 mL) was added Pd/C (60 mg, Palladium 10% on Carbon (wetted with ca. 55% Water)) at room temperature. The resulting mixture was stirred for 2 h at room temperature under hydrogen atmosphere. The resulting mixture was filtered through a Celite pad, the filter cake was washed with THF (3×5 mL). The filtrate was concentrated under reduced pressure. The residue was purified by reversed-phase flash chromatography to afford 3-(5-(6-(3-fluoro-4-(trifluoromethyl)phenyl)-2-azaspiro[3.3]heptane-2-carbonyl)-1-oxoisoindolin-2-yl)piperidine-2,6-dione (56 mg, 69%) as an off-white solid. LC-MS (ES, m/z)=530.20 [M+1]⁺. ¹H NMR (400 MHz, DMSO-d₆) δ 11.01 (s, 1H), 7.93-7.63 (m, 4H), 7.40 (dd, J=12.3, 5.7 Hz, 1H), 7.26 (d, J=8.1 Hz, 1H), 5.22-5.08 (m, 1H), 4.57-4.35 (m, 3H), 4.25 (d, J=5.3 Hz, 2H), 4.03 (s, 1H), 3.51 (dt, J=30.8, 8.7 Hz, 2H), 2.92 (ddd, J=18.3, 13.8, 5.4 Hz, 1H), 2.63 (dt, J=13.7, 8.2 Hz, 3H), 2.37 (dtd, J=23.8, 14.1, 11.5, 6.2 Hz, 3H), 2.14-1.94 (m, 1H).

### Example 246

### Synthesis of 3-(5-(6-(2-fluoro-4-(trifluoromethyl)phenyl)-2-azaspiro[3.3]heptane-2-carbonyl oxoisoindolin-2-yl)piperidine-2,6-dione

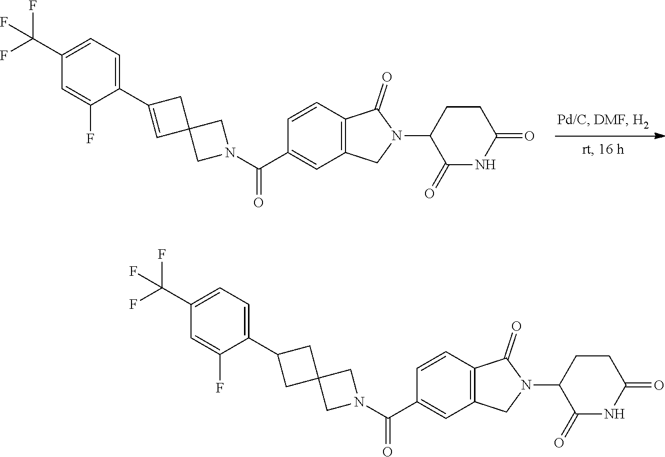 [SMILES: O=C1CCC(N2Cc3cc(C(=O)N4CC5(C=C(c6ccc(C(F)(F)F)cc6F)C5)C4)ccc3C2=O)C(=O)N1 ; O=C1CCC(N2Cc3cc(C(=O)N4CC5(CC(c6ccc(C(F)(F)F)cc6F)C5)C4)ccc3C2=O)C(=O)N1]

Step 1. Preparation of 3-(5-(6-(2-fluoro-4-(trifluoromethyl)phenyl)-2-azaspiro[3.3]heptane-2-carbonyl)-1-oxoisoindolin-2-yl)piperidine-2,6-dione. To a stirred solution of 3-(5-{6-[2-fluoro-4-(trifluoromethyl)phenyl]-2-azaspiro[3.3]hept-5-ene-2-carbonyl}-1-oxo-3H-isoindol-2-yl)piperidine-2,6-dione (30 mg, 0.057 mmol, 1 equiv) in DMF (1 mL) were added Pd/C (30 mg, Palladium 10% on Carbon (wetted with ca. 55% Water)) at room temperature. The resulting mixture was stirred overnight at room temperature under hydrogen atmosphere. The resulting mixture was filtered through a Celite pad, the filter cake was washed with THF (3×5 mL). The filtrate was concentrated under reduced pressure. The crude product (30 mg) was purified by Prep-HPLC to afford 3-(5-(6-(2-fluoro-4-(trifluoromethyl)phenyl)-2-azaspiro[3.3]heptane-2-carbonyl)-1-oxoisoindolin-2-yl)piperidine-2,6-dione (17 mg, 58%) as a white solid. LC-MS (ES, m/z)=530.20 [M+1]⁺. ¹H NMR (400 MHz, DMSO-d₆) δ 11.01 (s, 1H), 7.87 (d, J=12.3 Hz, 1H), 7.77 (p, J=8.0 Hz, 2H), 7.62-7.52 (m, 3H), 5.13 (dd, J=11.2, 6.7 Hz, 1H), 4.51 (q, J 8.2, 7.4 Hz, 2H), 4.38 (dd, J=17.5, 7.0 Hz, 1H), 4.26 (d, J=3.7 Hz, 2H), 4.02 (s, 1H), 3.62 (dt, J=27.4, 8.8 Hz, 1H), 2.94-2.85 (m, 1H), 2.69-2.56 (m, 3H), 2.38 (q, J=12.3, 11.8 Hz, 3H), 2.03 (d, J=11.4 Hz, 1H).

### Example 247

### Synthesis of 3-(5-(2-(4-fluorophenyl)-6-azaspiro[3.4]oct-1-ene-6-carbonyl)-1-oxoisoindolin-2-yl)piperidine-2,6-dione

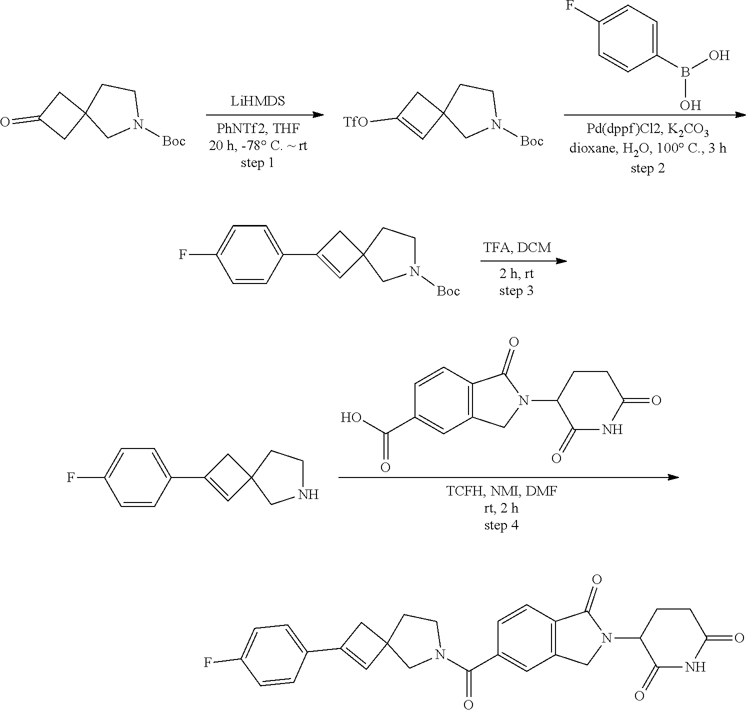

Step 1. Preparation of tert-butyl 2-(((trifluoromethyl)sulfonyl)oxy)-6-azaspiro[3.4]oct-1-ene-6-carboxylate. To a stirred solution of tert-butyl 2-oxo-6-azaspiro[3.4]octane-6-carboxylate (2.5 g, 11.097 mmol, 1 equiv) in THF (25 mL) was added LiHMDS (22.3 mL, 22.300 mmol, 2 equiv) dropwise at −78° C. under nitrogen atmosphere. The resulting mixture was stirred for 4 h at room temperature under nitrogen atmosphere. Then, the mixture was allowed to cool down to −78° C. and a solution of 1,1,1-trifluoro-N-phenyl-N-trifluoromethanesulfonylmethanesulfonamide (7.9 g, 22.114 mmol, 1.99 equiv) in THF (25 mL) was added dropwise at −78° C. The reaction mixture was warmed to room temperature and stirred overnight. The reaction was quenched with sat. NH₄Cl (aq.) at 0° C. and extracted with EtOAc (3×150 mL). The combined organic layers were washed with brine (2×100 mL), dried over anhydrous Na₂SO₄. After filtration, the filtrate was concentrated under reduced pressure. The residue was purified by reversed-phase flash chromatography to afford tert-butyl 2-(trifluoromethanesulfonyloxy)-6-azaspiro[3.4]oct-1-ene-6-carboxylate (2.6 g, 66%) as a yellow solid. LC-MS (ES, m/z)=343.05 [M-tBu+ACN+1]⁺.

Step 2. Preparation of tert-butyl 2-(4-fluorophenyl)-6-azaspiro[3.4]oct-1-ene-6-carboxylate. To a stirred mixture of tert-butyl 2-(trifluoromethanesulfonyloxy)-6-azaspiro[3.4]oct-1-ene-6-carboxylate (600 mg, 1.679 mmol, 1 equiv) and 4-fluorophenylboronic acid (352.4 mg, 2.519 mmol, 1.5 equiv) in dioxane (12 mL) and H₂O (1.2 mL) were added Pd(dppf)Cl₂ (122.9 mg, 0.168 mmol, 0.1 equiv) and K₂CO₃ (696.2 mg, 5.037 mmol, 3 equiv) at room temperature under air atmosphere. The resulting mixture was stirred for 3 h at 100° C. under nitrogen atmosphere. The mixture was allowed to cool down to room temperature. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford tert-butyl 2-(4-fluorophenyl)-6-azaspiro[3.4]oct-1-ene-6-carboxylate (450 mg, 88%) as a yellow solid. LC-MS (ES, m/z)=289.10 [M-tBu+ACN+1]⁺.

Step 3. Preparation of 2-(4-fluorophenyl)-6-azaspiro[3.4]oct-1-ene. To a stirred solution of tert-butyl 2-(4-fluorophenyl)-6-azaspiro[3.4]oct-1-ene-6-carboxylate (450 mg, 1.483 mmol, 1 equiv) in DCM (10 mL) was added TFA (2 mL, 26.926 mmol, 18.15 equiv) dropwise at 0° C. under nitrogen atmosphere. The resulting mixture was stirred for 2 h at room temperature under nitrogen atmosphere. The resulting mixture was concentrated under vacuum to afford 2-(4-fluorophenyl)-6-azaspiro[3.4]oct-1-ene (500 mg, crude) as a brown oil. The crude product was used in the next step directly without further purification. LC-MS (ES, m/z)=204.10 [M+1]⁺.

Step 4. Preparation of 3-(5-(2-(4-fluorophenyl)-6-azaspiro[3.4]oct-1-ene-6-carbonyl)-1-oxoisoindolin-2-yl)piperidine-2,6-dione. To a stirred mixture of 2-(2,6-dioxopiperidin-3-yl)-1-oxo-3H-isoindole-5-carboxylic acid (150 mg, 0.520 mmol, 1 equiv) and 2-(4-fluorophenyl)-6-azaspiro[3.4]oct-1-ene (211.6 mg, 1.041 mmol, 2 equiv) in DMF (3 mL) were added NMI (214.5 mg, 2.612 mmol, 5 equiv) and TCFH (219 mg, 0.781 mmol, 1.5 equiv) in portions at room temperature under air atmosphere. The resulting mixture was stirred for 1 h at room temperature under nitrogen atmosphere. The resulting mixture was diluted with water (50 mL) and extracted with EtOAc (3×20 mL). The combined organic layers were washed with brine (2×30 mL) and concentrated under reduced pressure. The residue was purified by reversed-phase flash chromatography to afford 3-(5-(2-(4-fluorophenyl)-6-azaspiro[3.4]oct-1-ene-6-carbonyl)-1-oxoisoindolin-2-yl)piperidine-2,6-dione (39 mg, 16%) as an off-white solid. LC-MS (ES, m/z)=474.20 [M+1]⁺. ¹H NMR (400 MHz, DMSO-d₆) δ 11.00 (d, J=7.1 Hz, 1H), 7.78 (dq, J=13.4, 8.6, 7.3 Hz, 2H), 7.66 (dd, J=11.5, 8.2 Hz, 1H), 7.43 (dtd, J=27.6, 5.7, 2.1 Hz, 2H), 7.26-7.09 (m, 2H), 6.43 (d, J=40.4 Hz, 1H), 5.14 (dt, J=12.7, 5.9 Hz, 1H), 4.51 (dt, J=17.6, 7.2 Hz, 1H), 4.38 (ddd, J=17.1, 9.3, 6.4 Hz, 1H), 3.73-3.36 (m, 4H), 3.00-2.71 (m, 2H), 2.68-2.55 (m, 2H), 2.47-2.32 (m, 1H), 2.02 (dddd, J=30.2, 25.2, 11.8, 7.8 Hz, 3H).

### Example 248

### Synthesis of 3-(5-(2-(4-fluorophenyl)-6-azaspiro[3.4]octane-6-carbonyl)-1-oxoisoindolin-2-yl)piperidine-2,6-dione

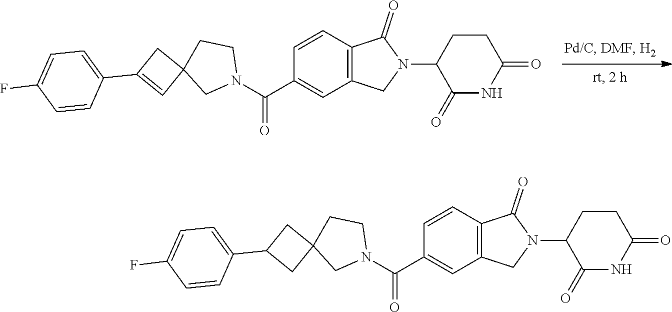 [SMILES: O=C1CCC(N2Cc3cc(C(=O)N4CCC5(C=C(c6ccc(F)cc6)C5)C4)ccc3C2=O)C(=O)N1 ; O=C1CCC(N2Cc3cc(C(=O)N4CCC5(CC(c6ccc(F)cc6)C5)C4)ccc3C2=O)C(=O)N1]

Step 1. Preparation of 3-(5-(2-(4-fluorophenyl)-6-azaspiro[3.4]octane-6-carbonyl)-1-oxoisoindolin-2-yl)piperidine-2,6-dione. To a solution of 3-{5-[6-(4-methylphenyl)-2-azaspiro[3.3]hept-5-ene-2-carbonyl]-1-oxo-3H-isoindol-2-yl}piperidine-2,6-dione (50 mg, 0.110 mmol, 1 equiv) in DMF (1 mL) was added Pd/C (25 mg, Palladium 10% on Carbon (wetted with ca. 55% Water)) under nitrogen atmosphere in a 8 mL vial. The mixture was hydrogenated at room temperature for 2 h under hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford 3-(5-(2-(4-fluorophenyl)-6-azaspiro[3.4]octane-6-carbonyl)-1-oxoisoindolin-2-yl)piperidine-2,6-dione (41 mg, 82%) as an off-white solid. LC-MS (ES, m/z)=476.20 [M+1]⁺. ¹H NMR (400 MHz, DMSO-d₆) δ 11.01 (s, 1H), 7.84-7.71 (m, 2H), 7.70-7.58 (m, 1H), 7.36-7.20 (m, 2H), 7.17-7.00 (m, 2H), 5.14 (dd, J=13.3, 5.1 Hz, 1H), 4.50 (d, J=17.5 Hz, 1H), 4.38 (d, J=17.5 Hz, 1H), 3.40-3.35 (s, 1H), 3.69-3.43 (m, 4H), 2.92 (ddd, J=17.8, 13.5, 5.3 Hz, 1H), 2.65-2.57 (m, 1H), 2.48-2.30 (m, 2H), 2.21 (ddd, J=24.4, 11.7, 8.9 Hz, 2H), 2.14-1.85 (m, 4H).

### Example 249

### Synthesis of 3-(5-(2-(3-fluorophenyl)-6-azaspiro[3.4]oct-1-ene-6-carbonyl)-1-oxoisoindolin-2-yl)piperidine-2,6-dione

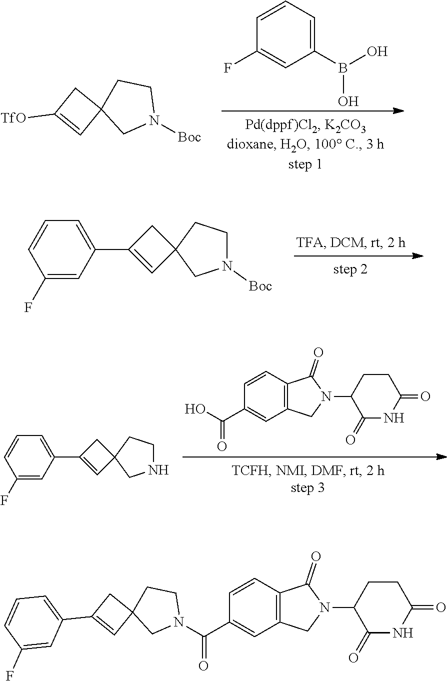 [SMILES: CC(C)(C)OC(=O)N1CCC2(C=C(OS(=O)(=O)C(F)(F)F)C2)C1 ; OB(O)c1cccc(F)c1 ; CC(C)(C)OC(=O)N1CCC2(C=C(c3cccc(F)c3)C2)C1 ; Fc1cccc(C2=CC3(CCNC3)C2)c1 ; O=C1CCC(N2Cc3cc(C(=O)O)ccc3C2=O)C(=O)N1 ; O=C1CCC(N2Cc3cc(C(=O)N4CCC5(C=C(c6cccc(F)c6)C5)C4)ccc3C2=O)C(=O)N1]

Step 1. Preparation of tert-butyl 2-(3-fluorophenyl)-6-azaspiro[3.4]oct-1-ene-6-carboxylate. A mixture of tert-butyl 2-(trifluoromethanesulfonyloxy)-6-azaspiro[3.4]oct-1-ene-6-carboxylate (500 mg, 1.399 mmol, 1 equiv), 3-fluorophenylboronic acid (391.56 mg, 2.798 mmol, 2 equiv), Pd(dppf)Cl₂ (102.38 mg, 0.140 mmol, 0.1 equiv) and K₂CO₃ (580.14 mg, 4.197 mmol, 3 equiv) in dioxane (10 mL) and H₂O (1 mL) was stirred for 3 h at 100° C. under nitrogen atmosphere. The mixture was allowed to cool down to room temperature. The resulting mixture was filtered, the filter cake was washed with dioxane (3×10 mL). The filtrate was concentrated under reduced pressure. The residue was purified by reversed-phase flash chromatography to afford tert-butyl 2-(3-fluorophenyl)-6-azaspiro[3.4]oct-1-ene-6-carboxylate (320 mg, 75%) as a brown oil. LC-MS (ES, m/z)=289.05 [M-tBu+ACN+1]⁺.

Step 2. Preparation of 2-(3-fluorophenyl)-6-azaspiro[3.4]oct-1-ene. A mixture of tert-butyl 2-(3-fluorophenyl)-6-azaspiro[3.4]oct-1-ene-6-carboxylate (320 mg, 1.055 mmol, 1 equiv) and TFA (1 mL) in DCM (5 mL) was stirred for 2 h at room temperature under air atmosphere. The resulting mixture was concentrated under vacuum. This resulted in 2-(3-fluorophenyl)-6-azaspiro[3.4]oct-1-ene (400 mg, crude) as a brown oil. The resulting mixture was used in the next step directly without further purification. The crude product was used in the next step directly without further purification. LC-MS (ES, m/z)=204.1 [M+1]⁺.

Step 3. Preparation of 3-(5-(2-(3-fluorophenyl)-6-azaspiro[3.4]oct-1-ene-6-carbonyl)-1-oxoisoindolin-2-yl)piperidine-2,6-dione. To a stirred mixture of 2-(3-fluorophenyl)-6-azaspiro[3.4]oct-1-ene (141.0 mg, 0.694 mmol, 1 equiv), 2-(2,6-dioxopiperidin-3-yl)-1-oxo-3H-isoindole-5-carboxylic acid (200 mg, 0.694 mmol, 1.00 equiv) and NMI (284.8 mg, 3.470 mmol, 5 equiv) in DMF (4 mL) was added TCFH (292.0 mg, 1.041 mmol, 1.5 equiv) in portions at 0° C. under air atmosphere. The resulting mixture was stirred for 2 h at room temperature under the air atmosphere. The resulting mixture was diluted with water (50 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by reversed-phase flash chromatography to afford 3-(5-(2-(3-fluorophenyl)-6-azaspiro[3.4]oct-1-ene-6-carbonyl)-1-oxoisoindolin-2-yl)piperidine-2,6-dione (43.2 mg, 13%) as an off-white solid. LC-MS (ES, m/z)=474.20 [M+1]⁺. ¹H NMR (400 MHz, DMSO-d₆) δ 11.00 (d, J=7.2 Hz, 1H), 7.86-7.74 (m, 2H), 7.72-7.64 (m, 1H), 7.50-7.34 (m, 1H), 7.29-7.09 (m, 3H), 6.66-6.49 (m, 1H), 5.13 (ddd, J=14.3, 6.9, 3.6 Hz, 1H), 4.64-4.26 (m, 2H), 3.80-3.40 (m, 4H), 3.01-2.81 (m, 1H), 2.74 (d, J=12.8 Hz, 1H), 2.70-2.55 (m, 2H), 2.48-2.35 (m, 1H), 2.04 (dddd, J=25.1, 19.6, 16.4, 8.4 Hz, 3H).

### Example 250

### Synthesis of 3-(5-(2-(3-fluorophenyl)-6-azaspiro[3.4]octane-6-carbonyl)-1-oxoisoindolin-2-yl)piperidine-2,6-dione

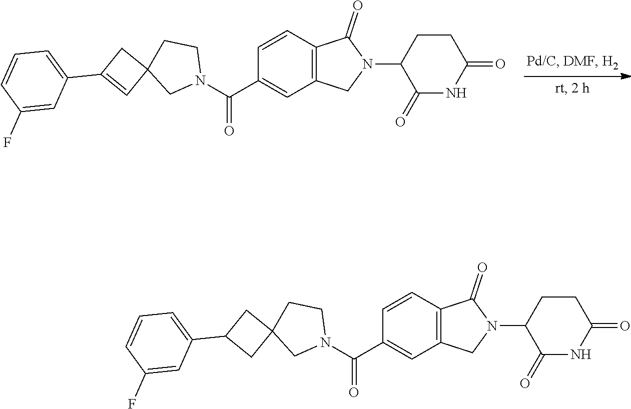 [SMILES: O=C1CCC(N2Cc3cc(C(=O)N4CCC5(C=C(c6cccc(F)c6)C5)C4)ccc3C2=O)C(=O)N1 ; O=C1CCC(N2Cc3cc(C(=O)N4CCC5(CC(c6cccc(F)c6)C5)C4)ccc3C2=O)C(=O)N1]

Step 1. Preparation of 3-(5-(2-(3-fluorophenyl)-6-azaspiro[3.4]octane-6-carbonyl)-1-oxoisoindolin-2-yl)piperidine-2,6-dione. A mixture of 3-{5-[2-(3-fluorophenyl)-6-azaspiro[3.4]oct-1-ene-6-carbonyl]-1-oxo-3H-isoindol-2-yl}piperidine-2,6-dione (80 mg, 0.169 mmol, 1 equiv) in DMF (1.5 mL) was added Pd/C (50 mg, Palladium 10% on Carbon (wetted with ca. 55% Water)) at room temperature. The resulting mixture was stirred for 2 h at room temperature under hydrogen atmosphere. The resulting mixture was filtered through a Celite pad, the filter cake was washed with THF (3×5 mL). The filtrate was concentrated under reduced pressure. The residue was purified by reversed-phase flash chromatography to afford 3-(5-(2-(3-fluorophenyl)-6-azaspiro[3.4]octane-6-carbonyl)-1-oxoisoindolin-2-yl)piperidine-2,6-dione (42 mg, 52%) as an off-white solid. LC-MS (ES, m/z)=476.25 [M+1]⁺. ¹H NMR (400 MHz, DMSO-d₆) δ 11.01 (s, 1H), 7.91-7.71 (m, 2H), 7.69-7.56 (m, 1H), 7.31 (dtd, J=30.4, 7.9, 6.1 Hz, 1H), 7.19-6.91 (m, 3H), 5.15 (dt, J=13.3, 5.3 Hz, 1H), 4.50 (d, J=17.5 Hz, 1H), 4.38 (d, J=17.5 Hz, 1H), 3.77-3.43 (m, 4H), 3.42-3.28 (m, 3H), 2.92 (ddd, J=17.2, 13.6, 5.4 Hz, 1H), 2.61 (dt, J=17.2, 3.8 Hz, 1H), 2.48-2.31 (m, 2H), 2.23 (tdd, J=11.7, 8.7, 3.5 Hz, 2H), 2.17-1.97 (m, 4H).

### Example 251

### Synthesis of 3-(1-oxo-5-(7-(trifluoromethyl)-2-azaspiro[3.5]nonane-2-carbonyl)isoindolin-2-yl)piperidine-2,6-dione

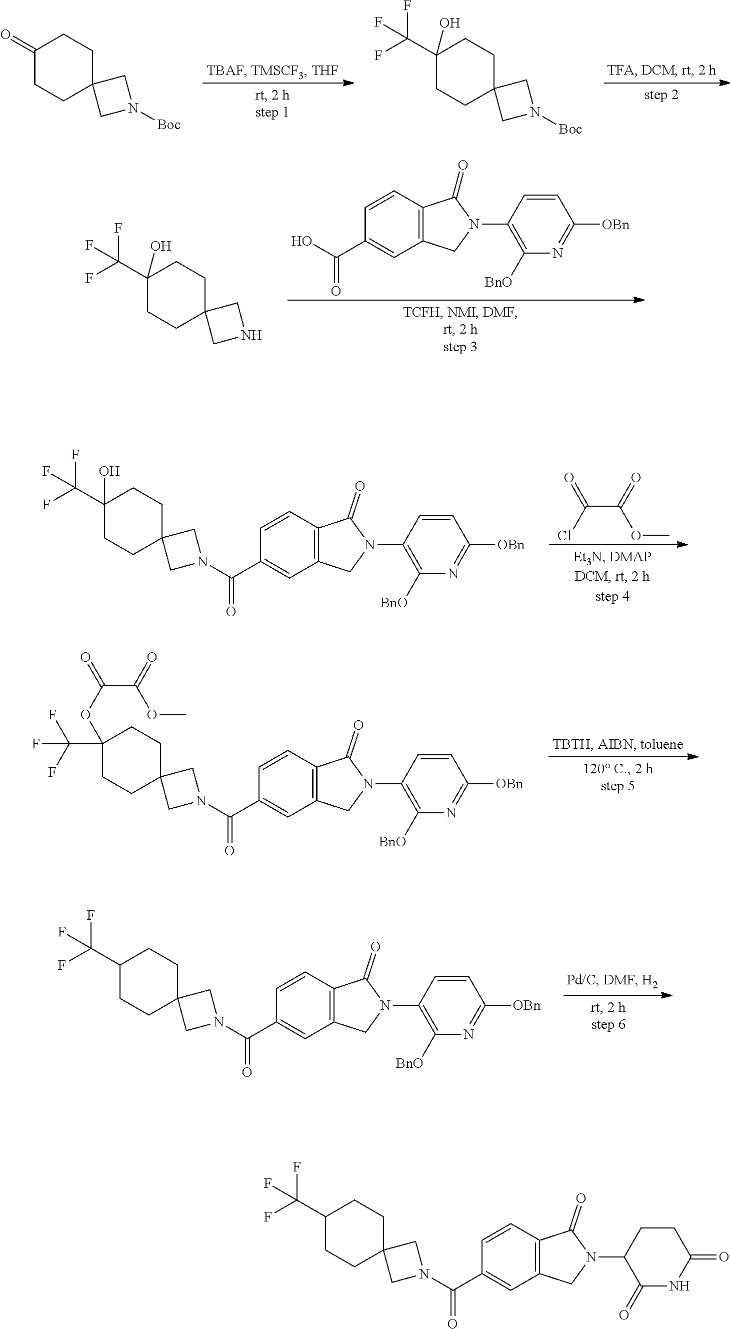

Step 1. Preparation of tert-butyl 7-hydroxy-7-(trifluoromethyl)-2-azaspiro[3.5]nonane-2-carboxylate. To a stirred mixture of tert-butyl 7-oxo-2-azaspiro[3.5]nonane-2-carboxylate (1 g, 4.179 mmol, 1 equiv) and TMSCF₃ (1.19 g, 8.358 mmol, 2 equiv) in THF (10 mL) was added TBAF (8.36 mL, 8.358 mmol, 2 equiv) at 0° C. under nitrogen atmosphere. The resulting mixture was stirred for 2 h at room temperature under nitrogen atmosphere. The resulting mixture was diluted with water and extracted with DCM (3×50 mL). The combined organic layers were washed with brine (3×100 mL), dried over anhydrous Na₂SO₄. After filtration, the filtrate was concentrated under reduced pressure. The residue was purified by reversed-phase flash chromatography to afford tert-butyl 7-hydroxy-7-(trifluoromethyl)-2-azaspiro[3.5]nonane-2-carboxylate (1.2 g, 93%) as a white solid. LC-MS (ES, m/z)=295.10 [M-tBu+ACN+1]⁺.

Step 2. Preparation of 7-(trifluoromethyl)-2-azaspiro[3.5]nonan-7-ol. To a stirred mixture of tert-butyl 7-hydroxy-7-(trifluoromethyl)-2-azaspiro[3.5]nonane-2-carboxylate (1.2 g, 3.879 mmol, 1 equiv) in DCM (24 mL) was added TFA (4.80 mL, 64.624 mmol, 16.66 equiv) dropwise at room temperature under nitrogen atmosphere. The resulting mixture was stirred for 2 h at room temperature under nitrogen atmosphere. The resulting mixture was concentrated under reduced pressure to afford 7-(trifluoromethyl)-2-azaspiro[3.5]nonan-7-ol (2 g, crude) as a white solid. LC-MS (ES, m/z)=210.0 [M+1]⁺.

Step 3. Preparation of 2-(2,6-bis(benzyloxy)pyridin-3-yl)-5-(7-hydroxy-7-(trifluoromethyl)-2-azaspiro[3.5]nonane-2-carbonyl)isoindolin-1-one. To a stirred mixture of 7-(trifluoromethyl)-2-azaspiro[3.5]nonan-7-ol (448.5 mg, 2.144 mmol, 2 equiv) and 2-[2,6-bis(benzyloxy)pyridin-3-yl]-1-oxo-3H-isoindole-5-carboxylic acid (500 mg, 1.072 mmol, 1.00 equiv) in DMF (10 mL) was added TCFH (451.1 mg, 1.608 mmol, 1.5 equiv) and NMI (352.0 mg, 4.288 mmol, 4 equiv) at room temperature under nitrogen atmosphere. The resulting mixture was stirred for 2 h at room temperature under nitrogen atmosphere. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(7-hydroxy-7-(trifluoromethyl)-2-azaspiro[3.5]nonane-2-carbonyl)isoindolin-1-one (580 mg, 71%) as a white solid. LC-MS (ES, m/z)=658.35 [M+1]⁺.

Step 4. Preparation of 2-(2-(2,6-bis(benzyloxy)pyridin-3-yl)-1-oxoisoindoline-5-carbonyl)-7-(trifluoromethyl)-2-azaspiro[3.5]nonan-7-yl methyl oxalate. A mixture of 2-(2,6-bis(benzyloxy)pyridin-3-yl)-5-(7-hydroxy-7-(trifluoromethyl)-2-azaspiro[3.5]nonane-2-carbonyl)isoindolin-1-one (600 mg, 0.912 mmol, 1 equiv), Et₃N (277.0 mg, 2.736 mmol, 3 equiv) and DMAP (11.2 mg, 0.091 mmol, 0.1 equiv) in DCM (9 mL) was added methyl oxalochloridate (223.5 mg, 1.824 mmol, 2 equiv) dropwise at 0° C. The resulting mixture was stirred for 2 h at room temperature under nitrogen atmosphere. The reaction was quenched with Water at 0° C. The resulting mixture was extracted with CH₂Cl₁₋₂ (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford 2-(2-(2,6-bis(benzyloxy)pyridin-3-yl)-1-oxoisoindoline-5-carbonyl)-7-(trifluoromethyl)-2-azaspiro[3.5]nonan-7-yl methyl oxalate (580 mg, 85%) as a yellow oil. LC-MS (ES, m/z)=744.30 [M+1]⁺.

Step 5. Preparation of 2-(2,6-bis(benzyloxy)pyridin-3-yl)-5-(7-(trifluoromethyl)-2-azaspiro[3.5]nonane-2-carbonyl)isoindolin-1-one. A mixture of 2-{2-[2,6-bis(benzyloxy)pyridin-3-yl]-1-oxo-3H-isoindole-5-carbonyl}-7-(trifluoromethyl)-2-azaspiro[3.5]nonan-7-yl methyl oxalate (300 mg, 0.403 mmol, 1 equiv), TBTH (587.0 mg, 2.015 mmol, 5 equiv) and AIBN (66.2 mg, 0.403 mmol, 1 equiv) in toluene (4.5 mL) was stirred for 2 h at 120° C. under nitrogen atmosphere. The mixture was allowed to cool down to room temperature. The resulting mixture was diluted with water (50 mL). The resulting mixture was extracted with EtOAc (3×30 mL). The combined organic layers were washed with brine (3×50 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-5-(7-(trifluoromethyl)-2-azaspiro[3.5]nonane-2-carbonyl)isoindolin-1-one (153 mg, 59%) as a yellow oil. LC-MS (ES, m/z)=642.30 [M+1]⁺.

Step 6. Preparation of 3-(1-oxo-5-(7-(trifluoromethyl)-2-azaspiro[3.5]nonane-2-carbonyl)isoindolin-2-yl)piperidine-2,6-dione. A mixture of 2-(2,6-bis(benzyloxy)pyridin-3-yl)-5-(7-(trifluoromethyl)-2-azaspiro[3.5]nonane-2-carbonyl)isoindolin-1-one (40 mg, 0.062 mmol, 1 equiv) and AcOH (37.4 mg, 0.620 mmol, 10 equiv) in DMF (0.8 mL) was added Pd/C (40 mg, Palladium 10% on Carbon (wetted with ca. 55% Water)) at room temperature. The resulting mixture was stirred for 2 h at room temperature under hydrogen (10 atm) atmosphere. The resulting mixture was filtered through a Celite pad, the filter cake was washed with THF (3×5 mL). The filtrate was concentrated under reduced pressure. The residue was purified by reversed-phase flash chromatography to afford 3-(1-oxo-5-(7-(trifluoromethyl)-2-azaspiro[3.5]nonane-2-carbonyl)isoindolin-2-yl)piperidine-2,6-dione (15 mg, 50%) as an off-white solid. LC-MS (ES, m/z)=464.20 [M+1]⁺. ¹H NMR (400 MHz, DMSO-d₆) δ 11.00 (s, 1H), 7.87 (d, J=7.7 Hz, 1H), 7.77 (d, J=8.3 Hz, 2H), 5.14 (dd, J=13.4, 5.0 Hz, 1H), 4.51 (dd, J=17.5, 5.2 Hz, 1H), 4.38 (dd, J=17.5, 4.6 Hz, 1H), 4.05 (s, 1H), 3.95 (s, 1H), 3.80 (s, 1H), 3.72 (s, 1H), 2.92 (ddd, J=18.2, 13.5, 5.4 Hz, 1H), 2.72-2.55 (m, 1H), 2.41 (qd, J=13.1, 4.4 Hz, 1H), 2.23 (d, J=10.6 Hz, 1H), 2.00 (t, J=14.8 Hz, 3H), 1.76 (s, 2H), 1.50 (td, J=14.8, 11.2 Hz, 2H), 1.25 (dq, J=37.2, 12.6 Hz, 2H).

### Example 252

### Synthesis of 3-(6-(6-(trifluoromethyl)-2-azaspiro[3.4]octane-2-carbonyl benzo[d]oxazol-2-yl)piperidine-2,6-dione

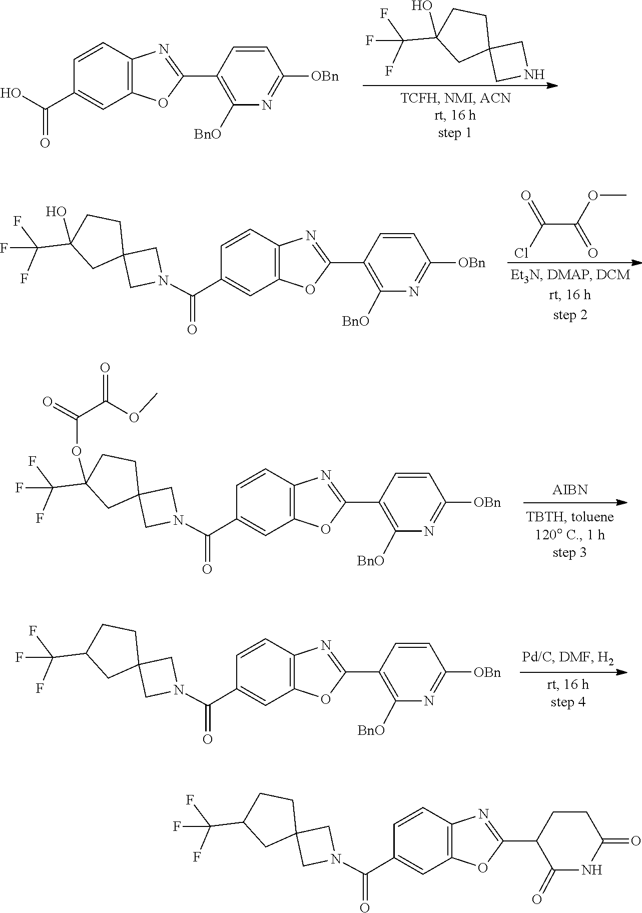

Step 1. Preparation of (2-(2,6-bis(benzyloxy)pyridin-3-yl)benzo[d]oxazol-6-yl)(6-hydroxy-6-(trifluoromethyl)-2-azaspiro[3.4]octan-2-yl)methanone. To a stirred mixture of 2-[2,6-bis(benzyloxy)pyridin-3-yl]-1,3-benzoxazole-6-carboxylic acid (400 mg, 0.884 mmol, 1 equiv) and 6-(trifluoromethyl)-2-azaspiro[3.4]octan-6-ol (396.87 mg, 2.033 mmol, 2.3 equiv) in ACN (10 mL) were added NMI (217.76 mg, 2.652 mmol, 3 equiv) and TCFH (372.07 mg, 1.326 mmol, 1.5 equiv) in portions at room temperature. The resulting mixture was stirred overnight at room temperature. The resulting mixture was diluted with water (50 mL) and extracted with EtOAc (3×30 mL). The combined organic layers were washed with brine (2×30 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(6-hydroxy-6-(trifluoromethyl)-2-azaspiro[3.4]octan-2-yl)methanone (430 mg, 77%) as a white solid. LC-MS (ES, m/z)=630.25 [M+1]⁺.

Step 2. Preparation of 2-(2-(2,6-bis(benzyloxy)pyridin-3-yl)benzo[d]oxazole-6-carbonyl)-6-(trifluoromethyl)-2-azaspiro[3.4]octan-6-yl methyl oxalate. To a stirred mixture of (2-(2,6-bis(benzyloxy)pyridin-3-yl)benzo[d]oxazol-6-yl)(6-hydroxy-6-(trifluoromethyl)-2-azaspiro[3.4]octan-2-yl)methanone (200 mg, 0.318 mmol, 1 equiv), Et₃N (96.43 mg, 0.954 mmol, 3 equiv) and DMAP (3.8 mg, 0.0318 mmol, 0.1 equiv) in DCM (5 mL) were added methyl oxalochloridate (77.82 mg, 0.636 mmol, 2 equiv) dropwise at 0° C. The resulting mixture was stirred overnight at room temperature. The resulting mixture was diluted with water (50 mL) and extracted with DCM (3×30 mL). The combined organic layers were washed with brine (2×30 mL), dried over anhydrous Na₂SO₄. After filtration, the filtrate was concentrated under reduced pressure. The residue was purified by reversed-phase flash chromatography to afford 2 (2-(2,6-bis(benzyloxy)pyridin-3-yl)benzo[d]oxazole-6-carbonyl)-6-(trifluoromethyl)-2-azaspiro[3.4]octan-6-yl methyl oxalate (150 mg, 59%) as a white solid. LC-MS (ES, m/z)=716.45 [M+1].

Step 3. Preparation of (2-(2,6-bis(benzyloxy)pyridin-3-yl)benzo[d]oxazol-6-yl)(6-(trifluoromethyl)-2-azaspiro[3.4]octan-2-yl)methanone. To a stirred solution of 2-(2-(2,6-bis(benzyloxy)pyridin-3-yl)benzo[d]oxazole-6-carbonyl)-6-(trifluoromethyl)-2-azaspiro[3.4]octan-6-yl methyl oxalate (130 mg, 0.182 mmol, 1 equiv) in Toluene (3 mL) were added AIBN (29.83 mg, 0.182 mmol, 1 equiv) and TBTH (264.35 mg, 0.910 mmol, 5 equiv) at room temperature under nitrogen atmosphere. The resulting mixture was stirred for 1 h at 120° C. The resulting mixture was diluted with water (50 mL) and extracted with EtOAc (3×30 mL). The combined organic layers were washed with brine (2×30 mL), dried over anhydrous Na₂SO₄. The residue was purified by reversed-phase flash chromatography to afford (2-(2,6-bis(benzyloxy)pyridin-3-yl)benzo[d]oxazol-6-yl)(6-(trifluoromethyl)-2-azaspiro[3.4]octan-2-yl)methanone (80 mg, 65%) as a brown oil. LC-MS (ES, m/z)=614.40 [M+1].

Step 4. Preparation of 3-(6-(6-(trifluoromethyl)-2-azaspiro[3.4]octane-2-carbonyl)benzo[d]oxazol-2-yl)piperidine-2,6-dione. To a stirred solution of (2-(2,6-bis(benzyloxy)pyridin-3-yl)benzo[d]oxazol-6-yl)(6-(trifluoromethyl)-2-azaspiro[3.4]octan-2-yl)methanone (40 mg, 0.065 mmol, 1 equiv) in DMF (1 mL) were added Pd/C (40 mg, Palladium 10% on Carbon (wetted with ca. 55% Water)) at room temperature under hydrogen atmosphere. The resulting mixture was stirred overnight at room temperature under hydrogen atmosphere. The resulting mixture was filtered through a Celite pad, the filter cake was washed with THF (3×5 mL). The filtrate was concentrated under reduced pressure. The residue was purified by reversed-phase flash chromatography to afford 3-(6-(6-(trifluoromethyl)-2-azaspiro[3.4]octane-2-carbonyl)benzo[d]oxazol-2-yl)piperidine-2,6-dione (2.2 mg, 7.08%) as an off-white solid. LC-MS (ES, m/z)=436.10 [M+1]⁺. ¹H NMR (400 MHz, DMSO-d₆) δ 7.06-6.97 (m, 1H), 6.90-6.81 (m, 1H), 6.80-6.74 (m, 1H), 3.64 (s, 2H), 3.33 (dd, J=24.9, 10.4 Hz, 2H), 3.22-3.06 (m, 2H), 2.83-2.71 (m, 1H), 1.94-1.82 (m, 2H), 1.77-1.56 (m, 1H), 1.54-1.42 (m, 1H), 1.34-1.22 (m, 1H), 1.10-1.00 (m, 4H), 0.94-0.76 (m, 1H).

### Example 253

### Synthesis of (2-(2,6-dioxopiperidin-3-yl)benzo[d]oxazol-5-yl)methyl (6,6-difluorospiro[3.3]heptan-2-yl)carbamate

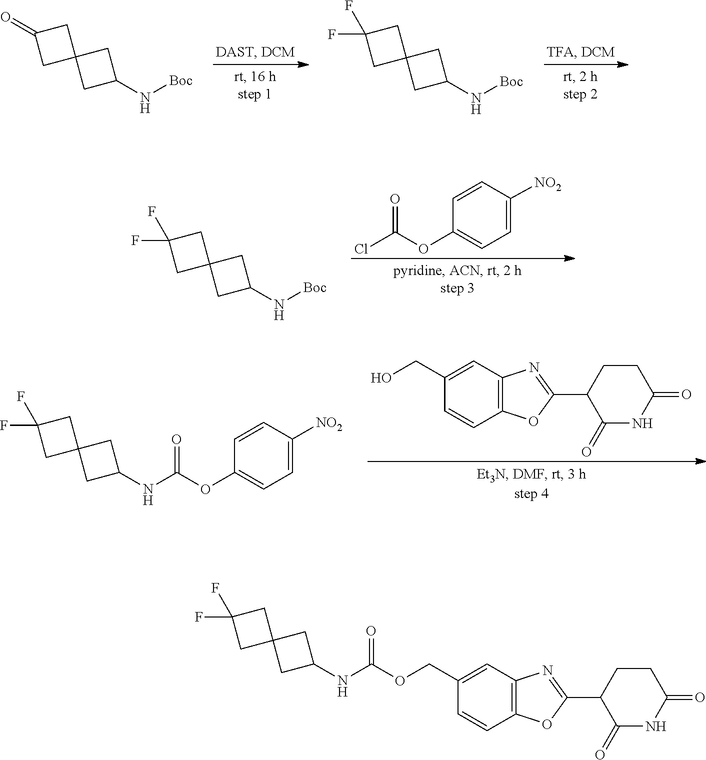

Step 1. Preparation of tert-butyl (6,6-difluorospiro[3.3]heptan-2-yl)carbamate. To a stirred solution of tert-butyl N-{6-oxospiro[3.3]heptan-2-yl}carbamate (1 g, 4.439 mmol, 1 equiv) in DCM (15 mL) was added DAST (1.76 mL, 13.317 mmol, 3 equiv) dropwise at 0° C. under nitrogen atmosphere. The resulting mixture was stirred overnight at room temperature under nitrogen atmosphere. The reaction was quenched with sat. NH₄Cl (aq.) at 0° C. and extracted with CH₂Cl₂ (3×50 mL). The combined organic layers were washed with brine (2×30 mL), dried over anhydrous Na₂SO₄. After filtration, the filtrate was concentrated under reduced pressure. The residue was purified by reversed-phase flash chromatography to afford tert-butyl (6,6-difluorospiro[3.3]heptan-2-yl)carbamate (1 g, 91%) as a yellow solid. LC-MS (ES, m/z)=233.10 [M+1]⁺.

Step 2. Preparation of 6,6-difluorospiro[3.3]heptan-2-amine. To a stirred solution of tert-butyl (6,6-difluorospiro[3.3]heptan-2-yl)carbamate (1 g, 4.044 mmol, 1 equiv) in DCM (20 mL) was added TFA (4 mL) dropwise at 0° C. under nitrogen atmosphere. The resulting mixture was stirred for 2 h at room temperature under nitrogen atmosphere. The resulting mixture was concentrated under vacuum to afford 6,6-difluorospiro[3.3]heptan-2-amine (1 g, crude) as a brown semi-solid. The crude product was used in the next step directly without further purification. LC-MS (ES, m/z)=148.10 [M+1]⁺.

Step 3. Preparation of 4-nitrophenyl (6,6-difluorospiro[3.3]heptan-2-yl)carbamate. To a stirred mixture of 6,6-difluorospiro[3.3]heptan-2-amine (500 mg, 3.397 mmol, 1 equiv) and pyridine (1.37 mL, 16.939 mmol, 4.99 equiv) in ACN (5 mL) was added 4-nitrophenyl carbonochloridate (1.37 g, 6.794 mmol, 2 equiv) in ACN (5 mL) dropwise at 0° C. under nitrogen atmosphere. The resulting mixture was stirred for 2 h at room temperature under nitrogen atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 4-nitrophenyl (6,6-difluorospiro[3.3]heptan-2-yl)carbamate (500 mg, 47%) as a yellow solid. LC-MS (ES, m/z)=354.25 [M+ACN+1]⁺.

Step 4. Preparation of (2-(2,6-dioxopiperidin-3-yl)benzo[d]oxazol-5-yl)methyl (6,6-difluorospiro[3.3]heptan-2-yl)carbamate. To a stirred mixture of 3-[5-(hydroxymethyl)-1,3-benzoxazol-2-yl]piperidine-2,6-dione (50 mg, 0.192 mmol, 1.00 equiv), 4-nitrophenyl (6,6-difluorospiro[3.3]heptan-2-yl)carbamate (180 mg, 0.576 mmol, 3.00 equiv) and DMAP (11.8 mg, 0.097 mmol, 0.50 equiv) in DMF (1 mL) was added Et₃N (0.13 mL, 0.935 mmol, 4.87 equiv) dropwise at room temperature under nitrogen atmosphere. The resulting mixture was stirred for 3 h at room temperature under nitrogen atmosphere. The resulting mixture was diluted with water (50 mL) and extracted with EtOAc (3×30 mL). The combined organic layers were washed with brine (2×30 mL), dried over anhydrous Na₂SO₄. After filtration, the filtrate was concentrated under reduced pressure. The residue was purified by reversed-phase flash chromatography to afford crude product (35 mg). The crude product was purified by Prep-HPLC to afford (2-(2,6-dioxopiperidin-3-yl)benzo[d]oxazol-5-yl)methyl (6,6-difluorospiro[3.3]heptan-2-yl)carbamate (6.0 mg, 7.21%) as an off-white solid. LC-MS (ES, m/z)=432.10 [M−1]⁻. ¹H NMR (400 MHz, DMSO-d₆) δ 11.12 (s, 1H), 7.76-7.69 (m, 2H), 7.58 (d, J=7.8 Hz, 1H), 7.40 (dd, J=8.4, 1.7 Hz, 1H), 5.10 (s, 2H), 4.51 (dd, J=11.3, 5.0 Hz, 1H), 3.92 (h, J=8.0 Hz, 1H), 2.77-2.59 (m, 4H), 2.55 (d, J=2.4 Hz, 1H), 2.42 (dtd, J=12.9, 11.2, 4.6 Hz, 2H), 2.32 (dddd, J=12.7, 9.8, 6.3, 3.8 Hz, 3H), 2.05 (td, J=9.0, 3.0 Hz, 2H).

### Example 254

### Synthesis of N-(6-(2,4-difluorophenyl)spiro[3,3]hept-5-en-2-yl)-2-(2,6-dioxopiperidin-3-yl)-6-fluoro-fluoro-1-oxoisoindoline-5-carboxamide

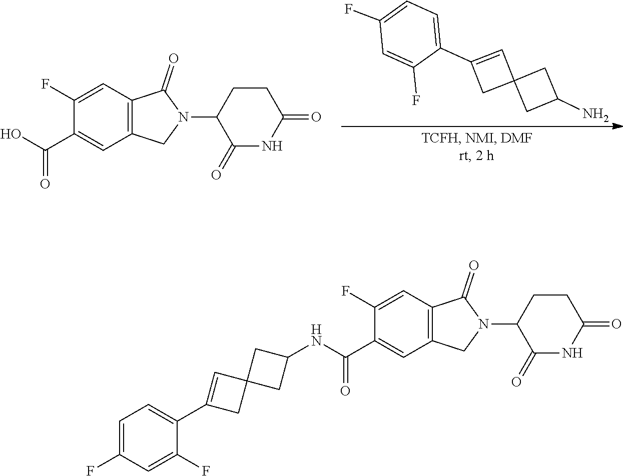 [SMILES: O=C1CCC(N2Cc3cc(C(=O)O)c(F)cc3C2=O)C(=O)N1 ; NC1CC2(C=C(c3ccc(F)cc3F)C2)C1 ; O=C1CCC(N2Cc3cc(C(=O)NC4CC5(C=C(c6ccc(F)cc6F)C5)C4)c(F)cc3C2=O)C(=O)N1]

Step 1. Preparation of N-(6-(2,4-difluorophenyl)spiro[3.3]hept-5-en-2-yl)-2-(2,6-dioxopiperidin-3-yl)-6-fluoro-1-oxoisoindoline-5-carboxamide. To a stirred mixture of 2-(2,6-dioxopiperidin-3-yl)-6-fluoro-1-oxo-3H-isoindole-5-carboxylic acid (50 mg, 0.163 mmol, 1.00 equiv) and 6-(2,4-difluorophenyl)spiro[3.3]hept-5-en-2-amine (36.1 mg, 0.163 mmol, 1.00 equiv) in DMF (1 mL) was added NMI (67.0 mg, 0.816 mmol, 5.00 equiv) and TCFH (68.7 mg, 0.245 mmol, 1.50 equiv) in portions at room temperature under nitrogen atmosphere. The resulting mixture was stirred for 1 h at room temperature under nitrogen atmosphere. The resulting mixture was diluted with water (30 mL) and extracted with EtOAc (3×20 mL). The combined organic layers were washed with brine (2×20 mL) and concentrated under reduced pressure. The residue was purified by reversed-phase flash chromatography to afford product (80 mg). The crude product (35 mg) was purified by Prep-HPLC to afford N-(6-(2,4-difluorophenyl)spiro[3.3]hept-5-en-2-yl)-2-(2,6-dioxopiperidin-3-yl)-6-fluoro-1-oxoisoindoline-5-carboxamide (14 mg, 17%) as an off-white solid. LC-MS (ES, m/z)=510.10 [M+1]⁺. ¹H NMR (400 MHz, DMSO-d₆) δ 11.01 (s, 1H), 8.80 (dd, J=7.5, 3.3 Hz, 1H), 7.80 (d, J=5.8 Hz, 1H), 7.61 (dd, J=8.8, 1.8 Hz, 1H), 7.41-7.22 (m, 2H), 7.09 (tdd, J=8.1, 4.9, 2.5 Hz, 1H), 6.34 (d, J=3.1 Hz, 1H), 5.14 (dd, J=13.3, 5.1 Hz, 1H), 4.52-4.31 (m, 3H), 2.99-2.77 (m, 3H), 2.65-2.51 (m, 2H), 2.48-2.26 (m, 4H), 2.08-1.99 (m, 1H).

### Example 255

### Synthesis of N-(6-(2,4-difluorophenyl)spiro[3.3]heptan-2-yl)-2-(2,6-dioxopiperidin-3-yl fluoro-1-oxoisoindoline-5-carboxamide

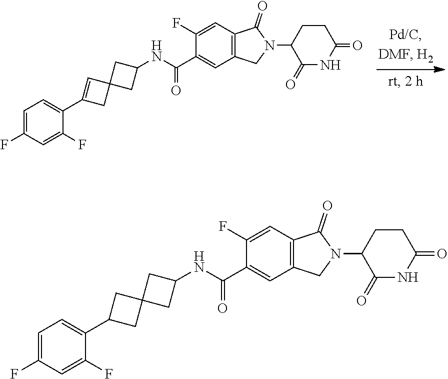 [SMILES: O=C1CCC(N2Cc3cc(C(=O)NC4CC5(C=C(c6ccc(F)cc6F)C5)C4)c(F)cc3C2=O)C(=O)N1 ; O=C1CCC(N2Cc3cc(C(=O)NC4CC5(C4)CC(c4ccc(F)cc4F)C5)c(F)cc3C2=O)C(=O)N1]

Step 1. Preparation of N-(6-(2,4-difluorophenyl)spiro[3.3]heptan-2-yl)-2-(2,6-dioxopiperidin-3-yl)-6-fluoro-1-oxoisoindoline-5-carboxamide. To a solution of N-[6-(2,4-difluorophenyl)spiro[3.3]hept-5-en-2-yl]-2-(2,6-dioxopiperidin-3-yl)-6-fluoro-1-oxo-3H-isoindole-5-carboxamide (35 mg, 0.069 mmol, 1 equiv) in DMF (0.5 mL) was added Pd/C (15 mg, Palladium 10% on Carbon (wetted with ca. 55% Water)) under nitrogen atmosphere in a 8 mL vial. The mixture was hydrogenated at room temperature for 2 h under hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford N-(6-(2,4-difluorophenyl)spiro[3.3]heptan-2-yl)-2-(2,6-dioxopiperidin-3-yl)-6-fluoro-1-oxoisoindoline-5-carboxamide (22 mg, 62%) as an off-white solid. LC-MS (ES, m/z)=512.10 [M+1]⁺. ¹H NMR (400 MHz, DMSO-d₆) δ 11.01 (s, 1H), 8.74 (d, J=7.4 Hz, 1H), 7.78 (d, J=5.8 Hz, 1H), 7.60 (d, J=8.8 Hz, 1H), 7.35 (td, J=8.7, 6.6 Hz, 1H), 7.13 (ddd, J=10.6, 9.3, 2.6 Hz, 1H), 7.03 (td, J=8.5, 2.7 Hz, 1H), 5.13 (dd, J=13.3, 5.1 Hz, 1H), 4.47 (d, J=17.4 Hz, 1H), 4.39-4.24 (m, 2H), 3.52 (p, J=8.9 Hz, 1H), 2.91 (ddd, J=17.2, 13.5, 5.4 Hz, 1H), 2.66-2.53 (m, 2H), 2.48-2.23 (m, 4H), 2.21-2.09 (m, 3H), 2.08-1.97 (m, 2H).

### Example 256

### Synthesis of 2-(2,6-dioxopiperidin-3-yl)-1-oxo-N-(6-(4-(trifluoromethyl)phenyl)spiro[3.3]hept-5-en-2-yl)isoindoline-5-carboxamide

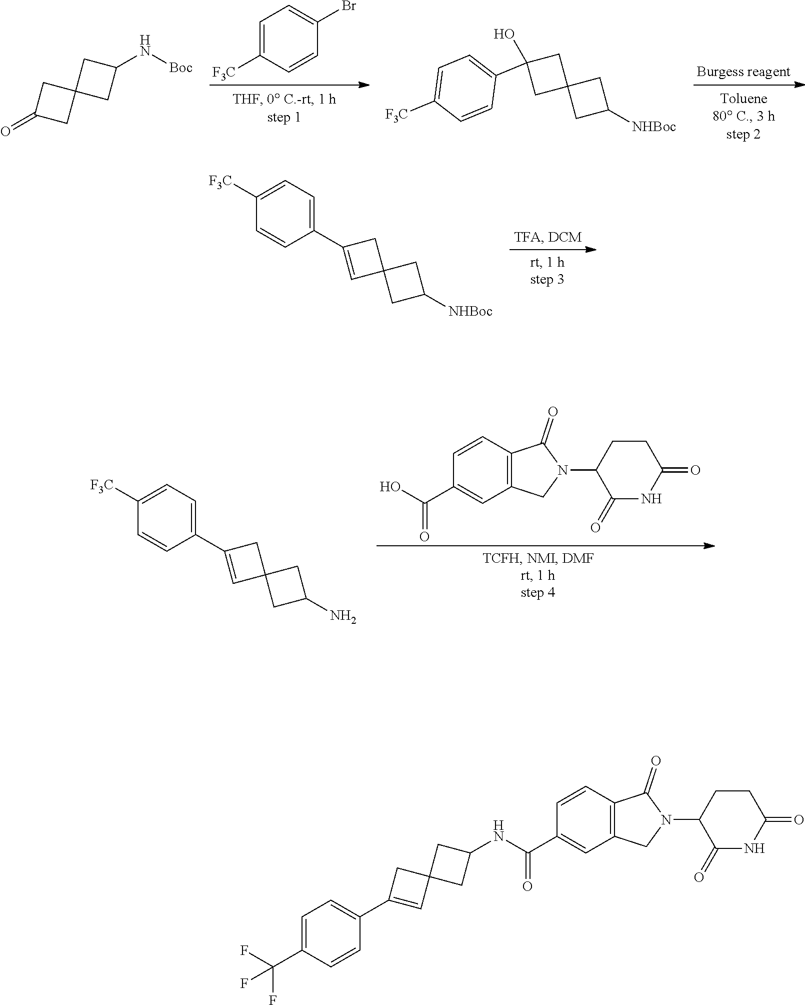

Step 1. Preparation of tert-butyl (6-hydroxy-6-(4-(trifluoromethyl)phenyl)spiro[3.3]heptan-2-yl)carbamate. To a stirred solution of tert-butyl N-{6-oxospiro[3.3]heptan-2-yl}carbamate (500 mg, 2.219 mmol, 1 equiv) in THF (10 mL) was added bromo[4-(trifluoromethyl)phenyl]magnesium (5.55 mL, 3.329 mmol, 1.5 equiv) (0.6 M in Et₂O) dropwise at 0° C. under nitrogen atmosphere. The resulting mixture was stirred for 1 h at room temperature under nitrogen atmosphere. The reaction was quenched with sat. NH₄Cl (aq.) at 0° C. and extracted with EtOAc (3×50 mL). The combined organic layers were washed with brine (2×30 mL), dried over anhydrous Na₂SO₄. After filtration, the filtrate was concentrated under reduced pressure. The residue was purified by reversed-phase flash chromatography to afford tert-butyl (6-hydroxy-6-(4-(trifluoromethyl)phenyl)spiro[3.3]heptan-2-yl)carbamate (400 mg, 49%) as an off-white solid. LC-MS (ES, m/z)=357.10 [M-tBu+ACN+1]⁺.

Step 2. Preparation of tert-butyl (6-(4-(trifluoromethyl)phenyl)spiro[3.3]hept-5-en-2-yl)carbamate. A mixture of tert-butyl (6-hydroxy-6-(4-(trifluoromethyl)phenyl)spiro[3.3]heptan-2-yl)carbamate (300 mg, 0.808 mmol, 1 equiv) and Burgess reagent (481.22 mg, 2.020 mmol, 2.50 equiv) in Toluene (6 mL) was stirred for 3 h at 80° C. under nitrogen atmosphere. The mixture was allowed to cool down to room temperature. The resulting mixture was concentrated under vacuum. The residue was purified by reversed-phase flash chromatography to afford tert-butyl (6-(4-(trifluoromethyl)phenyl)spiro[3.3]hept-5-en-2-yl)carbamate (120 mg, 42%) as an off-white solid. LC-MS (ES, m/z)=339.10 [M-tBu+ACN+1]⁺.

Step 3. Preparation of 6-(4-(trifluoromethyl)phenyl)spiro[3.3]hept-5-en-2-amine. To a stirred solution of tert-butyl (6-(4-(trifluoromethyl)phenyl)spiro[3.3]hept-5-en-2-yl)carbamate (120 mg, 0.340 mmol, 1 equiv) in DCM (3 mL) was added TFA (0.6 mL) dropwise at 0° C. under nitrogen atmosphere. The resulting mixture was stirred for 1 h at room temperature under nitrogen atmosphere. The resulting mixture was concentrated under vacuum to afford 6-[4-(trifluoromethyl)phenyl]spiro[3.3]hept-5-en-2-amine (150 mg, crude) as a brown oil. The crude product was used in the next step directly without further purification. LC-MS (ES, m/z)=295.10 [M+ACN+1]⁺.

Step 4. Preparation of 2-(2,6-dioxopiperidin-3-yl)-1-oxo-N-(6-(4-(trifluoromethyl)phenyl)spiro[3.3]hept-5-en-2-yl)isoindoline-5-carboxamide. To a stirred mixture of 2-(2,6-dioxopiperidin-3-yl)-1-oxo-3H-isoindole-5-carboxylic acid (100 mg, 0.347 mmol, 1.00 equiv) and 6-[4-(trifluoromethyl)phenyl]spiro[3.3]hept-5-en-2-amine (175.7 mg, 0.694 mmol, 2.00 equiv) in DMF (2 mL) was added NMI (143 mg, 1.742 mmol, 5.02 equiv) and TCFH (146.0 mg, 0.520 mmol, 1.50 equiv) in portions at room temperature under nitrogen atmosphere. The resulting mixture was stirred for 1 h at room temperature under nitrogen atmosphere. The resulting mixture was diluted with water (50 mL) and extracted with EtOAc (3×30 mL). The combined organic layers were washed with brine (2×50 mL), dried over anhydrous NaSO₄. After filtration, the filtrate was concentrated under reduced pressure. The residue was purified by reversed-phase flash chromatography to afford crude product (100 mg). The crude product was purified by Prep-HPLC to afford 2-(2,6-dioxopiperidin-3-yl)-1-oxo-N-(6-(4-(trifluoromethyl)phenyl)spiro[3.3]hept-5-en-2-yl)isoindoline-5-carboxamide (50 mg, 28%) as an off-white solid. LC-MS (ES, m/z)=524.20 [M+1]⁺. ¹H NMR (400 MHz, DMSO-d₆) δ 11.01 (s, 1H), 8.87 (dd, J=7.4, 4.3 Hz, 1H), 8.08 (s, 1H), 8.02-7.96 (m, 1H), 7.82 (d, J=7.9 Hz, 1H), 7.75-7.67 (m, 2H), 7.57 (t, J=8.4 Hz, 2H), 6.64 (s, 1H), 5.15 (dd, J=13.3, 5.1 Hz, 1H), 4.52 (dt, J=12.1, 7.6 Hz, 2H), 4.40 (d, J=17.5 Hz, 1H), 2.99-2.77 (m, 3H), 2.69-2.53 (m, 2H), 2.48-2.34 (m, 4H), 2.04 (ddd, J=9.9, 8.1, 3.9 Hz, 1H), 1.24 (s, 1H).

### Example 257

### Synthesis of 2-(2,6-dioxopiperidin-3-yl)-N-(6-(2-fluoro-4-(trifluoromethyl)phenyl)spiro[3.3]hept-5-en-2-yl)-1-oxoisoindoline-5-carboxamide

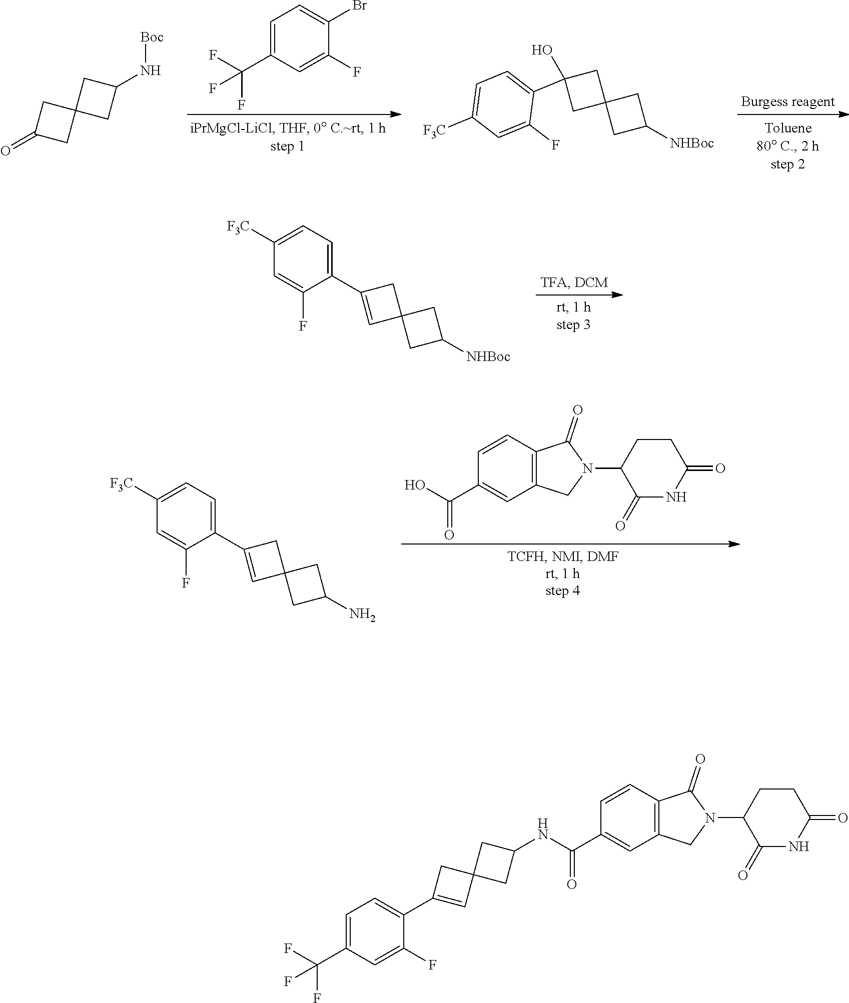

Step 1. Preparation of tert-butyl (6-(2-fluoro-4-(trifluoromethyl)phenyl)-6-hydroxyspiro[3.3]heptan-2-yl)carbamate. To a stirred solution of 1-bromo-2-fluoro-4-(trifluoromethyl)benzene (809.0 mg, 3.329 mmol, 1.50 equiv) in THF (5 mL) was added Isopropylmagnesium chloride-Lithium chloride complex (1.3M in THF) (3.41 mL, 2 equiv) dropwise at 0° C. under nitrogen atmosphere. The resulting mixture was stirred for 1 h at room temperature under nitrogen atmosphere. To the above mixture was added tert-butyl N-{6-oxospiro[3.3]heptan-2-yl}carbamate (500 mg, 2.219 mmol, 1 equiv) in THF (5 mL) dropwise over 3 min at ° C. The resulting mixture was stirred for an additional 1 h at room temperature. The reaction was quenched with sat. NH₄Cl (aq.) at 0° C. and extracted with EtOAc (3×50 mL). The combined organic layers were washed with brine (2×30 mL), dried over anhydrous Na₂SO₄. After filtration, the filtrate was concentrated under reduced pressure. The residue was purified by reversed-phase flash chromatography to afford tert-butyl (6-(2-fluoro-4-(trifluoromethyl)phenyl)-6-hydroxyspiro[3.3]heptan-2-yl)carbamate (150 mg, 17%) as an off-white solid. LC-MS (ES, m/z)=375.10 [M-tBu+ACN+1]⁺.

Step 2. Preparation of tert-butyl (6-(2-fluoro-4-(trifluoromethyl)phenyl)spiro[3.3]hept-5-en-2-yl)carbamate. To a stirred mixture of tert-butyl (6-(2-fluoro-4-(trifluoromethyl)phenyl)-6-hydroxyspiro[3.3]heptan-2-yl)carbamate (140 mg, 0.360 mmol, 1 equiv) and Burgess reagent (214.2 mg, 0.899 mmol, 2.50 equiv) in Toluene (3 mL). The resulting mixture was stirred for 2 h at 80° C. under nitrogen atmosphere. The mixture was allowed to cool down to room temperature. The resulting mixture was diluted with water (50 mL) and extracted with EtOAc (3×30 mL). The combined organic layers were washed with brine (2×20 mL), dried over anhydrous Na₂SO₄. After filtration, the filtrate was concentrated under reduced pressure. The residue was purified by reversed-phase flash chromatography to afford tert-butyl (6-(2-fluoro-4-(trifluoromethyl)phenyl)spiro[3.3]hept-5-en-2-yl)carbamate (80 mg, 60%) as a yellow solid. LC-MS (ES, m/z)=357.10 [M-tBu+ACN+1]+.

Step 3. Preparation of 6-(2-fluoro-4-(trifluoromethyl)phenyl)spiro[3.3]hept-5-en-2-amine. To a stirred solution of tert-butyl (6-(2-fluoro-4-(trifluoromethyl)phenyl)spiro[3.3]hept-5-en-2-yl)carbamate (80 mg, 0.215 mmol, 1 equiv) in DCM (2 mL) was added TFA (0.4 mL) dropwise at 0° C. under nitrogen atmosphere. The resulting mixture was stirred for 1 h at room temperature under nitrogen atmosphere. The resulting mixture was concentrated under vacuum to afford 6-(2-fluoro-4-(trifluoromethyl)phenyl)spiro[3.3]hept-5-en-2-amine (100 mg, crude) as a brown oil. The crude product was used in the next step directly without further purification. LC-MS (ES, m/z)=313.10 [M+ACN+1]⁺.

Step 4. Preparation of 2-(2,6-dioxopiperidin-3-yl)-N-(6-(2-fluoro-4-(trifluoromethyl)phenyl)spiro[3.3]hept-5-en-2-yl)-1-oxoisoindoline-5-carboxamide. To a stirred mixture of 2-(2,6-dioxopiperidin-3-yl)-1-oxo-3H-isoindole-5-carboxylic acid (50 mg, 0.173 mmol, 1 equiv) and 6-[2-fluoro-4-(trifluoromethyl)phenyl]spiro[3.3]hept-5-en-2-amine (94.1 mg, 0.346 mmol, 2 equiv) in DMF (1.5 mL) was added NMI (71.21 mg, 0.865 mmol, 5 equiv) and TCFH (73.0 mg, 0.259 mmol, 1.5 equiv) in portions at room temperature under nitrogen atmosphere. The resulting mixture was stirred for 1 h at room temperature under nitrogen atmosphere. The resulting mixture was diluted with water (20 mL) and extracted with EtOAc (3×10 mL). The combined organic layers were washed with brine (2×20 mL) and concentrated under reduced pressure. The residue was purified by reversed-phase flash chromatography to afford product (50 mg). The crude product was purified by Prep-HPLC to afford 2-(2,6-dioxopiperidin-3-yl)-N-(6-(2-fluoro-4-(trifluoromethyl)phenyl)spiro[3.3]hept-5-en-2-yl)-1-oxoisoindoline-5-carboxamide (15 mg, 16%) as an off-white solid. LC-MS (ES, m/z)=542.20 [M+1]⁺. ¹H NMR (400 MHz, DMSO-d₆) δ 11.01 (s, 1H), 8.87 (d, J=7.3 Hz, 1H), 8.08 (s, 1H), 8.00 (dd, J=7.9, 1.4 Hz, 1H), 7.82 (d, J=7.9 Hz, 1H), 7.69 (dt, J=10.7, 2.1 Hz, 1H), 7.63-7.47 (m, 2H), 6.60 (d, J=3.0 Hz, 1H), 5.15 (dd, J=13.3, 5.1 Hz, 1H), 4.51 (td, J=11.9, 11.2, 6.5 Hz, 2H), 4.40 (d, J=17.5 Hz, 1H), 3.01-2.84 (m, 3H), 2.66-2.53 (m, 2H), 2.48-2.36 (m, 3H), 2.04 (dtd, J=11.0, 5.5, 2.9 Hz, 1H).

### Example 258

### Synthesis of 2-(2,6-dioxopiperidin-3-yl)-N-(6-(2-fluoro-4-(trifluoromethyl)phenyl)spiro[3.3]heptan-2-yl)-1-oxoisoindoline-5-carboxamide

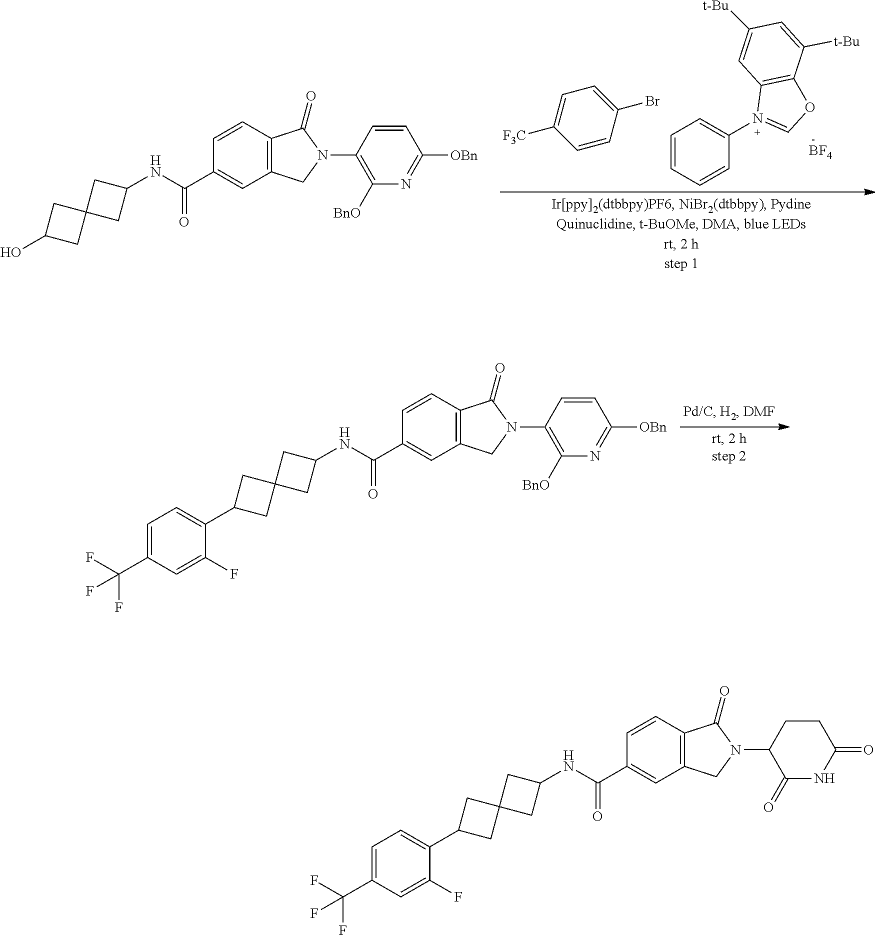

Step 1. Preparation of 2-(2,6-bis(benzyloxy)pyridin-3-yl)-N-(6-(2-fluoro-4-(trifluoromethyl)phenyl)spiro[3.3]heptan-2-yl)-1-oxoisoindoline-5-carboxamide. To a stirred mixture of 2-[2,6-bis(benzyloxy)pyridin-3-yl]-N-{6-hydroxyspiro[3.3]heptan-2-yl}-1-oxo-3H-isoindole-5-carboxamide (150 mg, 0.261 mmol, 1 equiv) and 5,7-di-tert-butyl-3-phenylbenzo[d]oxazol-3-ium tetrafluoroborate (164.8 mg, 0.417 mmol, 1.60 equiv) in 2-methoxy-2-methylpropane (3 mL) was added Pyridine (0.06 mL, 0.783 mmol, 3 equiv) dropwise at 0° C. under nitrogen atmosphere. The resulting mixture was stirred for 10 min at 0° C. under nitrogen atmosphere. Then, the resulting mixture was added to a mixture of 1-bromo-2-fluoro-4-(trifluoromethyl)benzene (95.0 mg, 0.391 mmol, 1.50 equiv), [Ir(dtbbpy)(ppy)2][PF6] (2.4 mg, 0.003 mmol, 0.01 equiv), NiBr₂(dtbbpy) (6.4 mg, 0.013 mmol, 0.05 equiv) and 1-azabicyclo[2.2.2]octane (50.7 mg, 0.456 mmol, 1.75 equiv) in DMA (3 mL) at room temperature under nitrogen atmosphere. The resulting mixture was stirred for 2 h at room temperature under blue LEDs atmosphere. The resulting mixture was diluted with water (50 mL) and extracted with EtOAc (3×20 mL). The combined organic layers were washed with brine (2×30 mL), dried over anhydrous Na₂SO₄. After filtration, the filtrate was concentrated under reduced pressure. The residue was purified by reversed-phase flash chromatography to afford 2-(2,6-bis(benzyloxy)pyridin-3-yl)-N-(6-(2-fluoro-4-(trifluoromethyl)phenyl)spiro[3.3]heptan-2-yl)-1-oxoisoindoline-5-carboxamide (130 mg, 69%) as an off-white solid. LC-MS (ES, m/z)=722.35 [M+1]⁺.

Step 2. Preparation of 2-(2,6-dioxopiperidin-3-yl)-N-(6-(2-fluoro-4-(trifluoromethyl)phenyl)spiro[3.3]heptan-2-yl)-1-oxoisoindoline-5-carboxamide. To a solution of 2-(2,6-bis(benzyloxy)pyridin-3-yl)-N-(6-(2-fluoro-4-(trifluoromethyl)phenyl)spiro[3.3]heptan-2-yl)-1-oxoisoindoline-5-carboxamide (10 mg, 0.014 mmol, 1 equiv) in DMF (2.5 mL) was added Pd/C (60 mg, Palladium 10% on Carbon (wetted with ca. 55% Water)). The mixture was hydrogenated at room temperature for 2 h under hydrogen atmosphere using a hydrogen balloon. The resulting mixture was filtered through a Celite pad, the filter cake was washed with THF (3×5 mL). The filtrate was concentrated under reduced pressure. The residue was purified by reversed-phase flash chromatography to afford 2-(2,6-dioxopiperidin-3-yl)-N-(6-(2-fluoro-4-(trifluoromethyl)phenyl)spiro[3.3]heptan-2-yl)-1-oxoisoindoline-5-carboxamide (56 mg, 62%) as an off-white solid. LC-MS (ES, m/z)=544.20 [M+1]⁺. ¹H NMR (400 MHz, DMSO-d₆) δ 11.01 (s, 1H), 8.79 (d, J=7.3 Hz, 1H), 8.06 (s, 1H), 7.98 (dd, J=8.0, 1.5 Hz, 1H), 7.84-7.77 (m, 1H), 7.61-7.51 (m, 3H), 5.14 (dd, J=13.3, 5.1 Hz, 1H), 4.52 (d, J=17.5 Hz, 1H), 4.38 (dd, J=17.2, 10.4 Hz, 2H), 3.65 (p, J=8.9 Hz, 1H), 2.92 (ddd, J=17.2, 13.6, 5.4 Hz, 1H), 2.65-2.52 (m, 3H), 2.49-2.32 (m, 2H), 2.31-2.16 (m, 4H), 2.10 (dd, J=11.3, 8.8 Hz, 1H), 2.07-1.99 (m, 1H).

### Example 259

### Synthesis of 3-(6-(7-(trifluoromethyl)-2-azaspiro[3.5]nonane-2-carbonyl benzo[d]oxazol-2-yl)piperidine-2,6-dione

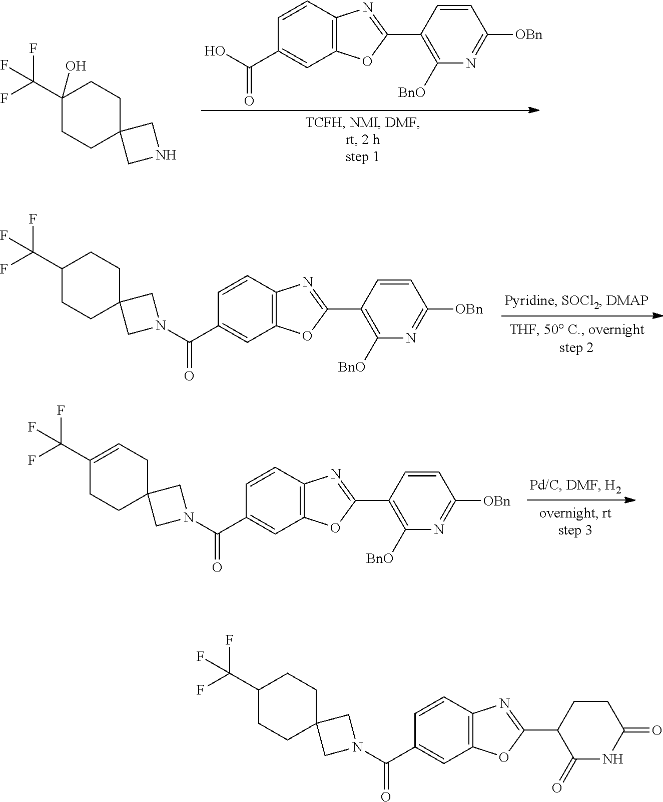 [SMILES: OC1(C(F)(F)F)CCC2(CC1)CNC2 ; O=C(O)c1ccc2nc(-c3ccc(OCc4ccccc4)nc3OCc3ccccc3)oc2c1 ; O=C(c1ccc2nc(-c3ccc(OCc4ccccc4)nc3OCc3ccccc3)oc2c1)N1CC2(CCC(C(F)(F)F)CC2)C1 ; O=C(c1ccc2nc(-c3ccc(OCc4ccccc4)nc3OCc3ccccc3)oc2c1)N1CC2(CC=C(C(F)(F)F)CC2)C1 ; O=C1CCC(c2nc3ccc(C(=O)N4CC5(CCC(C(F)(F)F)CC5)C4)cc3o2)C(=O)N1]

Step 1. Preparation of (2-(2,6-bis(benzyloxy)pyridin-3-yl)benzo[d]oxazol-6-yl)(7-hydroxy-7-(trifluoromethyl)-2-azaspiro[3.5]nonan-2-yl)methanone. To a stirred mixture of 2-[2,6-bis(benzyloxy)pyridin-3-yl]-1,3-benzoxazole-6-carboxylic acid (800 mg, 1.768 mmol, 1.00 equiv) and 7-(trifluoromethyl)-2-azaspiro[3.5]nonan-7-ol (554.9 mg, 2.652 mmol, 1.50 equiv) in DMF (15 mL) was added NMI (725.9 mg, 8.841 mmol, 5.00 equiv) and TCFH (744.2 mg, 2.652 mmol, 1.50 equiv) in portions at room temperature under nitrogen atmosphere. The resulting mixture was stirred for 2 h at room temperature under nitrogen atmosphere. The resulting mixture was diluted with water (100 mL) and extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography to afford (2-(2,6-bis(benzyloxy)pyridin-3-yl)benzo[d]oxazol-6-yl)(7-hydroxy-7-(trifluoromethyl)-2-azaspiro[3.5]nonan-2-yl)methanone (880 mg, 77%) as an off-white solid. LC-MS (ES, m/z)=644.25 [M+1]⁺.

Step 2. Preparation of (2-(2,6-bis(benzyloxy)pyridin-3-yl)benzo[d]oxazol-6-yl)(7-(trifluoromethyl)-2-azaspiro[3.5]non-6-en-2-yl)methanone. A mixture of (2-(2,6-bis(benzyloxy)pyridin-3-yl)benzo[d]oxazol-6-yl)(7-hydroxy-7-(trifluoromethyl)-2-azaspiro[3.5]nonan-2-yl)methanone (220 mg, 0.342 mmol, 1 equiv), Pyridine (81.1 mg, 1.026 mmol, 3 equiv), SOCl₂ (121.9 mg, 1.026 mmol, 3 equiv) and DMAP (4.1 mg, 0.034 mmol, 0.1 equiv) in THF (3 mL) was stirred for overnight at 50° C. under nitrogen atmosphere. The mixture was allowed to cool down to room temperature. The resulting mixture was filtered, the filter cake was washed with THF (3×5 mL). The filtrate was concentrated under reduced pressure. The residue was purified by reversed-phase flash chromatography to afford (2-(2,6-bis(benzyloxy)pyridin-3-yl)benzo[d]oxazol-6-yl)(7-(trifluoromethyl)-2-azaspiro[3.5]non-6-en-2-yl)methanone (130 mg, 61%) as a yellow solid. LC-MS (ES, m/z)=626.25 [M+1]⁺.

Step 3. Preparation of 3-(6-(7-(trifluoromethyl)-2-azaspiro[3.5]nonane-2-carbonyl)benzo[d]oxazol-2-yl)piperidine-2,6-dione. A mixture of (2-(2,6-bis(benzyloxy)pyridin-3-yl)benzo[d]oxazol-6-yl)(7-(trifluoromethyl)-2-azaspiro[3.5]non-6-en-2-yl)methanone (120 mg, 0.192 mmol, 1 equiv) in DMF (2 mL) was added Pd/C (120 mg, Palladium 10% on Carbon (wetted with ca. 55% Water)) at room temperature. The resulting mixture was stirred overnight at room temperature under hydrogen atmosphere. The resulting mixture was filtered through a Celite pad, the filter cake was washed with THF (3×5 mL). The filtrate was concentrated under reduced pressure. The residue was purified by Prep-HPLC to afford 3-(6-(7-(trifluoromethyl)-2-azaspiro[3.5]nonane-2-carbonyl)benzo[d]oxazol-2-yl)piperidine-2,6-dione (28 mg, 32%) as an off-white solid. LC-MS (ES, m/z)=450.14 [M+1]⁺. ¹H NMR (400 MHz, DMSO-d₆) δ 11.15 (s, 1H), 8.02 (d, J=3.5 Hz, 1H), 7.81 (d, J=8.3 Hz, 1H), 7.68 (t, J=8.0 Hz, 1H), 6.37 (s, 1H), 4.56 (dd, J=11.3, 5.0 Hz, 1H), 4.24-3.96 (m, 2H), 3.95-3.69 (m, 2H), 2.87-2.57 (m, 2H), 2.49-2.27 (m, 3H), 2.22 (s, 1H), 1.98 (d, J=13.1 Hz, 1H), 1.87 (q, J=6.0 Hz, 1H), 1.78 (s, 1H), 1.57-1.43 (m, 1H), 1.26 (d, J=15.8 Hz, 1H).

### Example 260

### Synthesis of (2-(2,6-dioxopiperidin-3-yl)benzo[d]oxazol-5-yl)methyl (7,7-difluorospiro[3.5]nonan-2-yl)carbamate

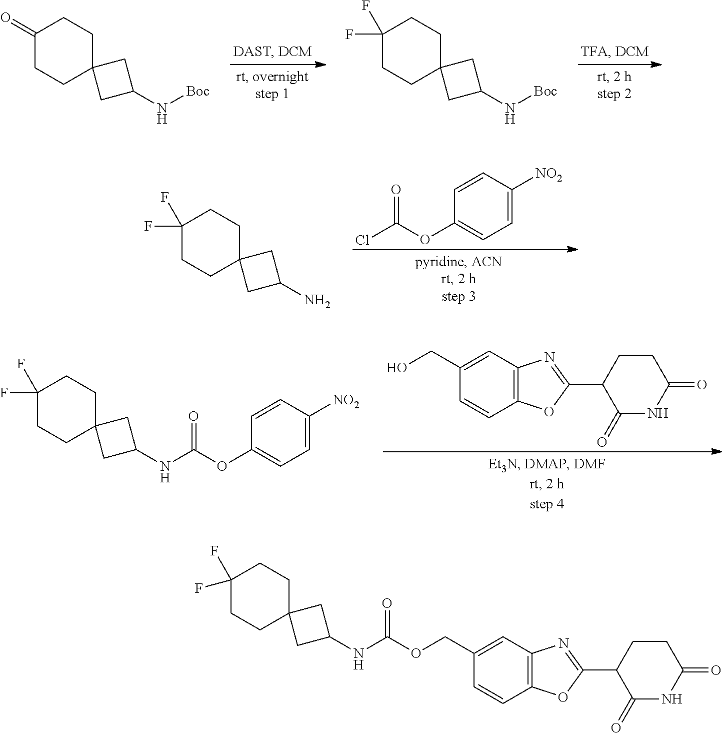

Step 1. Preparation of tert-butyl (7,7-difluorospiro[3.5]nonan-2-yl)carbamate. To a stirred solution of tert-butyl N-{7-oxospiro[3.5]nonan-2-yl}carbamate (500 mg, 1.974 mmol, 1 equiv) in DCM (10 mL) was added BAST (1.00 mL, 5.428 mmol, 2.75 equiv) dropwise at 0° C. under nitrogen atmosphere. The resulting mixture was stirred overnight at room temperature under nitrogen atmosphere. The reaction was quenched with sat. NH₄Cl (aq.) at 0° C. The resulting mixture was diluted with water (50 mL) and extracted with DCM (3×30 mL). The combined organic layers were washed with brine (2×30 mL), dried over anhydrous Na₂SO₄. After filtration, the filtrate was concentrated under reduced pressure. The residue was purified by reversed-phase flash chromatography to afford tert-butyl (7,7-difluorospiro[3.5]nonan-2-yl)carbamate (500 mg, 92%) as an off-white solid. LC-MS (ES, m/z)=261.05 [M-tBu+ACN+1]⁺.

Step 2. Preparation of 7,7-difluorospiro[3.5]nonan-2-amine. To a stirred solution of tert-butyl (7,7-difluorospiro[3.5]nonan-2-yl)carbamate (500 mg, 1.816 mmol, 1 equiv) in DCM (10 mL) was added TFA (2 mL) dropwise at 0° C. under nitrogen atmosphere. The resulting mixture was stirred for 2 h at room temperature under nitrogen atmosphere. The resulting mixture was concentrated under reduced pressure to afford 7,7-difluorospiro[3.5]nonan-2-amine (500 mg, crude) as an off-white solid. The residue was used directly in the next step without further purification. LC-MS (ES, m/z)=217.10 [M+ACN+1]⁺.

Step 3. Preparation of 4-nitrophenyl (7,7-difluorospiro[3.5]nonan-2-yl)carbamate. To a stirred mixture of 7,7-difluorospiro[3.5]nonan-2-amine (300 mg, 1.712 mmol, 1 equiv) and pyridine (677.2 mg, 8.561 mmol, 5.00 equiv) in ACN (5 mL) was added 4-nitrophenyl carbonochloridate (517.7 mg, 2.568 mmol, 1.50 equiv) in ACN (1 mL) dropwise at 0° C. under nitrogen atmosphere. The resulting mixture was stirred for 2 h at room temperature under nitrogen atmosphere. The resulting mixture was diluted with water (50 mL) and extracted with EtOAc (3×30 mL). The combined organic layers were washed with brine (2×50 mL), dried over anhydrous Na₂SO₄. After filtration, the filtrate was concentrated under reduced pressure. The residue was purified by reversed-phase flash chromatography to afford 4-nitrophenyl (7,7-difluorospiro[3.5]nonan-2-yl)carbamate (200 mg, 34%) as an off-white solid. ¹H NMR (400 MHz, DMSO-d₆) δ 8.40-8.31 (m, 1H), 8.30-8.21 (m, 2H), 8.16-8.08 (m, 1H), 7.44-7.36 (m, 2H), 6.97-6.89 (m, 1H), 4.04 (hept, J=8.0 Hz, 1H), 2.33-2.17 (m, 2H), 1.93-1.73 (m, 6H), 1.62 (dt, J=20.7, 6.4 Hz, 4H).

Step 4. Preparation of (2-(2,6-dioxopiperidin-3-yl)benzo[d]oxazol-5-yl)methyl (7,7-difluorospiro[3.5]nonan-2-yl)carbamate. To a stirred mixture of 3-[5-(hydroxymethyl)-1,3-benzoxazol-2-yl]piperidine-2,6-dione (40 mg, 0.154 mmol, 1.00 equiv), 4-nitrophenyl (7,7-difluorospiro[3.5]nonan-2-yl)carbamate (78.5 mg, 0.231 mmol, 1.50 equiv) and DMAP (18.8 mg, 0.154 mmol, 1.00 equiv) in DMF (1 mL) was added Et₃N (0.06 mL, 0.462 mmol, 3 equiv) at room temperature under nitrogen atmosphere. The resulting mixture was stirred for 2 h at room temperature under nitrogen atmosphere. The mixture was purified by reversed-phase flash chromatography to afford (2-(2,6-dioxopiperidin-3-yl)benzo[d]oxazol-5-yl)methyl (7,7-difluorospiro[3.5]nonan-2-yl)carbamate (8.7 mg, 12%) as an off-white solid. LC-MS (ES, m/z)=462.15 [M+1]⁺. ¹H NMR (400 MHz, DMSO-d₆) δ 11.13 (s, 1H), 7.77-7.69 (m, 2H), 7.56 (d, J=7.8 Hz, 1H), 7.40 (dd, J=8.5, 1.6 Hz, 1H), 5.11 (s, 2H), 4.51 (dd, J=11.3, 5.0 Hz, 1H), 3.97 (h, J=8.2 Hz, 1H), 3.17 (q, J=7.0 Hz, 1H), 2.79-2.56 (m, 2H), 2.42 (dtd, J=13.2, 11.3, 4.8 Hz, 1H), 2.30 (dq, J=13.0, 4.8 Hz, 1H), 2.18-2.02 (m, 3H), 1.88-1.73 (m, 4H), 1.72-1.64 (m, 3H), 1.63-1.58 (m, 2H), 1.55 (q, J=6.2, 5.6 Hz, 2H), 0.99 (t, J=7.0 Hz, 1H).

### Example 261

### Synthesis of 3-(5-(4-chloro-7-(trifluoromethyl)isoindoline-2-carbonyl)-1-oxoisoindolin-2-yl)piperidine-2,6-dione

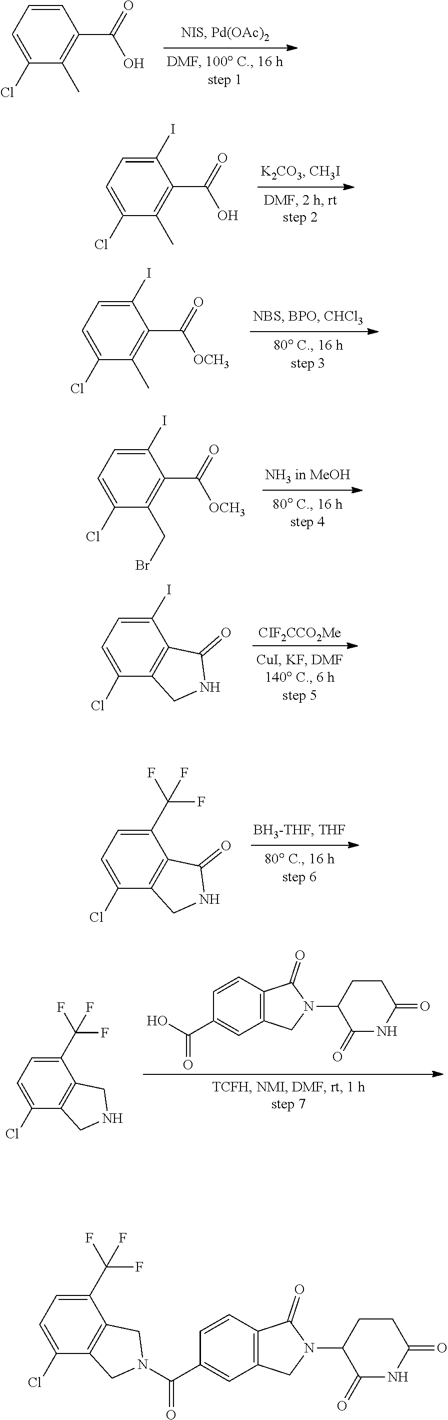

Step 1. Preparation of 3-chloro-6-iodo-2-methylbenzoic acid. A solution of 3-chloro-2-methylbenzoic acid (5 g, 29.310 mmol, 1 equiv), NIS (7.25 g, 32.241 mmol, 1.1 equiv) and Pd(OAc)₂ (0.66 g, 2.931 mmol, 0.1 equiv) in dimethylformamide (50 mL) was stirred overnight at 100° C. under nitrogen atmosphere. The resulting mixture was diluted with water (500 mL) and extracted with EtOAc (3×200 mL). The combined organic layers were washed with Na₂S₂O₃ (aq) (2×500 mL) and brine (2×50 mL), dried over anhydrous Na₂SO₄. After filtration, the filtrate was concentrated under reduced pressure to afford 3-chloro-6-iodo-2-methylbenzoic acid (8.6 g, crude) as a yellow solid. LC-MS (ES, m/z)=294.85 [M−1]⁻.

Step 2. Preparation of methyl 3-chloro-6-iodo-2-methylbenzoate. To a stirred mixture of 3-chloro-6-iodo-2-methylbenzoic acid (8.5 g, 28.669 mmol, 1 equiv) and K₂CO₃ (11.89 g, 86.007 mmol, 3 equiv) in DMF (50 mL) were added CH₃I (2.68 mL, 43.004 mmol, 1.5 equiv) dropwise at 0° C. under nitrogen atmosphere. The resulting mixture was stirred for 2 h at room temperature under nitrogen atmosphere. The reaction was quenched with water at 0° C. and extracted with EtOAc (3×200 mL). The combined organic layers were washed with water (2×500 mL), dried over anhydrous Na₂SO₄. After filtration, the filtrate was concentrated under reduced pressure. The residue was purified by silica gel column chromatography, eluted with PE/EA (9:1) to afford methyl 3-chloro-6-iodo-2-methylbenzoate (5.5 g, 62%) as a yellow solid. ¹H NMR (400 MHz, DMSO-d₆) δ 7.74 (d, J=8.4 Hz, 1H), 7.31 (d, J=8.4 Hz, 1H), 3.90 (s, 3H), 2.27 (s, 3H).

Step 3. Preparation of 2-(bromomethyl)-3-chloro-6-iodobenzoate. To a stirred mixture of methyl 3-chloro-6-iodo-2-methylbenzoate (2 g, 6.441 mmol, 1 equiv) and BPO (0.17 g, 0.644 mmol, 0.1 equiv) in CHCl₃ (20 mL) was added NBS (1.72 g, 9.662 mmol, 1.5 equiv) at room temperature under air atmosphere. The resulting mixture was stirred overnight at 80° C. under nitrogen atmosphere. The mixture was allowed to cool down to room temperature. The resulting mixture was diluted with water (200 mL) and extracted with DCM (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford 2-(bromomethyl)-3-chloro-6-iodobenzoate (2.1 g, 84%) as a colorless oil. ¹H NMR (400 MHz, DMSO-d₆) δ 7.84 (dd, J=8.0, 2.5 Hz, 1H), 7.53 (dd, J=9.3, 2.5 Hz, 1H), 4.61 (s, 2H), 3.90 (s, 3H).

Step 4. Preparation of 4-chloro-7-iodoisoindolin-1-one. A mixture of methyl 2-(bromomethyl)-3-chloro-6-iodobenzoate (2.1 g, 5.415 mmol, 1 equiv) and NH₃(7M) in MeOH (20 mL) was stirred overnight at 80° C. under nitrogen atmosphere. The mixture was allowed to cool down to room temperature. The mixture was concentrated under reduced pressure. The residue was purified by silica gel column chromatography, eluted with PE/EA (1:2) to afford 4-chloro-7-iodoisoindolin-1-one (1.0 g, 63%) as an off-white solid. LC-MS (ES, m/z)=293.85 [M+1]⁺.

Step 5. Preparation of 4-chloro-7-(trifluoromethyl)isoindolin-1-one. To a stirred mixture of 4-chloro-7-iodoisoindolin-1-one (1 g, 3.407 mmol, 1 equiv) and methyl 2-chloro-2,2-difluoroacetate (984.7 mg, 6.815 mmol, 2.00 equiv) in DMF (10 mL) was added CuI (778.7 mg, 4.089 mmol, 1.20 equiv) and KF (237.6 mg, 4.090 mmol, 1.20 equiv) at room temperature under nitrogen atmosphere. The resulting mixture was stirred for 6 h at 140° C. under nitrogen atmosphere. The mixture was allowed to cool down to room temperature. The resulting mixture was diluted with water (100 mL) and extracted with EtOAc (3×50 mL). The combined organic layers were concentrated under reduced pressure. The residue was purified by reversed-phase flash chromatography to afford 4-chloro-7-(trifluoromethyl)isoindolin-1-one (200 mg, 25%) as an off-white solid. LC-MS (ES, m/z)=233.75 [M−1]⁻.

Step 6. Preparation of 4-chloro-7-(trifluoromethyl)isoindoline. To a stirred solution of 4-chloro-7-(trifluoromethyl)isoindolin-1-one (175 mg, 0.743 mmol, 1 equiv) in THF (2 mL) was added tetrahydrofuran borane (3.71 mL, 3.715 mmol, 5 equiv) under nitrogen atmosphere. The resulting mixture was stirred overnight at 80° C. under nitrogen atmosphere. The reaction was quenched with 1N HCl at 0° C. The resulting mixture was concentrated under reduced pressure. The residue was purified by reversed-phase flash chromatography to afford 4-chloro-7-(trifluoromethyl)isoindoline (50 mg, 30%) as an off-white solid. LC-MS (ES, m/z)=222.0 [M+1]⁺.

Step 7. Preparation of 3-(5-(4-chloro-7-(trifluoromethyl)isoindoline-2-carbonyl)-1-oxoisoindolin-2-yl)piperidine-2,6-dione. To a stirred mixture of 4-chloro-7-(trifluoromethyl)isoindoline (46.13 mg, 0.208 mmol, 1 equiv) and 2-(2,6-dioxopiperidin-3-yl)-1-oxo-3H-isoindole-5-carboxylic acid (60 mg, 0.208 mmol, 1.00 equiv) in DMF (1.5 mL) were added NMI (85.45 mg, 1.040 mmol, 5 equiv) and TCFH (87.60 mg, 0.312 mmol, 1.5 equiv) in portions at room temperature under nitrogen atmosphere. The resulting mixture was stirred for 1 h at room temperature under nitrogen atmosphere. The resulting mixture was diluted with water (30 mL) and extracted with DCM (3×20 mL). The combined organic layers were washed with brine (2×50 mL), dried over anhydrous Na₂SO₄. After filtration, the filtrate was concentrated under reduced pressure. The crude product was purified by Prep-HPLC to afford 3-(5-(4-chloro-7-(trifluoromethyl)isoindoline-2-carbonyl)-1-oxoisoindolin-2-yl)piperidine-2,6-dione (10 mg, 9.77%) as an off-white solid. LC-MS (ES, m/z)=494.95 [M+1]⁺. ¹H NMR (400 MHz, DMSO-d₆) δ 11.01 (s, 1H), 7.85 (dd, J=7.9, 2.3 Hz, 2H), 7.79-7.71 (m, 2H), 7.66 (dd, J=15.0, 8.3 Hz, 1H), 5.16 (dd, J=13.4, 5.0 Hz, 1H), 5.10 (s, 1H), 4.98 (d, J=20.5 Hz, 2H), 4.85 (s, 1H), 4.56 (d, J=18.0 Hz, 1H), 4.47-4.38 (m, 1H), 2.93 (ddd, J=17.8, 13.5, 5.3 Hz, 1H), 2.62 (d, J=17.1 Hz, 1H), 2.41 (td, J=13.2, 4.6 Hz, 1H), 2.11-2.00 (m, 1H).

### Example 262

### Synthesis of (2-(2,6-dioxopiperidin-3-yl)benzo[d]oxazol-5-yl)methyl 6,6-difluoro-2-azaspiro[3.3]heptane-2-carboxylate

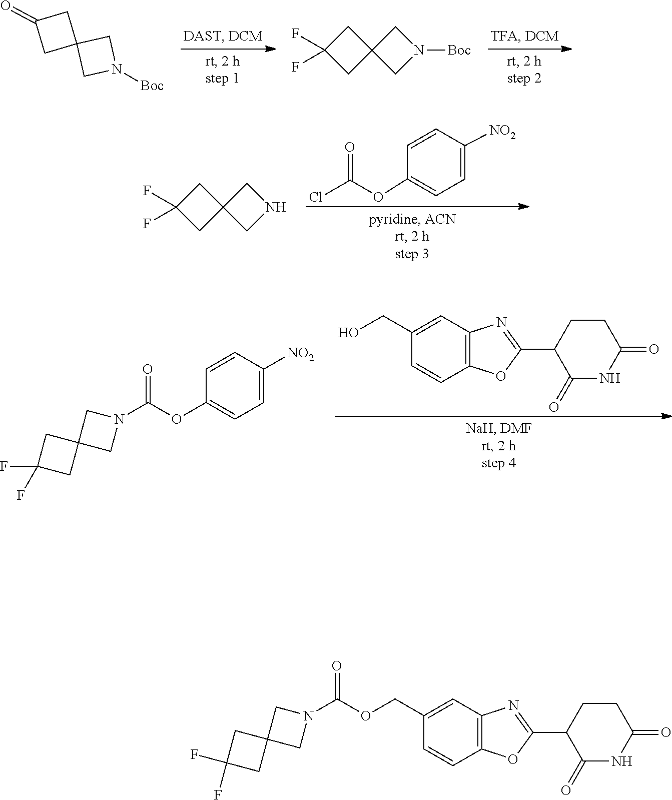

Step 1. Preparation of tert-butyl 6,6-difluoro-2-azaspiro[3.3]heptane-2-carboxylate. To a stirred mixture of tert-butyl 6-oxo-2-azaspiro[3.3]heptane-2-carboxylate (600 mg, 2.840 mmol, 1 equiv) in DCM (10 mL) was added DAST (1.83 g, 11.360 mmol, 4 equiv) dropwise at 0° C. under nitrogen atmosphere. The resulting mixture was stirred for 2 h at room temperature under nitrogen atmosphere. The reaction was quenched with sat. NH₄Cl (aq.) at 0° C. The resulting mixture was diluted with water (100 mL) and extracted with DCM (3×50 mL). The combined organic layers were washed with brine (2×100 mL), dried over anhydrous Na₂SO₄. After filtration, the filtrate was concentrated under reduced pressure. The residue was purified by reversed-phase flash chromatography to afford tert-butyl 6,6-difluoro-2-azaspiro[3.3]heptane-2-carboxylate (360 mg, 54%) as an off-white solid. LC-MS (ES, m/z)=219.05 [M-tBu+ACN+1]⁺.

Step 2. Preparation of 6,6-difluoro-2-azaspiro[3.3]heptane. A mixture of tert-butyl 6,6-difluoro-2-azaspiro[3.3]heptane-2-carboxylate (350 mg, 1.500 mmol, 1 equiv) in DCM (5 mL) was added TFA (1 mL) dropwise at 0° C. under nitrogen atmosphere. The resulting mixture was stirred for 2 h at room temperature under nitrogen atmosphere. The resulting mixture was concentrated under vacuum to afford 6,6-difluoro-2-azaspiro[3.3]heptane (400 mg, crude) as a colorless oil. The residue was used for the next step without further purification. LC-MS (ES, m/z)=134.35 [M+1]⁺.

Step 3. Preparation of 4-nitrophenyl 6,6-difluoro-2-azaspiro[3.3]heptane-2-carboxylate. To a stirred mixture of 6,6-difluoro-2-azaspiro[3.3]heptane (400 mg, 3.004 mmol, 1 equiv) and pyridine (712.92 mg, 9.012 mmol, 3 equiv) in ACN (4 mL) was added 4-nitrophenyl carbonochloridate (1.21 g, 6.008 mmol, 2 equiv) in ACN (2 mL) dropwise at 0° C. under nitrogen atmosphere. The resulting mixture was stirred for 2 h at room temperature under nitrogen atmosphere. The reaction was quenched with water at 0° C. The resulting mixture was diluted with water (50 mL) and extracted with DCM (3×30 mL). The combined organic layers were washed with brine (2×30 mL), dried over anhydrous Na₂SO₄. After filtration, the filtrate was concentrated under reduced pressure. The residue was purified by reversed-phase flash chromatography to afford 4-nitrophenyl 6,6-difluoro-2-azaspiro[3.3]heptane-2-carboxylate (280 mg, 31%) as a brown solid. ¹H NMR (400 MHz, DMSO-d₆) δ 8.31-8.23 (m, 2H), 7.47-7.38 (m, 2H), 4.31 (s, 4H), 2.88 (t, J=12.5 Hz, 4H).

Step 4. Preparation of (2-(2,6-dioxopiperidin-3-yl)benzo[d]oxazol-5-yl)methyl 6,6-difluoro-2-azaspiro[3.3]heptane-2-carboxylate. To a stirred solution of 3-[5-(hydroxymethyl)-1,3-benzoxazol-2-yl]piperidine-2,6-dione (50 mg, 0.192 mmol, 1.00 equiv) in DMF (1 mL) was added NaH (19.2 mg, 0.480 mmol, 2.50 equiv, 60%) in portions at 0° C. under nitrogen atmosphere. The resulting mixture was stirred for 30 min at 0° C. under nitrogen atmosphere. To the above mixture was added 4-nitrophenyl 6,6-difluoro-2-azaspiro[3.3]heptane-2-carboxylate (68.8 mg, 0.231 mmol, 1.20 equiv) in DMF (1 mL) dropwise at 0° C. The resulting mixture was stirred for an additional 30 min at 0° C. The reaction was quenched by the addition of sat. NH₄Cl (aq.) at 0° C. The resulting mixture was purified by reversed-phase flash chromatography to afford product (30 mg). The crude product was purified by Prep-HPLC to afford (2-(2,6-dioxopiperidin-3-yl)benzo[d]oxazol-5-yl)methyl 6,6-difluoro-2-azaspiro[3.3]heptane-2-carboxylate (12 mg, 14%) as an off-white solid. LC-MS (ES, m/z)=419.95 [M+1]⁺. ¹H NMR (400 MHz, DMSO-d₆) δ 11.13 (s, 1H), 7.76-7.69 (m, 2H), 7.41 (dd, J=8.5, 1.6 Hz, 1H), 5.14 (s, 2H), 4.51 (dd, J=11.2, 5.1 Hz, 1H), 2.81 (t, J=12.5 Hz, 4H), 2.76-2.58 (m, 2H), 2.42 (dtd, J=13.2, 11.2, 4.8 Hz, 1H), 2.31 (dq, J=13.2, 5.1 Hz, 1H).

### Example 263

### Synthesis of 3-(5-(4-fluoro-5-(trifluoromethoxy)isoindoline-2-carbonyl)-1-oxoisoindolin-2-yl)piperidine-2,6-dione

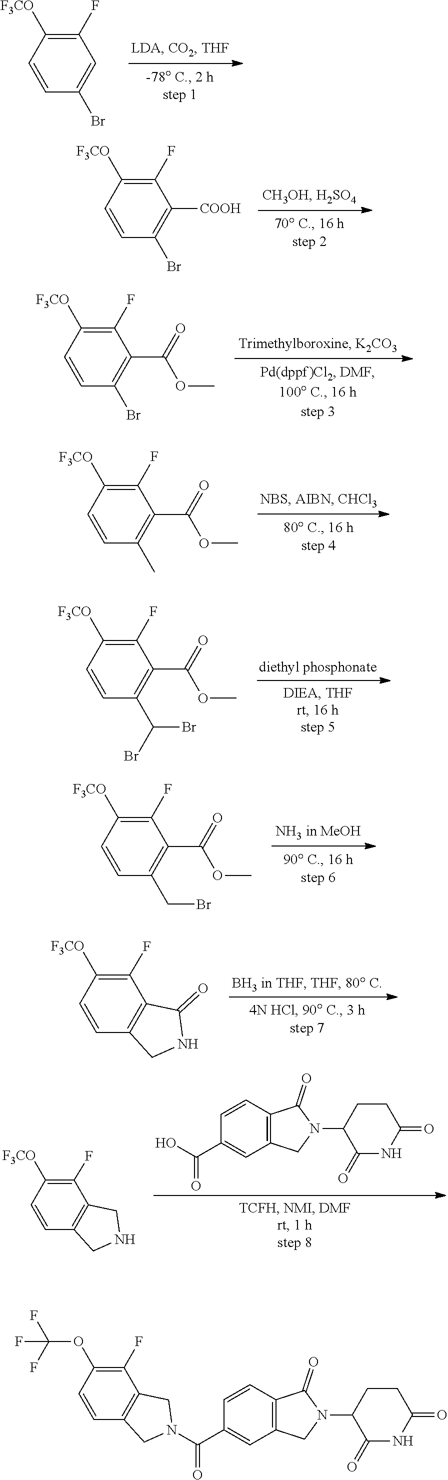

Step 1. Preparation of 6-bromo-2-fluoro-3-(trifluoromethoxy)benzoic acid. To a stirred solution of 4-bromo-2-fluoro-1-(trifluoromethoxy)benzene (7 g, 27.027 mmol, 1 equiv) in THF (140 mL) was added LDA (1M in THF) (16.22 mL, 32.432 mmol, 1.2 equiv) dropwise at −78° C. under nitrogen atmosphere. After stirring for 1 h at −78° C., passed the CO₂ into the resulting mixture slowly at the same temperature. The resulting mixture was stirred for 1 h at −78° C. After warming to room temperature, the solvent was removed under reduced pressure. The residue was diluted with water and extracted with EtOAc (3×200 mL). The aqueous phase was adjusted to pH 3 by HCl (1 M) and extracted with ethyl acetate three times. The combined organic extracts were washed with brine, dried over sodium sulfate, filtered and concentrated under reduced pressure to give 6-bromo-2-fluoro-3-(trifluoromethoxy)benzoic acid (7 g, 86%) as yellow oil. LC-MS (ES, m/z)=301.0 [M−1]⁻.

Step 2. Preparation of methyl 6-bromo-2-fluoro-3-(trifluoromethoxy)benzoate. To a stirred solution of 6-bromo-2-fluoro-3-(trifluoromethoxy)benzoic acid (6.8 g, 22.442 mmol, 1 equiv) in CH₃OH (120 mL) was added H₂SO₄ (12 mL) dropwise at 0° C. The resulting mixture was stirred overnight at 70° C. The reaction was quenched by the addition of Water/Ice (500 mL) at 0° C. The mixture residue was neutralized to pH 5 with saturated NaOH (aq.). The resulting mixture was extracted with EtOAc (3×200 mL). The combined organic layers were washed with brine (2×500 mL), dried over anhydrous Na₂SO₄. After filtration, the filtrate was concentrated under reduced pressure. The residue was purified by silica gel column chromatography, eluted with PE/EA (6:1) to afford methyl 6-bromo-2-fluoro-3-(trifluoromethoxy)benzoate (3.8 g, 53%) as a yellow oil. ¹H NMR (400 MHz, MeOH-d₄) δ 7.61 (dd, J=8.9, 1.8 Hz, 1H), 7.50 (ddq, J=9.1, 7.9, 1.2 Hz, 1H), 3.98 (s, 3H).

Step 3. Preparation of methyl 2-fluoro-6-methyl-3-(trifluoromethoxy)benzoate. To a stirred mixture of [6-bromo-2-fluoro-3-(trifluoromethoxy)cyclohexyl](methoxy)methanol (5.3 g, 16.303 mmol, 1 equiv) and trimethyl-1,3,5,2,4,6-trioxatriborinane (8.19 mL, 32.606 mmol, 2 equiv) in DMF (100 mL) were added K₂CO₃ (6759.38 mg, 48.909 mmol, 3 equiv) and Pd(dppf)Cl₂ (1.19 g, 1.630 mmol, 0.1 equiv) at room temperature under nitrogen atmosphere. The resulting mixture was stirred overnight at 100° C. under nitrogen atmosphere. The resulting mixture was diluted with water (300 mL) and extracted with EtOAc (3×200 mL). The combined organic layers were washed with brine (2×500 mL), dried over anhydrous Na₂SO₄. After filtration, the filtrate was concentrated under reduced pressure. The residue was purified by reversed-phase flash chromatography to afford methyl 2-fluoro-6-methyl-3-(trifluoromethoxy)benzoate (3.2 g, 78%) as a brown oil. ¹H NMR (400 MHz, Chloroform-d) δ 7.05-7.02 (m, 1H), 7.02-6.99 (m, 1H), 3.96 (s, 3H), 2.40 (s, 3H).

Step 4. Preparation of methyl 6-(dibromomethyl)-2-fluoro-3-(trifluoromethoxy)benzoate. To a stirred mixture of methyl 2-fluoro-6-methyl-3-(trifluoromethoxy)benzoate (3 g, 11.886 mmol, 1 equiv) and NBS (3.18 g, 17.87 mmol, 1.5 equiv) in CHCl₃ (45 mL) were added AIBN (390.9 mg, 0.238 mmol, 0.2 equiv) at room temperature under nitrogen atmosphere. The resulting mixture was stirred overnight at 80° C. The resulting mixture was diluted with water (200 mL). The resulting mixture was extracted with DCM (3×100 mL). The combined organic layers were washed with brine (2×100 mL), dried over anhydrous Na₂SO₄. After filtration, the filtrate was concentrated under reduced pressure. The residue was purified by reversed-phase flash chromatography to afford methyl 6-(dibromomethyl)-2-fluoro-3-(trifluoromethoxy)benzoate (1.4 g, 29%) as an white oil. ¹H NMR (400 MHz, Chloroform-d) δ 7.91 (dt, J=8.9, 1.9 Hz, 1H), 7.50 (ddd, J=8.9, 7.5, 1.5 Hz, 1H), 7.13 (d, J=2.0 Hz, 1H), 4.03 (d, J=2.0 Hz, 3H).

Step 5. Preparation of methyl 6-(bromomethyl)-2-fluoro-3-(trifluoromethoxy)benzoate. To a stirred solution of methyl 6-(dibromomethyl)-2-fluoro-3-(trifluoromethoxy)benzoate (1.4 g, 3.415 mmol, 1 equiv) and DIEA (1.77 g, 13.660 mmol, 4 equiv) in THF (28 mL) were added diethyl phosphonate (1.89 g, 13.660 mmol, 4 equiv) at room temperature. The resulting mixture was stirred overnight at room temperature. The resulting mixture was diluted with water (150 mL). The resulting mixture was extracted with DCM (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (5:1) to afford methyl 6-(bromomethyl)-2-fluoro-3-(trifluoromethoxy)benzoate (1.0 g, 88%) as a colorless oil. LC-MS (ES, m/z)=no mass signal.

Step 6. Preparation of 7-fluoro-6-(trifluoromethoxy)isoindolin-1-one. A solution of methyl 6-(bromomethyl)-2-fluoro-3-(trifluoromethoxy)benzoate (1 g, 3.021 mmol, 1 equiv) and NH₃ (7M) in MeOH (10 mL) was stirred overnight at 90° C. The resulting mixture was concentrated under reduced pressure to afford 7-fluoro-6-(trifluoromethoxy)isoindolin-1-one (700 mg, 99%) as a yellow solid. LC-MS (ES, m/z)=277.10 [M+ACN+1]⁺.

Step 7. Preparation of 4-fluoro-5-(trifluoromethoxy)isoindoline. To a stirred solution of 7-fluoro-6-(trifluoromethoxy)isoindolin-1-one (150 mg, 0.638 mmol, 1 equiv) in THF (3 mL) was added BH₃-THF (3.19 mL, 3.190 mmol, 5 equiv) under nitrogen atmosphere. The resulting mixture was stirred overnight at 80° C. under nitrogen atmosphere. The mixture was allowed to cool down to room temperature and quenched with 4N HCl at 0° C. The resulting mixture was stirred at 90° C. for an additional 3 h under nitrogen atmosphere. The resulting mixture was concentrated under reduced pressure. The residue was purified by reversed-phase flash chromatography to afford 4-fluoro-5-(trifluoromethoxy)isoindoline (150 mg, crude) as a brown solid. LC-MS (ES, m/z)=222.0 [M+1]⁺.

Step 8. Preparation of 3-(5-(4-fluoro-5-(trifluoromethoxy)isoindoline-2-carbonyl)-1-oxoisoindolin-2-yl)piperidine-2,6-dione. To a stirred mixture of 4-fluoro-5-(trifluoromethoxy)isoindoline (46.0 mg, 0.208 mmol, 1.20 equiv) and 2-(2,6-dioxopiperidin-3-yl)-1-oxo-3H-isoindole-5-carboxylic acid (50 mg, 0.173 mmol, 1.00 equiv) in DMF (1.0 mL) were added NMI (71.2 mg, 0.867 mmol, 5.00 equiv) and TCFH (73.0 mg, 0.260 mmol, 1.50 equiv) in portions at room temperature under nitrogen atmosphere. The resulting mixture was stirred for 1 h at room temperature under nitrogen atmosphere. The resulting mixture was diluted with water (30 mL) and extracted with EtOAc (3×20 mL). The combined organic layers were washed with brine (2×50 mL) and concentrated under reduced pressure. The mixture was purified by reversed-phase flash chromatography to afford 3-(5-(4-fluoro-5-(trifluoromethoxy)isoindoline-2-carbonyl)-1-oxoisoindolin-2-yl)piperidine-2,6-dione (32 mg, 37%) as an off-white solid. LC-MS (ES, m/z)=492.15 [M+1]⁺. ¹H NMR (400 MHz, DMSO-d₆) δ 11.03 (s, 1H), 7.88-7.80 (m, 2H), 7.73 (dt, J=7.8, 1.8 Hz, 1H), 7.53 (dt, J=14.7, 7.7 Hz, 1H), 7.30 (dd, J=65.4, 8.3 Hz, 1H), 5.16 (dd, J=13.3, 5.1 Hz, 1H), 5.01 (s, 1H), 4.95 (s, 1H), 4.92 (s, 1H), 4.83 (s, 1H), 4.54 (dd, J=17.6, 4.7 Hz, 1H), 4.41 (dd, J=17.7, 4.2 Hz, 1H), 2.93 (ddd, J=17.2, 13.6, 5.4 Hz, 1H), 2.67-2.57 (m, 1H), 2.42 (tt, J=13.2, 6.6 Hz, 1H), 2.04 (ddd, J=9.5, 5.4, 2.6 Hz, 1H).

### Example 264

### Synthesis of 3-(5-(6-fluoro-4-(trifluoromethyl)isoindoline-2-carbonyl)-1-oxoisoindolin-2-yl)piperidine-2,6-dione

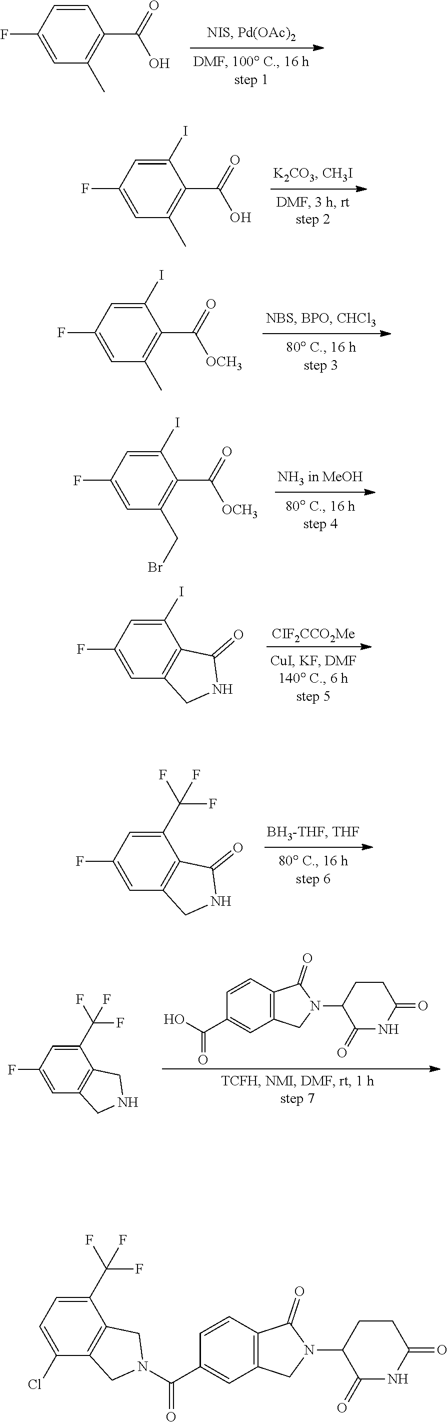

Step 1. Preparation of 4-fluoro-2-iodo-6-methylbenzoic acid. A solution of 4-fluoro-2-methylbenzoic acid (5 g, 32.438 mmol, 1 equiv), NIS (8.03 g, 35.682 mmol, 1.1 equiv) and Pd(OAc)₂ (0.73 g, 3.244 mmol, 0.1 equiv) in dimethylformamide (50 mL) was stirred for overnight at 100° C. under nitrogen atmosphere. The resulting mixture was diluted with water (300 mL) and extracted with EtOAc (3×150 mL). The combined organic layers were washed with Na₂O₃S₂ (aq) (2×200 mL) and brine (2×50 mL), dried over anhydrous Na₂SO₄. After filtration, the filtrate was concentrated under reduced pressure to afford 4-fluoro-2-iodo-6-methylbenzoic acid (8.5 g, crude) as a yellow solid. LC-MS (ES, m/z)=278.90 [M−1]⁻.

Step 2. Preparation of 4-fluoro-2-iodo-6-methylbenzoate. To a stirred mixture of 4-fluoro-2-iodo-6-methylbenzoic acid (8.5 g, 30.353 mmol, 1 equiv) and K₂CO₃ (12.6 g, 91.169 mmol, 3.00 equiv) in dimethylformamide (85 mL) were added CH₃I (2.83 mL, 45.459 mmol, 1.50 equiv) dropwise at 0° C. under nitrogen atmosphere. The resulting mixture was stirred for 3 h at room temperature under nitrogen atmosphere. The reaction was quenched with Water at 0° C. and extracted with EtOAc (3×150 mL). The combined organic layers were washed with water (2×100 mL), dried over anhydrous Na₂SO₄. After filtration, the filtrate was concentrated under reduced pressure. The residue was purified by silica gel column chromatography, eluted with PE/EA (9:1) to afford methyl 4-fluoro-2-iodo-6-methylbenzoate (4.5 g, 50%) as a brown oil. ¹H NMR (400 MHz, DMSO-d₆) δ 7.64 (ddd, J=8.3, 2.5, 0.7 Hz, 1H), 7.24 (ddd, J=9.6, 2.5, 0.8 Hz, 1H), 3.87 (s, 3H), 2.27 (t, J=0.7 Hz, 3H).

Step 3. Preparation of methyl 2-(bromomethyl)-4-fluoro-6-iodobenzoate. To a stirred mixture of methyl 4-fluoro-2-iodo-6-methylbenzoate (2.8 g, 9.522 mmol, 1 equiv) and BPO (0.24 g, 0.952 mmol, 0.1 equiv) in CHCl₃ (30 mL) was added NBS (2.54 g, 14.283 mmol, 1.5 equiv) in portions at room temperature under air atmosphere. The resulting mixture was stirred overnight at 80° C. under nitrogen atmosphere. The mixture was allowed to cool down to room temperature. The resulting mixture was diluted with water (200 mL) and extracted with DCM (3×100 mL). The combined organic layers were washed with brine (2×200 mL), dried over anhydrous Na₂SO₄. After filtration, the filtrate was concentrated under reduced pressure. The residue was purified by reversed-phase flash chromatography to afford methyl 2-(bromomethyl)-4-fluoro-6-iodobenzoate (2.3 g, 65%) as an off-white solid. ¹H NMR (400 MHz, DMSO-d₆) δ 7.91 (dd, J=8.5, 0.9 Hz, 1H), 7.41 (d, J=8.4 Hz, 1H), 4.58 (s, 2H), 3.95 (s, 3H).

Step 4. Preparation of 5-fluoro-7-iodoisoindolin-1-one. A mixture of methyl 2-(bromomethyl)-4-fluoro-6-iodobenzoate (2.3 g, 6.184 mmol, 1 equiv) and NH₃ (7M) in MeOH (20 mL) was stirred overnight at 80° C. under nitrogen atmosphere. The mixture was allowed to cool down to room temperature. The mixture was concentrated under reduced pressure. The residue was purified by silica gel column chromatography, eluted with PE/EA (1:2) to afford 5-fluoro-7-iodoisoindolin-1-one (1.0 g, 58%) as a yellow solid. LC-MS (ES, m/z)=277.90 [M+1]⁺.

Step 5. Preparation of 5-fluoro-7-(trifluoromethyl)isoindolin-1-one. To a stirred mixture of 5-fluoro-7-iodoisoindolin-1-one (1 g, 3.610 mmol, 1 equiv) and methyl 2-chloro-2,2-difluoroacetate (1.04 g, 7.220 mmol, 2 equiv) in DMF (10 mL) was added CuI (0.82 g, 4.332 mmol, 1.2 equiv) and KF (251.7 mg, 4.332 mmol, 1.20 equiv) at room temperature under nitrogen atmosphere. The resulting mixture was stirred for 6 h at 140° C. under nitrogen atmosphere. The mixture was allowed to cool down to room temperature. The resulting mixture was diluted with water (100 mL) and extracted with EtOAc (3×50 mL). The combined organic layers were concentrated under reduced pressure. The residue was purified by reversed-phase flash chromatography to afford 5-fluoro-7-(trifluoromethyl)isoindolin-1-one (300 mg, 38%) as an grey solid. LC-MS (ES, m/z)=217.80 [M−1]⁻.

Step 6. Preparation of 6-fluoro-4-(trifluoromethyl)isoindoline. To a stirred solution of 5-fluoro-7-(trifluoromethyl)isoindolin-1-one (275 mg, 1.255 mmol, 1 equiv) in THF (3 mL) was added BH₃-THF (6.27 mL, 6.275 mmol, 5 equiv) under nitrogen atmosphere. The resulting mixture was stirred overnight at 80° C. under nitrogen atmosphere. The reaction was quenched with 1N HCl at 0° C. The resulting mixture was concentrated under reduced pressure. The residue was purified by reversed-phase flash chromatography to afford 6-fluoro-4-(trifluoromethyl)isoindoline (60 mg, 23%) as a yellow solid. LC-MS (ES, m/z)=206.0 [M+1]⁺.

Step 7. Preparation of 3-(5-(6-fluoro-4-(trifluoromethyl)isoindoline-2-carbonyl)-1-oxoisoindolin-2-yl)piperidine-2,6-dione. To a stirred mixture of 6-fluoro-4-(trifluoromethyl)isoindoline (53.4 mg, 0.260 mmol, 1.50 equiv) and 2-(2,6-dioxopiperidin-3-yl)-1-oxo-3H-isoindole-5-carboxylic acid (50 mg, 0.173 mmol, 1.00 equiv) in DMF (1 mL) were added NMI (71.2 mg, 0.867 mmol, 5.00 equiv) and TCFH (73.0 mg, 0.260 mmol, 1.50 equiv) in portions at room temperature under nitrogen atmosphere. The resulting mixture was stirred for 1 h at room temperature under nitrogen atmosphere. The resulting mixture was diluted with water (50 mL) and extracted with EtOAc (3×30 mL). The combined organic layers were concentrated under reduced pressure. The crude product was purified by Prep-HPLC to afford 3-(5-(6-fluoro-4-(trifluoromethyl)isoindoline-2-carbonyl)-1-oxoisoindolin-2-yl)piperidine-2,6-dione (8.4 mg, 10%) as an off-white solid. LC-MS (ES, m/z)=474.05 [M−1]⁻. ¹H NMR (400 MHz, DMSO-d₆) δ 11.01 (s, 1H), 7.88-7.81 (m, 2H), 7.79-7.48 (m, 3H), 5.16 (dd, J=13.4, 5.0 Hz, 1H), 4.97 (d, J=10.1 Hz, 2H), 4.85 (d, J=20.5 Hz, 2H), 4.54 (dd, J=17.7, 3.8 Hz, 1H), 4.41 (d, J=17.5 Hz, 1H), 2.93 (ddd, J=17.5, 13.4, 5.3 Hz, 1H), 2.62 (d, J=17.5 Hz, 1H), 2.42 (td, J=13.1, 4.0 Hz, 1H), 2.08-2.00 (m, 1H).

### II. Protein-Degrading Effects of the Compounds

### a-1. AML Cell Viability

### Cell Culture

MV4-11 cells were obtained from ATCC and grown in IMDM supplemented with 10% FBS. The cells were maintained at 37° C. under 5% CO₂.

### Cell Viability Assay

The efficacy of the present disclosure to reduce viability of acute myeloid leukemia (AML) cells was assessed using CellTiter-Glo Cell Viability Assay (Promega) according to the manufacturer's protocol. Briefly, Compounds of the present disclosure or reference compounds was added to 3,000 MV-4-11 cells in a white 96-well plate (8-point dose response, top concentration 31.6 μM, half-log serial dilution, final DMSO concentration 0.1%) along with the CellTiter-Glo reagent (final concentration of 1×). The luminescence was normalized to that of DMSO-treated samples. IC₅₀ values were obtained using three-parameter non-linear regressions using Prism (GraphPad).

The efficacy of additional compounds of the present disclosure in reducing AML cell viability was determined by treating MV-4-11 cells at various concentrations (final DMSO concentration of 0.1%). Cell viability was measured using the CellTiter-Glo reagent after 44 hours of treatment. The results are summarized in TABLE 1 below. The compounds are classified based on IC₅₀ values as follows.
A: IC₅₀ is less than or equal to 1×10⁻⁷;
B: IC₅₀ is greater than 1×10⁻⁷ & less than or equal to 1×10⁻⁶;
C: IC₅₀ is greater than 1×10⁻⁶;
NC: not calculated (no activity detected, <25% viability reduction at 1 uM)

### b-1. GSPT1 Degradation

### Cell Culture

MV-4-11 cells were obtained from ATCC and grown in IMDM supplemented with 10% FBS. The cells were maintained at 37° C. under 5% CO₂.

### Western Blot Assay

Compounds of the present disclosure was added to cells (final DMSO concentration of 0.1%) and incubated for 6 hours. GSPT1 levels were determined in MV-4-11 cells using Western Blot analysis. Briefly, whole cell lysates were prepared in lysis buffer (1×RIPA buffer [Thermo, 89900], 1×Pierce protease inhibitor tablets EDTA-free [Thermo, A32965]). 6-20 μg of the lysates was analyzed using Western Blot with antibodies against GSPT1 (Abcam, ab234433 and ab126090) and GAPDH (CST, #2118). Protein levels were normalized to GAPDH levels.

The efficacy of GSPT1 degradation of a part of compounds of the present disclosure was determined by treating MV-4-11 cells at 1 μM (final DMSO concentration of 0.1%) for 6 hours. GSPT1 levels were determined using Western Blot and normalized to GAPDH levels. The results are summarized in TABLE 1. The compounds are classified based on amount of GSPT1 degradation (% reduction of GSPT1 compared to DMSO) as follows.
A: Degradation is greater than or equal to 75%;
B: Degradation is less than 75% & greater than or equal to 50%;
C: Degradation is less than 50%;
ND: No degradation

TABLE 1

| Example No. of | Cell viability | GSPT1 degradation |
|---|---|---|
| Compound | classification | classification |
| Example 1 | A | C |
| Example 2 | B | C |
| Example 4 | A | C |
| Example 8 | A | A |
| Example 11 | A | A |
| Example 12 | A | A |
| Example 21 | A | A |
| Example 22 | A | A |
| Example 28 | A | B |
| Example 31 | B | B |
| Example 32 | A | A |
| Example 56 | B | B |
| Example 57 | B | B |
| Example 58 | A | A |
| Example 59 | A | A |
| Example 60 | B | B |
| Example 61 | A | A |
| Example 62 | NC | C |
| Example 74 | A | A |
| Example 75 | A | A |
| Example 76 | B | C |
| Example 77 | A | A |
| Example 78 | B | B |
| Example 79 | B | B |
| Example 80 | B | B |
| Example 81 | C | C |
| Example 82 | A | B |
| Example 83 | A | C |
| Example 84 | B | C |
| Example 85 | B | C |

### b-2. GSPT1 Degradation

### Western Blot Assay

Compounds of the present disclosure was added to cells (final DMSO concentration of 0.1%) and incubated for 4 hours. GSPT1 levels were determined in MV-4-11 cells using Western Blot analysis. Briefly, whole cell lysates were prepared in lysis buffer (1×RIPA buffer [Thermo, 89900], 1×Pierce protease inhibitor tablets EDTA-free [Thermo, A32965]). 6-20 μg of the lysates was analyzed using Western Blot with antibodies against GSPT1 (Abcam, ab234433 and ab126090) and GAPDH (CST, #2118). Protein levels were normalized to GAPDH levels.

The efficacy of GSPT1 degradation of a part of compounds of the present disclosure was determined by treating MV-4-11 cells at 0.1 μM (final DMSO concentration of 0.1%) for 4 hours. GSPT1 levels were determined using Western Blot and normalized to GAPDH levels. The results are summarized in TABLE 2. The compounds are classified based on amount of GSPT1 degradation (% reduction of GSPT1 compared to DMSO) as follows.
A: Degradation is greater than or equal to 75%;
B: Degradation is less than 75% & greater than or equal to 50%;
C: Degradation is less than 50%;
ND: No degradation.

TABLE 2

| Example No. of | Cell viability | GSPT1 degradation |
|---|---|---|
| Compound | classification | classification |
| Example 63 | B | C |
| Example 64 | A | C |
| Example 65 | A | C |
| Example 66 | A | C |
| Example 67 | A | B |
| Example 68 | A | B |
| Example 69 | A | B |
| Example 70 | A | C |
| Example 71 | C | C |
| Example 72 | B | B |

### c-1. NSCLC Cell Viability

### Cell Culture

HCC827 cells were obtained from Korean Cell Line Bank (KCLB) and grown in RPMI supplemented with 10% FBS. The cells were maintained at 37° C. under 5% CO₂.

### Cell Viability Assay

The efficacy of the present disclosure to reduce viability of Non-Small Cell Lung Cancer (NSCLC) cells was assessed using CellTiter-Glo Cell Viability Assay (Promega) according to the manufacturer's protocol. Briefly, Compounds of the present disclosure or reference compounds was added to 3,000 HCC827 cells in a white 96-well plate (8-point dose response, top concentration 31.6 μM, half-log serial dilution, final DMSO concentration 0.1%) along with the CellTiter-Glo reagent (final concentration of 1×). The luminescence was normalized to that of DMSO-treated samples. IC₅₀ values were obtained using three-parameter non-linear regressions using Prism (GraphPad).

The efficacy of additional compounds of the present disclosure in reducing HCC827 cell viability was determined by treating HCC827 cells at various concentrations (final DMSO concentration of 0.1%). Cell viability was measured using the CellTiter-Glo reagent after 72 hours of treatment. The results are summarized in TABLE 3 below. The compounds are classified based on IC₅₀ values as follows.
A: IC₅₀ is less than or equal to 1×10⁻⁶;
B: IC₅₀ is greater than 1×10⁻⁶ less than or equal to 3×10⁻⁵;
C: IC₅₀ is greater than 3×10⁻⁵;
NC: not calculated (no activity detected, <25% viability reduction at 1 uM)

### b-3. GSPT1 Degradation

### Cell Culture

HCC827 cells were obtained from Korean Cell Line Bank (KCLB) and grown in RPMI supplemented with 10% FBS. The cells were maintained at 37° C. under 5% CO₂.

### Western Blot Assay

Compounds of the present disclosure was added to cells (final DMSO concentration of 0.1%) and incubated for 16 hours. GSPT1 levels were determined in HCC827 cells using Western Blot analysis. Briefly, whole cell lysates were prepared in lysis buffer (1×RIPA buffer [Thermo, 89900], 1×Pierce protease inhibitor tablets EDTA-free [Thermo, A32965]). 6-20 μg of the lysates was analyzed using Western Blot with antibodies against GSPT1 (Abcam, ab234433 and ab126090) and GAPDH (CST, #2118). Protein levels were normalized to GAPDH levels.

The efficacy of GSPT1 degradation of a part of compounds of the present disclosure was determined by treating HCC827 cells at 1 μM (final DMSO concentration of 0.1%) for 16 hours. GSPT1 levels were determined using Western Blot and normalized to GAPDH levels. The results are summarized in TABLE 3. The compounds are classified based on amount of GSPT1 degradation (% reduction of GSPT1 compared to DMSO) as follows.
A: Degradation is greater than or equal to 75%;
B: Degradation is less than 75% & greater than or equal to 50%;
C: Degradation is less than 50%;
ND: No degradation.

TABLE 3

| Example No. of | Cell viability | GSPT1 degradation |
|---|---|---|
| Compound | classification | classification |
| Example 86 | NC | ND |
| Example 87 | A | A |
| Example 88 | A | A |
| Example 89 | NC | ND |
| Example 90 | NC | ND |

### c-2. NSCLC Cell Viability

### Cell Culture

A549 cells were obtained from Korean Cell Line Bank (KCLB) and grown in RPMI supplemented with 10% FBS. The cells were maintained at 37° C. under 5% CO₂.

### Cell Viability Assay

The efficacy of the present disclosure to reduce viability of Non-Small Cell Lung Cancer (NSCLC) cells was assessed using CellTiter-Glo Cell Viability Assay (Promega) according to the manufacturer's protocol. Briefly, Compounds of the present disclosure or reference compounds was added to 3,000 A549 cells in a white 96-well plate (8-point dose response, top concentration 31.6 μM, half-log serial dilution, final DMSO concentration 0.1%) along with the CellTiter-Glo reagent (final concentration of 1×). The luminescence was normalized to that of DMSO-treated samples. IC₅₀ values were obtained using three-parameter non-linear regressions using Prism (GraphPad).

The efficacy of additional compounds of the present disclosure in reducing A549 cell viability was determined by treating A549 cells at various concentrations (final DMSO concentration of 0.1%). Cell viability was measured using the CellTiter-Glo reagent after 72 hours of treatment. The results are summarized in TABLE 4 below. The compounds are classified based on IC₅₀ values as follows.
A: IC₅₀ is less than or equal to 1×10⁻⁶;
B: IC₅₀ is greater than 1×10⁻⁶ less than or equal to 3×10⁻⁵;
C: IC₅₀ is greater than 3×10⁻⁵;
NC: not calculated (no activity detected, <25% viability reduction at 1 uM)

### b-4. GSPT1 Degradation

### Cell Culture

A549 cells were obtained from Korean Cell Line Bank (KCLB) and grown in RPMI supplemented with 10% FBS. The cells were maintained at 37° C. under 5% CO₂.

### Western Blot Assay

Compounds of the present disclosure was added to cells (final DMSO concentration of 0.1%) and incubated for 16 hours. GSPT1 levels were determined in A549 cells using Western Blot analysis. Briefly, whole cell lysates were prepared in lysis buffer (1×RIPA buffer [Thermo, 89900], 1×Pierce protease inhibitor tablets EDTA-free [Thermo, A32965]). 6-20 μg of the lysates was analyzed using Western Blot with antibodies against GSPT1 (Abcam, ab234433 and ab126090) and GAPDH (CST, #2118). Protein levels were normalized to GAPDH levels.

The efficacy of GSPT1 degradation of a part of compounds of the present disclosure was determined by treating A549 cells at 1 μM (final DMSO concentration of 0.1%) for 16 hours. GSPT1 levels were determined using Western Blot and normalized to GAPDH levels. The results are summarized in TABLE 4. The compounds are classified based on amount of GSPT1 degradation (% reduction of GSPT1 compared to DMSO) as follows.
A: Degradation is greater than or equal to 75%;
B: Degradation is less than 75% & greater than or equal to 50%;
C: Degradation is less than 50%;
ND: No degradation.

TABLE 4

| Example No. of | Cell viability | GSPT1 degradation |
|---|---|---|
| Compound | classification | classification |
| Example 91 | A | A |
| Example 92 | A | A |
| Example 93 | B | C |
| Example 94 | NC | |
| Example 95 | NC | |
| Example 96 | B | B |

### a-2. AML Cell Viability

### Cell Culture

MV4-11 cells were obtained from ATCC and grown in IMDM supplemented with 10% FBS. The cells were maintained at 37° C. under 5% CO₂.

### Cell Viability Assay

The efficacy of compounds of the present disclosure in reducing AML cell viability was determined by treating MV-4-11 cells at various concentrations (final DMS 0 concentration of 0.1%). Cell viability was measured using the CellTiter-Glo reagent after 44 hours of treatment. The results are summarized in TABLE 5 below. The compounds are classified based on IC₅₀ values as follows.
A: IC₅₀ is less than or equal to 1×10⁻⁷;
B: IC₅₀ is greater than 1×10⁻⁷ & less than or equal to 1×10⁻⁶;
C: IC₅₀ is greater than 1×10⁻⁶;
NC: not calculated (no activity detected, <25% viability reduction at 1 uM)
N/A: not applicable

### c-2. NSCLC Cell Viability

### Cell Culture

NCI-H1155 cells were obtained from ATCC and grown in RPMI supplemented with 10% FBS. The cells were maintained at 37° C. under 5% CO₂.

### Cell Viability Assay

The efficacy of the present disclosure to reduce viability of NCI-H1155 cells was assessed using CellTiter-Glo Cell Viability Assay (Promega) according to the manufacturer's protocol. Briefly, Compounds of the present disclosure or reference compounds was added to 1,500 NCI-H1155 cells in a white 96-well plate (8-point dose response, top concentration 3.16 μM, half-log serial dilution, final DMSO concentration 0.1%) along with the CellTiter-Glo reagent (final concentration of 1×). The luminescence was normalized to that of DMSO-treated samples. IC₅₀ values were obtained using three-parameter non-linear regressions using Prism (GraphPad).

The efficacy of additional compounds of the present disclosure in reducing NCI-H1155 cell viability was determined by treating NCI-H1155 cells at various concentrations (final DMSO concentration of 0.1%). Cell viability was measured using the CellTiter-Glo reagent after 72 hours of treatment. The results are summarized in TABLE 5 below. The compounds are classified based on IC₅₀ values as follows.
A: IC₅₀ is less than or equal to 1×10⁻⁸;
B: IC₅₀ is greater than 1×10⁻⁸ & less than or equal to 1×10⁻⁷;
C: IC₅₀ is greater than 1×10⁻⁷ & less than or equal to 1×10⁻⁶;
D: IC₅₀ is greater than 1×10⁻⁶
NC: not calculated (no activity detected, <25% viability reduction at 1 uM)
N/A: not applicable

### b-5. GSPT1 Degradation

### GSPT1 HiBiT ASSAY

GSPT1_HiBIT_HEK293 cells were culture in DMEM medium with 10% FBS under standard tissue culture conditions. Briefly, Compounds of the present disclosure was added to 3,000 cells in a white 96-well plate (9-point dose response, top concentration 3.16 μM, half-log serial dilution, final DMSO concentration 0.5%) and incubated for 16 hours. GSPT1 level was determined in along with the Nano-Glo HiBiT Lytic Detection system [Promega N3050] with final concentration of 1×. The luminescence was normalized to that of DMSO-treated samples. DC50 values were obtained using three-parameter non-linear regressions using Prism (GraphPad). The results are summarized in TABLE 5. The compounds are classified based on DC₅₀ values as follows.
A: DC₅₀ is less than or equal to 1×10⁻⁷
B: DC₅₀ is greater than 1×10⁻⁷ & less than or equal to 3×10⁻⁷
C: DC₅₀ is greater than 3×10⁻⁷ & less than or equal to 1×10⁻⁶
D: DC₅₀ is greater than 1×10⁻⁶;
NC: not calculated (no activity detected, <25% degradation at 1 uM)
N/A: not applicable

TABLE 5

| | Cell viability | Cell viability | GSPT1 |
|---|---|---|---|
| Example No. of | classification | classification | degradation |
| Compound | (MV-4-11) | (NCI-H1155) | classification |
| Example 103 | N/A | A | A |
| Example 105 | N/A | A | N/A |
| Example 107 | N/A | C | C |
| Example 109 | N/A | B | A |
| Example 112 | N/A | A | A |
| Example 118 | NC | N/A | N/A |
| Example 119 | NC | N/A | N/A |
| Example 120 | NC | N/A | N/A |
| Example 125 | B | N/A | N/A |
| Example 126 | NC | N/A | N/A |
| Example 127 | B | N/A | N/A |
| Example 128 | NC | N/A | N/A |
| Example 129 | NC | N/A | N/A |
| Example 130 | N/A | B | A |
| Example 132 | N/A | A | N/A |
| Example 133 | N/A | A | A |
| Example 134 | N/A | B | N/A |
| Example 137 | N/A | C | C |
| Example 139 | N/A | B | N/A |
| Example 140 | N/A | NC | N/A |
| Example 141 | N/A | C | N/A |
| Example 142 | N/A | NC | N/A |
| Example 143 | N/A | C | N/A |
| Example 144 | N/A | B | N/A |
| Example 145 | N/A | B | D |
| Example 146 | N/A | C | N/A |
| Example 147 | N/A | B | A |
| Example 148 | N/A | B | N/A |
| Example 149 | N/A | B | N/A |
| Example 150 | N/A | C | N/A |
| Example 151 | N/A | B | N/A |
| Example 152 | N/A | B | N/A |
| Example 153 | N/A | D | N/A |
| Example 154 | N/A | NC | N/A |
| Example 155 | N/A | A | N/A |
| Example 156 | N/A | B | N/A |
| Example 157 | N/A | C | N/A |
| Example 158 | N/A | B | B |
| Example 159 | N/A | NC | N/A |
| Example 160 | N/A | C | N/A |
| Example 161 | N/A | B | B |
| Example 162 | N/A | NC | N/A |
| Example 163 | N/A | C | N/A |
| Example 164 | N/A | B | B |
| Example 165 | N/A | C | NC |
| Example 166 | N/A | C | N/A |
| Example 167 | N/A | C | N/A |
| Example 168 | N/A | C | N/A |
| Example 169 | N/A | B | D |
| Example 170 | N/A | C | N/A |
| Example 171 | N/A | C | N/A |
| Example 172 | N/A | B | N/A |
| Example 173 | N/A | C | N/A |
| Example 174 | N/A | C | C |
| Example 175 | N/A | C | N/A |
| Example 176 | N/A | C | C |
| Example 177 | N/A | C | N/A |
| Example 178 | N/A | B | C |
| Example 179 | N/A | C | N/A |
| Example 180 | N/A | NC | N/A |
| Example 181 | N/A | NC | N/A |
| Example 182 | N/A | A | A |
| Example 183 | N/A | B | N/A |
| Example 184 | N/A | B | B |
| Example 185 | N/A | C | D |
| Example 186 | N/A | NC | N/A |
| Example 187 | N/A | NC | N/A |
| Example 188 | N/A | NC | N/A |
| Example 189 | N/A | C | D |
| Example 190 | N/A | NC | N/A |
| Example 191 | N/A | C | N/A |
| Example 192 | N/A | C | N/A |
| Example 193 | N/A | C | N/A |
| Example 194 | N/A | C | N/A |
| Example 195 | N/A | B | A |
| Example 196 | N/A | B | A |
| Example 197 | N/A | C | NC |
| Example 198 | N/A | NC | NC |
| Example 199 | N/A | C | NC |
| Example 200 | N/A | C | NC |
| Example 201 | N/A | C | D |
| Example 202 | N/A | B | B |
| Example 203 | N/A | C | D |
| Example 204 | N/A | C | B |
| Example 205 | N/A | B | B |
| Example 206 | N/A | D | NC |
| Example 207 | N/A | B | A |
| Example 208 | N/A | A | A |
| Example 209 | N/A | A | A |
| Example 210 | N/A | NC | NC |
| Example 211 | N/A | C | NC |
| Example 212 | N/A | C | N/A |
| Example 213 | N/A | C | N/A |
| Example 214 | N/A | C | N/A |
| Example 215 | N/A | C | N/A |
| Example 216 | N/A | C | N/A |
| Example 217 | N/A | B | B |
| Example 218 | N/A | A | A |
| Example 219 | N/A | B | B |
| Example 220 | N/A | D | N/A |
| Example 221 | N/A | C | N/A |
| Example 222 | N/A | B | N/A |
| Example 223 | N/A | C | N/A |
| Example 224 | N/A | C | N/A |
| Example 225 | N/A | NC | N/A |
| Example 226 | N/A | B | B |
| Example 227 | N/A | A | A |
| Example 228 | N/A | B | N/A |
| Example 229 | N/A | B | N/A |
| Example 230 | N/A | A | A |
| Example 231 | N/A | A | A |
| Example 232 | N/A | C | N/A |
| Example 233 | N/A | NC | N/A |
| Example 234 | N/A | B | N/A |
| Example 235 | N/A | A | A |
| Example 236 | N/A | A | A |
| Example 237 | N/A | NC | N/A |
| Example 238 | N/A | C | N/A |
| Example 239 | N/A | C | N/A |
| Example 240 | N/A | A | A |
| Example 241 | N/A | B | A |
| Example 242 | N/A | B | N/A |
| Example 243 | N/A | NC | N/A |
| Example 244 | N/A | C | N/A |
| Example 245 | N/A | B | N/A |
| Example 246 | N/A | B | N/A |
| Example 247 | N/A | B | N/A |
| Example 248 | N/A | B | N/A |
| Example 249 | N/A | B | N/A |
| Example 250 | N/A | C | N/A |
| Example 251 | N/A | C | N/A |
| Example 252 | N/A | NC | N/A |
| Example 253 | N/A | NC | N/A |
| Example 254 | N/A | B | B |
| Example 255 | N/A | B | A |
| Example 256 | N/A | C | N/A |
| Example 258 | N/A | C | B |
| Example 259 | N/A | D | N/A |
| Example 260 | N/A | C | N/A |
| Example 261 | N/A | B | B |
| Example 262 | N/A | NC | N/A |
| Example 263 | N/A | B | B |
| Example 264 | N/A | B | A |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A compound of Formula (I): 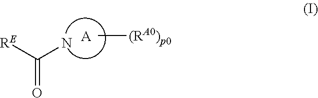
wherein
R^{E} is selected from a group consisting of 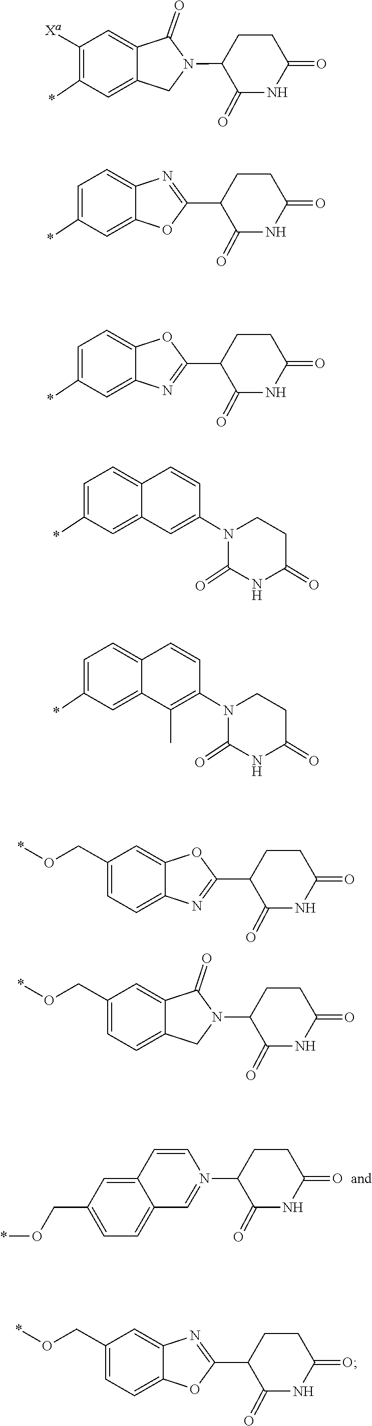
X^{a} is H or F;
Ring A is a monocyclic or bicyclic ring (e.g., fused, spiro, bridged) selected from 4-12 membered saturated, partially unsaturated heterocyclic ring, or heteroaryl ring having 0-4 heteroatoms, in addition to the nitrogen already depicted in Ring A, independently selected from nitrogen, oxygen, and sulfur, wherein 0-4 methylene moieties (—CH₂—) contained in the Ring A may be independently replaced with oxo (—C═O), —NH—, —N—C₁₋₃ alkyl-, or —O—;
R^{A0} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, N(R^{b})SO₂R^{c}, or a monocyclic or bicyclic ring (e.g., fused, spiro, bridged) optionally substituted with one or more R^{d};
wherein the monocyclic or bicyclic ring is selected from a 3-7 membered saturated or partially unsaturated monocyclic carbocyclic ring, a 7-11 membered saturated or partially unsaturated bicyclic carbocyclic ring, a 4-7 membered saturated or partially unsaturated monocyclic heterocyclic ring having 1-4 heteroatoms independently selected from nitrogen, oxygen, and sulfur, a 7-11 membered saturated or partially unsaturated bicyclic heterocyclic ring having 1-5 heteroatoms independently selected from nitrogen, oxygen, and sulfur, a benzene (i.e., phenyl) ring, a 8-10 membered bicyclic aryl ring, a 5-6 membered monocyclic heteroaryl ring having 1-4 heteroatoms independently selected from nitrogen, oxygen, and sulfur, and a 8-10 membered bicyclic heteroaryl ring having 1-5 heteroatoms independently selected from nitrogen, oxygen, and sulfur,
two of R^{A0} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
a subscript p0 is 0, 1, 2, 3, 4, 5, 6, 7, 8, 9, or 10;
each stereocenter in the compound of Formula above is independently the R-enantiomer, the S-enantiomer or a mixture of R- and S-enantiomers; and
each double bond in the compound of Formula above is independently cis or trans,
or a pharmaceutically acceptable salt, enantiomer, stereoisomer or prodrug thereof.

2. The compound of claim 1, wherein the compound comprises additional substituent of an optionally substituted ring of Ring A_{1B} as depicted in the Formula (I-a) below: 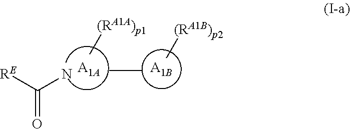
wherein
Ring A_{1A} is a monocyclic or bicyclic ring (e.g., fused, spiro, bridged) selected from 4-12 membered saturated, partially unsaturated heterocyclic ring, or heteroaryl ring having 0-4 heteroatoms, in addition to the nitrogen already depicted in Ring A_{1A}, independently selected from nitrogen, oxygen, and sulfur, wherein 0-4 methylene moieties (—CH₂—) contained in the Ring A_{1A} may be independently replaced with oxo (—C═O), —NH—, —N—C₁₋₃ alkyl-, or —O—;
Ring A_{1B} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur, wherein 0-3 methylene moieties (—CH₂—) contained in the Ring A_{1B} may be replaced with oxo (—C═O), —NH—, —N—C₁₋₃ alkyl-, —O—;
each R^{A1A} and R^{A1B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N=S(O)R^{b}R^{c}, ═NR^{b}, SO₂N(R^{b})R^{c}, or N(R^{b})SO₂R^{c},
two of R^{A1A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
two of R^{A1B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
each subscript p1 and p2 is independently 0, 1, 2, 3, 4, 5, 6, 7, 8, 9, or 10;
or a pharmaceutically acceptable salt, enantiomer, stereoisomer or prodrug thereof.

3. The compound of claim 2, wherein the compound has the structure of Formula (I-a-1): 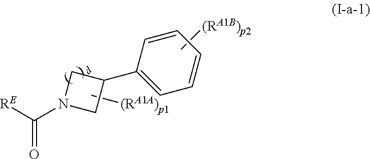
wherein
each R^{A1A} and R^{A1B} is independently hydrogen, deuterium, halogen, C₁-C₆ haloalkyl, or C₁-C₆ haloalkoxy;
a subscript d is 1 or 2;
each subscript p1 and p2 is independently 0, 1, 2, or 3;
or a pharmaceutically acceptable salt, enantiomer, stereoisomer or prodrug thereof.

4. The compound of claim 3, wherein R^{E} is 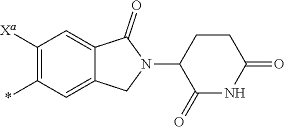
or a pharmaceutically acceptable salt, enantiomer, stereoisomer or prodrug thereof.

5. The compound of claim 1, wherein the compound has the structure of Formula (I-b): 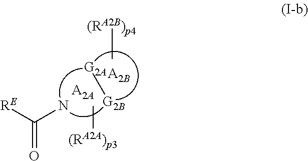
wherein
Ring A_{2A} is a monocyclic heterocyclic ring selected from 3-7 membered saturated, partially unsaturated heterocyclic ring, or 5-6 membered heteroaryl ring having 0-2 heteroatoms, in addition to the nitrogen already depicted in Ring A_{2A}, independently selected from nitrogen, oxygen, and sulfur, wherein 0-2 methylene moieties (—CH₂—) contained in the Ring A_{2A} may be independently replaced with oxo (—C═O), —NH—, —N—C₁₋₃ alkyl-, or —O—;
Ring A_{2B} is a monocyclic ring selected from 3-7 membered saturated or partially unsaturated carbocyclic ring, a 4-7 membered saturated or partially unsaturated monocyclic heterocyclic ring having 1-4 heteroatoms independently selected from nitrogen, oxygen and sulfur, a benzene (i.e., phenyl) ring, or 5-6 membered heteroaryl ring having 1-4 heteroatoms independently selected from nitrogen, oxygen and sulfur, wherein 0-3 methylene moieties (—CH₂—) contained in the Ring A_{2B} may be replaced with oxo (—C═O), —NH—, —N—C₁₋₃ alkyl-, or —O—;
Ring A_{2A} and Ring A_{2B} taken together form a fused-heterocyclic with two shared/border atoms between Ring A_{2A} and Ring A_{2B}, including G_{2A} and G_{2B}, wherein each shared/border atoms can be carbon or heteroatom independently selected from nitrogen, oxygen, and sulfur;
each R^{A2A} and R^{A2B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N=S(O)R^{b}R^{c}, ═NR^{b}, SO₂N(R^{b})R^{c}, or N(R^{b})SO₂R^{c},
two of R^{A2A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
two of R^{A2B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
each subscript p3 and p4 is independently 0, 1, 2, 3, 4, 5, 6, 7, 8, 9, or 10;
or a pharmaceutically acceptable salt, enantiomer, stereoisomer or prodrug thereof.

6. The compound of claim 5, wherein the compound has the structure of selected from Formula (I-b-1) to Formula (I-b-11): 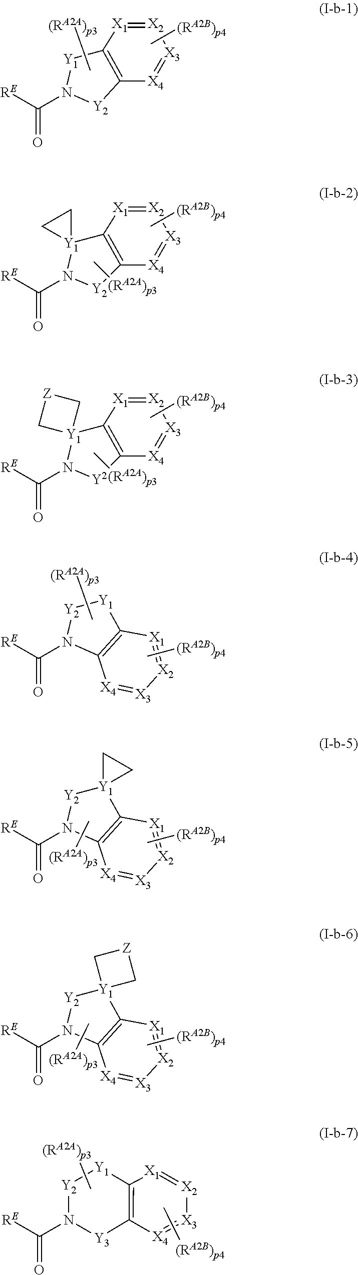 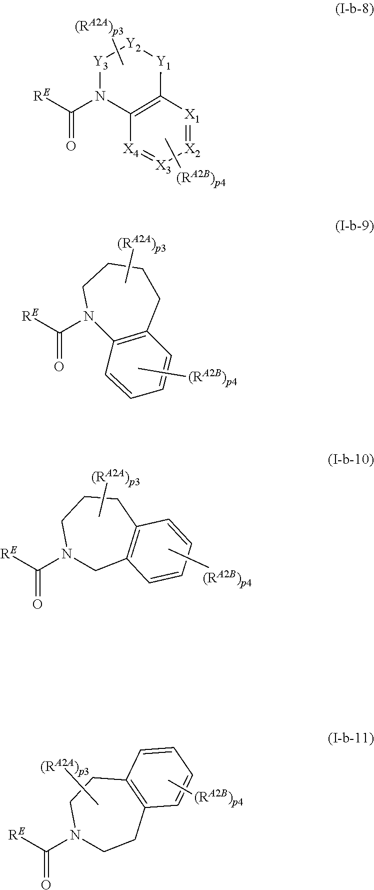
wherein
each of X₁, X₂, X₃ and X₄ is independently N or CH;
each of Y₁, Y₂ and Y₃ is independently O, NH, N—C₁₋₃ alkyl or CH₂;
each of Z is independently O, NH, N—C₁₋₃ alkyl or CH₂;
or a pharmaceutically acceptable salt, enantiomer, stereoisomer or prodrug thereof.

7. The compound of claim 6, wherein each R^{A2A} and R^{A2B} is independently hydrogen, deuterium, halogen, C₁-C₄ alkyl (i.e., straight or branched), C₁-C₃ alkoxy, C₁-C₃ haloalkyl, C₁-C₃ haloalkoxy, cyano, or —(CH₂)₁₋₂—NH—C(O)—(C₁₋₃ alkyl);
or a pharmaceutically acceptable salt, enantiomer, stereoisomer or prodrug thereof.

8. The compound of claim 1, wherein the compound has the structure of Formula (I-c): 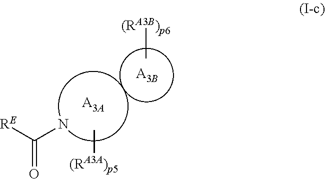
wherein
each Ring A_{3A} and Ring A_{3B} is monocyclic ring selected from 4-7 membered saturated or partially unsaturated ring having 0-4 heteroatoms, in addition to the nitrogen already depicted in Ring A_{3A}, independently selected from nitrogen, oxygen, and sulfur, wherein 0-4 methylene moieties (—CH₂—) contained in the Ring A_{3A} or Ring A_{3B} may be independently replaced with oxo (—C═O), —NH—, —N—C₁₋₃ alkyl-, or —O—;
Ring A_{3A} and Ring A_{3B} taken together form a spiro-heterocyclic with one shared/border carbon atom between Ring A_{3A} and Ring A_{3B};
each R^{A3A} and R^{A3B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N=S(O)R^{b}R^{c}, ═NR^{b}, SO₂N(R^{b})R^{c}, or N(R^{b})SO₂R^{c},
two of R^{A3A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
two of R^{A3B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
each subscript p5 and p6 is independently 0, 1, 2, 3, 4, 5, 6, 7, 8, 9, or 10;
or a pharmaceutically acceptable salt, enantiomer, stereoisomer or prodrug thereof.

9. The compound of claim 8, wherein the compound comprises additional substituent of an optionally substituted ring of Ring A_{3C} as depicted in the Formula (I-c 1) below: 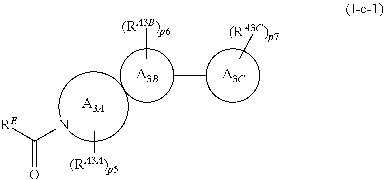
wherein
Ring A_{3C} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur, wherein 0-3 methylene moieties (—CH₂—) contained in the Ring A_{3C} may be independently replaced with oxo (—C═O), —NH—, —N—C₁₋₃ alkyl-, or —O—;
R^{A3C} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c},
two of R^{A3C} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₅-C₁₃ spiroalkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
a subscript p7 is 0, 1, 2, 3, 4, 5, 6, 7, 8, 9, or 10;
or a pharmaceutically acceptable salt, enantiomer, stereoisomer or prodrug thereof.

10. The compound of claim 8, wherein the compound has the structure of Formula (I-c-2) or (I-c-3): 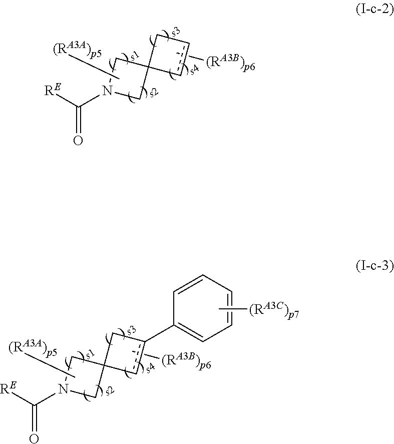
wherein
each subscript s1, s2, s3 and s4 is independently 1 or 2;
a spiro-heterocyclic ring containing the nitrogen already depicted in Formula (I-c-2) or (I-c-3) above, optionally comprises one double bond therein depicted as dotted line, and 0-2 methylene moieties (—CH₂—) contained in the ring may be replaced with oxo (—C═O), —NH—, —N—C₁₋₃ alkyl-, —O—;
or a pharmaceutically acceptable salt, enantiomer, stereoisomer or prodrug thereof.

11. The compound of claim 10, wherein each R^{A3A}, R^{A3B} and R^{A3C} is independently hydrogen, deuterium, halogen, C₁-C₄ alkyl (i.e., straight or branched), C₁-C₃ alkoxy, C₁-C₃ haloalkyl, C₁-C₃ haloalkoxy, or cyano;
or a pharmaceutically acceptable salt, enantiomer, stereoisomer or prodrug thereof.

12. The compound of claim 8, wherein the compound has the structure selected from Formula (I-c-4) to Formula (I-b-14): 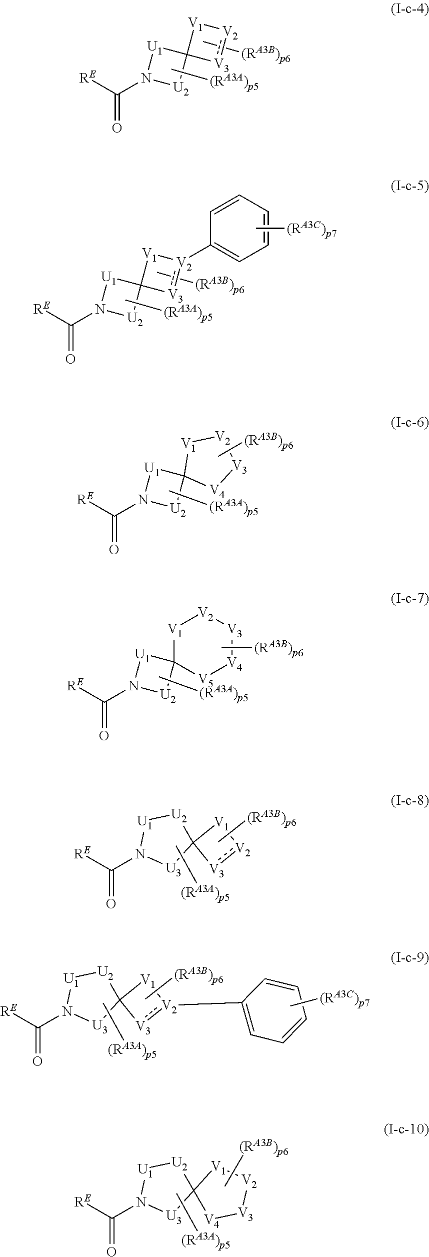 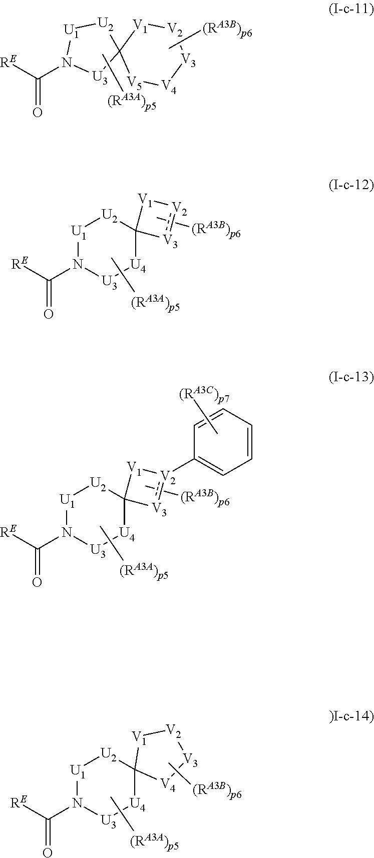
wherein
each of U₁, U₂, U₃ and U₄ is independently C═O, O, NH, N—C₁₋₃ alkyl or CH₂;
each of V₁, V₂, V₃, V₄ and V₅ is independently C═O, O, NH, N—C₁₋₃ alkyl or CH₂;
a spiro-heterocyclic ring containing the nitrogen already depicted in Formula (I-c-4) to (I-c-14) above, optionally comprises one double bond therein depicted as dotted line,
or a pharmaceutically acceptable salt, enantiomer, stereoisomer or prodrug thereof.

13. The compound of claim 1, wherein the compound has the structure of Formula (I-d): 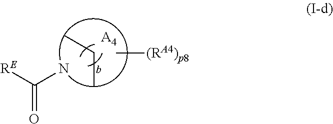
wherein
Ring A₄ is fully saturated 5-10 membered bridged-heterocyclic ring having 0-3 heteroatoms, in addition to the nitrogen already depicted in Ring A₄, independently selected from nitrogen, oxygen, and sulfur, wherein 0-3 methylene moieties (—CH₂—) contained in the Ring A₄ may be independently replaced with oxo (—C═O), —NH—, —N—C₁₋₃ alkyl-, or —O—;
a subscript b is 1, 2 or 3;
R^{A4} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, ORE, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c},
two of R^{A4} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
a subscript p8 is 0, 1, 2, 3, 4, 5, 6, 7, 8, 9, or 10;
or a pharmaceutically acceptable salt, enantiomer, stereoisomer or prodrug thereof.

14. The compound of claim 13, wherein the compound has the structure of Formula (I-d-1) or (I-d-2): 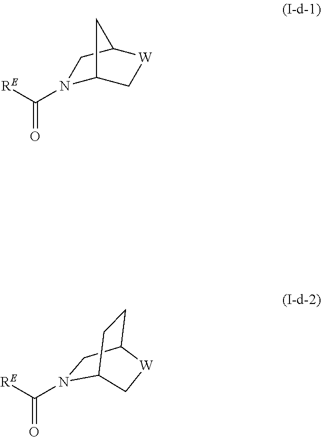
wherein each W is independently O, NH, N—C₁₋₃ alkyl or CH₂;
or a pharmaceutically acceptable salt, enantiomer, stereoisomer or prodrug thereof.

15. A compound of Formula (II): 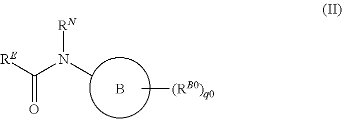
wherein
R^{E} is selected from groups consisting of 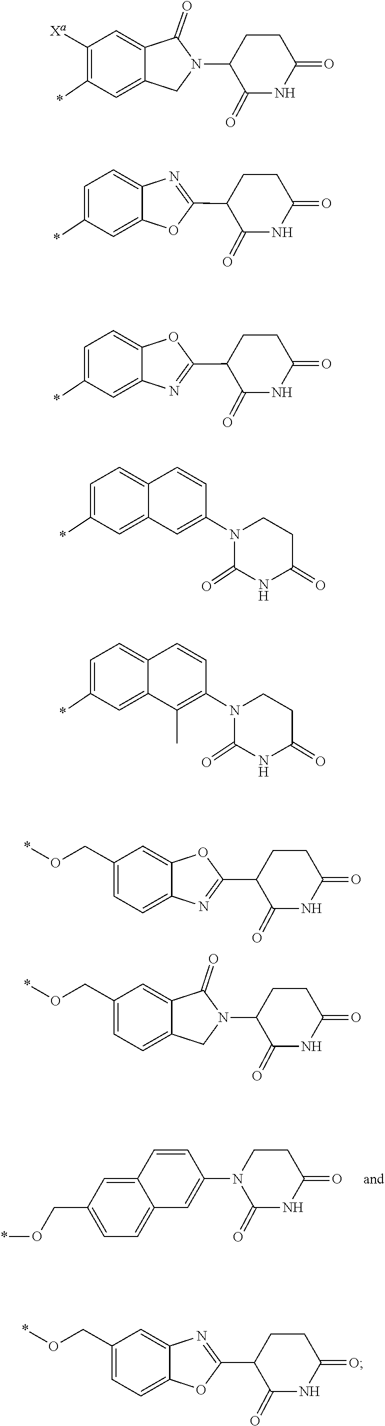
X^{a} is H or F;
R^{N} is hydrogen or C₁-C₃ alkyl;
Ring B is a monocyclic or bicyclic ring (e.g., fused, spiro, bridged) selected from 4-12 membered saturated, partially unsaturated ring having 0-4 heteroatoms independently selected from nitrogen, oxygen, and sulfur, wherein 0-4 methylene moieties (—CH₂—) contained in the Ring B may be replaced with oxo (—C═O), —NH—, —N—C₁₋₃ alkyl-, —O—;
R^{B0} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, N(R^{b})SO₂R^{c}, or a monocyclic or bicyclic ring (e.g., fused, spiro, bridged) optionally substituted with one or more R^{d};
wherein the monocyclic or bicyclic ring is selected from a 3-7 membered saturated or partially unsaturated monocyclic carbocyclic ring, a 7-11 membered saturated or partially unsaturated bicyclic carbocyclic ring, a 4-7 membered saturated or partially unsaturated monocyclic heterocyclic ring having 1-4 heteroatoms independently selected from nitrogen, oxygen, and sulfur, a 7-11 membered saturated or partially unsaturated bicyclic heterocyclic ring having 1-5 heteroatoms independently selected from nitrogen, oxygen, and sulfur, a benzene (i.e., phenyl) ring, a 8-10 membered bicyclic aryl ring, a 5-6 membered monocyclic heteroaryl ring having 1-4 heteroatoms independently selected from nitrogen, oxygen, and sulfur, and a 8-10 membered bicyclic heteroaryl ring having 1-5 heteroatoms independently selected from nitrogen, oxygen, and sulfur,
two of R^{B0} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
a subscript q0 is 0, 1, 2, 3, 4, 5, 6, 7, 8, 9, or 10;
each stereocenter in the compound of Formula above is independently the R-enantiomer, the S-enantiomer or a mixture of R- and S-enantiomers; and
each double bond in the compound of Formula above is independently cis or trans,
or a pharmaceutically acceptable salt, enantiomer, stereoisomer or prodrug thereof.

16. The compound of claim 15, wherein the compound has the structure of Formula (II-b): 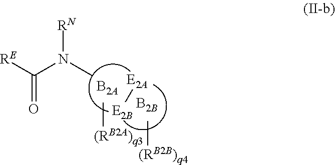
wherein
Ring B_{2A} is a monocyclic heterocyclic ring selected from 3-7 membered saturated, partially unsaturated heterocyclic ring, or 5-6 membered heteroaryl ring having 0-2 heteroatoms independently selected from nitrogen, oxygen, and sulfur, wherein 0-2 methylene moieties (—CH₂—) contained in the Ring B_{2A} may be independently replaced with oxo (—C═O), —NH—, —N—C₁₋₃ alkyl-, or —O—;
Ring B_{2B} is a monocyclic ring selected from 3-7 membered saturated or partially unsaturated carbocyclic ring, a 4-7 membered saturated or partially unsaturated monocyclic heterocyclic ring having 1-4 heteroatoms independently selected from nitrogen, oxygen and sulfur, a benzene (i.e., phenyl) ring, or 5-6 membered heteroaryl ring having 1-4 heteroatoms independently selected from nitrogen, oxygen and sulfur, wherein 0-3 methylene moieties (—CH₂—) contained in the Ring B_{2B} may be replaced with oxo (—C═O), —NH—, —N—C₁₋₃ alkyl-, or —O—;
Ring B_{2A} and Ring B_{2B} taken together form a fused-heterocyclic with two shared/border atoms between Ring B_{2A} and Ring B_{2B}, including E_{2A} and E_{2B}, wherein each shared/border atoms can be carbon or heteroatom independently selected from nitrogen, oxygen, and sulfur;
each R^{B2A} and R^{B2B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N=S(O)R^{b}R^{c}, ═NR^{b}, SO₂N(R^{b})R^{c}, or N(R^{b})SO₂R^{c};
two of R^{B2A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
two of R^{B2B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
each subscript q3 and q4 is independently 0, 1, 2, 3, 4, 5, 6, 7, 8, 9, or 10;
or a pharmaceutically acceptable salt, enantiomer, stereoisomer or prodrug thereof.

17. The compound of claim 15, wherein the compound has the structure of Formula (II-c-1): 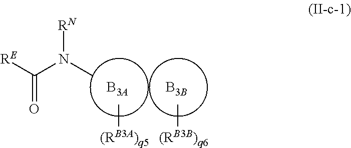
wherein
each Ring B_{3A} and Ring B_{3B} is monocyclic ring selected from 4-7 membered saturated or partially unsaturated ring having 0-4 heteroatoms independently selected from nitrogen, oxygen, and sulfur, wherein 0-4 methylene moieties (—CH₂—) contained in the Ring B_{3A} or Ring B_{3B} may be independently replaced with oxo (—C═O), —NH—, —N—C₁₋₃ alkyl-, or —O—;
Ring B_{3A} and Ring B_{3B} taken together form a spirocyclic or spiro-heterocyclic ring with one shared/border carbon atom between Ring B_{3A} and Ring B_{3B};
each R^{B3A} and R^{B3B} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N=S(O)R^{b}R^{c}, ═NR^{b}, SO₂N(R^{b})R^{c}, or N(R^{b})SO₂R^{c};
two of R^{B3A} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
two of R^{B3B} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
each subscript q5 and q6 is independently 0, 1, 2, 3, 4, 5, 6, 7, 8, 9, or 10;
or a pharmaceutically acceptable salt, enantiomer, stereoisomer or prodrug thereof.

18. The compound of claim 16, wherein the compound comprises additional substituent of an optionally substituted ring of Ring Bic as depicted in the Formula (II-c 2) below: 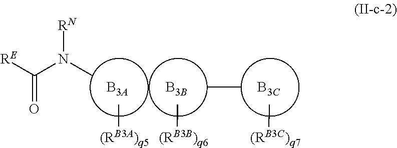
wherein
Ring B_{3C} is a monocyclic ring selected from a 3-7 membered saturated or partially unsaturated carbocyclic ring, a 4-7 membered saturated or partially unsaturated heterocyclic ring having 1-4 heteroatoms independently selected from nitrogen, oxygen, and sulfur, a benzene (i.e., phenyl) ring, a 5-6 membered monocyclic heteroaryl ring having 1-4 heteroatoms independently selected from nitrogen, oxygen, and sulfur, wherein 0-3 methylene moieties (—CH₂—) contained in the Ring B_{3C} may be independently replaced with oxo (—C═O), —NH—, —N—C₁₋₃ alkyl-, or —O—;
R^{B3C} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c},
two of R^{B3C} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₅-C₁₃ spiroalkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
a subscript q7 is 0, 1, 2, 3, 4, 5, 6, 7, 8, 9, or 10;
or a pharmaceutically acceptable salt, enantiomer, stereoisomer or prodrug thereof.

19. The compound of claim 17, wherein the compound has the structure of Formula (II-c-3) or (I-c-4): 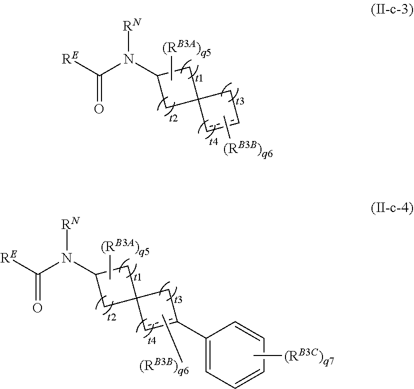
wherein
each subscript t1, t2, t3 and t4 is independently 1 or 2;
spirocyclic or spiro-heterocyclic ring depicted in Formula (II-c-3) or (II-c-4) above, optionally comprises one double bond therein depicted as dotted line, and 0-2 methylene moieties (—CH₂—) contained in the ring may be replaced with oxo (—C═O), —NH—, —N—C₁₋₃ alkyl-, —O—;
or a pharmaceutically acceptable salt, enantiomer, stereoisomer or prodrug thereof.

20. The compound of claim 19, wherein each R^{B3A}, R^{B3B} and R^{B3C} is independently hydrogen, deuterium, halogen, C₁-C₄ alkyl (i.e., straight or branched), C₁-C₃ alkoxy, C₁-C₃ haloalkyl, C₁-C₃ haloalkoxy, or cyano;
or a pharmaceutically acceptable salt, enantiomer, stereoisomer or prodrug thereof.

21. The compound of claim 15, wherein the compound has the structure of Formula (II-d): 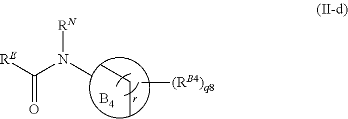
wherein
Ring B₄ is fully saturated 5-10 membered bridged-cyclic ring or bridged-heterocyclic ring having 0-3 heteroatoms independently selected from nitrogen, oxygen, and sulfur, wherein 0-3 methylene moieties (—CH₂—) contained in the Ring B₄ may be independently replaced with oxo (—C═O), —NH—, —N—C₁₋₃ alkyl-, or —O—;
a subscript r is 1, 2 or 3;
R^{B4} is independently hydrogen, deuterium, halogen, C₁-C₁₀ alkyl, C₂-C₆ alkenyl, C₂-C₆ alkynyl, C₁-C₆ haloalkyl, C₁-C₆ alkoxy, C₁-C₆ haloalkoxy, nitro, oxo, cyano, OR^{a}, SR^{a}, alkyl-R^{a}, NH(CH₂)₁₋₁₀R^{a}, C(O)R^{a}, S(O)R^{a}, SO₂R^{a}, C(O)OR^{a}, OC(O)R^{a}, NR^{b}R^{c}, C(O)N(R^{b})R^{c}, N(R^{b})C(O)R^{c}, —P(O)R^{b}R^{c}, -alkyl-P(O)R^{b}R^{c}, -alkyl-O—P(O)(R^{a})(R^{b}), -alkyl-OC(O)N(R^{a})(R^{b}), -alkyl-N(R^{a})C(O)R^{b}, —S(O)(═N(R^{b}))R^{c}, —N═S(O)R^{b}R^{c}, ═NR^{b}, SO₂N(R^{b})R^{c}, or N(R^{b})SO₂R^{c};
two of R^{B4} groups on the same atom or adjacent atoms, taken together with their intervening atoms to which they are attached, may optionally form a spiro or fused cycloalkyl, heterocycloalkyl, heterocycloalkenyl, aryl, or heteroaryl, each optionally substituted with one or more R^{d};
each of R^{a}, R^{b}, R^{c}, and R^{d} independently, is hydrogen, deuterium, C₁-C₆ alkyl, C₂-C₆ alkenyl, C₂-C₆ alkynyl, halogen, amine, nitro, oxo, cyano, hydroxy, ═O, C₁-C₆ alkoxy, C₁-C₆ haloalkoxy, C₁-C₆ haloalkyl, C₁-C₆ aminoalkyl, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; wherein each R is independently hydrogen or C₁-C₆ alkyl;
a subscript q8 is 0, 1, 2, 3, 4, 5, 6, 7, 8, 9, or 10;
or a pharmaceutically acceptable salt, enantiomer, stereoisomer or prodrug thereof.

22. The compound of claim 21, wherein the compound has the structure of Formula (II-d-1) or (II-d-2): 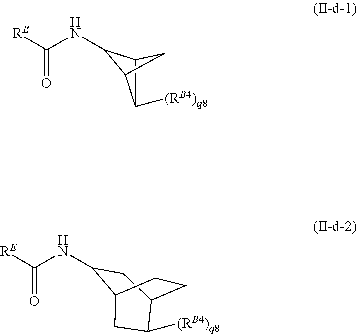
wherein each R^{B4} is independently hydrogen, deuterium, halogen;
pharmaceutically acceptable salt, enantiomer, stereoisomer or prodrug thereof.

23. The compound of claim 1, wherein the compound is selected from [Table A-1], [Table A-2], [Table A-3], [Table A-4], [Table A-5], [Table B-1], [Table B-2], [Table B-3] or [Table B-4] in the specification, pharmaceutically acceptable salt, enantiomer, stereoisomer or prodrug thereof.

24. (canceled)

25. (canceled)

26. (canceled)

27. (canceled)

28. A pharmaceutical composition comprising:
the compound of claim 1, or the pharmaceutically acceptable salt, enantiomer, stereoisomer, prodrug or derivatives thereof, and
optionally a pharmaceutically acceptable excipient or carrier.

29. A method of degrading GSPT1 in a subject in need thereof comprising administering to the subject an effective amount of the compound of claim 1 or the pharmaceutically acceptable salt, enantiomer, stereoisomer, prodrug or derivatives thereof.

30. A method for reducing GSPT1 protein level in a cell comprising contacting the cell with an effective amount of the compound of claim 1 or the pharmaceutically acceptable salt, enantiomer, stereoisomer, prodrug or derivatives thereof.

31. A method of inhibiting GSPT1 activity in a subject in need thereof comprising administering to the subject an effective amount of the compound of claim 1 or the pharmaceutically acceptable salt, enantiomer, stereoisomer, prodrug or derivatives thereof.

32. A method of treating GSPT1-related disease in a subject in need thereof comprising administering to the subject an effective amount of the compound of claim 1 or the pharmaceutically acceptable salt, enantiomer, stereoisomer, prodrug or derivatives thereof.

33. The method of claim 31, wherein the GSPT1-related disease is cancer, proliferative disorder, inflammation, autoimmune disease, viral infections, or immunological disease.

34. The method of claim 32, wherein the GSPT1-related disease is cancer.

35. The method of claim 33, wherein the cancer is selected from the group consisting of gastric adenocarcinoma, colorectal cancer, li-fraumeni syndrome, esophageal cancer, hepatocellular carcinoma, adenocarcinoma, small intestine cancer, pancreatic cancer, lung cancer, brain cancer, ovarian cancer, cervical cancer, prostate cancer, colon cancer, head and neck cancer, stomach cancer, hepatobiliary cancer, rectal cancer, kidney cancer, bladder cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, liver cancer, skin cancer, urothelial carcinoma, renal cancer, renal cell carcinoma, oropharyngeal cancer, carcinoma, melanoma and breast cancer.

36. The method of claim 33, wherein the cancer is selected from the group consisting of breast cancer, colorectal cancer, lung cancer, e.g. SCLC, NSCLC, liver cancer, neuroendocrine cancer, e.g., neuroendocrine prostate cancer (for example, NEPC (castration-resistant neuroendocrine prostate cancer)) and lung neuroendocrine tumors (Lu-NETs), stomach cancer, pancreatic cancer, gastric cancer, esophageal cancer, bladder cancer, skin cancer, and head and neck cancer.
